(19) United States
(12) Patent Application Publication
(10) Pub. No.: US 2012/0231512 A1
(43) Pub. Date: Sep. 13, 2012

(54) PREPARATION OF ALPHA-KETOPIMELIC ACID

(75) Inventors: Petronella Catharina Raemakers-Franken, KJ Budel (NL); Axel Christoph Trefzer, KR Leidschendam (NL); Linda Vermote, HB Sittard (NL)

(73) Assignee: DSM IP ASSETS B.V., Heerlen (NL)

(21) Appl. No.: 13/394,235

(22) PCT Filed: Sep. 10, 2010
(86) PCT No.: PCT/NL10/50573

§ 371 Date: May 8, 2012

(30) Foreign Application Priority Data
Sep. 11, 2009 (EP) 09170078.1

(51) Int. Cl. C12P 7/50; C12P 7/44; C12N 1/21; C12P 17/10; C12N 1/19; C12N 1/15; C12N 15/53; C12P 13/00; C12N 1/13

(52) U.S. Cl. 435/121; 435/143; 435/128; 435/142; 435/252.3; 435/252.33; 435/252.31; 435/252.32; 435/257.2; 435/254.21; 435/254.22; 435/254.23; 435/254.2; 435/254.3; 435/254.5; 435/254.11; 536/23.2

(57) ABSTRACT

The present invention relates to a method for preparing alpha-ketopimelic acid, comprising converting 2-hydroxyheptanedioic acid into alpha-ketopimelic acid, which conversion is catalysed using a biocatalyst. Further, the invention relates to a heterologous cell, comprising a nucleic acid sequence encoding an enzyme having catalytic activity in the conversion of 2-hydroxyheptanedioic acid into alpha-ketopimelic acid. Further, the invention relates to the use of a heterologous cell according to the invention in the preparation of caprolactam, diaminohexane or adipic acid.

The invention relates to a method for preparing alpha-ketopimelic acid (hereinafter also referred to as ‘AKP’; AKP is also known as 2-oxo-heptanedioic acid). The invention further relates to a method for preparing 6-aminocaproic acid (hereinafter also referred to as ‘6-ACA’). The invention also relates to a method for preparation of adipic acid, to a method for preparing 5-formylpentanoic acid (hereinafter also referred to as ‘5-FVA’), to a method for preparing alpha amino-pimelic acid (AAP), and to a method for preparation of diaminohexane (also known as 1,6-hexanediamine). The invention further relates to a heterologous cell which may be used in a method according to the invention. The invention further relates to the use of a heterologous cell in the preparation of ε-caprolactam (hereafter referred to as ‘caprolactam’), adipic acid, or diaminohexane.

Adipic acid (hexanedioic acid) is inter alia used for the production of polyamide. Further, esters of adipic acid may be used in plasticisers, lubricants, solvent and in a variety of polyurethane resins. Other uses of adipic acid are as food acidulants, applications in adhesives, insecticides, tanning and dyeing. Known preparation methods include the oxidation of cyclohexanol or cyclohexanone or a mixture thereof (KA oil) with nitric acid.

Diaminohexane is inter alia used for the production of polyamides such as nylon 6,6. Other uses include uses as starting material for other building blocks (e.g. hexamethylene diisocyanate) and as crosslinking agent for epoxides. A known preparation method proceeds from acrylonitrile via adiponitrile.

Caprolactam is a lactam which may be used for the production of polyamide, for instance nylon-6 or nylon-6,12 (a copolymer of caprolactam and laurolactam). Various manners of preparing caprolactam from bulk chemicals are known in the art and include the preparation of caprolactam from cyclohexanone, toluene, phenol, cyclohexanol, benzene or cyclohexane. These intermediate compounds are generally obtained from mineral oil.

In view of a growing desire to prepare materials using more sustainable technology it would be desirable to provide a method wherein caprolactam, adipic acid or diaminohexane is prepared from an intermediate compound that can be obtained from a biologically renewable source or at least from an intermediate compound that is converted into caprolactam using a biochemical method. Further, it would be desirable to provide a method that requires less energy than conventional chemical processes making use of bulk chemicals from petrochemical origin.

It is known to prepare caprolactam from 6-ACA, e.g. as described in U.S. Pat. No. 6,194,572. As disclosed in WO 2005/068643, 6-ACA may be prepared biochemically by converting 6-aminohex-2-enoic acid (6-AHEA) in the presence of an enzyme having α,β-enoate reductase activity. The 6-AHEA may be prepared from lysine, e.g. biochemically or by pure chemical synthesis. Although the preparation of G-ACA via the reduction of 6-AHEA is feasible by the methods disclosed in WO 2005/068643, the inventors have found that—under the reduction reaction conditions—6-AHEA may spontaneously and substantially irreversibly cyclise to form an undesired side-product, notably β-homoproline. This cyclisation may be a bottleneck in the production of 6-ACA, and may lead to a considerable loss in yield.

The inventors have realised that it is possible to prepare 6-ACA from AKP. AKP can be prepared chemically, e.g. based on a method as described by H. Jäger et al. Chem. Ber. 1959, 92, 2492-2499. AKP can be prepared by alkylating cyclopentanone with diethyl oxalate using sodium ethoxide as a base, refluxing the resultant product in a strong acid (2 M HCl) and recovering the product, e.g. by crystallisation from toluene. However, as indicated above, there is a growing desire to prepare materials using more sustainable technology. Thus, the inventors realised it would be desirable to provide a method wherein AKP is prepared from an intermediate compound that can be obtained from a biologically renewable source.

It is an object of the invention to provide a novel method for preparing AKP, which may be used, in particular, for the preparation of 6-ACA, adipic acid, diaminohexane or another compound.

It is further an object to provide a novel biocatalyst, suitable for catalysing one or more reaction step in a method for preparing AKP.

One or more further objects which may be solved in accordance with the invention will follow from the description below.

The inventors have realised it is possible to prepare AKP using a specific biocatalyst.

Accordingly, the present invention relates a method for preparing alpha-ketopimelic acid (AKP), comprising converting 2-hydroxyheptanedioic acid into alpha-ketopimelic acid (AKP), which conversion is catalysed using a biocatalyst, in particular a heterologous biocatalyst.

AKP prepared in a method of the invention may further be used in the preparation of another compound, or be used as such, e.g. as a chemical for biochemical research or as a pH-buffer compound, e.g. for use in an preparative or analytical separation technique such as liquid chromatography or capillary electrophoresis. In particular, if desired, AKP may be used for the preparation of 5-FVA, AAP (2-aminoheptanedioic acid, also known as alpha-aminopimelic acid), 6-ACA, or adipic acid. Suitable biocatalysts for a biocatalytic preparation of FVA, AAP or G-ACA are for instance found in WO 2009/113855.

Accordingly, the invention further relates to a method for preparing 5-FVA comprising biocatalytically decarboxylating AKP prepared in a method according to the invention thereby forming 5-FVA.

The 5-FVA is for instance a suitable intermediate compound for preparing 6-ACA, caprolactam, diaminohexane or adipic acid.

The AKP may for instance be used as an intermediate in the preparation of AAP.

Accordingly, the invention further relates to a method for preparing AAP comprising biocatalytically transaminating AKP prepared in a method according to the invention, thereby forming AAP.

The AAP is for instance a suitable intermediate compound for preparing 6-ACA, di-amino hexane or caprolactam.

6-ACA may for instance be converted into caprolactam or into diaminohexane.

The invention further relates to a heterologous cell, comprising a nucleic acid sequence encoding an enzyme having catalytic activity in the conversion of 2-hydroxyheptanedioic acid into alpha-ketopimelic acid. This nucleic acid sequence and the encoded enzyme are in general heterologous to the cell.

A cell according to the invention may in particular be used as a biocatalyst in a method for preparing at least one compound selected from the group of AKP, 5-FVA, 6-ACA, AAP, adipic acid, diaminohexane and caprolactam.

In accordance with the invention, no problems have been noticed with respect to an undesired cyclisation of an intermediate product, when forming 6-ACA and optionally caprolactam, resulting in a loss of yield.

It is envisaged that a method of the invention allows a comparable or even better yield than the method described in WO 2005/68643. It is envisaged that a method of the invention may in particular be favourable if a use is made of a living organism—in particular in a method wherein growth and maintenance of the organism is taken into account.

It is further envisaged that in an embodiment of the invention the productivity of 6-ACA (g/l·h formed) in a method of the invention may be improved.

The term “or” as used herein is defined as “and/or” unless specified otherwise.

The term “a” or “an” as used herein is defined as “at least one” unless specified otherwise.

When referring to a noun (e.g. a compound, an additive, etc.) in the singular, the plural is meant to be included. Thus, when referring to a specific moiety, e.g. “compound”, this means “at least one” of that moiety, e.g. “at least one compound”, unless specified otherwise.

When referred herein to carboxylic acids or carboxylates, e.g. 6-ACA, another amino acid, 5-FVA, adipic acid/adipate, succinic acid/succinate, acetic acid/acetate, these terms are meant to include the protonated carboxylic acid (free acid), the corresponding carboxylate (its conjugated base) as well as a salt thereof, unless specified otherwise. When referring herein to amino acids, e.g. 6-ACA, this term is meant to include amino acids in their zwitterionic form (in which the amino group is in the protonated and the carboxylate group is in the deprotonated form), the amino acid in which the amino group is protonated and the carboxylic group is in its neutral form, and the amino acid in which the amino group is in its neutral form and the carboxylate group is in the deprotonated form, as well as salts thereof.

When referring to a compound of which several isomers exist (e.g. a cis and a trans isomer, an R and an S enantiomer), the compound in principle includes all enantiomers, diastereomers and cis/trans isomers of that compound that may be used in the particular method of the invention.

When an enzyme is mentioned with reference to an enzyme class (EC) between brackets, the enzyme class is a class wherein the enzyme is classified or may be classified, on the basis of the Enzyme Nomenclature provided by the Nomenclature Committee of the International Union of Biochemistry and Molecular Biology (NC-IUBMB), which nomenclature may be found at http://www.chem.qmul.ac.uk/iubmb/enzmme/. Other suitable enzymes that have not (yet) been classified in a specified class but may be classified as such, are meant to be included.

If referred herein to a protein or gene by reference to a accession number, this number in particular is used to refer to a protein or gene having a sequence as found in Uniprot on 11 Sep. 2009, unless specified otherwise.

The term “homologue” is used herein in particular for polynucleotides or polypeptides having a sequence identity of at least 30%, preferably at least 40%, more preferably at least 60%, more preferably at least 65%, more preferably at least 70%, more preferably at least 75%, more preferably at least 80%, in particular at least 85%, more in particular at least 90%, at least 91%, at least 92%, at least 93%, at least 94%, at least 95%, at least 96%, at least 97%, at least 98% or at least 99%.

Further, homologues usually have a significant sequence similarity, usually of more than 30%, in particular a sequence similarity of at least 35%, preferably at least 40%, more preferably at least 60%, more preferably at least 65%, more preferably at least 70%, more preferably at least 75%, more preferably at least 80%, in particular at least 85%, more in particular at least 90%, at least 91%, at least 92%, at least 93%, at least 94%, at least 95%, at least 96%, at least 97%, at least 98% or at least 99%.

Homologues generally have an intended function in common with the polynucleotide respectively polypeptide of which it is a homologue, such as encoding the same peptide respectively being capable of catalysing the same reaction (typically the conversion of the same substrate into the same compound) or a similar reaction. A ‘similar reaction’ typically is a reaction of the same type, e.g. a decarboxylation or an aminotransfer. Accordingly, as a rule of thumb, homologous enzymes can be classified in an EC class sharing the first three numerals of the EC class (x.y.z), for example EC 4.1.1 for carboxylyases. Typically, in the similar reaction, a substrate of the same class (e.g. an amine, a carboxylic acid, an amino acid) as the substrate for the reaction to which the similar reaction is similar is converted into a product of the same class as the product of the reaction to which the similar reaction is similar. Similar reactions in particular include reactions that are defined by the same chemical conversion as defined by the same KEGG RDM patterns, wherein the R-atoms and D-atoms describe the chemical conversion (KEGG RDM patterns: Oh, M. et al. (2007) Systematic analysis of enzyme-catalyzed reaction patterns and prediction of microbial biodegradation pathways. J. Chem. Inf. Model., 47, 1702-1712).

The term homologue is also meant to include nucleic acid sequences (polynucleotide sequences) which differ from another nucleic acid sequence due to the degeneracy or experimental adaptation of the genetic code and encode the same polypeptide sequence.

The term “functional analogue” is used herein for nucleic acid sequences that differ from a given sequence of which said analogue is an analogue, yet that encode a peptide (protein, enzyme) having the same amino acid sequence or that encode a homologue of such peptide. In particular, preferred functional analogues are nucleotide sequences having a similar, the same or a better level of expression in a host cell of interest as the nucleotide sequence of which it is referred to as being a functional analogue of. In this respect it is observed that, as the skilled person understands, a better level of expression usually is a higher level of expression if the expression of the peptide (protein, enzyme) is desired. However, in specific embodiment a better level of expression may be a lower expression level since this might be desirable in context of a metabolic pathway in said host cell. The functional analogue can be a naturally occurring sequence, i.e. a wild-type functional analogue, or a genetically modified sequence, i.e. a non-wild type functional analogue. Codon optimised sequences encoding a specific peptide, are generally non-wild type functional analogues of a wild-type sequence, designed to achieve a desired expression level.

Sequence identity or similarity is herein defined as a relationship between two or more polypeptide sequences or two or more nucleic acid sequences, as determined by comparing the sequences. Usually, sequence identities or similarities are compared over the whole length of the sequences, but may however also be compared only for a part of the sequences aligning with each other. In the art, “identity” or “similarity” also means the degree of sequence relatedness between polypeptide sequences or nucleic acid sequences, as the case may be, as determined by the match between such sequences. Preferred methods to determine identity or similarity are designed to give the largest match between the sequences tested. In context of this invention a preferred computer program method to determine identity and similarity between two sequences includes BLASTP and BLASTN (Altschul, S. F. et al., J. Mol. Biol. 1990, 215, 403-410, publicly available from NCBI and other sources (BLAST Manual, Altschul, S., et al., NCBI NLM NIH Bethesda, Md. 20894). Preferred parameters for polypeptide sequence comparison using BLASTP are gap open 10.0, gap extend 0.5, Blosum 62 matrix. Preferred parameters for nucleic acid sequence comparison using BLASTN are gap open 10.0, gap extend 0.5, DNA full matrix (DNA identity matrix).

A heterologous biocatalyst, in particular a heterologous cell, as used herein, is a biocatalyst comprising a heterologous protein or a heterologous nucleic acid (usually as part of the cell's DNA or RNA) The term “heterologous” when used with respect to a nucleic acid sequence (DNA or RNA), or a protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. It is understood that heterologous DNA in a heterologous organism is part of the genome of that heterologous organism. Heterologous nucleic acids or proteins are not endogenous to the cell into which they are introduced, but have been obtained from another cell or synthetically or recombinantly produced. Generally, though not necessarily, such nucleic acids encode proteins that are not normally produced by the cell in which the DNA is transcribed or expressed. Similarly heterologous RNA encodes for proteins not normally expressed in the cell in which the heterologous RNA is present. Heterologous nucleic acids and proteins may also be referred to as foreign nucleic acids or proteins. Any nucleic acid or protein that one of skill in the art would recognise as heterologous or foreign to the cell in which it is expressed is herein encompassed by the term heterologous nucleic acid or protein.

When referred to a an enzyme or another biocatalytic moiety, from a particular source, recombinant enzymes or other recombinant biocatalytic moieties, originating from a first organism, but actually produced in a (genetically modified) second organism, are specifically meant to be included as enzymes or other biocatalytic moieties, from that first organism.

In a method of the invention, a biocatalyst is used, i.e. at least one reaction step in the method is catalysed by a biological material or moiety derived from a biological source, for instance an organism or a biomolecule derived there from. The biocatalyst may in particular comprise one or more enzymes. A biocatalytic reaction may comprise one or more chemical conversions of which at least one is catalyzed by a biocatalyst. Thus the ‘biocatalyst’ may accelerate a chemical reaction in at least one reaction step in the preparation of AKP, at least one reaction step in the preparation of 5-FVA or AAP from AKP, at least one reaction step in the preparation of 6-ACA or adipic acid from 5-FVA, at least one reaction step in the preparation of 6-ACA from AAP, at least one reaction step in the preparation of diaminohexane, or at least one reaction step in the preparation of caprolactam from 6-ACA.

The biocatalyst may be used in any form. In an embodiment, one or more enzymes form part of a living organism (such as living whole cells). The enzymes may perform a catalytic function inside the cell. It is also possible that the enzyme may be secreted into a medium, wherein the cells are present. In an embodiment, one or more enzymes are used isolated from the natural environment (isolated from the organism it has been produced in), for instance as a solution, an emulsion, a dispersion, (a suspension of) freeze-dried cells, a lysate, or immobilised on a support. The use of an enzyme isolated from the organism it originates from may in particular be useful in view of an increased flexibility in adjusting the reaction conditions such that the reaction equilibrium is shifted to the desired side.

Living cells may be growing cells, resting or dormant cells (e.g. spores) or cells in a stationary phase. It is also possible to use an enzyme forming part of a permeabilised cell (i.e. made permeable to a substrate for the enzyme or a precursor for a substrate for the enzyme or enzymes).

The biocatalyst (used in a method of the invention) may in principle be any organism, or be obtained or derived from any organism. This organism may be a naturally occurring organism or a heterologous organism. The heterologous organism is typically a host cell which comprises at least one nucleic acid sequence encoding a heterologous enzyme, capable of catalysing at least one reaction step in a method of the invention. The organism from which the heterologous nucleic acid sequence originates may be may be eukaryotic or prokaryotic. In particular said organisms may be independently selected from animals (including humans), plants, bacteria, archaea, yeasts and fungi.

The host cell may be eukaryotic or prokaryotic. In an embodiment, the host cell is selected from the group of fungi, yeasts, euglenoids, archaea and bacteria. The host cell may in particular be selected from the group of genera consisting of Aspergillus, Penicillium, Ustilago, Cephalosporium, Trichophytum, Paecilomyces, Pichia, Hansenula, Saccharomyces, Candida, Kluyveromyces, Yarrowia, Bacillus, Corynebacterium, Escherichia, Azotobacter, Frankia, Rhizobium, Bradyrhizobium, Anabaena, Synechocystis, Microcystis, Klebsiella, Rhodobacter, Pseudomonas, Thermus, Deinococcus and Gluconobacter.

In particular, the host strain and, thus, host cell for use in a method of the invention may be selected from the group of Escherichia coli, Azotobacter vinelandii, Klebsiella pneumoniae, Anabaena sp., Synechocystis sp., Microcystis aeruginosa, Deinococcus radiourans, Deinococcus geothermalis, Thermus thermophilus, Bacillus sphaericus, Bacillus subtilis, Bacillus amyloliquefaciens, Bacillus methanolicus, Corynebacterium glutamicum, Aspergillus niger, Penicillium chrysogenum, Penicillium notatum, Paecilomyces carneus, Cephalosporium acremonium, Ustilago maydis, Pichia pastoris, Saccharomyces cerevisiae, Kluyveromyces lactis, Candida crucei, Candida maltosa, Yarrowia lipolytica, and Hansenula polymorpha host cells. In particular in an embodiment wherein AKP is to be converted into a further product, for instance 5-FVA, AAP, adipate, diaminohexane or 6-ACA, it is considered advantageous that the host cell is an organism naturally capable of converting AKP to such product or at least capable of catalysing one of the necessary reactions. For instance, Escherichia coli has aminotransferase activity, whereby E. coli may catalyse the formation of AAP from AKP (see also below) or the conversion of 5-FVA (which may be formed in the cell if the cell also contains a suitable decarboxylase, see also below) to 6-ACA. Further, E. coli may have AKP decarboxylase activity (suitable to convert AKP into 5-FVA) and/or aldehydedehydrogenase activity (catalysing the preparation of adipate from 5-FVA).

Further it is considered advantageous that the host cell comprises an enzyme system for synthesising pimelate (a pimelate synthesis pathway) or a part thereof. Pimelate is known as intermediate in biotin biosynthesis and as such, the inventors consider that organisms capable of de-novo synthesis of biotin are expected to also contain a synthetic pathway for pimelate. Pimelate has been described to be produced from fatty acids (via oxidation thereof). This results in a break of the carbon chain and yields the second carboxylic acid functionality (W. R. Streit, P. Entcheva. Biotin in microbes, the genes involved in its biosynthesis, its biochemical role and perspectives for biotechnological production. Appl Microbiol Biotechnol (2003) 61:21-31; Max J. Cryle, Ilme Schlichting. Structural insights from a P450 Carrier Protein complex reveal how specificity is achieved in the P450Biol ACP complex. PNAS (2008) 105 (41): 15696-15701).

Further organisms providing the enzyme system for pimelate synthesis may be selected from genera of the Bacillus sensu lato group, Geobacillus, Brevibacillus and the like (see Table 1 in Zeigler and Perkins, 2008, “Practical Handbook of Microbiology”, Second Edition (E. Goldman and L. Green, eds.), pp 301-329, CRC Press, Boca Raton, Fla.). In particular from Bacillus species represented by the Bacillus sensu stricto group, in particular Bacillus subtilis, Bacillus lentimorbus, Bacillus lentus, Bacillus anthracis, Bacillus firmus, Bacillus pantothenticus, Bacillus cereus, Bacillus circulans, Bacillus coagulans, Bacillus megaterium, Bacillus thuringiensis, Bacillus licheniformis, Bacillus amyloliquefaciens, Bacillus pumilus, Bacillus halodurans (Zeigler and Perkins, 2008, Ibid). More in particular, from Bacillus subtilis 168 and its strain derivatives. Further, organisms providing the enzyme system for pimelate synthesis may also be selected from genera of e.g. Corynebacterium, Lactobacillus, Lactococci, Streptomyces, and Pseudomonas. In particular, a host cell comprising an enzyme system for synthesising pimelate may be selected from the group of gram-positive bacteria (Streit and Entcheva, Appl Microbiol Biotechnol (2003) 61:21-31) For instance, Bacillus sphaericus has been reported to comprise an enzyme system for synthesising pimelate (Gloeckler et al., Gene 87:63-70, 1990). Further, Bacillus subtilis is an example of an organism comprising enzymes for a pimelate synthesis pathway (see e.g. EP-A 635 572).

Gram negative bacteria may also provide pimelic acid. These microbes usually also comprise an enzyme system to prepare pimeloyl-CoA, see for instance for Escherichia coli Otsuka et al., J. Biol. Chem. 263:19577-19585 (1988); O'Regan et al., Nucleic Acids Res. 17:8004 (1989))). Even in case wild-type strains of these bacteria are not capable of producing pimelic acid, by their capacity to prepare pimeloyl-CoA, they may provide a source for pimelate, in that upon hydrolysis of pimeloyl-CoA, pimelate is formed.

In a specific embodiment, a host cell according to the invention comprising an enzyme system for synthesising pimelate is capable of producing one or more lipids which can serve as precursor for pimelate in high yield. The host cell may be naturally capable of said lipid production or have been genetically modified by incorporating one or more genes involved in said lipid production from an organism of which the wild-type is naturally capable of said lipid production. Examples of such organisms include oleaginous yeasts, micro algae, fungi and bacteria.

Suitable micro algae may be selected from the group of Dunalliela bardawil, Chlamydomonas reinhardtii, Prymnesium parvum, Parietochloris incise, Phaeodactylum tricornutum, Crypthecodinium cohnii.

Suitable bacteria may be selected from the group of Gram positive bacteria, in particular Gram positive bacteria of the order Actinomycetales, such as Streptomyces coelicolor, Streptomyces lividans, Streptomyces albus, Streptomyces griseus, Nocardia asteroides, Nocardia corallina, Nocardia globerula, Nocardia restricta, Rhodococcus erythropolis, Rhodococcus fascians, Rhodococcus opacus, Rhodococcus ruber, Rhodococcus sp. strain 20, Mycobacterium avium, Mycobacterium ratisbonense, Mycobacterium smegmatis, Mycobacterium tuberculosis, Dietzia marls, and Gordonia amarae; Gram negative bacteria, such as Acinetobacter calcoaceticus, Acinetobacter lwoffi, Acinetobacter sp H01-N, Acinetobacter sp. 211, Pseudomonas aeruginosa; and Cyanobacteria, such as Trichodesmium erythraeum and Nostoc commune.

Suitable yeasts and fungi may be chosen from the group of Cryptococcus curvatus, Lipomyces starkeyi, Rhodosporidium toruloides, Rhodotorula glutinis, Pichia ciferii, Rhodotorula graminis, Entomophtora coronata, Cunninghamella japonica, Mortierella alpina, Mucor circinelloides, Pythium ultimum, Crypthecodinium cohnii, Schizochytrium limacinum, and Thraustochytrium aureum (for suitable yeasts and fungi, see also Ratledge C, Wynn J P. The Biochemistry and molecular biology of lipid accumulation in oleaginous microorganisms, Advances in applied microbiology (2002) 51: 1-51; see further also Qiang Hu, Milton Sommerfeld, Eric Jarvis, Maria Ghirardi, Matthew Posewitz, Michael Seibert and Al Darzins. Microalgal triacylglycerols as feedstocks for biofuel production: perspectives and advances, The Plant Journal (2008) 54, 621-639; and H. M. Alvarez, A. Steinbuechel. Triacylglycerols in prokaryotic microorganisms, Appl Microbiol Biotechnol (2002) 60:367-376, of which the contents are incorporated herein by reference).

When referred to ester or thioester of a carboxylic acid, e.g. pimelate ester or pimelate thioester, adipate ester or thioester, acetate ester of thioester, succinate ester or thioester, these terms are meant to include any activating group, in particular any biological activating group, including coenzyme A (also referred to as CoA), phospho-pantetheine, which may be bound to an acyl or peptidyl carrier protein (ACP or PCP, respectively), N-acetyl-cysteamine, methyl-thio-glycolate, methyl-mercapto-propionate, ethyl-mercapto-propionate, methyl-mercapto-butyrate, methyl-mercapto-butyrate, mercaptopropionate and other esters or thioesters providing the same or a similar function. In case living cells are used as a biocatalyst, the ester or thioester, in particular CoA, may be produced by the used biocatalyst or originate from an organism also capable of producing a suitable enzyme for catalysing the reaction. CoA-ligase and CoA-transferases have been identified in many organisms and may provide the desired activated esters or thioesters.

In an embodiment, the host cell comprises a heterologous nucleic acid sequence originating from an animal, in particular from a part thereof—e.g. liver, pancreas, brain, kidney, heart or other organ. The animal may in particular be selected from the group of mammals, more in particular selected from the group of Leporidae, Muridae, Suidae, Bovidae and Hominidae. A sequence originating from Hominidae, may in particular be from a mammal selected from the group of Homininae, more in particular from Homo sapiens. In particular if a sequence originating from Homo sapiens is used it will be used isolated from the human body.

In an embodiment, the host cell comprises a heterologous nucleic acid sequence originating from a plant. Suitable plants in particular include plants selected from the group of Asplenium; Cucurbitaceae, in particular Curcurbita, e.g. Curcurbita moschata (squash), or Cucumis; Brassicaceae, in particular Arabidopsis, e.g. A. thaliana; Mercurialis, e.g. Mercurialis perennis; Hydnocarpus; and Ceratonia.

In an embodiment, the host cell comprises a heterologous nucleic acid sequence originating from a bacterium. Suitable bacteria may in particular be selected amongst the group of Vibrio, Pseudomonas, Bacillus, Corynebacterium, Brevibacterium, Enterococcus, Streptococcus, Klebsiella, Lactococcus, Lactobacillus, Clostridium, Escherichia, Klebsiella, Anabaena, Microcystis, Synechocystis, Rhizobium, Bradyrhizobium, Thermus, Mycobacterium, Zymomonas, Proteus, Agrobacterium, Geobacillus, Acinetobacter, Azotobacter, Ralstonia, Rhodobacter, Paracoccus, Novosphingobium, Nitrosomonas, Legionella, Neisseria, Rhodopseudomonas, Staphylococcus, Deinococcus, Aerococcus and Salmonella.

In an embodiment, the host cell comprises a heterologous nucleic acid sequence originating from a fungus. Suitable fungi may in particular be selected amongst the group of Rhizopus, Phanerochaete, Emericella, Ustilago, Neurospora, Penicillium, Cephalosporium, Paecilomyces, Trichophytum and Aspergillus.

In an embodiment, the host cell comprises a heterologous nucleic acid sequence originating from a yeast. A suitable yeast may in particular be selected amongst the group of Candida, Hansenula, Kluyveromyces, Schizosaccharomyces, Pichia, Yarrowia and Saccharomyces.

It will be clear to the person skilled in the art that use can be made of a biocatalyst wherein a naturally occurring biocatalytic moiety (such as an enzyme) is expressed (wild type) or a mutant of a naturally occurring biocatalytic moiety with suitable activity in a method according to the invention. Properties of a naturally occurring biocatalytic moiety may be improved by biological techniques known to the skilled person, e.g. by molecular evolution or rational design. Mutants of wild-type biocatalytic moieties can for example be made by modifying the encoding DNA of an organism capable of producing a biocatalytic moiety (such as an enzyme) using mutagenesis techniques known to the person skilled in the art. These include random mutagenesis, site-directed mutagenesis, directed evolution, and gene recombination. In particular the DNA may be modified such that it encodes an enzyme that differs by at least one amino acid from the wild-type enzyme, so that it encodes an enzyme that comprises one or more amino acid substitutions, deletions and/or insertions compared to the wild-type, or such that the mutants combine sequences of two or more parent enzymes or by effecting the expression of the thus modified DNA in a suitable (host) cell. The latter may be achieved by methods known to the skilled person such as codon optimisation or codon pair optimisation, e.g. based on a method as described in WO 2008/000632.

A mutant biocatalyst may have improved properties, for instance with respect to one or more of the following aspects: selectivity towards the substrate, activity, stability, solvent tolerance, pH profile, temperature profile, substrate profile, susceptibility to inhibition, cofactor utilisation and substrate-affinity. Mutants with improved properties can be identified by applying e.g. suitable high through-put screening or selection methods based on such methods known to the skilled person in the art.

In accordance with a method of the invention, AKP is prepared from 2-hydroxyheptanedioic acid. The 2-hydroxyheptanedioic acid may in principle be obtained in any way. For instance 2-hydroxyheptanedioic acid may be prepared from 2-oxoheptane dioic acid or heptane dioic acid.

In a specific embodiment, 2-hydroxyheptanedioic acid is prepared by hydrolysis of a diester of 2-hydroxyheptanedioic acid. This ester can e.g. be prepared according to the following reactions.

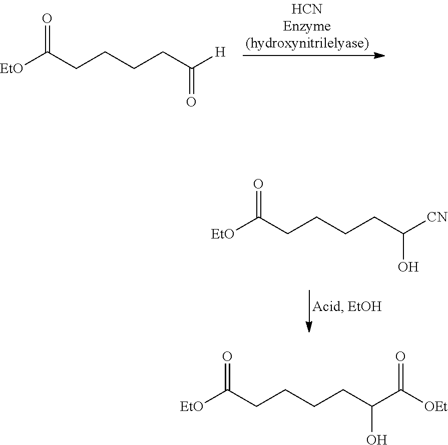

In a specific embodiment, 2-hydroxyheptanedioic acid may be obtained biocatalytically. More specifically, 2-hydroxyheptanedioic acid may be prepared from heptane dioic acid using a biocatalyst catalysing the oxidation of heptane dioic acid into 2-hydroxyheptanedioic acid. Said biocatalyst in general comprises an enzyme catalysing the oxidation of heptane dioic acid into 2-hydroxyheptanedioic acid.

In an embodiment, the enzyme catalysing this oxidation is an ‘oxidoreductase acting on paired donors (with O₂ as oxidant) and incorporation or reduction of oxygen (EC 1.14)’.

In particular such enzyme may be selected from the group of enzymes classifiable under EC 1.14.11 (with 2-oxoglutarate as one donor, and incorporation of one atom of oxygen into the other donor or into each donor), more in particular from enzymes classifyable under EC 1.14.11.1 (gamma-butyrobetaine dioxygenase), under EC 1.14.12 (with NADH or NADPH as one donor, and incorporation of two atoms of oxygen into the other donor), under EC 1.14.13 (with NADH or NADPH as one donor, and incorporation of one atom of oxygen into the other donor), under EC 1.14.14 (with reduced flavin or flavoprotein as one donor, and incorporation of one atom of oxygen into the other donor) or under EC 1.14.15 (with reduced iron-sulphur protein as one donor, and incorporation of one atom of oxygen into the other donor.

An enzyme classifyable under EC 1.14.13 may in particular be selected from the group of hydroxyphenylacteonitrile-2-monooxygenases (EC 1.14.13.42).

In a further embodiment the enzyme catalysing the oxidation of heptane dioic acid into 2-hydroxyheptanedioic acid is an oxidoreductase acting on CH or CH2 groups (EC1.17). An enzyme of EC 1.17 in a cell or for use in accordance with the invention may in particular be selected from the group of EC 1.17.1 (with NAD+ or NADP+ as acceptor), EC 1.17.3 (with oxygen as acceptor), EC 1.17.4 (with a disulphide as acceptor), EC 1.17.5 (with a quinone or similar compound as acceptor), EC 1.17.7 (with an iron-sulphur protein as acceptor), and EC 1.17.99 (with other acceptors).

In a further embodiment, the enzyme catalysing the oxidation of heptane dioic acid into 2-hydroxyheptanedioic acid is a hydroxylase with pimelate hydroxylase activity.

In a further embodiment, the enzyme catalysing the oxidation of heptane dioic acid into 2-hydroxyheptanedioic acid is a hydroxylase with pimelate-2-monooxygenase activity.

Depending on the specific enzyme the skilled person will be able to select suitable donor/acceptor systems, suitable cofactors and the like.

An enzyme catalysing the oxidation of heptane dioic acid into 2-hydroxyheptanedioic acid may in principle be selected from any organism having a nucleic acid sequence encoding such enzyme. In particular the enzyme may originate from an organism selected from the group of Corynebacterium, Escherichia (e.g. EC 1.1.3.3—malate oxidase: from Escherichia coli or an enzyme activity from E. coli referred to in the list of sequences herein below) Bacillus, Pichia, Pseudomonas, Vibrio, Zymonas, Aspergillus, Rattus (e.g. EC 1.1.1.98: (R)-2-hydroxy-fatty-acid dehydrogenases or EC 1.1.1.99: (S)-2-hydroxy-fatty-acid dehydrogenases from rat kidney), Primates (e.g. EC 1.1.1.172: 2-oxoadipate reductases from human placenta), Saccharomyces (e.g. EC 1.1.99.6: D-2-hydroxy-acid dehydrogenase or an enzyme activity from Saccharomyces referred to in the list of sequences herein below), Mirococcus (e.g. EC 1.1.3.3—malate oxidase from Micrococcus lysodeikticus), Gluconobacter, Caenorhabditis, Drosophila, Leporidae (e.g. EC 1.1.99.6: D-2-hydroxy-acid dehydrogenase from rabbit kidney)

In a specific embodiment, the enzyme catalysing the oxidation of heptane dioic acid into 2-hydroxyheptanedioic acid is selected from the group of enzymes comprising an amino acid sequence as shown Seq ID No: 191, 192, 193, 194, 195, 196, 197, 198, 199, 200, 201, 202, 203, 204, 205, 206, 207, 208, 209, 210 or a homologue of any of these sequences.

The heptane dioic acid can be obtained in any way, e.g. it can be purchased from Sigma-Aldrich, it can be prepared chemically from cyclohexanone (Organic Syntheses, Coll. Vol. 2, p. 531; Vol 11, p 42 (1931), or it can be obtained from an organism capable of synthesising pimelate. Such organism can for instance be selected from organisms capable of producing biotin via the pimeloyl-CoA pathway to biotin, e.g. E. coli, B. subtilis or B. sphaericus or other organisms mentioned herein that are capable of synthesising pimelate. The un-modified protein or gene product may be derived from genera of the Bacillus sensu lato group, Geobacillus, Brevibacillus and the like (see Table 1 in Zeigler and Perkins, 2008, Practical Handbook of Microbiology, Second Edition (E. Goldman and L. Green, eds.), pp 301-329, CRC Press, Boca Raton, Fla.) and further from genera such as Corynebacterium, Lactobacillus, Lactococci, Streptomyces (Streptomyces lydicus, Streptomyces lavendulae), and Pseudomonas. More preferably the un-modified proteins are selected from Bacillus species represented by the Bacillus sensu stricto group, in particular Bacillus subtilis, Bacillus lentimorbus, Bacillus lentus, Bacillus anthracis, Bacillus firmus, Bacillus pantothenticus, Bacillus cereus, Bacillus circulans, Bacillus coagulans, Bacillus megaterium, Bacillus thuringiensis, Bacillus licheniformis, Bacillus amyloliquefaciens, Bacillus pumilus, Bacillus halodurans (Zeigler and Perkins, 2008, Ibid). Most preferably, the un-modified proteins are selected from Bacillus subtilis 168 and its strain derivatives.

In an advantageous embodiment, a biocatalyst (used) according to the invention, comprises an enzyme system for preparing pimelate from a suitable carbon source that can be converted into pimelate, for instance by fermentation of the carbon source. In an advantageous method pimelate is prepared making use of a whole cell biotransformation of the carbon source to form pimelate. It is known that pimelate is formed from long chain fatty acids via oxidative cleavage. Such fatty acids may therefore be provided as a as carbon source, e.g. by supplying plant oils, fatty acid esters (bio-diesel) or the like to a biocatalyst (in particular in case it is a host cell) in a method of the invention. For instance a host cell may be selected naturally comprising such system—such as E. coli or B. sphaericus—or the host cell may be obtained by genetic modification. For instance a host cell may be provided with at least one gene selected from bioC and bioH (from E. coli) or at least one gene selected from bioI, bioW, bioX and bioH (see also W. R. Streit, P. Entcheva. Biotin in microbes, the genes involved in its biosynthesis, its biochemical role and perspectives for biotechnological production. Appl Microbiol Biotechnol (2003) 61:21-31).

The carbon source may in particular contain at least one compound selected from the group of monohydric alcohols, polyhydric alcohols, carboxylic acids, carbon dioxide, fatty acids, glycerides, tri- and di-acyl-glycerides including mixtures comprising any of said compounds. Suitable monohydric alcohols include methanol and ethanol, Suitable polyols include glycerol and carbohydrates. Suitable fatty acids or glycerides may in particular be provided in the form of an edible oil, preferably of plant origin.

In particular a carbohydrate may be used, because usually carbohydrates can be obtained in large amounts from a biologically renewable source, such as an agricultural product, preferably an agricultural waste-material. Preferably a carbohydrate is used selected from the group of glucose, fructose, sucrose, lactose, saccharose, starch, cellulose and hemi-cellulose. Particularly preferred are glucose, oligosaccharides comprising glucose and polysaccharides comprising glucose and hydrolysates of said oligosaccharides or said polysaccharides.

In accordance with a method according to the invention 2-hydroxyheptanedioic acid is biocatalytically converted into AKP. The biocatalyst may in particular comprise an enzyme for catalysing the conversion of hydroxyheptanedioic acid into AKP selected from the group of
oxidoreductases acting on the CH—OH group of donors (EC 1.1), in particular such an oxidoreductase selected from the group of EC 1.1.1 (with NAD+ or NADP+ as acceptor), EC 1.1.2 (with a cytochrome as acceptor), EC 1.1.3 (with oxygen as acceptor), EC 1.1.4 (with a disulphide as acceptor), EC 1.1.5 (with a quinone or similar compound as acceptor), EC 1.1.7 (with an iron sulphur protein as acceptor), and EC 1.1.99 (with other acceptors);
oxidoreductases acting on the aldehyde or oxo group of donors (EC 1.2);
enzymes with 2-hydroxypimelate dehydrogenase activity enzymes with 2-hydroxypimelate oxidase activity;
oxidoreductases classified under EC 1.97; and
oxidoreductases classified under EC 1.98.

An oxidoreductase classifiable under EC 1.1.1 catalysing the conversion of hydroxyheptanedioic acid into AKP may in particular be selected from alcohol dehydrogenases with NAD+ as acceptor of EC 1.1.1.1; alcohol dehydrogenases with NADP+ as acceptor of EC 1.1.1.2; glyoxylate reductases of EC 1.1.1.26, L-lactate dehydrogenases of EC 1.1.1.27, D-lactate dehydrogenases of EC 1.1.1.28, glycerate dehydrogenases of EC 1.1.1.29, 3-hydroxybutyrate dehydrogenases of EC 1.1.1.30, 3-hydroxyisobutyrate dehydrogenases of EC 1.1.1.31, malate dehydrogenase of EC 1.1.1.37, 3-hydroxypropionate dehydrogenase of EC 1.1.1.59, 2-hydroxy-3-oxopropionate reductase of EC 1.1.1.60, alcohol dehydrogenase [NAD(P)+] of EC 1.1.1.71, glyoxylate reductase [NADP+] of EC 1.1.1.79, hydroxypyruvate reductases of EC 1.1.1.81, malate dehydrogenases [NADP+] of EC 1.1.1.82, 3-isopropylmalate dehydrogenases of EC 1.1.1.85, tartrate dehydrogenases of EC 1.1.1.93, (R)-2-hydroxy-fatty-acid dehydrogenases of EC 1.1.1.98, (S)-2-hydroxy-fatty-acid dehydrogenases of EC 1.1.1.99, hydroxymalonate dehydrogenase of EC 1.1.1.167, 2-oxoadipate reductases of EC 1.1.1.172, hydroxyphenylpyruvate reductases of EC 1.1.1.237, and 3-hydroxypimeloyl-CoA dehydrogenases of EC 1.1.1.259

An enzyme classifiable under EC 1.1.2 catalysing the conversion of hydroxyheptanedioic acid into AKP may in particular be selected from D-lactate dehydrogenases (EC 1.1.2.4 and EC 1.1.2.5).

An enzyme classifiable under EC 1.1.3 catalysing the conversion of hydroxyheptanedioic acid into AKP may in particular be selected from the group of lactate oxidases and other hydroxy acid oxidases; malate oxidases (EC 1.1.3.3), (S)-2-hydroxy-acid oxidase (EC 1.1.3.15); secondary-alcohol oxidases (EC 1.1.3.18); hydroxyphytanate oxidases (EC 1.1.3.27).

An enzyme classifiable under EC 1.1.99 catalysing the conversion of hydroxyheptanedioic acid into AKP may in particular be selected from 2-hydroxyglutarate dehydrogenases (EC 1.1.99.2); D-2-hydroxy-acid dehydrogenases (EC 1.1.99.6); glycolate dehydrogenase (EC 1.1.99.14), malate dehydrogenase (EC 1.1.99.16), and 2-oxo-acid reductases (EC 1.1.99.30).

In a particularly preferred method, an enzyme catalysing the preparation of AKP is selected from the group of
oxidoreductases with oxygen as acceptor (EC 1.1.3), such as a lactate oxidase or another hydroxy acid oxidase; such as hydroxy acid oxidase HAO1 from Homimidae, in particular from Homo sapiens (EC 1.1.3.15) or lactate oxidase from Aerococci, in particular from Aerococcus viridans;
L-lactate dehydrogenases (EC 1.1.1.27);
D-lactate dehydrogenases (EC 1.1.1.28);
malate dehydrogenase [NAD+] (EC 1.1.1.37);
hydroxypyruvate reductases (EC1.1.1.81);
malate dehydrogenases [NADP+] (EC 1.1.1.82);
3-isopropylmalate dehydrogenases (EC 1.1.1.85);
tartrate dehydrogenases (EC 1.1.1.93);
(R)-2-hydroxy-fatty-acid dehydrogenases (EC1.1.1.98);
(S)-2-hydroxy-fatty-acid dehydrogenases (EC 1.1.1.99);
2-oxoadipate reductases (EC1.1.1.172);
2-hydroxyglutarate dehydrogenase (EC 1.1.99.2); and
D-2-hydroxy-acid dehydrogenase (EC 1.1.99.6).

Most preferably, the enzyme catalysing the preparation of AKP is selected from the group of 2-oxoadipate reductases (EC1.1.1.172).

In a specifically preferred the enzyme comprises an amino acid sequence according to SEQ ID NO: 186, SEQ ID NO: 189, or a homologue of any of these sequences. Suitable nucleic acids encoding an enzyme catalysing the preparation of AKP may in particular comprise a nucleic acid sequence represented by SEQ ID NO: 185, SEQ ID NO: 187, SEQ ID NO: 188, SEQ ID NO: 190 and functional analogues thereof.

In a specific embodiment, AKP prepared in accordance with the invention is used for the preparation of 6-ACA. The inventors have realised that AKP can be converted into 6-ACA by a method wherein first AKP is decarboxylated to form 5-FVA after which 6-ACA can be prepared from 5-FVA using an amino transfer reaction or wherein first AKP is subjected to an amino transfer reaction to form AAP, after which 6-ACA can be prepared from AAP by a decarboxylation reaction.

In a preferred method for preparing 6-ACA, the preparation comprises a biocatalytic reaction in the presence of a biocatalyst capable of catalysing the decarboxylation of an alpha-keto acid or an amino acid (i.e. a compound comprising at least one carboxylic acid group and at least one amino group). An enzyme having such catalytic activity may therefore be referred to as an alpha-keto acid decarboxylase respectively an amino acid decarboxylase.

Said acid preferably is a diacid, wherein the said biocatalyst is selective towards the acid group next to the keto- or amino-group.

In general, a suitable decarboxylase has alpha-ketopimelate decarboxylase activity, capable of catalysing the conversion of AKP into 5-FVA or alpha-aminopimelate decarboxylase activity, capable of catalysing the conversion of AAP to 6-ACA.

An enzyme capable of decarboxylating an alpha-keto acid or an amino acid may in particular be selected from the group of decarboxylases (E.C. 4.1.1), preferably from the group of glutamate decarboxylases (EC 4.1.1.15), diaminopimelate decarboxylases (EC 4.1.1.20), aspartate 1-decarboxylases (EC 4.1.1.11), branched chain alpha-keto acid decarboxylases, alpha-ketoisovalerate decarboxylases, alpha-ketoglutarate decarboxylases, and pyruvate decarboxylases (EC 4.1.1.1).

One or more other suitable decarboxylases may in particular be selected amongst the group of oxalate decarboxylases (EC 4.1.1.2), oxaloacetate decarboxylases (EC 4.1.1.3), acetoacetate decarboxylases (EC 4.1.1.4), valine decarboxylases/leucine decarboxylases (EC 4.1.1.14), 3-hydroxyglutamate decarboxylases (EC 4.1.1.16), ornithine decarboxylases (EC 4.1.1.17), lysine decarboxylases (EC 4.1.1.18), arginine decarboxylases (EC 4.1.1.19), 2-oxoglutarate decarboxylases (EC 4.1.1.71), and diaminobutyrate decarboxylases (EC 4.1.1.86)

A decarboxylase may in particular be a decarboxylase of an organism selected from the group of squashes; cucumbers; yeasts; fungi, e.g. Saccharomyces cerevisiae, Candida flareri, Hansenula sp., Kluyveromyces marxianus, Rhizopus javanicus, Zymomonas mobilis, more in particular pyruvate decarboxylase mutant 1472A from Zymomonas mobilis, and Neurospora crassa; mammals, in particular from mammalian brain; and bacteria. For instance glutamate decarboxylase, aspartate decarboxylase, alpha-keto-isovalerate decarboxylase and branched chain alpha-keto acid decarboxylase from Eschericia coli (E. coli) may be used, or glutamate decarboxylase from Neurospora crassa, Mycobacterium leprae, Clostridium perfringens, Lactobacillus brevis, Mycobacterium tuberculosis, Streptococcus or Lactococcus may be used. Examples of Lactococcus species from which the glutamate decarboxylase may originate in particular include Lactococcus lactis, such as Lactococcus lactis strain B1157, Lactococcus lactis IFPL730, more in particular Lactococcus lactis var. maltigenes (formerly named Streptococcus lactis var. maltigenes). An oxaloacetate decarboxylase from Pseudomonas may in particular be used.

Specific examples of decarboxylases that may be used and genes encoding such decarboxylases are shown in Sequence ID No's: 105-122.

In a preferred method of the invention, the preparation of 6-ACA comprises an enzymatic reaction in the presence of an enzyme capable of catalysing a transamination reaction in the presence of an amino donor, selected from the group of aminotransferases (E.C. 2.6.1).

In general, a suitable aminotransferase has 6-aminocaproic acid 6-aminotransferase activity, capable of catalysing the conversion of 5-FVA into 6-ACA op alpha-aminopimelate 2-aminotransferase activity, capable of catalysing the conversion of AKP into AAP.

The aminotransferase may in particular be selected amongst the group of β-aminoisobutyrate: alpha-ketoglutarate aminotransferases, β-alanine aminotransferases, aspartate aminotransferases, 4-amino-butyrate aminotransferases (EC 2.6.1.19), L-lysine 6-aminotransferase (EC 2.6.1.36), 2-aminoadipate aminotransferases (EC 2.6.1.39), 5-aminovalerate aminotransferases (EC 2.6.1.48), 2-aminohexanoate aminotransferases (EC 2.6.1.67) and lysine:pyruvate 6-aminotransferases (EC 2.6.1.71).

In an embodiment an aminotransferase may be selected amongst the group of alanine aminotransferases (EC 2.6.1.2), leucine aminotransferases (EC 2.6.1.6), alanine-oxo-acid aminotransferases (EC 2.6.1.12), β-alanine-pyruvate aminotransferases (EC 2.6.1.18), (S)-3-amino-2-methylpropionate aminotransferases (EC 2.6.1.22), L,L-diaminopimelate aminotransferase (EC 2.6.1.83).

The aminotransferase may in particular be selected amongst aminotransferases from Vibrio, in particular Vibrio fluvialis; Pseudomonas, in particular Pseudomonas aeruginosa; Bacillus, in particular Bacillus weihenstephanensis; Mercurialis, in particular Mercurialis perennis, more in particular shoots of Mercurialis perennis; Asplenium, more in particular Asplenium unilaterale or Asplenium septentrionale; Ceratonia, more in particular Ceratonia siliqua; a mammal; or yeast, in particular Saccharomyces cerevisiae. In case the enzyme is of a mammal, it may in particular originate from mammalian kidney, from mammalian liver, from mammalian heart or from mammalian brain. For instance a suitable enzyme may be selected amongst the group of β-aminoisobutyrate: alpha-ketoglutarate aminotransferase from mammalian kidney, in particular β-aminoisobutyrate: alpha-ketoglutarate aminotransferase from hog kidney; β-alanine aminotransferase from mammalian liver, in particular β-alanine aminotransferase from rabbit liver; aspartate aminotransferase from mammalian heart; in particular aspartate aminotransferase from pig heart; 4-amino-butyrate aminotransferase from mammalian liver, in particular 4-amino-butyrate aminotransferase from pig liver; 4-amino-butyrate aminotransferase from mammalian brain, in particular 4-aminobutyrate aminotransferase from human, pig, or rat brain.

In an embodiment the aminotransferase is selected from the group of alpha-ketoadipate-glutamate aminotransferase from Neurospora, in particular alpha-ketoadipate:glutamate aminotransferase from Neurospora crassa; 4-amino-butyrate aminotransferase from E. coli, or alpha-aminoadipate aminotransferase from Thermus, in particular alpha-aminoadipate aminotransferase from Thermus thermophilus, and 5-aminovalerate aminotransferase from Clostridium in particular from Clostridium aminovalericum. A suitable 2-aminoadipate aminotransferase may e.g. be provided by Pyrobaculum islandicum.

In a specific embodiment, an aminotransferase is used comprising an amino acid sequence according to SEQ ID NO: 2, 83, 86, 90, 92, 94, 96, 98, 100, 102, 104, or a homologue of this sequence. Suitable nucleic acid sequences encoding such an aminotransferase include the sequences of SEQ ID NO: 1, 82, 84, 85, 89, 91, 93, 95, 97, 99, 101, and 103. Further Sequence ID NO: 3 represents a codon optimised nucleic acid sequence for the amino acid sequence according to SEQ ID NO: 2.

In particular, the amino donor can be ammonia, ammonium ion, an amine or an amino acid. Suitable amines are primary amines and secondary amines. The amino acid may have a D- or L-configuration. Examples of amino donors are alanine, glutamate, isopropylamine, 2-aminobutane, 2-aminoheptane, phenylmethanamine, 1-phenyl-1-aminoethane, glutamine, tyrosine, phenylalanine, aspartate, β-aminoisobutyrate, β-alanine, 4-aminobutyrate, and alpha-aminoadipate.

In a further preferred embodiment, the method for preparing 6-ACA comprises a biocatalytic reaction in the presence of an enzyme capable of catalysing a reductive amination reaction in the presence of an ammonia source, selected from the group of oxidoreductases acting on the CH—NH₂ group of donors (EC 1.4), in particular from the group of amino acid dehydrogenases (E.C. 1.4.1). In general, a suitable amino acid dehydrogenase has 6-aminocaproic acid 6-dehydrogenase activity, catalysing the conversion of 5-FVA into 6-ACA or has alpha-aminopimelate 2-dehydrogenase activity, catalysing the conversion of AKP into AAP. In particular a suitable amino acid dehydrogenase be selected amongst the group of diaminopimelate dehydrogenases (EC 1.4.1.16), lysine 6-dehydrogenases (EC 1.4.1.18), glutamate dehydrogenases (EC 1.4.1.3; EC 1.4.1.4), and leucine dehydrogenases (EC 1.4.1.9).

In an embodiment, an amino acid dehydrogenase may be selected amongst an amino acid dehydrogenases classified as glutamate dehydrogenases acting with NAD or NADP as acceptor (EC 1.4.1.3), glutamate dehydrogenases acting with NADP as acceptor (EC 1.4.1.4), leucine dehydrogenases (EC 1.4.1.9), diaminopimelate dehydrogenases (EC 1.4.1.16), and lysine 6-dehydrogenases (EC 1.4.1.18).

An amino acid dehydrogenase may in particular originate from an organism selected from the group of Corynebacterium, in particular Corynebacterium glutamicum; Proteus, in particular Proteus vulgaris; Agrobacterium, in particular Agrobacterium tumefaciens; Geobacillus, in particular Geobacillus stearothermophilus; Acinetobacter, in particular Acinetobacter sp. ADP1; Raistonia, in particular Raistonia solanacearum; Salmonella, in particular Salmonella typhimurium; Saccharomyces, in particular Saccharomyces cerevisiae; Brevibacterium, in particular Brevibacterium flavum; and Bacillus, in particular Bacillus sphaericus, Bacillus cereus or Bacillus subtilis. For instance a suitable amino acid dehydrogenase may be selected amongst diaminopimelate dehydrogenases from Bacillus, in particular Bacillus sphaericus; diaminopimelate dehydrogenases from Brevibacterium sp.; diaminopimelate dehydrogenases from Corynebacterium, in particular diaminopimelate dehydrogenases from Corynebacterium glutamicum; diaminopimelate dehydrogenases from Proteus, in particular diaminopimelate dehydrogenase from Proteus vulgaris; lysine 6-dehydrogenases from Agrobacterium, in particular Agrobacterium tumefaciens, lysine 6-dehydrogenases from Geobacillus, in particular from Geobacillus stearothermophilus; glutamate dehydrogenases acting with NADH or NADPH as cofactor (EC 1.4.1.3) from Acinetobacter, in particular glutamate dehydrogenases from Acinetobacter sp. ADP1; glutamate dehydrogenases (EC 1.4.1.3) from Ralstonia, in particular glutamate dehydrogenases from Ralstonia solanacearum; glutamate dehydrogenases acting with NADPH as cofactor (EC 1.4.1.4) from Salmonella, in particular glutamate dehydrogenases from Salmonella typhimurium; glutamate dehydrogenases (EC 1.4.1.4) from Saccharomyces, in particular glutamate dehydrogenases from Saccharomyces cerevisiae; glutamate dehydrogenases (EC 1.4.1.4) from Brevibacterium, in particular glutamate dehydrogenases from Brevibacterium flavum; and leucine dehydrogenases from Bacillus, in particular leucine dehydrogenases from Bacillus cereus or Bacillus subtilis.

In a specific embodiment, AKP is biocatalytically converted into 5-formylpentanoate (5-FVA) in the presence of a decarboxylase or other biocatalyst catalysing such conversion. A decarboxylase used in accordance with the invention may in particular be selected from the group of alpha-keto acid decarboxylases from E. coli, Lactococcus lactis, Lactococcus lactis var. maltigenes or Lactococcus lactis subsp. cremoris; branched chain alpha-keto acid decarboxylases from E. coli, Lactococcus lactis strain B1157 or Lactococcus lactis IFPL730; pyruvate decarboxylases from Saccharomyces cerevisiae, Candida flareri, Zymomonas mobilis, Hansenula sp., Rhizopus javanicus, Neurospora crassa, or Kluyveromyces marxianus; α-ketoglutarate decarboxylases from Mycobacterium tuberculosis; glutamate decarboxylases from E. coli, Lactobacillus brevis, Mycobacterium leprae, Neurospora crassa or Clostridium perfringens; and aspartate decarboxylases from E. coli.

Thereafter 5-FVA may be converted into 6-ACA. This can be done chemically: 6-ACA can be prepared in high yield by reductive amination of 5-FVA with ammonia over a hydrogenation catalyst, for example Ni on SiO₂/Al₂O₃ support, as described for 9-aminononanoic acid (9-aminopelargonic acid) and 12-aminododecanoic acid (12-aminolauric acid) in EP-A 628 535 or DE 4 322 065.

Alternatively, 6-ACA can be obtained by hydrogenation over PtO₂ of 6-oximocaproic acid, prepared by reaction of 5-FVA and hydroxylamine. (see e.g. F. O. Ayorinde, E. Y. Nana, P. D. Nicely, A. S. Woods, E. O. Price, C. P. Nwaonicha J. Am. Oil Chem. Soc. 1997, 74, 531-538 for synthesis of the homologous 12-aminododecanoic acid).

In an embodiment, the conversion of 5-FVA to 6-ACA may be performed biocatalytically in the presence of (i) an amino donor and (ii) an aminotransferase, an amino acid dehydrogenase or another biocatalyst capable of catalysing such conversion. In particular in such an embodiment the aminotransferase may be selected from the group of aminotransferases from Vibrio fluvialis, Pseudomonas aeruginosa or Bacillus weihenstephanensis; β-aminoisobutyrate:αλπηα-ketoglutarate aminotransferase from hog kidney; β-alanine aminotransferase from rabbit liver; aminotransferase from shoots from Mercurialis perennis; 4-aminobutyrate aminotransferase from pig liver or from human, rat, or pig brain; β-alanine aminotransferase from rabbit liver; and Llysine:alpha-ketoglutarate-ε-aminotransferase. In case an amino acid dehydrogenase is used, such amino acid dehydrogenase may in particular be selected from the group of lysine 6-dehydrogenases from Agrobacterium tumefaciens or Geobacillus stearothermophilus. Another suitable amino acid dehydrogenase may be selected from the group of diaminopimelate dehydrogenases from Bacillus sphaericus, Brevibacterium sp., Corynebacterium glutamicum, or Proteus vulgaris; from the group of glutamate dehydrogenases acting with NADH or NADPH as cofactor (EC 1.4.1.3) from Acinetobacter sp. ADP1 or Ralstonia solanacearum; from the group of glutamate dehydrogenases acting with NADPH as cofactor (EC 1.4.1.4) from Salmonella typhimurium; from the group of glutamate dehydrogenases (EC 1.4.1.4) from Saccharomyces cerevisiae or Brevibacterium flavum; or from the group of leucine dehydrogenases from Bacillus cereus or Bacillus subtilis.

In a specific embodiment, AKP is chemically converted into 5-FVA. Efficient chemical decarboxylation of 2-keto carboxylic acid into the corresponding aldehyde can be performed by intermediate enamine formation using a secondary amine, for instance morpholine, under azeotropic water removal and simultaneous loss of CO₂, e.g. based on a method as described in Tetrahedron Lett. 1982, 23(4), 459-462. The intermediate terminal enamide is subsequently hydrolysed to the corresponding aldehyde. 5-FVA may thereafter be biocatalytically converted into 6-ACA by transamination in the presence of an aminotransferase or by enzymatic reductive amination by an amino acid dehydrogenase or another biocatalyst able of catalysing such conversion. Such aminotransferase or amino acid dehydrogenase may in particular be selected from the biocatalysts mentioned above when describing the conversion of 5-FVA to 6-ACA.

Alternatively, the conversion of 5-FVA to 6-ACA may be performed by a chemical method, e.g. as mentioned above.

In a specific embodiment, AKP is biocatalytically converted into AAP in the presence of (i) an aminotransferase, an amino acid dehydrogenase, or another biocatalyst capable of catalysing such conversion and (ii) an amino donor. Such aminotransferase used in accordance with the invention for the conversion of AKP to AAP may in particular be selected from the group of aspartate aminotransferases from pig heart; alpha-ketoadipate:glutamate aminotransferases from Neurospora crassa or yeast; aminotransferases from shoots from Mercurialis perennis; 4-aminobutyrate aminotransferases from E. coli; alpha-aminoadipate aminotransferases from Thermus thermophilus; aminotransferases from Asplenium septentrionale or Asplenium unilaterale; and aminotransferases from Ceratonia siliqua.

Suitable amino acid dehydrogenases may in particular be selected amongst the group of glutamate dehydrogenases acting with NADH or NADPH as cofactor (EC 1.4.1.3) from Acinetobacter sp. ADP1 or Ralstonia solanacearum; glutamate dehydrogenases acting with NADPH as cofactor (EC 1.4.1.4) from Salmonella typhimurium, Saccharomyces cerevisiae, or Brevibacterium flavum; aminopimelate dehydrogenases from Bacillus sphaericus, Brevibacterium sp., Corynebacterium glutamicum, or Proteus vulgaris. Another suitable amino acid dehydrogenase may be selected from the group of lysine 6-dehydrogenases from Agrobacterium tumefaciens or Geobacillus stearothermophilus; or from the group of leucine dehydrogenases from Bacillus cereus or Bacillus subtilis.

Thereafter AAP may be chemically converted to 6-ACA by decarboxylation. This can be performed by heating in a high boiling solvent in the presence of a ketone or aldehyde catalyst. For example, amino acids are decarboxylated in good yields in cyclohexanol at 150-160° C. with 1-2 v/v % of cyclohexenone as described by M. Hashimoto, Y. Eda, Y. Osanai, T. Iwai and S. Aoki in Chem. Lett. 1986, 893-896. Similar methods are described in Eur. Pat. Appl. 1586553, 2005 by Daiso, and by S. D. Brandt, D. Mansell, S. Freeman, I. A. Fleet, J. F. Alder J. Pharm. Biomed. Anal. 2006, 41, 872-882.

Alternatively, the decarboxylation of AAP to 6-ACA may be performed biocatalytically in the presence of a decarboxylase or other biocatalyst catalysing such decarboxylation. The decarboxylase may be selected amongst decarboxylases capable of catalysing the decarboxylation of an alpha-amino acid. In particular, the decarboxylase may be selected from the group of glutamate decarboxylases from Curcurbita moschata, cucumber, yeast, or calf brain; and diaminopimelate decarboxylases (EC 4.1.1.20). A diaminopimelate decarboxylase may, e.g., be from an organism capable of synthesising lysine from diaminopimelate. Such organism may in particular be found amongst bacteria, archaea and plants. In particular, the diaminopimelate decarboxylase may be from a gram negative bacterium, for instance E. coli.

In a specific embodiment, AKP is chemically converted into AAP. AAP can be prepared from 2-oxopimelic acid by catalytic Leuckart-Wallach reaction as described for similar compounds. This reaction is performed with ammonium formate in methanol and [RhCp*Cl₂]₂ as homogeneous catalyst (M. Kitamura, D. Lee, S. Hayashi, S. Tanaka, M. Yoshimura J. Org. Chem. 2002, 67, 8685-8687). Alternatively, the Leuckart-Wallach reaction can be performed with aqueous ammonium formate using [Ir^{III}Cp*(bpy)H₂O]SO₄ as catalyst as described by S. Ogo, K. Uehara and S. Fukuzumi in J. Am. Chem. Soc. 2004, 126, 3020-3021. Transformation of αλπηα-keto acids into (enantiomerically enriched) amino acids is also possible by reaction with (chiral) benzylamines and subsequent hydrogenation of the intermediate imine over Pd/C or Pd(OH)₂/C. See for example, R. G. Hiskey, R. C. Northrop J. Am. Chem. Soc. 1961, 83, 4798.

Thereafter AAP may be biocatalytically converted into 6-ACA, in the presence of a decarboxylase or another biocatalyst capable of performing such decarboxylation. Such decarboxylase may in particular be selected amongst the biocatalysts referred to above, when describing biocatalysts for the conversion of AAP to 6-ACA.

Alternatively, the conversion of AAP to 6-ACA may be performed by a chemical method, e.g. as mentioned above.

In a specific embodiment, AKP is biocatalytically converted into 5-FVA in the presence of a decarboxylase or other biocatalyst capable of catalysing such conversion and 5-FVA is thereafter converted into 6-ACA in the presence of an aminotransferase, amino acid dehydrogenase, or other biocatalyst capable of catalysing such conversion. Decarboxylases suitable for these reactions may in particular be selected from the group of decarboxylases mentioned above, when describing the biocatalytic conversion of AKP into 5-FVA. A suitable aminotransferase or amino acid dehydrogenase for the conversion of 5-FVA may in particular be selected from those mentioned above, when describing the biocatalytic conversion of 5-FVA to 6-ACA.

In a specific embodiment, AKP is biocatalytically converted into AAP in the presence of an aminotransferase, amino acid dehydrogenase, or other biocatalyst capable of catalysing such conversion and AAP is thereafter converted into 6-ACA in the presence of a decarboxylase. Enzymes suitable for these reactions may in particular be selected from the group of aminotransferases, amino acid dehydrogenases, and decarboxylases which have been described above when describing the biocatalytic conversion of AKP into AAP and the biocatalytic conversion of AAP into 6-ACA respectively.

In another embodiment of the invention, 5-FVA—prepared from AKP made in a method according to the invention—is converted into adipic acid by oxidation of the aldehyde group. This may be accomplished chemically, e.g. by selective chemical oxidation or biocatalytically. In a preferred method of the invention, the preparation comprises a biocatalytic reaction in the presence of a biocatalyst capable of catalysing the oxidation of an aldehyde group. The biocatalyst may use NAD or NADP as cofactor.

An enzyme having catalytic activity in the oxidation of an aldehyde group may in particular be selected from the group of oxidoreductases (EC 1.2.1), preferably from the group of aldehyde dehydrogenase (EC 1.2.1.3, EC 1.2.1.4 and EC 1.2.1.5), malonate-semialdehyde dehydrogenase (EC 1.2.1.15), succinate-semialdehyde dehydrogenase (EC 1.2.1.16 and EC 1.2.1.24); glutarate-semialdehyde dehydrogenase (EC 1.2.1.20), aminoadipate semialdehyde dehydrogenase (EC 1.2.1.31), adipate semialdehyde dehydrogenase (EC 1.2.1.63). Adipate semialdehyde dehydrogenase activity has been described, for example, in the caprolactam degradation pathway in the KEGG database.

An aldehyde dehydrogenase may in principle be obtained or derived from any organism. The organism may be prokaryotic or eukaryotic. In particular the organism can be selected from bacteria, archaea, yeasts, fungi, protists, plants and animals (including human).

In an embodiment the bacterium is selected from the group of Acinetobacter (in particular Acinetobacter baumanii and Acinetobacter sp. NCIMB9871), Azospirillum (in particular Azospirillum brasilense) Raistonia, Bordetella, Burkholderia, Methylobacterium, Xanthobacter, Sinorhizobium, Rhizobium, Nitrobacter, Brucella (in particular B. melitensis), Pseudomonas, Agrobacterium (in particular Agrobacterium tumefaciens), Bacillus, Listeria, Alcaligenes, Corynebacterium, and Flavobacterium.

In an embodiment the organism is selected from the group of yeasts and fungi, in particular from the group of Aspergillus (in particular A. niger and A. nidulans) and Penicillium (in particular P. chrysogenum).

In an embodiment, the organism is a plant, in particular Arabidopsis, more in particular A. thaliana.

In a specific embodiment, the biocatalyst comprises an enzyme (having catalytic activity in the oxidation of an aldehyde group) represented by Sequence ID 78-81 or a homologue thereof.

In another embodiment of the invention, 6-ACA—prepared from AKP made in a method according to the invention—is converted into diaminohexane. This may be accomplished by reducing the acid group to form an aldehyde group, and transaminating the thus formed aldehyde group, thereby providing an aminogroup, yielding diaminohexane. This may be accomplished chemically or biocatalytically. In a preferred method of the invention, the preparation comprises a biocatalytic reaction in the presence of a biocatalyst capable of catalysing the reduction of the acid to form an aldehyde group and/or a biocatalytic reaction in the presence of a biocatalyst capable of catalysing said transamination, in the presence of an amino donor, e.g. an amino donor as described elsewhere herein.

A biocatalyst capable of catalysing the reduction of the acid group to form an aldehyde group may in particular comprise an enzyme selected from the group of oxidoreductases (EC 1.2.1), preferably from the group of aldehyde dehydrogenases (EC 1.2.1.3, EC 1.2.1.4 and EC 1.2.1.5), e.g. found in an organism as described elsewhere herein. A biocatalyst capable of catalysing said transamination may in particular comprise an enzyme selected from the group of aminotransferases (E.C. 2.6.1), e.g. found in an organism as described elsewhere herein.

The product obtained in a method according to the invention (such as AKP, 6-ACA) can be isolated from the biocatalyst, as desired. A suitable isolation method can be based on methodology commonly known in the art.

Reaction conditions in a method of the invention may be chosen depending upon known conditions for the biocatalyst, in particular the enzyme, the information disclosed herein and optionally some routine experimentation.

In principle, the pH of the reaction medium used may be chosen within wide limits, as long as the biocatalyst is active under the pH conditions. Alkaline, neutral or acidic conditions may be used, depending on the biocatalyst and other factors. In case the method includes the use of a micro-organism, e.g. for expressing an enzyme catalysing a method of the invention, the pH is selected such that the micro-organism is capable of performing its intended function or functions. The pH may in particular be chosen within the range of four pH units below neutral pH and two pH units above neutral pH, i.e. between pH 3 and pH 9 in case of an essentially aqueous system at 25° C. A system is considered aqueous if water is the only solvent or the predominant solvent (>50 wt. %, in particular >90 wt. %, based on total liquids), wherein e.g. a minor amount (<50 wt. %, in particular <10 wt. %, based on total liquids) of alcohol or another solvent may be dissolved (e.g. as a carbon source) in such a concentration that micro-organisms which may be present remain active. In particular in case a yeast and/or a fungus is used, acidic conditions may be preferred, in particular the pH may be in the range of pH 3 to pH 8, based on an essentially aqueous system at 25° C. If desired, the pH may be adjusted using an acid and/or a base or buffered with a suitable combination of an acid and a base.

In principle, the incubation conditions can be chosen within wide limits as long as the biocatalyst shows sufficient activity and/or growth. This includes aerobic, micro-aerobic, oxygen limited and anaerobic conditions.

Anaerobic conditions are herein defined as conditions without any oxygen or in which substantially no oxygen is consumed by the biocatalyst, in particular a micro-organism, and usually corresponds to an oxygen consumption of less than 5 mmol/l·h, in particular to an oxygen consumption of less than 2.5 mmol/l·h, or less than 1 mmol/l·h.

Aerobic conditions are conditions in which a sufficient level of oxygen for unrestricted growth is dissolved in the medium, able to support a rate of oxygen consumption of at least 10 mmol/l·h, more preferably more than 20 mmol/l·h, even more preferably more than 50 mmol/l·h, and most preferably more than 100 mmol/l·h.

Oxygen-limited conditions are defined as conditions in which the oxygen consumption is limited by the oxygen transfer from the gas to the liquid. The lower limit for oxygen-limited conditions is determined by the upper limit for anaerobic conditions, i.e. usually at least 1 mmol/l·h, and in particular at least 2.5 mmol/l·h, or at least 5 mmol/l·h. The upper limit for oxygen-limited conditions is determined by the lower limit for aerobic conditions, i.e. less than 100 mmol/l·h, less than 50 mmol/l·h, less than 20 mmol/l·h, or less than to 10 mmol/l·h.

Whether conditions are aerobic, anaerobic or oxygen limited is dependent on the conditions under which the method is carried out, in particular by the amount and composition of ingoing gas flow, the actual mixing/mass transfer properties of the equipment used, the type of micro-organism used and the micro-organism density.

In a preferred method of the invention, at least the preparation of AKP is carried out under fermentative conditions. The term fermentative conditions is used herein in a broad sense, as is common in the art, i.e. it is used to refer to industrial methods wherein a micro-organism is used to prepare a product of interest. Such methods under fermentative conditions can be carried out in an aerobic, anaerobic or oxygen limited environment. The term may be used to distinguish a method from biocatalytic methods wherein one or more enzymes are used, isolated from the organism in which the enzyme has been expressed.

In principle, the temperature used is not critical, as long as the biocatalyst, in particular the enzyme, shows substantial activity. Generally, the temperature may be at least 0° C., in particular at least 15° C., more in particular at least 20° C. A desired maximum temperature depends upon the biocatalyst. In general such maximum temperature is known in the art, e.g. indicated in a product data sheet in case of a commercially available biocatalyst, or can be determined routinely based on common general knowledge and the information disclosed herein. The temperature is usually 90° C. or less, preferably 70° C. or less, in particular 50° C. or less, more in particular or 40° C. or less.

In particular if a biocatalytic reaction is performed outside a host organism, a reaction medium comprising an organic solvent may be used in a high concentration (e.g. more than 50%, or more than 90 wt. %), in case an enzyme is used that retains sufficient activity in such a medium.

A heterologous cell comprising one or more enzymes for catalysing a reaction step in a method of the invention can be constructed using molecular biological techniques, which are known in the art per se. For instance, such techniques can be used to provide a vector which comprises one or more genes encoding one or more of said biocatalysts. A vector comprising one or more of such genes can comprise one or more regulatory elements, e.g. one or more promoters, which may be operably linked to a gene encoding an biocatalyst.

As used herein, the term “operably linked” refers to a linkage of polynucleotide elements (or coding sequences or nucleic acid sequence) in a functional relationship. A nucleic acid sequence is “operably linked” when it is placed into a functional relationship with another nucleic acid sequence. For instance, a promoter or enhancer is operably linked to a coding sequence if it affects the transcription of the coding sequence.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skilled in the art to act directly or indirectly to regulate the amount of transcription from the promoter. A “constitutive” promoter is a promoter that is active under most environmental and developmental conditions. An “inducible” promoter is a promoter that is active under environmental or developmental regulation. The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain.

The promoter that could be used to achieve the expression of the nucleotide sequences coding for an enzyme for use in a method of the invention, in particular an aminotransferase, an amino acid dehydrogenase or a decarboxylase, such as described herein above may be native to the nucleotide sequence coding for the enzyme to be expressed, or may be heterologous to the nucleotide sequence (coding sequence) to which it is operably linked. Preferably, the promoter is homologous, i.e. endogenous to the host cell.

If a heterologous promoter (to the nucleotide sequence encoding for the enzyme of interest) is used, the heterologous promoter is preferably capable of producing a higher steady state level of the transcript comprising the coding sequence (or is capable of producing more transcript molecules, i.e. mRNA molecules, per unit of time) than is the promoter that is native to the coding sequence. Suitable promoters in this context include both constitutive and inducible natural promoters as well as engineered promoters, which are well known to the person skilled in the art.

A “strong constitutive promoter” is one which causes mRNAs to be initiated at high frequency compared to a native host cell. Examples of such strong constitutive promoters in Gram-positive micro-organisms include SP01-26, SP01-15, veg, pyc (pyruvate carboxylase promoter), and amyE.

Examples of inducible promoters in Gram-positive micro-organisms include, the IPTG inducible Pspac promoter, the xylose inducible PxylA promoter.

Examples of constitutive and inducible promoters in Gram-negative microorganisms include, but are not limited to, tac, tet, trp-tet, lpp, lac, lpp-lac, lacIq, T7, T5, T3, gal, trc, ara (P_{BAD}) SP6, λ-P_{R} and λ-P_{L}.

Promoters for (filamentous) fungal cells are known in the art and can be, for example, the glucose-6-phosphate dehydrogenase gpdA promoters, protease promoters such as pepA, pepB, pepC, the glucoamylase glaA promoters, amylase amyA, amyB promoters, the catalase catR or catA promoters, glucose oxidase goxC promoter, beta-galactosidase lacA promoter, alpha-glucosidase aglA promoter, translation elongation factor tefA promoter, xylanase promoters such as xlnA, xlnB, xlnC, xlnD, cellulase promoters such as eglA, egB, cbhA, promoters of transcriptional regulators such as areA, creA, xlnR, pacC, prtT, etc or any other, and can be found among others at the NCBI website (http://www.ncbi.nlm.nih.gov/entrez/)

The invention also relates to a novel heterologous cell which may provide one or more biocatalysts capable of catalysing at least one reaction step in the preparation of AKP, and optionally in the preparation of a further compound from AKP, such as 5-FVA, AAP, 6-ACA, adipic acid, diaminohexane or caprolactam. The invention also relates to a novel vector comprising one or more genes encoding for one or more enzymes capable of catalysing at least one reaction step in the preparation of AKP, and optionally in the preparation of a further compound from AKP, such as 5-FVA, AAP, 6-ACA, adipic acid, diaminohexane or caprolactam. One or more suitable genes may in particular be selected amongst genes encoding an enzyme as mentioned herein above.

The heterologous cell may in particular be a cell as mentioned above when describing the biocatalyst.

In particular, a heterologous cell according to the invention, comprises one or more heterologous nucleic acid sequences (which may be part of one or more vectors) encoding a heterologous enzyme capable of catalysing a reaction step in the preparation of AKP from 2-hydroxyheptanedioic acid.

In a further embodiment, the cell comprises a nucleic acid sequence encoding an enzyme catalysing the preparation of 2-hydroxyheptanedioic acid from heptanedioic acid. Moreover, such a cell may further comprise an enzyme system for catalysing the preparation of heptanedioic acid, from a carbon source.

In a further embodiment, the heterologous cell according to the invention comprises at least one nucleic acid sequence encoding an enzyme for catalysing the conversion of AKP to AAP, 6-ACA, 5-FVA, caprolactam, diaminohexane, or adipic acid. The presence of an nucleic acid sequence encoding such enzyme, is In particular desired in case the cell is intended to be used for preparing a further product from AKP, such as 5-FVA or AAP, which in turn may be further converted to 6-ACA, caprolactam, diaminohexane or adipic acid.

The heterologous cell is preferably free of any enzyme(s) which can degrade or convert AKP, 5-FVA, AAP, 6-ACA, caprolactam, diaminohexane, or adipic acid into any undesired side product. If any such activity e.g. as part of a caprolactam or adipate degradation pathway is identified this activity can be removed, decreased or modified as described herein above.

Inactivation of a gene encoding an undesired activity may be accomplished, by several methods. One approach is a temporary one using an anti-sense molecule or RNAi molecule (e.g. based on Kamath et al. 2003. Nature 421:231-237). Another is using a regulatable promoter system, which can be switched off using external triggers like tetracycline (e.g. based on Park and Morschhauser, 2005, Eukaryot. Cell. 4:1328-1342). Yet another one is to apply a chemical inhibitor or a protein inhibitor or a physical inhibitor (e.g. based on Tour et al. 2003. Nat Biotech 21:1505-1508). A much preferred method is to remove the complete gene(s) or a part thereof, encoding the undesired activity. A further suitable method to modify the genome of a cell in order to prevent it from performing an undesired activity is to inactivate a gene by transposon insertion. To obtain such a mutant one can apply state of the art methods like Single Cross-Over Recombination or Double Homologous Recombination. For this one needs to construct an integrative cloning vector that may integrate at the predetermined target locus in the chromosome of the host cell. In a preferred embodiment of the invention, the integrative cloning vector comprises a DNA fragment, which is homologous to a DNA sequence in a predetermined target locus in the genome of host cell for targeting the integration of the cloning vector to this predetermined locus. In order to promote targeted integration, the cloning vector is preferably linearised prior to transformation of the host cell. Linearisation is preferably performed such that at least one but preferably either end of the cloning vector is flanked by sequences homologous to the target locus. The length of the homologous sequences flanking the target locus is preferably at least 0.1 kb, even preferably at least 0.2 kb, more preferably at least 0.5 kb, even more preferably at least 1 kb, most preferably at least 2 kb. The length that finally is best suitable in an experiment depends on the organism, the sequence and length of the target DNA.

The supply of pimelate, preferably in the cytosolic compartment in the host cell, may be increased by overexpressing homologous and/or heterologous genes encoding enzymes that catalyze the conversion of a precursor molecule to pimelate.

In another aspect, the present invention relates to a process for increasing the production of the AKP or 6-ACA or an intermediate thereof (e.g. pimelate or hydroxypimelate) in a cell, which may be an eukaryotic cell or another cell, capable of producing said compound according to the present invention comprising subjecting a population of eukaryotic cells capable of producing said compound to mutagenesis; and selecting a population of mutant eukaryotic cells for increased production. A small improvement, e.g. of at least 1%, is already interesting. Preferably, the mutagenesis is carried out such that at least 10% of a population of mutant eukaryotic cells shows an increased production as compared to a starting population of eukaryotic cells.

Mutagenesis may be carried out by various methods known in the art, for instance ultraviolet light (UV) mutagenesis, ionizing radiation or incubation with mutagentia. Suitable mutagentia are ethyl methanesulfonate (EMS), diethyl sulfate (DES), methyl methanesulfonate (MMS), dimethyl sulfate (DMS), nitroquinoline oxide (NQO), nitrosoguanidine (NTG), nitrogen mustard (HN2), β-propiolactone, nitrous acid, nitrosoimidazolidone (NIL) and tritiated uridine. A suitable mutagenesis time can be determined based on common general knowledge, depending on e.g. mutagent and organism. The upper limit may be determined by the kill curve. Too large exposure may kill all the cells. Subject to this, the skilled person will be able to determine a suitable upper limit which e.g. may be 3 hours or loss, or one hour or less. After mutagenesis a population of mutant eukaryotic cells for increased production is selected. The mutagenesis of cells and selecting mutant eukaryotic cells for increased production is repeated one or more times.

In a further preferred embodiment, the heterologous cell according to the invention comprises at least one nucleic acid sequence encoding an enzyme represented by SEQ ID NO: 186, SEQ ID NO: 186 or a homologue thereof, which nucleic acid sequence may in particular be selected from the group of SEQ ID NO: 185, SEQ ID NO: 187, SEQ ID NO: 188, SEQ ID NO: 190 and functional analogues thereof. In addition or alternatively, a preferred heterologous cell comprises a enzymes comprising an amino acid sequence as shown Seq ID No: 191, 192, 193, 194, 195, 196, 197, 198, 199, 200, 201, 202, 203, 204, 205, 206, 207, 208 or a homologue of any of these sequences.

In an embodiment, the heterologous cell comprises (a recombinant vector comprising) a nucleic acid sequence encoding an enzyme with alpha-ketopimelic acid aminotransferase activity and/or a nucleic acid sequence encoding an enzyme with alpha-aminopimelic acid decarboxylase activity.

In a preferred embodiment, a heterologous cell according to the invention comprises a nucleic acid sequence encoding an enzyme with AKP decarboxylase activity and/or a nucleic acid sequence encoding an enzyme with 5-FVA aminotransferase activity. In a preferred embodiment, a heterologous cell according to the invention comprises a nucleic acid sequence encoding an enzyme with alpha-aminopimelate 2-dehydrogenase or AKP aminotransferase activity and/or a nucleic acid sequence encoding an enzyme with alpha-aminopimelate decarboxylase activity.

In a preferred embodiment, a heterologous cell according to the invention comprises a nucleic acid sequence encoding an enzyme with 6-aminocaproic acid 6-dehydrogenase activity and optionally a nucleic acid sequence encoding an enzyme with alpha-ketopimelic acid decarboxylase activity.

In a preferred embodiment, a heterologous cell according to the invention comprises a nucleic acid sequence encoding an enzyme with AKP-decarboxylase activity and/or a nucleic acid sequence encoding an enzyme with adipic acid dehydrogenase activity.

The invention is further directed to a nucleic acid comprising a sequence as represented by Sequence ID No: 187, Sequence ID NO: 190 or a non-wild type function analogue thereof.

The invention will now be illustrated by the following examples.

### EXAMPLES

### Part A

### Examples Related to the Preparation of AKP

### General Methods

Molecular and Genetic Techniques

Standard genetic and molecular biology techniques are generally known in the art and have been previously described (Maniatis et al. 1982 “Molecular cloning: a laboratory manual”. Cold Spring Harbor Laboratory, Cold Spring Harbor, N.Y.; Miller 1972 “Experiments in molecular genetics”, Cold Spring Harbor Laboratory, Cold Spring Harbor; Sambrook and Russell 2001 “Molecular cloning: a laboratory manual” (3rd edition), Cold Spring Harbor Laboratory, Cold Spring Harbor Laboratory Press; F. Ausubel et al, eds., “Current protocols in molecular biology”, Green Publishing and Wiley Interscience, New York 1987).

Plasmids and Strains

pMS470 (Balzer, D.; Ziegelin, G.; Pansegrau, W.; Kruft, V.; Lanka, E. Nucleic Acids Research 1992, 20(8), 1851-1858.) and pBBR1MCS (Kovach M E, Phillips R W, Elzer P H, Roop R M 2nd, Peterson K M. Biotechniques. 1994 May; 16(5):800-2. pBBR1MCS: a broad-host-range cloning vector) have been described previously. E. coli strains TOP10 and DH10B (Invitrogen, Carlsbad, Calif., USA) were used for all cloning procedures. E. coli strains BL21 A1 (Invitrogen, Carlsbad, Calif., USA) and BL21 (Novagen (EMD/Merck), Nottingham, UK) were used for protein expression.

pRS414, pRS415 and pRS416 (Sikorski, R. S. and Hieter, P. A system of shuttle vectors and yeast host strains designed for efficient manipulation of DNA in Saccharomyces cerevisiae Genetics 122 (1), 19-27 (1989); Christianson, T. W., Sikorski, R. S., Dante, M., Shero, J. H. and Hieter, P. Multifunctional yeast high-copy-number shuttle vectors. Gene 110 (1), 119-122 (1992)) were used for expression in S. cerevisiae. S. cerevisiae strains CEN.PK 113-6B (ura3, trp1, leu2, MATa), CEN.PK 113-5D (ura3, MATa), CEN.PK 102-3A (ura3, leu2, MATa) and CEN.PK 113-9D (ura3, trp1, MATa) were used for protein expression.

Media

2×TY medium (16 g/l tryptopeptone, 10 g/l yeast extract, 5 g/l NaCl) was used for growth of E. coli. Antibiotics (100 μg/ml ampicillin, 50-100 μg/ml neomycin) were supplemented to maintain plasmids in E. coli. For induction of gene expression in E. coli arabinose (for BL21-AI derivatives) and IPTG (for pMS470, pBBR1MCS derivatives) were used at 0.02% (arabinose) and 0.2 mM (IPTG) final concentrations.

Verduyn medium with 4% galactose was used for growth of S. cerevisiae.

Identification of Plasmids

Plasmids carrying the different genes were identified by genetic, biochemical, and/or phenotypic means generally known in the art, such as resistance of transformants to antibiotics, PCR diagnostic analysis of transformant or purification of plasmid DNA, restriction analysis of the purified plasmid DNA or DNA sequence analysis. Integrity of all new constructs described was confirmed by restriction digest and, if PCR steps were involved, additionally by sequencing.

UPLC-MS/MS Analysis Method for the Determination of α-Keto Acids

A Waters HSS T3 column 1.8 μm, 100 mm*2.1 mm was used for the separation of a-keto acids with gradient elution as depicted in Table 1. Eluens A consists of LC/MS grade water, containing 0.1% formic acid, and eluens B consists of acetonitrile, containing 0.1% formic acid. The flow-rate was 0.25 ml/min and the column was thermostated at a temperature of 40° C.

TABLE 1
gradient elution program used for the separation
of α-keto acids, 6-ACA, 5-FVA and homo₍ₙ₎citrate

| Time (min) | 0 | 5.0 | 5.5 | 10 | 10.5 | 15 |
|---|---|---|---|---|---|---|
| % A | 100 | 85 | 20 | 20 | 100 | 100 |
| % B | 0 | 15 | 80 | 80 | 0 | 0 |

A Waters micromass Quattro micro API was used in electrospray either positive or negative ionization mode, depending on the compounds to be analyzed, using multiple reaction monitoring (MRM). The ion source temperature was kept at 130° C., whereas the desolvation temperature is 350° C., at a flow-rate of 500 L/hr.

For AKP the deprotonated molecule was fragmented with 10-14 eV, resulting in specific fragments from losses of e.g. H₂O, CO and CO₂.

To determine concentrations a standard curve of synthetically prepared compounds was run to calculate a response factor for the respective ions. This was used to calculate the concentrations in unknown samples.

### Synthesis of 2-hydroxyheptanedioic acid

This method illustrates how 2-hydroxyheptanedioic acid (HPDA) was made from AKP, which HPDA was synthesized for use in testing Purposes)

2-Hydroxyheptanedioic acid for use as a substrate for the biocatalytic production of AKP was synthesised by hydrogenation of AKP (provided by Syncom). AKP (2.2 g, 12.6 mmol) was dissolved in methanol (50 mL) to this 30 mg of Pd on charcoal was added (Pd/C, 5%) and placed in an autoclave under a hydrogen pressure of 30 bar at 50° C. for 48 hours. The reaction mixture was allowed reach room temperature and subsequently filtered over Celite® and concentrated in vacuo to yield the title compound as oil (2.2 g, 99%).

The product was characterised by ¹H-NMR and ¹³C-NMR

¹H-NMR (300 MHz, DMSO): δ 4.02-3.98 and 3.92-3.89 (dd, ³J=7.6 Hz, ³J=4.8 Hz, 1H), 2.28 and 2.18 (t, ³J=7.2 Hz, 2H), 1.66-1.28 (m, 6H) ¹³C-NMR (75 MHz, DMSO): δ 174.9, 173.6, 70.0, 51.6, 34.0, 33.6, 24.6

### Example 1

### Preparation of pBAD-DEST Top10 Cell with Heterologous Hydroxyacid Oxidase

HAOX5B (SEQ ID NO: 187) and LAOX8C (SEQ ID NO: 190) were obtained by DNA synthesis. attB sites were added to all genes upstream of the ribosomal binding site and start codon and downstream of the stop codon to facilitate cloning using the Gateway technology (Invitrogen, Carlsbad, Calif., USA). The gene constructs were cloned into pBAD/Myc-His-DEST expression vectors using the Gateway technology (Invitrogen) via the introduced attB sites and pDONR201 (Invitrogen) as entry vector as described in the manufacturer's protocols (www.invitrogen.com). This way the expression vectors pBAD-Vfl_AT and pBAD-Bwe_AT were obtained, respectively. The corresponding expression strains were obtained by transformation of chemically competent E. coli TOP10 (Invitrogen) with the respective pBAD-expression vectors.

### Example 2

### Growth of E. coli for Protein Expression

Small scale growth of the cells prepared in Example 1 was carried out in 96-deep-well plates with 940 μl media containing 0.02% (w/v) L-arabinose. Inoculation was performed by transferring cells from frozen stock cultures with a 96-well stamp (Kühner, Birsfelden, Switzerland). Plates were incubated on an orbital shaker (300 rpm, 5 cm amplitude) at 25° C. for 48 h. Typically an OD₆₂₀ₙₘ of 2-4 was reached.

### Example 3

### Preparation of Cell Lysates

The lysis buffer contained the following ingredients:

TABLE 2
lysis buffer contents

| | | |
|---|---|---|
| 1M MOPS pH 7.5 | 5 | ml |
| DNAse I grade II (Roche) | 10 | mg |
| Lysozyme | 200 | mg |
| MgSO₄•7H₂O | 123.2 | mg |
| dithiothreitol (DTT) | 154.2 | mg |

| | |
|---|---|
| H₂O (MilliQ) | Balance to 100 ml |

The solution was freshly prepared directly before use.

Cells from small scales growth (see Example 2) were harvested by centrifugation and the supernatant was discarded. The cell pellets formed during centrifugation were frozen at −20° C. for at least 16 h and then thawed on ice. 500 μl of freshly prepared lysis buffer were added to each well and cells were resuspended by vigorously vortexing the plate for 2-5 min. To achieve lysis, the plate was incubated at room temperature for 30 min. To remove cell debris, the plate was centrifuged at 4° C. and 6000 g for 20 min. The supernatant (comprising hydroxyacid oxidase, either HAOX 5B or LAOX 8C) was transferred to a fresh plate and kept on ice until further use.

### Example 4

### Enzymatic Preparation of AKP

2-Hydroxyheptanedioic acid (final concentration 50 mM, >95% purity, obtained as described above) was contacted with hydroxyacid oxidase (either HAOX 5B or LAOX 8C), obtained as described in Example 3 in a buffer solution containing the following.
4-aminoantipyrine (1 mM)
3,5-dichloro-2-hydroxybenzenesulfonic acid (DCHBS) (10 mM)
50 mM potassium phosphate buffer, pH 7.5
Horseradish peroxidase (200 μml)

Reactions were incubated for 20 h at 37 C. Samples were frozen and prior to analysis heated to 95 C for 2 min to precipitate protein. After centrifugation the supernatant was analyzed by UPLC-MS.AKP concentration in the sample from the test with HAOX 5B was 59 mg/l, and AKP concentration in the sample from the test with LAOX 8C was 58 mg/l.

### Example 5

### Enzymatic Preparation of 5-FVA from AKP

5-FVA can be prepared from AKP as described in the Examples of WO 2009/113855:

A reaction mixture was prepared comprising 50 mM AKP, 5 mM magnesium chloride, 100 μM pyridoxal 5′-phosphate (for LysA) or 1 mM thiamine diphosphate (for all other enzymes) in 100 mM potassium phosphate buffer, pH 6.5. 4 ml of the reaction mixture were dispensed into a reaction vessel. To start the reaction, 1 ml of the cell free extracts obtained by sonification were added, to each of the wells. In case of the commercial oxaloacetate decarboxylase (Sigma-Aldrich product number 04878), 50 U were used. Reaction mixtures were incubated with a magnetic stirrer at 37° C. for 48 h. Furthermore, a chemical blank mixture (without cell free extract) and a biological blank (E. coli TOP10 with pBAD/Myc-His C) were incubated under the same conditions. Samples from different time points during the reaction were analysed by HPLC-MS. The results are summarised in the following table.

TABLE 3
5-FVA formation from AKP in the presence of decarboxylases (see
Examples of WO 2009/113855 for preparation of biocatalyst)

| | 5-FVA concentration [mg/kg] | | |
|---|---|---|---|
| Biocatalyst | 3 h | 18 h | 48 h |
| E. coli TOP10/pBAD-LysA | 150 | 590 | 720 |
| E. coli TOP10/pBAD-Pdc | 1600 | 1700 | 1300 |
| E. coli TOP10/pBAD-Pdcl472A | 2000 | 2000 | 1600 |
| E. coli TOP10/pBAD-KdcA | 3300 | 2300 | 2200 |
| E. coli TOP10/pBAD-KivD | 820 | 1400 | 1500 |
| Oxaloacetate decarboxylase | n.d. | 6 | 10 |
| E. coli TOP10 with pBAD/ | n.d. | n.d. | n.d. |
| Myc-His C (biological blank) | | | |
| None (chemical blank) | n.d. | n.d. | n.d. |

n.d.: not detectable

It is shown that 5-FVA is formed from AKP in the presence of a decarboxylase.

### Example 6

### Enzymatic Preparation of 6-ACA from AKP

6-ACA can be prepared from AKP as described in the Examples of WO 2009/113855:

A reaction mixture was prepared comprising 50 mM AKP, 5 mM magnesium chloride, 100 μM pyridoxal 5′-phosphate (for LysA) or 1 mM thiamine diphosphate (for all other tested biocatalysts) in 100 mM potassium phosphate buffer, pH 6.5. 4 ml of the reaction mixture were dispensed into a reaction vessel. To start the reaction, 1 ml of the cell free extracts were added, to each of the wells. Reaction mixtures were incubated with a magnetic stirrer at 37° C. for 48 h. Furthermore, a chemical blank mixture (without cell free extract) and a biological blank (E. coli TOP10 with pBAD/Myc-His C) were incubated under the same conditions. Samples from different time points during the reaction were analysed by HPLC-MS. The results are summarised in the following table.

TABLE 4
6-ACA formation from AKP in the presence of decarboxylases (see
Examples of WO 2009/113855 for preparation of biocatalyst)

| | 6-ACA concentration | | |
|---|---|---|---|
| | [mg/kg] | | |
| Biocatalyst | 3 h | 18 h | 48 h |
| E. coli TOP10/pBAD-LysA | n.a. | 0.01 | 0 |
| E. coli TOP10/pBAD-Pdc | 0.1 | 0.3 | n.a. |
| E. coli TOP10/pBAD-Pdcl472A | 0.03 | 0.1 | 0.2 |
| E. coli TOP10/pBAD-KdcA | 0.04 | 0.1 | 0.3 |
| E. coli TOP10/pBAD-KivD | n.a. | 0.3 | 0.6 |
| E. coli TOP10 with pBAD/ | n.d. | n.d. | n.d. |
| Myc-His C (biological blank) | | | |
| None (chemical blank) | n.d. | n.d. | n.d. |

n.a. = not analysed
n.d. = not detectable

It is shown that 6-ACA is formed from AKP in the presence of a decarboxylase. It is contemplated that the E. coli contained natural 5-FVA aminotransferase activity.

### Example 7

### Enzymatic Preparation of 6-ACA from AKP in Presence of Recombinant Decarboxylase and Recombinant Aminotransferase

A reaction mixture was prepared comprising 50 mM AKP, 5 mM magnesium chloride, 100 μM pyridoxal 5′-phosphate, 1 mM thiamine diphosphate and 50 mM racemic α-methylbenzylamine in 100 mM potassium phosphate buffer, pH 6.5. 1.6 ml of the reaction mixture were dispensed into a reaction vessel. To start the reaction, 0.2 ml of the decarboxylase containing cell free extract and 0.2 ml of the aminotransferase containing cell free extract were added, to each of the reaction vessels. Reaction mixtures were incubated with a magnetic stirrer at 37° C. for 48 h. Furthermore, a chemical blank mixture (without cell free extract) and a biological blank (E. coli TOP10 with pBAD/Myc-His C) were incubated under the same conditions. Samples from different time points during the reaction were analysed by HPLC-MS. The results are summarised in the following table.

TABLE 5
6-ACA formation from AKP in the presence of a recombinant
decarboxylase and a recombinant aminotransferase (see Examples
of WO 2009/113855 for preparation of biocatalyst)

| | 6-ACA concentration [mg/kg] after 48 hours | | |
|---|---|---|---|
| | AT | | |
| | E. coli TOP10/ | E. coli TOP10/ | E. coli TOP10/pBAD- |
| DC | pBAD-Vfl-AT | pBAD-Bwe-AT | PAE_gi9946143_AT |
| E. coli | 183.4 | 248.9 | 117.9 |
| TOP10/ | | | |
| pBAD-Pdc | | | |
| E. coli | 458.5 | 471.6 | 170.3 |
| TOP10/ | | | |
| pBAD- | | | |
| Pdcl472A | | | |
| E. coli | 497.8 | 497.8 | 275.1 |
| TOP10/ | | | |
| pBAD-KdcA | | | |
| E. coli | 510.9 | 510.9 | 314.4 |
| TOP10/ | | | |
| pBAD-KivD | | | |

AT = aminotransferase
DC = decarboxylase

In the chemical blank and in the biological blank no 6-ACA was detectable.

Further, the results show that compared to the example wherein a host-cell with only recombinant decarboxylase (and no recombinant aminotransferase) the conversion to 6-ACA was improved.

### Example 8

### Enzymatic Reactions for Conversion of AKP to 6-ACA in Presence of Decarboxylase and Aminotransferase Co-Expressed in S. cerevisiae

A reaction mixture was prepared comprising 50 mM AKP, 5 mM magnesium chloride, 100 μM pyridoxal 5′-phosphate, 1 mM thiamine diphosphate and 50 mM racemic a-methylbenzylamine in 100 mM potassium phosphate buffer, pH 6.5. 1.6 ml of the reaction mixture were dispensed into a reaction vessel. To start the reaction, 0.4 ml of the cell free extract from S. cerevisiae containing decarboxylase and aminotransferase were added, to each of the reaction vessels. Reaction mixtures were incubated with a magnetic stirrer at 37° C. Furthermore, a chemical blank mixture (without cell free extract) and a biological blank (S. cerevisiae) were incubated under the same conditions. Samples, taken after 19 hours of incubation, were analysed by HPLC-MS. The results are summarised in the following table.

TABLE 6
6-ACA formation from AKP using a micro-organism as a biocatalyst
(see Examples of WO 2009/113855 for preparation of biocatalyst)

| | 6-ACA concentration |
|---|---|
| Biocatalyst | [mg/kg] |
| S. cerevisiae pAKP-85 | 63 |
| S. cerevisiae pAKP-86 | 226 |
| S. cerevisiae pAKP-87 | 1072 |
| S. cerevisiae pAKP-88 | 4783 |
| S. cerevisiae | 3.9 |
| (biological blank) | |
| None (chemical blank) | 1.3 |

### Example 9

### Enzymatic Reactions for Conversion of Alpha-Ketopimelic Acid to Alpha-Aminopimelic Acid

A reaction mixture was prepared comprising 10 mM alpha-ketopimelic acid, 20 mM L-alanine, and 50 μM pyridoxal 5′-phosphate in 50 mM potassium phosphate buffer, pH 7.0. 800 μl of the reaction mixture were dispensed into each well of the well plates. To start the reaction, 200 μl of the cell lysates were added, to each of the wells. Reaction mixtures were incubated on a shaker at 37° C. for 24 h. Furthermore, a chemical blank mixture (without cell free extract) and a biological blank (E. coli TOP10 with pBAD/Myc-His C) were incubated under the same conditions. Samples were analysed by HPLC-MS. The results are summarised in the following table.

TABLE 7
AAP formation from AKP in the presence of aminotransferases (see
Examples of WO 2009/113855 for preparation of biocatalyst)

| | AAP concentration [mg/kg] |
|---|---|
| Biocatalyst | (after 24 hrs) |
| E. coli TOP10/pBAD-Vfl_AT | 3.7 |
| E. coli TOP10/pBAD-Psy_AT | 15.8 |
| E. coli TOP10/pBAD-Bsu_gi16078032_AT | 11.2 |
| E. coli TOP10/pBAD-Rsp_AT | 9.8 |
| E. coli TOP10/pBAD-Bsu_gi16080075_AT | 4.6 |
| E. coli TOP10/pBAD-Lpn_AT | 5.4 |
| E. coli TOP10/pBAD-Neu_AT | 7.7 |
| E. coli TOP10/pBAD-Ngo_AT | 5.1 |
| E. coli TOP10/pBAD-Pae_gi9951299_AT | 5.6 |
| E. coli TOP10/pBAD-Rpa_AT | 5.4 |
| E. coli TOP10 with pBAD/Myc-His C | 1.4 |
| (biological blank) | |
| None (chemical blank) | 0 |

It is shown that the formation of AAP from AKP is catalysed by the biocatalyst.

### Example 10

### Chemical Conversion of AAP to Caprolactam

To a suspension of 1.5 grams of D,L-2-aminopimelic acid in 21 ml cyclohexanone, 0.5 ml of cyclohexenone was added. The mixture was heated on an oil bath for 20 h at reflux (approximately 160° C.). After cooling to room temperature the reaction mixture was decanted and the clear solution was evaporated under reduced pressure. The remaining 2 grams of brownish oil were analyzed by ¹H-NMR and HPLC and contained 0.8 wt % caprolactam and 6 wt % of cyclic oligomers of caprolactam.

SEQUENCES:

SEQ ID NO: 1
DNA - Vibrio fluvialis
atg aac aaa ccg caa agc tgg gaa gcc cgg gcc gag acc tat tcg ctc
Met Asn Lys Pro Gln Ser Trp Glu Ala Arg Ala Glu Thr Tyr Ser Leu
tat ggt ttc acc gac atg cct tcg ctg cat cag cgc ggc acg gtc gtc
Tyr Gly Phe Thr Asp Met Pro Ser Leu His Gln Arg Gly Thr Val Val
gtg acc cat ggc gag gga ccc tat atc gtc gat gtg aat ggc cgg cgt
Val Thr His Gly Glu Gly Pro Tyr Ile Val Asp Val Asn Gly Arg Arg
tat ctg gac gcc aac tcg ggc ctg tgg aac atg gtc gcg ggc ttt gac
Tyr Leu Asp Ala Asn Ser Gly Leu Trp Asn Met Val Ala Gly Phe Asp
cac aag ggg ctg atc gac gcc gcc aag gcc caa tac gag cgt ttt ccc
His Lys Gly Leu Ile Asp Ala Ala Lys Ala Gln Tyr Glu Arg Phe Pro
ggt tat cac gcc ttt ttc ggc cgc atg tcc gat cag acg gta atg ctg
Gly Tyr His Ala Phe Phe Gly Arg Met Ser Asp Gln Thr Val Met Leu
tcg gaa aag ctg gtc gag gtg tcg ccc ttt gat tcg ggc cgg gtg ttc
Ser Glu Lys Leu Val Glu Val Ser Pro Phe Asp Ser Gly Arg Val Phe
tat aca aac tcg ggg tcc gag gcg aat gac acc atg gtc aag atg cta
Tyr Thr Asn Ser Gly Ser Glu Ala Asn Asp Thr Met Val Lys Met Leu
tgg ttc ctg cat gca gcc gag ggc aaa ccg caa aag cgc aag atc ctg
Trp Phe Leu His Ala Ala Glu Gly Lys Pro Gln Lys Arg Lys Ile Leu
acc cgc tgg aac gcc tat cac ggc gtg acc gcc gtt tcg gcc agc atg
Thr Arg Trp Asn Ala Tyr His Gly Val Thr Ala Val Ser Ala Ser Met
acc ggc aag ccc tat aat tcg gtc ttt ggc ctg ccg ctg ccg ggc ttt
Thr Gly Lys Pro Tyr Asn Ser Val Phe Gly Leu Pro Leu Pro Gly Phe
gtg cat ctg acc tgc ccg cat tac tgg cgc tat ggc gaa gag ggc gaa
Val His Leu Thr Cys Pro His Tyr Trp Arg Tyr Gly Glu Glu Gly Glu
acc gaa gag cag ttc gtc gcc cgc ctc gcc cgc gag ctg gag gaa acg
Thr Glu Glu Gln Phe Val Ala Arg Leu Ala Arg Glu Leu Glu Glu Thr
atc cag cgc gag ggc gcc gac acc atc gcc ggt ttc ttt gcc gaa ccg
Ile Gln Arg Glu Gly Ala Asp Thr Ile Ala Gly Phe Phe Ala Glu Pro
gtg atg ggc gcg ggc ggc gtg att ccc ccg gcc aag ggc tat ttc cag
Val Met Gly Ala Gly Gly Val Ile Pro Pro Ala Lys Gly Tyr Phe Gln
gcg atc ctg cca atc ctg cgc aaa tat gac atc ccg gtc atc tcg gac
Ala Ile Leu Pro Ile Leu Arg Lys Tyr Asp Ile Pro Val Ile Ser Asp
gag gtg atc tgc ggt ttc gga cgc acc ggt aac acc tgg ggc tgc gtg
Glu Val Ile Cys Gly Phe Gly Arg Thr Gly Asn Thr Trp Gly Cys Val
acc tat gac ttt aca ccc gat gca atc atc tcg tcc aag aat ctt aca
Thr Tyr Asp Phe Thr Pro Asp Ala Ile Ile Ser Ser Lys Asn Leu Thr
gcg ggc ttt ttc ccc atg ggg gcg gtg atc ctt ggc ccg gaa ctt tcc
Ala Gly Phe Phe Pro Met Gly Ala Val Ile Leu Gly Pro Glu Leu Ser
aaa cgg ctg gaa acc gca atc gag gcg atc gag gaa ttc ccc cat ggc
Lys Arg Leu Glu Thr Ala Ile Glu Ala Ile Glu Glu Phe Pro His Gly
ttt acc gcc tcg ggc cat ccg gtc ggc tgt gct att gcg ctg aaa gca
Phe Thr Ala Ser Gly His Pro Val Gly Cys Ala Ile Ala Leu Lys Ala
atc gac gtg gtg atg aat gaa ggg ctg gct gag aac gtc cgc cgc ctt
Ile Asp Val Val Met Asn Glu Gly Leu Ala Glu Asn Val Arg Arg Leu
gcc ccc cgt ttc gag gaa agg ctg aaa cat atc gcc gag cgc ccg aac
Ala Pro Arg Phe Glu Glu Arg Leu Lys His Ile Ala Glu Arg Pro Asn
atc ggt gaa tat cgc ggc atc ggc ttc atg tgg gcg ctg gag gct gtc
Ile Gly Glu Tyr Arg Gly Ile Gly Phe Met Trp Ala Leu Glu Ala Val
aag gac aag gca agc aag acg ccg ttc gac ggc aac ctg tcg gtc agc
Lys Asp Lys Ala Ser Lys Thr Pro Phe Asp Gly Asn Leu Ser Val Ser
gag cgt atc gcc aat acc tgc acc gat ctg ggg ctg att tgc cgg ccg
Glu Arg Ile Ala Asn Thr Cys Thr Asp Leu Gly Leu Ile Cys Arg Pro
ctt ggt cag tcc gtc gtc ctt tgt ccg ccc ttt atc ctg acc gag gcg
Leu Gly Gln Ser Val Val Leu Cys Pro Pro Phe Ile Leu Thr Glu Ala
cag atg gat gag atg ttc gat aaa ctc gaa aaa gcc ctt gat aag gtc
Gln Met Asp Glu Met Phe Asp Lys Leu Glu Lys Ala Leu Asp Lys Val
ttt gcc gag gtt gcc tga
Phe Ala Glu Val Ala
SEQ ID NO: 2
PRT - Vibrio fluvialis
Met Asn Lys Pro Gln Ser Trp Glu Ala Arg Ala Glu Thr Tyr Ser Leu
Tyr Gly Phe Thr Asp Met Pro Ser Leu His Gln Arg Gly Thr Val Val
Val Thr His Gly Glu Gly Pro Tyr Ile Val Asp Val Asn Gly Arg Arg
Tyr Leu Asp Ala Asn Ser Gly Leu Trp Asn Met Val Ala Gly Phe Asp
His Lys Gly Leu Ile Asp Ala Ala Lys Ala Gln Tyr Glu Arg Phe Pro
Gly Tyr His Ala Phe Phe Gly Arg Met Ser Asp Gln Thr Val Met Leu
Ser Glu Lys Leu Val Glu Val Ser Pro Phe Asp Ser Gly Arg Val Phe
Tyr Thr Asn Ser Gly Ser Glu Ala Asn Asp Thr Met Val Lys Met Leu
Trp Phe Leu His Ala Ala Glu Gly Lys Pro Gln Lys Arg Lys Ile Leu
Thr Arg Trp Asn Ala Tyr His Gly Val Thr Ala Val Ser Ala Ser Met
Thr Gly Lys Pro Tyr Asn Ser Val Phe Gly Leu Pro Leu Pro Gly Phe
Val His Leu Thr Cys Pro His Tyr Trp Arg Tyr Gly Glu Glu Gly Glu
Thr Glu Glu Gln Phe Val Ala Arg Leu Ala Arg Glu Leu Glu Glu Thr
Ile Gln Arg Glu Gly Ala Asp Thr Ile Ala Gly Phe Phe Ala Glu Pro
Val Met Gly Ala Gly Gly Val Ile Pro Pro Ala Lys Gly Tyr Phe Gln
Ala Ile Leu Pro Ile Leu Arg Lys Tyr Asp Ile Pro Val Ile Ser Asp
Glu Val Ile Cys Gly Phe Gly Arg Thr Gly Asn Thr Trp Gly Cys Val
Thr Tyr Asp Phe Thr Pro Asp Ala Ile Ile Ser Ser Lys Asn Leu Thr
Ala Gly Phe Phe Pro Met Gly Ala Val Ile Leu Gly Pro Glu Leu Ser
Lys Arg Leu Glu Thr Ala Ile Glu Ala Ile Glu Glu Phe Pro His Gly
Phe Thr Ala Ser Gly His Pro Val Gly Cys Ala Ile Ala Leu Lys Ala
Ile Asp Val Val Met Asn Glu Gly Leu Ala Glu Asn Val Arg Arg Leu
Ala Pro Arg Phe Glu Glu Arg Leu Lys His Ile Ala Glu Arg Pro Asn
Ile Gly Glu Tyr Arg Gly Ile Gly Phe Met Trp Ala Leu Glu Ala Val
Lys Asp Lys Ala Ser Lys Thr Pro Phe Asp Gly Asn Leu Ser Val Ser
Glu Arg Ile Ala Asn Thr Cys Thr Asp Leu Gly Leu Ile Cys Arg Pro
Leu Gly Gln Ser Val Val Leu Cys Pro Pro Phe Ile Leu Thr Glu Ala
Gln Met Asp Glu Met Phe Asp Lys Leu Glu Lys Ala Leu Asp Lys Val
Phe Ala Glu Val Ala
SEQ ID NO: 3
DNA - Artificial
Vibrio fluvialis JS17 omega-aminotransferase codon optimised gene
atgaataaac cacagtcttg ggaagctcgt gctgaaacct atagcctgta cggctttacc
gatatgccgt ctctgcacca gcgtggtact gtagtggtaa cgcacggtga gggcccgtac
atcgtggacg ttaatggccg ccgttacctg gatgcaaaca gcggcctgtg gaacatggtt
gcgggcttcg accacaaagg cctgatcgat gccgcaaaag cgcagtacga acgcttcccg
ggttatcacg cgttctttgg ccgtatgagc gaccagactg tgatgctgag cgaaaaactg
gttgaagtgt ccccgttcga tagcggtcgt gtcttttaca ctaactctgg cagcgaggct
aacgatacca tggttaagat gctgtggttc ctgcacgcag cggaaggcaa acctcagaaa
cgtaaaattc tgacccgttg gaacgcttat cacggtgtga ctgctgtttc cgcatctatg
accggtaaac cgtataacag cgtgttcggt ctgccgctgc ctggcttcgt gcatctgacc
tgcccgcact actggcgtta tggtgaggaa ggcgaaactg aggaacagtt cgtggcgcgt
ctggctcgtg aactggaaga aaccattcaa cgcgaaggtg cagatactat cgcgggcttc
tttgcggagc ctgttatggg tgccggcggt gtgattccgc cggcgaaggg ctatttccag
gcaatcctgc cgatcctgcg caagtacgac attccggtta tttctgacga agtgatctgc
ggcttcggcc gcaccggtaa cacctggggc tgcgtgacgt atgacttcac tccggacgca
atcattagct ctaaaaacct gactgcgggt ttcttcccta tgggcgccgt aatcctgggc
ccagaactgt ctaagcgcct ggaaaccgcc atcgaggcaa tcgaagagtt cccgcacggt
ttcactgcta gcggccatcc ggtaggctgc gcaatcgcgc tgaaggcgat cgatgttgtc
atgaacgagg gcctggcgga aaacgtgcgc cgcctggcgc cgcgttttga agaacgtctg
aaacacattg ctgagcgccc gaacattggc gaatatcgcg gcatcggttt catgtgggcc
ctggaagcag ttaaagataa agctagcaag accccgttcg acggcaacct gtccgtgagc
gaacgtatcg ctaatacctg tacggacctg ggtctgatct gccgtccgct gggtcagtcc
gtagttctgt gcccaccatt tatcctgacc gaagcgcaga tggatgaaat gttcgataaa
ctggagaaag ctctggataa agtgttcgct gaagtcgcgt aa
SEQ ID NO: 4
PRT - Methanocaldococcus jannashii DSM2661
Met Thr Lys Val Leu Val Met Phe Met Asp Phe Leu Phe Glu Asn Ser
Trp Lys Ala Val Cys Pro Tyr Asn Pro Lys Leu Asp Leu Lys Asp Ile
Tyr Ile Tyr Asp Thr Thr Leu Arg Asp Gly Glu Gln Thr Pro Gly Val
Cys Phe Thr Lys Glu Gln Lys Leu Glu Ile Ala Arg Lys Leu Asp Glu
Leu Gly Leu Lys Gln Ile Glu Ala Gly Phe Pro Ile Val Ser Glu Arg
Glu Ala Asp Ile Val Lys Thr Ile Ala Asn Glu Gly Leu Asn Ala Asp
Ile Leu Ala Leu Cys Arg Ala Leu Lys Lys Asp Ile Asp Lys Ala Ile
Glu Cys Asp Val Asp Gly Ile Ile Thr Phe Ile Ala Thr Ser Pro Leu
His Leu Lys Tyr Lys Phe Asn Asn Lys Ser Leu Asp Glu Ile Leu Glu
Met Gly Val Glu Ala Val Glu Tyr Ala Lys Glu His Gly Leu Phe Val
Ala Phe Ser Ala Glu Asp Ala Thr Arg Thr Pro Ile Glu Asp Leu Ile
Lys Val His Lys Ala Ala Glu Glu Ala Gly Ala Asp Arg Val His Ile
Ala Asp Thr Thr Gly Cys Ala Thr Pro Gln Ser Met Glu Phe Ile Cys
Lys Thr Leu Lys Glu Asn Leu Lys Lys Ala His Ile Gly Val His Cys
His Asn Asp Phe Gly Phe Ala Val Ile Asn Ser Ile Tyr Gly Leu Ile
Gly Gly Ala Lys Ala Val Ser Thr Thr Val Asn Gly Ile Gly Glu Arg
Ala Gly Asn Ala Ala Leu Glu Glu Leu Ile Met Ala Leu Thr Val Leu
Tyr Asp Val Asp Leu Gly Leu Asn Leu Glu Val Leu Pro Glu Leu Cys
Arg Met Val Glu Glu Tyr Ser Gly Ile Lys Met Pro Lys Asn Lys Pro
Ile Val Gly Glu Leu Val Phe Ala His Glu Ser Gly Ile His Val Asp
Ala Val Ile Glu Asn Pro Leu Thr Tyr Glu Pro Phe Leu Pro Glu Lys
Ile Gly Leu Lys Arg Asn Ile Leu Leu Gly Lys His Ser Gly Cys Arg
Ala Val Ala Tyr Lys Leu Lys Leu Met Gly Ile Asp Tyr Asp Arg Glu
Met Leu Cys Glu Ile Val Lys Lys Val Lys Glu Ile Arg Glu Glu Gly
Lys Phe Ile Thr Asp Glu Val Phe Lys Glu Ile Val Glu Glu Val Leu
Arg Lys Arg Asn Lys Asn
SEQ ID NO: 5
PRT - Methanothermobacter thermoautotropicum DH
Met Arg Tyr Phe Val Ser Pro Phe Asn Lys Glu Ala Glu Leu Lys Phe
Pro Asp Arg Ile Thr Ile Tyr Asp Thr Thr Leu Arg Asp Gly Glu Gln
Thr Pro Gly Val Cys Leu Gly Thr Glu Glu Lys Leu Glu Ile Ala Arg
Lys Leu Asp Glu Leu Gly Ile His Gln Ile Glu Ser Gly Phe Pro Val
Val Ser Glu Gln Glu Arg Val Ser Val Lys Ser Ile Ala Asn Glu Gly
Leu Asn Ala Glu Ile Leu Ala Leu Cys Arg Thr Lys Lys Asp Asp Ile
Asp Ala Ala Ile Asp Cys Asp Val Asp Gly Val Ile Thr Phe Met Ala
Thr Ser Asp Leu His Leu Lys His Lys Leu Lys Leu Thr Arg Glu Glu
Ala Leu Asn Val Cys Met Asn Ser Ile Glu Tyr Ala Lys Asp His Gly
Leu Phe Leu Ala Phe Ser Ala Glu Asp Ala Thr Arg Thr Asp Leu Asp
Phe Leu Lys Gln Ile Tyr Arg Lys Ala Glu Asn Tyr Gly Ala Asp Arg
Val His Ile Ala Asp Thr Val Gly Ala Ile Ser Pro Gln Gly Met Asp
Tyr Leu Val Arg Glu Leu Arg Arg Asp Ile Lys Val Asp Ile Ala Leu
His Cys His Asn Asp Phe Gly Met Ala Leu Ser Asn Ser Ile Ala Gly
Leu Leu Ala Gly Gly Thr Ala Val Ser Thr Thr Val Asn Gly Ile Gly
Glu Arg Ala Gly Asn Thr Ser Leu Glu Glu Leu Ile Met Ala Leu Arg
Ile Ile Tyr Glu Val Asp Leu Gly Phe Asn Ile Gly Val Leu Tyr Glu
Leu Ser Arg Leu Val Glu Lys His Thr Arg Met Lys Val Pro Glu Asn
Lys Pro Ile Val Gly Arg Asn Val Phe Arg His Glu Ser Gly Ile His
Val Asp Ala Val Ile Glu Glu Pro Leu Thr Tyr Glu Pro Phe Leu Pro
Glu Met Ile Gly His Gln Arg Lys Ile Val Leu Gly Lys His Ser Gly
Cys Arg Ala Val Lys Ala Lys Leu Glu Glu Tyr Gly Ile Asp Val Thr
Arg Asp Glu Leu Cys Arg Ile Val Glu Glu Val Lys Lys Asn Arg Glu
Lys Gly Lys Tyr Ile Asn Asp Glu Leu Phe Tyr Arg Ile Val Lys Ser
Val Arg Gly Pro Val Asp Phe
SEQ ID NO: 6
PRT - Methanococcus maripaludis S2
Met Asp Trp Lys Ala Val Ser Pro Tyr Asn Pro Lys Leu Asp Leu Lys
Asp Cys Tyr Leu Tyr Asp Thr Thr Leu Arg Asp Gly Glu Gln Thr Pro
Gly Val Cys Phe Ala Gly Asp Gln Lys Leu Glu Ile Ala Lys Lys Leu
Asp Glu Leu Lys Ile Lys Gln Ile Glu Ala Gly Phe Pro Ile Val Ser
Glu Asn Glu Arg Lys Ala Ile Lys Ser Ile Thr Gly Glu Gly Leu Asn
Ala Gln Ile Leu Ala Leu Ser Arg Val Leu Lys Glu Asp Ile Asp Lys
Ala Ile Glu Cys Asp Val Asp Gly Ile Ile Thr Phe Ile Ala Thr Ser
Pro Met His Leu Lys Tyr Lys Leu His Lys Asn Leu Asp Glu Val Glu
Glu Met Gly Met Lys Ala Val Glu Tyr Ala Lys Asp His Gly Leu Phe
Val Ala Phe Ser Ala Glu Asp Ala Thr Arg Thr Pro Leu Glu Asp Ile
Ile Arg Ile His Lys Asn Ala Glu Glu His Gly Ala Asp Arg Val His
Ile Ala Asp Thr Leu Gly Cys Ala Thr Pro Gln Ala Met Tyr His Ile
Cys Ser Glu Leu Ser Lys His Leu Lys Lys Ala His Ile Gly Val His
Cys His Asn Asp Phe Gly Phe Ala Val Ile Asn Ser Ile Tyr Gly Leu
Ile Gly Gly Ala Lys Ala Val Ser Thr Thr Val Asn Gly Ile Gly Glu
Arg Ala Gly Asn Ala Ala Ile Glu Glu Ile Ala Met Ala Leu Lys Val
Leu Tyr Asp His Asp Met Gly Leu Asn Thr Glu Ile Leu Thr Glu Ile
Ser Lys Leu Val Glu Asn Tyr Ser Lys Ile Lys Ile Pro Glu Asn Lys
Pro Leu Val Gly Glu Met Val Phe Tyr His Glu Ser Gly Ile His Val
Asp Ala Val Leu Glu Asn Pro Leu Thr Tyr Glu Pro Phe Leu Pro Glu
Lys Ile Gly Gln Lys Arg Lys Ile Ile Leu Gly Lys His Ser Gly Cys
Arg Ala Val Ala His Arg Leu Gln Glu Leu Gly Leu Glu Ala Ser Arg
Asp Glu Leu Trp Glu Ile Val Lys Lys Thr Lys Glu Thr Arg Glu Asp
Gly Thr Glu Ile Ser Asp Glu Val Phe Lys Asn Ile Ala Glu Lys Ile
Ile Lys
SEQ ID NO: 7
PRT - Methanococcus maripaludis C5
Met Asp Trp Lys Ala Val Ser Pro Tyr Asn Pro Lys Leu Asn Leu Lys
Asp Cys Tyr Leu Tyr Asp Thr Thr Leu Arg Asp Gly Glu Gln Thr Pro
Gly Val Cys Phe Thr His Asp Gln Lys Leu Glu Ile Ala Lys Lys Leu
Asp Glu Leu Lys Ile Lys Gln Ile Glu Ala Gly Phe Pro Ile Val Ser
Glu Asn Glu Arg Lys Ala Ile Lys Ser Ile Thr Gly Glu Gly Leu Asn
Ala Gln Ile Leu Ala Leu Ser Arg Val Leu Lys Glu Asp Ile Asp Lys
Ala Ile Glu Cys Asp Val Asp Gly Ile Ile Thr Phe Ile Ala Ala Ser
Pro Met His Leu Lys Tyr Lys Leu His Lys Ser Leu Asp Glu Val Glu
Glu Met Gly Met Lys Ala Val Glu Tyr Ala Lys Asp His Gly Leu Phe
Val Ala Phe Ser Ala Glu Asp Ala Thr Arg Thr Pro Val Glu Asp Leu
Ile Arg Ile His Lys Asn Ala Glu Glu His Gly Ala Asn Arg Val His
Ile Ala Asp Thr Leu Gly Cys Ala Thr Pro Gln Ala Met Tyr His Ile
Cys Ser Glu Leu Ser Ser Asn Leu Lys Lys Ala His Ile Gly Val His
Cys His Asn Asp Phe Gly Phe Ala Val Ile Asn Ser Ile Tyr Gly Leu
Ile Gly Gly Ala Lys Ala Val Ser Thr Thr Val Asn Gly Ile Gly Glu
Arg Ala Gly Asn Ala Ala Ile Glu Glu Ile Val Met Ala Leu Lys Val
Leu Tyr Asp His Asp Met Gly Leu Asn Thr Glu Ile Leu Thr Glu Ile
Ser Lys Leu Val Glu Asn Tyr Ser Lys Ile Arg Ile Pro Glu Asn Lys
Pro Leu Val Gly Glu Met Ala Phe Tyr His Glu Ser Gly Ile His Val
Asp Ala Val Leu Glu Asn Pro Leu Thr Tyr Glu Pro Phe Leu Pro Glu
Lys Ile Gly Gln Lys Arg Lys Ile Ile Leu Gly Lys His Ser Gly Cys
Arg Ala Val Ala His Arg Leu Gln Glu Leu Gly Leu Glu Ala Ser Arg
Glu Glu Leu Trp Glu Ile Val Lys Lys Thr Lys Glu Thr Arg Glu Glu
Gly Thr Glu Ile Ser Asp Glu Val Phe Lys Asn Ile Val Asp Lys Ile
Ile Lys
SEQ ID NO: 8
PRT - Methanococcus maripaludis C7
Met Asp Trp Lys Ala Val Ser Pro Tyr Asn Pro Lys Leu Asp Leu Lys
Asp Cys Tyr Leu Tyr Asp Thr Thr Leu Arg Asp Gly Glu Gln Thr Pro
Gly Val Cys Phe Thr His Asp Gln Lys Leu Glu Ile Ala Lys Lys Leu
Asp Glu Leu Lys Ile Lys Gln Ile Glu Ala Gly Phe Pro Ile Val Ser
Glu Asn Glu Arg Lys Cys Ile Lys Ser Ile Thr Gly Glu Gly Leu Asn
Ala Gln Ile Leu Ala Leu Ser Arg Val Leu Lys Glu Asp Ile Asp Lys
Ala Ile Glu Cys Asp Val Asp Gly Ile Ile Thr Phe Ile Ala Ala Ser
Pro Met His Leu Lys Tyr Lys Leu His Lys Ser Leu Asp Glu Val Glu
Glu Met Gly Met Lys Ala Val Glu Tyr Ala Lys Asp His Gly Leu Phe
Val Ala Phe Ser Ala Glu Asp Ala Thr Arg Thr Pro Ile Glu Asp Ile
Ile Arg Ile His Lys Asn Ala Glu Glu His Gly Ala Asp Arg Val His
Ile Ala Asp Thr Leu Gly Cys Ala Thr Pro Gln Ser Met Tyr Tyr Ile
Cys Ser Glu Leu Ser Lys His Leu Lys Lys Ala His Ile Gly Val His
Cys His Asn Asp Phe Gly Phe Ala Val Ile Asn Ser Ile Tyr Gly Leu
Leu Gly Gly Ala Lys Ala Val Ser Thr Thr Val Asn Gly Ile Gly Glu
Arg Ala Gly Asn Ala Ala Ile Glu Glu Ile Val Met Ala Leu Lys Val
Leu Tyr Asp Tyr Asp Met Gly Leu Asn Thr Glu Ile Leu Thr Glu Met
Ser Lys Leu Val Glu Lys Tyr Ser Lys Ile Arg Ile Pro Glu Asn Lys
Pro Leu Val Gly Glu Met Ala Phe Tyr His Glu Ser Gly Ile His Val
Asp Ala Val Leu Glu Asn Pro Leu Thr Tyr Glu Pro Phe Leu Pro Glu
Lys Ile Gly Gln Lys Arg Lys Ile Ile Leu Gly Lys His Ser Gly Cys
Arg Ala Val Ala His Arg Leu Gln Glu Leu Gly Leu Glu Thr Ser Arg
Asn Glu Leu Trp Glu Ile Val Lys Lys Thr Lys Glu Thr Arg Glu Glu
Gly Thr Glu Ile Ser Asp Glu Val Phe Lys Asn Ile Val Asp Lys Ile
Ile Lys
SEQ ID NO: 9
PRT - Methanospaera stadtmanae DSM 3091
Met Gly Leu Ser Asp Leu His Leu Glu Val Lys Ile Asn Lys Pro Arg
Asp Val Val Asn Gln Ile Cys Met Asp Ala Ile Asp Tyr Gly Lys Asp
His Gly Leu Phe Val Ala Phe Ser Ala Glu Asp Ala Thr Arg Thr Glu
Leu Pro Lys Leu Leu Asp Val Tyr Lys Gln Ala Gln Asp His Gly Ala
Asp Arg Ile His Ile Ala Asp Thr Thr Gly Ser Ile Asn Pro Tyr Ala
Thr Gln Tyr Leu Val Lys Asn Ile Lys Lys Glu Ile Asp Thr Glu Ile
Ala Leu His Cys His Asn Asp Phe Gly Phe Ala Val Ala Asn Ser Ile
Ala Gly Leu Phe Glu Gly Ala Thr Ala Ile Ser Thr Thr Val Asn Gly
Ile Gly Glu Arg Ala Gly Asn Ala Ser Leu Glu Glu Leu Ile Met Ser
Leu Lys Leu Leu Tyr Asn Lys Asp Leu Gly Phe Lys Thr Glu Val Ile
Tyr Glu Leu Ser Gln Leu Val Ser Lys Tyr Ser Lys Ile Pro Ile Ser
Asp Ser Lys Ala Ile Val Gly Asn Asn Val Phe Arg His Glu Ser Gly
Ile His Val Asp Ala Ile Val Lys Asn Pro Leu Ala Tyr Glu Pro Phe
Ile Pro Glu Met Ile Gly Thr Lys Arg Gln Ile Val Leu Gly Lys His
Ser Gly Lys Ser Ala Val Ile Glu Lys Leu Asp Thr Leu Asn Ile Lys
Val Asp Asp Thr Gln Leu Ser Gln Ile Val Ser Leu Val Lys Gln Glu
Arg Glu Arg Gly Glu Glu Ile Thr Asn Asn Lys Phe Asp Glu Ile Leu
Glu Lys Val Asn Ile Lys Arg
SEQ ID NO: 10
PRT - Methanopyrus kandleri AV19
Met Gln Ser Pro Tyr Val Arg Glu Ala Val Arg Glu Met Asp Leu Pro
Asp Glu Val Ile Val Tyr Asp Thr Thr Leu Arg Asp Gly Glu Gln Thr
Pro Gly Val Ser Phe Thr Pro Glu Gln Lys Leu Glu Ile Ala His Leu
Leu Asp Glu Leu Gly Val Gln Gln Ile Glu Ala Gly Phe Pro Val Val
Ser Glu Gly Glu Arg Asp Ala Val Arg Arg Ile Ala His Glu Gly Leu
Asn Ala Asp Ile Leu Cys Leu Ala Arg Thr Leu Arg Gly Asp Val Asp
Ala Ala Leu Asp Cys Asp Val Asp Gly Val Ile Thr Phe Ile Ala Thr
Ser Glu Leu His Leu Lys His Lys Leu Arg Met Ser Arg Glu Glu Val
Leu Glu Arg Ile Ala Asp Thr Val Glu Tyr Ala Lys Asp His Gly Leu
Trp Val Ala Phe Ser Ala Glu Asp Gly Thr Arg Thr Glu Phe Glu Phe
Leu Glu Arg Val Tyr Arg Thr Ala Glu Glu Cys Gly Ala Asp Arg Val
His Ala Thr Asp Thr Val Gly Val Met Ile Pro Ala Ala Met Arg Leu
Phe Val Ala Lys Ile Arg Glu Val Val Asp Leu Pro Ile Gly Val His
Cys His Asp Asp Phe Gly Met Ala Val Ala Asn Ser Leu Ala Ala Val
Glu Ala Gly Ala Gln Ala Ile Ser Thr Thr Val Asn Gly Ile Gly Glu
Arg Ala Gly Asn Ala Ala Leu Glu Glu Val Ile Met Ala Leu Lys Glu
Leu Tyr Gly Ile Asp Pro Gly Phe Asn Thr Glu Val Leu Ala Glu Leu
Ser Arg Lys Val Ser Glu Tyr Ser Gly Ile Asp Val Pro Pro Asn Lys
Ala Val Val Gly Glu Asn Ala Phe Arg His Glu Ser Gly Ile His Val
Ala Ala Val Leu Glu Glu Pro Arg Thr Tyr Glu Pro Ile Asp Pro Lys
Glu Val Gly Met Asn Arg Lys Ile Val Leu Gly Lys His Thr Gly Arg
Lys Ala Val Val Ala Lys Leu Glu Glu Leu Gly Val Glu Pro Glu Glu
Glu Ile Val Glu Glu Val Leu Lys Arg Ile Lys Ala Leu Gly Asp Arg
Arg Val Arg Val Thr Asp Ser Lys Leu Glu Glu Ile Val Arg Asn Val
Leu Glu Ser Arg Gly Asp Arg Asp Asp Pro Gly Ser Arg
SEQ ID NO: 11
PRT - Methanobrevibacter smithii ATCC35061
Met Gln Tyr Tyr Ile Ser His Tyr Asn Lys Glu Pro Glu Leu Asn Phe
Pro Asp Glu Ile Thr Val Tyr Asp Thr Thr Leu Arg Asp Gly Glu Gln
Thr Pro Gly Val Cys Phe Ser Pro Glu Glu Lys Leu Glu Ile Ala Lys
Lys Leu Asp Glu Val Lys Ile Lys Gln Ile Glu Ala Gly Phe Pro Ile
Val Ser Lys Lys Glu Gln Glu Ser Val Lys Ala Ile Thr Ser Glu Gly
Leu Asn Ala Gln Ile Ile Ser Leu Ser Arg Thr Lys Lys Glu Asp Ile
Asp Ala Ala Leu Asp Cys Asp Val Asp Gly Val Ile Thr Phe Met Gly
Thr Ser Asp Ile His Leu Glu His Lys Met His Ile Gly Arg Gln Glu
Ala Leu Asn Thr Cys Met Asn Ala Ile Glu Tyr Ala Lys Asp His Gly
Leu Phe Val Ala Phe Ser Ala Glu Asp Ala Thr Arg Thr Asp Leu Asp
Phe Leu Lys Arg Ile Tyr Asn Lys Ala Glu Ser Tyr Gly Ala Asp Arg
Val His Ile Ala Asp Thr Thr Gly Ala Ile Thr Pro Gln Gly Ile Thr
Tyr Leu Val Lys Glu Leu Lys Lys Asp Val Asn Ile Asp Ile Ala Leu
His Cys His Asn Asp Phe Gly Leu Ala Val Ile Asn Ser Ile Ser Gly
Val Leu Ala Gly Ala Asn Gly Ile Ser Thr Thr Val Asn Gly Ile Gly
Glu Arg Ala Gly Asn Ala Ser Leu Glu Glu Val Ile Met Ser Leu Lys
Leu Leu Tyr Gly Lys Asp Leu Gly Phe Lys Thr Lys His Ile Lys Glu
Leu Ser Glu Leu Val Ser Lys Ala Ser Gly Leu Pro Val Pro Tyr Asn
Lys Pro Val Val Gly Asn Asn Val Phe Arg His Glu Ser Gly Ile His
Val Asp Ala Val Ile Glu Glu Pro Leu Cys Tyr Glu Pro Tyr Ile Pro
Glu Leu Val Gly Gln Lys Arg Gln Leu Val Leu Gly Lys His Ser Gly
Cys Arg Ala Val Arg Ala Lys Leu Asn Glu Cys Asp Leu Asp Val Ser
Asp Asp Thr Leu Ile Glu Ile Val Lys Lys Val Lys Lys Ser Arg Glu
Glu Gly Thr Tyr Ile Asn Asp Asp Val Phe Lys Glu Ile Val Lys Ser
Cys Asn Tyr Lys Lys Glu
SEQ ID NO: 12
PRT - Methanococcus vannielii SB
Met Asp Trp Lys Glu Val Ser Gln Tyr Asn Pro Lys Leu Asp Leu Lys
Glu Cys Tyr Val Tyr Asp Thr Thr Leu Arg Asp Gly Glu Gln Thr Pro
Gly Val Cys Phe Thr Gly Asn Gln Lys Leu Glu Ile Ala Lys Lys Leu
Asp Asp Leu Gly Ile Lys Gln Ile Glu Ala Gly Phe Pro Thr Val Ser
Glu Asn Glu Arg Lys Cys Ile Lys Ser Ile Ser Ser Glu Gly Leu Asn
Ala Asp Ile Leu Ala Leu Ser Arg Val Leu Lys Glu Asp Ile Asp Arg
Ala Ile Glu Cys Asp Val Asp Gly Ile Ile Thr Phe Val Ala Thr Ser
Pro Met His Leu Lys Tyr Lys Leu His Lys Ser Phe Glu Glu Val Glu
Glu Met Gly Met Lys Ala Ile Glu Tyr Ala Lys Asp His Gly Leu Phe
Val Ala Phe Ser Ala Glu Asp Ala Thr Arg Thr Ser Ile Glu Asn Ile
Ile Lys Ile His Lys Asn Ala Glu Asp Tyr Gly Ala Asp Arg Val His
Ile Ala Asp Thr Leu Gly Cys Ala Thr Pro Gln Ser Met Tyr Gln Ile
Cys Ser Glu Leu Asn Lys Ser Leu Lys Lys Ala His Ile Gly Val His
Cys His Asn Asp Phe Gly Phe Ala Ala Ile Asn Ser Ile Tyr Gly Leu
Met Gly Gly Ala Lys Ala Val Ser Thr Thr Val Asn Gly Ile Gly Glu
Arg Ala Gly Asn Ala Ala Leu Glu Glu Val Val Met Ala Leu Lys Val
Leu Tyr Asn Tyr Asp Met Gly Leu Asn Thr Glu Leu Ile Met Glu Thr
Ser Lys Leu Val Glu Thr Tyr Ser Lys Ile Lys Val Pro Glu Asn Lys
Pro Leu Val Gly Glu Met Val Phe Tyr His Glu Ser Gly Ile His Val
Asp Ala Val Leu Glu Asn Pro Leu Thr Tyr Glu Pro Phe Leu Pro Glu
Lys Ile Gly Gln Lys Arg Lys Ile Val Leu Gly Lys His Ser Gly Cys
Arg Ala Val Ala Tyr Arg Leu Asn Glu Leu Gly Phe Glu Ala Thr Arg
Asp Glu Leu Trp Glu Ile Val Lys Lys Thr Lys Glu Thr Arg Glu Gln
Gly Thr Glu Ile Ser Asp Glu Val Phe Lys Asn Ile Val Thr His Ile
Leu Asn
SEQ ID NO: 13
PRT - Methanococcus aeolicus Nankai 3
Met Asn Trp Lys Glu Val Cys Gln Tyr Asn Pro Lys Leu Asn Leu Glu
Asp Cys Tyr Ile Tyr Asp Thr Thr Leu Arg Asp Gly Glu Gln Thr Pro
Gly Val Cys Phe Ser Met Glu Gln Lys Leu Asp Ile Ala Lys Lys Leu
Asp Glu Leu Gly Val Lys Gln Ile Glu Ala Gly Phe Pro Ala Val Ser
Lys Ser Glu Ile Glu Asn Val Lys Lys Ile Ala Asn Glu Gly Leu Asn
Ala Glu Ile Leu Ala Leu Ser Arg Ala Leu Gln Gly Asp Ile Asp Lys
Ala Leu Ser Cys Asp Val Asp Gly Ile Ile Thr Phe Ile Ala Ala Ser
Pro Leu His Leu Lys Tyr Lys Leu His Lys Ser Ile Glu Glu Val Glu
Glu Met Gly Met Lys Ala Val Glu Tyr Ala Lys Asp His Gly Leu Phe
Val Ala Phe Ser Ala Glu Asp Ala Thr Arg Thr Pro Ile Glu Asp Leu
Val Arg Ile His Lys Asn Ala Glu Glu His Gly Ala Asp Arg Val His
Ile Ala Asp Thr Thr Gly Cys Gly Thr Pro Gln Ser Ile Gln Tyr Ile
Cys Ser Glu Leu Ser Asn Asn Leu Lys Lys Ala His Ile Gly Val His
Cys His Asn Asp Phe Gly Leu Ala Val Ile Asn Ser Ile Tyr Gly Leu
Leu Gly Gly Ala Lys Ala Ala Ser Thr Thr Val Asn Gly Ile Gly Glu
Arg Ala Gly Asn Ala Pro Leu Glu Glu Leu Leu Leu Thr Met Asn Val
Leu Tyr Asp Val Lys Thr Asp Leu Asn Ile Ser Ile Ile Lys Glu Leu
Ser Thr Met Val Glu Asn Tyr Ser Gly Ile Lys Ile Pro Val Asn Lys
Pro Ile Val Gly Asp Lys Val Phe Tyr His Glu Ser Gly Ile His Val
Asp Ala Val Ile Glu Asn Pro Leu Thr Tyr Glu Pro Phe Leu Pro Glu
Arg Ile Gly Gln Lys Arg Glu Ile Val Leu Gly Lys His Ser Gly Cys
Ser Ala Val Glu Ser Lys Leu Lys Glu Leu Gly Leu Glu Val Pro Lys
Asp Arg Ile Trp Asp Leu Val Lys Lys Val Lys Thr Thr Arg Glu Gly
Gly Glu Asp Ile Asp Asp Glu Met Phe Ile Lys Ile Val Asp Ile Ile
Asn Lys Gln
SEQ ID NO: 14
PRT - Methanocaldococcus jannashii DSM2661
Met Thr Leu Val Glu Lys Ile Leu Ser Lys Lys Val Gly Tyr Glu Val
Cys Ala Gly Asp Ser Ile Glu Val Glu Val Asp Leu Ala Met Thr His
Asp Gly Thr Thr Pro Leu Ala Tyr Lys Ala Leu Lys Glu Met Ser Asp
Ser Val Trp Asn Pro Asp Lys Ile Val Val Ala Phe Asp His Asn Val
Pro Pro Asn Thr Val Lys Ala Ala Glu Met Gln Lys Leu Ala Leu Glu
Phe Val Lys Arg Phe Gly Ile Lys Asn Phe His Lys Gly Gly Glu Gly
Ile Cys His Gln Ile Leu Ala Glu Asn Tyr Val Leu Pro Asn Met Phe
Val Ala Gly Gly Asp Ser His Thr Cys Thr His Gly Ala Phe Gly Ala
Phe Ala Thr Gly Phe Gly Ala Thr Asp Met Ala Tyr Ile Tyr Ala Thr
Gly Glu Thr Trp Ile Lys Val Pro Lys Thr Ile Arg Val Asp Ile Val
Gly Lys Asn Glu Asn Val Ser Ala Lys Asp Ile Val Leu Arg Val Cys
Lys Glu Ile Gly Arg Arg Gly Ala Thr Tyr Met Ala Ile Glu Tyr Gly
Gly Glu Val Val Lys Asn Met Asp Met Asp Gly Arg Leu Thr Leu Cys
Asn Met Ala Ile Glu Met Gly Gly Lys Thr Gly Val Ile Glu Ala Asp
Glu Ile Thr Tyr Asp Tyr Leu Lys Lys Glu Arg Gly Leu Ser Asp Glu
Asp Ile Ala Lys Leu Lys Lys Glu Arg Ile Thr Val Asn Arg Asp Glu
Ala Asn Tyr Tyr Lys Glu Ile Glu Ile Asp Ile Thr Asp Met Glu Glu
Gln Val Ala Val Pro His His Pro Asp Asn Val Lys Pro Ile Ser Asp
Val Glu Gly Thr Glu Ile Asn Gln Val Phe Ile Gly Ser Cys Thr Asn
Gly Arg Leu Ser Asp Leu Arg Glu Ala Ala Lys Tyr Leu Lys Gly Arg
Glu Val His Lys Asp Val Lys Leu Ile Val Ile Pro Ala Ser Lys Lys
Val Phe Leu Gln Ala Leu Lys Glu Gly Ile Ile Asp Ile Phe Val Lys
Ala Gly Ala Met Ile Cys Thr Pro Gly Cys Gly Pro Cys Leu Gly Ala
His Gln Gly Val Leu Ala Glu Gly Glu Ile Cys Leu Ser Thr Thr Asn
Arg Asn Phe Lys Gly Arg Met Gly His Ile Asn Ser Tyr Ile Tyr Leu
Ala Ser Pro Lys Ile Ala Ala Ile Ser Ala Val Lys Gly Tyr Ile Thr
Asn Lys Leu Asp
SEQ ID NO: 15
PRT - Methanothermobacter thermoautotropicum DH
Met Val Lys Met Asn Met Thr Glu Lys Ile Leu Ala Glu Ala Ala Gly
Leu Arg Glu Val Thr Pro Gly Glu Ile Ile Glu Ala Arg Val Asp Leu
Ala Met Thr His Asp Gly Thr Ser Pro Pro Thr Ile Arg Thr Phe Arg
Asp Ile Ala Ser Arg Gly Gly Pro Ala Arg Val Trp Asp Pro Glu Arg
Ile Val Met Val Phe Asp His Asn Val Pro Ala Asn Thr Ile Gly Ala
Ala Glu Phe Gln Arg Val Thr Arg Glu Phe Ala Arg Glu Gln Gly Ile
Val Asn Ile Phe Gln Asn Ala Ala Gly Ile Cys His Gln Val Leu Pro
Glu Arg Gly Phe Val Arg Pro Gly Met Val Ile Val Gly Ala Asp Ser
His Thr Cys Thr Tyr Gly Ala Phe Gly Ala Phe Ala Thr Gly Met Gly
Ala Thr Asp Met Ala Met Val Phe Ala Thr Gly Lys Thr Trp Phe Met
Val Pro Glu Ala Met Arg Ile Glu Val Thr Gly Glu Pro Glu Gly His
Val Tyr Ala Lys Asp Val Ile Leu His Ile Ile Gly Glu Ile Gly Val
Asp Gly Ala Thr Tyr Arg Ser Val Glu Phe Thr Gly Asp Thr Ile Glu
Ser Met Asp Val Ser Gly Arg Met Thr Ile Cys Asn Met Ala Val Glu
Met Gly Ala Lys Asn Gly Ile Met Glu Pro Asn Arg Gln Thr Leu Asp
Tyr Val Arg Ala Arg Thr Gly Arg Glu Phe Arg Val Tyr Ser Ser Asp
Glu Asp Ser Gln Tyr Leu Glu Asp His His Phe Asp Val Ser Asp Leu
Glu Pro Gln Val Ala Cys Pro Asp Asp Val Asp Asn Val Tyr Pro Val
His Arg Val Glu Gly Thr His Ile Asp Glu Ala Phe Leu Gly Ser Cys
Thr Asn Gly Arg Tyr Glu Asp Leu Lys Ile Ala Ala Glu Val Ile Gly
Asp Arg Arg Val His Glu Asp Val Arg Phe Ile Val Ser Pro Ala Ser
Arg Glu Ile Tyr Leu Lys Ala Leu Glu Asp Gly Ile Ile Glu Thr Phe
Ile Arg Ala Gly Ala Ile Val Cys Asn Pro Gly Cys Gly Pro Cys Leu
Gly Ala His Met Gly Val Leu Ala Pro Gly Glu Val Ser Ile Ala Thr
Thr Asn Arg Asn Phe Arg Gly Arg Met Gly Asp Pro Ala Ser Ser Val
Tyr Leu Ala Asn Pro Ala Val Val Ala Glu Ser Ala Ile Glu Gly Val
Ile Ser Ala Pro Gln Gln Glu Ala Gly Asn Gly Cys
SEQ ID NO: 16
PRT - Methanococcus maripaludis S2
Met Thr Leu Ala Glu Lys Ile Ile Ser Lys Asn Val Gly Lys Asn Val
Tyr Ala Lys Asp Ser Val Glu Ile Ser Val Asp Ile Ala Met Thr His
Asp Gly Thr Thr Pro Leu Thr Val Lys Ala Phe Glu Gln Ile Ser Asp
Lys Val Trp Asp Asn Glu Lys Ile Val Ile Ile Phe Asp His Asn Ile
Pro Ala Asn Thr Ser Lys Ala Ala Asn Met Gln Val Ile Thr Arg Glu
Phe Ile Lys Lys Gln Gly Ile Lys Asn Tyr Tyr Leu Asp Gly Glu Gly
Ile Cys His Gln Val Leu Pro Glu Lys Gly His Val Lys Pro Asn Met
Ile Ile Ala Gly Ala Asp Ser His Thr Cys Thr His Gly Ala Phe Gly
Ala Phe Ala Thr Gly Phe Gly Ala Thr Asp Met Gly Tyr Val Tyr Ala
Thr Gly Lys Thr Trp Leu Arg Val Pro Glu Thr Ile Arg Val Asn Val
Thr Gly Glu Asn Glu Asn Ile Ser Gly Lys Asp Ile Ile Leu Lys Thr
Cys Lys Glu Val Gly Arg Arg Gly Ala Thr Tyr Met Ser Leu Glu Tyr
Gly Gly Asn Ala Val His Asn Leu Ser Met Asp Glu Arg Met Val Leu
Ser Asn Met Ala Ile Glu Met Gly Gly Lys Ala Gly Ile Ile Glu Ala
Asp Asp Thr Thr Tyr Arg Tyr Leu Glu Asn Ala Gly Val Ser Arg Glu
Glu Ile Leu Glu Leu Lys Lys Asn Lys Ile Thr Val Asp Glu Ser Glu
Glu Asp Tyr Tyr Lys Thr Ile Glu Phe Asp Ile Thr Gly Met Glu Glu
Gln Val Ala Cys Pro His His Pro Asp Asn Val Lys Gly Val Ser Glu
Val Glu Gly Thr Glu Leu Asn Gln Val Phe Ile Gly Ser Cys Thr Asn
Gly Arg Leu Asn Asp Leu Arg Ile Ala Ala Lys Tyr Leu Lys Gly Lys
Lys Val Asn Glu Asn Thr Arg Leu Ile Val Ile Pro Ala Ser Lys Ser
Ile Phe Lys Glu Ala Leu Asn Glu Gly Leu Ile Asp Ile Phe Val Asp
Ser Gly Ala Leu Ile Cys Thr Pro Gly Cys Gly Pro Cys Leu Gly Ala
His Gln Gly Val Leu Gly Asp Gly Glu Val Cys Leu Ala Thr Thr Asn
Arg Asn Phe Lys Gly Arg Met Gly Asn Thr Asn Ala Gln Val Tyr Leu
Ser Ser Pro Lys Ile Ala Ala Lys Ser Ala Val Lys Gly Tyr Ile Thr
Asn Glu
SEQ ID NO: 17
PRT - Methanococcus maripaludis C5
Met Thr Leu Ala Glu Lys Ile Ile Ser Lys Asn Val Gly Lys Asn Val
Tyr Ala Gly Asp Ser Val Glu Ile Asp Val Asp Val Ala Met Thr His
Asp Gly Thr Thr Pro Leu Thr Val Lys Ala Phe Glu Gln Ile Ser Asp
Lys Val Trp Asp Asn Glu Lys Ile Val Ile Ile Phe Asp His Asn Ile
Pro Ala Asn Thr Ser Lys Ala Ala Asn Met Gln Val Ile Thr Arg Glu
Phe Ile Lys Lys Gln Gly Ile Lys Asn Tyr Tyr Leu Asp Gly Glu Gly
Ile Cys His Gln Val Leu Pro Glu Lys Gly His Val Lys Pro Asn Met
Ile Ile Ala Gly Ala Asp Ser His Thr Cys Thr His Gly Ala Phe Gly
Ala Phe Ala Thr Gly Phe Gly Ala Thr Asp Met Gly Tyr Val Tyr Ala
Thr Gly Lys Thr Trp Leu Arg Val Pro Glu Thr Ile Gln Val Asn Val
Thr Gly Glu Asn Glu Asn Ile Ser Gly Lys Asp Ile Ile Leu Lys Thr
Cys Lys Glu Val Gly Arg Arg Gly Ala Thr Tyr Leu Ser Leu Glu Tyr
Gly Gly Asn Ala Val Gln Asn Leu Asp Met Asp Glu Arg Met Val Leu
Ser Asn Met Ala Ile Glu Met Gly Gly Lys Ala Gly Ile Ile Glu Ala
Asp Asp Thr Thr Tyr Lys Tyr Leu Glu Asn Ala Gly Val Ser Arg Glu
Glu Ile Leu Asn Leu Lys Lys Asn Lys Ile Lys Val Asn Glu Ser Glu
Glu Asn Tyr Tyr Lys Thr Phe Glu Phe Asp Ile Thr Asp Met Glu Glu
Gln Ile Ala Cys Pro His His Pro Asp Asn Val Lys Gly Val Ser Glu
Val Ser Gly Ile Glu Leu Asp Gln Val Phe Ile Gly Ser Cys Thr Asn
Gly Arg Leu Asn Asp Leu Arg Ile Ala Ala Lys His Leu Lys Gly Lys
Lys Val Asn Glu Ser Thr Arg Leu Ile Val Ile Pro Ala Ser Lys Ser
Ile Phe Lys Glu Ala Leu Lys Glu Gly Leu Ile Asp Thr Phe Val Asp
Ser Gly Ala Leu Ile Cys Thr Pro Gly Cys Gly Pro Cys Leu Gly Ala
His Gln Gly Val Leu Gly Asp Gly Glu Val Cys Leu Ala Thr Thr Asn
Arg Asn Phe Lys Gly Arg Met Gly Asn Thr Lys Ser Glu Val Tyr Leu
Ser Ser Pro Ala Ile Ala Ala Lys Ser Ala Val Lys Gly Tyr Ile Thr
Asn Glu
SEQ ID NO: 18
PRT - Methanococcus maripaludis C7
Met Thr Leu Ala Glu Lys Ile Ile Ser Lys Asn Val Gly Lys Asn Val
Tyr Ala Gly Asp Ser Val Glu Ile Asp Val Asp Ile Ala Met Thr His
Asp Gly Thr Thr Pro Leu Thr Val Lys Ala Phe Glu Gln Ile Ser Asp
Lys Val Trp Asp Asn Glu Lys Ile Val Ile Ile Phe Asp His Asn Ile
Pro Ala Asn Thr Ser Lys Ala Ala Asn Met Gln Val Ile Thr Arg Glu
Phe Ile Lys Lys His Gly Ile Lys Asn Tyr Tyr Leu Asp Gly Glu Gly
Ile Cys His Gln Val Leu Pro Glu Lys Gly His Val Lys Pro Asn Met
Ile Ile Ala Gly Ala Asp Ser His Thr Cys Thr His Gly Ala Phe Gly
Ala Phe Ala Thr Gly Phe Gly Ala Thr Asp Met Gly Phe Val Tyr Ala
Thr Gly Lys Thr Trp Leu Arg Val Pro Glu Thr Ile Arg Val Asn Val
Thr Gly Glu Asn Glu Asn Ile Ser Gly Lys Asp Ile Ile Leu Lys Thr
Cys Lys Glu Val Gly Arg Ser Gly Ala Thr Tyr Met Ser Leu Glu Tyr
Gly Gly Asn Ala Val Gln Asn Leu Glu Met Asn Glu Arg Met Val Leu
Ser Asn Met Ala Ile Glu Met Gly Gly Lys Ala Gly Ile Ile Glu Ala
Asp Asp Thr Thr Tyr Lys Tyr Leu Glu Asn Ala Gly Val Ser Arg Glu
Glu Ile Leu Asn Leu Lys Lys Asn Lys Ile Thr Val Asn Glu Ser Glu
Glu Asn Tyr Tyr Lys Thr Ile Glu Phe Asp Ile Thr Asp Met Glu Glu
Gln Ile Ala Cys Pro His Asn Pro Asp Asn Val Lys Gly Val Ser Glu
Val Ser Gly Thr Glu Leu Asp Gln Val Phe Ile Gly Ser Cys Thr Asn
Gly Arg Leu Asn Asp Leu Arg Ile Ala Ala Lys Tyr Leu Lys Gly Lys
Lys Val Asn Glu Asn Thr Arg Leu Ile Val Ile Pro Ala Ser Lys Ser
Ile Phe Ala Gly Ala Leu Lys Glu Gly Leu Ile Asp Ile Phe Val Glu
Ser Gly Ala Leu Ile Cys Thr Pro Gly Cys Gly Pro Cys Leu Gly Ala
His Gln Gly Val Leu Gly Asp Gly Glu Val Cys Leu Ala Thr Thr Asn
Arg Asn Phe Lys Gly Arg Met Gly Asn Thr Lys Ala Glu Val Tyr Leu
Ser Ser Pro Lys Ile Ala Ala Lys Ser Ala Val Lys Gly Tyr Ile Thr
Asn Glu
SEQ ID NO: 19
PRT - Methanospaera stadtmanae DSM 3091
Met Asn Ile Ser Glu Lys Ile Leu Ala Lys Ala Ser Asn Lys Glu Glu
Val Ser Pro Gly Asp Thr Ile Thr Ala Asn Ile Asp Val Ala Met Ser
His Asp Gly Thr Ser Pro Pro Thr Ile Lys Val Phe Glu Lys Ile Ala
Asp Lys Val Trp Asp Pro Glu Lys Ile Val Leu Val Phe Asp His Val
Ile Pro Ala Asn Thr Ile Gly Ser Ala Glu Phe Gln Gln Val Val Arg
Glu Phe Gly Lys Lys Gln Lys Ile Pro Asn Met Tyr Ile Gln Gly Glu
Gly Val Cys His Glu Val Leu Pro Asp Tyr Gly His Val Lys Pro Ser
Thr Val Ile Val Gly Ala Asp Ser His Thr Cys Thr Tyr Gly Ala Phe
Gly Ala Phe Ser Thr Gly Leu Gly Ala Thr Asp Leu Ala Met Val Tyr
Ala Thr Gly Gln Thr Trp Phe Asn Val Pro Glu Ser Leu Lys Ile Asn
Val Asn Gly Thr Leu Asn Glu Asn Val Tyr Ser Lys Asp Val Ile Leu
Lys Ile Ile Lys Glu Leu Gly Ala Tyr Gly Ala Thr Tyr Lys Ser Leu
Glu Phe His Gly Asp Thr Ile Asp Asn Met Ser Val Ala Ser Arg Leu
Thr Met Thr Asn Met Ala Ile Glu Cys Gly Ala Lys Asn Gly Ile Met
Val Pro Asn Lys Gln Thr Lys Glu Tyr Leu Ser Gln Arg Gly Ile Thr
Asp Tyr Thr Ile Thr Thr Ala Ser Lys Asp Ala Glu Tyr Glu Lys Ile
Tyr Asp Phe Asp Val Asp Asp Leu Gln Pro Gln Ile Ala Cys Pro His
Asn Val Asp Asn Val Glu Asp Ile Asp Lys Val Ala Gly Thr His Ile
Asp Gln Ala Val Leu Gly Ser Cys Thr Asn Gly Arg Tyr Glu Asp Leu
Leu Gln Ala Ala Glu Val Ile Glu Gly His Lys Ile His Glu Asp Val
Glu Leu Leu Val Phe Pro Ala Ser Arg His Val Tyr Glu Lys Ala Ile
Glu Thr Gly Val Ile Gln Thr Leu Leu Lys Ser Asn Ala Ile Ile Cys
Asn Pro Gly Cys Gly Pro Cys Leu Gly Ala His Met Gly Val Met Thr
Asp Asp Met Thr Cys Ile Ser Thr Thr Asn Arg Asn Phe Leu Gly Arg
Met Gly Ser Ala Lys Ser Tyr Val Tyr Leu Ser Asn Pro Ala Val Val
Ala Ala Ser Ala Ile Lys Gly Glu Ile Thr Asn Pro Ser Glu Ile
SEQ ID NO: 20
PRT - Methanopyrus kandleri AV19
Met Gly Lys Thr Met Ala Glu Lys Ile Leu Ser Arg Ala Ser Gly Glu
Asp Ala Glu Ala Gly Asp Ile Val Val Ala Asn Ile Asp Val Ala Met
Val His Asp Ile Thr Gly Pro Ile Thr Val Gln Arg Leu Glu Glu Met
Gly Val Glu Arg Val Trp Asp Pro Ser Lys Ile Val Val Leu Phe Asp
His Gln Val Pro Ala Asp Ser Val Glu Ala Ala Glu Asn His Lys Ile
Met Arg Glu Phe Val Glu Glu Gln Gly Ile Glu His Phe Tyr Asp Val
Arg Glu Gly Val Cys His Gln Val Leu Pro Glu Lys Gly His Val Arg
Pro Gly Asp Val Ile Val Gly Ala Asp Ser His Thr Cys Thr His Gly
Ala Leu Gly Ala Phe Ala Thr Gly Ile Gly Ser Thr Asp Met Ala Ala
Val Phe Ala Thr Gly Lys Leu Trp Phe Arg Val Pro Glu Thr Tyr Arg
Val Glu Ile Thr Gly Glu Leu Pro Glu Gly Val Tyr Ala Lys Asp Val
Val Leu Lys Val Thr Gly Glu Ile Gly Ala Asp Gly Ala Thr Tyr Met
Ala Ile Glu Tyr His Gly Glu Val Val Arg Glu Met Ser Val Ser Asp
Arg Met Cys Leu Cys Asn Met Ala Ile Glu Met Gly Ala Lys Thr Gly
Met Val Pro Pro Asp Glu Lys Thr Leu Glu Tyr Val Lys Lys Arg Ala
Gly Thr Glu Gly Arg Pro Val Glu Pro Asp Pro Asp Ala Arg Tyr Glu
Ala Glu Leu Thr Leu Asp Val Ser Asp Leu Glu Pro Gln Val Ala Lys
Pro Phe Ser Pro Asp Asn Val Val Pro Val Gly Glu Val Glu Gly Ile
Ala Ile Asp Gln Val Phe Ile Gly Ser Cys Thr Asn Gly Arg Tyr Glu
Asp Leu Lys Val Ala Ala Glu Val Leu Glu Gly Glu Glu Val His Asp
Asp Val Arg Leu Ile Val Ile Pro Ala Ser Arg Glu Val Tyr His Arg
Thr Leu Lys Asp Gly Val Leu Glu Val Leu His Glu Ala Gly Ala Leu
Ile Cys Pro Pro Asn Cys Gly Pro Cys Leu Gly Gly His Met Gly Val
Leu Ala Glu Gly Glu Arg Cys Val Ala Thr Ser Asn Arg Asn Phe Pro
Gly Arg Met Gly His Arg Glu Ser Glu Val Tyr Leu Ala Ser Pro Ala
Thr Ala Ala Ala Ser Ala Ile Glu Gly Glu Ile Thr Asp Pro Arg Pro
Tyr Leu
SEQ ID NO: 21
PRT - Methanobrevibacter smithii ATCC35061
Met Asn Ile Thr Glu Lys Ile Leu Ser Ala Lys Ala Lys Lys Glu Val
Thr Pro Gly Glu Ile Ile Glu Ile Pro Val Asp Leu Ala Met Ser His
Asp Gly Thr Ser Pro Pro Ala Ile Lys Thr Phe Glu Lys Val Ala Thr
Lys Val Trp Asp Asn Glu Lys Ile Ala Ile Val Phe Asp His Asn Val
Pro Ala Asn Thr Ile Gly Ser Ala Glu Phe Gln Lys Val Cys Arg Asp
Phe Ile Lys Lys Gln Lys Ile Thr Lys Asn Tyr Ile His Gly Asp Gly
Ile Cys His Gln Val Leu Pro Glu Lys Gly Leu Val Glu Pro Gly Lys
Val Ile Val Gly Ala Asp Ser His Thr Cys Thr Tyr Gly Ala Tyr Gly
Ala Phe Ser Thr Gly Met Gly Ala Thr Asp Leu Ala Met Val Tyr Ala
Thr Gly Lys Thr Trp Phe Met Val Pro Glu Ala Ile Lys Met Glu Val
Ser Gly Glu Leu Asn Ser Tyr Thr Ala Pro Lys Asp Ile Ile Leu Lys
Ile Ile Gly Glu Val Gly Ile Ala Gly Ala Thr Tyr Lys Thr Ala Glu
Phe Cys Gly Glu Thr Ile Glu Lys Met Gly Val Glu Gly Arg Ala Thr
Ile Cys Asn Met Ala Ile Glu Met Gly Ala Lys Asn Gly Ile Met Glu
Pro Asn Lys Glu Val Ile Gln Tyr Val Ser Gln Arg Thr Gly Lys Lys
Glu Ser Glu Leu Asn Ile Val Lys Ser Asp Glu Asp Ala Gln Tyr Ser
Glu Glu Met His Phe Asp Ile Thr Asp Met Glu Pro Gln Ile Ala Cys
Pro Asn Asp Val Asp Asn Val Lys Asp Ile Ser Lys Val Glu Gly Thr
Ala Val Asp Gln Cys Leu Ile Gly Ser Cys Thr Asn Gly Arg Leu Ser
Asp Leu Lys Asp Ala Tyr Glu Ile Leu Lys Asp Asn Glu Ile Asn Asn
Asp Thr Arg Leu Leu Ile Leu Pro Ala Ser Ala Glu Ile Tyr Lys Gln
Ala Ile His Glu Gly Tyr Ile Asp Ala Phe Ile Asp Ala Gly Ala Ile
Ile Cys Asn Pro Gly Cys Gly Pro Cys Leu Gly Gly His Met Gly Val
Leu Ser Glu Gly Glu Thr Cys Leu Ser Thr Thr Asn Arg Asn Phe Lys
Gly Arg Met Gly Asp Pro Lys Ser Ser Val Tyr Leu Ala Asn Ser Lys
Val Val Ala Ala Ser Ala Ile Glu Gly Val Ile Thr Asn Pro Lys Asp
Leu
SEQ ID NO: 22
PRT - Methanococcus vannielii SB
Met Thr Leu Ala Glu Ala Ile Leu Ser Lys Lys Leu Gly Lys Asn Val
Tyr Ala Lys Asp Ser Val Glu Ile Asp Val Asp Leu Ala Met Thr His
Asp Gly Thr Thr Pro Leu Thr Val Lys Ala Phe Glu Glu Ile Ser Asp
Arg Val Phe Asp Asn Lys Lys Ile Val Ile Val Phe Asp His Asn Ile
Pro Ala Asn Thr Ser Lys Ala Ala Asn Met Gln Ile Ile Thr Arg Asp
Phe Ile Lys Lys His Asp Ile Lys Asn Tyr Tyr Leu Asp Gly Glu Gly
Ile Cys His Gln Ile Leu Pro Glu Lys Gly His Val Lys Pro Asn Met
Val Ile Val Gly Ala Asp Ser His Thr Cys Thr His Gly Ala Phe Gly
Ala Phe Ala Thr Gly Phe Gly Ala Ser Asp Met Gly Tyr Val Tyr Ala
Thr Gly Lys Thr Trp Phe Arg Val Pro Glu Thr Ile Arg Val Asn Val
Thr Gly Lys Asn Glu Asn Ile Ser Gly Lys Asp Ile Val Leu Lys Thr
Cys Lys Glu Val Gly Arg Ser Gly Ala Thr Tyr Met Ala Leu Glu Tyr
Gly Gly Ser Ala Val Lys Ala Leu Asn Met Asp Glu Arg Met Val Leu
Cys Asn Met Ala Ile Glu Met Gly Gly Lys Val Gly Leu Ile Glu Ala
Asp His Thr Thr Tyr Asp Tyr Leu Lys Asn Ala Gly Val Ser Asn Gln
Glu Ile Ala Glu Leu Gln Arg Asn Lys Ile Ser Ile Thr Glu Asn Glu
Glu Thr Tyr Phe Lys Thr Val Glu Phe Asp Ile Thr Asp Met Glu Glu
Gln Val Ala Cys Pro His His Pro Asp Asn Val Lys Gly Ile Ser Glu
Val Leu Gly Thr Pro Ile Asp Gln Ile Phe Ile Gly Ser Cys Thr Asn
Gly His Ile Gly Asp Leu Arg Ile Ala Ala Lys Ile Leu Lys Gly Lys
Ser Ile Asn Lys Asn Thr Arg Leu Ile Val Ile Pro Ala Ser Lys Ser
Ile Leu Lys Gln Ala Leu Asn Glu Gly Leu Ile Asp Ile Phe Val Asp
Phe Gly Ala Leu Ile Cys Ala Pro Gly Cys Gly Pro Cys Leu Gly Ala
His Glu Gly Val Leu Gly Asp Gly Glu Val Cys Leu Ala Thr Thr Asn
Arg Asn Phe Lys Gly Arg Met Gly Asn Ile Asn Ser Glu Val Tyr Leu
Ser Ser Pro Ala Ile Ala Ala Lys Ser Ala Ile Lys Gly His Ile Thr
Asn Glu
SEQ ID NO: 23
PRT - Mthanococcus aeolicus Nankai 3
Met Thr Leu Ala Glu Glu Ile Leu Ser Lys Lys Val Gly Lys Lys Val
Lys Ala Gly Asp Val Val Glu Ile Asp Ile Asp Leu Ala Met Thr His
Asp Gly Thr Thr Pro Leu Ser Ala Lys Ala Phe Lys Gln Ile Thr Asp
Lys Val Trp Asp Asn Lys Lys Ile Val Ile Val Phe Asp His Asn Val
Pro Ala Asn Thr Leu Lys Ala Ala Asn Met Gln Lys Ile Thr Arg Glu
Phe Ile Lys Glu Gln Asn Ile Ile Asn His Tyr Leu Asp Gly Glu Gly
Val Cys His Gln Val Leu Pro Glu Asn Gly His Ile Gln Pro Asn Met
Val Ile Ala Gly Gly Asp Ser His Thr Cys Thr Tyr Gly Ala Phe Gly
Ala Phe Ala Thr Gly Phe Gly Ala Thr Asp Met Gly Asn Ile Tyr Ala
Thr Gly Lys Thr Trp Leu Lys Val Pro Lys Thr Ile Arg Ile Asn Val
Asn Gly Glu Asn Asp Lys Ile Thr Gly Lys Asp Ile Ile Leu Lys Ile
Cys Lys Glu Val Gly Arg Ser Gly Ala Thr Tyr Met Ala Leu Glu Tyr
Gly Gly Glu Ala Ile Lys Lys Leu Ser Met Asp Glu Arg Met Val Leu
Ser Asn Met Ala Ile Glu Met Gly Gly Lys Val Gly Leu Ile Glu Ala
Asp Glu Thr Thr Tyr Asn Tyr Leu Arg Asn Val Gly Ile Ser Glu Glu
Lys Ile Leu Glu Leu Lys Lys Asn Gln Ile Thr Ile Asp Glu Asn Asn
Ile Asp Asn Asp Asn Tyr Tyr Lys Ile Ile Asn Ile Asp Ile Thr Asp
Met Glu Glu Gln Val Ala Cys Pro His His Pro Asp Asn Val Lys Asn
Ile Ser Glu Val Lys Gly Ala Pro Ile Asn Gln Val Phe Ile Gly Ser
Cys Thr Asn Gly Arg Leu Asn Asp Leu Arg Ile Ala Ser Lys Tyr Leu
Lys Gly Lys Lys Val His Asn Asp Val Arg Leu Ile Val Ile Pro Ala
Ser Lys Ser Ile Phe Lys Gln Ala Leu Lys Glu Gly Leu Ile Asp Ile
Phe Val Asp Ala Gly Ala Leu Ile Cys Thr Pro Gly Cys Gly Pro Cys
Leu Gly Ala His Gln Gly Val Leu Gly Asp Gly Glu Val Cys Leu Ala
Thr Thr Asn Arg Asn Phe Lys Gly Arg Met Gly Asn Thr Thr Ala Glu
Ile Tyr Leu Ser Ser Pro Ala Ile Ala Ala Lys Ser Ala Ile Lys Gly
Tyr Ile Thr Asn Glu
SEQ ID NO: 24
PRT - Methanocaldococcus jannashii DSM2661
Met Ile Ile Lys Gly Arg Ala His Lys Phe Gly Asp Asp Val Asp Thr
Asp Ala Ile Ile Pro Gly Pro Tyr Leu Arg Thr Thr Asp Pro Tyr Glu
Leu Ala Ser His Cys Met Ala Gly Ile Asp Glu Asn Phe Pro Lys Lys
Val Lys Glu Gly Asp Val Ile Val Ala Gly Glu Asn Phe Gly Cys Gly
Ser Ser Arg Glu Gln Ala Val Ile Ala Ile Lys Tyr Cys Gly Ile Lys
Ala Val Ile Ala Lys Ser Phe Ala Arg Ile Phe Tyr Arg Asn Ala Ile
Asn Val Gly Leu Ile Pro Ile Ile Ala Asn Thr Asp Glu Ile Lys Asp
Gly Asp Ile Val Glu Ile Asp Leu Asp Lys Glu Glu Ile Val Ile Thr
Asn Lys Asn Lys Thr Ile Lys Cys Glu Thr Pro Lys Gly Leu Glu Arg
Glu Ile Leu Ala Ala Gly Gly Leu Val Asn Tyr Leu Lys Lys Arg Lys
Leu Ile Gln Ser Lys Lys Gly Val Lys Thr
SEQ ID NO: 25
PRT - Methanothermobacter thermoautotropicum DH
Met Glu Gly Ile Ile Arg Gly Arg Val Trp Arg Phe Gly Asp Asn Val
Asp Thr Asp Met Ile Ile Pro Gly Arg Tyr Leu Arg Thr Phe Ser Leu
Asp Glu Leu Ala Ser His Val Met Glu Gly Ala Arg Pro Glu Phe Ala
Ser Gln Val Arg Lys Gly Asp Ile Ile Val Ala Gly Arg Asn Phe Gly
Cys Gly Ser Ser Arg Glu Gln Ala Pro Val Ala Leu Lys His Ala Gly
Val Val Ala Ile Ile Ala Glu Ser Phe Ala Arg Ile Phe Tyr Arg Asn
Ala Ile Asn Ile Gly Leu Pro Val Ile Met Ala Lys Val Asp Ala Asp
Asp Gly Asp Glu Val Ser Ile Asp Leu Arg Ser Gly Gln Ile Arg Asn
Leu Thr Ala Gly Ser Glu Tyr Arg Met Lys Pro Phe Asn Asp Tyr Met
Leu Ser Ile Leu Glu Asp Gly Gly Leu Val Asn His Tyr Leu Lys Thr
Ile Asp Thr Gly Ile Ser Gly Asp Glu Gly
SEQ ID NO: 26
PRT - Methanococcus maripaludis S2
Met Lys Ile Thr Gly Lys Val His Leu Phe Gly Asp Asp Ile Asp Thr
Asp Ala Ile Ile Pro Gly Ala Tyr Leu Lys Thr Thr Asp Glu Tyr Glu
Leu Ala Ser His Cys Met Ala Gly Ile Asp Glu Asn Phe Pro Glu Arg
Val Glu Asp Gly Asp Phe Leu Val Ala Gly Glu Asn Phe Gly Cys Gly
Ser Ser Arg Glu Gln Ala Pro Ile Ala Ile Lys Tyr Cys Gly Ile Lys
Ala Ile Ile Val Glu Ser Phe Ala Arg Ile Phe Tyr Arg Asn Cys Ile
Asn Leu Gly Val Phe Pro Ile Glu Cys Lys Gly Ile Ser Lys His Val
Lys Asp Gly Asp Val Ile Glu Leu Asp Leu Glu Glu Lys Lys Val Ile
Leu Lys Asp Thr Val Leu Asp Cys Asn Leu Pro Thr Gly Thr Ala Lys
Asp Ile Met Asp Glu Gly Gly Leu Ile Asn Tyr Ala Lys Lys Gln Lys
Asn
SEQ ID NO: 27
PRT - Methanococcus maripaludis C5
Met Lys Ile Thr Gly Lys Val His Val Phe Gly Asp Asp Ile Asp Thr
Asp Ala Ile Ile Pro Gly Ala Tyr Leu Lys Thr Thr Asp Glu Tyr Glu
Leu Ala Ser His Cys Met Ala Gly Ile Asp Glu Asp Phe Pro Glu Met
Val Lys Glu Gly Asp Phe Leu Val Ala Gly Glu Asn Phe Gly Cys Gly
Ser Ser Arg Glu Gln Ala Pro Ile Ala Ile Lys Tyr Cys Gly Ile Lys
Ala Ile Ile Val Glu Ser Phe Ala Arg Ile Phe Tyr Arg Asn Cys Ile
Asn Leu Gly Val Phe Pro Ile Glu Cys Lys Gly Ile Ser Lys His Val
Lys Asp Gly Asp Leu Ile Glu Leu Asp Leu Glu Asn Lys Lys Val Ile
Leu Lys Asp Lys Val Leu Asp Cys His Ile Pro Thr Gly Thr Ala Lys
Asp Ile Met Asp Glu Gly Gly Leu Ile Asn Tyr Ala Lys Lys Gln Lys
Asn
SEQ ID NO: 28
PRT - Methanococcus maripaludis C7
Met Lys Ile Thr Gly Lys Val His Leu Phe Gly Asp Asp Val Asp Thr
Asp Ala Ile Ile Pro Gly Ala Tyr Leu Lys Thr Thr Asp Glu Tyr Glu
Leu Ala Ser His Cys Met Ala Gly Ile Asp Glu Asp Phe Pro Glu Met
Val Glu Glu Gly Asp Phe Leu Val Ala Gly Glu Asn Phe Gly Cys Gly
Ser Ser Arg Glu Gln Ala Pro Ile Ala Ile Lys Tyr Cys Gly Ile Lys
Ala Ile Ile Val Glu Ser Phe Ala Arg Ile Phe Tyr Arg Asn Cys Ile
Asn Leu Gly Val Phe Pro Ile Glu Cys Lys Gly Ile Ser Lys His Val
Lys Asp Gly Asp Ser Ile Glu Leu Asp Leu Glu Asn Lys Lys Val Ile
Leu Lys Asp Thr Val Leu Asn Cys His Leu Pro Thr Gly Thr Ala Lys
Glu Ile Met Asp Glu Gly Gly Leu Ile Asn Tyr Ala Lys Lys His Lys
Asn
SEQ ID NO: 29
PRT - Methanospaera stadtmanae DSM 3091
Met Asp Ser Met Lys Gly Lys Val Trp Thr Phe Arg Asp Cys Ile Asp
Thr Asp Val Ile Ile Ala Gly Arg Tyr Leu Arg Thr Phe Asn Pro Glu
Asp Leu Ala Ala His Val Met Glu Ala Glu Asp Pro Glu Phe Ser Ser
Lys Val Gly Lys Gly Asp Ile Ile Val Gly Gly Trp Asn Phe Gly Cys
Gly Ser Ser Arg Glu Gln Ala Pro Val Ala Ile Lys Thr Ala Gly Val
Ser Ala Val Ile Ala Lys Ser Phe Ala Arg Ile Phe Tyr Arg Asn Ala
Ile Asn Ile Gly Leu Pro Val Ile Thr Ala Asp Ile Glu Val Asp Glu
Gly Asp Ile Leu Glu Val Asn Ile Glu Asp Gly Ile Ile Ile Asn Glu
Thr Thr Lys Lys Thr Phe Lys Ile Lys Pro Phe Asp Ala Glu Met Leu
Asp Ile Leu Glu Asn Gly Gly Leu Val Asn Gln Tyr Leu Lys Asn Lys
Lys Glu Val
SEQ ID NO: 30
PRT - Methanopyrus kandleri AV19
Met Arg Asp Val Ile Arg Gly Arg Ala Trp Val Phe Gly Asp Asp Ile
Asp Thr Asp Gln Ile Ile Pro Gly Arg Tyr Leu Thr Thr Gln Asp Pro
Glu Glu Leu Ala Lys His Val Met Glu Gly Ala Asp Pro Glu Phe Pro
Glu Lys Val Arg Glu Gly Asp Val Ile Val Ala Gly Lys Asn Phe Gly
Cys Gly Ser Ser Arg Glu His Ala Pro Ile Ala Leu Lys Ala Ala Gly
Ile Ala Cys Val Val Thr Arg Ser Phe Ala Arg Ile Phe Tyr Arg Asn
Ala Ile Asn Leu Gly Leu Pro Leu Val Val Cys Pro Gly Val Asp Asp
Ala Phe Glu Asp Gly Gln Gly Ile Glu Val Asn Leu Arg Glu Gly Tyr
Val Arg Asn Leu Asp Thr Gly Glu Glu Leu Glu Ala Lys Pro Leu Pro
Asp Phe Met Met Arg Ile Leu Glu Ala Gly Gly Leu Val Glu Leu Ile
Lys Arg Glu Gly Pro Arg Ala Phe Glu Gly
SEQ ID NO: 31
PRT - Methanobrevibacter smithii ATCC35061
Met Asp Ile Ile Lys Gly Lys Thr Trp Thr Phe Gly Glu Asn Ile Asp
Thr Asp Val Ile Ile Pro Gly Arg Tyr Leu Arg Thr Phe Asn Pro Gln
Asp Leu Ala Asp His Val Leu Glu Gly Glu Arg Pro Asp Phe Thr Lys
Asn Val Lys Lys Gly Asp Ile Ile Val Ala Asp Glu Asn Phe Gly Cys
Gly Ser Ser Arg Glu Gln Ala Pro Val Ala Ile Lys Thr Ala Gly Val
Asp Ala Ile Val Ala Lys Ser Phe Ala Arg Ile Phe Tyr Arg Asn Ala
Ile Asn Ile Gly Leu Pro Val Ile Val Cys Asp Ile Gln Ala Lys Asp
Gly Asp Ile Ile Asn Ile Asp Leu Ser Lys Gly Ile Leu Thr Asn Glu
Thr Thr Gly Glu Ser Val Thr Phe Glu Pro Phe Lys Glu Phe Met Leu
Asp Ile Leu Glu Asp Asn Gly Leu Val Asn His Tyr Leu Lys Glu Lys
Gln
SEQ ID NO: 32
PRT - Methanococcus vannielii SB
Met Lys Leu Lys Gly Lys Ala His Val Phe Ser Asp Asp Val Asp Thr
Asp Ala Ile Ile Pro Gly Ala Tyr Leu Arg Thr Thr Asp Val Tyr Glu
Leu Ala Ser His Cys Met Ala Gly Ile Asp Glu Asn Phe Pro Lys Lys
Val Asn Leu Gly Asp Phe Ile Val Ala Gly Glu Asn Phe Gly Cys Gly
Ser Ser Arg Glu Gln Ala Pro Ile Ser Ile Lys Tyr Leu Gly Ile Ser
Ala Ile Ile Ala Glu Ser Phe Ala Arg Ile Phe Tyr Arg Asn Ser Ile
Asn Leu Gly Val Ile Pro Ile Glu Cys Lys Asn Ile Ser Lys His Val
Lys Thr Gly Asp Leu Ile Glu Leu Asp Leu Glu Asn Lys Lys Ile Ile
Leu Lys Asp Ile Val Leu Glu Cys Thr Val Pro Thr Gly Lys Ala Lys
Glu Ile Ile Asp Leu Gly Gly Leu Ile Asn Tyr Ala Lys Ala Gln Met
Gly
SEQ ID NO: 33
PRT - Methanococcus aeolicus Nankai 3
Met Ile Ile Lys Gly Asn Ile His Leu Phe Gly Asp Asp Ile Asp Thr
Asp Ala Ile Ile Pro Gly Ala Tyr Leu Lys Thr Thr Asp Pro Lys Glu
Leu Ala Ser His Cys Met Ala Gly Ile Asp Glu Lys Phe Ser Thr Lys
Val Lys Asp Gly Asp Ile Ile Val Ala Gly Glu Asn Phe Gly Cys Gly
Ser Ser Arg Glu Gln Ala Pro Ile Ser Ile Lys His Thr Gly Ile Lys
Ala Val Val Ala Glu Ser Phe Ala Arg Ile Phe Tyr Arg Asn Cys Ile
Asn Ile Gly Leu Ile Pro Ile Thr Cys Glu Gly Ile Asn Glu Gln Ile
Gln Asn Leu Lys Asp Gly Asp Thr Ile Glu Ile Asp Leu Gln Asn Glu
Thr Ile Lys Ile Asn Ser Met Met Leu Asn Cys Gly Ala Pro Lys Gly
Ile Glu Lys Glu Ile Leu Asp Ala Gly Gly Leu Val Gln Tyr Thr Lys
Asn Lys Leu Lys Lys
SEQ ID NO: 34
PRT - Methanocaldococcus jannashii DSM2661
Met Met Lys Val Cys Val Ile Glu Gly Asp Gly Ile Gly Lys Glu Val
Ile Pro Glu Ala Ile Lys Ile Leu Asn Glu Leu Gly Glu Phe Glu Ile
Ile Lys Gly Glu Ala Gly Leu Glu Cys Leu Lys Lys Tyr Gly Asn Ala
Leu Pro Glu Asp Thr Ile Glu Lys Ala Lys Glu Ala Asp Ile Ile Leu
Phe Gly Ala Ile Thr Ser Pro Lys Pro Gly Glu Val Gln Asn Tyr Lys
Ser Pro Ile Ile Thr Leu Arg Lys Met Phe His Leu Tyr Ala Asn Val
Arg Pro Ile Asn Asn Phe Gly Ile Gly Gln Leu Ile Gly Lys Ile Ala
Asp Tyr Glu Phe Leu Asn Ala Lys Asn Ile Asp Ile Val Ile Ile Arg
Glu Asn Thr Glu Asp Leu Tyr Val Gly Arg Glu Arg Leu Glu Asn Asp
Thr Ala Ile Ala Glu Arg Val Ile Thr Arg Lys Gly Ser Glu Arg Ile
Ile Arg Phe Ala Phe Glu Tyr Ala Ile Lys Asn Asn Arg Lys Lys Val
Ser Cys Ile His Lys Ala Asn Val Leu Arg Ile Thr Asp Gly Leu Phe
Leu Glu Val Phe Asn Glu Ile Lys Lys His Tyr Asn Ile Glu Ala Asp
Asp Tyr Leu Val Asp Ser Thr Ala Met Asn Leu Ile Lys His Pro Glu
Lys Phe Asp Val Ile Val Thr Thr Asn Met Phe Gly Asp Ile Leu Ser
Asp Glu Ala Ser Ala Leu Ile Gly Gly Leu Gly Leu Ala Pro Ser Ala
Asn Ile Gly Asp Asp Lys Ala Leu Phe Glu Pro Val His Gly Ser Ala
Pro Asp Ile Ala Gly Lys Gly Ile Ala Asn Pro Met Ala Ser Ile Leu
Ser Ile Ala Met Leu Phe Asp Tyr Ile Gly Glu Lys Glu Lys Gly Asp
Leu Ile Arg Glu Ala Val Lys Tyr Cys Leu Ile Asn Lys Lys Val Thr
Pro Asp Leu Gly Gly Asp Leu Lys Thr Lys Asp Val Gly Asp Glu Ile
Leu Asn Tyr Ile Arg Lys Lys Leu Lys Gly Tyr
SEQ ID NO: 35
PRT - Methanothermobacter thermoautotropicum DH
Met Tyr Arg Ile Thr Val Ile Pro Gly Asp Gly Ile Gly Val Glu Val
Met Glu Ala Ala Leu His Val Leu Gln Ala Leu Glu Ile Glu Phe Glu
Phe Thr His Ala Glu Ala Gly Asn Glu Cys Phe Arg Arg Cys Gly Asp
Thr Leu Pro Glu Glu Thr Leu Lys Leu Val Arg Lys Ala Asp Ala Thr
Leu Phe Gly Ala Val Thr Thr Val Pro Gly Gln Lys Ser Ala Ile Ile
Thr Leu Arg Arg Glu Leu Asp Leu Phe Ala Asn Leu Arg Pro Val Lys
Ser Leu Pro Gly Val Pro Cys Leu Tyr Pro Asp Leu Asp Phe Val Ile
Val Arg Glu Asn Thr Glu Asp Leu Tyr Val Gly Asp Glu Glu Tyr Thr
Pro Glu Gly Ala Val Ala Lys Arg Ile Ile Thr Arg Thr Ala Ser Arg
Arg Ile Ser Gln Phe Ala Phe Gln Tyr Ala Gln Lys Glu Gly Met Gln
Lys Val Thr Ala Val His Lys Ala Asn Val Leu Lys Lys Thr Asp Gly
Ile Phe Arg Asp Glu Phe Tyr Lys Val Ala Ser Glu Tyr Pro Gln Met
Glu Ala Asn Asp Tyr Tyr Val Asp Ala Thr Ala Met Tyr Leu Ile Thr
Gln Pro Gln Glu Phe Gln Thr Ile Val Thr Thr Asn Leu Phe Gly Asp
Ile Leu Ser Asp Glu Ala Ala Gly Leu Ile Gly Gly Leu Gly Leu Ala
Pro Ser Ala Asn Ile Gly Glu Lys Asn Ala Leu Phe Glu Pro Val His
Gly Ser Ala Pro Gln Ile Ala Gly Lys Asn Ile Ala Asn Pro Thr Ala
Met Ile Leu Thr Thr Thr Leu Met Leu Lys His Leu Asn Lys Lys Gln
Glu Ala Gln Lys Ile Glu Lys Ala Leu Gln Lys Thr Leu Met Arg Gly
Ile Met Thr Pro Asp Leu Gly Gly Thr Ala Ser Thr Met Glu Met Ala
Glu Ala Ile Lys Glu Glu Ile Val Lys Gly Glu
SEQ ID NO: 36
PRT - Methanococcus maripaludis S2
Met Arg Asn Thr Pro Lys Ile Cys Val Ile Asn Gly Asp Gly Ile Gly
Asn Glu Val Val Pro Glu Thr Val Arg Val Leu Asn Glu Leu Gly Asp
Phe Glu Phe Ile His Ala His Ala Gly Tyr Glu Cys Phe Lys Arg Cys
Gly Asp Ala Ile Pro Glu Asn Thr Ile Glu Ile Ala Lys Glu Ser Asp
Cys Ile Leu Phe Gly Ser Val Thr Thr Pro Lys Pro Thr Glu Leu Lys
Asn Lys Ser Tyr Arg Ser Pro Ile Leu Thr Leu Arg Lys Glu Leu Asp
Leu Tyr Ala Asn Ile Arg Pro Thr Tyr Asn Phe Asp Asn Leu Asp Phe
Val Ile Ile Arg Glu Asn Thr Glu Gly Leu Tyr Val Lys Lys Glu Tyr
Tyr Asp Glu Lys Asn Glu Val Ala Ile Ala Glu Arg Ile Ile Ser Lys
Phe Gly Ser Ser Arg Ile Val Lys Phe Ala Phe Asp Tyr Ala Val Gln
Asn Asn Arg Lys Lys Val Ser Cys Ile His Lys Ala Asn Val Leu Arg
Val Thr Asp Gly Leu Phe Leu Glu Val Phe Glu Glu Met Ser Lys His
Tyr Glu Lys Leu Gly Ile Lys Ser Asp Asp Tyr Leu Ile Asp Ala Thr
Ala Met Tyr Leu Ile Arg Asn Pro Gln Met Phe Asp Val Leu Val Thr
Thr Asn Leu Phe Gly Asp Ile Leu Ser Asp Glu Ala Ala Gly Leu Ile
Gly Gly Leu Gly Met Ser Pro Ser Ala Asn Ile Gly Asp Lys Asn Gly
Leu Phe Glu Pro Val His Gly Ser Ala Pro Asp Ile Ala Gly Lys Gly
Ile Ser Asn Pro Ile Ala Thr Ile Leu Ser Ala Ala Met Met Leu Asp
His Leu Lys Met Asn Lys Glu Ala Glu Tyr Ile Arg Lys Ala Val Lys
Lys Thr Val Glu Cys Lys Tyr Leu Thr Pro Asp Leu Gly Gly Asn Leu
Lys Thr Phe Glu Val Thr Glu Lys Ile Ile Glu Ser Ile Arg Ser Gln
Met Ile Gln
SEQ ID NO: 37
PRT - Methanococcus maripaludis C5
Met Arg Asn Thr Pro Lys Ile Cys Val Ile Asn Gly Asp Gly Ile Gly
Asn Glu Val Ile Pro Glu Thr Val Arg Val Leu Asn Glu Ile Gly Asp
Phe Glu Phe Ile Glu Thr His Ala Gly Tyr Glu Cys Phe Lys Arg Cys
Gly Asp Ala Ile Pro Glu Lys Thr Ile Glu Ile Ala Lys Glu Ser Asp
Ser Ile Leu Phe Gly Ser Val Thr Thr Pro Lys Pro Thr Glu Leu Lys
Asn Lys Pro Tyr Arg Ser Pro Ile Leu Thr Leu Arg Lys Glu Leu Asp
Leu Tyr Ala Asn Ile Arg Pro Thr Phe Asn Phe Lys Asn Leu Asp Phe
Val Ile Ile Arg Glu Asn Thr Glu Gly Leu Tyr Val Lys Lys Glu Tyr
Tyr Asp Glu Lys Asn Glu Val Ala Thr Ala Glu Arg Ile Ile Ser Lys
Phe Gly Ser Ser Arg Ile Val Lys Phe Ala Phe Asp Tyr Ala Leu Gln
Asn Asn Arg Lys Lys Val Ser Cys Ile His Lys Ala Asn Val Leu Arg
Ile Thr Asp Gly Leu Phe Leu Gly Val Phe Glu Glu Ile Ser Lys Lys
Tyr Glu Lys Leu Gly Ile Val Ser Asp Asp Tyr Leu Ile Asp Ala Thr
Ala Met Tyr Leu Ile Arg Asn Pro Gln Met Phe Asp Val Met Val Thr
Thr Asn Leu Phe Gly Asp Ile Leu Ser Asp Glu Ala Ala Gly Leu Ile
Gly Gly Leu Gly Met Ser Pro Ser Ala Asn Ile Gly Asp Lys Asn Gly
Leu Phe Glu Pro Val His Gly Ser Ala Pro Asp Ile Ala Gly Lys Gly
Ile Ser Asn Pro Ile Ala Thr Ile Leu Ser Ala Ala Met Met Leu Asp
His Leu Lys Ile Asn Lys Glu Ala Glu Tyr Ile Arg Asn Ala Val Lys
Lys Thr Val Glu Cys Lys Tyr Leu Thr Pro Asp Leu Gly Gly His Leu
Lys Thr Ser Glu Val Thr Glu Lys Ile Ile Glu Ser Ile Lys Ser Gln
Met Ile Gln
SEQ ID NO: 38
PRT - Methanococcus maripaludis C7
Met Arg Asn Thr Pro Lys Ile Cys Val Ile Asn Gly Asp Gly Ile Gly
Asn Glu Val Ile Pro Glu Thr Val Arg Val Leu Ser Glu Ile Gly Asp
Phe Glu Phe Ile Glu Thr His Ala Gly Tyr Glu Cys Phe Lys Arg Cys
Gly Asp Ala Ile Pro Glu Lys Thr Ile Glu Ile Ala Lys Glu Ser Asp
Ser Ile Leu Phe Gly Ser Val Thr Thr Pro Lys Pro Thr Glu Leu Lys
Asn Lys Pro Tyr Arg Ser Pro Ile Leu Thr Leu Arg Lys Glu Leu Asp
Leu Tyr Ala Asn Ile Arg Pro Thr Phe Asn Phe Lys Asp Leu Asp Phe
Val Ile Ile Arg Glu Asn Thr Glu Gly Leu Tyr Val Lys Lys Glu Tyr
Tyr Asp Glu Lys Asn Glu Val Ala Ile Ala Glu Arg Val Ile Ser Lys
Phe Gly Ser Ser Arg Ile Val Lys Tyr Ala Phe Asp Tyr Ala Leu Gln
Asn Asn Arg Lys Lys Val Ser Cys Ile His Lys Ala Asn Val Leu Arg
Ile Thr Asp Gly Leu Phe Leu Glu Val Phe Glu Glu Ile Ser Lys Lys
Tyr Glu Lys Leu Gly Ile Ala Ser Asp Asp Tyr Leu Ile Asp Ala Thr
Ala Met Tyr Leu Ile Arg Asn Pro Gln Met Phe Asp Val Met Val Thr
Thr Asn Leu Phe Gly Asp Ile Leu Ser Asp Glu Ala Ala Gly Leu Ile
Gly Gly Leu Gly Met Ser Pro Ser Ala Asn Ile Gly Asp Lys Asn Gly
Leu Phe Glu Pro Val His Gly Ser Ala Pro Asp Ile Ala Gly Lys Gly
Ile Ser Asn Pro Ile Ala Ser Ile Leu Ser Ala Ala Met Met Leu Asp
His Leu Asn Met Asn Lys Glu Ala Glu Cys Ile Arg Asn Ala Val Lys
Lys Ala Val Glu Cys Lys Tyr Leu Thr Pro Asp Leu Gly Gly Asn Leu
Lys Thr Ser Glu Val Thr Asp Lys Ile Ile Glu Ser Ile Lys Ser Gln
Met Val Gln
SEQ ID NO: 39
PRT - Methanospaera stadtmanae DSM 3091
Met Tyr Lys Ile Thr Val Ile Pro Gly Asp Gly Ile Gly Gln Glu Val
Met Gln Pro Thr Ile Asp Ile Leu Glu Thr Leu Asn Ser Lys Phe Glu
Phe Ile Pro Lys Glu Ala Gly Lys Glu Cys Tyr Gln Lys Tyr Asp Thr
Asn Leu Pro Glu Glu Thr Ile Val Gln Cys Arg Glu Ser Asp Ser Thr
Leu Phe Gly Ala Val Thr Ser Ile Pro Gln Gln Lys Ser Ala Ile Val
Thr Leu Arg Lys Glu Leu Asp Leu Tyr Val Asn Gln Arg Pro Ile His
Ser Tyr Thr Asn Pro Asp Ile Asp Phe Thr Ile Ile Arg Glu Asn Ser
Glu Gly Leu Tyr Ser His Ile Glu Glu Ser Thr Gly Asp Glu Ala Ile
Ala Ile Arg Lys Ile Thr Tyr Lys Ala Ser Glu Arg Ile Ile Asn Tyr
Ala Phe Asn Tyr Ala Leu Lys Thr Glu Lys Ser Lys Val Thr Ala Ser
His Lys Ala Asn Val Leu Pro Val Thr Asp Gly Ile Phe Lys Asn Thr
Phe Tyr Lys Val Ala Ser Asn Tyr Pro Thr Ile Lys Ser Asn Asp Tyr
Tyr Ile Asp Ala Met Ala Met Tyr Leu Ile Thr Asn Pro Ala Gln Phe
Asp Ile Ile Val Thr Thr Asn Leu Phe Gly Asp Ile Leu Ser Asp Glu
Gly Gly Gly Leu Val Gly Thr Leu Gly Leu Ile Pro Ser Ala Asn Ile
Gly Asp Lys Thr Gly Leu Phe Glu Pro Val His Gly Ser Ala Pro Asp
Ile Ala Gly Leu Asn Lys Ala Asn Pro Ile Ala Met Ile Leu Ser Ser
Cys Leu Met Leu Glu Tyr Leu Gly Leu Tyr Asp Asp Ala Lys Arg Ile
Gln Asn Ala Val Glu Glu Thr Ile Ser Glu Ser Lys Val Lys Thr Pro
Asp Met Gly Gly His Asn Asn Thr Gln Asp Val Ala Asn Asn Ile Leu
His Arg Leu
SEQ ID NO: 40
PRT - Methanopyrus kandleri AV19
Met Ala Tyr Lys Ile Ala Val Ile Pro Gly Asp Gly Ile Gly Pro Glu
Val Ile Glu Ala Ala Leu His Val Ile Glu Pro Leu Ile Asp Ala Glu
Phe Val Glu Gly Glu Ala Gly Asp Glu Cys Ala Glu Lys His Gly Asp
Pro Leu Pro Glu Asp Thr Leu Glu Leu Cys His Glu Ala Asp Ala Ile
Leu Phe Gly Ala Ala Gly Glu Thr Ala Ala Asp Val Ile Val Arg Leu
Arg Gln Glu Leu Asp Leu Tyr Ala Asn Ile Arg Pro Val Arg Gly Phe
Pro Gly Leu Arg Glu Leu Thr Gly Glu Pro Tyr Val Arg Asp Asp Val
Asp Phe Val Ile Val Arg Glu Asn Thr Glu Gly Leu Tyr Ser Gly Ile
Glu Gly Arg Phe Arg Asp Thr Ala Tyr Thr Leu Arg Ile Ile Thr Glu
Glu Gly Thr Arg Arg Ile Ala Glu Val Ala Cys Asp Leu Ala Glu Glu
Arg Gly Ser Asn Thr Val Thr Cys Val His Lys Ala Asn Val Met Arg
Glu Thr Cys Gly Leu Phe Arg Glu Val Cys Lys Glu Val Val Glu Ser
Arg Gly Leu Glu Phe Glu Glu Tyr Tyr Val Asp Ala Ala Ala Met Phe
Met Ile Thr Glu Pro Glu Arg Phe Asp Val Val Val Thr Pro Asn Met
Phe Gly Asp Ile Leu Ser Asp Glu Ala Ala Ala Leu Val Gly Gly Leu
Gly Leu Ala Pro Ser Gly Asn Val Gly Asp Arg His Gly Leu Phe Glu
Pro Val His Gly Ser Ala Pro Asp Ile Ala Gly Lys Gly Ile Ala Asn
Pro Phe Ala Thr Ile Leu Ser Ala Val Met Met Leu Glu Trp Leu Gly
Glu Asp Glu Ala Ala Glu Ala Val Arg Glu Ala Val Gly Glu Ala Ile
Arg Glu Gly Val Val Thr Pro Asp Leu Gly Gly Asp Lys Lys Thr Met
Glu Val Ala Glu Phe Val Arg Glu Ala Ala Leu Asn Arg Val Gln
SEQ ID NO: 41
PRT - Methanobrevibacter smithii ATCC35061
Met Ser Thr Ser Asn Lys Lys Asp Asn Lys Tyr Gln Ile Ala Val Ile
Pro Gly Asp Gly Ile Gly Lys Glu Val Met Glu Ala Thr Ile Ser Val
Leu Asp Glu Leu Asp Val Asp Phe Asp Tyr Ile Tyr Gly Ile Ala Gly
Asp Glu Cys Asn Glu Glu His Gly Thr Pro Leu Pro Gln Glu Thr Ile
Asp Ile Val Arg Asp Ser Asp Ala Cys Leu Phe Gly Ala Ala Gly Glu
Thr Ala Ala Asp Val Ile Val Lys Ile Arg Gln Glu Met Lys Met Phe
Ala Asn Leu Arg Pro Val Lys Ser Tyr Pro Asn Thr Lys Ser Leu Phe
Glu Asn Val Asp Phe Met Ile Val Arg Glu Asn Thr Glu Gly Leu Tyr
Ile Ala Asp Gln Glu Glu Glu Thr Glu Asp Gly Ala Ile Ala Lys Arg
Val Ile Thr Arg Glu Ala Glu Glu Arg Ile Ile Asp Tyr Ala Phe Gln
Tyr Ala Lys Asp Asn Asn Arg Thr Lys Val Thr Ala Val His Lys Ala
Asn Val Leu Lys Lys Thr Asp Gly Leu Phe Lys Lys Ile Phe Tyr Glu
Val Gly Glu Lys Tyr Pro Asp Ile Asp Thr Glu Asp Phe Tyr Val Asp
Ala Thr Ala Met Tyr Leu Val Thr Gln Pro Gln Glu Phe Gln Val Val
Val Thr Thr Asn Leu Phe Gly Asp Ile Leu Ser Asp Glu Gly Ala Gly
Leu Val Gly Gly Leu Gly Leu Ile Pro Ser Ala Asn Ile Gly Ala Asp
Gly Ala Leu Phe Glu Pro Val His Gly Ser Ala Pro Asp Ile Ala Gly
Gln Gln Lys Ala Asn Pro Ile Ala Met Met Leu Ser Ala Ile Met Met
Leu Arg Tyr Leu Gly Glu Asn Asp Ala Ala Asp Lys Phe Asp Ala Ala
Ile Leu Lys Val Leu Ser Glu Gly Lys Thr Leu Thr Gly Asp Leu Gly
Gly Ser Ala Thr Thr Met Glu Val Ala Gln Ala Val Lys Asn Ala Leu
SEQ ID NO: 42
PRT - Methanococcus vannielii SB
Met Gly Tyr Met Pro Lys Ile Cys Val Ile Thr Gly Asp Gly Ile Gly
Lys Glu Val Val Pro Glu Thr Leu Arg Val Leu Asn Glu Val His Asp
Phe Glu Tyr Ile Glu Ala His Ala Gly Tyr Glu Cys Phe Lys Arg Cys
Gly Glu Ser Ile Pro Glu Ser Thr Ile Gln Thr Ala Lys Asn Ser Asp
Ser Ile Leu Phe Gly Ser Val Thr Thr Pro Lys Pro Thr Glu Leu Lys
Asn Lys Pro Tyr Arg Ser Pro Ile Leu Thr Leu Arg Gln Glu Leu Asp
Leu Tyr Ala Asn Ile Arg Pro Thr Tyr Asn Phe Lys Asp Leu Asp Phe
Val Ile Ile Arg Glu Asn Thr Glu Cys Leu Tyr Val Lys Arg Glu Tyr
Tyr Asp Glu Ile Asn Glu Val Ala Ile Ala Glu Arg Ile Ile Ser Lys
Lys Gly Ser Glu Arg Ile Ile Lys Phe Ala Phe Glu Tyr Ala Arg Leu
Asn Asn Arg Lys Lys Val Ser Cys Ile His Lys Ala Asn Val Leu Arg
Val Thr Asp Gly Leu Phe Leu Glu Ile Phe Glu Lys Ile Ala Lys Leu
Tyr Glu Asn Phe Gly Ile Ser Ser Asn Asp Tyr Leu Ile Asp Ala Thr
Ala Met Tyr Leu Ile Lys Asn Pro Tyr Met Phe Asp Val Met Val Thr
Thr Asn Leu Phe Gly Asp Ile Leu Ser Asp Glu Ala Ala Gly Leu Ile
Gly Gly Leu Gly Met Ser Pro Ser Ala Asn Ile Gly Asp Asn Leu Gly
Leu Phe Glu Pro Val His Gly Ser Ala Pro Asp Ile Ala Gly Lys Gly
Ile Ser Asn Pro Ile Ala Thr Ile Leu Ser Ala Ser Met Met Leu Asp
His Leu Lys Met Asn Lys Lys Ala Glu Ile Ile Arg Asn Ala Val Lys
Lys Thr Ile Asn Asn Gly Tyr Leu Thr Pro Asp Leu Gly Gly Ser Leu
Lys Thr Ser Glu Val Val Asn Lys Val Ile Glu Phe Ile Arg Asp Glu
Ile
SEQ ID NO: 43
PRT - Methanococcus aeolicus Nankai 3
Met Lys Ile Pro Lys Ile Cys Val Ile Glu Gly Asp Gly Ile Gly Lys
Glu Val Ile Pro Glu Thr Val Arg Ile Leu Lys Glu Ile Gly Asp Phe
Glu Phe Ile Tyr Glu His Ala Gly Tyr Glu Cys Phe Lys Arg Cys Gly
Asp Ala Ile Pro Glu Lys Thr Leu Lys Thr Ala Lys Glu Cys Asp Ala
Ile Leu Phe Gly Ala Val Ser Thr Pro Lys Leu Asp Glu Thr Glu Arg
Lys Pro Tyr Lys Ser Pro Ile Leu Thr Leu Arg Lys Glu Leu Asp Leu
Tyr Ala Asn Val Arg Pro Ile His Lys Leu Asp Asn Ser Asp Ser Ser
Asn Asn Ile Asp Phe Ile Ile Ile Arg Glu Asn Thr Glu Gly Leu Tyr
Ser Gly Val Glu Tyr Tyr Asp Glu Glu Lys Glu Leu Ala Ile Ser Glu
Arg His Ile Ser Lys Lys Gly Ser Lys Arg Ile Ile Lys Phe Ala Phe
Glu Tyr Ala Val Lys His His Arg Lys Lys Val Ser Cys Ile His Lys
Ser Asn Ile Leu Arg Ile Thr Asp Gly Leu Phe Leu Asn Ile Phe Asn
Glu Phe Lys Glu Lys Tyr Lys Asn Glu Tyr Asn Ile Glu Gly Asn Asp
Tyr Leu Val Asp Ala Thr Ala Met Tyr Ile Leu Lys Ser Pro Gln Met
Phe Asp Val Ile Val Thr Thr Asn Leu Phe Gly Asp Ile Leu Ser Asp
Glu Ala Ser Gly Leu Leu Gly Gly Leu Gly Leu Ala Pro Ser Ala Asn
Ile Gly Asp Asn Tyr Gly Leu Phe Glu Pro Val His Gly Ser Ala Pro
Asp Ile Ala Gly Lys Gly Val Ala Asn Pro Ile Ala Ala Val Leu Ser
Ala Ser Met Met Leu Tyr Tyr Leu Asp Met Lys Glu Lys Ser Arg Leu
Leu Lys Asp Ala Val Lys Gln Val Leu Ala His Lys Asp Ile Thr Pro
Asp Leu Gly Gly Asn Leu Lys Thr Lys Glu Val Ser Asp Lys Ile Ile
Glu Glu Leu Arg Lys Ile Ser
SEQ ID NO: 44
PRT - Saccharomyces cerevisiae
Met Ser Glu Asn Asn Glu Phe Gln Ser Val Thr Glu Ser Thr Thr Ala
Pro Thr Thr Ser Asn Pro Tyr Gly Pro Asn Pro Ala Asp Tyr Leu Ser
Asn Val Lys Asn Phe Gln Leu Ile Asp Ser Thr Leu Arg Glu Gly Glu
Gln Phe Ala Asn Ala Phe Phe Asp Thr Glu Lys Lys Ile Glu Ile Ala
Arg Ala Leu Asp Asp Phe Gly Val Asp Tyr Ile Glu Leu Thr Ser Pro
Val Ala Ser Glu Gln Ser Arg Lys Asp Cys Glu Ala Ile Cys Lys Leu
Gly Leu Lys Ala Lys Ile Leu Thr His Ile Arg Cys His Met Asp Asp
Ala Arg Val Ala Val Glu Thr Gly Val Asp Gly Val Asp Val Val Ile
Gly Thr Ser Lys Phe Leu Arg Gln Tyr Ser His Gly Lys Asp Met Asn
Tyr Ile Ala Lys Ser Ala Val Glu Val Ile Glu Phe Val Lys Ser Lys
Gly Ile Glu Ile Arg Phe Ser Ser Glu Asp Ser Phe Arg Ser Asp Leu
Val Asp Leu Leu Asn Ile Tyr Lys Thr Val Asp Lys Ile Gly Val Asn
Arg Val Gly Ile Ala Asp Thr Val Gly Cys Ala Asn Pro Arg Gln Val
Tyr Glu Leu Ile Arg Thr Leu Lys Ser Val Val Ser Cys Asp Ile Glu
Cys His Phe His Asn Asp Thr Gly Cys Ala Ile Ala Asn Ala Tyr Thr
Ala Leu Glu Gly Gly Ala Arg Leu Ile Asp Val Ser Val Leu Gly Ile
Gly Glu Arg Asn Gly Ile Thr Pro Leu Gly Gly Leu Met Ala Arg Met
Ile Val Ala Ala Pro Asp Tyr Val Arg Ser Lys Tyr Lys Leu His Lys
Ile Arg Asp Ile Glu Asn Leu Val Ala Asp Ala Val Glu Val Asn Ile
Pro Phe Asn Asn Pro Ile Thr Gly Phe Cys Ala Phe Thr His Lys Ala
Gly Ile His Ala Lys Ala Ile Leu Ala Asn Pro Ser Thr Tyr Glu Ile
Leu Asp Pro His Asp Phe Gly Met Lys Arg Tyr Ile His Phe Ala Asn
Arg Leu Thr Gly Trp Asn Ala Ile Lys Ser Arg Val Asp Gln Leu Asn
Leu Asn Leu Thr Asp Asp Gln Ile Lys Glu Val Thr Ala Lys Ile Lys
Lys Leu Gly Asp Val Arg Pro Leu Asn Ile Asp Asp Val Asp Ser Ile
Ile Lys Asp Phe His Ala Glu Leu Ser Thr Pro Leu Leu Lys Pro Val
Asn Lys Gly Thr Asp Asp Asp Asn Ile Asp Ile Ser Asn Gly His Val
Ser Lys Lys Ala Lys Val Thr Lys
SEQ ID NO: 45
PRT - Saccharomyces cerevisiae
Met Thr Ala Ala Lys Pro Asn Pro Tyr Ala Ala Lys Pro Gly Asp Tyr
Leu Ser Asn Val Asn Asn Phe Gln Leu Ile Asp Ser Thr Leu Arg Glu
Gly Glu Gln Phe Ala Asn Ala Phe Phe Asp Thr Glu Lys Lys Ile Glu
Ile Ala Arg Ala Leu Asp Asp Phe Gly Val Asp Tyr Ile Glu Leu Thr
Ser Pro Val Ala Ser Glu Gln Ser Arg Lys Asp Cys Glu Ala Ile Cys
Lys Leu Gly Leu Lys Ala Lys Ile Leu Thr His Ile Arg Cys His Met
Asp Asp Ala Lys Val Ala Val Glu Thr Gly Val Asp Gly Val Asp Val
Val Ile Gly Thr Ser Lys Phe Leu Arg Gln Tyr Ser His Gly Lys Asp
Met Asn Tyr Ile Ala Lys Ser Ala Val Glu Val Ile Glu Phe Val Lys
Ser Lys Gly Ile Glu Ile Arg Phe Ser Ser Glu Asp Ser Phe Arg Ser
Asp Leu Val Asp Leu Leu Asn Ile Tyr Lys Thr Val Asp Lys Ile Gly
Val Asn Arg Val Gly Ile Ala Asp Thr Val Gly Cys Ala Asn Pro Arg
Gln Val Tyr Glu Leu Ile Arg Thr Leu Lys Ser Val Val Ser Cys Asp
Ile Glu Cys His Phe His Asn Asp Thr Gly Cys Ala Ile Ala Asn Ala
Tyr Thr Ala Leu Glu Gly Gly Ala Arg Leu Ile Asp Val Ser Val Leu
Gly Ile Gly Glu Arg Asn Gly Ile Thr Pro Leu Gly Gly Leu Met Ala
Arg Met Ile Val Ala Ala Pro Asp Tyr Val Lys Ser Lys Tyr Lys Leu
His Lys Ile Arg Asp Ile Glu Asn Leu Val Ala Asp Ala Val Glu Val
Asn Ile Pro Phe Asn Asn Pro Ile Thr Gly Phe Cys Ala Phe Thr His
Lys Ala Gly Ile His Ala Lys Ala Ile Leu Ala Asn Pro Ser Thr Tyr
Glu Ile Leu Asp Pro His Asp Phe Gly Met Lys Arg Tyr Ile His Phe
Ala Asn Arg Leu Thr Gly Trp Asn Ala Ile Lys Ala Arg Val Asp Gln
Leu Asn Leu Asn Leu Thr Asp Asp Gln Ile Lys Glu Val Thr Ala Lys
Ile Lys Lys Leu Gly Asp Val Arg Ser Leu Asn Ile Asp Asp Val Asp
Ser Ile Ile Lys Asn Phe His Ala Glu Val Ser Thr Pro Gln Val Leu
Ser Ala Lys Lys Asn Lys Lys Asn Asp Ser Asp Val Pro Glu Leu Ala
Thr Ile Pro Ala Ala Lys Arg Thr Lys Pro Ser Ala
SEQ ID NO: 46
PRT - Kluyveromyces lactis
Met Ser Val Asn Ser Asn Pro Tyr Ala Pro Ser Pro Asn Asp Leu Leu
Ser Asn Val Cys Asn Phe Gln Leu Ile Glu Ser Thr Leu Arg Glu Gly
Glu Gln Phe Ala Ser Ala Phe Phe Ser Thr Glu Lys Lys Ile Glu Ile
Ala Lys Ala Leu Asp Asp Phe Gly Val Asp Tyr Ile Glu Leu Thr Ser
Pro Val Ala Ser Glu Gln Ser Arg Ser Asp Cys Glu Ala Ile Cys Lys
Leu Gly Leu Lys Ala Lys Ile Leu Thr His Ile Arg Cys His Met Asp
Asp Ala Arg Val Ala Val Glu Thr Gly Val Asp Gly Val Asp Val Val
Ile Gly Thr Ser Lys Phe Leu Arg Glu Tyr Ser His Gly Lys Asp Met
Asn Tyr Ile Ala Lys Ser Ala Ile Glu Val Ile Glu Phe Val Lys Ser
Lys Gly Leu Glu Ile Arg Phe Ser Ser Glu Asp Ser Phe Arg Ser Asp
Ile Val Asp Leu Leu Asn Ile Tyr Lys Thr Val Asp Lys Ile Gly Val
Asn Arg Val Gly Ile Ala Asp Thr Val Gly Cys Ala Asn Pro Arg Gln
Val Tyr Glu Leu Val Arg Thr Leu Lys Ser Val Val Ser Cys Asp Ile
Glu Cys His Phe His Asp Asp Thr Gly Cys Ala Ile Gly Asn Ser Tyr
Ser Ala Leu Glu Ala Gly Ala Arg Leu Ile Asp Val Ser Val Leu Gly
Ile Gly Glu Arg Asn Gly Ile Thr Ser Leu Gly Gly Leu Met Ala Arg
Met Ile Val Ser Ala Pro Glu Tyr Val Lys Ser Lys Tyr Lys Leu His
Lys Leu Arg Asp Leu Glu Asn Leu Val Ala Asp Ala Val Ser Val Asn
Val Pro Phe Asn Asn Pro Ile Thr Gly Phe Cys Ala Phe Thr His Lys
Ala Gly Ile His Ala Lys Ala Ile Leu Ala Asn Pro Ser Thr Tyr Glu
Ile Leu Asn Pro Glu Asp Phe Gly Met Lys Arg Tyr Ile His Phe Ala
Asn Arg Leu Thr Gly Trp Asn Ala Ile Lys Ser Arg Val Glu Gln Leu
Asn Leu His Leu Ser Asp Asp Gln Ile Lys Glu Val Thr Ser Lys Ile
Lys Gln Ile Gly Asp Val Arg Gln Leu Ser Ile Glu Asp Val Asp Thr
Ile Ile Lys Asp Tyr His Ser Glu Leu
SEQ ID NO: 47
PRT - Phanerochaete chrysosporium
misc_feature: Xaa can be any naturally occurring amino acid
Leu Ser Ile Leu Val Ala Ile Gln Lys Leu Glu Pro Cys Cys Lys Met
Cys Pro His Ala Asn Gly Asp Ser Thr Pro Asn Asp Pro Ser Gln Met
Val Pro Val Asp Leu Ser Asn Gly Thr Ser His Gln Ala Ser Val Gln
Ser Asn Ser Asn Gly His Ala Ala Thr Asn Gly Ala Ala Xaa Asn Pro
Tyr Ala Pro Arg Ala Ser Asp Phe Leu Ser Asn Val Ser Asn Phe Lys
Ile Ile Glu Ser Thr Leu Arg Glu Gly Glu Gln Phe Ala Asn Ala Phe
Phe Asp Thr Lys Thr Lys Ile Ala Ile Ala Lys Ala Leu Asp Ala Phe
Gly Val Glu Tyr Ile Glu Leu Thr Ser Pro Ala Ala Ser Glu Gln Ser
Arg Arg Asp Cys Glu Ala Ile Cys Lys Leu Gly Leu Lys Ala Lys Ile
Leu Thr His Ile Arg Cys His Met Asp Asp Ala Arg Ile Ala Val Glu
Thr Gly Val Asp Gly Val Asp Val Val Ile Gly Thr Ser Ser Phe Leu
Arg Glu Phe Ser His Gly Lys Asp Met Ala Tyr Ile Thr Lys Thr Ala
Ile Glu Val Ile Glu Phe Val Lys Ser Lys Gly Ile Glu Val Arg Phe
Ser Ser Glu Asp Ser Phe Arg Ser Asp Leu Val Asp Leu Leu Ser Ile
Tyr Gln Thr Val Asp Lys Ile Gly Val Asn Arg Val Gly Ile Ala Asp
Thr Val Gly Cys Ala Asn Pro Arg Gln Val Tyr Asp Leu Val Arg Thr
Leu Arg Gly Val Val Lys Cys Asp Ile Glu Ile His Leu His Asn Asp
Thr Gly Met Ala Ile Ala Asn Ala Tyr Thr Ala Leu Glu Ala Gly Ala
Thr His Ile Asp Thr Ser Val Leu Gly Ile Gly Glu Arg Val Gly Ile
Thr Pro Leu Gly Gly Leu Val Ala Cys Leu Tyr Ala Ala Asn Pro Glu
Tyr Val Lys Ser Lys Tyr Asn Leu Pro Met Leu Arg Glu Ile Glu Asn
Leu Val Ala Glu Ala Val Glu Val Asn Ile Pro Phe Met Asn Pro Ile
Thr Gly Tyr Cys Ala Phe Thr His Lys Ala Gly Ile His Ala Lys Ala
Ile Leu Asn Asn Pro Ser Thr Tyr Glu Ile Leu Lys Pro Glu Asp Phe
Gly Leu Thr Arg Tyr Val Ser Ile Gly His Arg Leu Thr Gly Trp Asn
Ala Val Lys Ser Arg Val Glu Gln Leu Gly Leu Lys Leu Thr Asp Glu
Glu Ile Lys Asp Val Thr Ala Lys Ile Lys Glu Leu Ala Asp Val Arg
Thr Gln Ser Met Asp Asp Val Asp Thr Leu Leu Arg Val Tyr His Ser
Gly Ile Gln Ser Gly Glu Leu Ala Ala Gly Gln Arg Glu Ala Leu Asp
Arg Leu Leu Arg Lys His Arg Glu Gly Thr Met Ser Arg Glu Pro Ser
Val Ser Arg Pro Ser Thr Pro Thr Gln Ala
SEQ ID NO: 48
PRT - Kluyveromyces lactis
Met Ser Ser Asn Gln Asp Phe Gln Pro Val Thr Glu Ser Ala Ser Ser
Val Thr Lys Phe Gln Gln Asn Pro Tyr Gly Pro Asn Pro Ala Asp Tyr
Leu Ser Asn Val Asn Asn Tyr Gln Leu Ile Asp Ser Thr Leu Arg Glu
Gly Glu Gln Phe Ala Asn Ala Phe Phe Asp Thr Glu Lys Lys Ile Glu
Ile Ala Lys Ala Leu Asp Asp Phe Gly Val Asp Tyr Ile Glu Leu Thr
Ser Pro Val Ala Ser Glu Gln Ser Arg Arg Asp Cys Glu Ala Ile Cys
Lys Leu Gly Leu Lys Ala Lys Ile Leu Thr His Ile Arg Cys His Met
Asp Asp Ala Arg Val Ala Val Glu Thr Gly Val Asp Gly Val Asp Val
Val Ile Gly Thr Ser Lys Phe Leu Arg Gln Tyr Ser His Gly Lys Asp
Met Asn Tyr Ile Ala Lys Ser Ala Ile Glu Val Ile Glu Phe Val Lys
Ser Lys Gly Ile Glu Ile Arg Phe Ser Ser Glu Asp Ser Phe Arg Ser
Asp Leu Val Asp Leu Leu Asn Ile Tyr Lys Thr Val Asp Lys Ile Gly
Val Asn Arg Val Gly Ile Ala Asp Thr Val Gly Cys Ala Asn Pro Arg
Gln Val Tyr Glu Leu Val Arg Thr Leu Lys Ser Val Val Ser Cys Asp
Ile Glu Cys His Phe His Asn Asp Thr Gly Cys Ala Ile Ala Asn Ala
Tyr Thr Ala Leu Glu Gly Gly Ala Arg Leu Ile Asp Val Ala Val Leu
Gly Ile Gly Glu Arg Asn Gly Ile Thr Pro Leu Gly Gly Leu Met Ala
Arg Met Ile Val Ala Ala Pro Glu Tyr Thr Lys Ser Lys Tyr Lys Leu
His Lys Ile Arg Asp Ile Glu Asn Leu Ile Ala Glu Ala Val Glu Val
Asn Ile Pro Phe Asn Asn Pro Ile Thr Gly Phe Cys Ala Phe Thr His
Lys Ala Gly Ile His Ala Lys Ala Ile Leu Ala Asn Pro Ser Thr Tyr
Glu Ile Leu Asp Pro His Asp Phe Gly Met Lys Arg Tyr Ile His Phe
Ala Asn Arg Leu Thr Gly Trp Asn Ala Ile Lys Ser Arg Val Asp Gln
Leu Asn Leu Asn Leu Thr Asp Asp Gln Val Lys Glu Val Thr Ala Lys
Ile Lys Lys Leu Gly Asp Ile Arg Pro Leu Asn Ile Asp Asp Val Asp
Ser Ile Ile Lys Asp Phe His Ala Glu Val Ser Thr Pro Gln Leu Arg
Ala Val Arg Arg Asp Asp Asn Asp Val Asn Asp Ile Asp Ile Gln Glu
Pro Ser Asn Lys Lys Thr Lys Val Glu
SEQ ID NO: 49
PRT - Schizosaccharomyces pombe
Met Ser Val Ser Glu Ala Asn Gly Thr Glu Thr Ile Lys Pro Pro Met
Asn Gly Asn Pro Tyr Gly Pro Asn Pro Ser Asp Phe Leu Ser Arg Val
Asn Asn Phe Ser Ile Ile Glu Ser Thr Leu Arg Glu Gly Glu Gln Phe
Ala Asn Ala Phe Phe Asp Thr Glu Lys Lys Ile Gln Ile Ala Lys Ala
Leu Asp Asn Phe Gly Val Asp Tyr Ile Glu Leu Thr Ser Pro Val Ala
Ser Glu Gln Ser Arg Gln Asp Cys Glu Ala Ile Cys Lys Leu Gly Leu
Lys Cys Lys Ile Leu Thr His Ile Arg Cys His Met Asp Asp Ala Arg
Val Ala Val Glu Thr Gly Val Asp Gly Val Asp Val Val Ile Gly Thr
Ser Gln Tyr Leu Arg Lys Tyr Ser His Gly Lys Asp Met Thr Tyr Ile
Ile Asp Ser Ala Thr Glu Val Ile Asn Phe Val Lys Ser Lys Gly Ile
Glu Val Arg Phe Ser Ser Glu Asp Ser Phe Arg Ser Asp Leu Val Asp
Leu Leu Ser Leu Tyr Lys Ala Val Asp Lys Ile Gly Val Asn Arg Val
Gly Ile Ala Asp Thr Val Gly Cys Ala Thr Pro Arg Gln Val Tyr Asp
Leu Ile Arg Thr Leu Arg Gly Val Val Ser Cys Asp Ile Glu Cys His
Phe His Asn Asp Thr Gly Met Ala Ile Ala Asn Ala Tyr Cys Ala Leu
Glu Ala Gly Ala Thr His Ile Asp Thr Ser Ile Leu Gly Ile Gly Glu
Arg Asn Gly Ile Thr Pro Leu Gly Ala Leu Leu Ala Arg Met Tyr Val
Thr Asp Arg Glu Tyr Ile Thr His Lys Tyr Lys Leu Asn Gln Leu Arg
Glu Leu Glu Asn Leu Val Ala Asp Ala Val Glu Val Gln Ile Pro Phe
Asn Asn Tyr Ile Thr Gly Met Cys Ala Phe Thr His Lys Ala Gly Ile
His Ala Lys Ala Ile Leu Ala Asn Pro Ser Thr Tyr Glu Ile Leu Lys
Pro Glu Asp Phe Gly Met Ser Arg Tyr Val His Val Gly Ser Arg Leu
Thr Gly Trp Asn Ala Ile Lys Ser Arg Ala Glu Gln Leu Asn Leu His
Leu Thr Asp Ala Gln Ala Lys Glu Leu Thr Val Arg Ile Lys Lys Leu
Ala Asp Val Arg Thr Leu Ala Met Asp Asp Val Asp Arg Val Leu Arg
Glu Tyr His Ala Asp Leu Ser Asp Ala Asp Arg Ile Thr Lys Glu Ala
Ser Ala
SEQ ID NO: 50
PRT - Aspergillus niger
Met Cys Pro Gly Ala Asp His Glu Pro Asn Gly Gln Ala Asn Val Ala
Asn Gly Asn Gly Asn Asn Gly Glu His Pro Gly Phe Thr Ala Val Glu
Thr Arg Gln Asn Pro His Pro Ser Val Ser Arg Asn Pro Tyr Gly His
Asn Val Gly Val Thr Asp Phe Leu Ser Asn Val Ser Arg Phe Gln Ile
Ile Glu Ser Thr Leu Arg Glu Gly Glu Gln Phe Ala Asn Ala Phe Phe
Asp Thr Glu Lys Lys Ile Glu Ile Ala Lys Ala Leu Asp Glu Phe Gly
Val Asp Tyr Ile Glu Leu Thr Ser Pro Cys Ala Ser Glu Gln Ser Arg
Lys Asp Cys Glu Ala Ile Cys Lys Leu Gly Leu Lys Ala Lys Ile Leu
Thr His Ile Arg Cys His Met Asp Asp Ala Arg Ile Ala Val Glu Thr
Gly Val Asp Gly Val Asp Val Val Ile Gly Thr Ser Ser Tyr Leu Arg
Glu His Ser His Gly Lys Asp Met Thr Tyr Ile Lys Asn Thr Ala Ile
Glu Val Ile Glu Phe Val Lys Ser Lys Gly Ile Glu Ile Arg Phe Ser
Ser Glu Asp Ser Phe Arg Ser Asp Leu Val Asp Leu Leu Ser Ile Tyr
Ser Ala Val Asp Lys Val Gly Val Asn Arg Val Gly Ile Ala Asp Thr
Val Gly Cys Ala Ser Pro Arg Gln Val Tyr Glu Leu Val Arg Val Leu
Arg Gly Val Val Ser Cys Asp Ile Glu Thr His Phe His Asn Asp Thr
Gly Cys Ala Ile Ala Asn Ala Tyr Cys Ala Leu Glu Ala Gly Ala Thr
His Ile Asp Thr Ser Val Leu Gly Ile Gly Glu Arg Asn Gly Ile Thr
Pro Leu Gly Gly Leu Met Ala Arg Met Met Val Ala Asp Pro Glu Tyr
Val Lys Ser Lys Tyr Arg Leu Glu Lys Leu Lys Asp Ile Glu Asp Leu
Val Ala Glu Ala Val Glu Val Asn Ile Pro Phe Asn Asn Tyr Ile Thr
Gly Phe Cys Ala Phe Thr His Lys Ala Gly Ile His Ala Lys Ala Ile
Leu Asn Asn Pro Ser Thr Tyr Glu Ile Ile Asn Pro Ala Asp Phe Gly
Met Ser Arg Tyr Val His Phe Ala Ser Arg Leu Thr Gly Trp Asn Ala
Ile Lys Ser Arg Ala Gln Gln Leu Lys Ile Glu Met Thr Asp Asp Gln
Tyr Lys Glu Cys Thr Ala Lys Ile Lys Ala Leu Ala Asp Ile Arg Pro
Ile Ala Val Asp Asp Ala Asp Ser Ile Ile Arg Ala Tyr Tyr Arg Asn
Leu Lys Leu Gly Glu Asn Lys Pro Leu Leu Asp Leu Thr Ala Asp Glu
Gln Ala Gln Phe Ala Ala Lys Glu Lys Glu Leu Ala Ala Gln Ala Ser
Ala
SEQ ID NO: 51
PRT - Emericella nidulans
Met Cys Pro Gly Asp His Pro Gly Phe Thr Ala Val Gln Thr Arg Gln
Asn Pro His Pro Ser Arg Asn Pro Tyr Gly His Asn Val Gly Val Thr
Asp Phe Leu Ser Asn Val Ser Arg Phe Lys Ile Ile Glu Ser Thr Leu
Arg Glu Gly Glu Gln Phe Ala Asn Ala Phe Phe Asp Thr Gln Lys Lys
Ile Glu Ile Ala Lys Ala Leu Asp Glu Phe Gly Val Asp Tyr Ile Glu
Leu Thr Ser Pro Cys Ala Ser Glu Gln Ser Arg Leu Asp Cys Glu Ala
Ile Cys Lys Leu Gly Leu Lys Ala Lys Ile Leu Thr His Ile Arg Cys
His Met Asp Asp Ala Arg Val Ala Val Glu Thr Gly Val Asp Gly Val
Asp Val Val Ile Gly Thr Ser Ser Tyr Leu Arg Glu His Ser His Gly
Lys Asp Met Thr Tyr Ile Lys Asn Thr Ala Ile Glu Val Ile Glu Phe
Val Lys Ser Lys Gly Ile Glu Ile Arg Phe Ser Ser Glu Asp Ser Phe
Arg Ser Asp Leu Val Asp Leu Leu Ser Ile Tyr Ser Ala Val Asp Gln
Val Gly Val Asn Arg Val Gly Ile Ala Asp Thr Val Gly Cys Ala Ser
Pro Arg Gln Val Tyr Glu Leu Ile Arg Val Leu Arg Gly Val Val Ser
Cys Asp Ile Glu Thr His Phe His Asn Asp Thr Gly Cys Ala Ile Ala
Asn Ala Tyr Cys Ala Leu Glu Ala Gly Ala Thr His Ile Asp Thr Ser
Val Leu Gly Ile Gly Glu Arg Asn Gly Ile Thr Pro Leu Gly Gly Leu
Met Ala Arg Met Met Val Ala Asp Pro Gln Tyr Val Lys Ser Lys Tyr
Lys Leu Glu Lys Leu Lys Asp Ile Glu Asp Leu Val Ala Glu Ala Val
Glu Val Asn Ile Pro Phe Asn Asn Tyr Ile Thr Gly Phe Cys Ala Phe
Thr His Lys Ala Gly Ile His Ala Lys Ala Ile Leu Asn Asn Pro Ser
Thr Tyr Glu Ile Ile Asn Pro Ala Asp Phe Gly Met Ser Arg Tyr Val
His Phe Ala Ser Arg Leu Thr Gly Trp Asn Ala Ile Lys Ser Arg Ala
Gln Gln Leu Asn Val His Met Thr Asp Asp Gln Tyr Lys Glu Cys Thr
Ala Lys Ile Lys Ala Leu Ala Asp Ile Arg Pro Ile Ala Ile Asp Asp
Ala Asp Ser Ile Ile Arg Ala Tyr Tyr Arg Asn Leu Ser Ser Gly Glu
Asn Lys Pro Leu Met Asp Leu Thr Ala Asp Glu His Ala Gln Phe Leu
Ala Lys Glu Lys Glu Leu Thr Glu Ser Gly Thr Ala Leu
SEQ ID NO: 52
PRT - Penicillium chrysogenum
Met Val Leu Leu Pro Pro Ser Leu Pro Val Cys Gln Leu Lys Val Thr
Ala Pro Glu Phe Pro Ser Asn Phe Tyr Leu Asp Gly Asp His Ser Gly
Phe Val Gly Ile Glu Thr Arg Gln Asn Pro His Pro Ser Ala Ser Arg
Asn Pro Tyr Gly His Asp Ala Gly Val Thr Asp Phe Leu Ser Asn Val
Ser Arg Phe Gln Ile Ile Glu Ser Thr Leu Arg Glu Gly Glu Gln Phe
Ala Asn Ala Phe Phe Asp Thr Ala Lys Lys Ile Glu Ile Ala Lys Ala
Leu Asp Asp Phe Gly Val Asp Tyr Ile Glu Leu Thr Ser Pro Cys Ala
Ser Glu Gln Ser Arg Ala Asp Cys Glu Ala Ile Cys Lys Leu Gly Leu
Lys Ala Lys Ile Leu Thr His Ile Arg Cys His Met Asp Asp Ala Arg
Ile Ala Val Glu Thr Gly Val Asp Gly Val Asp Val Val Ile Gly Thr
Ser Ser Tyr Leu Arg Glu His Ser His Gly Lys Asp Met Thr Tyr Ile
Lys Asn Ala Ala Ile Glu Val Ile Glu Phe Val Lys Ser Lys Gly Ile
Glu Ile Arg Phe Ser Ser Glu Asp Ser Phe Arg Ser Asp Leu Val Asp
Leu Leu Ser Ile Tyr Ser Ala Val Asp Lys Val Gly Val Asn Arg Val
Gly Ile Ala Asp Thr Val Gly Cys Ala Ser Pro Arg Gln Val Tyr Glu
Leu Val Arg Val Leu Arg Gly Val Val Gly Cys Asp Ile Glu Thr His
Phe His Asn Asp Thr Gly Cys Ala Ile Ala Asn Ala Phe Cys Ala Leu
Glu Ala Gly Ala Thr His Ile Asp Thr Ser Val Leu Gly Ile Gly Glu
Arg Asn Gly Ile Thr Pro Leu Gly Gly Leu Met Ala Arg Met Met Val
Ala Asp Arg Glu Tyr Val Lys Ser Lys Tyr Lys Leu Glu Lys Leu Lys
Glu Ile Glu Asp Leu Val Ala Glu Ala Val Glu Val Asn Ile Pro Phe
Asn Asn Tyr Ile Thr Gly Phe Cys Ala Phe Thr His Lys Ala Gly Ile
His Ala Lys Ala Ile Leu Asn Asn Pro Ser Thr Tyr Glu Ile Ile Asn
Pro Ala Asp Phe Gly Met Ser Arg Tyr Val His Phe Ala Ser Arg Leu
Thr Gly Trp Asn Ala Ile Lys Ser Arg Ala Gln Gln Leu Lys Leu Glu
Met Thr Asp Thr Gln Tyr Lys Glu Cys Thr Ala Lys Ile Lys Ala Met
Ala Asp Ile Arg Pro Ile Ala Val Asp Asp Ala Asp Ser Ile Ile Arg
Ala Tyr His Arg Asn Leu Lys Ser Gly Glu Asn Lys Pro Leu Leu Asp
Leu Thr Ala Glu Glu Gln Ala Ala Phe Ala Ala Lys Glu Lys Glu Leu
Leu Glu Ala Gln Ala Ala Gly Leu Pro Val
SEQ ID NO: 53
PRT - Yarrowia lipolytica
Met Cys Ala Thr Asp Asn Ala Pro Ala Ala Asn Ala Ala Pro Glu Lys
Pro Ser Asn Val Gly Val Glu Val Gly His Thr Gly Glu Gln Thr Asn
Pro Tyr Gly Ala Asn Pro Ala Asp Phe Leu Ser Asn Val Ser Lys Phe
Gln Leu Ile Glu Ser Thr Leu Arg Glu Gly Glu Gln Phe Ala Ser Ala
Phe Phe Asp Thr Glu Thr Lys Ile Glu Ile Ala Lys Ala Leu Asp Asp
Phe Gly Val Asp Tyr Ile Glu Leu Thr Ser Pro Ala Ala Ser Glu Gln
Ser Arg Ser Asp Cys Glu Ala Ile Cys Lys Leu Gly Leu Lys Ala Lys
Ile Leu Thr His Ile Arg Cys His Met Asp Asp Ala Arg Leu Ala Val
Ser Thr Gly Val Asp Gly Val Asp Val Val Ile Gly Thr Ser Gln Phe
Leu Arg Gln Tyr Ser His Gly Lys Asp Met Asn Tyr Ile Ala Gln Ser
Ala Val Glu Val Ile Glu Phe Val Lys Ser His Gly Ile Glu Ile Arg
Phe Ser Ser Glu Asp Ser Phe Arg Ser Asp Leu Val Asp Leu Leu Asn
Ile Tyr Arg Thr Val Asp Lys Ile Gly Val Asn Arg Val Gly Ile Ala
Asp Thr Val Gly Cys Ala Asn Pro Arg Gln Val Tyr Glu Leu Val Arg
Thr Leu Lys Ser Val Val Ser Cys Asp Ile Glu Cys His Phe His Asn
Asp Thr Gly Cys Ala Ile Ala Asn Ala Tyr Thr Ala Leu Glu Ala Gly
Ala Asn Leu Ile Asp Val Ser Val Leu Gly Ile Gly Glu Arg Asn Gly
Ile Thr Ser Leu Gly Gly Leu Met Ala Arg Met Ile Ala Ala Asp Arg
Asp Tyr Val Leu Ser Lys Tyr Lys Leu His Lys Leu Arg Asp Leu Glu
Asn Leu Val Ala Asp Ala Val Gln Val Asn Ile Pro Phe Asn Asn Pro
Ile Thr Gly Phe Cys Ala Phe Thr His Lys Ala Gly Ile His Ala Lys
Ala Ile Leu Ala Asn Pro Ser Thr Tyr Glu Ile Leu Asn Pro Ala Asp
Phe Gly Leu Thr Arg Tyr Ile His Phe Ala Asn Arg Leu Thr Gly Trp
Asn Ala Ile Lys Ser Arg Val Asp Gln Leu Asn Leu His Leu Thr Asp
Ala Gln Cys Lys Asp Val Thr Ala Lys Ile Lys Lys Leu Gly Asp Val
Arg Ser Leu Asn Ile Asp Asp Val Asp Ser Ile Ile Arg Glu Phe His
Ala Asp Val Thr Ser Thr Pro Thr Val Ala Ala Thr Glu Gly Pro Ala
Val Glu Asp Glu Pro Ala Ala Lys Lys Ala Lys Thr Glu Glu
SEQ ID NO: 54
PRT - Phanerochaete chrysosporium
Ile Pro Gln Thr Val Ile Glu Lys Val Val Gln Lys Tyr Ala Val Gly
Leu Pro Gly Asp Lys Val Val Lys Ala Gly Asp Tyr Val Met Ile Arg
Pro Glu His Val Met Thr His Asp Asn Thr Gly Pro Val Ile Ser Lys
Phe Lys Ser Ile Gly Ala Thr Arg Ile Tyr Asn Pro Lys Gln Val Val
Phe Thr Leu Asp His Asp Val Gln Asn Lys Ser Glu Lys Asn Leu Lys
Lys Tyr Ala Thr Ile Glu Ala Phe Ala Arg Thr His Gly Ile Asp Phe
Tyr Pro Ala Gly Arg Gly Ile Gly His Gln Val Leu Val Glu Glu Gly
Tyr Ala Phe Pro His Thr Leu Thr Val Ala Ser Asp Ser His Ser Asn
Met Tyr Gly Gly Val Gly Cys Val Gly Thr Pro Ile Val Arg Thr Asp
Ala Ala Ala Leu Trp Ala Thr Gly Gln Thr Trp Trp Gln Val Pro Arg
Met Val Lys Val Glu Phe Lys Gly Arg Leu Ala Pro Gly Val Ser Gly
Lys Asp Val Ile Val Ala Leu Cys Gly Ser Phe Asn Lys Asp Glu Val
Leu Asn Ala Ala Ile Glu Phe Ser Gly Glu Gly Val Gln His Leu Thr
Val Asp Glu Arg Leu Thr Ile Ala Asn Met Thr Thr Glu Trp Gly Ala
Leu Val Gly Val Phe Pro Val Asp Asp Val Thr Leu Ser Trp Tyr Glu
Arg Met Leu Lys Lys Leu Glu Leu Arg Thr Phe Ser Thr Pro Ala Leu
Gly Ser Ser Ile Pro Pro Pro Pro Glu His Pro Arg Ile Asn Arg Ala
Arg Leu Asp Ala Leu Arg Ala Asn Asn Leu Arg Ser Asp Ala Asp Ala
Glu Tyr Ser Ser His Leu Val Phe Asp Leu Ser Thr Leu Val Pro Tyr
Val Ser Gly Pro Asn Ser Val Lys Val Ala Asn Pro Leu Pro Lys Leu
Glu Glu Ala Lys Ile Lys Ile Asn Lys Ala Tyr Leu Leu Ser Cys Thr
Asn Ala Arg Ala Ser Asp Ile Ala Ala Ala Ala Ala Val Ile Lys Gly
His Lys Val His Pro Asp Val Gln Phe Tyr Phe Ala Pro Ala Ser Ser
Glu Val Gln Arg Glu Ala Glu Gln Ser Gly Asp Trp Glu Thr Leu Ile
Gly Ala Gly Ala Lys Pro Leu Pro Ala Gly Cys Gly Pro Cys Ile Gly
Leu Gly Thr Gly Leu Leu Glu Glu Gly Glu Val Gly Ile Ser Ala Thr
Asn Arg Asn Tyr Lys Gly Arg Met Gly His Pro Leu Ala Gln Ala Tyr
Leu Ala Ser Pro Ala Val Val Ala Ala Ser Ala Ile Lys Gly Tyr Ile
Ala Gly Pro Asp Ser Leu Asp Pro Ser Lys Leu Pro Pro Ala Gly Ala
Pro Thr Phe Ser Ile Val Asn Ser Pro Ser Ser Gly Ala Lys Ala Ser
Gln Lys Glu Pro Val Leu Val Gly Phe Pro Glu Thr Phe Ala Gly Pro
Leu Leu Phe Ala Pro Gln Asp Asn Leu Asn Thr Asp Gly Ile Tyr Pro
Gly Lys Tyr Thr Tyr Gln Asp Asp Ile Thr Leu Glu Arg Gln Ala Glu
Val Val Met Glu Asn Tyr Asp Pro Thr Phe Ala Gln Leu Asp Ala His
Thr Lys Arg Gly Val Val Leu Val Ser Gly Tyr Asn Phe Gly Thr Gly
Ser Ser Arg Glu Gln Ala Ala Thr Ala Leu Lys Ser Ala Gly Ile Pro
Ile Val Ile Ala Gly Ser Phe Gly Asp Ile Phe Lys Arg Asn Ala Ile
Asn Asn Gly Leu Val Cys Val Glu Ser Pro Glu Leu Val Ala Asp Leu
Thr Ala Gln Phe Ala Lys Asp Gly Lys Arg Gly Ala Gly Gly Lys Glu
Gly Glu Leu Thr Val Asn Lys Gly Leu Ser Ala Glu Val Lys Val Val
Asp Gly Ala Leu His Val Thr Phe Pro Asp Gly Lys Thr Lys Thr Tyr
Thr Ile Gln Pro Val Gly Ala Ser Val Gln Glu Leu Trp Leu Cys Gly
Gly Leu Glu Gly Tyr Val Leu Lys Ala Ile Gln Ala Glu Asn Phe
SEQ ID NO: 55
PRT - Schizosaccharomyces pombe
Met Asp Ser Gly Glu Met His His Pro Tyr Gln Ala Phe Ser Lys Val
Gly Lys Cys Glu Ile Ser Gln Thr Asn Pro Ser Phe Ser Ser Gly Met
Arg Cys Leu Val Arg Ser Ala Asp Ile Gln Phe Lys Gly Ile Cys Gly
Leu Thr Arg Gly Phe Ala Ser Phe Asn Lys Pro Pro Gln Thr Ile Thr
Glu Lys Ile Val Gln Lys Phe Ala Gln Asn Ile Pro Glu Asn Lys Tyr
Val Arg Ser Gly Asp Tyr Val Thr Ile Lys Pro Lys His Cys Met Ser
His Asp Asn Ser Trp Pro Val Ala Leu Lys Phe Met Gly Ile Gly Ala
Lys Lys Val Phe Asp Asn Arg Gln Ile Val Cys Thr Leu Asp His Asp
Val Gln Asn Lys Ser Glu Ala Asn Leu Arg Lys Tyr Lys Asn Ile Glu
Ser Phe Ala Lys Gly Gln Gly Ile Asp Phe Tyr Pro Ala Gly Arg Gly
Ile Gly His Gln Ile Met Val Glu Gln Gly Tyr Ala Met Pro Gly Ser
Met Ala Val Ala Ser Asp Ser His Ser Asn Thr Tyr Gly Gly Val Gly
Cys Leu Gly Thr Pro Ile Val Arg Thr Asp Ala Ala Ala Ile Trp Ala
Thr Gly Gln Thr Trp Trp Gln Ile Pro Pro Ile Ala Arg Val Asn Leu
Val Gly Gln Leu Pro Lys Gly Leu Ser Gly Lys Asp Ile Ile Val Ser
Leu Cys Gly Ala Phe Asn His Asp Glu Val Leu Asn His Ala Ile Glu
Phe Tyr Gly Glu Gly Leu Asn Ser Leu Ser Ile Glu Ser Arg Leu Thr
Ile Ala Asn Met Thr Thr Glu Trp Gly Ala Leu Ser Gly Leu Phe Pro
Thr Asp Glu Lys Leu Leu Ala Trp Tyr Glu Asp Arg Leu Lys Phe Leu
Gly Pro Asn His Pro Arg Val Asn Arg Glu Thr Leu Asp Ala Ile Lys
Ala Ser Pro Ile Leu Ala Asp Glu Gly Ala Phe Tyr Ala Lys His Leu
Ile Leu Asp Leu Ser Thr Leu Ser Pro Ala Val Ser Gly Pro Asn Ser
Val Lys Val Tyr Asn Ser Ala Ala Thr Leu Glu Lys Lys Asp Ile Leu
Ile Lys Lys Ala Tyr Leu Val Ser Cys Thr Asn Gly Arg Leu Ser Asp
Ile His Asp Ala Ala Glu Thr Val Lys Gly Lys Lys Val Ala Asp Gly
Val Glu Phe Tyr Val Gly Ala Ala Ser Ser Glu Val Glu Ala Ala Ala
Gln Lys Asn Gly Asp Trp Gln Thr Leu Ile Asp Ser Gly Ala Arg Thr
Leu Pro Ala Gly Cys Gly Pro Cys Ile Gly Leu Gly Thr Gly Leu Leu
Lys Asp Gly Glu Val Gly Ile Ser Ala Thr Asn Arg Asn Phe Lys Gly
Arg Met Gly Ser Arg Glu Ala Leu Ala Tyr Leu Ala Ser Pro Ala Val
Val Ala Ala Ser Ala Ile Ala Gly Lys Ile Val Ala Pro Glu Gly Phe
Lys Asn Ala Val Ser Leu Val Ser Ala Val Asp Ile Thr Asp Lys Val
Asn Lys Gln Thr Ala Ser Lys Ser Ser Thr Glu Ala Val Asp Ser Glu
Thr Ala Ile Ile Asp Gly Phe Pro Ser Ile Val Ala Gly Glu Ile Val
Phe Cys Asp Ala Asp Asn Leu Asn Thr Asp Gly Ile Tyr Pro Gly Arg
Tyr Thr Tyr Arg Asp Asp Ile Thr Lys Glu Glu Met Ala Lys Val Cys
Met Glu Asn Tyr Asp Ser Glu Phe Gly Lys Lys Thr Lys Lys Asp Asp
Ile Leu Val Ser Gly Phe Asn Phe Gly Thr Gly Ser Ser Arg Glu Gln
Ala Ala Thr Ala Ile Leu Ser Arg Gly Ile Pro Leu Val Val Gly Gly
Ser Phe Ser Asp Ile Phe Lys Arg Asn Ser Ile Asn Asn Ala Leu Leu
Ala Ile Gln Leu Pro Asp Leu Val Gln Lys Leu Arg Thr Ala Phe Ala
Asn Glu Ser Lys Glu Leu Thr Arg Arg Thr Gly Trp His Leu Lys Trp
Asp Val Arg Lys Ser Thr Val Thr Val Thr Thr Ser Asp Asn Lys Glu
Met Ser Trp Lys Ile Gly Glu Leu Gly Asn Ser Val Gln Ser Leu Phe
Val Arg Gly Gly Leu Glu Gly Trp Val Lys His Glu Ile Ser Lys Ser
Asn
SEQ ID NO: 56
PRT - Kluyveromyces lactis
Met Phe Arg Val Gln Arg Leu Arg Met Phe Ser Thr Ser Arg Ala Leu
Tyr Ala Gly Gln Asn Met Thr Glu Lys Ile Val Gln Arg His Ala Val
Gly Leu Pro Glu Gly Lys Thr Val Val Ser Gly Asp Tyr Val Ser Ile
Lys Pro Ala His Cys Met Ser His Asp Asn Ser Trp Pro Val Ala Leu
Lys Phe Met Gly Leu Gly Ala Ser Thr Ile Lys Asn Pro Arg Gln Val
Val Asn Thr Leu Asp His Asp Val Gln Asn Lys Ser Glu Lys Asn Leu
Thr Lys Tyr Lys Asn Ile Glu Asn Phe Ala Lys Lys His Gly Ile Asp
Phe Tyr Pro Ala Gly Arg Gly Ile Gly His Gln Ile Met Ile Glu Glu
Gly Tyr Ala Phe Pro Leu Thr Met Thr Val Ala Ser Asp Ser His Ser
Asn Thr Tyr Gly Gly Ile Gly Ala Leu Gly Thr Pro Ile Val Arg Thr
Asp Ala Ala Ala Ile Trp Ala Thr Gly Gln Thr Trp Trp Gln Ile Pro
Pro Val Ala Gln Val Glu Leu Lys Gly Glu Leu Pro Ala Gly Ile Ser
Gly Lys Asp Ile Ile Val Ala Leu Cys Gly Val Phe Asn Gln Asp Gln
Val Leu Asn His Ala Ile Glu Phe Thr Gly Asp Ser Leu Asp Lys Ile
Pro Ile Asp Tyr Arg Leu Thr Ile Ala Asn Met Thr Thr Glu Trp Gly
Ala Leu Ser Gly Leu Phe Pro Val Asp Asn Val Leu Leu Asp Phe Tyr
Arg Asn Arg Leu Thr Lys Val Gly Asn Asn His Pro Arg Ile Asn Glu
Ala Arg Ile Asn Glu Leu Gln Ala Lys Ser Asp Ser Leu Gln Ala Asp
Pro Asp Ala Lys Tyr Ala Lys Lys Leu Ile Ile Asp Leu Ser Thr Leu
Thr His Tyr Val Ser Gly Pro Asn Ser Val Lys Ile Ser Ser Thr Val
Asp Asp Leu Ser Lys Gln Asp Ile Lys Val Asn Lys Ala Tyr Leu Val
Ser Cys Thr Asn Ser Arg Leu Ser Asp Leu Glu Ser Ala Ala Asn Val
Val Cys Pro Ser Gly Asp Ile Asn Gln Val His Lys Val Ala Glu Gly
Val Glu Phe Tyr Ile Ala Ala Ala Ser Ser Glu Val Glu Ala Glu Ala
Arg Ala Thr Gly Ala Trp Gln Lys Leu Leu Asn Ala Gly Cys Leu Pro
Leu Pro Ala Gly Cys Gly Pro Cys Ile Gly Leu Gly Thr Gly Leu Leu
Glu Glu Gly Gln Val Gly Ile Ser Ala Thr Asn Arg Asn Phe Lys Gly
Arg Met Gly Ser Lys Asp Ala Leu Ala Tyr Leu Ala Ser Pro Ser Val
Val Ala Ala Ser Ala Ile Leu Gly Lys Ile Gly Ser Pro Ala Glu Val
Leu Gly Thr Lys Asp Pro Asn Phe Thr Gly Val Val Ala Thr Val Glu
Asp Ala Pro Ala Thr Ser Ala Asp Gly Lys Asp Val Ala Asp Glu Ser
Gly Ala Ser Gly Ser Val Glu Ile Leu Glu Gly Phe Pro Ser Glu Ile
Ser Gly Glu Leu Val Leu Cys Asp Ala Asp Asn Ile Asn Thr Asp Gly
Ile Tyr Pro Gly Lys Tyr Thr Tyr Gln Asp Asp Val Pro Lys Glu Thr
Met Ala Lys Val Cys Met Glu Asn Tyr Asp Pro Asp Phe Gln Thr Lys
Ala Asn Pro Gly Asp Ile Leu Ile Ser Gly Phe Asn Phe Gly Thr Gly
Ser Ser Arg Glu Gln Ala Ala Thr Ala Ile Leu Ala Lys Gly Ile Lys
Leu Val Val Ser Gly Ser Phe Gly Asn Ile Phe Phe Arg Asn Ser Ile
Asn Asn Ala Leu Leu Thr Leu Glu Ile Pro Ala Leu Ile Asn Met Leu
Arg Asp Arg Tyr Lys Asp Ala Pro Lys Glu Leu Thr Arg Arg Thr Gly
Trp Phe Leu Lys Trp Asp Val Ser Gln Ala Lys Val Tyr Val Thr Glu
Gly Ser Val Asn Gly Pro Ile Val Leu Glu Gln Lys Val Gly Glu Leu
Gly Lys Asn Leu Gln Glu Ile Ile Val Lys Gly Gly Leu Glu Ser Trp
Val Lys Ser Gln Leu
SEQ ID NO: 57
PRT - Saccharomyces cerevisiae
Met Leu Arg Ser Thr Thr Phe Thr Arg Ser Phe His Ser Ser Arg Ala
Trp Leu Lys Gly Gln Asn Leu Thr Glu Lys Ile Val Gln Ser Tyr Ala
Val Asn Leu Pro Glu Gly Lys Val Val His Ser Gly Asp Tyr Val Ser
Ile Lys Pro Ala His Cys Met Ser His Asp Asn Ser Trp Pro Val Ala
Leu Lys Phe Met Gly Leu Gly Ala Thr Lys Ile Lys Asn Pro Ser Gln
Ile Val Thr Thr Leu Asp His Asp Ile Gln Asn Lys Ser Glu Lys Asn
Leu Thr Lys Tyr Lys Asn Ile Glu Asn Phe Ala Lys Lys His His Ile
Asp His Tyr Pro Ala Gly Arg Gly Ile Gly His Gln Ile Met Ile Glu
Glu Gly Tyr Ala Phe Pro Leu Asn Met Thr Val Ala Ser Asp Ser His
Ser Asn Thr Tyr Gly Gly Leu Gly Ser Leu Gly Thr Pro Ile Val Arg
Thr Asp Ala Ala Ala Ile Trp Ala Thr Gly Gln Thr Trp Trp Gln Ile
Pro Pro Val Ala Gln Val Glu Leu Lys Gly Gln Leu Pro Gln Gly Val
Ser Gly Lys Asp Ile Ile Val Ala Leu Cys Gly Leu Phe Asn Asn Asp
Gln Val Leu Asn His Ala Ile Glu Phe Thr Gly Asp Ser Leu Asn Ala
Leu Pro Ile Asp His Arg Leu Thr Ile Ala Asn Met Thr Thr Glu Trp
Gly Ala Leu Ser Gly Leu Phe Pro Val Asp Lys Thr Leu Ile Asp Trp
Tyr Lys Asn Arg Leu Gln Lys Leu Gly Thr Asn Asn His Pro Arg Ile
Asn Pro Lys Thr Ile Arg Ala Leu Glu Glu Lys Ala Lys Ile Pro Lys
Ala Asp Lys Asp Ala His Tyr Ala Lys Lys Leu Ile Ile Asp Leu Ala
Thr Leu Thr His Tyr Val Ser Gly Pro Asn Ser Val Lys Val Ser Asn
Thr Val Gln Asp Leu Ser Gln Gln Asp Ile Lys Ile Asn Lys Ala Tyr
Leu Val Ser Cys Thr Asn Ser Arg Leu Ser Asp Leu Gln Ser Ala Ala
Asp Val Val Cys Pro Thr Gly Asp Leu Asn Lys Val Asn Lys Val Ala
Pro Gly Val Glu Phe Tyr Val Ala Ala Ala Ser Ser Glu Ile Glu Ala
Asp Ala Arg Lys Ser Gly Ala Trp Glu Lys Leu Leu Lys Ala Gly Cys
Ile Pro Leu Pro Ser Gly Cys Gly Pro Cys Ile Gly Leu Gly Ala Gly
Leu Leu Glu Pro Gly Glu Val Gly Ile Ser Ala Thr Asn Arg Asn Phe
Lys Gly Arg Met Gly Ser Lys Asp Ala Leu Ala Tyr Leu Ala Ser Pro
Ala Val Val Ala Ala Ser Ala Val Leu Gly Lys Ile Ser Ser Pro Ala
Glu Val Leu Ser Thr Ser Glu Ile Pro Phe Ser Gly Val Lys Thr Glu
Ile Ile Glu Asn Pro Val Val Glu Glu Glu Val Asn Ala Gln Thr Glu
Ala Pro Lys Gln Ser Val Glu Ile Leu Glu Gly Phe Pro Arg Glu Phe
Ser Gly Glu Leu Val Leu Cys Asp Ala Asp Asn Ile Asn Thr Asp Gly
Ile Tyr Pro Gly Lys Tyr Thr Tyr Gln Asp Asp Val Pro Lys Glu Lys
Met Ala Gln Val Cys Met Glu Asn Tyr Asp Ala Glu Phe Arg Thr Lys
Val His Pro Gly Asp Ile Val Val Ser Gly Phe Asn Phe Gly Thr Gly
Ser Ser Arg Glu Gln Ala Ala Thr Ala Leu Leu Ala Lys Gly Ile Asn
Leu Val Val Ser Gly Ser Phe Gly Asn Ile Phe Ser Arg Asn Ser Ile
Asn Asn Ala Leu Leu Thr Leu Glu Ile Pro Ala Leu Ile Lys Lys Leu
Arg Glu Lys Tyr Gln Gly Ala Pro Lys Glu Leu Thr Arg Arg Thr Gly
Trp Phe Leu Lys Trp Asp Val Ala Asp Ala Lys Val Val Val Thr Glu
Gly Ser Leu Asp Gly Pro Val Ile Leu Glu Gln Lys Val Gly Glu Leu
Gly Lys Asn Leu Gln Glu Ile Ile Val Lys Gly Gly Leu Glu Gly Trp
Val Lys Ser Gln Leu
SEQ ID NO: 58
PRT - Aspergillus niger
Met Gln Ser Arg Leu Leu Pro Ser Gly Pro Gly Arg Arg Trp Ile Ser
Leu Arg Val Pro Asn Thr Pro Gln Arg Arg Ala Phe Ala Ser Thr Arg
Phe Leu Phe Gln Asp Val Phe Gln Ser Gln Leu Asp Asp Pro Ser Ser
Ala Ala Leu Phe Ser Ser Leu Gln Ser Ser Arg Ala Val Pro Gln Thr
Leu Thr Glu Lys Ile Val Gln Lys Tyr Ala Val Gly Leu Pro Asp Gly
Lys Phe Val Lys Ser Gly Asp Tyr Val Thr Ile Ala Pro His Arg Ile
Met Thr His Asp Asn Ser Trp Pro Val Ala Leu Lys Phe Met Ser Ile
Gly Ala Ser Lys Met His Asp Pro Asn Gln Val Val Met Thr Leu Asp
His Asp Val Gln Asn Lys Thr Glu Lys Asn Leu Gln Lys Tyr Arg Gln
Ile Glu Glu Phe Ala Lys Gln His Gly Val Glu Phe Tyr Pro Ala Gly
Arg Gly Ile Gly His Gln Ile Met Val Glu Glu Gly Phe Ala Trp Pro
Gly Thr Leu Val Val Ala Ser Asp Ser His Ser Asn Thr Tyr Gly Ala
Val Ala Ser Val Gly Thr Pro Ile Val Arg Thr Asp Ala Ala Ser Ile
Trp Ala Thr Gly Lys Thr Trp Trp Gln Ile Pro Pro Val Ala Lys Val
Thr Phe Thr Gly Ile Leu Pro Pro Gly Val Thr Gly Lys Asp Val Ile
Val Ala Leu Cys Gly Leu Phe Asp Lys Asp Asp Val Leu Asn His Ala
Ile Glu Phe Thr Gly Ser Glu Glu Thr Met Arg Ser Leu Pro Met Asp
Ser Arg Leu Thr Ile Ala Asn Met Thr Thr Glu Trp Gly Ala Leu Ser
Gly Leu Phe Pro Met Asp Gly Val Leu Lys Gly Trp Leu Lys Gly Lys
Ala Thr Thr Ala Ala Met Gly Leu Ala Asp Gly Pro Phe Lys Thr Leu
Ala Ala Arg Asn Phe Thr His Pro Ala Ile Glu Gln Leu Phe Val Asn
Pro Leu Thr Ala Asp Lys Gly Ala Lys Tyr Ala Lys Glu Leu Phe Leu
Asp Leu Ser Thr Leu Ser Pro Tyr Val Ser Gly Pro Asn Ser Val Lys
Ile Ala Thr Pro Leu Lys Glu Leu Glu Ala Gln Asp Ile Lys Val Asp
Lys Ala Tyr Leu Val Ser Cys Thr Asn Ser Arg Ala Ser Asp Ile Ala
Ala Ala Ala Lys Val Phe Lys Asp Ala Ala Glu Lys Asn Gly Gly Lys
Val Pro Lys Ile Ala Asp Gly Val Lys Phe Tyr Ile Ala Ala Ala Ser
Ile Pro Glu Gln Leu Ala Ala Glu Gly Ala Gly Asp Trp Gln Thr Leu
Leu Glu Ala Gly Ala Thr Ala Leu Pro Ala Gly Cys Gly Pro Cys Ile
Gly Leu Gly Thr Gly Leu Leu Glu Pro Gly Glu Val Gly Ile Ser Ala
Ser Asn Arg Asn Phe Lys Gly Arg Met Gly Ser Thr Glu Ala Lys Ala
Tyr Leu Gly Ser Pro Glu Ile Val Ala Ala Ser Ala Leu Ser Gly Lys
Leu Ser Gly Pro Gly Trp Tyr Gln Pro Pro Glu Gly Trp Thr Glu Val
Val Arg Gly Glu Gly Asp Gly Ile Arg Glu Glu Asp Arg Met Leu Asn
Thr Glu Gln Ala Leu Glu Lys Leu Leu Gly Gln Leu Asp Asp Leu Val
Ala Asp Gly Glu Lys Arg Phe Ala Pro Glu Glu Lys Val Glu Glu Glu
Gly Gly Leu Thr Glu Val Tyr Pro Gly Phe Pro Glu Arg Val Ser Gly
Glu Ile Val Phe Cys Asp Ala Asp Asn Leu Asn Thr Asp Ala Ile Tyr
Pro Gly Tyr Trp Thr Tyr Gln Asp Asn Val Pro Val Glu Lys Met Ala
Glu Val Cys Met Ser Asn Tyr Asp Lys Glu Phe Ala Ser Ile Ala Lys
Glu Gly Asp Ile Leu Val Val Gly Tyr Asn Phe Gly Cys Gly Ser Ser
Arg Glu Gln Ala Ala Thr Ala Leu Leu Ala Lys Gln Ile Pro Leu Val
Val Ser Gly Ser Phe Gly Asn Ile Phe Ser Arg Asn Ser Ile Asn Asn
Ala Leu Met Gly Leu Glu Val Pro Arg Leu Val Ser Arg Leu Arg Glu
Glu Phe Gly Asp Lys Gln Leu Thr Arg Arg Thr Gly Trp Thr Leu Thr
Trp Asp Val Arg Arg Ser Gln Ile Glu Ile Gln Glu Gly Gln Asn Gly
Pro Lys Trp Thr His Lys Val Gly Glu Leu Pro Pro Asn Val Gln Glu
Ile Ile Ala Lys Gly Gly Leu Glu Lys Trp Val Lys Asn Ala Ile Glu
Ala
SEQ ID NO: 59
PRT - Emericella nidulans
Met Gln Ser Arg Leu Val Ser Gln Ser Gly Leu Gly Arg Arg Trp Ala
Val Leu Arg Cys Ala Leu Ser Lys Thr Tyr Gln Arg Arg Thr Leu Thr
Ser Thr Arg Arg Gln Phe Gln Asp Val Phe Gln Ser Gln Leu Glu Asp
Pro Thr Ser Ala Ala Leu Phe Ser Ala Leu Asn Ser Ser Lys Ala Val
Pro Gln Thr Leu Thr Glu Lys Ile Val Gln Lys Tyr Ser Val Gly Leu
Pro Gln Gly Lys Phe Val Lys Ser Gly Asp Tyr Val Thr Ile Gln Pro
His Arg Cys Met Thr His Asp Asn Ser Trp Pro Cys Ala Leu Lys Phe
Met Ser Ile Gly Ala Ser Arg Leu His Asn Pro Asp Gln Ile Val Met
Thr Leu Asp His Asp Val Gln Asn Lys Ser Asp Lys Asn Leu Lys Lys
Tyr Arg Gln Ile Glu Glu Phe Ala Thr Gln His Gly Val Glu Phe Tyr
Pro Ala Gly Arg Gly Ile Gly His Gln Ile Met Ile Glu Glu Gly Phe
Ala Trp Pro Gly Thr Leu Ala Val Ala Ser Asp Ser His Ser Asn Met
Tyr Gly Gly Val Gly Cys Leu Gly Thr Pro Ile Val Arg Thr Asp Ala
Ala Ser Val Trp Ala Thr Gly Lys Thr Trp Trp Gln Ile Pro Pro Val
Ala Lys Val Thr Phe Lys Gly Val Leu Pro Pro Gly Val Thr Gly Lys
Asp Val Ile Val Ala Leu Cys Gly Leu Phe Asn Lys Asp Asp Val Leu
Asn His Ala Ile Glu Phe Thr Gly Ser Glu Glu Thr Met Arg Ser Leu
Ser Val Asp Thr Arg Leu Thr Ile Ala Asn Met Thr Thr Glu Trp Gly
Ala Leu Ser Gly Leu Phe Pro Ile Asp Ser Val Leu Lys Gly Trp Leu
Arg Gly Lys Ala Thr Thr Ala Ala Met Gly Leu Ala Asp Gly Pro Phe
Lys Thr Arg Ala Ala Glu Arg Phe Thr His Pro Leu Leu Glu Gln Leu
Phe Glu Asn Pro Leu Thr Ala Asp Lys Gly Ala Lys Tyr Ala Lys Glu
Leu Phe Leu Asp Leu Ser Ser Leu Ser Pro Tyr Val Ser Gly Pro Asn
Ser Val Lys Val Ala Thr Pro Leu Lys Glu Leu Glu Ala Gln Asn Ile
Lys Val Asp Lys Ala Tyr Leu Val Ser Cys Thr Asn Ser Arg Ala Ser
Asp Ile Ala Ala Ala Ala Lys Val Phe Lys Glu Ala Ala Glu Lys Asn
Gly Gly Lys Ile Pro Lys Ile Ala Asp Gly Val Lys Phe Tyr Ile Ala
Ala Ala Ser Ile Pro Glu Gln Leu Ala Ala Glu Gly Asn Gly Asp Trp
Gln Thr Leu Leu Glu Ala Gly Ala Thr Gln Leu Pro Ala Gly Cys Gly
Pro Cys Ile Gly Met Gly Gln Gly Leu Leu Glu Pro Gly Glu Val Gly
Ile Ser Ala Ser Asn Arg Asn Phe Lys Gly Arg Met Gly Ser Thr Glu
Ala Lys Ala Tyr Leu Gly Ser Pro Glu Val Val Ala Ala Ser Ala Leu
Ser Gly Lys Leu Ser Gly Pro Gly Trp Tyr Gln Thr Pro Glu Gly Trp
Thr Glu Val Ile Arg Gly Glu Gly Asp Gly Ile Arg Glu Glu Asp Arg
Met Leu Thr Asn Glu Glu Ala Leu Glu Lys Ile Ile Gly Gln Leu Asp
Asp Leu Val Ala Asp Gly Glu Lys Arg Phe Ala Ser Glu Thr Pro Ala
Val Glu Glu Ser Glu Gln Gly Leu Thr Glu Ile Tyr Pro Gly Phe Pro
Glu Arg Val Ser Gly Glu Leu Val Phe Cys Asp Ala Asp Asn Val Asn
Thr Asp Gly Ile Tyr Pro Gly Lys Tyr Thr Tyr Gln Asp Asp Val Pro
Pro Glu Thr Met Ala Arg Val Cys Met Glu Asn Tyr Asp Pro Glu Phe
Ser Thr Thr Ala Lys Glu Gly Asp Ile Leu Val Ser Gly Phe Asn Phe
Gly Cys Gly Ser Ser Arg Glu Gln Ala Ala Thr Ala Ile Leu Ala Lys
Lys Ile Pro Leu Val Val Ser Gly Ser Phe Gly Asn Ile Phe Ser Arg
Asn Ser Ile Asn Asn Ala Leu Met Gly Leu Glu Val Pro Arg Leu Val
Asn Arg Leu Arg Glu Thr Phe Gly Ser Gly Asp Lys Val Leu Thr Arg
Arg Thr Gly Trp Thr Leu Thr Trp Asp Val Arg Lys Ser Gln Ile Glu
Val Gln Glu Gly Pro Gly Gly Pro Lys Trp Thr His Lys Val Gly Glu
Leu Pro Pro Asn Val Gln Glu Ile Ile Ala Lys Gly Gly Leu Glu Lys
Trp Val Lys Asn Ala Ile Gly Ala
SEQ ID NO: 60
PRT - Penicillium chrysogenum
Met Pro Ser Ala Glu Ser Gly Pro Lys Thr Leu Tyr Asp Lys Val Phe
Gln Asp His Ile Val Asn Glu Gln Glu Asp Gly Thr Cys Leu Ile Tyr
Ile Asp Arg His Leu Val His Glu Val Thr Ser Pro Gln Ala Phe Glu
Gly Leu Lys Asn Ala Ser Arg Gln Val Arg Arg Pro Asp Cys Thr Leu
Ala Thr Val Asp His Asn Ile Pro Thr Ser Ser Arg Lys Asn Phe Lys
Asn Ala Ala Asp Phe Ile Lys Glu Asn Asp Ser Arg Leu Gln Cys Thr
Thr Leu Glu Glu Asn Val Lys Asp Phe Gly Leu Thr Tyr Phe Gly Met
Gly Asp Lys Arg Gln Gly Ile Val His Ile Ile Gly Pro Glu Gln Gly
Phe Thr Leu Pro Gly Thr Thr Val Val Cys Gly Asp Ser His Thr Ser
Thr His Gly Ala Phe Gly Ala Leu Ala Phe Gly Ile Gly Thr Ser Glu
Val Glu His Val Leu Ala Thr Gln Thr Leu Ile Thr Arg Arg Ser Lys
Asn Met Arg Ile Gln Val Asp Gly Glu Leu Pro Ala Gly Val Thr Ser
Lys Asp Val Val Leu His Ile Ile Gly Val Ile Gly Thr Ala Gly Gly
Asn Gly Ala Val Ile Glu Phe Cys Gly Ser Val Ile Arg Gly Leu Ser
Met Glu Ala Arg Met Ser Met Cys Asn Met Ser Ile Glu Gly Gly Ala
Arg Ala Gly Met Ile Ala Pro Asp Glu Ile Thr Phe Glu Tyr Leu Lys
Gly Arg Pro Leu Ala Pro Lys Tyr Gly Ser Ala Glu Trp Asn Lys Ala
Thr Ser Tyr Trp Ser Ser Leu Lys Ser Asp Ala Gly Ala Lys Tyr Asp
Ser Glu Val Phe Ile Asp Gly Lys Asp Ile Ile Pro Thr Ile Ser Trp
Gly Thr Ser Pro Gln Asp Val Val Pro Ile Thr Gly Val Val Pro Ser
Pro Asp Asp Phe Glu Asp Glu Asn Arg Lys Ala Ser Cys Lys Arg Ala
Leu Glu Tyr Met Gly Leu Val Ser Gly Thr Pro Met Lys Asp Val Val
Val Asp Lys Val Phe Ile Gly Ser Cys Thr Asn Ala Arg Ile Glu Asp
Leu Arg Ala Ala Ala Lys Val Val Asn Gly Arg Lys Val Ala Ser Asn
Ile Lys Arg Ala Met Ile Val Pro Gly Ser Gly Leu Val Lys Glu Gln
Ala Glu Ser Glu Gly Leu Asp Lys Val Phe Thr Asp Ala Gly Phe Glu
Trp Arg Glu Ala Gly Cys Ser Met Cys Leu Gly Met Asn Pro Asp Ile
Leu Ser Pro Lys Glu Arg Cys Ala Ser Thr Ser Asn Arg Asn Phe Glu
Gly Arg Gln Gly Ala Gln Gly Arg Thr His Leu Met Ser Pro Ala Met
Ala Ala Thr Ala Ala Ile Val Gly Lys Leu Ala Asp Val Arg Glu His
Val Val Ala Ser Pro Val Leu Gly Lys Ala Ser Pro Lys Ile Asp Val
Gln Pro Val Phe Glu Ser Pro Glu Thr Glu Asp Glu Leu Asp Arg Val
Leu Asp Arg Pro Ala Asp Asn Glu Pro His Thr Asn Ser Ser Ala Pro
Ala Ser Gly Gly Gly Lys Ser Thr Gly Leu Pro Thr Phe Thr Thr Leu
Lys Gly Ile Ala Ala Pro Leu Asp Arg Ala Asn Val Asp Thr Asp Ala
Ile Ile Pro Lys Gln Phe Leu Lys Thr Ile Lys Arg Thr Gly Leu Gly
Thr Ala Leu Phe Tyr Glu Leu Arg Tyr Thr Asp Asp Lys Glu Asn Pro
Asp Phe Val Leu Asn Gln Gly Ile Tyr Arg Asp Ser Lys Ile Leu Val
Val Thr Gly Pro Asn Phe Gly Cys Gly Ser Ser Arg Glu His Ala Pro
Trp Ala Leu Leu Asp Phe Gly Ile Lys Cys Ile Ile Ala Pro Ser Phe
Ala Asp Ile Phe Phe Asn Asn Thr Phe Lys Asn Gly Met Leu Pro Val
Val Val Ser Asp Glu Val Ala Leu Gln Lys Ile Ala Asp Glu Ala Arg
Ala Gly Arg Glu Val Glu Val Asp Leu Val Asn Gln Glu Ile Lys Asp
Ala Gln Gly Asn Lys Ile Thr Ser Phe Glu Val Glu Ala Phe Arg Lys
His Cys Leu Ile Asn Gly Leu Asp Asp Ile Gly Leu Thr Leu Gln Met
Glu Ser Lys Ile Arg Ser Phe Glu Ser Lys Arg Thr Leu Asp Thr Pro
Trp Leu Asp Gly Ser Ala Tyr Leu Arg Arg Asp Arg Arg Gly Ala Thr
Met Val Glu Ala Ala Pro Val Pro Lys Thr Asn Arg Gly Asp Val Lys
Asn Glu Pro Leu Glu Trp
SEQ ID NO: 61
PRT - Penicillium chrysogenum
Met Ser Pro Cys Ser Met Leu Leu Lys Arg Val Ala Arg Pro Pro Val
Ser Thr Thr Cys Arg Leu Val Arg Pro Arg Trp Ala Pro Ser Phe Gly
Val Pro Ser Arg Thr Ile His His Pro Leu Arg Ser Val Ser Lys Ser
Leu Ser Thr Arg Ala Leu Ser Thr Thr Ala Pro Ala Arg Val Glu Gly
Phe His Ser Gln His Glu Asn Ala Ser Ile Pro Phe Ser Glu Thr Pro
Ser Glu Lys Arg Thr Pro Gln Thr Leu Thr Glu Lys Ile Val Gln Arg
Tyr Ala Val Gly Leu Pro Glu Gly Lys Leu Val Arg Ser Gly Asp Tyr
Ile Ser Leu Ala Pro Gly Tyr Cys Met Thr His Asp Asn Ser Trp Pro
Val Ala Leu Lys Phe Met Ser Met Gly Ala Thr Lys Ile His Arg Pro
Glu Gln Ile Val Met Thr Leu Asp His Asp Val Gln Asn Thr Ser Ala
Ala Asn Leu Lys Lys Tyr Glu Gln Ile Glu Thr Phe Ala Gly Gln His
Gly Ile Asp Phe Tyr Pro Ala Gly Arg Gly Ile Gly His Gln Val Met
Val Glu Glu Gly Tyr Ala Trp Pro Gly Thr Met Ala Val Ala Ser Asp
Ser His Ser Asn His Tyr Gly Gly Val Gly Cys Leu Gly Thr Ala Val
Val Arg Thr Asp Ala Ala Ser Ile Trp Ala Thr Ser Arg Thr Trp Trp
Gln Ile Pro Pro Val Ala Arg Val Thr Phe Thr Gly Thr Leu Pro Ala
Gly Val Thr Gly Lys Asp Val Ile Val Ala Leu Cys Gly Leu Phe Asn
Ser Asp Val Leu Asn His Ala Ile Glu Phe Thr Gly Ser Glu Glu Thr
Met Glu Ser Leu Leu Val Asp Ser Arg Leu Thr Ile Ala Asn Met Thr
Thr Glu Trp Gly Ala Leu Thr Gly Leu Phe Pro Ile Asp Arg Thr Leu
Lys Arg Trp Leu Arg Tyr Lys Ala Thr Glu Ala Ala Met Ser Glu Asp
Arg Thr Thr Arg Gln Arg Ile Thr His Glu Arg Ile Asp Glu Leu Phe
Ala Asn Pro Leu Thr Ala Asp Pro Asp Ala Gln Tyr Ala Lys Gln Leu
Tyr Leu Asn Leu Ser Thr Leu Ser Pro Tyr Val Ser Gly Pro Asn Ser
Val Lys Val Ala Thr Pro Leu Asn Glu Leu Ala Gln Gln Asn Ile Lys
Val Asn Arg Ala Tyr Ile Val Ser Cys Thr Asn Ser Arg Ala Ser Asp
Leu Ala Ala Ala Ala Lys Val Phe Lys Asp Ala Ala Lys Ala Asn Pro
Gly Thr Thr Pro Lys Ile Ala Asp Gly Val Lys Leu Tyr Ile Ala Ala
Ala Ser Ala Pro Glu Gln Glu Ala Ala Glu Ser Thr Gly Asp Trp Gln
Ala Leu Leu Asp Ala Gly Ala Gln Pro Leu Pro Ala Gly Cys Gly Pro
Cys Ile Gly Leu Gly Thr Gly Leu Leu Glu Pro Gly Glu Val Gly Ile
Ser Ala Ser Asn Arg Asn Phe Lys Gly Arg Met Gly Ser Arg Asp Ala
Leu Ala Tyr Leu Ala Ser Pro Glu Val Val Ala Ala Ser Ala Leu Ser
Gly Val Ile Ser Gly Pro Gly Ala Tyr Gln Val Pro Glu Asn Trp Ser
Gly Val Glu His Gly Phe Gly Thr Gly Leu Pro Pro Thr Thr Glu Asn
Glu Leu Thr Asn Leu Leu Gln Gln Met Glu Ser Leu Ile Asp Arg Val
Glu Ser Ala Gly Glu Asp Ser Lys Pro Ala Thr Glu Ile Leu Pro Gly
Phe Pro Glu Arg Ile Ser Gly Glu Ile Val Phe Leu Asp Ala Asp Asn
Leu Asp Thr Asp Asn Ile Tyr Pro Gly Lys Leu Thr Tyr Gln Asp Asn
Val Ser Lys Asp Asp Met Ala Ala Ala Cys Met Gln Asn Tyr Asp Pro
Glu Phe Lys Gly Ile Ala Lys Pro Ser Asp Ile Leu Val Ala Gly Phe
Asn Phe Gly Cys Gly Ser Ser Arg Glu Gln Ala Ala Thr Ala Ile Leu
Ala Lys Gln Ile Pro Leu Val Val Ala Gly Ser Phe Gly Asn Ile Phe
Ser Arg Asn Ser Ile Asn Asn Ala Leu Met Gly Leu Glu Val Pro Arg
Leu Ile Glu Arg Leu Arg Ala Ser Phe Ala Gln Pro Pro Pro Gly Asp
Ala Gly Arg Gln Leu Thr Arg Arg Thr Gly Trp Thr Leu Thr Trp Asp
Val Lys Arg Ser Val Val Glu Val Lys Glu Gly Glu Ser Gly Glu Ser
Trp Thr Glu Gln Val Gly Glu Leu Pro Ala Asn Val Gln Glu Ile Ile
Ala Glu Gly Gly Leu Glu Ala Trp Val Lys Gly Lys Val Ala Lys Ser
Glu
SEQ ID NO: 62
PRT - Phanerochaete chrysosporium
Met Ala Phe Arg Leu Pro Leu Arg Arg Ala Leu Ser Thr Ala Ala Ala
Ser Arg Ser Ser Leu Lys Ile Gly Leu Val Pro Ala Asp Gly Ile Gly
Arg Glu Val Ile Pro Ala Ala Arg Gln Ala Ile Glu Ala Leu Gly Ser
Asp Ile Pro Lys Pro Glu Phe Val Asp Leu Leu Ala Gly Phe Glu Leu
Phe Thr Arg Thr Gly Thr Ala Leu Pro Glu Glu Thr Val Gln Ala Leu
Lys Glu Cys Asp Cys Ala Leu Phe Gly Ala Val Ser Ser Pro Ser Arg
Arg Val Thr Gly Tyr Ser Ser Pro Ile Val Ala Leu Arg Lys Ile Leu
Asp Leu Tyr Ala Asn Val Arg Pro Val Val Ala Pro Thr Pro Glu Glu
Lys Pro Asn Val Asp Leu Ile Val Val Arg Glu Asn Thr Glu Cys Leu
Tyr Val Lys Gln Glu Gln Met Thr Pro Thr Glu Asn Gly Arg Glu Ala
Arg Ala Thr Arg Val Ile Thr Glu Arg Ala Ser Arg Arg Ile Gly Gln
Met Ala Phe Glu Leu Ala Lys Ala Arg Pro Arg Lys His Val Thr Ile
Ile His Lys Ser Asn Val Leu Ser Ile Thr Asp Gly Leu Phe Arg Glu
Thr Val Arg Ser Val Pro Arg Leu Asn Glu Gly Lys Tyr Asp Asp Val
Glu Ile Ala Glu Gln Leu Val Asp Ser Ala Val Tyr Arg Leu Phe Arg
Glu Pro His Ile Tyr Asp Val Met Val Ala Pro Asn Leu Tyr Gly Asp
Ile Ile Ser Asp Ala Ala Ala Ala Leu Val Gly Ser Leu Gly Leu Val
Pro Ser Val Asn Ala Gly Asp Asn Phe Val Met Gly Glu Pro Val His
Gly Ser Ala Pro Asp Ile Ala Gly Gln Gly Ile Ala Asn Pro Ile Ala
Ser Ile Arg Ser Ala Ala Leu Met Leu Arg His Leu Gly Tyr Gly Ala
Pro Ala Asp Arg Leu Asp Lys Ala Val Asp Glu Val Ile Arg Glu Gly
Gln Ile Leu Thr Pro Asp Leu Gly Gly Lys Ser Lys Thr Gln Asp Val
Val Asp Ala Val Leu Lys Arg Ile
SEQ ID NO: 63
PRT - Schizosaccharomyces pombe
Met Ser Ala Thr Arg Arg Ile Val Leu Gly Leu Ile Pro Ala Asp Gly
Ile Gly Lys Glu Val Val Pro Ala Ala Arg Arg Leu Met Glu Asn Leu
Pro Ala Lys His Lys Leu Lys Phe Asp Phe Ile Asp Leu Asp Ala Gly
Trp Gly Thr Phe Glu Arg Thr Gly Lys Ala Leu Pro Glu Arg Thr Val
Glu Arg Leu Lys Thr Glu Cys Asn Ala Ala Leu Phe Gly Ala Val Gln
Ser Pro Thr His Lys Val Ala Gly Tyr Ser Ser Pro Ile Val Ala Leu
Arg Lys Lys Met Gly Leu Tyr Ala Asn Val Arg Pro Val Lys Ser Leu
Asp Gly Ala Lys Gly Lys Pro Val Asp Leu Val Ile Val Arg Glu Asn
Thr Glu Cys Leu Tyr Val Lys Glu Glu Arg Met Val Gln Asn Thr Pro
Gly Lys Arg Val Ala Glu Ala Ile Arg Arg Ile Ser Glu Glu Ala Ser
Thr Lys Ile Gly Lys Met Ala Phe Glu Ile Ala Lys Ser Arg Gln Lys
Ile Arg Glu Ser Gly Thr Tyr Ser Ile His Lys Lys Pro Leu Val Thr
Ile Ile His Lys Ser Asn Val Met Ser Val Thr Asp Gly Leu Phe Arg
Glu Ser Cys Arg His Ala Gln Ser Leu Asp Pro Ser Tyr Ala Ser Ile
Asn Val Asp Glu Gln Ile Val Asp Ser Met Val Tyr Arg Leu Phe Arg
Glu Pro Glu Cys Phe Asp Val Val Val Ala Pro Asn Leu Tyr Gly Asp
Ile Leu Ser Asp Gly Ala Ala Ser Leu Ile Gly Ser Leu Gly Leu Val
Pro Ser Ala Asn Val Gly Asp Asn Phe Val Met Ser Glu Pro Val His
Gly Ser Ala Pro Asp Ile Ala Gly Arg Gly Ile Ala Asn Pro Val Ala
Thr Phe Arg Ser Val Ala Leu Met Leu Glu Phe Met Gly His Gln Asp
Ala Ala Ala Asp Ile Tyr Thr Ala Val Asp Lys Val Leu Thr Glu Gly
Lys Val Leu Thr Pro Asp Leu Gly Gly Lys Ser Gly Thr Asn Glu Ile
Thr Asp Ala Val Leu Ala Asn Ile His Asn
SEQ ID NO: 64
PRT - Emericella nidulans
Met Ala Ala Ala Arg Thr Leu Arg Ile Gly Leu Ile Pro Gly Asp Gly
Ile Gly Arg Glu Val Ile Pro Ala Gly Arg Arg Ile Leu Glu Ala Leu
Pro Ala Ser Leu Asn Leu Lys Phe Asn Phe Val Asp Leu Asp Ala Gly
Tyr Asp Cys Phe Lys Arg Thr Gly Thr Ala Leu Pro Asp Lys Thr Val
Glu Val Leu Lys Lys Glu Cys Asp Gly Ala Leu Phe Gly Ala Val Ser
Ser Pro Ser Thr Lys Val Ala Gly Tyr Ser Ser Pro Ile Val Ala Leu
Arg Lys Lys Leu Asp Leu Phe Ala Asn Val Arg Pro Val Lys Thr Thr
Ala Gly Thr Ser Ala Gly Lys Pro Ile Asp Leu Val Ile Val Arg Glu
Asn Thr Glu Asp Leu Tyr Val Lys Glu Glu Ser Thr Glu Glu Thr Pro
Asn Gly Lys Val Ala Arg Ala Ile Lys Gln Ile Ser Glu Arg Ala Ser
Ser Arg Ile Ala Thr Ile Ala Gly Glu Ile Ala Leu Arg Arg Gln Asn
Ile Arg Asp Gly Ala Ala Ala Ser Gly Leu Arg Thr Lys Pro Met Val
Thr Ile Thr His Lys Ser Asn Val Leu Ser Gln Thr Asp Gly Leu Phe
Arg Glu Thr Ala Arg Ala Ala Leu Ala Ala Gln Lys Phe Ser Ser Val
Glu Val Glu Glu Gln Ile Val Asp Ser Met Val Tyr Lys Leu Phe Arg
Gln Pro Glu Tyr Tyr Asp Val Ile Val Ala Pro Asn Leu Tyr Gly Asp
Ile Leu Ser Asp Gly Ala Ala Ala Leu Val Gly Ser Leu Gly Leu Val
Pro Ser Ala Asn Val Gly Asp Asn Phe Ala Ile Gly Glu Pro Cys His
Gly Ser Ala Pro Asp Ile Glu Gly Lys Asn Ile Ala Asn Pro Ile Ala
Thr Leu Arg Ser Val Ala Leu Met Leu Glu Phe Leu Gly Glu Glu Gln
Ala Ala Ala Lys Ile Tyr Ala Ala Val Asp Gly Asn Leu Asp Glu Gly
Lys Tyr Leu Ser Pro Asp Met Gly Gly Lys Ala Thr Thr Thr Glu Val
Leu Glu Asp Val Leu Lys Arg Leu
SEQ ID NO: 65
PRT - Penicillium chrysogenum
Met Ala Ala Ala Arg Thr Leu Arg Ile Gly Leu Ile Pro Gly Asp Gly
Ile Gly Arg Glu Val Ile Pro Ala Gly Arg Arg Ile Leu Glu Ser Leu
Pro Ser Ser Leu Asn Leu Lys Phe Ser Phe Val Asp Leu Asp Ala Gly
Tyr Glu Thr Phe Gln Lys Thr Gly Thr Ala Leu Pro Asp Lys Thr Val
Asp Thr Leu Lys Lys Glu Cys Asp Gly Ala Leu Phe Gly Ala Val Ser
Ser Pro Ser Thr Lys Val Ala Gly Tyr Ser Ser Pro Ile Val Ala Leu
Arg Lys Lys Leu Asp Leu Tyr Ala Asn Val Arg Pro Val Lys Thr Thr
Ala Gly Asn Ser Asn Gly Lys Pro Ile Asp Leu Val Ile Val Arg Glu
Asn Thr Glu Asp Leu Tyr Val Lys Glu Glu Arg Thr Ile Glu Gly Pro
Asn Gly Lys Val Ala Glu Ala Ile Lys Arg Ile Ser Glu Lys Ala Ser
Phe Arg Ile Ser Asn Ile Ala Gly Glu Ile Ala Leu Arg Arg Gln Asn
Ile Arg Ala Ala Ser Pro Thr Ser Thr Arg Asp Gln Pro Met Val Thr
Ile Thr His Lys Ser Asn Val Leu Ser Gln Thr Asp Gly Leu Phe Arg
Glu Thr Ala Arg Arg Ala Leu Ser Ala Glu Lys Phe Ser Ser Val Phe
Val Glu Glu Gln Ile Val Asp Ser Met Val Tyr Lys Leu Phe Arg Gln
Pro Glu Phe Tyr Asp Val Ile Val Ala Pro Asn Leu Tyr Gly Asp Ile
Leu Ser Asp Gly Ala Ala Ala Leu Val Gly Ser Leu Gly Leu Val Pro
Ser Ala Asn Val Gly Asp Gly Phe Ala Ile Gly Glu Pro Cys His Gly
Ser Ala Pro Asp Ile Glu Gly Lys Gly Ile Ser Asn Pro Ile Ala Thr
Ile Arg Ser Val Ala Leu Met Leu Glu Phe Leu Gly Glu Glu Lys Ala
Ala Ala Gln Ile Tyr Ala Ala Val Asp Gly Asn Leu Asp Ala Ala Gln
Phe Leu Thr Pro Asp Met Gly Gly Lys Ala Thr Thr Gln Gln Val Leu
Asp Asp Val Leu Lys Arg Leu
SEQ ID NO: 66
PRT - Saccharomyces cerevisiae
Met Phe Arg Ser Val Ala Thr Arg Leu Ser Ala Cys Arg Gly Leu Ala
Ser Asn Ala Ala Arg Lys Ser Leu Thr Ile Gly Leu Ile Pro Gly Asp
Gly Ile Gly Lys Glu Val Ile Pro Ala Gly Lys Gln Val Leu Glu Asn
Leu Asn Ser Lys His Gly Leu Ser Phe Asn Phe Ile Asp Leu Tyr Ala
Gly Phe Gln Thr Phe Gln Glu Thr Gly Lys Ala Leu Pro Asp Glu Thr
Val Lys Val Leu Lys Glu Gln Cys Gln Gly Ala Leu Phe Gly Ala Val
Gln Ser Pro Thr Thr Lys Val Glu Gly Tyr Ser Ser Pro Ile Val Ala
Leu Arg Arg Glu Met Gly Leu Phe Ala Asn Val Arg Pro Val Lys Ser
Val Glu Gly Glu Lys Gly Lys Pro Ile Asp Met Val Ile Val Arg Glu
Asn Thr Glu Asp Leu Tyr Ile Lys Ile Glu Lys Thr Tyr Ile Asp Lys
Ala Thr Gly Thr Arg Val Ala Asp Ala Thr Lys Arg Ile Ser Glu Ile
Ala Thr Arg Arg Ile Ala Thr Ile Ala Leu Asp Ile Ala Leu Lys Arg
Leu Gln Thr Arg Gly Gln Ala Thr Leu Thr Val Thr His Lys Ser Asn
Val Leu Ser Gln Ser Asp Gly Leu Phe Arg Glu Ile Cys Lys Glu Val
Tyr Glu Ser Asn Lys Asp Lys Tyr Gly Gln Ile Lys Tyr Asn Glu Gln
Ile Val Asp Ser Met Val Tyr Arg Leu Phe Arg Glu Pro Gln Cys Phe
Asp Val Ile Val Ala Pro Asn Leu Tyr Gly Asp Ile Leu Ser Asp Gly
Ala Ala Ala Leu Val Gly Ser Leu Gly Val Val Pro Ser Ala Asn Val
Gly Pro Glu Ile Val Ile Gly Glu Pro Cys His Gly Ser Ala Pro Asp
Ile Ala Gly Lys Gly Ile Ala Asn Pro Ile Ala Thr Ile Arg Ser Thr
Ala Leu Met Leu Glu Phe Leu Gly His Asn Glu Ala Ala Gln Asp Ile
Tyr Lys Ala Val Asp Ala Asn Leu Arg Glu Gly Ser Ile Lys Thr Pro
Asp Leu Gly Gly Lys Ala Ser Thr Gln Gln Val Val Asp Asp Val Leu
Ser Arg Leu
SEQ ID NO: 67
PRT - Kluyveromyces lactis
Met Met Arg Thr Arg Phe Ile Gln Leu Ser Arg Arg Ala Tyr Ala Ser
Asn Ala Lys Asn Leu Thr Ile Gly Leu Ile Pro Gly Asp Gly Ile Gly
Lys Glu Val Ile Pro Ala Gly Lys Lys Ile Leu Glu Ser Leu Asn Pro
Lys Tyr Gly Leu Ser Phe Lys Phe Ile Asp Leu Gln Ala Gly Trp Glu
Thr Phe Gln Asn Thr Gly Lys Ala Leu Pro Asp Glu Thr Ile Asp Ile
Leu Lys Asn Gln Cys Glu Gly Ala Leu Phe Gly Ala Val Gln Ser Pro
Thr Thr Lys Val Glu Gly Tyr Ser Ser Pro Ile Val Ala Leu Arg Lys
Asn Leu Gly Leu Phe Ala Asn Val Arg Pro Val Lys Ser Val Asp Gly
Thr Lys Asp Arg Lys Val Asp Leu Val Ile Val Arg Glu Asn Thr Glu
Asp Leu Tyr Ile Lys Leu Glu Lys Ser Tyr Ile Asp Glu Ala Thr Gly
Thr Arg Val Ala Asp Ala Thr Lys Arg Ile Thr Glu Ile Ala Thr Lys
Asn Ile Ala Thr Ile Ala Leu Gln Ile Ala Gln Gln Arg Leu Glu Gln
Asn Gly His Ala Thr Leu Thr Val Thr His Lys Ser Asn Val Leu Ser
Gln Ser Asp Gly Leu Phe Arg Glu Val Cys Arg Glu Thr Tyr Glu Ala
Asn Lys Asp Lys Tyr Gly Gly Val Gln Tyr Asn Glu Gln Ile Val Asp
Ser Met Val Tyr Arg Met Phe Arg Glu Pro Glu Cys Phe Asp Val Val
Val Ala Pro Asn Leu Tyr Gly Asp Ile Leu Ser Asp Gly Ala Ala Ala
Leu Val Gly Ser Leu Gly Val Val Pro Ser Ala Asn Val Gly Pro Asn
Ile Val Ile Gly Glu Pro Cys His Gly Ser Ala Pro Asp Ile Ala Gly
Lys Gly Ile Ser Asn Pro Ile Ala Thr Ile Arg Ser Thr Ala Leu Met
Leu Glu Phe Leu Gly Tyr Pro Glu Pro Ala Lys Asp Ile His Lys Ala
Val Asp Ala Asn Ile Arg Glu Gly Lys Tyr Leu Thr Pro Asp Leu Gly
Gly Asn Ser Thr Thr Gln Gln Val Leu Glu Asp Val Leu Ser Lys Leu
Asp
SEQ ID NO: 68
PRT - Penicillium chrysogenum
Met Ser Pro Pro Thr Ala Leu Asp Val Asn Leu Val Gly Val Thr Asp
Thr Ser Thr Val Pro Val Pro Glu Pro Leu Thr Val Asn Gly Val Ser
Ala Trp Arg Glu Lys Thr Ala Lys Val Pro Thr Gly Val Ala Ala Ala
Cys Asn Ser Asp Met Phe Lys Ser Pro Ile Cys Tyr Thr Lys Pro Lys
Ala Lys Gln Phe Glu His Arg Phe Ser Leu Glu Ala Lys Ser Arg Lys
Ala Ser Thr Leu Lys Thr Ala Ala Arg Tyr Leu Lys Thr Pro Gly Leu
Ile Ser Leu Gly Gly Gly Leu Pro Ser Pro Glu Tyr Phe Pro Phe Glu
His Leu Asp Ile Lys Val Pro Thr Ala Pro Gly Phe Ser Pro Glu Ala
Thr Arg Glu Ser Gly Thr Val Leu Arg Ala Gly Lys His Asp Ile Gln
Glu Gly Thr Ser Thr Tyr Asp Leu Glu Ile Ala Leu Asn Tyr Gly Gln
Ala Thr Gly Ala Ala Pro Leu Leu Arg Phe Val Thr Glu His Thr Glu
Ile Ile His Ser Pro Pro Tyr Ser Asp Trp Gln Cys Thr Leu Thr Ala
Gly Ser Thr Tyr Ala Trp Asp Thr Ala Leu Arg Val Phe Cys Glu Arg
Gly Asp Tyr Ile Leu Met Glu Glu Tyr Thr Phe Ala Ser Ala Ala Glu
Thr Ala Phe Pro Leu Gly Ile Lys Val Ala Gly Ile Pro Met Asp Glu
Gln Gly Leu Ile Pro Glu Ala Met Asp Lys Ile Leu Gly Asp Trp Asp
Val Ala Ala Arg Gly Ala Arg Lys Pro His Val Leu Tyr Thr Ile Pro
Thr Gly Gln Asn Pro Thr Gly Ala Thr Gln Ser Ala Glu Arg Arg His
Ala Val Tyr Lys Val Ala Gln Lys His Asp Leu Ile Ile Val Glu Asp
Glu Pro Tyr Tyr Phe Leu Gln Met Gln Pro Tyr Thr Ser Gly Asp Ala
Ser Pro Val Pro Pro Pro Ser Ser His Glu Glu Phe Ile Asn Ser Leu
Val Pro Ser Phe Leu Ser Met Asp Thr Asp Gly Arg Val Val Arg Leu
Glu Ser Phe Ser Lys Val Ile Ser Pro Gly Ser Arg Val Gly Trp Ile
Val Ala Ser Glu Gln Ile Ile Glu Arg Phe Ile Arg Asn Phe Glu Val
Ser Ser Gln Asn Pro Ser Gly Ile Ala Gln Ile Ala Leu Phe Lys Leu
Leu Asp Glu His Trp Gly His Ser Gly Tyr Leu Asp Trp Leu Ile Asn
Leu Arg Met Ser Tyr Thr Ala Arg Arg Asp Ser Leu Val His Ala Cys
Glu Lys His Leu Pro Arg Glu Ile Val His Trp Glu Ala Pro Ala Ala
Gly Met Phe Gln Trp Met Ser Ile Asp Trp Arg Lys His Pro Gly Ile
Ala Ala Gly Lys Thr His Ala Asp Ile Glu Glu Glu Ile Phe Leu Ser
Ala Val Asn Gly Gly Val Leu Leu Ser Arg Gly Ser Trp Phe Lys Pro
Asp His Asp Thr Val Glu Glu Lys Met Phe Phe Arg Ala Thr Phe Ala
Ala Ala Ser Ser Glu Lys Ile Asp Glu Ala Ile Ser Arg Phe Ala Gln
Ser Leu Arg Ala Gln Phe Gly Leu
SEQ ID NO: 69
PRT - Thermus thermophilus
Met Arg Glu Trp Lys Ile Ile Asp Ser Thr Leu Arg Glu Gly Glu Gln
Phe Glu Lys Ala Asn Phe Ser Thr Gln Asp Lys Val Glu Ile Ala Lys
Ala Leu Asp Glu Phe Gly Ile Glu Tyr Ile Glu Val Thr Thr Pro Val
Ala Ser Pro Gln Ser Arg Lys Asp Ala Glu Val Leu Ala Ser Leu Gly
Leu Lys Ala Lys Val Val Thr His Ile Gln Cys Arg Leu Asp Ala Ala
Lys Val Ala Val Glu Thr Gly Val Gln Gly Ile Asp Leu Leu Phe Gly
Thr Ser Lys Tyr Leu Arg Ala Ala His Gly Arg Asp Ile Pro Arg Ile
Ile Glu Glu Ala Lys Glu Val Ile Ala Tyr Ile Arg Glu Ala Ala Pro
His Val Glu Val Arg Phe Ser Ala Glu Asp Thr Phe Arg Ser Glu Glu
Gln Asp Leu Leu Ala Val Tyr Glu Ala Val Ala Pro Tyr Val Asp Arg
Val Gly Leu Ala Asp Thr Val Gly Val Ala Thr Pro Arg Gln Val Tyr
Ala Leu Val Arg Glu Val Arg Arg Val Val Gly Pro Arg Val Asp Ile
Glu Phe His Gly His Asn Asp Thr Gly Cys Ala Ile Ala Asn Ala Tyr
Glu Ala Ile Glu Ala Gly Ala Thr His Val Asp Thr Thr Ile Leu Gly
Ile Gly Glu Arg Asn Gly Ile Thr Pro Leu Gly Gly Phe Leu Ala Arg
Met Tyr Thr Leu Gln Pro Glu Tyr Val Arg Arg Lys Tyr Lys Leu Glu
Met Leu Pro Glu Leu Asp Arg Met Val Ala Arg Met Val Gly Val Glu
Ile Pro Phe Asn Asn Tyr Ile Thr Gly Glu Thr Ala Phe Ser His Lys
Ala Gly Met His Leu Lys Ala Ile Tyr Ile Asn Pro Glu Ala Tyr Glu
Pro Tyr Pro Pro Glu Val Phe Gly Val Lys Arg Lys Leu Ile Ile Ala
Ser Arg Leu Thr Gly Arg His Ala Ile Lys Ala Arg Ala Glu Glu Leu
Gly Leu His Tyr Gly Glu Glu Glu Leu His Arg Val Thr Gln His Ile
Lys Ala Leu Ala Asp Arg Gly Gln Leu Thr Leu Glu Glu Leu Asp Arg
Ile Leu Arg Glu Trp Ile Thr Ala
SEQ ID NO: 70
PRT - Deinococcus radiourans
Met Ala Gly Ile Phe Met Thr Asp Ala Pro Pro Pro Leu Ile Pro Ala
Arg Ser Trp Ala Ile Ile Asp Ser Thr Leu Arg Glu Gly Glu Gln Phe
Ala Arg Gly Asn Phe Gly Thr Asp Asp Lys Val Glu Ile Ala Arg Ala
Leu Asp Ala Phe Gly Ala Glu Tyr Ile Glu Val Thr Thr Pro Met Val
Ser Glu Gln Thr Arg Gln Asp Ile Arg Lys Leu Thr Gly Leu Gly Leu
Arg Ala Lys Phe Leu Thr His Val Arg Cys His Met Glu Asp Val Gln
Arg Ala Val Asp Thr Gly Val Asp Gly Leu Asp Leu Leu Phe Gly Thr
Ser Ser Phe Leu Arg Glu Phe Ser His Gly Lys Ser Ile Ala Gln Ile
Ile Asp Thr Ala Gly Glu Val Ile Gly Trp Ile Lys Thr His His Pro
Glu Leu Glu Ile Arg Phe Ser Ala Glu Asp Thr Phe Arg Ser Glu Glu
Ala Asp Leu Met Ala Val Tyr Ser Ala Val Ser Glu Leu Gly Val His
Arg Val Gly Leu Ala Asp Thr Val Gly Val Ala Thr Pro Arg Gln Val
Tyr Thr Leu Val Arg Glu Val Arg Lys Val Ile His Glu Gly Cys Gly
Ile Glu Phe His Gly His Asn Asp Thr Gly Cys Ala Val Ser Asn Ala
Tyr Glu Ala Ile Glu Ala Gly Ala Thr His Ile Asp Thr Thr Ile Leu
Gly Ile Gly Glu Arg Asn Gly Ile Thr Pro Leu Gly Gly Leu Leu Ala
Arg Met Phe Thr Phe Asp Pro Gln Gly Leu Ile Asp Lys Tyr Asn Leu
Glu Leu Leu Pro Glu Leu Asp Arg Met Ile Ala Arg Met Val Asp Leu
Pro Val Pro Trp Asn Asn Tyr Leu Thr Gly Glu Phe Ala Tyr Asn His
Lys Ala Gly Met His Leu Lys Ala Ile Tyr Leu Asn Pro Gly Ala Tyr
Glu Ala Ile Pro Pro Gly Val Phe Gly Val Gly Arg Arg Ile Gln Ala
Ala Ser Lys Val Thr Gly Lys His Ala Ile Ala Tyr Lys Ala Arg Glu
Leu Gly Leu His Tyr Gly Glu Asp Ala Leu Arg Arg Val Thr Asp His
Ile Lys Ser Leu Ala Glu Gln Asp Glu Leu Asp Asp Ala His Leu Glu
Gln Val Leu Arg Glu Trp Val Ser Ala
SEQ ID NO: 71
PRT - Deinococcus geothermalis
Met Thr Pro Asp Ser Ser Thr Pro Leu Ile Pro Ala Arg Ser Trp Ala
Ile Ile Asp Ser Thr Leu Arg Glu Gly Glu Gln Phe Ala Arg Gly Asn
Phe Lys Thr Gly Asp Lys Ile Glu Ile Ala Arg Leu Leu Asp Ala Phe
Gly Ala Glu Phe Leu Glu Val Thr Thr Pro Met Val Gly Ala Gln Thr
Gln Ala Asp Ile Arg Arg Leu Thr Ser Leu Gly Leu Asn Ala Lys Ile
Leu Thr His Val Arg Cys His Leu Glu Asp Val Gln Arg Ala Val Asp
Leu Gly Val Asp Gly Leu Asp Leu Leu Phe Gly Thr Ser Ser Phe Leu
Arg Glu Phe Ser His Gly Lys Ser Ile Ala Gln Ile Ile Asp Thr Ala
Ser Glu Val Ile Gly Trp Ile Lys Gln Asn His Pro Asp Leu Glu Ile
Arg Phe Ser Ala Glu Asp Thr Phe Arg Ser Glu Glu Ala Asp Leu Met
Ala Val Tyr Arg Ala Val Ser Asp Leu Gly Val His Arg Val Gly Leu
Ala Asp Thr Val Gly Val Ala Thr Pro Arg Gln Val Tyr Thr Leu Val
Arg Glu Val Arg Lys Val Ile His Ala Glu Cys Gly Ile Glu Phe His
Gly His Asn Asp Thr Gly Cys Ala Val Ser Asn Ala Tyr Glu Ala Ile
Glu Ala Gly Ala Thr His Ile Asp Thr Thr Ile Leu Gly Ile Gly Glu
Arg Asn Gly Ile Thr Pro Leu Gly Gly Phe Leu Ala Arg Met Phe Thr
Phe Asp Pro Gln Gly Leu Ile Asp Lys Tyr Asn Leu Glu Leu Leu Pro
Glu Leu Asp Arg Leu Ile Ala Arg Leu Val Asp Leu Pro Ile Pro Trp
Asn Asn Tyr Leu Thr Gly Glu Phe Ala Tyr Asn His Lys Ala Gly Met
His Leu Lys Ala Ile Tyr Leu Asn Pro Gly Ala Tyr Glu Ala Ile Pro
Pro Ser Val Phe Gly Val Gly Arg Arg Ile Gln Ala Ala Ser Lys Val
Thr Gly Lys His Ala Ile Ala His Lys Ala Arg Glu Leu Gly Leu His
Tyr Gly Glu Asp Ala Leu Arg Arg Val Thr Asp His Ile Lys Ala Leu
Ala Glu Glu Gly Glu Leu Asp Asp Ala His Leu Glu Gln Val Leu Arg
Glu Trp Val Arg Ala
SEQ ID NO: 72
PRT - Sulfolobus solfataricus
Met Ala Leu Lys Met Lys Tyr Asp Phe Leu Leu Leu Ser Leu Lys Leu
Leu Asn Leu Pro Ile Ile Phe His Leu Cys Ser Val Ser Lys Lys Ser
Val Glu Val Leu Asp Thr Thr Leu Arg Asp Gly Ser Gln Gly Ala Asn
Ile Ser Phe Thr Leu Asn Asp Lys Ile Lys Ile Ala Leu Leu Leu Asp
Glu Leu Gly Val Asp Tyr Ile Glu Gly Gly Trp Pro Gly Ser Asn Pro
Lys Asp Glu Glu Phe Phe Arg Glu Ile Lys Lys Tyr Arg Leu Ser Lys
Ala Lys Ile Ala Ala Phe Gly Ser Thr Lys Arg Lys Asp Val Ser Val
Lys Glu Asp Ile Ser Leu Asn Ser Ile Val Lys Ala Asp Val Asp Val
Ala Val Ile Phe Gly Lys Ser Trp Ser Leu His Ala Thr Glu Val Leu
Lys Val Thr Lys Gln Asp Asn Leu Asp Ile Val Tyr Asp Ser Ile Asn
Tyr Leu Lys Ser His Gly Leu Lys Val Ile Phe Asp Ala Glu His Phe
Tyr Gln Gly Phe Lys Glu Asp Pro Glu Tyr Ala Leu Glu Val Val Lys
Thr Ala Glu Ser Ala Gly Ala Asp Val Ile Ala Leu Ala Asp Thr Asn
Gly Gly Thr Pro Pro Phe Glu Val Tyr Glu Ile Thr Lys Lys Val Arg
Glu Val Leu Gln Val Lys Leu Gly Ile His Ala His Asn Asp Ile Gly
Cys Ala Val Ala Asn Ser Leu Met Ala Ile Lys Ala Gly Ala Arg His
Val Gln Gly Thr Ile Asn Gly Ile Gly Glu Arg Thr Gly Asn Ala Asp
Leu Ile Gln Ile Ile Pro Thr Leu Ile Leu Lys Met Gly Leu Asn Ala
Leu Asn Gly Gln Glu Ser Leu Arg Lys Leu Arg Glu Val Ser Arg Ile
Val Tyr Glu Ile Leu Gly Leu Pro Pro Asn Pro Tyr Gln Pro Tyr Val
Gly Asp Asn Ala Phe Ala His Lys Ala Gly Val His Val Asp Ala Val
Met Lys Val Pro Arg Ala Tyr Glu His Val Asp Pro Ser Leu Val Gly
Asn Asp Arg Lys Phe Val Ile Ser Glu Leu Ser Gly Thr Ala Asn Leu
Val Ser Tyr Leu Gln Gly Leu Gly Ile Ala Val Asp Lys Lys Asp Glu
Arg Leu Lys Lys Ala Leu Asn Lys Ile Lys Glu Leu Glu Ala Arg Gly
Tyr Ser Phe Asp Val Gly Pro Ala Ser Ala Ile Leu Ile Thr Leu Lys
Glu Leu Asn Ile Tyr Lys Asn Tyr Ile Asn Leu Glu Tyr Trp Lys Val
Ile Asn Glu Asn Asn Gly Leu Ser Ile Gly Ile Val Lys Val Asn Ser
Gln Leu Glu Val Ala Glu Gly Val Gly Pro Val Asn Ala Ile Asp Arg
Ala Leu Arg Met Ala Leu Gln Arg Val Tyr Pro Glu Ile Gly Glu Val
Lys Leu Ile Asp Tyr Arg Val Ile Leu Pro Ser Glu Ile Lys Asn Thr
Glu Ser Val Val Arg Val Thr Ile Glu Phe Thr Asp Asn Lys Met Asn
Trp Arg Thr Glu Gly Val Ser Lys Ser Val Val Glu Ala Ser Val Met
Ala Leu Val Asp Gly Leu Asp Tyr Tyr Leu Gln Leu Lys Lys Thr Leu
Lys Thr Ala Val Asp Asn Tyr Ile Val
SEQ ID NO: 73
PRT - Thermococcus kodakarensis
Met Val Leu Asp Ser Thr Leu Arg Glu Gly Glu Gln Thr Pro Gly Val
Asn Phe Ser Pro Glu Asp Arg Leu Arg Ile Gly Ile Ala Leu Asp Glu
Val Gly Val Asp Phe Ile Glu Ala Gly His Pro Ala Val Ser Gly Glu
Ile Leu Glu Gly Ile Arg Leu Leu Ala Ser His Gly Leu Asn Ala Asn
Ile Leu Ala His Ser Arg Ala Leu Arg Ser Asp Ile Asp Leu Val Leu
Lys Ala Glu Ala Glu Trp Ile Gly Ile Phe Met Cys Leu Ser Gln Arg
Cys Leu Glu Arg Arg Phe Arg Thr Asp Leu Ser Gly Ala Leu Thr Arg
Val Glu Asp Ala Ile Leu Tyr Ala Lys Asp His Gly Leu Lys Ile Arg
Phe Thr Pro Glu Asp Thr Thr Arg Thr Glu Trp Lys Asn Leu Thr Ala
Ala Leu Asn Leu Ala Arg Glu Leu Lys Val Asp Arg Val Ser Ile Ala
Asp Thr Thr Gly Ala Ala His Pro Leu Glu Phe Tyr Asp Leu Val Lys
Arg Val Val Glu Phe Gly Ile Pro Val Asn Val His Cys His Asn Asp
Leu Gly Leu Ala Leu Ala Asn Ala Ile Met Gly Ile Glu Ala Gly Ala
Thr Leu Val Asp Ala Thr Val Asn Gly Ile Gly Glu Arg Ala Gly Ile
Val Asp Leu Ser His Leu Leu Ala Ala Leu Tyr Tyr His Tyr Gly Val
Lys Lys Tyr Arg Leu Glu Lys Leu Tyr Ser Leu Ser Arg Leu Val Ser
Glu Ile Thr Gly Leu Gln Val Gln Val Asn Tyr Pro Ile Val Gly Gln
Asn Ala Phe Thr His Lys Ala Gly Leu His Val Ser Ala Val Val Arg
Asp Pro Ser Phe Tyr Glu Phe Leu Pro Ala Glu Thr Phe Gly Arg Glu
Arg Thr Ile Tyr Val Asp Arg Phe Ala Gly Arg Glu Thr Ile Arg Phe
His Leu Ser Arg Phe Gly Ile His Asp Glu Glu Ile Ile Glu Glu Leu
Leu Arg Arg Val Lys Ala Ser Arg Arg Pro Phe Thr Pro Glu Met Leu
Ala Glu Glu Ala Arg Arg Met Met Thr
SEQ ID NO: 74
PRT - Pyrococcus horikoshii
Met Ile Leu Asp Ser Thr Leu Arg Glu Gly Glu Gln Thr Pro Gly Val
Asn Tyr Ser Pro Glu Gln Arg Leu Arg Ile Ala Leu Ala Leu Asp Glu
Ile Gly Val Asp Phe Ile Glu Val Gly His Pro Ala Val Ser Lys Asp
Val Phe Ile Gly Ile Lys Leu Ile Ala Ser Gln Asp Leu Asn Ala Asn
Leu Leu Ala His Ser Arg Ala Leu Leu Glu Asp Ile Asp Tyr Val Ile
Gln Ala Asp Val Glu Trp Val Gly Ile Phe Phe Cys Leu Ser Asn Ala
Cys Leu Arg Lys Arg Phe Arg Met Ser Leu Ser Gln Ala Leu Glu Arg
Ile Ser Lys Ala Ile Glu Tyr Ala Lys Asp His Gly Leu Lys Val Arg
Phe Thr Pro Glu Asp Thr Thr Arg Thr Glu Trp Glu Asn Leu Arg Arg
Ala Ile Glu Leu Ala Lys Glu Leu Lys Val Asp Arg Ile Ser Val Ala
Asp Thr Thr Gly Gly Thr His Pro Leu Arg Phe Tyr Thr Leu Val Lys
Lys Val Val Asn Phe Gly Ile Pro Val Asn Val His Cys His Asn Asp
Leu Gly Leu Ala Leu Ala Asn Ala Ile Met Gly Ile Glu Gly Gly Ala
Thr Val Val Asp Ala Thr Val Asn Gly Leu Gly Glu Arg Ala Gly Ile
Val Asp Leu Ala Gln Ile Val Thr Val Leu Tyr Tyr His Tyr Gly Val
Lys Lys Tyr Arg Leu Asp Lys Leu Tyr Glu Ile Ser Arg Met Val Ser
Glu Ile Thr Gly Ile Ala Leu Gln Pro Asn Tyr Pro Ile Val Gly Glu
Asn Ala Phe Thr His Lys Ala Gly Leu His Val Ser Ala Val Leu Lys
Asp Pro Arg Phe Tyr Glu Phe Leu Pro Ala Glu Val Phe Gly Arg Glu
Arg Thr Ile Tyr Val Asp Arg Phe Ala Gly Lys Asp Thr Ile Arg Tyr
Tyr Leu Gln Lys Leu Gly Ile Asn Asp Glu Glu Phe Val Lys Val Leu
Leu Lys Arg Val Lys Ser Ser Arg Glu Pro Phe Thr Trp Asp Lys Phe
Ile Glu Glu Val Arg Arg Leu Lys Thr
SEQ ID NO: 75
PRT - Azotobacter vinelandii
Met Ala Ser Val Ile Ile Asp Asp Thr Thr Leu Arg Asp Gly Glu Gln
Ser Ala Gly Val Ala Phe Asn Ala Asp Glu Lys Ile Ala Ile Ala Arg
Ala Leu Ala Glu Leu Gly Val Pro Glu Leu Glu Ile Gly Ile Pro Ser
Met Gly Glu Glu Glu Arg Glu Val Met His Ala Ile Ala Gly Leu Gly
Leu Ser Ser Arg Leu Leu Ala Trp Cys Arg Leu Cys Asp Val Asp Leu
Ala Ala Ala Arg Ser Thr Gly Val Thr Met Val Asp Leu Ser Leu Pro
Val Ser Asp Leu Met Leu His His Lys Leu Asn Arg Asp Arg Asp Trp
Ala Leu Arg Glu Val Ala Arg Leu Val Gly Glu Ala Arg Met Ala Gly
Leu Glu Val Cys Leu Gly Cys Glu Asp Ala Ser Arg Ala Asp Leu Glu
Phe Val Val Gln Val Gly Glu Val Ala Gln Ala Ala Gly Ala Arg Arg
Leu Arg Phe Ala Asp Thr Val Gly Val Met Glu Pro Phe Gly Met Leu
Asp Arg Phe Arg Phe Leu Ser Arg Arg Leu Asp Met Glu Leu Glu Val
His Ala His Asp Asp Phe Gly Leu Ala Thr Ala Asn Thr Leu Ala Ala
Val Met Gly Gly Ala Thr His Ile Asn Thr Thr Val Asn Gly Leu Gly
Glu Arg Ala Gly Asn Ala Ala Leu Glu Glu Cys Val Leu Ala Leu Lys
Asn Leu His Gly Ile Asp Thr Gly Ile Asp Thr Arg Gly Ile Pro Ala
Ile Ser Ala Leu Val Glu Arg Ala Ser Gly Arg Gln Val Ala Trp Gln
Lys Ser Val Val Gly Ala Gly Val Phe Thr His Glu Ala Gly Ile His
Val Asp Gly Leu Leu Lys His Arg Arg Asn Tyr Glu Gly Leu Asn Pro
Asp Glu Leu Gly Arg Ser His Ser Leu Val Leu Gly Lys His Ser Gly
Ala His Met Val Arg Asn Thr Tyr Arg Asp Leu Gly Ile Glu Leu Ala
Asp Trp Gln Ser Gln Ala Leu Leu Gly Arg Ile Arg Ala Phe Ser Thr
Arg Thr Lys Arg Arg Ser Pro Gln Pro Ala Glu Leu Gln Asp Phe Tyr
Arg Gln Leu Cys Glu Gln Gly Asn Pro Glu Leu Ala Ala Gly Gly Met
Ala
SEQ ID NO: 76
PRT - Klebsiella pneumoniae
Met Glu Arg Val Leu Ile Asn Asp Thr Thr Leu Arg Asp Gly Glu Gln
Ser Pro Gly Val Ala Phe Arg Thr Ser Glu Lys Val Ala Ile Ala Glu
Ala Leu Tyr Ala Ala Gly Ile Thr Ala Met Glu Val Gly Thr Pro Ala
Met Gly Asp Glu Glu Ile Ala Arg Ile Gln Leu Val Arg Arg Gln Leu
Pro Asp Ala Thr Leu Met Thr Trp Cys Arg Met Asn Ala Leu Glu Ile
Arg Gln Ser Ala Asp Leu Gly Ile Asp Trp Val Asp Ile Ser Ile Pro
Ala Ser Asp Lys Leu Arg Gln Tyr Lys Leu Arg Glu Pro Leu Ala Val
Leu Leu Glu Arg Leu Ala Met Phe Ile His Leu Ala His Thr Leu Gly
Leu Lys Val Cys Ile Gly Cys Glu Asp Ala Ser Arg Ala Ser Gly Gln
Thr Leu Arg Ala Ile Ala Glu Val Ala Gln Asn Ala Pro Ala Ala Arg
Leu Arg Tyr Ala Asp Thr Val Gly Leu Leu Asp Pro Phe Thr Thr Ala
Ala Gln Ile Ser Ala Leu Arg Asp Val Trp Ser Gly Glu Ile Glu Met
His Ala His Asn Asp Leu Gly Met Ala Thr Ala Asn Thr Leu Ala Ala
Val Ser Ala Gly Ala Thr Ser Val Asn Thr Thr Val Leu Gly Leu Gly
Glu Arg Ala Gly Asn Ala Ala Ala Trp Lys Pro Ser Ala Leu Gly Leu
Glu Arg Cys Leu Gly Val Glu Thr Gly Val His Phe Ser Ala Leu Pro
Ala Leu Cys Gln Arg Val Ala Glu Ala Ala Gln Arg Ala Ile Asp Pro
Gln Gln Pro Leu Val Gly Glu Leu Val Phe Thr His Glu Ser Gly Val
His Val Ala Ala Leu Leu Arg Asp Ser Glu Ser Tyr Gln Ser Ile Ala
Pro Ser Leu Met Gly Arg Ser Tyr Arg Leu Val Leu Gly Lys His Ser
Gly Arg Gln Ala Val Asn Gly Val Phe Asp Gln Met Gly Tyr His Leu
Asn Ala Ala Gln Ile Asn Gln Leu Leu Pro Ala Ile Arg Arg Phe Ala
Glu Asn Trp Lys Arg Ser Pro Lys Asp Tyr Glu Leu Val Ala Ile Tyr
Asp Glu Leu Cys Gly Glu Ser Ala Leu Arg Ala Arg Gly
SEQ ID NO: 77
PRT - Pseudomonas stutzerii
Met Ser Ile Val Ile Asp Asp Thr Thr Leu Arg Asp Gly Glu Gln Ser
Ala Gly Val Ala Phe Ser Ala Glu Glu Lys Leu Ala Ile Ala Arg Ala
Leu Ala Gln Leu Gly Val Pro Glu Leu Glu Ile Gly Ile Pro Ser Met
Gly Glu Glu Glu Cys Glu Val Met Arg Ala Ile Ala Gly Leu Ala Leu
Pro Val Arg Leu Leu Ala Trp Cys Arg Leu Cys Asp Ala Asp Leu Leu
Ala Ala Gly Gly Thr Gly Val Gly Met Val Asp Leu Ser Leu Pro Val
Ser Asp Leu Met Leu Gln His Lys Leu Gly Arg Asp Arg Asp Trp Ala
Leu Arg Glu Ala Ala Arg Leu Val Gly Ala Ala Arg Asp Ala Gly Leu
Glu Val Cys Leu Gly Cys Glu Asp Ala Ser Arg Ala Asp Pro Glu Phe
Ile Val Arg Val Ala Glu Val Ala Gln Ala Ala Gly Ala Arg Arg Leu
Arg Phe Ala Asp Thr Val Gly Val Met Glu Pro Phe Ala Met His Ala
Arg Phe Arg Phe Leu Ala Glu Arg Leu Asp Leu Glu Leu Glu Val His
Ala His Asp Asp Phe Gly Leu Ala Thr Ala Asn Thr Leu Ala Ala Val
Arg Gly Gly Ala Thr His Ile Asn Thr Thr Val Asn Gly Leu Gly Glu
Arg Ala Gly Asn Ala Ala Leu Glu Glu Cys Ala Leu Ala Leu Lys His
Leu His Gly Ile Asp Cys Gly Ile Asp Val Arg Gly Ile Pro Ser Ile
Ser Ala Leu Val Glu Gln Ala Ser Gly Arg Gln Val Ala Trp Gln Lys
Ser Val Val Gly Ala Gly Val Phe Thr His Glu Ala Gly Ile His Val
Asp Gly Leu Leu Lys His Arg Arg Asn Tyr Glu Gly Leu Asn Pro Asp
Glu Leu Gly Arg Ser His Ser Leu Val Leu Gly Lys His Ser Gly Ala
His Met Val Glu Leu Ser Tyr Arg Glu Leu Gly Ile Glu Leu Gln Gln
Trp Gln Ser Arg Ala Leu Leu Gly Cys Ile Arg Arg Phe Ser Thr Gln
Thr Lys Arg Ser Pro Gln Ser Ala Asp Leu Gln Gly Phe Tyr Gln Gln
Leu Cys Glu Gln Gly Leu Ala Leu Ala Gly Gly Ala Ala
SEQ ID NO: 78
PRT - Acinetobacter sp. NCIMB9871
Met Asn Tyr Pro Asn Ile Pro Leu Tyr Ile Asn Gly Glu Phe Leu Asp
His Thr Asn Arg Asp Val Lys Glu Val Phe Asn Pro Val Asn His Glu
Cys Ile Gly Leu Met Ala Cys Ala Ser Gln Ala Asp Leu Asp Tyr Ala
Leu Glu Ser Ser Gln Gln Ala Phe Leu Arg Trp Lys Lys Thr Ser Pro
Ile Thr Arg Ser Glu Ile Leu Arg Thr Phe Ala Lys Leu Ala Arg Glu
Lys Ala Ala Glu Ile Gly Arg Asn Ile Thr Leu Asp Gln Gly Lys Pro
Leu Lys Glu Ala Ile Ala Glu Val Thr Val Cys Ala Glu His Ala Glu
Trp His Ala Glu Glu Cys Arg Arg Ile Tyr Gly Arg Val Ile Pro Pro
Arg Asn Pro Asn Val Gln Gln Leu Val Val Arg Glu Pro Leu Gly Val
Cys Leu Ala Phe Ser Pro Trp Asn Phe Pro Phe Asn Gln Ala Ile Arg
Lys Ile Ser Ala Ala Ile Ala Ala Gly Cys Thr Ile Ile Val Lys Gly
Ser Gly Asp Thr Pro Ser Ala Val Tyr Ala Ile Ala Gln Leu Phe His
Glu Ala Gly Leu Pro Asn Gly Val Leu Asn Val Ile Trp Gly Asp Ser
Asn Phe Ile Ser Asp Tyr Met Ile Lys Ser Pro Ile Ile Gln Lys Ile
Ser Phe Thr Gly Ser Thr Pro Val Gly Lys Lys Leu Ala Ser Gln Ala
Ser Leu Tyr Met Lys Pro Cys Thr Met Glu Leu Gly Gly His Ala Pro
Val Ile Val Cys Asp Asp Ala Asp Ile Asp Ala Ala Val Glu His Leu
Val Gly Tyr Lys Phe Arg Asn Ala Gly Gln Val Cys Val Ser Pro Thr
Arg Phe Tyr Val Gln Glu Gly Ile Tyr Lys Glu Phe Ser Glu Lys Val
Val Leu Arg Ala Lys Gln Ile Lys Val Gly Cys Gly Leu Asp Ala Ser
Ser Asp Met Gly Pro Leu Ala Gln Ala Arg Arg Met His Ala Met Gln
Gln Ile Val Glu Asp Ala Val His Lys Gly Ser Lys Leu Leu Leu Gly
Gly Asn Lys Ile Ser Asp Lys Gly Asn Phe Phe Glu Pro Thr Val Leu
Gly Asp Leu Cys Asn Asp Thr Gln Phe Met Asn Asp Glu Pro Phe Gly
Pro Ile Ile Gly Leu Ile Pro Phe Asp Thr Ile Asp His Val Leu Glu
Glu Ala Asn Arg Leu Pro Phe Gly Leu Ala Ser Tyr Ala Phe Thr Thr
Ser Ser Lys Asn Ala His Gln Ile Ser Tyr Gly Leu Glu Ala Gly Met
Val Ser Ile Asn His Met Gly Leu Ala Leu Ala Glu Thr Pro Phe Gly
Gly Ile Lys Asp Ser Gly Phe Gly Ser Glu Gly Gly Ile Glu Thr Phe
Asp Gly Tyr Leu Arg Thr Lys Phe Ile Thr Gln Leu Asn
SEQ ID NO: 79
PRT - Brucella melitensis 16M
Met Arg Ile Gly Lys Met Glu Met Gln Thr Arg Tyr Pro Asp Val Lys
Leu Phe Ile Asp Gly Thr Trp Arg Asp Gly Ser Arg Gly Glu Thr Ile
Glu Ile Phe Asn Pro Ala Thr Asp Glu Val Ile Gly His Ile Ala Arg
Ala Thr Thr Ala Asp Leu Asp Asp Ala Leu Ala Ala Val Asp Arg Gly
Phe Glu Ala Trp Ser Lys Val Ser Ala Phe Asp Arg Tyr Lys Ile Met
Arg Arg Ala Ala Asp Ile Phe Arg Ser Arg Gly Glu Glu Val Ala Arg
Leu Leu Thr Met Glu Gln Gly Lys Pro Leu Ala Glu Ala Arg Ile Glu
Ala Ala Ala Ala Cys Asp Leu Ile Asp Trp Phe Ala Glu Glu Ala Arg
Arg Ser Tyr Gly Arg Ile Val Pro Pro Arg Gln Ala Tyr Val Met Gln
Ala Glu Val Lys Glu Pro Val Gly Pro Val Ala Ala Phe Thr Pro Trp
Asn Phe Pro Ile Asn Gln Ala Val Arg Lys Ile Ser Ala Ala Leu Ala
Ala Gly Cys Ser Ile Leu Leu Lys Ala Ala Glu Asp Thr Pro Ala Ala
Pro Ala Glu Leu Val Arg Ala Phe Ala Glu Ala Gly Leu Pro Asp Gly
Ala Ile Asn Leu Val Tyr Gly Asp Pro Ala Glu Ile Ser Ala Tyr Leu
Ile Pro His Pro Val Ile Arg Lys Val Ser Phe Thr Gly Ser Thr Gln
Val Gly Lys Gln Leu Ala Ala Leu Ala Gly Leu His Met Lys Arg Val
Thr Met Glu Leu Gly Gly His Ala Pro Val Ile Ile Ala Ala Asp Ala
Asp Val Glu Gln Ala Ile Lys Val Val Ser Gly Ser Lys Phe Arg Asn
Ala Gly Gln Val Cys Ile Ser Pro Thr Arg Phe Leu Ile Glu Asn Ser
Val Tyr Asp Gln Val Val Glu Gly Met Ala Ala Tyr Ala Thr Ser Leu
Lys Val Gly Asp Gly Leu Glu Ala Gly Thr Thr Met Gly Pro Leu Val
Asn Ala Lys Arg Val Asn Ala Met Glu Arg Leu Val Gln Asp Ala Arg
Glu His Lys Ala Arg Val Val Thr Gly Gly Glu Arg Ile Gly Asn Arg
Gly Asn Phe Phe Glu Pro Thr Ile Leu Ala Asp Val Pro Arg Asp Ala
Ala Ile Met Asn Glu Glu Pro Phe Gly Pro Val Ala Leu Leu Asn Arg
Phe Asp Ala Leu Asp Glu Ala Leu Ser Glu Ala Asn Arg Leu Asn Tyr
Gly Leu Ala Ala Tyr Ala Phe Thr Gly Ser Ser Ala Lys Ala Ala Arg
Ile Ser Ser Thr Val Arg Ser Gly Met Ile Thr Ile Asn Gln Leu Arg
Ser Gly Pro Ala Gly Ser Ala Leu Arg Arg Asp Gln Arg Phe Arg Leu
Trp Asn Gly Arg Arg Cys Arg Arg Ala
SEQ ID NO: 80
PRT - Acinetobacter baumannii
Met Arg Leu Ile Met Leu Asn Ile Thr Gly Gln Asn Phe Ile Ala Gly
Gln Arg Ser Ser Ala Gly Ser Lys Phe Val Leu Ser Tyr Asp Ala Ala
Thr Asp Glu Ala Leu Pro Tyr Gln Phe Ala Gln Ala Thr Pro Glu Glu
Ile Asp Gln Ala Ala Gln Ala Ala Ala Leu Ala Tyr Pro Ala Phe Arg
Gln Thr Thr Pro Glu Gln Arg Ala Val Phe Leu Glu Thr Ile Ala Ser
Glu Ile Asp Ala Leu Asp Asp Gln Phe Ile Ala Thr Val Cys Gln Glu
Thr Ala Leu Pro Glu Ala Arg Ile Arg Gly Glu Arg Gly Arg Thr Thr
Gly Gln Leu Arg Leu Phe Ala Gln Val Leu Arg Arg Gly Asp Tyr Leu
Gly Ala Arg Ile Asp Leu Ala Leu Pro Glu Arg Gln Pro Leu Pro Arg
Pro Asp Leu Arg Gln Tyr Lys Ile Gly Val Gly Pro Val Ala Val Phe
Gly Ala Ser Asn Phe Pro Leu Ala Phe Ser Thr Ala Gly Gly Asp Thr
Ala Ser Ala Leu Ala Ala Gly Cys Pro Val Ile Val Lys Ala His Ser
Gly His Met Ala Thr Ala Glu Ser Ile Ala Asn Ala Ile Cys Ser Ala
Ile Glu Lys Cys Ala Met Pro Lys Gly Ile Phe Ser Met Ile Tyr Gly
Gln Gly Val Gly Glu Pro Leu Val Lys His Pro Ala Ile Lys Ala Val
Gly Phe Thr Gly Ser Leu Lys Gly Gly Arg Ala Leu Cys Asp Leu Ala
Ala Ala Arg Pro Glu Pro Ile Pro Val Phe Ala Glu Met Ser Ser Ile
Asn Pro Met Ile Leu Leu Pro Glu Ala Leu Lys Val Arg Gly Asp Lys
Ile Ala Thr Glu Leu Ser Gly Ser Val Val Leu Gly Cys Gly Gln Phe
Cys Thr Asn Pro Gly Leu Ile Ile Gly Ile Lys Ser Pro Glu Phe Ser
Gln Phe Leu Asp His Phe Lys Ala Ala Met Ala Gln Gln Pro Pro Gln
Thr Met Leu Asn Lys Gly Thr Leu Arg Ser Tyr Glu His Gly Leu Lys
Glu Leu Leu Ala His Asp Lys Ile Glu His Leu Ala Gly Gln Pro Gln
Gln Gly Pro Gln Ala Tyr Pro Gln Leu Phe Lys Ala Asp Val Ser Leu
Leu Leu Glu His Asp Glu Phe Leu Gln Glu Glu Val Phe Gly Pro Thr
Thr Ile Val Ile Glu Val Glu Ser Ala Glu Gln Leu Ala Leu Ala Leu
Asn Gly Leu Arg Gly Gln Leu Thr Ala Ser Leu Ile Ala Glu Pro Gln
Asp Phe Glu Asn Phe Ala Thr Leu Ile Pro Leu Leu Glu Glu Lys Ala
Gly Arg Leu Leu Leu Asn Gly Tyr Pro Thr Gly Val Glu Val Cys Asp
Ala Met Val His Gly Gly Pro Tyr Pro Ala Thr Ser Asp Ala Arg Gly
Thr Ser Val Gly Thr Leu Ala Ile Glu Arg Tyr Leu Arg Pro Val Cys
Tyr Gln Asn Tyr Pro Asp His Leu Leu Pro Leu Ala Leu Gln Asn Ala
Asn Pro Leu Gly Ile Ala Arg Leu Val Asn Gly Glu Met Ser Lys Ala
Ala Leu
SEQ ID NO: 81
PRT - Azospirillum brasilense
Met Ala Asn Val Thr Tyr Thr Asp Thr Gln Leu Leu Ile Asp Gly Glu
Trp Val Asp Ala Ala Ser Gly Lys Thr Ile Asp Val Val Asn Pro Ala
Thr Gly Lys Pro Ile Gly Arg Val Ala His Ala Gly Ile Ala Asp Leu
Asp Arg Ala Leu Ala Ala Ala Gln Ser Gly Phe Glu Ala Trp Arg Lys
Val Pro Ala His Glu Arg Ala Ala Thr Met Arg Lys Ala Ala Ala Leu
Val Arg Glu Arg Ala Asp Ala Ile Ala Gln Leu Met Thr Gln Glu Gln
Gly Lys Pro Leu Thr Glu Ala Arg Val Glu Val Leu Ser Ala Ala Asp
Ile Ile Glu Trp Phe Ala Asp Glu Gly Arg Arg Val Tyr Gly Arg Ile
Val Pro Pro Arg Asn Leu Gly Ala Gln Gln Thr Val Val Lys Glu Pro
Val Gly Pro Val Ala Ala Phe Thr Pro Trp Asn Phe Pro Val Asn Gln
Val Val Arg Lys Leu Ser Ala Ala Leu Ala Thr Gly Cys Ser Phe Leu
Val Lys Ala Pro Glu Glu Thr Pro Ala Ser Pro Ala Ala Leu Leu Arg
Ala Phe Val Asp Ala Gly Val Pro Ala Gly Val Ile Gly Leu Val Tyr
Gly Asp Pro Ala Glu Ile Ser Ser Tyr Leu Ile Pro His Pro Val Ile
Arg Lys Val Thr Phe Thr Gly Ser Thr Pro Val Gly Lys Gln Leu Ala
Ser Leu Ala Gly Leu His Met Lys Arg Ala Thr Met Glu Leu Gly Gly
His Ala Pro Val Ile Val Ala Glu Asp Ala Asp Val Ala Leu Ala Val
Lys Ala Ala Gly Gly Ala Lys Phe Arg Asn Ala Gly Gln Val Cys Ile
Ser Pro Thr Arg Phe Leu Val His Asn Ser Ile Arg Asp Glu Phe Thr
Arg Ala Leu Val Lys His Ala Glu Gly Leu Lys Val Gly Asn Gly Leu
Glu Glu Gly Thr Thr Leu Gly Ala Leu Ala Asn Pro Arg Arg Leu Thr
Ala Met Ala Ser Val Ile Asp Asn Ala Arg Lys Val Gly Ala Ser Ile
Glu Thr Gly Gly Glu Arg Ile Gly Ser Glu Gly Asn Phe Phe Ala Pro
Thr Val Ile Ala Asn Val Pro Leu Asp Ala Asp Val Phe Asn Asn Glu
Pro Phe Gly Pro Val Ala Ala Ile Arg Gly Phe Asp Lys Leu Glu Glu
Ala Ile Ala Glu Ala Asn Arg Leu Pro Phe Gly Leu Ala Gly Tyr Ala
Phe Thr Arg Ser Phe Ala Asn Val His Leu Leu Thr Gln Arg Leu Glu
Val Gly Met Leu Trp Ile Asn Gln Pro Ala Thr Pro Trp Pro Glu Met
Pro Phe Gly Gly Val Lys Asp Ser Gly Tyr Gly Ser Glu Gly Gly Pro
Glu Ala Leu Glu Pro Tyr Leu Val Thr Lys Ser Val Thr Val Met Ala
Val
SEQ ID NO: 82
DNA - Bacillus weihenstephanensis
gtg caa gcg acg gag caa aca caa agt ttg aaa aaa aca gat gaa aag
Val Gln Ala Thr Glu Gln Thr Gln Ser Leu Lys Lys Thr Asp Glu Lys
tac ctt tgg cat gcg atg aga gga gca gcc cct agt cca acg aat tta
Tyr Leu Trp His Ala Met Arg Gly Ala Ala Pro Ser Pro Thr Asn Leu
att atc aca aaa gca gaa ggg gca tgg gtg acg gat att gat gga aac
Ile Ile Thr Lys Ala Glu Gly Ala Trp Val Thr Asp Ile Asp Gly Asn
cgt tat tta gac ggt atg tcc ggt ctt tgg tgc gtg aat gtt ggg tat
Arg Tyr Leu Asp Gly Met Ser Gly Leu Trp Cys Val Asn Val Gly Tyr
ggt cga aaa gaa ctt gca aga gcg gcg ttt gaa cag ctt gaa gaa atg
Gly Arg Lys Glu Leu Ala Arg Ala Ala Phe Glu Gln Leu Glu Glu Met
ccg tat ttc cct ctg act caa agt cat gtt cct gct att aaa tta gca
Pro Tyr Phe Pro Leu Thr Gln Ser His Val Pro Ala Ile Lys Leu Ala
gaa aaa ttg aat gaa tgg ctt gat gat gaa tac gtc att ttc ttt tct
Glu Lys Leu Asn Glu Trp Leu Asp Asp Glu Tyr Val Ile Phe Phe Ser
aac agt gga tcg gaa gcg aat gaa aca gca ttt aaa att gct cgt caa
Asn Ser Gly Ser Glu Ala Asn Glu Thr Ala Phe Lys Ile Ala Arg Gln
tat cat caa caa aaa ggt gat cat gga cgc tat aag ttt att tcc cgc
Tyr His Gln Gln Lys Gly Asp His Gly Arg Tyr Lys Phe Ile Ser Arg
tac cgc gct tat cac ggt aac tca atg gga gct ctt gca gca aca ggt
Tyr Arg Ala Tyr His Gly Asn Ser Met Gly Ala Leu Ala Ala Thr Gly
caa gca cag cga aag tat aaa tat gaa cca ctc ggg caa gga ttc ctg
Gln Ala Gln Arg Lys Tyr Lys Tyr Glu Pro Leu Gly Gln Gly Phe Leu
cat gta gca ccg cct gat acg tat cga aat cca gag gat gtt cat aca
His Val Ala Pro Pro Asp Thr Tyr Arg Asn Pro Glu Asp Val His Thr
ctg gca agt gct gag gaa atc gat cgt gtc atg aca tgg gag tta agc
Leu Ala Ser Ala Glu Glu Ile Asp Arg Val Met Thr Trp Glu Leu Ser
caa aca gta gcc ggt gtg att atg gag cca atc att act ggg ggc gga
Gln Thr Val Ala Gly Val Ile Met Glu Pro Ile Ile Thr Gly Gly Gly
att tta atg cct cct gat gga tat atg gga aaa gta aaa gaa att tgc
Ile Leu Met Pro Pro Asp Gly Tyr Met Gly Lys Val Lys Glu Ile Cys
gag aag cac ggt gcg ttg ctc att tgt gat gaa gtt ata tgt gga ttt
Glu Lys His Gly Ala Leu Leu Ile Cys Asp Glu Val Ile Cys Gly Phe
ggc cgg aca ggg aag cca ttt gga ttt atg aat tat ggc gtc aaa cca
Gly Arg Thr Gly Lys Pro Phe Gly Phe Met Asn Tyr Gly Val Lys Pro
gat atc att aca atg gca aaa ggt att aca agt gcg tat ctt cct ttg
Asp Ile Ile Thr Met Ala Lys Gly Ile Thr Ser Ala Tyr Leu Pro Leu
tca gca aca gca gtt aga cga gag gtt tat gag gca ttc gta ggt agt
Ser Ala Thr Ala Val Arg Arg Glu Val Tyr Glu Ala Phe Val Gly Ser
gat gat tat gat cgc ttc cgc cat gta aat acg ttc gga ggg aat cct
Asp Asp Tyr Asp Arg Phe Arg His Val Asn Thr Phe Gly Gly Asn Pro
gct gct tgc gct tta gct ttg aag aat tta gaa att atg gag aat gag
Ala Ala Cys Ala Leu Ala Leu Lys Asn Leu Glu Ile Met Glu Asn Glu
aaa ctc att gaa cgt tcc aaa gaa ttg ggt gaa cga ctg tta tat gag
Lys Leu Ile Glu Arg Ser Lys Glu Leu Gly Glu Arg Leu Leu Tyr Glu
cta gag gat gta aaa gag cat cca aac gta ggg gat gtt cgc gga aag
Leu Glu Asp Val Lys Glu His Pro Asn Val Gly Asp Val Arg Gly Lys
ggc ctt ctt tta ggc att gaa cta gtg gaa gat aag caa aca aaa gaa
Gly Leu Leu Leu Gly Ile Glu Leu Val Glu Asp Lys Gln Thr Lys Glu
ccg gct tcc att gaa aag atg aac aaa gtc atc aat gct tgt aaa gaa
Pro Ala Ser Ile Glu Lys Met Asn Lys Val Ile Asn Ala Cys Lys Glu
aaa ggt cta att att ggt aaa aat ggt gac act gtc gca ggt tac aat
Lys Gly Leu Ile Ile Gly Lys Asn Gly Asp Thr Val Ala Gly Tyr Asn
aat att ttg cag ctt gca cct cca tta agc atc aca gag gaa gac ttt
Asn Ile Leu Gln Leu Ala Pro Pro Leu Ser Ile Thr Glu Glu Asp Phe
act ttt atc gtt aaa aca atg aaa gaa tgt tta tcc cgc att aac ggg
Thr Phe Ile Val Lys Thr Met Lys Glu Cys Leu Ser Arg Ile Asn Gly
cag taa
Gln
SEQ ID NO: 83
PRT - Bacillus weihenstephanensis
Val Gln Ala Thr Glu Gln Thr Gln Ser Leu Lys Lys Thr Asp Glu Lys
Tyr Leu Trp His Ala Met Arg Gly Ala Ala Pro Ser Pro Thr Asn Leu
Ile Ile Thr Lys Ala Glu Gly Ala Trp Val Thr Asp Ile Asp Gly Asn
Arg Tyr Leu Asp Gly Met Ser Gly Leu Trp Cys Val Asn Val Gly Tyr
Gly Arg Lys Glu Leu Ala Arg Ala Ala Phe Glu Gln Leu Glu Glu Met
Pro Tyr Phe Pro Leu Thr Gln Ser His Val Pro Ala Ile Lys Leu Ala
Glu Lys Leu Asn Glu Trp Leu Asp Asp Glu Tyr Val Ile Phe Phe Ser
Asn Ser Gly Ser Glu Ala Asn Glu Thr Ala Phe Lys Ile Ala Arg Gln
Tyr His Gln Gln Lys Gly Asp His Gly Arg Tyr Lys Phe Ile Ser Arg
Tyr Arg Ala Tyr His Gly Asn Ser Met Gly Ala Leu Ala Ala Thr Gly
Gln Ala Gln Arg Lys Tyr Lys Tyr Glu Pro Leu Gly Gln Gly Phe Leu
His Val Ala Pro Pro Asp Thr Tyr Arg Asn Pro Glu Asp Val His Thr
Leu Ala Ser Ala Glu Glu Ile Asp Arg Val Met Thr Trp Glu Leu Ser
Gln Thr Val Ala Gly Val Ile Met Glu Pro Ile Ile Thr Gly Gly Gly
Ile Leu Met Pro Pro Asp Gly Tyr Met Gly Lys Val Lys Glu Ile Cys
Glu Lys His Gly Ala Leu Leu Ile Cys Asp Glu Val Ile Cys Gly Phe
Gly Arg Thr Gly Lys Pro Phe Gly Phe Met Asn Tyr Gly Val Lys Pro
Asp Ile Ile Thr Met Ala Lys Gly Ile Thr Ser Ala Tyr Leu Pro Leu
Ser Ala Thr Ala Val Arg Arg Glu Val Tyr Glu Ala Phe Val Gly Ser
Asp Asp Tyr Asp Arg Phe Arg His Val Asn Thr Phe Gly Gly Asn Pro
Ala Ala Cys Ala Leu Ala Leu Lys Asn Leu Glu Ile Met Glu Asn Glu
Lys Leu Ile Glu Arg Ser Lys Glu Leu Gly Glu Arg Leu Leu Tyr Glu
Leu Glu Asp Val Lys Glu His Pro Asn Val Gly Asp Val Arg Gly Lys
Gly Leu Leu Leu Gly Ile Glu Leu Val Glu Asp Lys Gln Thr Lys Glu
Pro Ala Ser Ile Glu Lys Met Asn Lys Val Ile Asn Ala Cys Lys Glu
Lys Gly Leu Ile Ile Gly Lys Asn Gly Asp Thr Val Ala Gly Tyr Asn
Asn Ile Leu Gln Leu Ala Pro Pro Leu Ser Ile Thr Glu Glu Asp Phe
Thr Phe Ile Val Lys Thr Met Lys Glu Cys Leu Ser Arg Ile Asn Gly
Gln
SEQ ID NO: 84
DNA - Artificial
B. weihenstephanensis KBAB4 aminotransferase codon-optimised gene
atgcaggcta ccgaacaaac ccaatctctg aaaaagactg acgaaaaata tctgtggcac
gcgatgcgcg gtgcagctcc gtctccgacc aacctgatta ttaccaaagc tgaaggcgcg
tgggtgaccg acattgacgg taaccgttat ctggatggca tgagcggcct gtggtgtgtt
aatgtcggtt atggccgtaa ggagctggcg cgcgcggcat ttgaacaact ggaagaaatg
ccgtacttcc cgctgactca aagccatgtg ccggctatca aactggcgga aaaactgaac
gaatggctgg acgacgaata cgtgattttc ttctctaatt ctggctccga agcaaacgaa
accgcattca aaatcgcccg tcaatatcac cagcagaaag gtgaccacgg ccgctataaa
ttcatcagcc gttatcgtgc ataccatggt aattctatgg gtgcgctggc tgctaccggt
caggctcagc gcaaatacaa gtacgaaccg ctgggtcagg gttttctgca cgttgcacca
ccggatacct accgtaaccc ggaagacgtc cacaccctgg cttctgccga agaaatcgat
cgtgttatga cctgggagct gtcccagact gttgcgggtg ttatcatgga acctattatt
accggtggtg gcattctgat gccgccggac ggttatatgg gtaaagtcaa ggaaatctgc
gaaaaacacg gcgcgctgct gatctgcgat gaagttatct gtggcttcgg tcgcaccggc
aaaccatttg gcttcatgaa ttatggcgta aaacctgaca ttattaccat ggctaaaggc
attacttccg cttatctgcc gctgagcgcg accgcagttc gccgcgaagt ttatgaagcg
tttgttggtt ctgatgatta cgaccgtttc cgtcatgtaa acacgtttgg cggtaaccca
gcggcatgtg cgctggcgct gaaaaacctg gaaatcatgg aaaacgaaaa gctgatcgaa
cgtagcaaag aactgggtga acgtctgctg tacgaactgg aagatgtcaa agaacacccg
aacgtgggcg atgttcgcgg taaaggcctg ctgctgggta ttgaactggt tgaagacaaa
cagaccaagg aaccggcttc cattgaaaag atgaacaaag tgattaacgc gtgcaaagag
aaaggcctga tcattggtaa gaacggtgat accgtggcag gttataacaa cattctgcag
ctggcgccgc ctctgagcat cactgaagaa gatttcacct tcatcgtcaa aactatgaag
gagtgcctga gccgcatcaa tggtcagtaa
SEQ ID NO: 85
DNA - Pseudomonas aeruginosa
atg aac age caa ate acc aac gec aag acc cgt gag tgg cag gcg ttg
Met Asn Ser Gln Ile Thr Asn Ala Lys Thr Arg Glu Trp Gln Ala Leu
agc cgc gac cac cat ctg ccg ccg ttc acc gac tac aag cag ttg aac
Ser Arg Asp His His Leu Pro Pro Phe Thr Asp Tyr Lys Gln Leu Asn
gag aag ggc gcg cgg atc atc acc aag gcc gaa ggc gtc tat atc tgg
Glu Lys Gly Ala Arg Ile Ile Thr Lys Ala Glu Gly Val Tyr Ile Trp
gac agc gag ggc aac aag atc ctc gat gcg atg gcc ggc ctc tgg tgc
Asp Ser Glu Gly Asn Lys Ile Leu Asp Ala Met Ala Gly Leu Trp Cys
gtc aac gtc ggc tac ggc cgc gag gag ctg gtc cag gcc gcc acc cgg
Val Asn Val Gly Tyr Gly Arg Glu Glu Leu Val Gln Ala Ala Thr Arg
cag atg cgc gag ttg ccg ttc tac aac ctg ttc ttc cag acc gcc cac
Gln Met Arg Glu Leu Pro Phe Tyr Asn Leu Phe Phe Gln Thr Ala His
ccg ccg gtg gtc gag ctg gcc aag gcg atc gcc gac gtc gct ccg gaa
Pro Pro Val Val Glu Leu Ala Lys Ala Ile Ala Asp Val Ala Pro Glu
ggc atg aac cac gtg ttc ttc acc ggc tcc ggc tcc gag gcc aac gac
Gly Met Asn His Val Phe Phe Thr Gly Ser Gly Ser Glu Ala Asn Asp
acc gtg ctg cgt atg gtc cgc cac tat tgg gcg acc aag ggc cag ccg
Thr Val Leu Arg Met Val Arg His Tyr Trp Ala Thr Lys Gly Gln Pro
cag aag aaa gtg gtg atc ggc cgc tgg aac ggc tac cac ggc tcc acc
Gln Lys Lys Val Val Ile Gly Arg Trp Asn Gly Tyr His Gly Ser Thr
gtc gcc ggc gtc agc ctg ggc ggc atg aag gcg ttg cat gag cag ggt
Val Ala Gly Val Ser Leu Gly Gly Met Lys Ala Leu His Glu Gln Gly
gat ttc ccc atc ccg ggc atc gtc cac atc gcc cag ccc tac tgg tac
Asp Phe Pro Ile Pro Gly Ile Val His Ile Ala Gln Pro Tyr Trp Tyr
ggc gag ggc ggc gac atg tcg ccg gac gag ttc ggc gtc tgg gcc gcc
Gly Glu Gly Gly Asp Met Ser Pro Asp Glu Phe Gly Val Trp Ala Ala
gag cag ttg gag aag aag att ctc gaa gtg ggc gag gaa aac gtc gcc
Glu Gln Leu Glu Lys Lys Ile Leu Glu Val Gly Glu Glu Asn Val Ala
gcc ttc atc gcc gag ccg atc cag ggc gcc ggc ggc gtg atc gtc ccg
Ala Phe Ile Ala Glu Pro Ile Gln Gly Ala Gly Gly Val Ile Val Pro
ccg gac acc tac tgg ccg aag atc cgc gag atc ctc gcc aag tac gac
Pro Asp Thr Tyr Trp Pro Lys Ile Arg Glu Ile Leu Ala Lys Tyr Asp
atc ctg ttc atc gcc gac gaa gtg atc tgc ggc ttc ggc cgt acc ggc
Ile Leu Phe Ile Ala Asp Glu Val Ile Cys Gly Phe Gly Arg Thr Gly
gag tgg ttc ggc agc cag tac tac ggc aac gcc ccg gac ctg atg ccg
Glu Trp Phe Gly Ser Gln Tyr Tyr Gly Asn Ala Pro Asp Leu Met Pro
atc gcc aag ggc ctc acc tcc ggc tac atc ccc atg ggc ggg gtg gtg
Ile Ala Lys Gly Leu Thr Ser Gly Tyr Ile Pro Met Gly Gly Val Val
gtg cgc gac gag atc gtc gaa gtg ctc aac cag ggc ggc gag ttc tac
Val Arg Asp Glu Ile Val Glu Val Leu Asn Gln Gly Gly Glu Phe Tyr
cac ggc ttc acc tat tcc ggt cac ccg gtg gcg gcc gcc gtg gcc ctg
His Gly Phe Thr Tyr Ser Gly His Pro Val Ala Ala Ala Val Ala Leu
gag aac atc cgc atc ctg cgc gaa gag aag atc atc gag aag gtg aag
Glu Asn Ile Arg Ile Leu Arg Glu Glu Lys Ile Ile Glu Lys Val Lys
gcg gaa acg gca ccg tat ttg cag aaa cgc tgg cag gag ctg gcc gac
Ala Glu Thr Ala Pro Tyr Leu Gln Lys Arg Trp Gln Glu Leu Ala Asp
cac ccg ttg gtg ggc gaa gcg cgc ggg gtc ggc atg gtc gcc gcc ctg
His Pro Leu Val Gly Glu Ala Arg Gly Val Gly Met Val Ala Ala Leu
gag ctg gtc aag aac aag aag acc cgc gag cgt ttc acc gac aag ggc
Glu Leu Val Lys Asn Lys Lys Thr Arg Glu Arg Phe Thr Asp Lys Gly
gtc ggg atg ctg tgc cgg gaa cat tgt ttc cgc aac ggt ttg atc atg
Val Gly Met Leu Cys Arg Glu His Cys Phe Arg Asn Gly Leu Ile Met
cgc gcg gtg ggc gac act atg att atc tcg ccg ccg ctg gtg atc gat
Arg Ala Val Gly Asp Thr Met Ile Ile Ser Pro Pro Leu Val Ile Asp
ccg tcg cag atc gat gag ttg atc acc ctg gcg cgc aag tgc ctc gat
Pro Ser Gln Ile Asp Glu Leu Ile Thr Leu Ala Arg Lys Cys Leu Asp
cag acc gcc gcc gcc gtc ctg gct tga
Gln Thr Ala Ala Ala Val Leu Ala
SEQ ID NO: 86
PRT - Pseudomonas aeruginosa
Met Asn Ser Gln Ile Thr Asn Ala Lys Thr Arg Glu Trp Gln Ala Leu
Ser Arg Asp His His Leu Pro Pro Phe Thr Asp Tyr Lys Gln Leu Asn
Glu Lys Gly Ala Arg Ile Ile Thr Lys Ala Glu Gly Val Tyr Ile Trp
Asp Ser Glu Gly Asn Lys Ile Leu Asp Ala Met Ala Gly Leu Trp Cys
Val Asn Val Gly Tyr Gly Arg Glu Glu Leu Val Gln Ala Ala Thr Arg
Gln Met Arg Glu Leu Pro Phe Tyr Asn Leu Phe Phe Gln Thr Ala His
Pro Pro Val Val Glu Leu Ala Lys Ala Ile Ala Asp Val Ala Pro Glu
Gly Met Asn His Val Phe Phe Thr Gly Ser Gly Ser Glu Ala Asn Asp
Thr Val Leu Arg Met Val Arg His Tyr Trp Ala Thr Lys Gly Gln Pro
Gln Lys Lys Val Val Ile Gly Arg Trp Asn Gly Tyr His Gly Ser Thr
Val Ala Gly Val Ser Leu Gly Gly Met Lys Ala Leu His Glu Gln Gly
Asp Phe Pro Ile Pro Gly Ile Val His Ile Ala Gln Pro Tyr Trp Tyr
Gly Glu Gly Gly Asp Met Ser Pro Asp Glu Phe Gly Val Trp Ala Ala
Glu Gln Leu Glu Lys Lys Ile Leu Glu Val Gly Glu Glu Asn Val Ala
Ala Phe Ile Ala Glu Pro Ile Gln Gly Ala Gly Gly Val Ile Val Pro
Pro Asp Thr Tyr Trp Pro Lys Ile Arg Glu Ile Leu Ala Lys Tyr Asp
Ile Leu Phe Ile Ala Asp Glu Val Ile Cys Gly Phe Gly Arg Thr Gly
Glu Trp Phe Gly Ser Gln Tyr Tyr Gly Asn Ala Pro Asp Leu Met Pro
Ile Ala Lys Gly Leu Thr Ser Gly Tyr Ile Pro Met Gly Gly Val Val
Val Arg Asp Glu Ile Val Glu Val Leu Asn Gln Gly Gly Glu Phe Tyr
His Gly Phe Thr Tyr Ser Gly His Pro Val Ala Ala Ala Val Ala Leu
Glu Asn Ile Arg Ile Leu Arg Glu Glu Lys Ile Ile Glu Lys Val Lys
Ala Glu Thr Ala Pro Tyr Leu Gln Lys Arg Trp Gln Glu Leu Ala Asp
His Pro Leu Val Gly Glu Ala Arg Gly Val Gly Met Val Ala Ala Leu
Glu Leu Val Lys Asn Lys Lys Thr Arg Glu Arg Phe Thr Asp Lys Gly
Val Gly Met Leu Cys Arg Glu His Cys Phe Arg Asn Gly Leu Ile Met
Arg Ala Val Gly Asp Thr Met Ile Ile Ser Pro Pro Leu Val Ile Asp
Pro Ser Gln Ile Asp Glu Leu Ile Thr Leu Ala Arg Lys Cys Leu Asp
Gln Thr Ala Ala Ala Val Leu Ala
SEQ ID NO: 87
DNA - Artificial
Primer
ggggacaagt ttgtacaaaa aagcaggcta ggaggaatta accatgaaca gccaaatcac
caacgccaag
SEQ ID NO: 88
DNA - Artificial
Primer
ggggaccact ttgtacaaga aagctgggtt caagccagga cggcggcgg
SEQ ID NO: 89
DNA - Bacillus subtilis
atg aag gtt tta gtc aat ggc cgg ctg att ggg cgc agt gaa gca tca
Met Lys Val Leu Val Asn Gly Arg Leu Ile Gly Arg Ser Glu Ala Ser
atc gat ttg gaa gat cgc ggt tat cag ttt ggt gac ggc atc tat gaa
Ile Asp Leu Glu Asp Arg Gly Tyr Gln Phe Gly Asp Gly Ile Tyr Glu
gtg atc agg gtg tac aaa gga gta ttg ttc ggc tta cgt gag cat gca
Val Ile Arg Val Tyr Lys Gly Val Leu Phe Gly Leu Arg Glu His Ala
gag cgt ttt ttc aga agt gct gct gaa atc gga att tca ctg cca ttc
Glu Arg Phe Phe Arg Ser Ala Ala Glu Ile Gly Ile Ser Leu Pro Phe
agt ata gaa gat ctc gag tgg gac ctg caa aag ctt gta cag gaa aat
Ser Ile Glu Asp Leu Glu Trp Asp Leu Gln Lys Leu Val Gln Glu Asn
gcg gtc agt gag gga gcg gta tac att cag aca aca aga ggt gtg gcc
Ala Val Ser Glu Gly Ala Val Tyr Ile Gln Thr Thr Arg Gly Val Ala
ccg cga aaa cac cag tat gaa gcc ggc ctc gag ccg cag act act gcc
Pro Arg Lys His Gln Tyr Glu Ala Gly Leu Glu Pro Gln Thr Thr Ala
tat acg ttt acg gtg aaa aaa ccg gag caa gag cag gca tac gga gtg
Tyr Thr Phe Thr Val Lys Lys Pro Glu Gln Glu Gln Ala Tyr Gly Val
gcg gcc att aca gat gag gat ctt cgc tgg tta aga tgt gat atc aaa
Ala Ala Ile Thr Asp Glu Asp Leu Arg Trp Leu Arg Cys Asp Ile Lys
agt ctg aat tta ctg tat aat gtc atg acg aag caa agg gcc tat gaa
Ser Leu Asn Leu Leu Tyr Asn Val Met Thr Lys Gln Arg Ala Tyr Glu
gcc gga gca ttt gaa gcc att tta ctt agg gac ggc gtt gtt acg gag
Ala Gly Ala Phe Glu Ala Ile Leu Leu Arg Asp Gly Val Val Thr Glu
ggt aca tcc tct aac gtt tat gcc gtt atc aac ggc aca gtg cga aca
Gly Thr Ser Ser Asn Val Tyr Ala Val Ile Asn Gly Thr Val Arg Thr
cat ccg gct aat cgg ctc att ctc aat gga att aca cgg atg aat att
His Pro Ala Asn Arg Leu Ile Leu Asn Gly Ile Thr Arg Met Asn Ile
tta gga ctg att gag aag aat ggg atc aaa ctg gat gag act cct gtc
Leu Gly Leu Ile Glu Lys Asn Gly Ile Lys Leu Asp Glu Thr Pro Val
agt gaa gaa gag ttg aaa cag gcg gaa gag atc ttt att tcg tca acg
Ser Glu Glu Glu Leu Lys Gln Ala Glu Glu Ile Phe Ile Ser Ser Thr
acg gca gaa att att ccg gtc gtg acg ctc gat gga caa tcg atc gga
Thr Ala Glu Ile Ile Pro Val Val Thr Leu Asp Gly Gln Ser Ile Gly
agc ggg aaa ccc gga ccg gtg acc aaa cag ctt cag gct gct ttt caa
Ser Gly Lys Pro Gly Pro Val Thr Lys Gln Leu Gln Ala Ala Phe Gln
gaa agc att caa cag gct gct agc att tca taa
Glu Ser Ile Gln Gln Ala Ala Ser Ile Ser
SEQ ID NO: 90
PRT - Bacillus subtilis
Met Lys Val Leu Val Asn Gly Arg Leu Ile Gly Arg Ser Glu Ala Ser
Ile Asp Leu Glu Asp Arg Gly Tyr Gln Phe Gly Asp Gly Ile Tyr Glu
Val Ile Arg Val Tyr Lys Gly Val Leu Phe Gly Leu Arg Glu His Ala
Glu Arg Phe Phe Arg Ser Ala Ala Glu Ile Gly Ile Ser Leu Pro Phe
Ser Ile Glu Asp Leu Glu Trp Asp Leu Gln Lys Leu Val Gln Glu Asn
Ala Val Ser Glu Gly Ala Val Tyr Ile Gln Thr Thr Arg Gly Val Ala
Pro Arg Lys His Gln Tyr Glu Ala Gly Leu Glu Pro Gln Thr Thr Ala
Tyr Thr Phe Thr Val Lys Lys Pro Glu Gln Glu Gln Ala Tyr Gly Val
Ala Ala Ile Thr Asp Glu Asp Leu Arg Trp Leu Arg Cys Asp Ile Lys
Ser Leu Asn Leu Leu Tyr Asn Val Met Thr Lys Gln Arg Ala Tyr Glu
Ala Gly Ala Phe Glu Ala Ile Leu Leu Arg Asp Gly Val Val Thr Glu
Gly Thr Ser Ser Asn Val Tyr Ala Val Ile Asn Gly Thr Val Arg Thr
His Pro Ala Asn Arg Leu Ile Leu Asn Gly Ile Thr Arg Met Asn Ile
Leu Gly Leu Ile Glu Lys Asn Gly Ile Lys Leu Asp Glu Thr Pro Val
Ser Glu Glu Glu Leu Lys Gln Ala Glu Glu Ile Phe Ile Ser Ser Thr
Thr Ala Glu Ile Ile Pro Val Val Thr Leu Asp Gly Gln Ser Ile Gly
Ser Gly Lys Pro Gly Pro Val Thr Lys Gln Leu Gln Ala Ala Phe Gln
Glu Ser Ile Gln Gln Ala Ala Ser Ile Ser
SEQ ID NO: 91
DNA - Bacillus subtilis
atg act cat gat ttg ata gaa aaa agt aaa aag cac ctc tgg ctg cca
Met Thr His Asp Leu Ile Glu Lys Ser Lys Lys His Leu Trp Leu Pro
ttt acc caa atg aaa gat tat gat gaa aac ccc tta atc atc gaa agc
Phe Thr Gln Met Lys Asp Tyr Asp Glu Asn Pro Leu Ile Ile Glu Ser
ggg act gga atc aaa gtc aaa gac ata aac ggc aag gaa tac tat gac
Gly Thr Gly Ile Lys Val Lys Asp Ile Asn Gly Lys Glu Tyr Tyr Asp
ggt ttt tca tcg gtt tgg ctt aat gtc cac gga cac cgc aaa aaa gaa
Gly Phe Ser Ser Val Trp Leu Asn Val His Gly His Arg Lys Lys Glu
cta gat gac gcc ata aaa aaa cag ctc gga aaa att gcg cac tcc acg
Leu Asp Asp Ala Ile Lys Lys Gln Leu Gly Lys Ile Ala His Ser Thr
tta ttg ggc atg acc aat gtt cca gca acc cag ctt gcc gaa aca tta
Leu Leu Gly Met Thr Asn Val Pro Ala Thr Gln Leu Ala Glu Thr Leu
atc gac atc agc cca aaa aag ctc acg cgg gtc ttt tat tca gac agc
Ile Asp Ile Ser Pro Lys Lys Leu Thr Arg Val Phe Tyr Ser Asp Ser
ggc gca gag gcg atg gaa ata gcc cta aaa atg gcg ttt cag tat tgg
Gly Ala Glu Ala Met Glu Ile Ala Leu Lys Met Ala Phe Gln Tyr Trp
aag aac atc ggg aag ccc gag aaa caa aaa ttc atc gca atg aaa aac
Lys Asn Ile Gly Lys Pro Glu Lys Gln Lys Phe Ile Ala Met Lys Asn
ggg tat cac ggt gat acg att ggc gcc gtc agt gtc ggt tca att gag
Gly Tyr His Gly Asp Thr Ile Gly Ala Val Ser Val Gly Ser Ile Glu
ctt ttt cac cac gta tac ggc ccg ttg atg ttc gag agt tac aag gcc
Leu Phe His His Val Tyr Gly Pro Leu Met Phe Glu Ser Tyr Lys Ala
ccg att cct tat gtg tat cgt tct gaa agc ggt gat cct gat gag tgc
Pro Ile Pro Tyr Val Tyr Arg Ser Glu Ser Gly Asp Pro Asp Glu Cys
cgt gat cag tgc ctc cga gag ctt gca cag ctg ctt gag gaa cat cat
Arg Asp Gln Cys Leu Arg Glu Leu Ala Gln Leu Leu Glu Glu His His
gag gaa att gcc gcg ctt tcc att gaa tca atg gta caa ggc gcg tcc
Glu Glu Ile Ala Ala Leu Ser Ile Glu Ser Met Val Gln Gly Ala Ser
ggt atg atc gtg atg ccg gaa gga tat ttg gca ggc gtg cgc gag cta
Gly Met Ile Val Met Pro Glu Gly Tyr Leu Ala Gly Val Arg Glu Leu
tgt aca aca tac gat gtc tta atg atc gtt gat gaa gtc gct aca ggc
Cys Thr Thr Tyr Asp Val Leu Met Ile Val Asp Glu Val Ala Thr Gly
ttt ggc cgt aca gga aaa atg ttt gcg tgc gag cac gag aat gtc cag
Phe Gly Arg Thr Gly Lys Met Phe Ala Cys Glu His Glu Asn Val Gln
cct gat ctg atg gct gcc ggt aaa ggc att aca gga ggc tat ttg cca
Pro Asp Leu Met Ala Ala Gly Lys Gly Ile Thr Gly Gly Tyr Leu Pro
att gcc gtt acg ttt gcc act gaa gac atc tat aag gca ttc tat gat
Ile Ala Val Thr Phe Ala Thr Glu Asp Ile Tyr Lys Ala Phe Tyr Asp
gat tat gaa aac cta aaa acc ttt ttc cat ggc cat tcc tat aca ggc
Asp Tyr Glu Asn Leu Lys Thr Phe Phe His Gly His Ser Tyr Thr Gly
aat cag ctt ggc tgt gcg gtt gcg ctt gaa aat ctg gca tta ttt gaa
Asn Gln Leu Gly Cys Ala Val Ala Leu Glu Asn Leu Ala Leu Phe Glu
tct gaa aac att gtg gaa caa gta gcg gaa aaa agt aaa aag ctc cat
Ser Glu Asn Ile Val Glu Gln Val Ala Glu Lys Ser Lys Lys Leu His
ttt ctt ctt caa gat ctg cac gct ctt cct cat gtt ggg gat att cgg
Phe Leu Leu Gln Asp Leu His Ala Leu Pro His Val Gly Asp Ile Arg
cag ctt ggc ttt atg tgc ggt gca gag ctt gta cga tca aag gaa act
Gln Leu Gly Phe Met Cys Gly Ala Glu Leu Val Arg Ser Lys Glu Thr
aaa gaa cct tac ccg gct gat cgg cgg att gga tac aaa gtt tcc tta
Lys Glu Pro Tyr Pro Ala Asp Arg Arg Ile Gly Tyr Lys Val Ser Leu
aaa atg aga gag tta gga atg ctg aca aga ccg ctt ggg gac gtg att
Lys Met Arg Glu Leu Gly Met Leu Thr Arg Pro Leu Gly Asp Val Ile
gca ttt ctt cct cct ctt gcc agc aca gct gaa gag ctc tcg gaa atg
Ala Phe Leu Pro Pro Leu Ala Ser Thr Ala Glu Glu Leu Ser Glu Met
gtt gcc att atg aaa caa gcg atc cac gag gtt acg agc ctt gaa gat
Val Ala Ile Met Lys Gln Ala Ile His Glu Val Thr Ser Leu Glu Asp
tga
SEQ ID NO: 92
PRT - Bacillus subtilis
Met Thr His Asp Leu Ile Glu Lys Ser Lys Lys His Leu Trp Leu Pro
Phe Thr Gln Met Lys Asp Tyr Asp Glu Asn Pro Leu Ile Ile Glu Ser
Gly Thr Gly Ile Lys Val Lys Asp Ile Asn Gly Lys Glu Tyr Tyr Asp
Gly Phe Ser Ser Val Trp Leu Asn Val His Gly His Arg Lys Lys Glu
Leu Asp Asp Ala Ile Lys Lys Gln Leu Gly Lys Ile Ala His Ser Thr
Leu Leu Gly Met Thr Asn Val Pro Ala Thr Gln Leu Ala Glu Thr Leu
Ile Asp Ile Ser Pro Lys Lys Leu Thr Arg Val Phe Tyr Ser Asp Ser
Gly Ala Glu Ala Met Glu Ile Ala Leu Lys Met Ala Phe Gln Tyr Trp
Lys Asn Ile Gly Lys Pro Glu Lys Gln Lys Phe Ile Ala Met Lys Asn
Gly Tyr His Gly Asp Thr Ile Gly Ala Val Ser Val Gly Ser Ile Glu
Leu Phe His His Val Tyr Gly Pro Leu Met Phe Glu Ser Tyr Lys Ala
Pro Ile Pro Tyr Val Tyr Arg Ser Glu Ser Gly Asp Pro Asp Glu Cys
Arg Asp Gln Cys Leu Arg Glu Leu Ala Gln Leu Leu Glu Glu His His
Glu Glu Ile Ala Ala Leu Ser Ile Glu Ser Met Val Gln Gly Ala Ser
Gly Met Ile Val Met Pro Glu Gly Tyr Leu Ala Gly Val Arg Glu Leu
Cys Thr Thr Tyr Asp Val Leu Met Ile Val Asp Glu Val Ala Thr Gly
Phe Gly Arg Thr Gly Lys Met Phe Ala Cys Glu His Glu Asn Val Gln
Pro Asp Leu Met Ala Ala Gly Lys Gly Ile Thr Gly Gly Tyr Leu Pro
Ile Ala Val Thr Phe Ala Thr Glu Asp Ile Tyr Lys Ala Phe Tyr Asp
Asp Tyr Glu Asn Leu Lys Thr Phe Phe His Gly His Ser Tyr Thr Gly
Asn Gln Leu Gly Cys Ala Val Ala Leu Glu Asn Leu Ala Leu Phe Glu
Ser Glu Asn Ile Val Glu Gln Val Ala Glu Lys Ser Lys Lys Leu His
Phe Leu Leu Gln Asp Leu His Ala Leu Pro His Val Gly Asp Ile Arg
Gln Leu Gly Phe Met Cys Gly Ala Glu Leu Val Arg Ser Lys Glu Thr
Lys Glu Pro Tyr Pro Ala Asp Arg Arg Ile Gly Tyr Lys Val Ser Leu
Lys Met Arg Glu Leu Gly Met Leu Thr Arg Pro Leu Gly Asp Val Ile
Ala Phe Leu Pro Pro Leu Ala Ser Thr Ala Glu Glu Leu Ser Glu Met
Val Ala Ile Met Lys Gln Ala Ile His Glu Val Thr Ser Leu Glu Asp
SEQ ID NO: 93
DNA - Rhodobacter sphaeroides
atg ccc ggt tgc ggg ggc ttg ccc ggg aat gaa ccg aaa tgc gga cga
Met Pro Gly Cys Gly Gly Leu Pro Gly Asn Glu Pro Lys Cys Gly Arg
gag ggg agg tcg gcg atg acg cgg aat gac gcg acg aat gct gcc gga
Glu Gly Arg Ser Ala Met Thr Arg Asn Asp Ala Thr Asn Ala Ala Gly
gcg gtg ggc gcg gcg atg cgg gat cac atc ctc ttg cct gca cag gaa
Ala Val Gly Ala Ala Met Arg Asp His Ile Leu Leu Pro Ala Gln Glu
atg gcg aag ctc ggc aag tcc gcg cag ccg gtg ctg act cat gcc gag
Met Ala Lys Leu Gly Lys Ser Ala Gln Pro Val Leu Thr His Ala Glu
ggc atc tat gtc cat acc gag gac ggc cgc cgc ctg atc gac ggg ccg
Gly Ile Tyr Val His Thr Glu Asp Gly Arg Arg Leu Ile Asp Gly Pro
gcg ggc atg tgg tgc gcg cag gtg ggc tac ggc cgc cgc gag atc gtc
Ala Gly Met Trp Cys Ala Gln Val Gly Tyr Gly Arg Arg Glu Ile Val
gat gcc atg gcg cat cag gcg atg gtg ctg ccc tat gcc tcg ccc tgg
Asp Ala Met Ala His Gln Ala Met Val Leu Pro Tyr Ala Ser Pro Trp
tat atg gcc acg agc ccc gcg gcg cgg ctg gcg gag aag atc gcc acg
Tyr Met Ala Thr Ser Pro Ala Ala Arg Leu Ala Glu Lys Ile Ala Thr
ctg acg ccg ggc gat ctc aac cgg atc ttt ttc acc acg ggc ggg tcg
Leu Thr Pro Gly Asp Leu Asn Arg Ile Phe Phe Thr Thr Gly Gly Ser
acc gcg gtg gac agc gcg ctg cgc ttc tcg gaa ttc tac aac aac gtg
Thr Ala Val Asp Ser Ala Leu Arg Phe Ser Glu Phe Tyr Asn Asn Val
ctg ggc cgg ccg cag aag aag cgc atc atc gtg cgc tac gac ggc tat
Leu Gly Arg Pro Gln Lys Lys Arg Ile Ile Val Arg Tyr Asp Gly Tyr
cac ggc tcg acg gcg ctc acc gcc gcc tgc acc ggc cgc acc ggc aac
His Gly Ser Thr Ala Leu Thr Ala Ala Cys Thr Gly Arg Thr Gly Asn
tgg ccg aac ttc gac atc gcg cag gac cgg atc tcg ttc ctc tcg agc
Trp Pro Asn Phe Asp Ile Ala Gln Asp Arg Ile Ser Phe Leu Ser Ser
ccc aat ccg cgc cac gcc ggc aac cgc agc cag gag gcg ttc ctc gac
Pro Asn Pro Arg His Ala Gly Asn Arg Ser Gln Glu Ala Phe Leu Asp
gat ctg gtg cag gaa ttc gag gac cgg atc gag agc ctc ggc ccc gac
Asp Leu Val Gln Glu Phe Glu Asp Arg Ile Glu Ser Leu Gly Pro Asp
acg atc gcg gcc ttc ctg gcc gag ccg atc ctc gcc tcg ggc ggc gtc
Thr Ile Ala Ala Phe Leu Ala Glu Pro Ile Leu Ala Ser Gly Gly Val
att att ccg ccc gca ggc tat cat gcg cgc ttc aag gcg atc tgc gag
Ile Ile Pro Pro Ala Gly Tyr His Ala Arg Phe Lys Ala Ile Cys Glu
aag cac gac atc ctc tat atc tcg gac gag gtg gtg acg ggc ttc ggc
Lys His Asp Ile Leu Tyr Ile Ser Asp Glu Val Val Thr Gly Phe Gly
cgt tgc ggc gag tgg ttc gcc tcg gag aag gtg ttc ggg gtg gtg ccg
Arg Cys Gly Glu Trp Phe Ala Ser Glu Lys Val Phe Gly Val Val Pro
gac atc atc acc ttc gcc aag ggc gtg acc tcg ggc tat gtg ccg ctc
Asp Ile Ile Thr Phe Ala Lys Gly Val Thr Ser Gly Tyr Val Pro Leu
ggc ggc ctt gcg atc tcc gag gcg gtg ctg gcg cgg atc tcg ggc gag
Gly Gly Leu Ala Ile Ser Glu Ala Val Leu Ala Arg Ile Ser Gly Glu
aat gcc aag gga agc tgg ttc acc aac ggc tat acc tac agc aat cag
Asn Ala Lys Gly Ser Trp Phe Thr Asn Gly Tyr Thr Tyr Ser Asn Gln
ccg gtg gcc tgc gcc gcg gcg ctt gcc aac atc gag ctg atg gag cgc
Pro Val Ala Cys Ala Ala Ala Leu Ala Asn Ile Glu Leu Met Glu Arg
gag ggc atc gtc gat cag gcg cgc gag atg gcg gac tat ttc gcc gcg
Glu Gly Ile Val Asp Gln Ala Arg Glu Met Ala Asp Tyr Phe Ala Ala
gcg ctg gct tcg ctg cgc gat ctg ccg ggc gtg gcg gaa acc cgg tcg
Ala Leu Ala Ser Leu Arg Asp Leu Pro Gly Val Ala Glu Thr Arg Ser
gtg ggc ctc gtg ggt tgc gtg caa tgc ctg ctc gac ccg acc cgg gcg
Val Gly Leu Val Gly Cys Val Gln Cys Leu Leu Asp Pro Thr Arg Ala
gac ggc acg gcc gag gac aag gcc ttc acc ctg aag atc gac gag cgc
Asp Gly Thr Ala Glu Asp Lys Ala Phe Thr Leu Lys Ile Asp Glu Arg
tgc ttc gag ctc ggg ctg atc gtg cgc ccg ctg ggc gat ctc tgc gtg
Cys Phe Glu Leu Gly Leu Ile Val Arg Pro Leu Gly Asp Leu Cys Val
atc tcg ccg ccg ctc atc atc tcg cgc gcg cag atc gac gag atg gtc
Ile Ser Pro Pro Leu Ile Ile Ser Arg Ala Gln Ile Asp Glu Met Val
gcg atc atg cgg cag gcc atc acc gaa gtg agc gcc gcc cac ggt ctg
Ala Ile Met Arg Gln Ala Ile Thr Glu Val Ser Ala Ala His Gly Leu
acc gcg aaa gaa ccg gcc gcc gtc tga
Thr Ala Lys Glu Pro Ala Ala Val
SEQ ID NO: 94
PRT - Rhodobacter sphaeroides
Met Pro Gly Cys Gly Gly Leu Pro Gly Asn Glu Pro Lys Cys Gly Arg
Glu Gly Arg Ser Ala Met Thr Arg Asn Asp Ala Thr Asn Ala Ala Gly
Ala Val Gly Ala Ala Met Arg Asp His Ile Leu Leu Pro Ala Gln Glu
Met Ala Lys Leu Gly Lys Ser Ala Gln Pro Val Leu Thr His Ala Glu
Gly Ile Tyr Val His Thr Glu Asp Gly Arg Arg Leu Ile Asp Gly Pro
Ala Gly Met Trp Cys Ala Gln Val Gly Tyr Gly Arg Arg Glu Ile Val
Asp Ala Met Ala His Gln Ala Met Val Leu Pro Tyr Ala Ser Pro Trp
Tyr Met Ala Thr Ser Pro Ala Ala Arg Leu Ala Glu Lys Ile Ala Thr
Leu Thr Pro Gly Asp Leu Asn Arg Ile Phe Phe Thr Thr Gly Gly Ser
Thr Ala Val Asp Ser Ala Leu Arg Phe Ser Glu Phe Tyr Asn Asn Val
Leu Gly Arg Pro Gln Lys Lys Arg Ile Ile Val Arg Tyr Asp Gly Tyr
His Gly Ser Thr Ala Leu Thr Ala Ala Cys Thr Gly Arg Thr Gly Asn
Trp Pro Asn Phe Asp Ile Ala Gln Asp Arg Ile Ser Phe Leu Ser Ser
Pro Asn Pro Arg His Ala Gly Asn Arg Ser Gln Glu Ala Phe Leu Asp
Asp Leu Val Gln Glu Phe Glu Asp Arg Ile Glu Ser Leu Gly Pro Asp
Thr Ile Ala Ala Phe Leu Ala Glu Pro Ile Leu Ala Ser Gly Gly Val
Ile Ile Pro Pro Ala Gly Tyr His Ala Arg Phe Lys Ala Ile Cys Glu
Lys His Asp Ile Leu Tyr Ile Ser Asp Glu Val Val Thr Gly Phe Gly
Arg Cys Gly Glu Trp Phe Ala Ser Glu Lys Val Phe Gly Val Val Pro
Asp Ile Ile Thr Phe Ala Lys Gly Val Thr Ser Gly Tyr Val Pro Leu
Gly Gly Leu Ala Ile Ser Glu Ala Val Leu Ala Arg Ile Ser Gly Glu
Asn Ala Lys Gly Ser Trp Phe Thr Asn Gly Tyr Thr Tyr Ser Asn Gln
Pro Val Ala Cys Ala Ala Ala Leu Ala Asn Ile Glu Leu Met Glu Arg
Glu Gly Ile Val Asp Gln Ala Arg Glu Met Ala Asp Tyr Phe Ala Ala
Ala Leu Ala Ser Leu Arg Asp Leu Pro Gly Val Ala Glu Thr Arg Ser
Val Gly Leu Val Gly Cys Val Gln Cys Leu Leu Asp Pro Thr Arg Ala
Asp Gly Thr Ala Glu Asp Lys Ala Phe Thr Leu Lys Ile Asp Glu Arg
Cys Phe Glu Leu Gly Leu Ile Val Arg Pro Leu Gly Asp Leu Cys Val
Ile Ser Pro Pro Leu Ile Ile Ser Arg Ala Gln Ile Asp Glu Met Val
Ala Ile Met Arg Gln Ala Ile Thr Glu Val Ser Ala Ala His Gly Leu
Thr Ala Lys Glu Pro Ala Ala Val
SEQ ID NO: 95
DNA - Legionella pneumophila
atg agt atc gca ttt gtt aac ggc aag tat tgt tgt caa tct gaa gca
Met Ser Ile Ala Phe Val Asn Gly Lys Tyr Cys Cys Gln Ser Glu Ala
aaa att tca ata ttt gat cga ggg ttt ctt ttt ggt gac tcg gtt tat
Lys Ile Ser Ile Phe Asp Arg Gly Phe Leu Phe Gly Asp Ser Val Tyr
gaa gtg ctg cct gtt tac cat ggg cag cct tac ttt gta gac caa cat
Glu Val Leu Pro Val Tyr His Gly Gln Pro Tyr Phe Val Asp Gln His
ctt gac cga tta ttc tca aat atg aaa aaa att aag atg att ata cca
Leu Asp Arg Leu Phe Ser Asn Met Lys Lys Ile Lys Met Ile Ile Pro
aat tat gat tgg cat ggt tta att cat aga cta ata tca gaa aat aat
Asn Tyr Asp Trp His Gly Leu Ile His Arg Leu Ile Ser Glu Asn Asn
ggc ggt aat tta caa gta tat atc caa gtc aca cga ggg aat caa ggg
Gly Gly Asn Leu Gln Val Tyr Ile Gln Val Thr Arg Gly Asn Gln Gly
gtg cgc aag cat gat atc cct act tcc atc aca cct tct gtt atc gca
Val Arg Lys His Asp Ile Pro Thr Ser Ile Thr Pro Ser Val Ile Ala
ttc act atg cat aat cca ttt ccc acc ctc gaa gat aag gaa cag gga
Phe Thr Met His Asn Pro Phe Pro Thr Leu Glu Asp Lys Glu Gln Gly
atg tca gca aaa ctg gtt gaa gat ttt cgg tgg atg aga tgt gat ata
Met Ser Ala Lys Leu Val Glu Asp Phe Arg Trp Met Arg Cys Asp Ile
aaa act act tct tta att gcc aat ata tta ctg aat gat gag gct gta
Lys Thr Thr Ser Leu Ile Ala Asn Ile Leu Leu Asn Asp Glu Ala Val
tct gca gga ttc cac act gca att ctt gcc cgg aac ggt cta att aca
Ser Ala Gly Phe His Thr Ala Ile Leu Ala Arg Asn Gly Leu Ile Thr
gag gga agt agt acc aac gta ttt att gtc gca cag gat ggt gtt att
Glu Gly Ser Ser Thr Asn Val Phe Ile Val Ala Gln Asp Gly Val Ile
aag aca cca ccc atg aat aat ttc tgt tta cca gga att act cgg caa
Lys Thr Pro Pro Met Asn Asn Phe Cys Leu Pro Gly Ile Thr Arg Gln
gtt gtt att gaa ata att aaa aaa tta gat tta aag ttc aga gaa ata
Val Val Ile Glu Ile Ile Lys Lys Leu Asp Leu Lys Phe Arg Glu Ile
gaa att agc att tca gag ctt ttt tct gct cag gaa gtt tgg ata aca
Glu Ile Ser Ile Ser Glu Leu Phe Ser Ala Gln Glu Val Trp Ile Thr
agt acg aca aaa gaa gta ttc cct att aca aag att aat gac tct ttg
Ser Thr Thr Lys Glu Val Phe Pro Ile Thr Lys Ile Asn Asp Ser Leu
att aat ggc gga aaa gtt ggc gaa tat tgg cgg ata att aat gat tcc
Ile Asn Gly Gly Lys Val Gly Glu Tyr Trp Arg Ile Ile Asn Asp Ser
tac caa caa cta gta aac taa
Tyr Gln Gln Leu Val Asn
SEQ ID NO: 96
PRT - Legionella pneumophila
Met Ser Ile Ala Phe Val Asn Gly Lys Tyr Cys Cys Gln Ser Glu Ala
Lys Ile Ser Ile Phe Asp Arg Gly Phe Leu Phe Gly Asp Ser Val Tyr
Glu Val Leu Pro Val Tyr His Gly Gln Pro Tyr Phe Val Asp Gln His
Leu Asp Arg Leu Phe Ser Asn Met Lys Lys Ile Lys Met Ile Ile Pro
Asn Tyr Asp Trp His Gly Leu Ile His Arg Leu Ile Ser Glu Asn Asn
Gly Gly Asn Leu Gln Val Tyr Ile Gln Val Thr Arg Gly Asn Gln Gly
Val Arg Lys His Asp Ile Pro Thr Ser Ile Thr Pro Ser Val Ile Ala
Phe Thr Met His Asn Pro Phe Pro Thr Leu Glu Asp Lys Glu Gln Gly
Met Ser Ala Lys Leu Val Glu Asp Phe Arg Trp Met Arg Cys Asp Ile
Lys Thr Thr Ser Leu Ile Ala Asn Ile Leu Leu Asn Asp Glu Ala Val
Ser Ala Gly Phe His Thr Ala Ile Leu Ala Arg Asn Gly Leu Ile Thr
Glu Gly Ser Ser Thr Asn Val Phe Ile Val Ala Gln Asp Gly Val Ile
Lys Thr Pro Pro Met Asn Asn Phe Cys Leu Pro Gly Ile Thr Arg Gln
Val Val Ile Glu Ile Ile Lys Lys Leu Asp Leu Lys Phe Arg Glu Ile
Glu Ile Ser ILe Ser Glu Leu Phe Ser Ala Gln Glu Val Trp Ile Thr
Ser Thr Thr Lys Glu Val Phe Pro Ile Thr Lys Ile Asn Asp Ser Leu
Ile Asn Gly Gly Lys Val Gly Glu Tyr Trp Arg Ile Ile Asn Asp Ser
Tyr Gln Gln Leu Val Asn
SEQ ID NO: 97
DNA - Nitrosomonas europaea
atg att tac ctc aat ggc aaa ttt ctg ccg atg gaa cag gct acc gtt
Met Ile Tyr Leu Asn Gly Lys Phe Leu Pro Met Glu Gln Ala Thr Val
cca gtg ctg gat aga ggc ttc atc ttc ggt gat ggt gtc tat gaa gtc
Pro Val Leu Asp Arg Gly Phe Ile Phe Gly Asp Gly Val Tyr Glu Val
ata ccg gtt tat tca cgt aaa ccg ttc cgg ctg ggc gaa cat ctt tcc
Ile Pro Val Tyr Ser Arg Lys Pro Phe Arg Leu Gly Glu His Leu Ser
cgg ctg cag cac agt ctg gat ggc ata cgt ctc cag aat ccg cac act
Arg Leu Gln His Ser Leu Asp Gly Ile Arg Leu Gln Asn Pro His Thr
gaa gaa caa tgg gct ggt ctg atc gaa cgc atc atc gag ctg aat gaa
Glu Glu Gln Trp Ala Gly Leu Ile Glu Arg Ile Ile Glu Leu Asn Glu
ggt gat gat cag tac ctt tac ctg cac att aca cgc ggg gtg gca aaa
Gly Asp Asp Gln Tyr Leu Tyr Leu His Ile Thr Arg Gly Val Ala Lys
cgt gac cat gcc ttt cct cgc gaa gta acg ccc act gtc ttc atc atg
Arg Asp His Ala Phe Pro Arg Glu Val Thr Pro Thr Val Phe Ile Met
agc aac ccg ctt ccg gct cca cct gca aaa ttg ctc gtt tcc gga gtt
Ser Asn Pro Leu Pro Ala Pro Pro Ala Lys Leu Leu Val Ser Gly Val
tca gcg att acc gcc agg gat aat cgc tgg ggg cgc tgt gat atc aaa
Ser Ala Ile Thr Ala Arg Asp Asn Arg Trp Gly Arg Cys Asp Ile Lys
gcc att tca ctg ttg cca aat atc tta ttg cgc cag ctt gcc gtg gac
Ala Ile Ser Leu Leu Pro Asn Ile Leu Leu Arg Gln Leu Ala Val Asp
gca caa gcc atg gaa acg atc ctg tta cgc gat ggt ctg ttg acc gaa
Ala Gln Ala Met Glu Thr Ile Leu Leu Arg Asp Gly Leu Leu Thr Glu
ggg gcc gcc agc aat att ttc atc gta aaa gac gac ctg ctg ctg acc
Gly Ala Ala Ser Asn Ile Phe Ile Val Lys Asp Asp Leu Leu Leu Thr
ccc ccc aaa gat cac cgt ata ttg cct ggc att act tat gat gta gta
Pro Pro Lys Asp His Arg Ile Leu Pro Gly Ile Thr Tyr Asp Val Val
ctg gaa ctg gct gaa aca cat ggt gtt cca cat gcg aca aga gaa ata
Leu Glu Leu Ala Glu Thr His Gly Val Pro His Ala Thr Arg Glu Ile
tca gag ctt gag tta cgt act gca cgg gaa atc atg ctg act tct tcc
Ser Glu Leu Glu Leu Arg Thr Ala Arg Glu Ile Met Leu Thr Ser Ser
acc aaa gaa att ctc ccg atc aca cag ctg gat gga caa ccg atc ggt
Thr Lys Glu Ile Leu Pro Ile Thr Gln Leu Asp Gly Gln Pro Ile Gly
aat ggc acc cca ggg cca gta ttt cag caa ctg gat cgg ctc tat cag
Asn Gly Thr Pro Gly Pro Val Phe Gln Gln Leu Asp Arg Leu Tyr Gln
gca tat aag ctg gaa gtc atg cgc ggg cat gct cca cgc cag taa
Ala Tyr Lys Leu Glu Val Met Arg Gly His Ala Pro Arg Gln
SEQ ID NO: 98
PRT - Nitrosomonas europaea
Met Ile Tyr Leu Asn Gly Lys Phe Leu Pro Met Glu Gln Ala Thr Val
Pro Val Leu Asp Arg Gly Phe Ile Phe Gly Asp Gly Val Tyr Glu Val
Ile Pro Val Tyr Ser Arg Lys Pro Phe Arg Leu Gly Glu His Leu Ser
Arg Leu Gln His Ser Leu Asp Gly Ile Arg Leu Gln Asn Pro His Thr
Glu Glu Gln Trp Ala Gly Leu Ile Glu Arg Ile Ile Glu Leu Asn Glu
Gly Asp Asp Gln Tyr Leu Tyr Leu His Ile Thr Arg Gly Val Ala Lys
Arg Asp His Ala Phe Pro Arg Glu Val Thr Pro Thr Val Phe Ile Met
Ser Asn Pro Leu Pro Ala Pro Pro Ala Lys Leu Leu Val Ser Gly Val
Ser Ala Ile Thr Ala Arg Asp Asn Arg Trp Gly Arg Cys Asp Ile Lys
Ala Ile Ser Leu Leu Pro Asn Ile Leu Leu Arg Gln Leu Ala Val Asp
Ala Gln Ala Met Glu Thr Ile Leu Leu Arg Asp Gly Leu Leu Thr Glu
Gly Ala Ala Ser Asn Ile Phe Ile Val Lys Asp Asp Leu Leu Leu Thr
Pro Pro Lys Asp His Arg Ile Leu Pro Gly Ile Thr Tyr Asp Val Val
Leu Glu Leu Ala Glu Thr His Gly Val Pro His Ala Thr Arg Glu Ile
Ser Glu Leu Glu Leu Arg Thr Ala Arg Glu Ile Met Leu Thr Ser Ser
Thr Lys Glu Ile Leu Pro Ile Thr Gln Leu Asp Gly Gln Pro Ile Gly
Asn Gly Thr Pro Gly Pro Val Phe Gln Gln Leu Asp Arg Leu Tyr Gln
Ala Tyr Lys Leu Glu Val Met Arg Gly His Ala Pro Arg Gln
SEQ ID NO: 99
DNA - Neisseria gonorrhoeae
atg agg ata aat atg aac cgt aac gaa att tta ttc gac cgc gcc aag
Met Arg Ile Asn Met Asn Arg Asn Glu Ile Leu Phe Asp Arg Ala Lys
gcc atc atc ccc ggc ggc gtg aat tcg ccc gtg cgc gca ttc ggc agc
Ala Ile Ile Pro Gly Gly Val Asn Ser Pro Val Arg Ala Phe Gly Ser
gtc ggc ggc gtg ccg cgc ttc atc aaa aaa gcc gaa ggc gcg tat gtt
Val Gly Gly Val Pro Arg Phe Ile Lys Lys Ala Glu Gly Ala Tyr Val
tgg gac gaa aac ggc acg cgc tac acc gat tat gtc ggc tct tgg ggg
Trp Asp Glu Asn Gly Thr Arg Tyr Thr Asp Tyr Val Gly Ser Trp Gly
cct gcg att gtc gga cac gcg cat ccc gaa gtc gtc gaa gcc gtg cgc
Pro Ala Ile Val Gly His Ala His Pro Glu Val Val Glu Ala Val Arg
gaa gct gcg ttg ggc ggt ttg tcg ttc ggc gcg ccc acc gaa ggc gaa
Glu Ala Ala Leu Gly Gly Leu Ser Phe Gly Ala Pro Thr Glu Gly Glu
atc gcc att gcc gaa caa att gcc gaa att atg ccg tct gtc gaa cgg
Ile Ala Ile Ala Glu Gln Ile Ala Glu Ile Met Pro Ser Val Glu Arg
ctg cgc ctc gtc agc tcc ggc acg gaa gcg acg atg act gcc atc cgt
Leu Arg Leu Val Ser Ser Gly Thr Glu Ala Thr Met Thr Ala Ile Arg
ctg gca cgc ggt ttt acc ggc cgc gac aaa atc atc aaa ttt gaa ggc
Leu Ala Arg Gly Phe Thr Gly Arg Asp Lys Ile Ile Lys Phe Glu Gly
tgc tac cac ggc cat tcc gac agc ctg ttg gtg aaa gca ggc agc ggt
Cys Tyr His Gly His Ser Asp Ser Leu Leu Val Lys Ala Gly Ser Gly
ctg ctt acc ttc ggc aat cct tct tcc gcc ggt gtg cct gcc gac ttt
Leu Leu Thr Phe Gly Asn Pro Ser Ser Ala Gly Val Pro Ala Asp Phe
acc aaa cat act ttg gta ctc gaa tac aac aac atc gcc caa ctc gaa
Thr Lys His Thr Leu Val Leu Glu Tyr Asn Asn Ile Ala Gln Leu Glu
gaa gcc ttt gcc caa agc ggc gac gaa atc gcc tgc gtg att gtc gaa
Glu Ala Phe Ala Gln Ser Gly Asp Glu Ile Ala Cys Val Ile Val Glu
ccc ttc gtc ggc aat atg aac ctc gtc cgc ccg acc gaa gcc ttt gtc
Pro Phe Val Gly Asn Met Asn Leu Val Arg Pro Thr Glu Ala Phe Val
aaa gcc ttg cgc gga ttg acc gaa aaa cac ggc gcg gtg ttg att tac
Lys Ala Leu Arg Gly Leu Thr Glu Lys His Gly Ala Val Leu Ile Tyr
gac gaa gtg atg acc ggt ttc cgc gtc gcg ctc ggc ggc gcg cag tcg
Asp Glu Val Met Thr Gly Phe Arg Val Ala Leu Gly Gly Ala Gln Ser
ctg cac ggc atc acg ccc gac ctg acc acg atg ggc aaa gtc atc ggc
Leu His Gly Ile Thr Pro Asp Leu Thr Thr Met Gly Lys Val Ile Gly
ggc ggt atg ccg ctt gcc gcg ttc ggc gga cgc aaa gac atc atg gaa
Gly Gly Met Pro Leu Ala Ala Phe Gly Gly Arg Lys Asp Ile Met Glu
tgt att tcc ccg ttg ggc ggc gtg tat cag gca ggt aca tta tca ggc
Cys Ile Ser Pro Leu Gly Gly Val Tyr Gln Ala Gly Thr Leu Ser Gly
aac ccg att gcc gtc gcc gcc ggc ttg aaa acg ctg gaa atc atc cag
Asn Pro Ile Ala Val Ala Ala Gly Leu Lys Thr Leu Glu Ile Ile Gln
cgc gaa ggc ttc tat gaa aac ctg acc gcc ttg aca caa cgc ctt gcc
Arg Glu Gly Phe Tyr Glu Asn Leu Thr Ala Leu Thr Gln Arg Leu Ala
aac ggt att gcc gcc gcc aaa gcg cac ggt atc gag ttt gcc gcc gac
Asn Gly Ile Ala Ala Ala Lys Ala His Gly Ile Glu Phe Ala Ala Asp
agc gtg ggc ggt atg ttc ggt ctg tat ttc gcc gca cac gtg ccg cga
Ser Val Gly Gly Met Phe Gly Leu Tyr Phe Ala Ala His Val Pro Arg
aac tat gcc gat atg gcg cgc tcc aat atc gac gct ttc aaa cgc ttc
Asn Tyr Ala Asp Met Ala Arg Ser Asn Ile Asp Ala Phe Lys Arg Phe
ttc cac ggc atg ctc gac cgc ggc att gcc ttc ggc ccg tcc gct tat
Phe His Gly Met Leu Asp Arg Gly Ile Ala Phe Gly Pro Ser Ala Tyr
gaa gcg ggt ttc gtt tcc gcc gcg cat acg ccc gag ctg att gac gaa
Glu Ala Gly Phe Val Ser Ala Ala His Thr Pro Glu Leu Ile Asp Glu
acg gtt gcg gtt gcg gtt gaa gtg ttc aag gcg atg gct gca tga
Thr Val Ala Val Ala Val Glu Val Phe Lys Ala Met Ala Ala
SEQ ID NO: 100
PRT - Neisseria gonorrhoeae
Met Arg Ile Asn Met Asn Arg Asn Glu Ile Leu Phe Asp Arg Ala Lys
Ala Ile Ile Pro Gly Gly Val Asn Ser Pro Val Arg Ala Phe Gly Ser
Val Gly Gly Val Pro Arg Phe Ile Lys Lys Ala Glu Gly Ala Tyr Val
Trp Asp Glu Asn Gly Thr Arg Tyr Thr Asp Tyr Val Gly Ser Trp Gly
Pro Ala Ile Val Gly His Ala His Pro Glu Val Val Glu Ala Val Arg
Glu Ala Ala Leu Gly Gly Leu Ser Phe Gly Ala Pro Thr Glu Gly Glu
Ile Ala Ile Ala Glu Gln Ile Ala Glu Ile Met Pro Ser Val Glu Arg
Leu Arg Leu Val Ser Ser Gly Thr Glu Ala Thr Met Thr Ala Ile Arg
Leu Ala Arg Gly Phe Thr Gly Arg Asp Lys Ile Ile Lys Phe Glu Gly
Cys Tyr His Gly His Ser Asp Ser Leu Leu Val Lys Ala Gly Ser Gly
Leu Leu Thr Phe Gly Asn Pro Ser Ser Ala Gly Val Pro Ala Asp Phe
Thr Lys His Thr Leu Val Leu Glu Tyr Asn Asn Ile Ala Gln Leu Glu
Glu Ala Phe Ala Gln Ser Gly Asp Glu Ile Ala Cys Val Ile Val Glu
Pro Phe Val Gly Asn Met Asn Leu Val Arg Pro Thr Glu Ala Phe Val
Lys Ala Leu Arg Gly Leu Thr Glu Lys His Gly Ala Val Leu Ile Tyr
Asp Glu Val Met Thr Gly Phe Arg Val Ala Leu Gly Gly Ala Gln Ser
Leu His Gly Ile Thr Pro Asp Leu Thr Thr Met Gly Lys Val Ile Gly
Gly Gly Met Pro Leu Ala Ala Phe Gly Gly Arg Lys Asp Ile Met Glu
Cys Ile Ser Pro Leu Gly Gly Val Tyr Gln Ala Gly Thr Leu Ser Gly
Asn Pro Ile Ala Val Ala Ala Gly Leu Lys Thr Leu Glu Ile Ile Gln
Arg Glu Gly Phe Tyr Glu Asn Leu Thr Ala Leu Thr Gln Arg Leu Ala
Asn Gly Ile Ala Ala Ala Lys Ala His Gly Ile Glu Phe Ala Ala Asp
Ser Val Gly Gly Met Phe Gly Leu Tyr Phe Ala Ala His Val Pro Arg
Asn Tyr Ala Asp Met Ala Arg Ser Asn Ile Asp Ala Phe Lys Arg Phe
Phe His Gly Met Leu Asp Arg Gly Ile Ala Phe Gly Pro Ser Ala Tyr
Glu Ala Gly Phe Val Ser Ala Ala His Thr Pro Glu Leu Ile Asp Glu
Thr Val Ala Val Ala Val Glu Val Phe Lys Ala Met Ala Ala
SEQ ID NO: 101
DNA - Pseudomonas aeruginosa
atg tcg atg gcc gat cgt gat ggc gtg atc tgg tat gac ggt gaa ctg
Met Ser Met Ala Asp Arg Asp Gly Val Ile Trp Tyr Asp Gly Glu Leu
gtg cag tgg cgc gac gcg acc acg cac gtg ctg acc cat acc ctg cac
Val Gln Trp Arg Asp Ala Thr Thr His Val Leu Thr His Thr Leu His
tat gga atg ggc gtg ttc gag ggc gtg cgc gcc tac gac acc ccg cag
Tyr Gly Met Gly Val Phe Glu Gly Val Arg Ala Tyr Asp Thr Pro Gln
ggc acg gcg atc ttc cgc ctg cag gcg cat acc gac cgg ctg ttc gac
Gly Thr Ala Ile Phe Arg Leu Gln Ala His Thr Asp Arg Leu Phe Asp
tcc gcg cac atc atg aac atg cag atc ccg tac agc cgc gac gag atc
Ser Ala His Ile Met Asn Met Gln Ile Pro Tyr Ser Arg Asp Glu Ile
aac gag gcg acc cgc gcc gcc gtg cgc gag aac aac ctg gaa agc gcc
Asn Glu Ala Thr Arg Ala Ala Val Arg Glu Asn Asn Leu Glu Ser Ala
tat atc cgc ccg atg gtg ttc tac gga agc gaa ggc atg ggc ctg cgc
Tyr Ile Arg Pro Met Val Phe Tyr Gly Ser Glu Gly Met Gly Leu Arg
gcc agc ggc ctg aag gtc cat gtg atc atc gcc gcc tgg agc tgg ggc
Ala Ser Gly Leu Lys Val His Val Ile Ile Ala Ala Trp Ser Trp Gly
gcc tac atg ggc gag gaa gcc ctg cag caa ggc atc aag gtg cgc acc
Ala Tyr Met Gly Glu Glu Ala Leu Gln Gln Gly Ile Lys Val Arg Thr
agt tcc ttc acc cgc cac cac gtc aac atc tcg atg acc cgc gcc aag
Ser Ser Phe Thr Arg His His Val Asn Ile Ser Met Thr Arg Ala Lys
tcc aac ggc gcc tac atc aac tcg atg ctg gcc ctc cag gaa gcg atc
Ser Asn Gly Ala Tyr Ile Asn Ser Met Leu Ala Leu Gln Glu Ala Ile
tcc ggc ggc gcc gac gag gcc atg atg ctc gat ccg gaa ggc tac gtg
Ser Gly Gly Ala Asp Glu Ala Met Met Leu Asp Pro Glu Gly Tyr Val
gcc gaa ggc tcc ggc gag aac atc ttc atc atc aag gat ggc gtg atc
Ala Glu Gly Ser Gly Glu Asn Ile Phe Ile Ile Lys Asp Gly Val Ile
tac acc ccg gaa gtc acc gcc tgc ctg aac ggc atc act cgt aac act
Tyr Thr Pro Glu Val Thr Ala Cys Leu Asn Gly Ile Thr Arg Asn Thr
atc ctg acc ctg gcc gcc gaa cac ggt ttt aaa ctg gtc gag aag cgc
Ile Leu Thr Leu Ala Ala Glu His Gly Phe Lys Leu Val Glu Lys Arg
atc acc cgc gac gag gtg tac atc gcc gac gag gcc ttc ttc act ggc
Ile Thr Arg Asp Glu Val Tyr Ile Ala Asp Glu Ala Phe Phe Thr Gly
act gcc gcg gaa gtc acg ccg atc cgc gaa gtg gac ggt cgc aag atc
Thr Ala Ala Glu Val Thr Pro Ile Arg Glu Val Asp Gly Arg Lys Ile
ggc gcc ggc cgc cgt ggc ccg gtc acc gaa aag ctg cag aaa gcc tat
Gly Ala Gly Arg Arg Gly Pro Val Thr Glu Lys Leu Gln Lys Ala Tyr
ttc gac ctg gtc agc ggc aag acc gag gcc cac gcc gag tgg cgt acc
Phe Asp Leu Val Ser Gly Lys Thr Glu Ala His Ala Glu Trp Arg Thr
ctg gtc aag taa
Leu Val Lys
SEQ ID NO: 102
PRT - Pseudomonas aeruginosa
Met Ser Met Ala Asp Arg Asp Gly Val Ile Trp Tyr Asp Gly Glu Leu
Val Gln Trp Arg Asp Ala Thr Thr His Val Leu Thr His Thr Leu His
Tyr Gly Met Gly Val Phe Glu Gly Val Arg Ala Tyr Asp Thr Pro Gln
Gly Thr Ala Ile Phe Arg Leu Gln Ala His Thr Asp Arg Leu Phe Asp
Ser Ala His Ile Met Asn Met Gln Ile Pro Tyr Ser Arg Asp Glu Ile
Asn Glu Ala Thr Arg Ala Ala Val Arg Glu Asn Asn Leu Glu Ser Ala
Tyr Ile Arg Pro Met Val Phe Tyr Gly Ser Glu Gly Met Gly Leu Arg
Ala Ser Gly Leu Lys Val His Val Ile Ile Ala Ala Trp Ser Trp Gly
Ala Tyr Met Gly Glu Glu Ala Leu Gln Gln Gly Ile Lys Val Arg Thr
Ser Ser Phe Thr Arg His His Val Asn Ile Ser Met Thr Arg Ala Lys
Ser Asn Gly Ala Tyr Ile Asn Ser Met Leu Ala Leu Gln Glu Ala Ile
Ser Gly Gly Ala Asp Glu Ala Met Met Leu Asp Pro Glu Gly Tyr Val
Ala Glu Gly Ser Gly Glu Asn Ile Phe Ile Ile Lys Asp Gly Val Ile
Tyr Thr Pro Glu Val Thr Ala Cys Leu Asn Gly Ile Thr Arg Asn Thr
Ile Leu Thr Leu Ala Ala Glu His Gly Phe Lys Leu Val Glu Lys Arg
Ile Thr Arg Asp Glu Val Tyr Ile Ala Asp Glu Ala Phe Phe Thr Gly
Thr Ala Ala Glu Val Thr Pro Ile Arg Glu Val Asp Gly Arg Lys Ile
Gly Ala Gly Arg Arg Gly Pro Val Thr Glu Lys Leu Gln Lys Ala Tyr
Phe Asp Leu Val Ser Gly Lys Thr Glu Ala His Ala Glu Trp Arg Thr
Leu Val Lys
SEQ ID NO: 103
DNA - Rhodopseudomonas palustris
atg aag ctg ata ccg tgc cgc gcc ttt cac ccc ccg gcc gcg cag tgc
Met Lys Leu Ile Pro Cys Arg Ala Phe His Pro Pro Ala Ala Gln Cys
atg agg agc gcc atg tta gac aag atc aag ccc acg tcc gcc gtc aac
Met Arg Ser Ala Met Leu Asp Lys Ile Lys Pro Thr Ser Ala Val Asn
gcg ccg aac gat ctc aac gcg ttc tgg atg ccg ttc acc gcg aac cgg
Ala Pro Asn Asp Leu Asn Ala Phe Trp Met Pro Phe Thr Ala Asn Arg
gcc ttc aag cgc gcg ccg aag atg gtc gtg ggt gcc gaa ggc atg cac
Ala Phe Lys Arg Ala Pro Lys Met Val Val Gly Ala Glu Gly Met His
tac atc acc gcc gat ggt cgc aag atc atc gac gcc gcc tcg ggc atg
Tyr Ile Thr Ala Asp Gly Arg Lys Ile Ile Asp Ala Ala Ser Gly Met
tgg tgc acc aat gcg ggc cat ggc cgc aag gaa atc gcc gag gcg atc
Trp Cys Thr Asn Ala Gly His Gly Arg Lys Glu Ile Ala Glu Ala Ile
aag gcg cag gcc gat gaa ctc gac ttc tcg ccg ccg ttc cag ttc ggc
Lys Ala Gln Ala Asp Glu Leu Asp Phe Ser Pro Pro Phe Gln Phe Gly
cag ccg aag gcg ttc gaa ctc gcc agc cgg atc gcc gat ctg gcg ccg
Gln Pro Lys Ala Phe Glu Leu Ala Ser Arg Ile Ala Asp Leu Ala Pro
gaa ggc ctc gat cac gtg ttc ttc tgc aat tcg ggc tcg gaa gcc ggc
Glu Gly Leu Asp His Val Phe Phe Cys Asn Ser Gly Ser Glu Ala Gly
gac acc gcg ctg aag atc gcg gtc gcc tat cag cag atc aag ggc cag
Asp Thr Ala Leu Lys Ile Ala Val Ala Tyr Gln Gln Ile Lys Gly Gln
ggc tca cgc acc cgc ctg atc ggc cgc gag cgc ggc tat cac ggc gtc
Gly Ser Arg Thr Arg Leu Ile Gly Arg Glu Arg Gly Tyr His Gly Val
ggc ttc ggc ggc acc gcg gtc ggc ggc atc ggc aac aac cgc aag atg
Gly Phe Gly Gly Thr Ala Val Gly Gly Ile Gly Asn Asn Arg Lys Met
ttc ggt ccg ctg ctc aac ggc gtc gat cat ctg cct gcg act tat gat
Phe Gly Pro Leu Leu Asn Gly Val Asp His Leu Pro Ala Thr Tyr Asp
cgc gac aag cag gct ttc acc atc ggc gag ccg gaa tac ggc gcg cac
Arg Asp Lys Gln Ala Phe Thr Ile Gly Glu Pro Glu Tyr Gly Ala His
ttc gcc gaa gcg ctt gaa ggc ctc gtc aat ctg cac ggc gcc aac acc
Phe Ala Glu Ala Leu Glu Gly Leu Val Asn Leu His Gly Ala Asn Thr
atc gcg gcg gtg atc gtc gag ccg atg gcc ggc tcc acc ggc gtg ctg
Ile Ala Ala Val Ile Val Glu Pro Met Ala Gly Ser Thr Gly Val Leu
ccg gcg ccg aag ggc tat ctc aag aag ctg cgc gag atc acc aag aag
Pro Ala Pro Lys Gly Tyr Leu Lys Lys Leu Arg Glu Ile Thr Lys Lys
cac ggc atc ctg ctg atc ttc gac gag gtc atc acc ggc tac ggc cgt
His Gly Ile Leu Leu Ile Phe Asp Glu Val Ile Thr Gly Tyr Gly Arg
ctc ggc tat gcc ttc gcg tcc gaa cgt tac ggc gtc acc ccg gac atg
Leu Gly Tyr Ala Phe Ala Ser Glu Arg Tyr Gly Val Thr Pro Asp Met
atc acc ttc gcc aag ggc gtc acc aat ggt gcg gtg ccg atg ggc ggc
Ile Thr Phe Ala Lys Gly Val Thr Asn Gly Ala Val Pro Met Gly Gly
gtg atc acc tcg gcg gag atc cac gat gcg ttc atg acc ggc ccc gag
Val Ile Thr Ser Ala Glu Ile His Asp Ala Phe Met Thr Gly Pro Glu
cac gcg gtc gag ctg gcg cac ggc tac acc tat tcg gcg cat ccg ctc
His Ala Val Glu Leu Ala His Gly Tyr Thr Tyr Ser Ala His Pro Leu
gcc tgc gcg gcc ggc atc gcc acc ctc gac atc tac cgc gac gag aag
Ala Cys Ala Ala Gly Ile Ala Thr Leu Asp Ile Tyr Arg Asp Glu Lys
ctg ttc gag cgc gcc aag gcg ctg gag ccg aag ttt gcc gag gcg gtg
Leu Phe Glu Arg Ala Lys Ala Leu Glu Pro Lys Phe Ala Glu Ala Val
atg tcg ctg aag tcg gcc ccg aac gtg gtc gac atc cgc acc gtc ggc
Met Ser Leu Lys Ser Ala Pro Asn Val Val Asp Ile Arg Thr Val Gly
ctg acg gcg ggt atc gac ctc gct tcg atc gcc gat gcg gtc ggc aag
Leu Thr Ala Gly Ile Asp Leu Ala Ser Ile Ala Asp Ala Val Gly Lys
cgt ggc ttc gaa gcg atg aat gcc ggc ttc cac gac cac gag ctg atg
Arg Gly Phe Glu Ala Met Asn Ala Gly Phe His Asp His Glu Leu Met
ctg cgg atc gcc ggc gac acc ctg gcg ctg acc ccg ccg ctg atc ctc
Leu Arg Ile Ala Gly Asp Thr Leu Ala Leu Thr Pro Pro Leu Ile Leu
agc gag gac cac atc ggt gag atc gtc gac aag gtc ggc aag gtg atc
Ser Glu Asp His Ile Gly Glu Ile Val Asp Lys Val Gly Lys Val Ile
cgc gcg gtc gcc tga
Arg Ala Val Ala
SEQ ID NO: 104
PRT - Rhodopseudomonas palustris
Met Lys Leu Ile Pro Cys Arg Ala Phe His Pro Pro Ala Ala Gln Cys
Met Arg Ser Ala Met Leu Asp Lys Ile Lys Pro Thr Ser Ala Val Asn
Ala Pro Asn Asp Leu Asn Ala Phe Trp Met Pro Phe Thr Ala Asn Arg
Ala Phe Lys Arg Ala Pro Lys Met Val Val Gly Ala Glu Gly Met His
Tyr Ile Thr Ala Asp Gly Arg Lys Ile Ile Asp Ala Ala Ser Gly Met
Trp Cys Thr Asn Ala Gly His Gly Arg Lys Glu Ile Ala Glu Ala Ile
Lys Ala Gln Ala Asp Glu Leu Asp Phe Ser Pro Pro Phe Gln Phe Gly
Gln Pro Lys Ala Phe Glu Leu Ala Ser Arg Ile Ala Asp Leu Ala Pro
Glu Gly Leu Asp His Val Phe Phe Cys Asn Ser Gly Ser Glu Ala Gly
Asp Thr Ala Leu Lys Ile Ala Val Ala Tyr Gln Gln Ile Lys Gly Gln
Gly Ser Arg Thr Arg Leu Ile Gly Arg Glu Arg Gly Tyr His Gly Val
Gly Phe Gly Gly Thr Ala Val Gly Gly Ile Gly Asn Asn Arg Lys Met
Phe Gly Pro Leu Leu Asn Gly Val Asp His Leu Pro Ala Thr Tyr Asp
Arg Asp Lys Gln Ala Phe Thr Ile Gly Glu Pro Glu Tyr Gly Ala His
Phe Ala Glu Ala Leu Glu Gly Leu Val Asn Leu His Gly Ala Asn Thr
Ile Ala Ala Val Ile Val Glu Pro Met Ala Gly Ser Thr Gly Val Leu
Pro Ala Pro Lys Gly Tyr Leu Lys Lys Leu Arg Glu Ile Thr Lys Lys
His Gly Ile Leu Leu Ile Phe Asp Glu Val Ile Thr Gly Tyr Gly Arg
Leu Gly Tyr Ala Phe Ala Ser Glu Arg Tyr Gly Val Thr Pro Asp Met
Ile Thr Phe Ala Lys Gly Val Thr Asn Gly Ala Val Pro Met Gly Gly
Val Ile Thr Ser Ala Glu Ile His Asp Ala Phe Met Thr Gly Pro Glu
His Ala Val Glu Leu Ala His Gly Tyr Thr Tyr Ser Ala His Pro Leu
Ala Cys Ala Ala Gly Ile Ala Thr Leu Asp Ile Tyr Arg Asp Glu Lys
Leu Phe Glu Arg Ala Lys Ala Leu Glu Pro Lys Phe Ala Glu Ala Val
Met Ser Leu Lys Ser Ala Pro Asn Val Val Asp Ile Arg Thr Val Gly
Leu Thr Ala Gly Ile Asp Leu Ala Ser Ile Ala Asp Ala Val Gly Lys
Arg Gly Phe Glu Ala Met Asn Ala Gly Phe His Asp His Glu Leu Met
Leu Arg Ile Ala Gly Asp Thr Leu Ala Leu Thr Pro Pro Leu Ile Leu
Ser Glu Asp His Ile Gly Glu Ile Val Asp Lys Val Gly Lys Val Ile
Arg Ala Val Ala
SEQ ID NO: 105
DNA - Escherichia coli
atg cca cat tca ctg ttc agc acc gat acc gat ctc acc gcc gaa aat
Met Pro His Ser Leu Phe Ser Thr Asp Thr Asp Leu Thr Ala Glu Asn
ctg ctg cgt ttg ccc gct gaa ttt ggc tgc ccg gtg tgg gtc tac gat
Leu Leu Arg Leu Pro Ala Glu Phe Gly Cys Pro Val Trp Val Tyr Asp
gcg caa att att cgt cgg cag att gca gcg ctg aaa cag ttt gat gtg
Ala Gln Ile Ile Arg Arg Gln Ile Ala Ala Leu Lys Gln Phe Asp Val
gtg cgc ttt gca cag aaa gcc tgt tcc aat att cat att ttg cgc tta
Val Arg Phe Ala Gln Lys Ala Cys Ser Asn Ile His Ile Leu Arg Leu
atg cgt gag cag ggc gtg aaa gtg gat tcc gtc tcg tta ggc gaa ata
Met Arg Glu Gln Gly Val Lys Val Asp Ser Val Ser Leu Gly Glu Ile
gag cgt gcg ttg gcg gcg ggt tac aat ccg caa acg cac ccc gat gat
Glu Arg Ala Leu Ala Ala Gly Tyr Asn Pro Gln Thr His Pro Asp Asp
att gtt ttt acg gca gat gtt atc gat cag gcg acg ctt gaa cgc gtc
Ile Val Phe Thr Ala Asp Val Ile Asp Gln Ala Thr Leu Glu Arg Val
agt gaa ttg caa att ccg gtg aat gcg ggt tct gtt gat atg ctc gac
Ser Glu Leu Gln Ile Pro Val Asn Ala Gly Ser Val Asp Met Leu Asp
caa ctg ggc cag gtt tcg cca ggg cat cgg gta tgg ctg cgc gtt aat
Gln Leu Gly Gln Val Ser Pro Gly His Arg Val Trp Leu Arg Val Asn
ccg ggg ttt ggt cac gga cat agc caa aaa acc aat acc ggt ggc gaa
Pro Gly Phe Gly His Gly His Ser Gln Lys Thr Asn Thr Gly Gly Glu
aac agc aag cac ggt atc tgg tac acc gat ctg ccc gcc gca ctg gac
Asn Ser Lys His Gly Ile Trp Tyr Thr Asp Leu Pro Ala Ala Leu Asp
gtg ata caa cgt cat cat ctg cag ctg gtc ggc att cac atg cac att
Val Ile Gln Arg His His Leu Gln Leu Val Gly Ile His Met His Ile
ggt tct ggc gtt gat tat gcc cat ctg gaa cag gtg tgt ggt gct atg
Gly Ser Gly Val Asp Tyr Ala His Leu Glu Gln Val Cys Gly Ala Met
gtg cgt cag gtc atc gaa ttc ggt cag gat tta cag gct att tct gcg
Val Arg Gln Val Ile Glu Phe Gly Gln Asp Leu Gln Ala Ile Ser Ala
ggc ggt ggg ctt tct gtt cct tat caa cag ggt gaa gag gcg gtt gat
Gly Gly Gly Leu Ser Val Pro Tyr Gln Gln Gly Glu Glu Ala Val Asp
acc gaa cat tat tat ggt ctg tgg aat gcc gcg cgt gag caa atc gcc
Thr Glu His Tyr Tyr Gly Leu Trp Asn Ala Ala Arg Glu Gln Ile Ala
cgc cat ttg ggc cac cct gtg aaa ctg gaa att gaa ccg ggt cgc ttc
Arg His Leu Gly His Pro Val Lys Leu Glu Ile Glu Pro Gly Arg Phe
ctg gta gcg cag tct ggc gta tta att act cag gtg cgg agc gtc aaa
Leu Val Ala Gln Ser Gly Val Leu Ile Thr Gln Val Arg Ser Val Lys
caa atg ggg agc cgc cac ttt gtg ctg gtt gat gcc ggg ttc aac gat
Gln Met Gly Ser Arg His Phe Val Leu Val Asp Ala Gly Phe Asn Asp
ctg atg cgc ccg gca atg tac ggt agt tac cac cat atc agt gcc ctg
Leu Met Arg Pro Ala Met Tyr Gly Ser Tyr His His Ile Ser Ala Leu
gca gct gat ggt cgt tct ctg gaa cac gcg cca acg gtg gaa acc gtc
Ala Ala Asp Gly Arg Ser Leu Glu His Ala Pro Thr Val Glu Thr Val
gtc gcc gga ccg tta tgt gaa tcg ggc gat gtc ttt acc cag cag gaa
Val Ala Gly Pro Leu Cys Glu Ser Gly Asp Val Phe Thr Gln Gln Glu
ggg gga aat gtt gaa acc cgc gcc ttg ccg gaa gtg aag gca ggt gat
Gly Gly Asn Val Glu Thr Arg Ala Leu Pro Glu Val Lys Ala Gly Asp
tat ctg gta ctg cat gat aca ggg gca tat ggc gca tca atg tca tcc
Tyr Leu Val Leu His Asp Thr Gly Ala Tyr Gly Ala Ser Met Ser Ser
aac tac aat agc cgt ccg ctg tta cca gaa gtt ctg ttt gat aat ggt
Asn Tyr Asn Ser Arg Pro Leu Leu Pro Glu Val Leu Phe Asp Asn Gly
cag gcg cgg ttg att cgc cgt cgc cag acc atc gaa gaa tta ctg gcg
Gln Ala Arg Leu Ile Arg Arg Arg Gln Thr Ile Glu Glu Leu Leu Ala
ctg gaa ttg ctt taa
Leu Glu Leu Leu
SEQ ID NO: 106
PRT - Escherichia coli
Met Pro His Ser Leu Phe Ser Thr Asp Thr Asp Leu Thr Ala Glu Asn
Leu Leu Arg Leu Pro Ala Glu Phe Gly Cys Pro Val Trp Val Tyr Asp
Ala Gln Ile Ile Arg Arg Gln Ile Ala Ala Leu Lys Gln Phe Asp Val
Val Arg Phe Ala Gln Lys Ala Cys Ser Asn Ile His Ile Leu Arg Leu
Met Arg Glu Gln Gly Val Lys Val Asp Ser Val Ser Leu Gly Glu Ile
Glu Arg Ala Leu Ala Ala Gly Tyr Asn Pro Gln Thr His Pro Asp Asp
Ile Val Phe Thr Ala Asp Val Ile Asp Gln Ala Thr Leu Glu Arg Val
Ser Glu Leu Gln Ile Pro Val Asn Ala Gly Ser Val Asp Met Leu Asp
Gln Leu Gly Gln Val Ser Pro Gly His Arg Val Trp Leu Arg Val Asn
Pro Gly Phe Gly His Gly His Ser Gln Lys Thr Asn Thr Gly Gly Glu
Asn Ser Lys His Gly Ile Trp Tyr Thr Asp Leu Pro Ala Ala Leu Asp
Val Ile Gln Arg His His Leu Gln Leu Val Gly Ile His Met His Ile
Gly Ser Gly Val Asp Tyr Ala His Leu Glu Gln Val Cys Gly Ala Met
Val Arg Gln Val Ile Glu Phe Gly Gln Asp Leu Gln Ala Ile Ser Ala
Gly Gly Gly Leu Ser Val Pro Tyr Gln Gln Gly Glu Glu Ala Val Asp
Thr Glu His Tyr Tyr Gly Leu Trp Asn Ala Ala Arg Glu Gln Ile Ala
Arg His Leu Gly His Pro Val Lys Leu Glu Ile Glu Pro Gly Arg Phe
Leu Val Ala Gln Ser Gly Val Leu Ile Thr Gln Val Arg Ser Val Lys
Gln Met Gly Ser Arg His Phe Val Leu Val Asp Ala Gly Phe Asn Asp
Leu Met Arg Pro Ala Met Tyr Gly Ser Tyr His His Ile Ser Ala Leu
Ala Ala Asp Gly Arg Ser Leu Glu His Ala Pro Thr Val Glu Thr Val
Val Ala Gly Pro Leu Cys Glu Ser Gly Asp Val Phe Thr Gln Gln Glu
Gly Gly Asn Val Glu Thr Arg Ala Leu Pro Glu Val Lys Ala Gly Asp
Tyr Leu Val Leu His Asp Thr Gly Ala Tyr Gly Ala Ser Met Ser Ser
Asn Tyr Asn Ser Arg Pro Leu Leu Pro Glu Val Leu Phe Asp Asn Gly
Gln Ala Arg Leu Ile Arg Arg Arg Gln Thr Ile Glu Glu Leu Leu Ala
Leu Glu Leu Leu
SEQ ID NO: 107
DNA - Artificial
Escherichia.coli diaminopimelate decarboxylase LysA codon
optimised gene
atatgccaca ctctctgttt tctactgata ctgatctgac tgcggaaaac ctgctgcgtc
tgccggctga attcggttgt ccggtatggg tgtacgacgc tcagattatt cgtcgccaga
tcgcagcact gaagcagttc gatgtagtgc gttttgcaca gaaggcgtgc tccaacatcc
atatcctgcg cctgatgcgt gagcagggcg ttaaagttga ctccgtctct ctgggtgaga
ttgagcgcgc cctggcagcc ggctataacc cacagaccca tcctgacgac attgtattta
ctgccgacgt gatcgaccag gctactctgg aacgcgtttc tgaactgcag atcccggtta
atgctggttc tgtggacatg ctggaccagc tgggccaggt atccccaggt catcgtgtgt
ggctgcgtgt caacccaggt ttcggccacg gccactctca gaaaactaac actggtggtg
agaactccaa gcatggcatt tggtataccg atctgccggc tgcactggac gtaatccagc
gtcaccacct gcagctggtg ggcatccaca tgcacattgg ctccggcgta gactacgccc
acctggagca agtctgcggt gctatggtac gtcaggtaat cgagttcggc caagatctgc
aggcaatcag cgctggtggc ggcctgtctg taccttatca gcagggcgag gaggcggttg
acactgagca ctactacggt ctgtggaacg ccgctcgtga gcaaattgca cgtcacctgg
gccacccggt gaaactggag atcgagccgg gccgcttcct ggtagcacag tccggcgtac
tgattaccca ggtacgctct gttaaacaga tgggctcccg tcactttgtg ctggtagacg
caggcttcaa cgacctgatg cgtccggcta tgtatggttc ctatcatcac atctctgcgc
tggccgccga cggccgctct ctggaacacg cgccgacggt tgaaacggtg gtggctggtc
cgctgtgcga gtccggcgac gttttcactc agcaggaggg cggcaatgta gagacgcgtg
cgctgccgga agtgaaagcc ggtgattatc tggtgctgca tgataccggc gcctatggtg
cgagcatgag cagcaactac aactctcgcc cgctgctgcc ggaggtcctg ttcgataacg
gccaagcccg cctgatccgt cgtcgtcaga ccatcgagga actgctggca ctggagctgc
tgtaa
SEQ ID NO: 108
DNA - Saccharomyces cerevisiae
atg tct gaa att act ttg ggt aaa tat ttg ttc gaa aga tta aag caa
Met Ser Glu Ile Thr Leu Gly Lys Tyr Leu Phe Glu Arg Leu Lys Gln
gtc aac gtt aac acc gtt ttc ggt ttg cca ggt gac ttc aac ttg tcc
Val Asn Val Asn Thr Val Phe Gly Leu Pro Gly Asp Phe Asn Leu Ser
ttg ttg gac aag atc tac gaa gtt gaa ggt atg aga tgg gct ggt aac
Leu Leu Asp Lys Ile Tyr Glu Val Glu Gly Met Arg Trp Ala Gly Asn
gcc aac gaa ttg aac gct gct tac gcc gct gat ggt tac gct cgt atc
Ala Asn Glu Leu Asn Ala Ala Tyr Ala Ala Asp Gly Tyr Ala Arg Ile
aag ggt atg tct tgt atc atc acc acc ttc ggt gtc ggt gaa ttg tct
Lys Gly Met Ser Cys Ile Ile Thr Thr Phe Gly Val Gly Glu Leu Ser
gct ttg aac ggt att gcc ggt tct tac gct gaa cac gtc ggt gtt ttg
Ala Leu Asn Gly Ile Ala Gly Ser Tyr Ala Glu His Val Gly Val Leu
cac gtt gtt ggt gtc cca tcc atc tct gct caa gct aag caa ttg ttg
His Val Val Gly Val Pro Ser Ile Ser Ala Gln Ala Lys Gln Leu Leu
ttg cac cac acc ttg ggt aac ggt gac ttc act gtt ttc cac aga atg
Leu His His Thr Leu Gly Asn Gly Asp Phe Thr Val Phe His Arg Met
tct gcc aac att tct gaa acc act gct atg atc act gac att gct acc
Ser Ala Asn Ile Ser Glu Thr Thr Ala Met Ile Thr Asp Ile Ala Thr
gcc cca gct gaa att gac aga tgt atc aga acc act tac gtc acc caa
Ala Pro Ala Glu Ile Asp Arg Cys Ile Arg Thr Thr Tyr Val Thr Gln
aga cca gtc tac tta ggt ttg cca gct aac ttg gtc gac ttg aac gtc
Arg Pro Val Tyr Leu Gly Leu Pro Ala Asn Leu Val Asp Leu Asn Val
cca gct aag ttg ttg caa act cca att gac atg tct ttg aag cca aac
Pro Ala Lys Leu Leu Gln Thr Pro Ile Asp Met Ser Leu Lys Pro Asn
gat gct gaa tcc gaa aag gaa gtc att gac acc atc ttg gct ttg gtc
Asp Ala Glu Ser Glu Lys Glu Val Ile Asp Thr Ile Leu Ala Leu Val
aag gat gct aag aac cca gtt atc ttg gct gat gct tgt tgt tcc aga
Lys Asp Ala Lys Asn Pro Val Ile Leu Ala Asp Ala Cys Cys Ser Arg
cac gac gtc aag gct gaa act aag aag ttg att gac ttg act caa ttc
His Asp Val Lys Ala Glu Thr Lys Lys Leu Ile Asp Leu Thr Gln Phe
cca gct ttc gtc acc cca atg ggt aag ggt tcc att gac gaa caa cac
Pro Ala Phe Val Thr Pro Met Gly Lys Gly Ser Ile Asp Glu Gln His
cca aga tac ggt ggt gtt tac gtc ggt acc ttg tcc aag cca gaa gtt
Pro Arg Tyr Gly Gly Val Tyr Val Gly Thr Leu Ser Lys Pro Glu Val
aag gaa gcc gtt gaa tct gct gac ttg att ttg tct gtc ggt gct ttg
Lys Glu Ala Val Glu Ser Ala Asp Leu Ile Leu Ser Val Gly Ala Leu
ttg tct gat ttc aac acc ggt tct ttc tct tac tct tac aag acc aag
Leu Ser Asp Phe Asn Thr Gly Ser Phe Ser Tyr Ser Tyr Lys Thr Lys
aac att gtc gaa ttc cac tcc gac cac atg aag atc aga aac gcc act
Asn Ile Val Glu Phe His Ser Asp His Met Lys Ile Arg Asn Ala Thr
ttc cca ggt gtc caa atg aaa ttc gtt ttg caa aag ttg ttg acc act
Phe Pro Gly Val Gln Met Lys Phe Val Leu Gln Lys Leu Leu Thr Thr
att gct gac gcc gct aag ggt tac aag cca gtt gct gtc cca gct aga
Ile Ala Asp Ala Ala Lys Gly Tyr Lys Pro Val Ala Val Pro Ala Arg
act cca gct aac gct gct gtc cca gct tct acc cca ttg aag caa gaa
Thr Pro Ala Asn Ala Ala Val Pro Ala Ser Thr Pro Leu Lys Gln Glu
tgg atg tgg aac caa ttg ggt aac ttc ttg caa gaa ggt gat gtt gtc
Trp Met Trp Asn Gln Leu Gly Asn Phe Leu Gln Glu Gly Asp Val Val
att gct gaa acc ggt acc tcc gct ttc ggt atc aac caa acc act ttc
Ile Ala Glu Thr Gly Thr Ser Ala Phe Gly Ile Asn Gln Thr Thr Phe
cca aac aac acc tac ggt atc tct caa gtc tta tgg ggt tcc att ggt
Pro Asn Asn Thr Tyr Gly Ile Ser Gln Val Leu Trp Gly Ser Ile Gly
ttc acc act ggt gct acc ttg ggt gct gct ttc gct gct gaa gaa att
Phe Thr Thr Gly Ala Thr Leu Gly Ala Ala Phe Ala Ala Glu Glu Ile
gat cca aag aag aga gtt atc tta ttc att ggt gac ggt tct ttg caa
Asp Pro Lys Lys Arg Val Ile Leu Phe Ile Gly Asp Gly Ser Leu Gln
ttg act gtt caa gaa atc tcc acc atg atc aga tgg ggc ttg aag cca
Leu Thr Val Gln Glu Ile Ser Thr Met Ile Arg Trp Gly Leu Lys Pro
tac ttg ttc gtc ttg aac aac gat ggt tac acc att gaa aag ttg att
Tyr Leu Phe Val Leu Asn Asn Asp Gly Tyr Thr Ile Glu Lys Leu Ile
cac ggt cca aag gct caa tac aac gaa att caa ggt tgg gac cac cta
His Gly Pro Lys Ala Gln Tyr Asn Glu Ile Gln Gly Trp Asp His Leu
tcc ttg ttg cca act ttc ggt gct aag gac tat gaa acc cac aga gtc
Ser Leu Leu Pro Thr Phe Gly Ala Lys Asp Tyr Glu Thr His Arg Val
gct acc acc ggt gaa tgg gac aag ttg acc caa gac aag tct ttc aac
Ala Thr Thr Gly Glu Trp Asp Lys Leu Thr Gln Asp Lys Ser Phe Asn
gac aac tct aag atc aga atg att gaa atc atg ttg cca gtc ttc gat
Asp Asn Ser Lys Ile Arg Met Ile Glu Ile Met Leu Pro Val Phe Asp
gct cca caa aac ttg gtt gaa caa gct aag ttg act gct gct acc aac
Ala Pro Gln Asn Leu Val Glu Gln Ala Lys Leu Thr Ala Ala Thr Asn
gct aag caa taa
Ala Lys Gln
SEQ ID NO: 109
PRT - Saccharomyces cerevisiae
Met Ser Glu Ile Thr Leu Gly Lys Tyr Leu Phe Glu Arg Leu Lys Gln
Val Asn Val Asn Thr Val Phe Gly Leu Pro Gly Asp Phe Asn Leu Ser
Leu Leu Asp Lys Ile Tyr Glu Val Glu Gly Met Arg Trp Ala Gly Asn
Ala Asn Glu Leu Asn Ala Ala Tyr Ala Ala Asp Gly Tyr Ala Arg Ile
Lys Gly Met Ser Cys Ile Ile Thr Thr Phe Gly Val Gly Glu Leu Ser
Ala Leu Asn Gly Ile Ala Gly Ser Tyr Ala Glu His Val Gly Val Leu
His Val Val Gly Val Pro Ser Ile Ser Ala Gln Ala Lys Gln Leu Leu
Leu His His Thr Leu Gly Asn Gly Asp Phe Thr Val Phe His Arg Met
Ser Ala Asn Ile Ser Glu Thr Thr Ala Met Ile Thr Asp Ile Ala Thr
Ala Pro Ala Glu Ile Asp Arg Cys Ile Arg Thr Thr Tyr Val Thr Gln
Arg Pro Val Tyr Leu Gly Leu Pro Ala Asn Leu Val Asp Leu Asn Val
Pro Ala Lys Leu Leu Gln Thr Pro Ile Asp Met Ser Leu Lys Pro Asn
Asp Ala Glu Ser Glu Lys Glu Val Ile Asp Thr Ile Leu Ala Leu Val
Lys Asp Ala Lys Asn Pro Val Ile Leu Ala Asp Ala Cys Cys Ser Arg
His Asp Val Lys Ala Glu Thr Lys Lys Leu Ile Asp Leu Thr Gln Phe
Pro Ala Phe Val Thr Pro Met Gly Lys Gly Ser Ile Asp Glu Gln His
Pro Arg Tyr Gly Gly Val Tyr Val Gly Thr Leu Ser Lys Pro Glu Val
Lys Glu Ala Val Glu Ser Ala Asp Leu Ile Leu Ser Val Gly Ala Leu
Leu Ser Asp Phe Asn Thr Gly Ser Phe Ser Tyr Ser Tyr Lys Thr Lys
Asn Ile Val Glu Phe His Ser Asp His Met Lys Ile Arg Asn Ala Thr
Phe Pro Gly Val Gln Met Lys Phe Val Leu Gln Lys Leu Leu Thr Thr
Ile Ala Asp Ala Ala Lys Gly Tyr Lys Pro Val Ala Val Pro Ala Arg
Thr Pro Ala Asn Ala Ala Val Pro Ala Ser Thr Pro Leu Lys Gln Glu
Trp Met Trp Asn Gln Leu Gly Asn Phe Leu Gln Glu Gly Asp Val Val
Ile Ala Glu Thr Gly Thr Ser Ala Phe Gly Ile Asn Gln Thr Thr Phe
Pro Asn Asn Thr Tyr Gly Ile Ser Gln Val Leu Trp Gly Ser Ile Gly
Phe Thr Thr Gly Ala Thr Leu Gly Ala Ala Phe Ala Ala Glu Glu Ile
Asp Pro Lys Lys Arg Val Ile Leu Phe Ile Gly Asp Gly Ser Leu Gln
Leu Thr Val Gln Glu Ile Ser Thr Met Ile Arg Trp Gly Leu Lys Pro
Tyr Leu Phe Val Leu Asn Asn Asp Gly Tyr Thr Ile Glu Lys Leu Ile
His Gly Pro Lys Ala Gln Tyr Asn Glu Ile Gln Gly Trp Asp His Leu
Ser Leu Leu Pro Thr Phe Gly Ala Lys Asp Tyr Glu Thr His Arg Val
Ala Thr Thr Gly Glu Trp Asp Lys Leu Thr Gln Asp Lys Ser Phe Asn
Asp Asn Ser Lys Ile Arg Met Ile Glu Ile Met Leu Pro Val Phe Asp
Ala Pro Gln Asn Leu Val Glu Gln Ala Lys Leu Thr Ala Ala Thr Asn
Ala Lys Gln
SEQ ID NO: 110
DNA - Artificial
Saccharomyces cerevisiae pyruvate decarboxylase Pdc codon
optimised gene
atgtccgaga tcactctggg caaatacctg tttgaacgtc tgaaacaggt gaacgttaat
accgtattcg gcctgccggg tgatttcaac ctgtccctgc tggacaaaat ctatgaagtt
gaaggtatgc gttgggctgg caacgctaac gagctgaacg cagcgtacgc ggcagatggt
tacgctcgta tcaaaggtat gtcttgtatc atcaccacct tcggtgttgg tgagctgagc
gccctgaacg gcatcgccgg ctcctatgca gagcacgtgg gcgtgctgca cgttgtgggt
gtaccgtcca tcagcgccca ggcaaaacag ctgctgctgc accacaccct gggtaacggc
gactttaccg ttttccatcg tatgtctgcg aacatcagcg aaactactgc aatgattact
gacatcgcta cggcaccggc agaaatcgac cgttgcattc gtaccacgta cgttactcag
cgcccggttt atctgggcct gccagccaac ctggtggatc tgaacgtccc ggctaaactg
ctgcagactc cgatcgatat gtctctgaaa cctaacgacg cagaatctga gaaagaagtt
atcgatacta ttctggctct ggtgaaagat gcaaagaacc cagttatcct ggctgacgca
tgttgctctc gtcatgatgt aaaggcagaa accaaaaagc tgatcgacct gacgcagttc
ccggcgttcg ttaccccgat gggcaagggt tccatcgatg agcagcaccc gcgttatggt
ggtgtatacg ttggcacgct gtccaaaccg gaggtaaaag aagcggttga aagcgcagat
ctgatcctgt ctgttggtgc actgctgagc gacttcaaca ccggttcttt ctcctatagc
tacaagacca aaaacattgt ggagtttcac tccgatcaca tgaaaatccg caacgcgacc
tttcctggtg tgcagatgaa attcgtactg cagaaactgc tgaccaccat cgccgacgct
gcgaaaggtt ataaaccggt agctgtgccg gcacgtaccc cggcgaacgc cgcggttcct
gcatccactc cactgaagca ggaatggatg tggaatcagc tgggtaattt cctgcaagaa
ggcgacgttg taatcgcaga aaccggcact agcgcgtttg gcattaacca gacgaccttc
ccaaacaaca cctacggtat cagccaagtc ctgtggggct ctatcggctt caccaccggt
gcaaccctgg gtgcggcttt cgctgctgag gagatcgacc cgaagaaacg tgttatcctg
ttcatcggtg acggctccct gcagctgacc gtccaggaga tttctaccat gatccgctgg
ggcctgaaac cgtacctgtt tgtgctgaac aacgacggct acactattga gaaactgatc
cacggtccga aagcacagta taatgagatc cagggttggg atcatctgtc tctgctgccg
acctttggcg ctaaagacta cgagacccac cgcgtggcta ccaccggcga gtgggataaa
ctgacgcagg ataaatcctt caatgacaat agcaagattc gtatgatcga aatcatgctg
ccggtctttg atgctccgca gaacctggta gagcaagcaa aactgaccgc ggcaactaac
gctaaacagt aa
SEQ ID NO: 111
DNA - Zymomonas mobilis
atg agt tat act gtc ggt acc tat tta gcg gag cgg ctt gtc cag att
Met Ser Tyr Thr Val Gly Thr Tyr Leu Ala Glu Arg Leu Val Gln Ile
ggt ctc aag cat cac ttc gca gtc gcg ggc gac tac aac ctc gtc ctt
Gly Leu Lys His His Phe Ala Val Ala Gly Asp Tyr Asn Leu Val Leu
ctt gac aac ctg ctt ttg aac aaa aac atg gag cag gtt tat tgc tgt
Leu Asp Asn Leu Leu Leu Asn Lys Asn Met Glu Gln Val Tyr Cys Cys
aac gaa ctg aac tgc ggt ttc agt gca gaa ggt tat gct cgt gcc aaa
Asn Glu Leu Asn Cys Gly Phe Ser Ala Glu Gly Tyr Ala Arg Ala Lys
ggc gca gca gca gcc gtc gtt acc tac agc gtc ggt gcg ctt tcc gca
Gly Ala Ala Ala Ala Val Val Thr Tyr Ser Val Gly Ala Leu Ser Ala
ttt gat gct atc ggt ggc gcc tat gca gaa aac ctt ccg gtt atc ctg
Phe Asp Ala Ile Gly Gly Ala Tyr Ala Glu Asn Leu Pro Val Ile Leu
atc tcc ggt gct ccg aac aac aat gat cac gct gct ggt cac gtg ttg
Ile Ser Gly Ala Pro Asn Asn Asn Asp His Ala Ala Gly His Val Leu
cat cac gct ctt ggc aaa acc gac tat cac tat cag ttg gaa atg gcc
His His Ala Leu Gly Lys Thr Asp Tyr His Tyr Gln Leu Glu Met Ala
aag aac atc acg gcc gcc gct gaa gcg att tac acc ccg gaa gaa gct
Lys Asn Ile Thr Ala Ala Ala Glu Ala Ile Tyr Thr Pro Glu Glu Ala
ccg gct aaa atc gat cac gtg att aaa act gct ctt cgt gag aag aag
Pro Ala Lys Ile Asp His Val Ile Lys Thr Ala Leu Arg Glu Lys Lys
ccg gtt tat ctc gaa atc gct tgc aac att gct tcc atg ccc tgc gcc
Pro Val Tyr Leu Glu Ile Ala Cys Asn Ile Ala Ser Met Pro Cys Ala
gct cct gga ccg gca agc gca ttg ttc aat gac gaa gcc agc gac gaa
Ala Pro Gly Pro Ala Ser Ala Leu Phe Asn Asp Glu Ala Ser Asp Glu
gct tct ttg aat gca gcg gtt gaa gaa acc ctg aaa ttc atc gcc aac
Ala Ser Leu Asn Ala Ala Val Glu Glu Thr Leu Lys Phe Ile Ala Asn
cgc gac aaa gtt gcc gtc ctc gtc ggc agc aag ctg cgc gca gct ggt
Arg Asp Lys Val Ala Val Leu Val Gly Ser Lys Leu Arg Ala Ala Gly
gct gaa gaa gct gct gtc aaa ttt gct gat gct ctc ggt ggc gca gtt
Ala Glu Glu Ala Ala Val Lys Phe Ala Asp Ala Leu Gly Gly Ala Val
gct acc atg gct gct gca aaa agc ttc ttc cca gaa gaa aac ccg cat
Ala Thr Met Ala Ala Ala Lys Ser Phe Phe Pro Glu Glu Asn Pro His
tac atc ggc acc tca tgg ggt gaa gtc agc tat ccg ggc gtt gaa aag
Tyr Ile Gly Thr Ser Trp Gly Glu Val Ser Tyr Pro Gly Val Glu Lys
acg atg aaa gaa gcc gat gcg gtt atc gct ctg gct cct gtc ttc aac
Thr Met Lys Glu Ala Asp Ala Val Ile Ala Leu Ala Pro Val Phe Asn
gac tac tcc acc act ggt tgg acg gat att cct gat cct aag aaa ctg
Asp Tyr Ser Thr Thr Gly Trp Thr Asp Ile Pro Asp Pro Lys Lys Leu
gtt ctc gct gaa ccg cgt tct gtc gtc gtt aac ggc att cgc ttc ccc
Val Leu Ala Glu Pro Arg Ser Val Val Val Asn Gly Ile Arg Phe Pro
agc gtc cat ctg aaa gac tat ctg acc cgt ttg gct cag aaa gtt tcc
Ser Val His Leu Lys Asp Tyr Leu Thr Arg Leu Ala Gln Lys Val Ser
aag aaa acc ggt gca ttg gac ttc ttc aaa tcc ctc aat gca ggt gaa
Lys Lys Thr Gly Ala Leu Asp Phe Phe Lys Ser Leu Asn Ala Gly Glu
ctg aag aaa gcc gct ccg gct gat ccg agt gct ccg ttg gtc aac gca
Leu Lys Lys Ala Ala Pro Ala Asp Pro Ser Ala Pro Leu Val Asn Ala
gaa atc gcc cgt cag gtc gaa gct ctt ctg acc ccg aac acg acg gtt
Glu Ile Ala Arg Gln Val Glu Ala Leu Leu Thr Pro Asn Thr Thr Val
att gct gaa acc ggt gac tct tgg ttc aat gct cag cgc atg aag ctc
Ile Ala Glu Thr Gly Asp Ser Trp Phe Asn Ala Gln Arg Met Lys Leu
ccg aac ggt gct cgc gtt gaa tat gaa atg cag tgg ggt cac att ggt
Pro Asn Gly Ala Arg Val Glu Tyr Glu Met Gln Trp Gly His Ile Gly
tgg tcc gtt cct gcc gcc ttc ggt tat gcc gtc ggt gct ccg gaa cgt
Trp Ser Val Pro Ala Ala Phe Gly Tyr Ala Val Gly Ala Pro Glu Arg
cgc aac atc ctc atg gtt ggt gat ggt tcc ttc cag ctg acg gct cag
Arg Asn Ile Leu Met Val Gly Asp Gly Ser Phe Gln Leu Thr Ala Gln
gaa gtc gct cag atg gtt cgc ctg aaa ctg ccg gtt atc atc ttc ttg
Glu Val Ala Gln Met Val Arg Leu Lys Leu Pro Val Ile Ile Phe Leu
atc aat aac tat ggt tac acc gcc gaa gtt atg atc cat gat ggt ccg
Ile Asn Asn Tyr Gly Tyr Thr Ala Glu Val Met Ile His Asp Gly Pro
tac aac aac atc aag aac tgg gat tat gcc ggt ctg atg gaa gtg ttc
Tyr Asn Asn Ile Lys Asn Trp Asp Tyr Ala Gly Leu Met Glu Val Phe
aac ggt aac ggt ggt tat gac agc ggt gct ggt aaa ggc ctg aag gct
Asn Gly Asn Gly Gly Tyr Asp Ser Gly Ala Gly Lys Gly Leu Lys Ala
aaa acc ggt ggc gaa ctg gca gaa gct atc aag gtt gct ctg gca aac
Lys Thr Gly Gly Glu Leu Ala Glu Ala Ile Lys Val Ala Leu Ala Asn
acc gac ggc cca acc ctg atc gaa tgc ttc atc ggt cgt gaa gac tgc
Thr Asp Gly Pro Thr Leu Ile Glu Cys Phe Ile Gly Arg Glu Asp Cys
act gaa gaa ttg gtc aaa tgg ggt aag cgc gtt gct gcc gcc aac agc
Thr Glu Glu Leu Val Lys Trp Gly Lys Arg Val Ala Ala Ala Asn Ser
cgt aag cct gtt aac aag ctc ctc tag
Arg Lys Pro Val Asn Lys Leu Leu
SEQ ID NO: 112
PRT - Zymomonas mobilis
Met Ser Tyr Thr Val Gly Thr Tyr Leu Ala Glu Arg Leu Val Gln Ile
Gly Leu Lys His His Phe Ala Val Ala Gly Asp Tyr Asn Leu Val Leu
Leu Asp Asn Leu Leu Leu Asn Lys Asn Met Glu Gln Val Tyr Cys Cys
Asn Glu Leu Asn Cys Gly Phe Ser Ala Glu Gly Tyr Ala Arg Ala Lys
Gly Ala Ala Ala Ala Val Val Thr Tyr Ser Val Gly Ala Leu Ser Ala
Phe Asp Ala Ile Gly Gly Ala Tyr Ala Glu Asn Leu Pro Val Ile Leu
Ile Ser Gly Ala Pro Asn Asn Asn Asp His Ala Ala Gly His Val Leu
His His Ala Leu Gly Lys Thr Asp Tyr His Tyr Gln Leu Glu Met Ala
Lys Asn Ile Thr Ala Ala Ala Glu Ala Ile Tyr Thr Pro Glu Glu Ala
Pro Ala Lys Ile Asp His Val Ile Lys Thr Ala Leu Arg Glu Lys Lys
Pro Val Tyr Leu Glu Ile Ala Cys Asn Ile Ala Ser Met Pro Cys Ala
Ala Pro Gly Pro Ala Ser Ala Leu Phe Asn Asp Glu Ala Ser Asp Glu
Ala Ser Leu Asn Ala Ala Val Glu Glu Thr Leu Lys Phe Ile Ala Asn
Arg Asp Lys Val Ala Val Leu Val Gly Ser Lys Leu Arg Ala Ala Gly
Ala Glu Glu Ala Ala Val Lys Phe Ala Asp Ala Leu Gly Gly Ala Val
Ala Thr Met Ala Ala Ala Lys Ser Phe Phe Pro Glu Glu Asn Pro His
Tyr Ile Gly Thr Ser Trp Gly Glu Val Ser Tyr Pro Gly Val Glu Lys
Thr Met Lys Glu Ala Asp Ala Val Ile Ala Leu Ala Pro Val Phe Asn
Asp Tyr Ser Thr Thr Gly Trp Thr Asp Ile Pro Asp Pro Lys Lys Leu
Val Leu Ala Glu Pro Arg Ser Val Val Val Asn Gly Ile Arg Phe Pro
Ser Val His Leu Lys Asp Tyr Leu Thr Arg Leu Ala Gln Lys Val Ser
Lys Lys Thr Gly Ala Leu Asp Phe Phe Lys Ser Leu Asn Ala Gly Glu
Leu Lys Lys Ala Ala Pro Ala Asp Pro Ser Ala Pro Leu Val Asn Ala
Glu Ile Ala Arg Gln Val Glu Ala Leu Leu Thr Pro Asn Thr Thr Val
Ile Ala Glu Thr Gly Asp Ser Trp Phe Asn Ala Gln Arg Met Lys Leu
Pro Asn Gly Ala Arg Val Glu Tyr Glu Met Gln Trp Gly His Ile Gly
Trp Ser Val Pro Ala Ala Phe Gly Tyr Ala Val Gly Ala Pro Glu Arg
Arg Asn Ile Leu Met Val Gly Asp Gly Ser Phe Gln Leu Thr Ala Gln
Glu Val Ala Gln Met Val Arg Leu Lys Leu Pro Val Ile Ile Phe Leu
Ile Asn Asn Tyr Gly Tyr Thr Ala Glu Val Met Ile His Asp Gly Pro
Tyr Asn Asn Ile Lys Asn Trp Asp Tyr Ala Gly Leu Met Glu Val Phe
Asn Gly Asn Gly Gly Tyr Asp Ser Gly Ala Gly Lys Gly Leu Lys Ala
Lys Thr Gly Gly Glu Leu Ala Glu Ala Ile Lys Val Ala Leu Ala Asn
Thr Asp Gly Pro Thr Leu Ile Glu Cys Phe Ile Gly Arg Glu Asp Cys
Thr Glu Glu Leu Val Lys Trp Gly Lys Arg Val Ala Ala Ala Asn Ser
Arg Lys Pro Val Asn Lys Leu Leu
SEQ ID NO: 113
DNA - Artificial
Zymomonas mobilis pyruvate decarboxylase Pdcl472A codon optimised
gene
atgtcttata ctgttggtac ttatctggct gagcgtctgg tgcaaatcgg cctgaaacac
cactttgcag ttgctggcga ctacaacctg gttctgctgg ataacctgct gctgaacaaa
aacatggagc aagtttattg ctgtaacgag ctgaactgcg gcttctctgc ggagggttat
gcgcgtgcga aaggtgccgc tgcagcagtc gtaacctact ctgtgggcgc tctgtccgcg
ttcgacgcaa tcggtggcgc ttacgctgaa aacctgccgg tgatcctgat tagcggtgcg
ccgaataata acgaccatgc tgctggccac gttctgcacc acgccctggg taaaactgat
taccattacc agctggagat ggctaaaaac atcactgcag cagcagaagc gatctacacc
ccggaagagg ctccggcaaa aatcgaccac gtgattaaaa ccgctctgcg tgagaaaaag
ccggtatacc tggaaatcgc gtgcaacatc gcgtctatgc cgtgcgccgc accgggtccg
gcttctgccc tgttcaacga tgaggcgagc gatgaggcat ctctgaacgc agcagtagaa
gaaaccctga aatttatcgc aaaccgtgac aaagtagcag tcctggtagg ttctaaactg
cgtgcggctg gtgcggaaga ggctgcggta aagttcgcgg atgctctggg cggtgcagtg
gcgaccatgg cagcggctaa atccttcttc ccagaggaga acccgcatta cattggtacc
tcctggggcg aagtttccta ccctggtgtg gagaaaacca tgaaagaagc cgatgctgtg
attgccctgg cgcctgtatt caacgattat tccaccaccg gttggaccga tatcccggac
ccgaagaaac tggtcctggc tgaaccgcgc tccgtagtag tgaatggcat tcgtttcccg
tccgtacacc tgaaggatta cctgacgcgt ctggcacaga aagtatccaa gaaaactggc
gcgctggact tctttaaatc cctgaacgct ggtgagctga aaaaggcggc tccggccgat
ccgtccgcac cgctggtgaa cgcagagatt gcacgtcagg ttgaggcact gctgacgccg
aacaccaccg taatcgcgga aacgggcgac tcttggttca acgcacagcg catgaaactg
ccgaacggtg cccgcgttga atatgaaatg cagtggggtc acatcggctg gtctgtccca
gcagcgtttg gttacgcggt tggtgcaccg gagcgtcgca acatcctgat ggtgggtgac
ggctccttcc agctgactgc tcaggaggtg gcgcagatgg tgcgcctgaa gctgccggtt
atcattttcc tgatcaacaa ctacggctac accgccgagg taatgatcca cgatggtccg
tacaacaaca tcaaaaactg ggactacgcc ggtctgatgg aggtttttaa cggtaacggc
ggttacgaca gcggtgctgg taagggtctg aaagccaaaa ccggtggcga actggcagag
gcgattaaag ttgcgctggc aaacaccgat ggcccgaccc tgatcgagtg cttcatcggc
cgtgaggact gcaccgagga gctggtcaaa tggggcaaac gtgtggcggc tgctaactct
cgcaagccgg taaacaaact gctgtaa
SEQ ID NO: 114
DNA - Lactococcus lactis
atg tat aca gta gga gat tac ctg tta gac cga tta cac gag ttg gga
Met Tyr Thr Val Gly Asp Tyr Leu Leu Asp Arg Leu His Glu Leu Gly
att gaa gaa att ttt gga gtt cct ggt gac tat aac tta caa ttt tta
Ile Glu Glu Ile Phe Gly Val Pro Gly Asp Tyr Asn Leu Gln Phe Leu
gat caa att att tca cgc gaa gat atg aaa tgg att gga aat gct aat
Asp Gln Ile Ile Ser Arg Glu Asp Met Lys Trp Ile Gly Asn Ala Asn
gaa tta aat gct tct tat atg gct gat ggt tat gct cgt act aaa aaa
Glu Leu Asn Ala Ser Tyr Met Ala Asp Gly Tyr Ala Arg Thr Lys Lys
gct gcc gca ttt ctc acc aca ttt gga gtc ggc gaa ttg agt gcg atc
Ala Ala Ala Phe Leu Thr Thr Phe Gly Val Gly Glu Leu Ser Ala Ile
aat gga ctg gca gga agt tat gcc gaa aat tta cca gta gta gaa att
Asn Gly Leu Ala Gly Ser Tyr Ala Glu Asn Leu Pro Val Val Glu Ile
gtt ggt tca cca act tca aaa gta caa aat gac gga aaa ttt gtc cat
Val Gly Ser Pro Thr Ser Lys Val Gln Asn Asp Gly Lys Phe Val His
cat aca cta gca gat ggt gat ttt aaa cac ttt atg aag atg cat gaa
His Thr Leu Ala Asp Gly Asp Phe Lys His Phe Met Lys Met His Glu
cct gtt aca gca gcg cgg act tta ctg aca gca gaa aat gcc aca tat
Pro Val Thr Ala Ala Arg Thr Leu Leu Thr Ala Glu Asn Ala Thr Tyr
gaa att gac cga gta ctt tct caa tta cta aaa gaa aga aaa cca gtc
Glu Ile Asp Arg Val Leu Ser Gln Leu Leu Lys Glu Arg Lys Pro Val
tat att aac tta cca gtc gat gtt gct gca gca aaa gca gag aag cct
Tyr Ile Asn Leu Pro Val Asp Val Ala Ala Ala Lys Ala Glu Lys Pro
gca tta tct tta gaa aaa gaa agc tct aca aca aat aca act gaa caa
Ala Leu Ser Leu Glu Lys Glu Ser Ser Thr Thr Asn Thr Thr Glu Gln
gtg att ttg agt aag att gaa gaa agt ttg aaa aat gcc caa aaa cca
Val Ile Leu Ser Lys Ile Glu Glu Ser Leu Lys Asn Ala Gln Lys Pro
gta gtg att gca gga cac gaa gta att agt ttt ggt tta gaa aaa acg
Val Val Ile Ala Gly His Glu Val Ile Ser Phe Gly Leu Glu Lys Thr
gta act cag ttt gtt tca gaa aca aaa cta ccg att acg aca cta aat
Val Thr Gln Phe Val Ser Glu Thr Lys Leu Pro Ile Thr Thr Leu Asn
ttt ggt aaa agt gct gtt gat gaa tct ttg ccc tca ttt tta gga ata
Phe Gly Lys Ser Ala Val Asp Glu Ser Leu Pro Ser Phe Leu Gly Ile
tat aac ggg aaa ctt tca gaa atc agt ctt aaa aat ttt gtg gag tcc
Tyr Asn Gly Lys Leu Ser Glu Ile Ser Leu Lys Asn Phe Val Glu Ser
gca gac ttt atc cta atg ctt gga gtg aag ctt acg gac tcc tca aca
Ala Asp Phe Ile Leu Met Leu Gly Val Lys Leu Thr Asp Ser Ser Thr
ggt gca ttc aca cat cat tta gat gaa aat aaa atg att tca cta aac
Gly Ala Phe Thr His His Leu Asp Glu Asn Lys Met Ile Ser Leu Asn
ata gat gaa gga ata att ttc aat aaa gtg gta gaa gat ttt gat ttt
Ile Asp Glu Gly Ile Ile Phe Asn Lys Val Val Glu Asp Phe Asp Phe
aga gca gtg gtt tct tct tta tca gaa tta aaa gga ata gaa tat gaa
Arg Ala Val Val Ser Ser Leu Ser Glu Leu Lys Gly Ile Glu Tyr Glu
gga caa tat att gat aag caa tat gaa gaa ttt att cca tca agt gct
Gly Gln Tyr Ile Asp Lys Gln Tyr Glu Glu Phe Ile Pro Ser Ser Ala
ccc tta tca caa gac cgt cta tgg cag gca gtt gaa agt ttg act caa
Pro Leu Ser Gln Asp Arg Leu Trp Gln Ala Val Glu Ser Leu Thr Gln
agc aat gaa aca atc gtt gct gaa caa gga acc tca ttt ttt gga gct
Ser Asn Glu Thr Ile Val Ala Glu Gln Gly Thr Ser Phe Phe Gly Ala
tca aca att ttc tta aaa tca aat agt cgt ttt att gga caa cct tta
Ser Thr Ile Phe Leu Lys Ser Asn Ser Arg Phe Ile Gly Gln Pro Leu
tgg ggt tct att gga tat act ttt cca gcg gct tta gga agc caa att
Trp Gly Ser Ile Gly Tyr Thr Phe Pro Ala Ala Leu Gly Ser Gln Ile
gcg gat aaa gag agc aga cac ctt tta ttt att ggt gat ggt tca ctt
Ala Asp Lys Glu Ser Arg His Leu Leu Phe Ile Gly Asp Gly Ser Leu
caa ctt acc gta caa gaa tta gga cta tca atc aga gaa aaa ctc aat
Gln Leu Thr Val Gln Glu Leu Gly Leu Ser Ile Arg Glu Lys Leu Asn
cca att tgt ttt atc ata aat aat gat ggt tat aca gtt gaa aga gaa
Pro Ile Cys Phe Ile Ile Asn Asn Asp Gly Tyr Thr Val Glu Arg Glu
atc cac gga cct act caa agt tat aac gac att cca atg tgg aat tac
Ile His Gly Pro Thr Gln Ser Tyr Asn Asp Ile Pro Met Trp Asn Tyr
tcg aaa tta cca gaa aca ttt gga gca aca gaa gat cgt gta gta tca
Ser Lys Leu Pro Glu Thr Phe Gly Ala Thr Glu Asp Arg Val Val Ser
aaa att gtt aga aca gag aat gaa ttt gtg tct gtc atg aaa gaa gcc
Lys Ile Val Arg Thr Glu Asn Glu Phe Val Ser Val Met Lys Glu Ala
caa gca gat gtc aat aga atg tat tgg ata gaa cta gtt ttg gaa aaa
Gln Ala Asp Val Asn Arg Met Tyr Trp Ile Glu Leu Val Leu Glu Lys
gaa gat gcg cca aaa tta ctg aaa aaa atg ggt aaa tta ttt gct gag
Glu Asp Ala Pro Lys Leu Leu Lys Lys Met Gly Lys Leu Phe Ala Glu
caa aat aaa tag
Gln Asn Lys
SEQ ID NO: 115
PRT - Lactococcus lactis
Met Tyr Thr Val Gly Asp Tyr Leu Leu Asp Arg Leu His Glu Leu Gly
Ile Glu Glu Ile Phe Gly Val Pro Gly Asp Tyr Asn Leu Gln Phe Leu
Asp Gln Ile Ile Ser Arg Glu Asp Met Lys Trp Ile Gly Asn Ala Asn
Glu Leu Asn Ala Ser Tyr Met Ala Asp Gly Tyr Ala Arg Thr Lys Lys
Ala Ala Ala Phe Leu Thr Thr Phe Gly Val Gly Glu Leu Ser Ala Ile
Asn Gly Leu Ala Gly Ser Tyr Ala Glu Asn Leu Pro Val Val Glu Ile
Val Gly Ser Pro Thr Ser Lys Val Gln Asn Asp Gly Lys Phe Val His
His Thr Leu Ala Asp Gly Asp Phe Lys His Phe Met Lys Met His Glu
Pro Val Thr Ala Ala Arg Thr Leu Leu Thr Ala Glu Asn Ala Thr Tyr
Glu Ile Asp Arg Val Leu Ser Gln Leu Leu Lys Glu Arg Lys Pro Val
Tyr Ile Asn Leu Pro Val Asp Val Ala Ala Ala Lys Ala Glu Lys Pro
Ala Leu Ser Leu Glu Lys Glu Ser Ser Thr Thr Asn Thr Thr Glu Gln
Val Ile Leu Ser Lys Ile Glu Glu Ser Leu Lys Asn Ala Gln Lys Pro
Val Val Ile Ala Gly His Glu Val Ile Ser Phe Gly Leu Glu Lys Thr
Val Thr Gln Phe Val Ser Glu Thr Lys Leu Pro Ile Thr Thr Leu Asn
Phe Gly Lys Ser Ala Val Asp Glu Ser Leu Pro Ser Phe Leu Gly Ile
Tyr Asn Gly Lys Leu Ser Glu Ile Ser Leu Lys Asn Phe Val Glu Ser
Ala Asp Phe Ile Leu Met Leu Gly Val Lys Leu Thr Asp Ser Ser Thr
Gly Ala Phe Thr His His Leu Asp Glu Asn Lys Met Ile Ser Leu Asn
Ile Asp Glu Gly Ile Ile Phe Asn Lys Val Val Glu Asp Phe Asp Phe
Arg Ala Val Val Ser Ser Leu Ser Glu Leu Lys Gly Ile Glu Tyr Glu
Gly Gln Tyr Ile Asp Lys Gln Tyr Glu Glu Phe Ile Pro Ser Ser Ala
Pro Leu Ser Gln Asp Arg Leu Trp Gln Ala Val Glu Ser Leu Thr Gln
Ser Asn Glu Thr Ile Val Ala Glu Gln Gly Thr Ser Phe Phe Gly Ala
Ser Thr Ile Phe Leu Lys Ser Asn Ser Arg Phe Ile Gly Gln Pro Leu
Trp Gly Ser Ile Gly Tyr Thr Phe Pro Ala Ala Leu Gly Ser Gln Ile
Ala Asp Lys Glu Ser Arg His Leu Leu Phe Ile Gly Asp Gly Ser Leu
Gln Leu Thr Val Gln Glu Leu Gly Leu Ser Ile Arg Glu Lys Leu Asn
Pro Ile Cys Phe Ile Ile Asn Asn Asp Gly Tyr Thr Val Glu Arg Glu
Ile His Gly Pro Thr Gln Ser Tyr Asn Asp Ile Pro Met Trp Asn Tyr
Ser Lys Leu Pro Glu Thr Phe Gly Ala Thr Glu Asp Arg Val Val Ser
Lys Ile Val Arg Thr Glu Asn Glu Phe Val Ser Val Met Lys Glu Ala
Gln Ala Asp Val Asn Arg Met Tyr Trp Ile Glu Leu Val Leu Glu Lys
Glu Asp Ala Pro Lys Leu Leu Lys Lys Met Gly Lys Leu Phe Ala Glu
Gln Asn Lys
SEQ ID NO: 116
DNA - Artificial
Lactococcus lactis branched chain alpha-ketoacid decarboxylase
KdcA codon optimised gene
atgtatactg ttggtgatta tctgctggac cgtctgcatg aactgggcat tgaagaaatc
ttcggtgtcc caggcgacta caacctgcag ttcctggacc agatcatctc ccgcgaagat
atgaaatgga tcggtaacgc aaacgagctg aacgcgtctt atatggctga tggttatgct
cgcaccaaaa aggctgcggc ctttctgacc acctttggtg tgggcgagct gagcgcgatc
aacggcctgg caggttccta cgctgagaac ctgccggtag tagaaatcgt tggttccccg
acctctaagg ttcagaacga cggcaaattc gtacatcaca ccctggcgga cggcgatttt
aagcacttta tgaaaatgca cgaaccggtc accgccgctc gcactctgct gaccgcggaa
aacgcaacgt acgagatcga tcgtgtactg tcccagctgc tgaaagaacg taaaccggtg
tatatcaatc tgccggttga tgtcgctgcg gccaaagcag agaaaccggc actgtccctg
gagaaggaga gctccactac taacaccacc gaacaggtta tcctgtccaa aattgaagaa
tctctgaaaa acgcacagaa accggtggtt atcgcaggtc acgaggttat ctccttcggc
ctggagaaaa ctgttactca attcgtctct gaaacgaaac tgccgatcac gaccctgaac
tttggcaagt ccgcagttga cgaatctctg ccttctttcc tgggcattta caacggcaaa
ctgtccgaga tctccctgaa gaacttcgta gaatccgctg actttatcct gatgctgggt
gtgaaactga ccgactcctc taccggtgcg ttcacgcacc atctggatga aaacaaaatg
atcagcctga acatcgacga gggtatcatc ttcaacaagg tagttgaaga tttcgacttc
cgtgctgttg tcagcagcct gtccgagctg aaaggcattg agtacgaggg tcaatacatc
gataaacagt acgaagagtt tattccgtct tctgcaccgc tgagccagga ccgcctgtgg
caggcagttg agtccctgac gcagtccaac gaaactatcg tagcggaaca aggtacctct
ttcttcggtg cttctaccat ctttctgaag tccaactctc gctttatcgg tcagccgctg
tggggttcta tcggttacac gttcccggct gcgctgggta gccagatcgc tgataaagag
tctcgtcatc tgctgttcat cggtgatggt tccctgcagc tgactgtaca ggaactgggt
ctgtctatcc gtgaaaaact gaacccgatt tgttttatca tcaataacga tggctacact
gttgagcgtg aaattcatgg tccgactcag tcttacaacg atattccgat gtggaactac
tctaaactgc cggaaacctt cggtgcaact gaggatcgcg tcgtgagcaa gattgtgcgt
actgagaacg agttcgtatc tgttatgaaa gaggcgcagg cagatgtgaa ccgcatgtac
tggatcgaac tggttctgga aaaagaggat gcaccgaaac tgctgaagaa aatgggtaaa
ctgtttgcgg agcagaacaa gtaa
SEQ ID NO: 117
DNA - Lactococcus lactis
atg tat aca gta gga gat tac cta tta gac cga tta cac gag tta gga
Met Tyr Thr Val Gly Asp Tyr Leu Leu Asp Arg Leu His Glu Leu Gly
att gaa gaa att ttt gga gtc cct gga gac tat aac tta caa ttt tta
Ile Glu Glu Ile Phe Gly Val Pro Gly Asp Tyr Asn Leu Gln Phe Leu
gat caa att att tcc cac aag gat atg aaa tgg gtc gga aat gct aat
Asp Gln Ile Ile Ser His Lys Asp Met Lys Trp Val Gly Asn Ala Asn
gaa tta aat gct tca tat atg gct gat ggc tat gct cgt act aaa aaa
Glu Leu Asn Ala Ser Tyr Met Ala Asp Gly Tyr Ala Arg Thr Lys Lys
gct gcc gca ttt ctt aca acc ttt gga gta ggt gaa ttg agt gca gtt
Ala Ala Ala Phe Leu Thr Thr Phe Gly Val Gly Glu Leu Ser Ala Val
aat gga tta gca gga agt tac gcc gaa aat tta cca gta gta gaa ata
Asn Gly Leu Ala Gly Ser Tyr Ala Glu Asn Leu Pro Val Val Glu Ile
gtg gga tca cct aca tca aaa gtt caa aat gaa gga aaa ttt gtt cat
Val Gly Ser Pro Thr Ser Lys Val Gln Asn Glu Gly Lys Phe Val His
cat acg ctg gct gac ggt gat ttt aaa cac ttt atg aaa atg cac gaa
His Thr Leu Ala Asp Gly Asp Phe Lys His Phe Met Lys Met His Glu
cct gtt aca gca gct cga act tta ctg aca gca gaa aat gca acc gtt
Pro Val Thr Ala Ala Arg Thr Leu Leu Thr Ala Glu Asn Ala Thr Val
gaa att gac cga gta ctt tct gca cta tta aaa gaa aga aaa cct gtc
Glu Ile Asp Arg Val Leu Ser Ala Leu Leu Lys Glu Arg Lys Pro Val
tat atc aac tta cca gtt gat gtt gct gct gca aaa gca gag aaa ccc
Tyr Ile Asn Leu Pro Val Asp Val Ala Ala Ala Lys Ala Glu Lys Pro
tca ctc cct ttg aaa aag gaa aac tca act tca aat aca agt gac caa
Ser Leu Pro Leu Lys Lys Glu Asn Ser Thr Ser Asn Thr Ser Asp Gln
gaa att ttg aac aaa att caa gaa agc ttg aaa aat gcc aaa aaa cca
Glu Ile Leu Asn Lys Ile Gln Glu Ser Leu Lys Asn Ala Lys Lys Pro
atc gtg att aca gga cat gaa ata att agt ttt ggc tta gaa aaa aca
Ile Val Ile Thr Gly His Glu Ile Ile Ser Phe Gly Leu Glu Lys Thr
gtc act caa ttt att tca aag aca aaa cta cct att acg aca tta aac
Val Thr Gln Phe Ile Ser Lys Thr Lys Leu Pro Ile Thr Thr Leu Asn
ttt ggt aaa agt tca gtt gat gaa gcc ctc cct tca ttt tta gga atc
Phe Gly Lys Ser Ser Val Asp Glu Ala Leu Pro Ser Phe Leu Gly Ile
tat aat ggt aca ctc tca gag cct aat ctt aaa gaa ttc gtg gaa tca
Tyr Asn Gly Thr Leu Ser Glu Pro Asn Leu Lys Glu Phe Val Glu Ser
gcc gac ttc atc ttg atg ctt gga gtt aaa ctc aca gac tct tca aca
Ala Asp Phe Ile Leu Met Leu Gly Val Lys Leu Thr Asp Ser Ser Thr
gga gcc ttc act cat cat tta aat gaa aat aaa atg att tca ctg aat
Gly Ala Phe Thr His His Leu Asn Glu Asn Lys Met Ile Ser Leu Asn
ata gat gaa gga aaa ata ttt aac gaa aga atc caa aat ttt gat ttt
Ile Asp Glu Gly Lys Ile Phe Asn Glu Arg Ile Gln Asn Phe Asp Phe
gaa tcc ctc atc tcc tct ctc tta gac cta agc gaa ata gaa tac aaa
Glu Ser Leu Ile Ser Ser Leu Leu Asp Leu Ser Glu Ile Glu Tyr Lys
gga aaa tat atc gat aaa aag caa gaa gac ttt gtt cca tca aat gcg
Gly Lys Tyr Ile Asp Lys Lys Gln Glu Asp Phe Val Pro Ser Asn Ala
ctt tta tca caa gac cgc cta tgg caa gca gtt gaa aac cta act caa
Leu Leu Ser Gln Asp Arg Leu Trp Gln Ala Val Glu Asn Leu Thr Gln
agc aat gaa aca atc gtt gct gaa caa ggg aca tca ttc ttt ggc gct
Ser Asn Glu Thr Ile Val Ala Glu Gln Gly Thr Ser Phe Phe Gly Ala
tca tca att ttc tta aaa tca aag agt cat ttt att ggt caa ccc tta
Ser Ser Ile Phe Leu Lys Ser Lys Ser His Phe Ile Gly Gln Pro Leu
tgg gga tca att gga tat aca ttc cca gca gca tta gga agc caa att
Trp Gly Ser Ile Gly Tyr Thr Phe Pro Ala Ala Leu Gly Ser Gln Ile
gca gat aaa gaa agc aga cac ctt tta ttt att ggt gat ggt tca ctt
Ala Asp Lys Glu Ser Arg His Leu Leu Phe Ile Gly Asp Gly Ser Leu
caa ctt aca gtg caa gaa tta gga tta gca atc aga gaa aaa att aat
Gln Leu Thr Val Gln Glu Leu Gly Leu Ala Ile Arg Glu Lys Ile Asn
cca att tgc ttt att atc aat aat gat ggt tat aca gtc gaa aga gaa
Pro Ile Cys Phe Ile Ile Asn Asn Asp Gly Tyr Thr Val Glu Arg Glu
att cat gga cca aat caa agc tac aat gat att cca atg tgg aat tac
Ile His Gly Pro Asn Gln Ser Tyr Asn Asp Ile Pro Met Trp Asn Tyr
tca aaa tta cca gaa tcg ttt gga gca aca gaa gat cga gta gtc tca
Ser Lys Leu Pro Glu Ser Phe Gly Ala Thr Glu Asp Arg Val Val Ser
aaa atc gtt aga act gaa aat gaa ttt gtg tct gtc atg aaa gaa gct
Lys Ile Val Arg Thr Glu Asn Glu Phe Val Ser Val Met Lys Glu Ala
caa gca gat cca aat aga atg tac tgg att gag tta att ttg gca aaa
Gln Ala Asp Pro Asn Arg Met Tyr Trp Ile Glu Leu Ile Leu Ala Lys
gaa ggt gca cca aaa gta ctg aaa aaa atg ggc aaa cta ttt gct gaa
Glu Gly Ala Pro Lys Val Leu Lys Lys Met Gly Lys Leu Phe Ala Glu
caa aat aaa tca taa
Gln Asn Lys Ser
SEQ ID NO: 118
PRT - Lactococcus lactis
Met Tyr Thr Val Gly Asp Tyr Leu Leu Asp Arg Leu His Glu Leu Gly
Ile Glu Glu Ile Phe Gly Val Pro Gly Asp Tyr Asn Leu Gln Phe Leu
Asp Gln Ile Ile Ser His Lys Asp Met Lys Trp Val Gly Asn Ala Asn
Glu Leu Asn Ala Ser Tyr Met Ala Asp Gly Tyr Ala Arg Thr Lys Lys
Ala Ala Ala Phe Leu Thr Thr Phe Gly Val Gly Glu Leu Ser Ala Val
Asn Gly Leu Ala Gly Ser Tyr Ala Glu Asn Leu Pro Val Val Glu Ile
Val Gly Ser Pro Thr Ser Lys Val Gln Asn Glu Gly Lys Phe Val His
His Thr Leu Ala Asp Gly Asp Phe Lys His Phe Met Lys Met His Glu
Pro Val Thr Ala Ala Arg Thr Leu Leu Thr Ala Glu Asn Ala Thr Val
Glu Ile Asp Arg Val Leu Ser Ala Leu Leu Lys Glu Arg Lys Pro Val
Tyr Ile Asn Leu Pro Val Asp Val Ala Ala Ala Lys Ala Glu Lys Pro
Ser Leu Pro Leu Lys Lys Glu Asn Ser Thr Ser Asn Thr Ser Asp Gln
Glu Ile Leu Asn Lys Ile Gln Glu Ser Leu Lys Asn Ala Lys Lys Pro
Ile Val Ile Thr Gly His Glu Ile Ile Ser Phe Gly Leu Glu Lys Thr
Val Thr Gln Phe Ile Ser Lys Thr Lys Leu Pro Ile Thr Thr Leu Asn
Phe Gly Lys Ser Ser Val Asp Glu Ala Leu Pro Ser Phe Leu Gly Ile
Tyr Asn Gly Thr Leu Ser Glu Pro Asn Leu Lys Glu Phe Val Glu Ser
Ala Asp Phe Ile Leu Met Leu Gly Val Lys Leu Thr Asp Ser Ser Thr
Gly Ala Phe Thr His His Leu Asn Glu Asn Lys Met Ile Ser Leu Asn
Ile Asp Glu Gly Lys Ile Phe Asn Glu Arg Ile Gln Asn Phe Asp Phe
Glu Ser Leu Ile Ser Ser Leu Leu Asp Leu Ser Glu Ile Glu Tyr Lys
Gly Lys Tyr Ile Asp Lys Lys Gln Glu Asp Phe Val Pro Ser Asn Ala
Leu Leu Ser Gln Asp Arg Leu Trp Gln Ala Val Glu Asn Leu Thr Gln
Ser Asn Glu Thr Ile Val Ala Glu Gln Gly Thr Ser Phe Phe Gly Ala
Ser Ser Ile Phe Leu Lys Ser Lys Ser His Phe Ile Gly Gln Pro Leu
Trp Gly Ser Ile Gly Tyr Thr Phe Pro Ala Ala Leu Gly Ser Gln Ile
Ala Asp Lys Glu Ser Arg His Leu Leu Phe Ile Gly Asp Gly Ser Leu
Gln Leu Thr Val Gln Glu Leu Gly Leu Ala Ile Arg Glu Lys Ile Asn
Pro Ile Cys Phe Ile Ile Asn Asn Asp Gly Tyr Thr Val Glu Arg Glu
Ile His Gly Pro Asn Gln Ser Tyr Asn Asp Ile Pro Met Trp Asn Tyr
Ser Lys Leu Pro Glu Ser Phe Gly Ala Thr Glu Asp Arg Val Val Ser
Lys Ile Val Arg Thr Glu Asn Glu Phe Val Ser Val Met Lys Glu Ala
Gln Ala Asp Pro Asn Arg Met Tyr Trp Ile Glu Leu Ile Leu Ala Lys
Glu Gly Ala Pro Lys Val Leu Lys Lys Met Gly Lys Leu Phe Ala Glu
Gln Asn Lys Ser
SEQ ID NO: 119
DNA - Artificial
Lactococcus lactis-ketoisovalerate decarboxylase KivD codon
optimised gene
atgtatactg ttggtgatta cctgctggat cgtctgcatg aactgggcat cgaggaaatt
ttcggcgtac ctggtgacta taacctgcag ttcctggatc agatcatttc ccacaaagat
atgaaatggg ttggtaacgc gaacgagctg aatgcaagct acatggctga cggttatgca
cgcaccaaga aagctgcggc gttcctgact acttttggcg tcggcgagct gtctgcggta
aacggtctgg ccggctccta cgcggaaaac ctgccggtag tagaaatcgt cggttccccg
acctctaaag ttcagaacga gggtaaattc gtgcaccata ctctggccga tggtgacttc
aaacacttca tgaagatgca cgaaccggtc actgctgctc gtacgctgct gaccgcggaa
aatgcgactg tcgagattga tcgtgtactg agcgcactgc tgaaagaacg caagcctgta
tacatcaacc tgccggttga tgtcgcggcc gccaaagcgg aaaaaccatc tctgccgctg
aaaaaggaga acagcacctc taacaccagc gaccaggaaa tcctgaacaa gatccaggag
tctctgaaga acgctaaaaa gccgatcgta atcaccggcc atgagattat ctctttcggt
ctggagaaaa ctgtcaccca gttcatcagc aaaaccaaac tgccgatcac caccctgaac
ttcggtaaat cctccgttga cgaagcgctg ccgtcctttc tgggtattta caacggcact
ctgtctgagc cgaacctgaa agagttcgtg gagtctgcgg attttatcct gatgctgggc
gtgaaactga cggattcctc caccggtgca ttcacccacc acctgaatga gaataaaatg
atctctctga acattgatga gggcaaaatc ttcaacgagc gtattcagaa cttcgatttc
gaatccctga tctcctccct gctggatctg tccgagattg aatataaagg caaatacatt
gataagaagc aagaggactt cgtaccgtct aacgcgctgc tgagccagga ccgtctgtgg
caagctgtgg aaaacctgac ccagtccaac gaaaccatcg tggcggaaca gggtacctcc
ttcttcggtg ctagctctat cttcctgaaa tctaaaagcc acttcatcgg tcagccactg
tggggctcta ttggctacac cttcccggca gcgctgggtt cccaaatcgc agacaaagaa
tcccgccacc tgctgttcat tggtgacggc tctctgcaac tgaccgtaca ggagctgggt
ctggcgattc gtgagaaaat caacccgatt tgtttcatca tcaacaacga tggctacact
gttgagcgtg agatccacgg cccgaaccag tcctacaacg acattccgat gtggaactac
tctaaactgc cggaatcctt cggtgcgact gaagaccgtg tcgtaagcaa gatcgtccgt
accgaaaacg aattcgtgtc tgtcatgaaa gaagcacagg cggacccgaa ccgcatgtac
tggatcgagc tgattctggc taaagagggc gcgccaaaag tactgaaaaa gatgggtaaa
ctgttcgcag aacagaacaa atcctaa
SEQ ID NO: 120
DNA - Mycobacterium tuberculosis
gtg gcc aac ata agt tca cca ttc ggg caa aac gaa tgg ctg gtc gaa
Val Ala Asn Ile Ser Ser Pro Phe Gly Gln Asn Glu Trp Leu Val Glu
gag atg tac cgc aag ttc cgc gac gac ccc tcc tcg gtc gat ccc agc
Glu Met Tyr Arg Lys Phe Arg Asp Asp Pro Ser Ser Val Asp Pro Ser
tgg cac gag ttc ctg gtt gac tac agc ccc gaa ccc acc tcc caa cca
Trp His Glu Phe Leu Val Asp Tyr Ser Pro Glu Pro Thr Ser Gln Pro
gct gcc gaa cca acc cgg gtt acc tcg cca ctc gtt gcc gag cgg gcc
Ala Ala Glu Pro Thr Arg Val Thr Ser Pro Leu Val Ala Glu Arg Ala
gct gcg gcc gcc ccg cag gca ccc ccc aag ccg gcc gac acc gcg gcc
Ala Ala Ala Ala Pro Gln Ala Pro Pro Lys Pro Ala Asp Thr Ala Ala
gcg ggc aac ggc gtg gtc gcc gca ctg gcc gcc aaa act gcc gtt ccc
Ala Gly Asn Gly Val Val Ala Ala Leu Ala Ala Lys Thr Ala Val Pro
ccg cca gcc gaa ggt gac gag gta gcg gtg ctg cgc ggc gcc gcc gcg
Pro Pro Ala Glu Gly Asp Glu Val Ala Val Leu Arg Gly Ala Ala Ala
gcc gtc gtc aag aac atg tcc gcg tcg ttg gag gtg ccg acg gcg acc
Ala Val Val Lys Asn Met Ser Ala Ser Leu Glu Val Pro Thr Ala Thr
agc gtc cgg gcg gtc ccg gcc aag cta ctg atc gac aac cgg atc gtc
Ser Val Arg Ala Val Pro Ala Lys Leu Leu Ile Asp Asn Arg Ile Val
atc aac aac cag ttg aag cgg acc cgc ggc ggc aag atc tcg ttc acg
Ile Asn Asn Gln Leu Lys Arg Thr Arg Gly Gly Lys Ile Ser Phe Thr
cat ttg ctg ggc tac gcc ctg gtg cag gcg gtg aag aaa ttc ccg aac
His Leu Leu Gly Tyr Ala Leu Val Gln Ala Val Lys Lys Phe Pro Asn
atg aac cgg cac tac acc gaa gtc gac ggc aag ccc acc gcg gtc acg
Met Asn Arg His Tyr Thr Glu Val Asp Gly Lys Pro Thr Ala Val Thr
ccg gcg cac acc aat ctc ggc ctg gcg atc gac ctg caa ggc aag gac
Pro Ala His Thr Asn Leu Gly Leu Ala Ile Asp Leu Gln Gly Lys Asp
ggg aag cgt tcc ctg gtg gtg gcc ggc atc aag cgg tgc gag acc atg
Gly Lys Arg Ser Leu Val Val Ala Gly Ile Lys Arg Cys Glu Thr Met
cga ttc gcg cag ttc gtc acg gcc tac gaa gac atc gta cgc cgg gcc
Arg Phe Ala Gln Phe Val Thr Ala Tyr Glu Asp Ile Val Arg Arg Ala
cgc gac ggc aag ctg acc act gaa gac ttt gcc ggc gtg acg att tcg
Arg Asp Gly Lys Leu Thr Thr Glu Asp Phe Ala Gly Val Thr Ile Ser
ctg acc aat ccc gga acc atc ggc acc gtg cat tcg gtg ccg cgg ctg
Leu Thr Asn Pro Gly Thr Ile Gly Thr Val His Ser Val Pro Arg Leu
atg ccc ggc cag ggc gcc atc atc ggc gtg ggc gcc atg gaa tac ccc
Met Pro Gly Gln Gly Ala Ile Ile Gly Val Gly Ala Met Glu Tyr Pro
gcc gag ttt caa ggc gcc agc gag gaa cgc atc gcc gag ctg ggc atc
Ala Glu Phe Gln Gly Ala Ser Glu Glu Arg Ile Ala Glu Leu Gly Ile
ggc aaa ttg atc act ttg acc tcc acc tac gac cac cgc atc atc cag
Gly Lys Leu Ile Thr Leu Thr Ser Thr Tyr Asp His Arg Ile Ile Gln
ggc gcg gaa tcg ggc gac ttc ctg cgc acc atc cac gag ttg ctg ctc
Gly Ala Glu Ser Gly Asp Phe Leu Arg Thr Ile His Glu Leu Leu Leu
tcg gat ggc ttc tgg gac gag gtc ttc cgc gaa ctg agc atc cca tat
Ser Asp Gly Phe Trp Asp Glu Val Phe Arg Glu Leu Ser Ile Pro Tyr
ctg ccg gtg cgc tgg agc acc gac aac ccc gac tcg atc gtc gac aag
Leu Pro Val Arg Trp Ser Thr Asp Asn Pro Asp Ser Ile Val Asp Lys
aac gct cgc gtc atg aac ttg atc gcg gcc tac cgc aac cgc ggc cat
Asn Ala Arg Val Met Asn Leu Ile Ala Ala Tyr Arg Asn Arg Gly His
ctg atg gcc gat acc gac ccg ctg cgg ttg gac aaa gct cgg ttc cgc
Leu Met Ala Asp Thr Asp Pro Leu Arg Leu Asp Lys Ala Arg Phe Arg
agt cac ccc gac ctc gaa gtg ctg acc cac ggc ctg acg ctg tgg gat
Ser His Pro Asp Leu Glu Val Leu Thr His Gly Leu Thr Leu Trp Asp
ctc gat cgg gtg ttc aag gtc gac ggc ttt gcc ggt gcg cag tac aag
Leu Asp Arg Val Phe Lys Val Asp Gly Phe Ala Gly Ala Gln Tyr Lys
aaa ctg cgc gac gtg ctg ggc ttg ctg cgc gat gcc tac tgc cgc cac
Lys Leu Arg Asp Val Leu Gly Leu Leu Arg Asp Ala Tyr Cys Arg His
atc ggc gtg gag tac gcc cat atc ctc gac ccc gaa caa aag gag tgg
Ile Gly Val Glu Tyr Ala His Ile Leu Asp Pro Glu Gln Lys Glu Trp
ctc gaa caa cgg gtc gag acc aag cac gtc aaa ccc act gtg gcc caa
Leu Glu Gln Arg Val Glu Thr Lys His Val Lys Pro Thr Val Ala Gln
cag aaa tac atc ctc agc aag ctc aac gcc gcc gag gcc ttt gaa acg
Gln Lys Tyr Ile Leu Ser Lys Leu Asn Ala Ala Glu Ala Phe Glu Thr
ttc cta cag acc aag tac gtc ggc cag aag cgg ttc tcg ctg gaa ggc
Phe Leu Gln Thr Lys Tyr Val Gly Gln Lys Arg Phe Ser Leu Glu Gly
gcc gaa agc gtg atc ccg atg atg gac gcg gcg atc gac cag tgc gct
Ala Glu Ser Val Ile Pro Met Met Asp Ala Ala Ile Asp Gln Cys Ala
gag cac ggc ctc gac gag gtg gtc atc ggg atg ccg cac cgg ggc cgg
Glu His Gly Leu Asp Glu Val Val Ile Gly Met Pro His Arg Gly Arg
ctc aac gtg ctg gcc aac atc gtc ggc aag ccg tac tcg cag atc ttc
Leu Asn Val Leu Ala Asn Ile Val Gly Lys Pro Tyr Ser Gln Ile Phe
acc gag ttc gag ggc aac ctg aat ccg tcg cag gcg cac ggc tcc ggt
Thr Glu Phe Glu Gly Asn Leu Asn Pro Ser Gln Ala His Gly Ser Gly
gac gtc aag tac cac ctg ggc gcc acc ggg ctg tac ctg cag atg ttc
Asp Val Lys Tyr His Leu Gly Ala Thr Gly Leu Tyr Leu Gln Met Phe
ggc gac aac gac att cag gtg tcg ctg acc gcc aac ccg tcg cat ctg
Gly Asp Asn Asp Ile Gln Val Ser Leu Thr Ala Asn Pro Ser His Leu
gag gcc gtc gac ccg gtg ctg gag gga ttg gtg cgg gcc aag cag gat
Glu Ala Val Asp Pro Val Leu Glu Gly Leu Val Arg Ala Lys Gln Asp
ctg ctc gac cac gga agc atc gac agc gac ggc caa cgg gcg ttc tcg
Leu Leu Asp His Gly Ser Ile Asp Ser Asp Gly Gln Arg Ala Phe Ser
gtg gtg ccg ctg atg ttg cat ggc gat gcc gcg ttc gcc ggt cag ggt
Val Val Pro Leu Met Leu His Gly Asp Ala Ala Phe Ala Gly Gln Gly
gtg gtc gcc gag acg ctg aac ctg gcg aat ctg ccg ggc tac cgc gtc
Val Val Ala Glu Thr Leu Asn Leu Ala Asn Leu Pro Gly Tyr Arg Val
ggc ggc acc atc cac atc atc gtc aac aac cag atc ggc ttc acc acc
Gly Gly Thr Ile His Ile Ile Val Asn Asn Gln Ile Gly Phe Thr Thr
gcg ccc gag tat tcc agg tcc agc gag tac tgc acc gac gtc gca aag
Ala Pro Glu Tyr Ser Arg Ser Ser Glu Tyr Cys Thr Asp Val Ala Lys
atg atc ggg gca ccg atc ttt cac gtc aac ggc gac gac ccg gag gcg
Met Ile Gly Ala Pro Ile Phe His Val Asn Gly Asp Asp Pro Glu Ala
tgt gtc tgg gtg gcg cgg ttg gcg gtg gac ttc cga caa cgg ttc aag
Cys Val Trp Val Ala Arg Leu Ala Val Asp Phe Arg Gln Arg Phe Lys
aag gac gtc gtc atc gac atg ctg tgc tac cgc cgc cgc ggg cac aac
Lys Asp Val Val Ile Asp Met Leu Cys Tyr Arg Arg Arg Gly His Asn
gag ggt gac gac ccg tcg atg acc aac ccc tac gtg tac gac gtc gtc
Glu Gly Asp Asp Pro Ser Met Thr Asn Pro Tyr Val Tyr Asp Val Val
gac acc aag cgc ggg gcc cgc aaa agc tac acc gaa gcc ctg atc gga
Asp Thr Lys Arg Gly Ala Arg Lys Ser Tyr Thr Glu Ala Leu Ile Gly
cgt ggc gac atc tcg atg aag gag gcc gag gac gcg ctg cgc gac tac
Arg Gly Asp Ile Ser Met Lys Glu Ala Glu Asp Ala Leu Arg Asp Tyr
cag ggc cag ctg gaa cgg gtg ttc aac gaa gtg cgc gag ctg gag aag
Gln Gly Gln Leu Glu Arg Val Phe Asn Glu Val Arg Glu Leu Glu Lys
cac ggt gtg cag ccg agc gag tcg gtc gag tcc gac cag atg att ccc
His Gly Val Gln Pro Ser Glu Ser Val Glu Ser Asp Gln Met Ile Pro
gcg ggg ctg gcc act gcg gtg gac aag tcg ctg ctg gcc cgg atc ggc
Ala Gly Leu Ala Thr Ala Val Asp Lys Ser Leu Leu Ala Arg Ile Gly
gat gcg ttc ctc gcc ttg ccg aac ggc ttc acc gcg cac ccg cga gtc
Asp Ala Phe Leu Ala Leu Pro Asn Gly Phe Thr Ala His Pro Arg Val
caa ccg gtg ctg gag aag cgc cgg gag atg gcc tat gaa ggc aag atc
Gln Pro Val Leu Glu Lys Arg Arg Glu Met Ala Tyr Glu Gly Lys Ile
gac tgg gcc ttt ggc gag ctg ctg gcg ctg ggc tcg ctg gtg gcc gaa
Asp Trp Ala Phe Gly Glu Leu Leu Ala Leu Gly Ser Leu Val Ala Glu
ggc aag ctg gtg cgc ttg tcg ggg cag gac agc cgc cgc ggc acc ttc
Gly Lys Leu Val Arg Leu Ser Gly Gln Asp Ser Arg Arg Gly Thr Phe
tcc cag cgg cat tcg gtt ctc atc gac cgc cac act ggc gag gag ttc
Ser Gln Arg His Ser Val Leu Ile Asp Arg His Thr Gly Glu Glu Phe
aca cca ctg cag ctg ctg gcg acc aac tcc gac ggc agc ccg acc ggc
Thr Pro Leu Gln Leu Leu Ala Thr Asn Ser Asp Gly Ser Pro Thr Gly
gga aag ttc ctg gtc tac gac tcg cca ctg tcg gag tac gcc gcc gtc
Gly Lys Phe Leu Val Tyr Asp Ser Pro Leu Ser Glu Tyr Ala Ala Val
ggc ttc gag tac ggc tac act gtg ggc aat ccg gac gcc gtg gtg ctc
Gly Phe Glu Tyr Gly Tyr Thr Val Gly Asn Pro Asp Ala Val Val Leu
tgg gag gcg cag ttc ggc gac ttc gtc aac ggc gcg cag tcg atc atc
Trp Glu Ala Gln Phe Gly Asp Phe Val Asn Gly Ala Gln Ser Ile Ile
gac gag ttc atc agc tcc ggt gag gcc aag tgg ggc caa ttg tcc aac
Asp Glu Phe Ile Ser Ser Gly Glu Ala Lys Trp Gly Gln Leu Ser Asn
gtc gtg ctg ctg tta ccg cac ggg cac gag ggg cag gga ccc gac
Val Val Leu Leu Leu Pro His Gly His Glu Gly Gln Gly Pro Asp
cac act tct gcc cgg atc gaa cgc ttc ttg cag ttg tgg gcg gaa
His Thr Ser Ala Arg Ile Glu Arg Phe Leu Gln Leu Trp Ala Glu
ggt tcg atg acc atc gcg atg ccg tcg act ccg tcg aac tac ttc
Gly Ser Met Thr Ile Ala Met Pro Ser Thr Pro Ser Asn Tyr Phe
cac ctg cta cgc cgg cat gcc ctg gac ggc atc caa cgc ccg ctg
His Leu Leu Arg Arg His Ala Leu Asp Gly Ile Gln Arg Pro Leu
atc gtg ttc acg ccc aag tcg atg ttg cgt cac aag gcc gcc gtc
Ile Val Phe Thr Pro Lys Ser Met Leu Arg His Lys Ala Ala Val
agc gaa atc aag gac ttc acc gag atc aag ttc cgc tca gtg ctg
Ser Glu Ile Lys Asp Phe Thr Glu Ile Lys Phe Arg Ser Val Leu
gag gaa ccc acc tat gag gac ggc atc gga gac cgc aac aag gtc
Glu Glu Pro Thr Tyr Glu Asp Gly Ile Gly Asp Arg Asn Lys Val
agc cgg atc ctg ctg acc agt ggc aag ctg tat tac gag ctg gcc
Ser Arg Ile Leu Leu Thr Ser Gly Lys Leu Tyr Tyr Glu Leu Ala
gcc cgc aag gcc aag gac aac cgc aat gac ctc gcg atc gtg cgg
Ala Arg Lys Ala Lys Asp Asn Arg Asn Asp Leu Ala Ile Val Arg
ctt gaa cag ctc gcc ccg ctg ccc agg cgt cga ctg cgt gaa acg
Leu Glu Gln Leu Ala Pro Leu Pro Arg Arg Arg Leu Arg Glu Thr
ctg gac cgc tac gag aac gtc aag gag ttc ttc tgg gtc caa gag
Leu Asp Arg Tyr Glu Asn Val Lys Glu Phe Phe Trp Val Gln Glu
gaa ccg gcc aac cag ggt gcg tgg ccg cga ttc ggg ctc gaa cta
Glu Pro Ala Asn Gln Gly Ala Trp Pro Arg Phe Gly Leu Glu Leu
ccc gag ctg ctg cct gac aag ttg gcc ggg atc aag cga atc tcg
Pro Glu Leu Leu Pro Asp Lys Leu Ala Gly Ile Lys Arg Ile Ser
cgc cgg gcg atg tca gcc ccg tcg tca ggc tcg tcg aag gtg cac
Arg Arg Ala Met Ser Ala Pro Ser Ser Gly Ser Ser Lys Val His
gcc gtc gaa cag cag gag atc ctc gac gag gcg ttc ggc tga
Ala Val Glu Gln Gln Glu Ile Leu Asp Glu Ala Phe Gly
SEQ ID NO: 121
PRT - Mycobacterium tuberculosis
Val Ala Asn Ile Ser Ser Pro Phe Gly Gln Asn Glu Trp Leu Val Glu
Glu Met Tyr Arg Lys Phe Arg Asp Asp Pro Ser Ser Val Asp Pro Ser
Trp His Glu Phe Leu Val Asp Tyr Ser Pro Glu Pro Thr Ser Gln Pro
Ala Ala Glu Pro Thr Arg Val Thr Ser Pro Leu Val Ala Glu Arg Ala
Ala Ala Ala Ala Pro Gln Ala Pro Pro Lys Pro Ala Asp Thr Ala Ala
Ala Gly Asn Gly Val Val Ala Ala Leu Ala Ala Lys Thr Ala Val Pro
Pro Pro Ala Glu Gly Asp Glu Val Ala Val Leu Arg Gly Ala Ala Ala
Ala Val Val Lys Asn Met Ser Ala Ser Leu Glu Val Pro Thr Ala Thr
Ser Val Arg Ala Val Pro Ala Lys Leu Leu Ile Asp Asn Arg Ile Val
Ile Asn Asn Gln Leu Lys Arg Thr Arg Gly Gly Lys Ile Ser Phe Thr
His Leu Leu Gly Tyr Ala Leu Val Gln Ala Val Lys Lys Phe Pro Asn
Met Asn Arg His Tyr Thr Glu Val Asp Gly Lys Pro Thr Ala Val Thr
Pro Ala His Thr Asn Leu Gly Leu Ala Ile Asp Leu Gln Gly Lys Asp
Gly Lys Arg Ser Leu Val Val Ala Gly Ile Lys Arg Cys Glu Thr Met
Arg Phe Ala Gln Phe Val Thr Ala Tyr Glu Asp Ile Val Arg Arg Ala
Arg Asp Gly Lys Leu Thr Thr Glu Asp Phe Ala Gly Val Thr Ile Ser
Leu Thr Asn Pro Gly Thr Ile Gly Thr Val His Ser Val Pro Arg Leu
Met Pro Gly Gln Gly Ala Ile Ile Gly Val Gly Ala Met Glu Tyr Pro
Ala Glu Phe Gln Gly Ala Ser Glu Glu Arg Ile Ala Glu Leu Gly Ile
Gly Lys Leu Ile Thr Leu Thr Ser Thr Tyr Asp His Arg Ile Ile Gln
Gly Ala Glu Ser Gly Asp Phe Leu Arg Thr Ile His Glu Leu Leu Leu
Ser Asp Gly Phe Trp Asp Glu Val Phe Arg Glu Leu Ser Ile Pro Tyr
Leu Pro Val Arg Trp Ser Thr Asp Asn Pro Asp Ser Ile Val Asp Lys
Asn Ala Arg Val Met Asn Leu Ile Ala Ala Tyr Arg Asn Arg Gly His
Leu Met Ala Asp Thr Asp Pro Leu Arg Leu Asp Lys Ala Arg Phe Arg
Ser His Pro Asp Leu Glu Val Leu Thr His Gly Leu Thr Leu Trp Asp
Leu Asp Arg Val Phe Lys Val Asp Gly Phe Ala Gly Ala Gln Tyr Lys
Lys Leu Arg Asp Val Leu Gly Leu Leu Arg Asp Ala Tyr Cys Arg His
Ile Gly Val Glu Tyr Ala His Ile Leu Asp Pro Glu Gln Lys Glu Trp
Leu Glu Gln Arg Val Glu Thr Lys His Val Lys Pro Thr Val Ala Gln
Gln Lys Tyr Ile Leu Ser Lys Leu Asn Ala Ala Glu Ala Phe Glu Thr
Phe Leu Gln Thr Lys Tyr Val Gly Gln Lys Arg Phe Ser Leu Glu Gly
Ala Glu Ser Val Ile Pro Met Met Asp Ala Ala Ile Asp Gln Cys Ala
Glu His Gly Leu Asp Glu Val Val Ile Gly Met Pro His Arg Gly Arg
Leu Asn Val Leu Ala Asn Ile Val Gly Lys Pro Tyr Ser Gln Ile Phe
Thr Glu Phe Glu Gly Asn Leu Asn Pro Ser Gln Ala His Gly Ser Gly
Asp Val Lys Tyr His Leu Gly Ala Thr Gly Leu Tyr Leu Gln Met Phe
Gly Asp Asn Asp Ile Gln Val Ser Leu Thr Ala Asn Pro Ser His Leu
Glu Ala Val Asp Pro Val Leu Glu Gly Leu Val Arg Ala Lys Gln Asp
Leu Leu Asp His Gly Ser Ile Asp Ser Asp Gly Gln Arg Ala Phe Ser
Val Val Pro Leu Met Leu His Gly Asp Ala Ala Phe Ala Gly Gln Gly
Val Val Ala Glu Thr Leu Asn Leu Ala Asn Leu Pro Gly Tyr Arg Val
Gly Gly Thr Ile His Ile Ile Val Asn Asn Gln Ile Gly Phe Thr Thr
Ala Pro Glu Tyr Ser Arg Ser Ser Glu Tyr Cys Thr Asp Val Ala Lys
Met Ile Gly Ala Pro Ile Phe His Val Asn Gly Asp Asp Pro Glu Ala
Cys Val Trp Val Ala Arg Leu Ala Val Asp Phe Arg Gln Arg Phe Lys
Lys Asp Val Val Ile Asp Met Leu Cys Tyr Arg Arg Arg Gly His Asn
Glu Gly Asp Asp Pro Ser Met Thr Asn Pro Tyr Val Tyr Asp Val Val
Asp Thr Lys Arg Gly Ala Arg Lys Ser Tyr Thr Glu Ala Leu Ile Gly
Arg Gly Asp Ile Ser Met Lys Glu Ala Glu Asp Ala Leu Arg Asp Tyr
Gln Gly Gln Leu Glu Arg Val Phe Asn Glu Val Arg Glu Leu Glu Lys
His Gly Val Gln Pro Ser Glu Ser Val Glu Ser Asp Gln Met Ile Pro
Ala Gly Leu Ala Thr Ala Val Asp Lys Ser Leu Leu Ala Arg Ile Gly
Asp Ala Phe Leu Ala Leu Pro Asn Gly Phe Thr Ala His Pro Arg Val
Gln Pro Val Leu Glu Lys Arg Arg Glu Met Ala Tyr Glu Gly Lys Ile
Asp Trp Ala Phe Gly Glu Leu Leu Ala Leu Gly Ser Leu Val Ala Glu
Gly Lys Leu Val Arg Leu Ser Gly Gln Asp Ser Arg Arg Gly Thr Phe
Ser Gln Arg His Ser Val Leu Ile Asp Arg His Thr Gly Glu Glu Phe
Thr Pro Leu Gln Leu Leu Ala Thr Asn Ser Asp Gly Ser Pro Thr Gly
Gly Lys Phe Leu Val Tyr Asp Ser Pro Leu Ser Glu Tyr Ala Ala Val
Gly Phe Glu Tyr Gly Tyr Thr Val Gly Asn Pro Asp Ala Val Val Leu
Trp Glu Ala Gln Phe Gly Asp Phe Val Asn Gly Ala Gln Ser Ile Ile
Asp Glu Phe Ile Ser Ser Gly Glu Ala Lys Trp Gly Gln Leu Ser Asn
Val Val Leu Leu Leu Pro His Gly His Glu Gly Gln Gly Pro Asp
His Thr Ser Ala Arg Ile Glu Arg Phe Leu Gln Leu Trp Ala Glu
Gly Ser Met Thr Ile Ala Met Pro Ser Thr Pro Ser Asn Tyr Phe
His Leu Leu Arg Arg His Ala Leu Asp Gly Ile Gln Arg Pro Leu
Ile Val Phe Thr Pro Lys Ser Met Leu Arg His Lys Ala Ala Val
Ser Glu Ile Lys Asp Phe Thr Glu Ile Lys Phe Arg Ser Val Leu
Glu Glu Pro Thr Tyr Glu Asp Gly Ile Gly Asp Arg Asn Lys Val
Ser Arg Ile Leu Leu Thr Ser Gly Lys Leu Tyr Tyr Glu Leu Ala
Ala Arg Lys Ala Lys Asp Asn Arg Asn Asp Leu Ala Ile Val Arg
Leu Glu Gln Leu Ala Pro Leu Pro Arg Arg Arg Leu Arg Glu Thr
Leu Asp Arg Tyr Glu Asn Val Lys Glu Phe Phe Trp Val Gln Glu
Glu Pro Ala Asn Gln Gly Ala Trp Pro Arg Phe Gly Leu Glu Leu
Pro Glu Leu Leu Pro Asp Lys Leu Ala Gly Ile Lys Arg Ile Ser
Arg Arg Ala Met Ser Ala Pro Ser Ser Gly Ser Ser Lys Val His
Ala Val Glu Gln Gln Glu Ile Leu Asp Glu Ala Phe Gly
SEQ ID NO: 122
DNA - Artificial
Mycobacterium tuberculosis-ketoglutarate decarboxylase Kgd
codon optimised gene
atggctaata tctcctctcc gtttggtcag aatgaatggc tggtagaaga aatgtaccgt
aaattccgcg atgacccgtc ctctgtggac ccgtcctggc atgaattcct ggtagactac
agcccggagc cgaccagcca accggcagcg gaaccaaccc gcgttacttc tccgctggta
gcggaacgtg cagctgctgc cgcgcctcag gcgccgccta aaccggcgga tactgccgca
gccggtaacg gtgtggtggc cgcactggct gctaagactg cggttccgcc gccagcagaa
ggcgatgaag ttgcagtcct gcgcggtgcg gcggctgcag tggtgaaaaa catgagcgcg
tccctggagg taccgaccgc cacgagcgtg cgcgcggtcc ctgctaaact gctgattgat
aaccgtattg tgatcaacaa ccagctgaaa cgtacccgtg gtggcaagat ctccttcact
catctgctgg gttatgcact ggtacaagcg gttaagaaat tccctaacat gaaccgtcat
tacactgagg tcgacggtaa accgacggct gttactccgg cacacacgaa cctgggcctg
gcgatcgacc tgcaaggtaa agatggtaag cgctccctgg tagttgcggg tattaaacgt
tgcgaaacca tgcgtttcgc acaattcgta accgcctacg aggacattgt ccgccgtgct
cgtgatggca aactgaccac cgaagatttt gcgggcgtta ctattagcct gaccaaccca
ggcaccatcg gcaccgtgca cagcgtacct cgtctgatgc cgggccaagg tgcgattatc
ggtgtgggtg ccatggagta cccggcagaa tttcagggtg cttctgaaga gcgcatcgcc
gagctgggta ttggtaaact gatcaccctg acttctacct atgaccaccg catcattcag
ggcgcagaat ccggtgactt cctgcgcact attcacgaac tgctgctgtc cgacggtttc
tgggatgaag tttttcgtga actgagcatc ccatatctgc cagttcgctg gtccaccgac
aatccggact ctatcgttga caaaaacgct cgcgtaatga acctgatcgc tgcttatcgt
aatcgtggtc acctgatggc tgatacggat ccgctgcgcc tggataaagc tcgtttccgt
tcccacccgg acctggaagt gctgacccat ggtctgactc tgtgggatct ggaccgcgtg
ttcaaagtag atggtttcgc gggtgctcag tacaagaagc tgcgtgacgt gctgggtctg
ctgcgtgatg cgtactgtcg tcacattggt gtggagtacg cccacattct ggatccggaa
cagaaagaat ggctggagca gcgtgtcgag accaaacacg taaaaccgac cgtagcgcag
cagaaatata tcctgtccaa actgaacgcc gccgaggctt tcgaaacttt cctgcagacc
aagtacgtgg gccagaaacg cttcagcctg gagggtgcgg aaagcgttat tccgatgatg
gatgcagcta tcgatcagtg cgcggaacat ggtctggatg aagtcgttat cggtatgccg
caccgtggtc gcctgaacgt actggcaaac atcgtcggta aaccatattc tcagatcttc
acggaattcg agggcaacct gaacccgtcc caagcccacg gctccggcga cgtaaaatat
catctgggtg ctaccggcct gtatctgcag atgttcggtg ataacgacat ccaggtatct
ctgactgcta acccgagcca cctggaggcg gttgatcctg ttctggaagg tctggttcgc
gccaaacagg atctgctgga ccacggctct atcgacagcg atggccagcg tgcattcagc
gttgtaccgc tgatgctgca tggcgacgcg gcgttcgccg gtcagggtgt cgtagcagaa
actctgaacc tggcgaacct gcctggctat cgcgtgggtg gcaccattca catcatcgtt
aacaaccaaa tcggtttcac cacggcaccg gagtatagcc gttctagcga atattgcacc
gacgtagcca aaatgatcgg tgcgccgatc ttccatgtaa acggtgacga tccagaggcc
tgcgtgtggg tggctcgtct ggccgtagac ttccgccagc gttttaagaa agatgtggtt
atcgacatgc tgtgctaccg ccgtcgtggt cacaacgaag gtgatgatcc gtctatgact
aacccgtatg tctatgacgt ggtggacacc aagcgtggtg cacgcaaatc ttacacggag
gccctgatcg gtcgtggcga catctctatg aaagaagcgg aagacgctct gcgtgattac
cagggtcagc tggaacgtgt gttcaatgag gtgcgtgagc tggaaaagca cggcgtacaa
ccgtccgaat ccgtagagtc cgatcagatg atccctgctg gtctggcaac tgctgttgat
aaaagcctgc tggcgcgtat cggcgacgca ttcctggcgc tgccgaatgg ctttaccgcg
cacccgcgcg tacagccggt actggaaaaa cgtcgtgaaa tggcctacga aggtaaaatc
gattgggcct tcggtgagct gctggccctg ggctctctgg tggctgaggg caagctggta
cgcctgagcg gccaggactc ccgtcgcggc actttttctc agcgtcacag cgtcctgatc
gatcgtcaca ccggcgaaga attcacgccg ctgcaactgc tggctactaa ctccgatggt
agcccgaccg gtggtaagtt cctggtgtac gattccccgc tgtccgaata tgctgcagtt
ggtttcgagt atggttacac cgttggcaac ccggacgcag tggttctgtg ggaagcgcag
ttcggcgatt tcgttaacgg tgcccagtcc attatcgatg agtttattag cagcggcgag
gccaaatggg gccagctgtc taacgttgtg ctgctgctgc ctcacggcca cgagggtcaa
ggcccggacc acacctccgc ccgtatcgaa cgcttcctgc agctgtgggc tgaaggctct
atgaccatcg cgatgccgtc taccccaagc aactacttcc acctgctgcg tcgccacgca
ctggacggca ttcagcgccc gctgatcgtt ttcaccccaa aatccatgct gcgccacaaa
gcagctgttt ctgaaatcaa agattttacg gaaattaaat tccgttctgt gctggaagaa
ccaacctacg aagacggtat tggcgaccgc aacaaggtaa gccgtatcct gctgacctcc
ggcaaactgt actacgagct ggcagcacgt aaggcaaaag ataaccgcaa cgacctggcc
atcgtccgcc tggaacagct ggcgccactg ccacgccgtc gcctgcgtga aaccctggat
cgctacgaaa acgtaaaaga attcttctgg gtgcaggaag aaccggcaaa ccagggtgcg
tggccgcgct ttggtctgga actgccggaa ctgctgccgg ataaactggc aggtatcaag
cgcatcagcc gtcgcgctat gagcgccccg tcttctggta gctctaaagt acacgctgta
gaacagcaag agatcctgga tgaggccttc ggctaa
SEQ ID NO: 123
DNA - Artificial sequence
Forward primer for amplification of Bacillus subtilis
aminotransferase x
ggggacaagt ttgtacaaaa aagcaggcta ggaggaatta accatgaagg ttttagtcaa
tggccggctg attg
SEQ ID NO: 124
DNA - Artificial
Reverse primer for amplification of Bacillus subtilis
aminotransferase x
ggggaccact ttgtacaaga aagctgggtt tatgaaatgc tagcagcctg ttgaatgctt
tc
SEQ ID NO: 125
DNA - Artificial
Forward primer for amplification of Bacillus subtilis
aminotransferase y
ggggacaagt ttgtacaaaa aagcaggcta ggaggaatta accatgactc atgatttgat
agaaaaaagt aaaaagcacc tc
SEQ ID NO: 126
DNA - Artificial
Reverse primer for amplification of Bacillus subtilis
aminotransferase y
ggggaccact ttgtacaaga aagctgggtt caatcttcaa ggctcgtaac ctcgtgg
SEQ ID NO: 127
DNA - Artificial
Forward primer for amplification of Rhodobacter sphaeroides
aminotransferase
ggggacaagt ttgtacaaaa aagcaggcta ggaggaatta accatgcccg gttgcggggg
cttg
SEQ ID NO: 128
DNA - Artificial
Reverse primer for amplification of Rhodobacter sphaeroides
aminotransferase
ggggaccact ttgtacaaga aagctgggtt cagacggcgg ccggttcttt c
SEQ ID NO: 129
DNA - Artificial
Forward primer for amplification of Legionella pneumophila
aminotransferase
ggggacaagt ttgtacaaaa aagcaggcta ggaggaatta accatgagta tcgcatttgt
taacggcaag tattgttg
SEQ ID NO: 130
DNA - Artificial
Reverse primer for amplification of Legionella pneumophila
aminotransferase
ggggaccact ttgtacaaga aagctgggtt tagtttacta gttgttggta ggaatcatta
attatcc
SEQ ID NO: 131
DNA - Artificial
Forward primer for amplification of Nitrosomonas europaea
aminotransferase
ggggacaagt ttgtacaaaa aagcaggcta ggaggaatta accatgattt acctcaatgg
caaatttctg ccgatg
SEQ ID NO: 132
DNA - Artificial
Reverse primer for amplification of Nitrosomonas europaea
aminotransferase
ggggaccact ttgtacaaga aagctgggtt tactggcgtg gagcatgccc
SEQ ID NO: 133
DNA - Artificial
Forward primer for amplification of Neisseria gonorrhoeae
aminotransferase
ggggacaagt ttgtacaaaa aagcaggcta ggaggaatta accatgagga taaatatgaa
ccgtaacgaa attttattc
SEQ ID NO: 134
DNA - Artificial
Reverse primer for amplification of Neisseria gonorrhoeae
aminotransferase
ggggaccact ttgtacaaga aagctgggtt catgcagcca tcgccttgaa cacttc
SEQ ID NO: 135
DNA - Artificial
Forward primer for amplification of Pseudomonas aeruginosa
aminotransferase
ggggacaagt ttgtacaaaa aagcaggcta ggaggaatta accatgtcga tggccgatcg
tgatgg
SEQ ID NO: 136
DNA - Artificial
Reverse primer for amplification of Pseudomonas aeruginosa
aminotransferase
SEQ ID NO: 136
ggggaccact ttgtacaaga aagctgggtt tacttgacca gggtacgcca ctc
SEQ ID NO: 137
DNA - Artificial
Forward primer for amplification of Rhodopseudomonas palustris
aminotransferase
ggggacaagt ttgtacaaaa aagcaggcta ggaggaatta accatgaagc tgataccgtg
ccgcgcc
SEQ ID NO: 138
DNA - Artificial
Reverse primer for amplification of Rhodopseudomonas palustris
aminotransferase
ggggaccact ttgtacaaga aagctgggtt caggcgaccg cgcggatcac c
SEQ ID NO: 139
DNA - Artificial
Forward primer for amplification of Bacillus subtilis
aminotransferase (gi16077991)
ggggacaagt ttgtacaaaa aagcaggcta ggaggaatta accatggaga tgatggggat
ggaaaacatt c
SEQ ID NO: 140
DNA - Artificial
Reverse primer for amplification of Bacillus subtilis
aminotransferase (gi16077991)
ggggaccact ttgtacaaga aagctgggtt tatatcgttt gaaagctttc tttcaccgtt
ttcac
SEQ ID NO: 141
DNA - Artificial
Forward primer for amplification of Pseudomonas aeruginosa
aminotransferase (gi9951072)
ggggacaagt ttgtacaaaa aagcaggcta ggaggaatta accatgaacg caagactgca
cgccac
SEQ ID NO: 142
DNA - Artificial
Reverse primer for amplification of Pseudomonas aeruginosa
aminotransferase (gi9951072)
ggggaccact ttgtacaaga aagctgggtt taccggtgac cggcgcgg
SEQ ID NO: 143
DNA - Artificial
Forward primer for amplification of Pseudomonas aeruginosa
aminotransferase (gi9951630)
ggggacaagt ttgtacaaaa aagcaggcta ggaggaatta accatgacaa tgaatgacga
gccgcagtc
SEQ ID NO: 144
DNA - Artificial
Reverse primer for amplification of Pseudomonas aeruginosa
aminotransferase (gi9951630)
ggggaccact ttgtacaaga aagctgggtt cagacgctgg cgcggatgg
SEQ ID NO: 145
DNA - Methanococcus jannaschii
atgacaaaag tgctggtgat gtttatggat ttcttatttg agaacagctg gaaagcagtt
tgtccctaca atccaaagtt ggatttaaag gacatttata tttatgacac aaccctaaga
gatggagagc aaaccccagg agtttgcttt accaaagaac aaaaattgga gattgcaagg
aagttggatg aacttggatt aaagcagatt gaagctggct tcccaatagt atctgaaaga
gaagcagata tagttaaaac aattgctaat gaagggctaa atgctgatat cttagcttta
tgcagggctt taaagaaaga tatagataaa gcaatagagt gcgatgtaga tgggattatt
accttcatag caacatctcc tctccactta aaatataaat tcaacaacaa aagcttagat
gaaatattag agatgggagt tgaggcagtt gagtatgcaa aggaacatgg cttatttgtt
gctttctctg cagaggatgc gacaagaaca ccaatagagg acttgattaa agtgcataaa
gccgctgaag aggctggagc agatagggtt catatagcag acacaactgg ctgtgctacc
ccccaaagta tggagtttat atgtaaaaca ttgaaggaga acttaaaaaa ggcacatatt
ggagtgcatt gtcacaacga ctttggattt gcagttataa attcaatata tggtttaatt
ggaggagcta aggcagtttc aacaacagtt aatggtattg gagagagggc agggaatgca
gctttagaag agctaattat ggctttaact gtcttgtatg atgttgattt gggattaaac
ttggaggttc ttccagagtt atgcagaatg gttgaggaat actctggaat aaagatgcca

aagaacaaac caatagttgg agagcttgta tttgctcatg aaagtggaat tcacgttgat
gctgtcatag agaatccatt aacctatgaa cccttccttc cagagaaaat agggcttaag
agaaatattt tgttagggaa gcattctgga tgcagagccg ttgcctataa gctaaaactt
atgggaattg attacgatag agagatgttg tgcgagattg ttaaaaaggt taaagagatt
agagaggaag gtaaatttat aactgatgaa gtctttaagg agattgttga agaagtttta
aggaagagaa ataaaaatta a
SEQ ID NO: 146
DNA - Methanococcus jannaschii
atgattatta agggaagagc tcacaaattt ggggatgatg tagatacaga cgcaataatt
ccaggacctt acttaaggac tacagaccct tacgagttag cttcacactg catggcaggg
atagatgaaa acttcccgaa aaaggttaag gagggggatg tgatagttgc tggagagaat
tttggttgtg gttcaagtag ggagcaggct gtaatagcaa taaaatactg tggtattaag
gctgtgatag caaaaagctt tgcaagaata ttctatagaa atgcaataaa cgttggatta
ataccaataa tagcaaatac agatgaaatt aaagacggag acatagtaga gattgattta
gataaagaag agattgtaat aaccaataaa aacaaaacaa taaagtgtga aacaccaaaa
ggtttagaaa gagaaatatt ggctgctggt ggcttagtca attatttaaa aaagagaaaa
ctaatacaat caaaaaaagg tgtaaaaaca tga
SEQ ID NO: 47
DNA - Methanococcus jannaschii
ttgacattgg tagagaagat actatcaaaa aaagttggtt atgaagtttg tgcaggagat
agcatagagg ttgaagttga tttggcaatg acacacgatg gaacaacacc tttagcatac
aaagctttaa aggaaatgag tgatagtgtt tggaatccag ataaaatagt cgttgccttt
gaccacaatg ttccaccaaa cacagttaaa gctgctgaaa tgcaaaaatt agctttggag
tttgttaaaa gatttggcat taaaaatttc cataaaggtg gagaaggcat ctgtcatcaa
atcttagctg aaaattatgt tttgccaaac atgtttgtag ctggtggaga cagccataca
tgcacacatg gagcttttgg agcttttgct actggctttg gagctactga tatggcttac
atctatgcaa caggagaaac atggattaaa gtgccaaaaa caattagggt agatatagtt
ggaaaaaatg aaaatgtttc tgccaaagat attgttttaa gggtttgtaa ggaaattggg
agaagaggag caacatacat ggctattgag tatggtggag aggttgttaa aaacatggac
atggatggaa ggctaacttt atgcaacatg gcaatagaga tgggaggaaa aacaggagtg
atagaggctg atgaaattac ttatgattat ttaaagaaag agagaggact ttctgatgag
gatatagcta aattaaaaaa agagagaata acagtaaata gagatgaagc aaactactat
aaggagatag aaattgacat aacagatatg gaagaacaag ttgctgttcc acaccaccca
gataacgtaa agccaattag tgatgttgaa gggactgaga taaatcaagt ttttattggg
agttgcacaa atggaaggtt gagtgattta agagaagcag ctaaatattt aaaaggtagg
gaggttcata aagatgttaa gctaattgtt atcccggcat caaaaaaggt atttttgcaa
gcgttaaaag agggtattat agatatcttt gttaaagctg gggcgatgat ttgcactccg
ggatgcggac cttgcttagg agctcatcaa ggggttttgg ctgagggaga aatttgttta
tcaacaacaa acagaaactt taaaggaagg atggggcata taaatagcta tatttacttg
gcatctccaa agattgccgc aataagtgca gttaagggat atataaccaa caaattggat
taa
SEQ ID NO: 148
DNA - Methanococcus jannaschii
atgatgaagg tgtgtgttat agaaggggat ggaataggaa aagaagtgat tccagaggcc
ataaaaatat taaatgagtt gggagagttt gaaataataa aaggagaggc aggattagaa
tgtttaaaaa aatatggtaa tgcacttcca gaggatacaa tagaaaaagc taaagaggca
gatattattt tgtttggggc tataacctca ccaaagccag gggaagttca aaattataaa
agccctataa taacgttgag gaagatgttt catttatatg caaatgtaag accaataaac
aactttggaa ttggacaatt aattgggaaa attgcagatt atgaattctt aaatgctaag
aatattgata tagttattat aagagagaat acggaagatt tatatgttgg tagagagaga
ttagaaaatg atacagcaat agctgagagg gttataacaa gaaagggtag cgagagaata
ataagatttg catttgaata tgctataaaa aataatagga aaaaggtatc ttgcatccat
aaagctaatg ttttaagaat aactgatggt ttattcttag aggtttttaa tgaaataaaa
aaacattata atatagaggc agatgattat ttagttgatt caacagctat gaacttaata
aaacatcctg aaaaatttga tgttattgtt acaacaaaca tgtttgggga tattttatca
gatgaggcat ctgcattaat tggaggactt ggtttagctc cttcagcaaa tataggagat
gataaagcat tatttgagcc agttcatggt tcagctccag atatagctgg gaaaggtata
gcaaatccaa tggcatctat attaagtatt gctatgcttt ttgattatat tggagagaaa
gaaaagggag atttgattag agaggcagtg aaatactgct taataaacaa aaaagttact
cctgacttgg gaggggattt aaagacaaaa gatgttggag acgaaattct aaattacatt
agaaagaagt taaagggata ttga
SEQ ID NO: 149
DNA - A. vinelandii homocitrate synthase
atggctagcg tgatcatcga cgacactacc ctgcgtgacg gtgaacagag tgccggggtc
gccttcaatg ccgacgagaa gatcgctatc gcccgcgcgc tcgccgaact gggcgtgccg
gagttggaga tcggcattcc cagcatgggc gaggaagagc gcgaggtgat gcacgccatc
gccggtctcg gcctgtcgtc tcgcctgctg gcctggtgcc ggctatgcga cgtcgatctc
gcggcggcgc gctccaccgg ggtgaccatg gtcgaccttt cgctgccggt ctccgacctg
atgctgcacc acaagctcaa tcgcgatcgc gactgggcct tgcgcgaagt ggccaggctg
gtcggcgaag cgcgcatggc cgggctcgag gtgtgcctgg gctgcgagga cgcctcgcgg
gcggatctgg agttcgtcgt gcaggtgggc gaagtggcgc aggccgccgg cgcccgtcgg
ctgcgcttcg ccgacaccgt cggggtcatg gagcccttcg gcatgctcga ccgcttccgt
ttcctcagcc ggcgcctgga catggagctg gaagtgcacg cccacgatga tttcgggctg
gccacggcca acaccctggc cgcggtgatg ggcggggcga ctcatatcaa caccacggtc
aacgggctcg gcgagcgtgc cggcaacgcc gcgctggaag agtgcgtgct ggcgctcaag
aacctccacg gtatcgacac cggtatcgat acccgcggca tcccggccat ctccgcgctg
gtcgagcggg cctcggggcg ccaggtggcc tggcagaaga gcgtggtcgg cgccggggtg
ttcactcacg aggccggtat ccacgtcgac ggactgctca agcatcggcg caactacgag
gggctgaatc ccgacgaact cggtcgcagc cacagtctgg tgctgggcaa gcattccggg
gcgcacatgg tgcgcaacac gtaccgcgat ctgggtatcg agctggcgga ctggcagagc
caagcgctgc tcggccgcat ccgtgccttc tccaccagga ccaagcgcag cccgcagcct
gccgagctgc aggatttcta tcggcagttg tgcgagcaag gcaatcccga actggccgca
ggaggaatgg catga
SEQ ID NO: 150
DNA - Artificial
Avine-WT-R-BamHI
aaattggatc ctcatgccat tcctcctgcg
SEQ ID NO: 151
DNA - Artificial
Avine-WT-F-SacI
aaattgagct ctttctccat acccgttttt ttgggctaac aggaggaatt aaccatggct
agcgtgatca tcgac
SEQ ID NO: 152
DNA - Artificial
Avine-WT-R-HindIII
aaattaaagc tttcatgcca ttcctcctgc g
SEQ ID NO: 153
DNA - Artificial
Avine-WT-F-HindIII
aaattaaagc tttttctcca tacccgtttt tttgggctaa caggaggaat taaccatggc
tagcgtgatc atcgac
SEQ ID NO: 154
DNA - Artificial
AksA-Avine-F
atggctagcg tgatcatcga c
SEQ ID NO: 155
DNA - Artificial
AksA-Avine-R1
aaattggcgc gcctcatgcc attcctcctg cg
SEQ ID NO: 156
DNA - Artificial
Pgal2-F2
aaattgttaa ctccagaagg cacatctatt ac
SEQ ID NO: 157
DNA - Artificial
Pgal2-R
cgtcgatgat cacgctagcc attatgaaag cctccttttt tttattatg
SEQ ID NO: 158
DNA - Artificial
mtSP
atggcctcca ctcgtgtcct cgcctctcgc ctggcctccc agatggctgc ttccgccaag
gttgcccgcc ctgctgtccg cgttgctcag gtcagcaagc gcaccatcca gactggctcc
cccctccaga ccctcaagcg cacccagatg acctccatcg tcaacgccac cacccgccag
gctttccaga agcgcgccta ctcttcc
SEQ ID NO: 159
DNA - Artificial
pF113-F-NsiI
aaattatgca tacagcatgg cctgcaacg
SEQ ID NO: 160
DNA - Artificial
pF113-R-AgeI
aaattaccgg tcagggttat tgtctcatga g
SEQ ID NO: 161
DNA - Artificial
AT-Vfl_for_Ec
aaatttggta ccgctaggag gaattaacca tg
SEQ ID NO: 162
DNA - Artificial
Kdc_for_Ec
aaatttacta gtggctagga ggaattacat atg
SEQ ID NO: 163
DNA - Artificial
Kdc_rev_Ec
aaatttaagc ttattacttg ttctgctccg caaac
SEQ ID NO: 164
DNA - Artificial
AT-Vfl-F
aaatttacta gtaagaattt ttgaggaggc aatataaatg aataaaccac agtcttg
SEQ ID NO: 165
DNA - Artificial
AT-Vfl-R
aaatttggat cctacaagaa agctgggttt ac
SEQ ID NO: 166
DNA - Artificial
AT-Vfl_rev_Ec
aaatttacta gtaagctggg tttacgcgac ttc
SEQ ID NO: 167
DNA - AksA_E. coli
atgaccaaag ttctggtaat gttcatggac ttcctgttcg aaaactcctg gaaagcggtt
tgcccgtaca acccgaaact ggatctgaaa gacatctaca tctacgacac cactctgcgt
gacggtgaac agactccggg cgtttgcttc accaaagagc agaagctgga aatcgctcgt
aagctggacg aactgggtct gaagcagatc gaagctggct tcccgatcgt ttctgaacgt
gaagctgaca tcgttaaaac tatcgctaac gaaggtctga acgctgacat cctggcactg
tgccgtgcgc tgaagaaaga catcgacaaa gcaatcgaat gcgacgttga cggtatcatc
actttcatcg caacttctcc gctgcacctg aaatacaaat tcaacaacaa atctctggat
gaaatcctgg aaatgggcgt tgaagcggta gaatacgcta aagagcacgg tctgttcgtt
gcattctctg cagaagatgc aactcgtact ccgatcgaag atctgatcaa agttcacaaa
gcagctgaag aagcgggtgc tgaccgcgtt cacatcgctg acaccactgg ctgcgcaact
ccgcagtcta tggaattcat ctgcaaaact ctgaaagaaa acctgaagaa agcacacatc
ggcgtacact gccacaacga cttcggtttc gctgttatca actccatcta cggtctgatc
ggtggtgcga aagcggtatc tactaccgtt aacggtatcg gtgaacgtgc tggtaacgct
gcactggaag agctgatcat ggcgctgacc gtactgtacg acgttgacct gggtctgaac
ctggaagttc tgccggaact gtgccgtatg gttgaagaat actccggtat caagatgccg
aaaaacaagc caatcgttgg tgaactggta ttcgctcacg aatccggtat ccacgttgac
gctgttatcg aaaacccgct gacttacgaa ccgttcctgc cggaaaaaat cggtctgaaa
cgtaacatcc tgctgggtaa gcactctggt tgccgtgctg ttgcttacaa gctgaaactg
atgggtatcg actacgaccg tgaaatgctg tgcgaaatcg ttaagaaagt taaagaaatc
cgtgaagaag gtaaattcat cactgacgaa gttttcaaag agatcgttga agaagttctg
cgtaagcgta acaaaaacta a
SEQ ID NO: 168
DNA - AksF_E. coli
atgatgaaag tttgcgttat cgaaggtgac ggtatcggta aagaagttat cccggaagct
atcaagatcc tgaacgaact gggtgaattc gaaatcatca aaggtgaagc gggtctggaa
tgcctgaaga aatacggtaa cgcactgcca gaagatacca tcgaaaaagc gaaagaagct
gacatcatcc tgttcggtgc aatcacttct ccgaagccgg gtgaagttca gaactacaaa
tctccgatca tcactctgcg taagatgttc cacctgtacg ctaacgtacg tccgatcaac
aacttcggta tcggtcagct gatcggtaag atcgctgact acgagttcct gaacgctaaa
aacatcgaca tcgttatcat ccgtgaaaac actgaagatc tgtacgttgg tcgtgaacgt
ctggaaaacg acactgctat cgctgagcgc gttatcactc gtaaaggttc tgaacgtatc
atccgcttcg cattcgaata cgcaatcaaa aacaaccgta agaaagtttc ctgcatccac
aaagctaacg tactgcgtat cactgacggt ctgttcctgg aagtattcaa cgaaatcaag
aaacactaca acatcgaagc tgacgactac ctggttgact ccactgcaat gaacctgatc
aagcacccgg aaaaattcga cgttatcgtt accactaaca tgttcggtga catcctgtct
gacgaagcgt ctgcactgat cggtggtctg ggtctggcac cgtctgctaa catcggtgac
gacaaagcgc tgttcgaacc ggttcacggt tctgcaccgg atatcgctgg taaaggtatc
gctaacccga tggcttctat cctgtctatc gcgatgctgt tcgactacat cggtgaaaaa
gagaaaggcg acctgatccg tgaagcggta aaatactgcc tgatcaacaa gaaagttact
ccggatctgg gtggtgacct gaaaaccaaa gacgttggtg acgaaatcct gaactacatc
cgtaagaaac tgaaaggtta ctaa
SEQ ID NO: 169
DNA - AksD_E. coli
atgactctgg ttgagaagat cctctccaag aaagttggtt acgaagtttg cgcaggcgac
tccatcgaag ttgaagttga cctggcgatg actcacgacg gtactactcc gctggcttac
aaagcgctga aagagatgtc tgactccgta tggaacccgg acaagatcgt tgttgcattc
gaccacaacg taccgccgaa caccgttaaa gcagctgaaa tgcagaagct ggcgctggaa
ttcgttaagc gcttcggtat caaaaacttc cacaaaggtg gtgaaggtat ctgccaccag
atcctggctg aaaactacgt tctgccgaac atgttcgttg ctggcggcga ctctcacacc
tgtactcacg gtgcattcgg tgcattcgca actggcttcg gtgcaactga catggcttac
atctacgcaa ctggcgaaac ctggatcaaa gttccgaaaa ctatccgcgt tgatatcgtt
ggtaaaaacg aaaacgtatc tgcgaaagac atcgttctgc gcgtttgcaa agaaatcggt
cgtcgcggtg caacttacat ggctatcgaa tacggtggtg aagttgttaa aaacatggac
atggacggtc gtctgactct gtgcaacatg gctatcgaaa tgggtggtaa aactggcgtt
atcgaagctg acgaaatcac ttacgactac ctgaagaaag agcgtggtct gtctgacgaa
gatatcgcta aactgaagaa agagcgtatc accgttaacc gtgacgaagc taactactac
aaagaaatcg aaatcgacat cactgacatg gaagaacagg ttgctgtacc gcaccacccg
gataacgtta agccaatctc tgacgttgaa ggtactgaaa tcaaccaggt attcatcggt
tcctgcacca acggtcgtct gtctgatctg cgtgaagctg cgaaatacct gaaaggtcgt
gaagttcaca aagacgttaa gctgatcgtt atcccggctt ccaagaaagt attcctgcag
gcgctgaaag aaggtatcat cgacatcttc gttaaagcgg gtgcgatgat ctgtactccg
ggttgcggtc cgtgcctggg tgcacaccag ggcgtactgg cagaaggtga aatctgcctg
tctactacca accgtaactt caaaggtcgt atgggtcaca tcaactctta catctacctg
gcttctccga aaatcgctgc tatctctgct gttaaaggtt acatcactaa caagctggat
taa
SEQ ID NO: 170
DNA - AksE_E. coli
atgatcatca aaggtcgtgc gcacaagttc ggtgacgacg ttgacactga cgctatcatc
ccaggtccgt acctccgtac tactgacccg tacgaactgg catctcactg catggcgggt
atcgacgaaa acttcccgaa gaaagttaaa gaaggtgacg ttatcgttgc tggcgaaaac
ttcggttgcg gttcttcccg tgagcaggct gttatcgcta tcaaatactg cggtatcaaa
gcggttatcg ctaaatcttt cgcacgtatc ttctaccgta acgcaatcaa cgtaggtctg
atcccgatca tcgctaacac cgacgaaatc aaagacggtg acatcgttga aatcgacctg
gataaagaag aaatcgttat cactaacaaa aacaaaacta tcaagtgcga aactccgaaa
ggtctggaac gtgaaatcct ggcagctggc ggtctggtta actacctgaa gaaacgtaag
ctgattcagt ccaagaaagg cgtaaaaact taa
SEQ ID NO: 171
DNA - AksA_S. cerevisiae
atgaccaagg ttttggtcat gttcatggac ttcttgtttg aaaactcctg gaaggccgtt
tgtccataca acccaaagtt ggacttgaag gacatctaca tctacgacac cactttaaga
gatggtgaac aaaccccagg tgtttgtttc accaaggaac aaaaattgga aattgccaga
aagttggacg aattgggttt gaaacaaatc gaagctggtt tcccaatcgt ttctgaaaga
gaagctgaca ttgtcaagac cattgccaac gaaggtttga acgctgatat cttagctcta
tgtagagctt tgaagaagga cattgacaag gccatcgaat gtgatgtcga tggtatcatc
actttcattg ctacttctcc attacatttg aaatacaagt tcaacaacaa atctttggac
gaaatcttgg aaatgggtgt tgaagctgtc gaatacgcca aggaacacgg tttattcgtt
gctttctctg ctgaagatgc taccagaact ccaattgaag atttgatcaa ggtccacaag
gctgctgaag aagctggtgc tgaccgtgtc cacattgctg acaccactgg ttgtgccact
ccacaatcca tggaatttat ctgtaagact ttgaaggaaa acttgaagaa ggctcacatt
ggtgttcact gtcacaacga tttcggtttc gctgtcatca actccatcta cggtttgatt
ggtggtgcca aggccgtttc caccaccgtc aacggtatcg gtgaaagagc tggtaacgct
gctttggaag aattgatcat ggctttgact gtcttatacg atgtcgattt gggtttgaac
ttggaagttt tgccagaatt gtgtagaatg gttgaagaat actctggtat caagatgcca
aagaacaagc caattgtcgg tgaattggtt ttcgctcatg aatctggtat tcacgttgac
gctgtcattg aaaacccatt gacctacgaa cctttcttgc cagaaaagat cggtttgaag
agaaacatcc tattaggtaa gcactctggt tgtcgtgctg ttgcttacaa attgaaattg
atgggtattg actacgacag agaaatgttg tgtgaaattg tcaagaaggt caaggaaatc
agagaagaag gtaagttcat cactgacgaa gttttcaagg aaatcgttga agaagttttg
agaaagagaa acaaaaatta a
SEQ ID NO: 172
DNA - AksD_S. cerevisiae
atgactttag tcgaaaagat cttatccaag aaggtcggtt acgaagtttg tgccggtgac
tctattgaag ttgaagttga cttggccatg acccacgacg gtactacccc attggcttac
aaggctttga aggaaatgtc tgactccgtc tggaacccag acaagattgt tgttgctttc
gaccacaacg ttccaccaaa caccgtcaag gctgctgaaa tgcaaaaatt ggctttggaa
tttgtcaaga gattcggtat caagaacttc cacaagggtg gtgaaggtat ctgtcaccaa
atcttggctg aaaactacgt tttgccaaac atgttcgttg ctggtggtga ctcccacact
tgtacccacg gtgctttcgg tgcctttgct accggtttcg gtgctactga catggcttac
atctacgcta ccggtgaaac ctggatcaag gttccaaaga ctatcagagt tgacattgtc
ggtaagaacg aaaacgtttc tgccaaggat atcgtcttga gagtttgtaa ggaaattggt
agaagaggtg ctacttacat ggccattgaa tacggtggtg aagttgtcaa gaacatggac
atggacggta gattgacttt gtgtaacatg gccattgaaa tgggtggtaa gactggtgtc
attgaagctg atgaaatcac ctacgactac ttgaagaagg aaagaggtct atccgatgaa
gatatcgcca aattgaagaa ggaaagaatc actgttaaca gagatgaagc taactactac
aaggaaattg aaattgatat cactgacatg gaagaacaag ttgctgttcc tcatcaccca
gacaatgtca agccaatttc tgacgtcgaa ggtactgaaa tcaaccaagt tttcatcggt
tcttgtacca acggtagatt atctgattta cgtgaagctg ctaagtactt gaaaggtcgt
gaagttcaca aggatgtcaa attgattgtc attccagctt ccaagaaggt tttcttgcaa
gctttgaagg aaggtatcat cgatatcttc gtcaaggctg gtgccatgat ctgtacccca
ggttgtggtc catgtttggg tgctcatcaa ggtgtcttgg ctgaaggtga aatctgtttg
tccaccacca acagaaactt caagggtaga atgggtcaca tcaactctta catctacttg
gcttctccaa agattgctgc catttctgct gtcaagggtt acatcactaa caaattggat
taa
SEQ ID NO: 173
DNA - AksE_S. cerevisiae
atgatcatca agggtcgtgc tcacaagttc ggtgacgatg ttgacactga tgctatcatt
ccaggtccat acttgagaac cactgaccca tacgaattgg cttctcactg tatggctggt
attgacgaaa acttcccaaa gaaggtcaag gaaggtgatg tcattgttgc tggtgaaaac
tttggttgtg gttcttccag agaacaagct gttattgcca tcaaatactg tggtatcaag
gctgtcattg ccaagtcttt cgctagaatc ttctacagaa acgccatcaa cgttggtttg
attccaatca ttgctaacac tgacgaaatc aaggatggtg acattgttga aatcgatttg
gacaaggaag aaattgttat caccaacaag aacaagacca tcaagtgtga aactccaaag
ggtttggaaa gagaaatctt ggctgctggt ggtttagtca actacttgaa gaagagaaag
ttgatccaat ccaagaaggg tgtcaaaacc taa
SEQ ID NO: 174
DNA - AksF_S. cerevisiae
atgatgaagg tttgtgtcat tgaaggtgac ggtattggta aggaagtcat tccagaagct
atcaagatct tgaatgaatt gggtgaattt gaaatcatca agggtgaagc tggtttggaa
tgtttgaaga aatacggtaa cgctttgcca gaagatacca ttgaaaaggc caaggaagct
gatatcatct tattcggtgc catcacttct ccaaagccag gtgaagttca aaactacaaa
tctccaatca tcactttgag aaagatgttc cacttgtacg ctaacgtcag accaatcaac
aacttcggta ttggtcaatt gattggtaag attgctgact acgaattttt gaatgccaag
aacattgaca ttgtcatcat cagagaaaac actgaagatt tgtacgttgg tcgtgaaaga
ttagaaaacg acactgccat tgctgaacgt gttatcacca gaaagggttc tgaaagaatc
atcagattcg ctttcgaata cgccatcaag aacaacagaa agaaggtttc ctgtatccac
aaggctaacg ttttgagaat caccgatggt ttattcttgg aagttttcaa cgaaatcaag
aagcactaca acattgaagc tgatgactac ttggttgact ccactgctat gaacttgatc
aagcatccag aaaagttcga tgtcattgtc accaccaaca tgttcggtga catcttatct
gacgaagctt ctgctttgat tggtggtcta ggtttggctc catctgccaa cattggtgat
gacaaggctt tattcgaacc tgttcacggt tctgctccag acattgctgg taagggtatt
gccaacccaa tggcttccat cttgtccatt gctatgttgt tcgactacat cggtgaaaag
gaaaagggtg acttgatcag agaagctgtc aaatactgtt tgatcaacaa gaaggttact
ccagatttgg gtggtgactt gaaaaccaag gatgtcggtg acgaaatctt gaactacatc
agaaagaaat tgaaaggcta ctaa
SEQ ID NO: 175
DNA - Artificial
DC-KdcA-F
aaatttggat ccgttgagga ggcctcaaaa atgtatactg ttggtgatta tc
SEQ ID NO: 176
DNA - Artificial
DC-KdcA-R
aaatttggcg cgccattact tgttctgctc cgcaaac
M. maripaludis sequences
SEQ ID NO: 177
AksA CPO E. coli
ATGGACTGGAAAGCGGTATCTCCGTACAACCCGAAACTGAACCTGAAAGACTGCTACCTG
TACGACACCACTCTGCGTGACGGCGAGCAGACTCCGGGCGTTTGCTTCACTCACGACCAG
AAACTGGAAATCGCGAAGAAACTGGACGAACTGAAAATCAAGCAGATCGAAGCTGGCTTC
CCGATCGTTTCTGAAAACGAACGTAAAGCAATCAAGTCTATCACCGGTGAAGGTCTGAAC
GCTCAGATCCTGGCACTCTCTCGCGTACTGAAAGAAGATATCGACAAAGCAATCGAATGC
GACGTTGACGGTATCATCACTTTCATCGCTGCTTCTCCGATGCACCTGAAATACAAACTG
CACAAATCTCTGGATGAAGTTGAAGAGATGGGTATGAAAGCGGTAGAATACGCTAAAGAC
CACGGTCTGTTCGTTGCATTCTCTGCTGAAGATGCAACTCGTACTCCGGTTGAAGATCTG
ATCCGTATCCACAAAAACGCTGAAGAGCACGGTGCTAACCGCGTTCACATCGCTGACACT
CTGGGTTGCGCAACTCCGCAGGCAATGTACCACATCTGCTCTGAACTGTCCTCCAACCTG
AAGAAAGCGCACATCGGTGTTCACTGCCACAACGACTTCGGTTTCGCTGTTATCAACTCC
ATCTACGGTCTGATCGGTGGTGCGAAAGCGGTATCTACTACCGTTAACGGTATCGGTGAA
CGTGCTGGTAACGCTGCTATCGAAGAAATCGTTATGGCGCTGAAAGTTCTGTACGACCAC
GACATGGGTCTGAACACTGAAATCCTGACTGAAATCTCCAAGCTGGTTGAAAACTACTCC
AAGATCCGTATCCCGGAAAACAAGCCGCTGGTTGGTGAAATGGCATTCTACCACGAATCC
GGTATCCACGTTGACGCTGTTCTGGAAAACCCGCTGACTTACGAACCGTTCCTGCCAGAA
AAAATCGGTCAGAAGCGTAAGATCATCCTGGGTAAGCACTCTGGTTGCCGTGCTGTTGCT
CACCGTCTGCAGGAACTGGGTCTGGAAGCATCTCGTGAAGAGCTGTGGGAAATCGTTAAG
AAAACCAAAGAAACTCGTGAAGAAGGTACTGAAATCTCTGACGAAGTATTCAAAAACATC
GTTGACAAAATCATTAAATAA
SEQ ID NO: 178
AksF CPO E. coli
ATGCGTAACACTCCGAAAATCTGCGTTATCAACGGTGACGGTATCGGTAACGAAGTTATC
CCGGAAACCGTTCGCGTACTGAACGAAATCGGTGACTTCGAATTCATCGAAACTCACGCT
GGTTACGAATGCTTCAAGCGCTGCGGTGACGCTATCCCGGAAAAAACTATCGAAATCGCT
AAAGAGTCTGACTCCATCCTGTTCGGTTCTGTAACTACTCCGAAGCCGACTGAACTGAAA
AACAAGCCGTACCGTTCTCCGATTCTGACTCTGCGTAAAGAGCTGGATCTGTACGCTAAC
ATCCGTCCGACTTTCAACTTCAAAAACCTGGACTTCGTTATCATCCGTGAAAACACTGAA
GGTCTGTACGTTAAGAAAGAATACTACGACGAAAAAAACGAAGTTGCAACTGCTGAACGT
ATCATCTCCAAATTCGGTTCTTCCCGTATCGTTAAGTTCGCATTCGACTACGCACTGCAG
AACAACCGTAAGAAAGTTTCCTGCATCCACAAAGCTAACGTTCTGCGTATCACTGACGGT
CTGTTCCTGGGCGTATTCGAAGAAATCTCCAAGAAATACGAGAAGCTGGGTATCGTTTCT
GACGACTACCTGATCGACGCAACTGCGATGTACCTGATCCGTAACCCGCAGATGTTCGAC
GTAATGGTTACCACTAACCTGTTCGGTGACATCCTGTCTGACGAAGCTGCTGGTCTGATC
GGTGGTCTGGGTATGTCCCCGTCTGCTAACATCGGTGACAAAAACGGTCTGTTCGAACCG
GTTCACGGTTCTGCACCGGATATCGCTGGTAAAGGTATCTCCAACCCAATCGCGACTATC
CTGTCTGCTGCAATGATGCTGGATCACCTGAAAATCAACAAAGAAGCTGAATACATCCGT
AACGCTGTTAAGAAAACCGTTGAATGTAAATACCTGACTCCGGACCTGGGTGGTCACCTG
AAAACTTCTGAAGTTACTGAAAAAATCATCGAATCCATCAAATCTCAGATGATTCAGTAA
SEQ ID NO: 179
AksD CPO E. coli
ATGACTCTGGCTGAAAAAATCATCTCCAAAAACGTTGGTAAAAACGTTTACGCTGGCGAC
TCCGTTGAAATCGACGTTGACGTTGCGATGACTCACGACGGTACTACTCCGCTGACCGTT
AAAGCATTCGAGCAGATCTCTGACAAAGTATGGGATAACGAAAAAATCGTTATCATCTTC
GACCACAACATCCCGGCTAACACCTCTAAAGCTGCTAACATGCAAGTTATCACTCGTGAA
TTCATCAAGAAGCAGGGTATCAAAAACTACTACCTGGACGGTGAAGGTATCTGCCACCAG
GTTCTGCCGGAAAAAGGTCACGTTAAGCCGAACATGATCATCGCTGGTGCTGACTCTCAC
ACCTGTACTCACGGTGCATTCGGTGCATTCGCAACTGGCTTCGGTGCAACTGACATGGGT
TACGTTTACGCAACTGGTAAAACCTGGCTGCGCGTACCAGAAACCATTCAGGTTAACGTA
ACTGGCGAAAACGAAAACATCTCCGGTAAAGACATCATCCTGAAAACCTGTAAAGAAGTT
GGTCGTCGCGGTGCAACTTACCTCTCTCTGGAATACGGTGGTAACGCGGTACAGAACCTG
GATATGGACGAACGTATGGTTCTGTCTAACATGGCTATCGAAATGGGTGGTAAAGCGGGT
ATCATCGAAGCTGACGACACCACTTACAAATACCTGGAAAACGCTGGCGTTTCCCGTGAA
GAAATCCTGAACCTGAAGAAAAACAAGATCAAAGTTAACGAATCTGAAGAAAACTACTAC
AAAACTTTCGAGTTCGACATCACTGACATGGAAGAGCAGATCGCTTGCCCGCACCACCCG
GACAACGTTAAAGGCGTTTCTGAAGTTTCTGGTATCGAACTGGATCAGGTATTCATCGGT
TCCTGCACCAACGGTCGTCTGAACGATCTGCGTATCGCTGCGAAGCACCTGAAAGGTAAG
AAAGTTAACGAATCCACTCGTCTGATCGTTATCCCGGCTTCCAAGTCTATCTTCAAAGAA
GCGCTGAAAGAAGGTCTGATCGACACCTTCGTTGACTCCGGTGCGCTGATCTGTACTCCG
GGTTGCGGTCCGTGCCTGGGTGCACACCAGGGCGTACTGGGTGACGGTGAAGTTTGCCTG
GCAACTACCAACCGTAACTTCAAAGGTCGTATGGGTAACACCAAGTCTGAAGTTTACCTC
TCTTCTCCGGCAATCGCTGCGAAGTCTGCTGTTAAAGGTTACATCACTAACGAGTAA
SEQ ID NO: 180
AksE CPO E. coli
ATGAAGATCACCGGTAAAGTTCACGTATTCGGTGACGACATCGACACTGACGCTATCATT
CCGGGTGCTTACCTGAAAACCACTGACGAATACGAACTGGCTTCTCACTGCATGGCGGGT
ATCGACGAAGATTTCCCGGAAATGGTTAAAGAAGGTGACTTCCTGGTTGCTGGCGAAAAC
TTCGGTTGCGGTTCTTCCCGTGAGCAGGCACCGATCGCTATCAAATACTGCGGTATCAAA
GCAATCATCGTTGAATCCTTCGCACGTATCTTCTACCGTAACTGCATCAACCTGGGCGTA
TTCCCGATCGAATGTAAAGGTATCTCCAAGCACGTTAAAGACGGTGACCTGATCGAACTG
GATCTGGAAAACAAGAAAGTTATCCTGAAAGACAAAGTTCTGGACTGCCACATCCCGACT
GGTACTGCGAAAGACATCATGGACGAAGGTGGTCTGATCAACTACGCTAAGAAGCAGAAA
AACTAA
SEQ ID NO: 181
AksA wt
ATGGATTGGAAAGCTGTATCTCCGTACAACCCTAAATTAAATTTGAAAGACTGTTATTTGTAT
GATACGA
CATTGAGAGATGGTGAACAGACTCCCGGAGTTTGTTTTACACATGATCAAAAACTTGAGAT
CGCCAAAAA
ACTGGATGAACTTAAAATTAAACAGATCGAAGCGGGTTTTCCAATTGTTTCTGAAAACGAGA
GAAAAGCC
ATCAAATCAATTACTGGCGAAGGATTAAATGCACAAATTTTGGCGTTATCAAGAGTTTTAAA
AGAGGATA
TTGATAAAGCCATTGAATGTGATGTTGATGGAATAATTACATTCATTGCAGCTTCACCAATG
CATTTGAA
ATACAAATTGCACAAAAGCCTCGATGAAGTCGAAGAAATGGGTATGAAAGCCGTTGAATAC
GCAAAAGAT
CACGGACTTTTCGTAGCATTCTCTGCAGAAGATGCGACAAGAACTCCTGTTGAAGACCTCA
TCAGAATCC
ACAAAAATGCAGAAGAACACGGTGCCAATAGGGTGCATATTGCAGATACCCTCGGGTGTG
CAACACCACA
GGCAATGTATCATATCTGCTCTGAATTAAGCAGTAACTTGAAAAAAGCACATATCGGGGTAC
ACTGTCAC
AACGACTTTGGGTTCGCAGTTATAAACTCGATATACGGATTAATTGGTGGAGCAAAAGCGG
TATCTACAA
CAGTTAACGGAATAGGCGAAAGAGCAGGAAATGCTGCAATTGAAGAAATTGTAATGGCATT
GAAAGTACT
TTACGACCACGATATGGGATTAAATACTGAAATACTAACTGAAATATCGAAACTCGTTGAAA
ACTATTCA
AAAATTAGGATTCCCGAAAATAAACCTCTTGTTGGGGAAATGGCATTTTACCATGAAAGCG
GAATACATG
TTGATGCGGTTTTAGAGAATCCTTTAACGTATGAACCGTTTTTACCTGAAAAAATAGGTCAA
AAAAGAAA
AATTATACTTGGAAAACATTCCGGATGCAGAGCAGTTGCACACAGACTGCAAGAACTTGGG
CTTGAAGCT
TCAAGAGAAGAACTTTGGGAAATTGTGAAAAAAACTAAAGAAACCAGAGAAGAAGGTACTG
AAATAAGCG
ACGAAGTGTTTAAAAACATTGTCGATAAGATTATAAAATAA
SEQ ID NO: 182
AksF wt
ATGAGAAACACTCCCAAAATTTGTGTTATTAATGGAGATGGCATTGGAAACGAAGTGATT
CCTGAAACAGTGCGCGTCTTGAATGAAATTGGGGATTTTGAATTTATAGAAACACATGCG
GGCTACGAATGTTTTAAAAGATGTGGCGATGCGATACCTGAAAAGACCATAGAAATTGCA
AAAGAATCTGATTCTATTCTTTTTGGATCTGTTACTACCCCAAAACCAACTGAATTAAAA
AATAAACCCTATAGAAGTCCAATATTAACTTTAAGAAAAGAACTCGACCTTTATGCAAAT
ATAAGACCGACTTTCAACTTCAAAAACCTTGATTTTGTGATAATTCGCGAAAATACCGAA
GGTCTTTATGTGAAAAAAGAATATTACGACGAAAAAAATGAAGTTGCGACTGCTGAACGA
ATTATTTCTAAATTTGGAAGCTCGAGAATTGTAAAATTTGCTTTTGATTATGCACTTCAA
AACAATAGAAAAAAAGTATCCTGTATTCACAAAGCAAATGTTTTGAGGATCACAGATGGG
TTATTCCTAGGGGTATTTGAAGAAATATCGAAAAAATATGAAAAATTGGGAATAGTGTCT
GATGACTATTTGATTGATGCAACAGCGATGTATTTAATTAGAAATCCGCAAATGTTTGAT
GTCATGGTTACAACAAATTTATTTGGAGATATTTTATCGGATGAAGCTGCTGGACTTATC
GGAGGACTTGGAATGTCTCCTTCAGCAAATATTGGTGACAAAAACGGATTATTCGAACCA
GTGCATGGATCCGCACCAGATATTGCTGGAAAAGGAATTTCAAACCCGATTGCAACAATT
TTAAGTGCTGCAATGATGCTTGATCATTTAAAAATAAATAAAGAAGCGGAATACATAAGA
AATGCAGTTAAAAAAACTGTTGAATGTAAATACCTAACTCCGGATCTTGGGGGACACTTA
AAAACTTCTGAAGTTACAGAAAAAATCATTGAATCAATAAAATCTCAAATGATTCAATGA
SEQ ID NO: 183
AksD wt
ATGACACTTGCTGAAAAAATCATTTCTAAAAATGTTGGAAAAAATGTTTACGCGGGCGAT
AGCGTTGAAATAGACGTGGATGTCGCAATGACGCATGACGGGACTACCCCTCTTACAGTA
AAAGCTTTTGAGCAGATTTCAGACAAAGTTTGGGATAATGAAAAGATAGTTATTATTTTT
GACCACAACATCCCTGCAAACACGTCAAAAGCTGCGAATATGCAGGTTATAACGAGAGAA
TTTATCAAAAAACAGGGAATTAAAAATTATTACCTTGATGGCGAAGGAATATGTCATCAG
GTACTTCCTGAAAAAGGCCACGTGAAGCCAAACATGATAATTGCAGGAGCTGACAGTCAC
ACCTGTACTCATGGGGCATTCGGTGCTTTTGCGACAGGTTTTGGTGCAACTGACATGGGT
TACGTCTATGCAACCGGAAAAACATGGCTTAGAGTTCCTGAAACCATTCAAGTAAATGTA
ACCGGAGAAAATGAAAATATTTCTGGAAAGGACATTATCTTAAAAACTTGTAAGGAAGTT
GGAAGACGTGGAGCGACATACCTGTCTTTAGAATACGGCGGAAATGCAGTCCAAAATCTT
GACATGGACGAAAGAATGGTTTTATCGAACATGGCCATTGAAATGGGCGGAAAAGCTGGA
ATTATCGAAGCTGACGATACTACTTACAAATACCTTGAAAATGCAGGAGTTTCAAGAGAA
GAAATTCTTAACTTGAAAAAAAATAAAATAAAAGTTAATGAATCCGAAGAAAATTACTAC
AAAACATTTGAATTTGATATAACCGATATGGAAGAACAGATTGCTTGCCCGCACCACCCT
GACAATGTAAAAGGAGTTTCTGAAGTATCAGGAATTGAATTAGATCAGGTATTCATCGGA
TCTTGTACAAACGGAAGATTAAACGATTTAAGAATTGCTGCAAAACATTTGAAAGGAAAA
AAAGTTAATGAAAGCACCCGACTAATTGTAATTCCTGCATCAAAATCAATCTTTAAAGAA
GCGTTAAAAGAAGGATTAATCGATACTTTTGTAGATTCTGGAGCATTAATCTGCACTCCT
GGATGCGGACCATGCCTTGGAGCCCATCAGGGTGTTTTAGGTGATGGGGAAGTATGTCTT
GCTACAACCAATAGGAACTTTAAAGGAAGAATGGGAAACACAAAATCGGAAGTTTACCTC
TCATCTCCTGCAATAGCTGCAAAATCCGCAGTTAAAGGATACATTACCAATGAATAA
SEQ ID NO: 184
AksE wt
ATGAAAATAACAGGCAAGGTGCACGTATTTGGGGATGACATCGACACAGATGCGATAATT
CCTGGCGCTTATTTAAAAACAACTGATGAATATGAGCTTGCATCACACTGTATGGCTGGA
ATCGATGAAGATTTTCCAGAAATGGTCAAAGAAGGCGACTTTTTGGTAGCAGGTGAGAAT
TTCGGATGCGGAAGTTCGAGAGAGCAAGCTCCAATTGCAATAAAATACTGCGGAATCAAG
GCAATAATTGTTGAAAGTTTTGCAAGGATATTTTATAGAAATTGTATTAATCTTGGAGTT
TTTCCAATTGAATGCAAAGGAATATCAAAACACGTGAAAGATGGAGATTTAATAGAATTG
GATCTCGAAAATAAAAAAGTAATTTTAAAGGACAAGGTTCTAGACTGCCACATTCCAACC
GGAACTGCAAAAGACATAATGGATGAAGGCGGGCTTATAAATTACGCAAAGAAACAGAAA
AACTAA
SEQ ID NO: 185
wt DNA-sequence (from NCBI)
>gi|111184232|ref|NM_017545.2|Homo sapiens hydroxyacid
oxidase (glycolate oxidase) 1 (HAO1), mRNA
ATGCTCCCCCGGCTAATTTGTATCAATGATTATGAACAACATGCTAAATCAGTACTTCCAAA
GTCTATATATGACTATTACAGGTCTGGGGCAAATGATGAAGAAACTTTGGCTGATAATATTG
CAGCATTTTCCAGATGGAAGCTGTATCCAAGGATGCTCCGGAATGTTGCTGAAACAGATCT
GTCGACTTCTGTTTTAGGACAGAGGGTCAGCATGCCAATATGTGTGGGGGCTACGGCCAT
GCAGCGCATGGCTCATGTGGACGGCGAGCTTGCCACTGTGAGAGCCTGTCAGTCCCTGG
GAACGGGCATGATGTTGAGTTCCTGGGCCACCTCCTCAATTGAAGAAGTGGCGGAAGCTG
GTCCTGAGGCACTTCGTTGGCTGCAACTGTATATCTACAAGGACCGAGAAGTCACCAAGAA
GCTAGTGCGGCAGGCAGAGAAGATGGGCTACAAGGCCATATTTGTGACAGTGGACACACC
TTACCTGGGCAACCGTCTGGATGATGTGCGTAACAGATTCAAACTGCCGCCACAACTCAG
GATGAAAAATTTTGAAACCAGTACTTTATCATTTTCTCCTGAGGAAAATTTTGGAGACGACA
GTGGACTTGCTGCATATGTGGCTAAAGCAATAGACCCATCTATCAGCTGGGAAGATATCAA
ATGGCTGAGAAGACTGACATCATTGCCAATTGTTGCAAAGGGCATTTTGAGAGGTGATGAT
GCCAGGGAGGCTGTTAAACATGGCTTGAATGGGATCTTGGTGTCGAATCATGGGGCTCGA
CAACTCGATGGGGTGCCAGCCACTATTGATGTTCTGCCAGAAATTGTGGAGGCTGTGGAA
GGGAAGGTGGAAGTCTTCCTGGACGGGGGTGTGCGGAAAGGCACTGATGTTCTGAAAGC
TCTGGCTCTTGGCGCCAAGGCTGTGTTTGTGGGGAGACCAATCGTTTGGGGCTTAGCTTT
CCAGGGGGAGAAAGGTGTTCAAGATGTCCTCGAGATACTAAAGGAAGAATTCCGGTTGGC
CATGGCTCTGAGTGGGTGCCAGAATGTGAAAGTCATCGACAAGACATTGGTGAGGAAAAA
TCCTTTGGCCGTTTCCAAGATCTGA
SEQ ID NO: 186
HAOX-5B
>Q9UJM8|HAOX1_HUMAN Hydroxyacid oxidase 1 - Homo sapiens (Human).
Protein sequence
MLPRLICINDYEQHAKSVLPKSIYDYYRSGANDEETLADNIAAFSRWKLYPRMLRNVAET
DLSTSVLGQRVSMPICVGATAMQRMAHVDGELATVRACQSLGTGMMLSSWATSSIEEVAE
AGPEALRWLQLYIYKDREVTKKLVRQAEKMGYKAIFVTVDTPYLGNRLDDVRNRFKLPPQ
LRMKNFETSTLSFSPEENFGDDSGLAAYVAKAIDPSISWEDIKWLRRLTSLPIVAKGILR
GDDAREAVKHGLNGILVSNHGARQLDGVPATIDVLPEIVEAVEGKVEVFLDGGVRKGTDV
LKALALGAKAVFVGRPIVWGLAFQGEKGVQDVLEILKEEFRLAMALSGCQNVKVIDKTLV
RKNPLAVSKI
SEQ ID NO: 187
Optimized DNA sequence:
(optimization done by DNA2.0)
ATGCTGCCACGTCTGATTTGTATTAACGATTACGAACAACACGCGAAGAGCGTACTGCCGA
AATCCATTTACGATTATTACCGTTCTGGTGCAAACGATGAAGAAACGCTGGCTGATAACATC
GCCGCTTTTTCCCGTTGGAAACTGTACCCACGTATGCTGCGTAACGTTGCCGAAACCGACC
TGTCCACCAGCGTCCTGGGTCAGCGTGTGTCCATGCCAATCTGCGTGGGTGCAACCGCAA
TGCAGCGTATGGCACACGTTGACGGCGAACTGGCAACCGTCCGTGCGTGCCAGAGCCTG
GGTACCGGTATGATGCTGAGCAGCTGGGCTACCTCTAGCATCGAGGAAGTGGCAGAAGCT
GGTCCGGAAGCACTGCGCTGGCTGCAGCTGTACATCTACAAAGATCGCGAAGTCACTAAG
AAACTGGTGCGCCAGGCGGAAAAGATGGGTTACAAGGCAATCTTTGTGACTGTTGACACC
CCGTACCTGGGTAACCGCCTGGATGACGTTCGTAACCGCTTCAAGCTGCCGCCGCAGCTG
CGTATGAAGAACTTTGAAACCAGCACCCTGTCCTTTTCCCCAGAAGAAAATTTCGGTGATG
ACTCTGGTCTGGCCGCGTACGTCGCGAAAGCTATCGATCCGTCCATCTCCTGGGAAGATA
TCAAATGGCTGCGTCGTCTGACTTCCCTGCCGATCGTTGCTAAGGGTATTCTGCGTGGTGA
CGACGCGCGTGAAGCTGTTAAACATGGTCTGAACGGCATTCTGGTAAGCAACCATGGCGC
ACGCCAGCTGGATGGTGTACCTGCTACTATTGATGTGCTGCCGGAAATCGTGGAAGCGGT
TGAAGGTAAAGTTGAAGTGTTCCTGGACGGTGGTGTGCGCAAAGGCACCGATGTACTGAA
AGCACTGGCGCTGGGTGCGAAAGCCGTCTTTGTTGGCCGTCCTATTGTTTGGGGTCTGGC
ATTCCAGGGTGAGAAAGGTGTACAGGACGTTCTGGAGATCCTGAAAGAGGAGTTCCGCCT
GGCTATGGCGCTGTCTGGTTGTCAAAACGTGAAAGTAATCGATAAAACCCTGGTACGTAAA
AACCCTCTGGCAGTAAGCAAGATCTAA
SEQ ID NO: 188
LAOX-8C wt DNA-sequence (from NCBI, Acc. D50611)
atgaa taacaatgac attgaatata atgcacctag tgaaatcaag
tacattgatg ttgtcaatac ttacgactta gaagaagaag caagtaaagt ggtaccacat
ggtggtttta actatattgc cggtgcatct ggtgatgagt ggactaaacg cgctaatgac
cgtgcttgga aacataaatt actataccca cgtctagcgc aagatgttga agcgcccgat
acaagtactg aaattttagg tcataaaatt aaagccccat tcatcatggc accaattgct
gcacatggtt tagcccacac tactaaagaa gctggtactg cacgtgcagt ttcagaattt
ggtacaatta tgtccatctc agcttattct ggtgcaacat ttgaagaaat ttctgaaggc
ttaaatggcg gaccccgttg gttccaaatc tatatggcta aagatgacca acaaaaccgt
gatatcttag acgaagctaa atctgatggt gcaactgcta tcatccttac agctgactca
actgtttctg gaaaccgtga ccgtgatgtg aagaataaat tcgtttaccc atttggtatg
ccaattgttc aacgttactt acgtggtaca gcagaaggta tgtcattaaa caatatctac
ggtgcttcaa aacaaaaaat ctcaccaaga gatattgagg aaatcgccgg tcattctgga
ttaccagtat tcgttaaagg tattcaacac ccagaagatg cagatatggc aatcaaacgt
ggtgcatcag gtatctgggt atctaaccac ggtgctcgtc aactatatga agctccaggt
tcatttgaca cccttccagc tattgctgaa cgtgtaaaca aacgtgtacc aatcgtcttt
gattcaggtg tacgtcgtgg tgaacacgtt gccaaagcgc tagcttcagg ggcagacgtt
gttgctttag gacgcccagt cttatttggt ttagctttag gtggctggca aggtgcttac
tcagtacttg actacttcca aaaagactta acacgcgtaa tgcaattaac aggttcacaa
aatgtggaag acttgaaggg tctagattta ttcgataacc catacggtta tgaatactag
SEQ ID NO: 189
LAOX-8C
Q44467_9LACT
>Q44467|Q44467_9LACT Lactate oxidase - Aerococcus viridans.
Protein sequence
MNNNDIEYNAPSEIKYIDVVNTYDLEEEASKVVPHGGFNYIAGASGDEWTKRANDRAWKH
KLLYPRLAQDVEAPDTSTEILGHKIKAPFIMAPIAAHGLAHTTKEAGTARAVSEFGTIMS
ISAYSGATFEEISEGLNGGPRWFQIYMAKDDQQNRDILDEAKSDGATAIILTADSTVSGN
RDRDVKNKFVYPFGMPIVQRYLRGTAEGMSLNNIYGASKQKISPRDIEEIAGHSGLPVFV
KGIQHPEDADMAIKRGASGIWVSNHGARQLYEAPGSFDTLPAIAERVNKRVPIVFDSGVR
RGEHVAKALASGADVVALGRPVLFGLALGGWQGAYSVLDYFQKDLTRVMQLTGSQNVEDL
KGLDLFDNPYGYEY
SEQ ID NO: 190
LAOX-8C Optimized DNA sequence:
(optimization done by DNA2.0)
ATGAACAACAACGACATCGAATATAACGCTCCTTCTGAAATCAAATATATCGACGTGGTTAA
CACCTATGACCTGGAGGAAGAAGCGTCTAAGGTCGTACCGCACGGTGGTTTCAATTACATT
GCAGGTGCCTCTGGTGATGAATGGACCAAACGCGCAAACGATCGTGCATGGAAACACAAA
CTGCTGTATCCGCGCCTGGCCCAGGATGTGGAAGCACCGGATACTTCCACTGAAATCCTG
GGTCACAAAATCAAGGCACCGTTTATTATGGCTCCGATCGCAGCGCACGGCCTGGCACAC
ACCACCAAAGAAGCTGGCACCGCTCGTGCGGTTTCTGAGTTCGGCACCATTATGTCTATCT
CTGCGTATAGCGGTGCCACTTTCGAGGAAATTTCCGAGGGCCTGAACGGTGGCCCGCGTT
GGTTTCAGATTTACATGGCGAAAGATGACCAGCAGAACCGCGATATCCTGGATGAAGCCAA
ATCTGACGGCGCGACTGCTATCATCCTGACCGCGGACTCTACCGTATCCGGTAACCGTGA
CCGTGATGTGAAGAACAAGTTCGTCTATCCTTTCGGTATGCCGATTGTTCAGCGCTATCTG
CGCGGTACCGCTGAGGGTATGAGCCTGAACAACATCTATGGTGCGTCCAAACAGAAAATC
AGCCCACGTGACATCGAAGAAATTGCTGGTCATAGCGGTCTGCCGGTGTTTGTGAAAGGT
ATCCAGCATCCAGAAGATGCGGACATGGCAATCAAACGTGGTGCGTCTGGCATCTGGGTT
AGCAACCACGGTGCGCGTCAGCTGTACGAAGCTCCGGGTAGCTTCGATACCCTGCCGGC
CATCGCGGAACGTGTGAATAAACGCGTGCCGATCGTTTTCGATTCCGGTGTGCGTCGTGG
TGAACATGTGGCAAAAGCACTGGCGTCTGGCGCTGATGTCGTAGCACTGGGCCGTCCAGT
GCTGTTCGGTCTGGCTCTGGGTGGCTGGCAGGGCGCTTACTCCGTCCTGGATTACTTTCA
GAAAGACCTGACCCGTGTTATGCAGCTGACCGGTTCCCAGAACGTAGAGGACCTGAAAGG
CCTGGACCTGTTCGACAACCCTTACGGTTACGAATACTAA
SEQ ID NO: 191
EC 1.1.1.27 - L-lactate dehydrogenases
>Q8NLN0_Corynebacterium glutamicum
MKETVGNKIVLIGAGDVGVAYAYALINQGMADHLAIIDIDEKKLEGNVMDLNHGVVWADSRTRV
TKGTYADCEDAAMVVICAGAAQKPGETRLQLVDKNVKIMKSIVGDVMDSGFDGIFLVASNPVDI
LTYAVWKFSGLEWNRVIGSGTVLDSARFRYMLGELYEVAPSSVHAYIIGEHGDTELPVLSSATIA
GVSLSRMLDKDPELEGRLEKIFEDTRDAAYHIIDAKGSTSYGIGMGLARITRAILQNQDVAVPVS
ALLHGEYGEEDIYIGTPAVVNRRGIRRVVELEITDHEMERFKHSANTLREIQKQFF
SEQ ID NO: 192
EC 1.1.1.28 - D-lactate dehydrogenases
>P52643_Escherichia coli
MKLAVYSTKQYDKKYLQQVNESFGFELEFFDFLLTEKTAKTANGCEAVCIFVNDDGSRPVLEEL
KKHGVKYIALRCAGFNNVDLDAAKELGLKVVRVPAYDPEAVAEHAIGMMMTLNRRIHRAYQRT
RDANFSLEGLTGFTMYGKTAGVIGTGKIGVAMLRILKGFGMRLLAFDPYPSAAALELGVEYVDL
PTLFSESDVISLHCPLTPENYHLLNEAAFEQMKNGVMIVNTSRGALIDSQAAIEALKNQKIGSLG
MDVYENERDLFFEDKSNDVIQDDVFRRLSACHNVLFTGHQAFLTAEALTSISQTTLQNLSNLEK
GETCPNELV
SEQ ID NO: 193
EC 1.1.1.37 - malate dehydrogenases
>P61889_Escherichia coli
MKVAVLGAAGGIGQALALLLKTQLPSGSELSLYDIAPVTPGVAVDLSHIPTAVKIKGFSGEDATP
ALEGADVVLISAGVARKPGMDRSDLFNVNAGIVKNLVQQVAKTCPKACIGIITNPVNTTVAIAAE
VLKKAGVYDKNKLFGVTTLDIIRSNTFVAELKGKQPGEVEVPVIGGHSGVTILPLLSQVPGVSFT
EQEVADLTKRIQNAGTEVVEAKAGGGSATLSMGQAAARFGLSLVRALQGEQGVVECAYVEGD
GQYARFFSQPLLLGKNGVEERKSIGTLSAFEQNALEGMLDTLKKDIALGEEFVNK
SEQ ID NO: 194
>P49814_Bacillus subtilis
MGNTRKKVSVIGAGFTGATTAFLIAQKELADVVLVDIPQLENPTKGKALDMLEASPVQGFDAKIT
GTSNYEDTAGSDIVVITAGIARKPGMSRDDLVSTNEKIMRSVTQEIVKYSPDSIIVVLTNPVDAMT
YAVYKESGFPKERVIGQSGVLDTARFRTFVAEELNLSVKDVTGFVLGGHGDDMVPLVRYSYAG
GIPLETLIPKERIDAIVERTRKGGGEIVNLLGNGSAYYAPAASLTEMVEAILKDQRRVLPTIAYLEG
EYGYEGIYLGVPTIVGGNGLEQIIELELTDYERAQLNKSVESVKNVMKVLS
SEQ ID NO: 195
EC 1.1.1.81 - hydroxypyruvate reductase
>A3LRN9_Pichia stipitis
MTLKQQVLFVGKPNTNTEAYKKFSANFEVINYKITSKSQLIEDFEGRLRYIEAIYAGWGGFDGVG
GFQGEVLRHCPPNVKVVAICSIGHDGYDTEGMSKRGITLTNVPSVIASEAVADLVLYNTLSSFR
NFKMFEKNLGGKLTNTGALRTALVRGEFDQFNGVPVIKPTVGGAFASSCCGRDILSPRGHNVVI
VGFGSIGKLIGERLACIGMNIHYVKRSKLSEQEEASLGYKVTYHATLKDTKNIADLVVIACPGTAH
TRHMVNEEMINDFAKPFRLINIGRGYVVDEKALVNGLQSGKILFAGLDVFENEPSINPDLLNRQD
VVLTPHIGSSTTENFNYTAAAAMFNIETVLYDREDTITRVN
SEQ ID NO: 196
>Q88F00_Pseudomonas putida
MSVDPQKLLRELFDTAIAAAHPRQVLEPYLPADRSGRVIVIGAGKAAAAMAEVVEKSWQGEVS
GLVVTRYGHGANCQKIEVVEAAHPVPDAAGLAVAKRVLELVSNLNEEDRVIFLLSGGGSALLAL
PAEGLTLADKQQINKALLKSGATIGEMNCVRKHLSAIKGGRLAKACWPATVYTYAISDVPGDLA
TVIASGPTVADPSTSADALAILKRYNIEAPKAVIDWLNNPASETVKADDPALARSHFQLIAKPQQ
SLEAAAVKARQAGFSPLILGDLEGESREVAKVHAGIARQIVQHGQPLKAPCVILSGGETTVTVR
GNGRGGRNAEFLLSLTESLKGLPGVYALAGDTDGIDGSEENAGAFMTPASYASAEALGLSASD
ELDNNNGYGYFAALDALIVTEPTRTNVNDFRAILILETAQS
SEQ ID NO: 197
EC 1.1.1.82 - malate dehydrogenases [NADP+]
>Q8NSK9_Corynebacterium glutamicum
MPEVTVNAQQLTVLCTDILTKTGVPAADAHLVGDSLVQADLWGHPSHGVLRLPWYVRRLHSG
AMTTHAHVEVLNDLGAVLALDGHNGIGQVLADHARKEAVTRAMMFGIGAVSVRNSNHFGTAM
YYTRKAAAQGCVSILTTNASPAMAPWGGREKRIGTNPWSIAAPFGETATVVDIANTAVARGKIY
HARQTNMPIPETWAITSEGAPTTDPAEAINGVVLPMAGHKGYAISFMMDVLSGVLTGSQHSTK
VHGPYDPTPPGGAGHLFIALDVAAFRDPQDFDDALSDLVGEVKSTPKAQNTEEIFYPGESEDR
AHRKNSAHGISLPEKTWMELQELAIENHVVTHR
SEQ ID NO: 198
>Q5E5E9_Vibrio fischeri
MKVSYYEVKERLIRKFIASGLAWDDANWVTDVLISSEQRGDKSHGIKHAKNIFDVINSECYIAQA
PIIHDERSITILDGQNSIGPIVAKQAIDIAIKKAKKYGTAAISLRSSNHLFSLSHYVRYIANNNMIGFI
CSSSSPAMAAPNSLNATIGTNPFAFGAPSSKDPIVIDMSSTNVARGKIKEYKDAELDIPVSWALD
EYGNPTTCAIEALKGTLSPLGGYKGFALGCMIDIFSSVLSGSAFSTQITGTSLHMEEADVNKKGD
FLFVLDISKFIQLSEFKIRMDEFIHIIESNGGYIPGTNYINNQFADIEILN
SEQ ID NO: 199
EC 1.1.1.85 - 3-isopropylmalate dehydrogenases
>A9VLG8_Bacillus weihenstephanensis
MEKRIVCLAGDGVGPEIMESAKEVLHMVERLYGHHFHLQDEYFGGAAIDLNGQPLPQRTLAAC
LASDAVLLGAVGGPRWDDAKERPEKGLLALRKGLGVFANVRPVTVESATAHLSPLKNADEIDF
VVVRELTGGIYFSYPKERTEESATDTLTYHRHEIERIVSYAFQLASKREKKVTSIDKANVLESSKL
WRAVTEEVALRYPNVELEHILVDAAAMELIRNPRRFDVIVTENLFGDILSDEASVLAGSLGMLPS
ASHAENGPSLYEPIHGSAPDIAGKNKANPIAMMRSVAMMLGQSFGLTREGYAIEEAISAVLQSG
KCTADIGGNETTTSFTRAVIQEMEEQALVGRGR
SEQ ID NO: 200
>Q5NPQ9_Zymomonas mobilis
MRIALLAGDGIGPEITAEAVKILKAVVGQEIEFDEALIGGAAWKVTGSPLPEETLKLCKNSDAILF
GSVGDPECDHLERALRPEQAILGLRKELDLFANLRPARLFPELQAESPLKENIVTGTDLMIVREL
TGDVYFGTPRGQRKDDQNRREGFDTMRYNEDEVKRIARIGFETARSRSGNLCSIDKSNVLETS
QLWRTVVLEIAQEYPDVELSHMYVDNAAMQLVRAPDQFDVIVTGNLFGDILSDLASACVGSIGL
LPSASLNSEGKGLYEPIHGSAPDIAGLGKANPLATILSGAMMLRYSLKREADADRIEKAVSTALE
KGARTADLGGKMTTSEMGNAVLAALN
SEQ ID NO: 201
EC 1.1.1.93 - tartrate dehydrogenases
>P76251_Escherichia coli
MMKTMRIAAIPGDGIGKEVLPEGIRVLQAAAERWGFALSFEQMEWASCEYYSHHGKMMPDDW
HEQLSRFDAIYFGAVGWPDTVPDHISLWGSLLKFRREFDQYVNLRPVRLFPGVPCPLAGKQPG
DIDFYVVRENTEGEYSSLGGRVNEGTEHEVVIQESVFTRRGVDRILRYAFELAQSRPRKTLTSA
TKSNGLAISMPYWDERVEAMAENYPEIRWDKQHIDILCARFVMQPERFDVVVASNLFGDILSDL
GPACTGTIGIAPSANLNPERTFPSLFEPVHGSAPDIYGKNIANPIATIWAGAMMLDFLGNGDERF
QQAHNGILAAIEEVIAHGPKTPDMKGNATTPQVADAICKIILR
SEQ ID NO: 202
>A2Q846_Aspergillus niger
MTTETTTYRIASIPGDGIGEEVVRATIEVINKLAQTLNTFNIEFTHLPWGTEYYKQHGRYVSEGYL
DTLRQFDAGLFGSVGHPDVPDHVSLWGLLLALRSPLQLYANVRPVRTFPGTKSPLTTAVNGID
WVLVRENSEGEYCGQGGRSHTGQPWEAATEVAIFTRVGVERIMRFAFETARSRPRRHLTVVT
KSNAMRHGMVLWDEVAEEVAKDFPDVTWDKMLVDAMTLRMISKPESLDTIVGTNLHMDILSDL
AAGLAGSIGVAPSSNLDPTRKNPSLFEPVHGSAFDIMGKGVANPVATFWSAAEMLAWLGEKDA
AKKLMDCVEKVCAAGILTPDLGGSANTQGVVDAVCKEIEQQLASS
SEQ ID NO: 203
EC 1.1.2.3 - L-lactate dehydrogenase (cytochrome)
>P00175_Saccharomyces cerevisiae
MLKYKPLLKISKNCEAAILRASKTRLNTIRAYGSTVPKSKSFEQDSRKRTQSWTALRVGAILAAT
SSVAYLNWHNGQIDNEPKLDMNKQKISPAEVAKHNKPDDCWVVINGYVYDLTRFLPNHPGGQ
DVIKFNAGKDVTAIFEPLHAPNVIDKYIAPEKKLGPLQGSMPPELVCPPYAPGETKEDIARKEQL
KSLLPPLDNIINLYDFEYLASQTLTKQAWAYYSSGANDEVTHRENHNAYHRIFFKPKILVDVRKV
DISTDMLGSHVDVPFYVSATALCKLGNPLEGEKDVARGCGQGVTKVPQMISTLASCSPEEIIEA
APSDKQIQWYQLYVNSDRKITDDLVKNVEKLGVKALFVTVDAPSLGQREKDMKLKFSNTKAGP
KAMKKTNVEESQGASRALSKFIDPSLTWKDIEELKKKTKLPIVIKGVQRTEDVIKAAEIGVSGVVL
SNHGGRQLDFSRAPIEVLAETMPILEQRNLKDKLEVFVDGGVRRGTDVLKALCLGAKGVGLGR
PFLYANSCYGRNGVEKAIEILRDEIEMSMRLLGVTSIAELKPDLLDLSTLKARTVGVPNDVLYNE
VYEGPTLTEFEDA
SEQ ID NO: 204
>P33232_Escherichia coli
MIISAASDYRAAAQRILPPFLFHYMDGGAYSEYTLRRNVEDLSEVALRQRILKNMSDLSLETTLF
NEKLSMPVALAPVGLCGMYARRGEVQAAKAADAHGIPFTLSTVSVCPIEEVAPAIKRPMWFQL
YVLRDRGFMRNALERAKAAGCSTLVFTVDMPTPGARYRDAHSGMSGPNAAMRRYLQAVTHP
QWAWDVGLNGRPHDLGNISAYLGKPTGLEDYIGWLGNNFDPSISWKDLEWIRDFWDGPMVIK
GILDPEDARDAVRFGADGIVVSNHGGRQLDGVLSSARALPAIADAVKGDIAILADSGIRNGLDVV
RMIALGADTVLLGRAFLYALATAGQAGVANLLNLIEKEMKVAMTLTGAKSISEITQDSLVQGLGK
ELPAALAPMAKGNAA
SEQ ID NO: 205
EC 1.1.2.4 - D-lactate dehydrogenase (cytochrome)
>P32891_Saccharomyces cerevisiae
MLWKRTCTRLIKPIAQPRGRLVRRSCYRYASTGTGSTDSSSQWLKYSVIASSATLFGYLFAKNL
YSRETKEDLIEKLEMVKKIDPVNSTLKLSSLDSPDYLHDPVKIDKVVEDLKQVLGNKPENYSDAK
SDLDAHSDTYFNTHHPSPEQRPRIILFPHTTEEVSKILKICHDNNMPVVPFSGGTSLEGHFLPTRI
GDTITVDLSKFMNNVVKFDKLDLDITVQAGLPWEDLNDYLSDHGLMFGCDPGPGAQIGGCIAN
SCSGTNAYRYGTMKENIINMTIVLPDGTIVKTKKRPRKSSAGYNLNGLFVGSEGTLGIVTEATVK
CHVKPKAETVAVVSFDTIKDAAACASNLTQSGIHLNAMELLDENMMKLINASESTDRCDWVEKP
TMFFKIGGRSPNIVNALVDEVKAVAQLNHCNSFQFAKDDDEKLELWEARKVALWSVLDADKSK
DKSAKIWTTDVAVPVSQFDKVIHETKKDMQASKLINAIVGHAGDGNFHAFIVYRTPEEHETCSQ
LVDRMVKRALNAEGTCTGEHGVGIGKREYLLEELGEAPVDLMRKIKLAIDPKRIMNPDKIFKTDP
NEPANDYR
SEQ ID NO: 206
>Q5FP89_Gluconobacter oxydans
MPEPVMTASSASAPDRLQAVLKALQPVMGERISTAPSVREEHSHGEAMNASNLPEAVVFAEST
QDVATVLRHCHEWRVPVVAFGAGTSVEGHVVPPEQAISLDLSRMTGIVDLNAEDLDCRVQAGI
TRQTLNVEIRDTGLFFPVDPGGEATIGGMCATRASGTAAVRYGTMKENVLGLTVVLATGEIIRT
GGRVRKSSTGYDLTSLFVGSEGTLGIITEVQLRLHGRPDSVSAAICQFESLHDAIQTAMEIIQCGI
PITRVELMDSVQMAASIQYSGLNEYQPLTTLFFEFTGSPAAVREQVETTEAIASGNNGLGFAWA
ESPEDRTRLWKARHDAYWAAKAIVPDARVISTDCIVPISRLGELIEGVHRDIEASGLRAPLLGHV
GDGNFHTLIITDDTPEGHQQALDLDRKIVARALSLNGSCSGEHGVGMGKLEFLETEHGPGSLS
VMRALKNTMDPHHILNPGKLLPPGAVYTG
SEQ ID NO: 207
EC 1.1.99.2 2-hydroxyglutarate dehydrogenase
>Q9N4Z0 Caenorhabditis elegans
MLNRGTFQVFRGISGPPKKSVDLPKYDLVIVGGGIVGCATARQLLIEKPQLKVALIEKEKELAVH
QSGHNSGVIHAGIYYTPGSLKAKLCVEGLDLSYEFFDKEKVPYKKTGKLIVAVEPEEVPRLDALF
SRAQTNGCRDIEMIDSSKITELEPHCRGLKALWSPHTGIVDWGYVTKRFGEDFEKRGGKIYTSY
PLEKISDNHDPGYPIRVSSGPALAEFETKNLITCAGLQSDRVAALSGCSTDPKIVPFRGEYLLLK
PEKRHLVKTNIYPVPDPRFPFLGVHFTPRMNGDIWLGPNAVLAYKREGYSYFSISPSDLLESLS
YSGMQKLVKKHFTFGIKELYRGVWIAAQVKQLQRFIPELKLSDVTRGPAGVRAQAMDSAGNLV
DDFVFDSGTGKLSPLLMHVRNAPSPAATSSLAIAKMITSEAINRFKL
SEQ ID NO: 208
>Q9VJ28_Drosophila melanogaster
MAQVRLLVQGLRRSLLNVGVAAPNESTATHKRSQHSSSSCGDYDLVVVGGGIVGAASAREIVL
RHPSLKVAVLEKECKLAKHQSGHNSGVIHAGIYYKPGTLKARLCVEGMHLAYAYLDEKKIPYKK
TGKLIVATDEKEVKLLKDLEKRGIANNVPDLRMIEGSEIQEIEPYCQGVMALHSPHTGIVDWGLV
TEHYGQDFKQCGGDIYLDFNVSKFTETKEGTDYPVTIHGAKPGQTVRTKNVLTCGGLQSDLLA
EKTGCPRDPRIVPFRGEYLLLTKEKQHMVKGNIYPVPDPRFPFLGVHFTPRMDGSIWLGPNAV
LALKREGYTWGDINLFELFDALRYPGFVKMASKYIGFGLSEMSKSWFINLQIKALQKYIPDITEYD
IQRGPAGVRAQAMDLDGNLVDDFVFDRGQGSGALAKRVLHCRNAPSPGATSSLAIAKMIADKI
ENEFSIGK
SEQ ID NO: 209
>P13714_Bacillus subtilis
MMNKHVNKVALIGAGFVGSSYAFALINQGITDELVVIDVNKEKAMGDVMDLPHGKAFGLQPVKT
SYGTYEDCKDADIVCICAGANQKPGETRLELVEKNLKIFKGIVSEVMASGFDGIFLVATNPVDILT
YATWKFSGLPKERVIGSGTTLDSARFRFMLSEYFGAAPQNVHAHIIGEHGDTELPVWSHANVG
GVPVSELVEKNDAYKQEELDQIVDDVKNAAYHIIEKKGATYYGVAMSLARITKAILHNENSILTVS
TYLDGQYGADDVYIGVPAVVNRGGIAGITELNLNEKEKEQFLHSAGVLKNILKPHFAEQKVN
SEQ ID NO: 210
>Q88MC4_Pseudomonas putida
MTHPRHALQRSSTMRALLFSSQHYDQESFTKAAGGTALELHFQPARLTLDTAALADGFEVVCA
FINDELDAPVLQRLAAAGTRLIALRSAGYNHVDLAAAQRLGLAVVRVPAYSPHAVAEHAVALILA
LNRRLHRAYNRTREGDFTLHGLTGFDLHGKTVGVVGTGQIGVAFARIMAGFGCQLLAYDPYPN
PELLALGARYLPLPELLREARIISLHCPLTEHTRHLINAQSLAQLQPGAMLINTGRGALVDTPALID
ALKSGQLGYLGLDVYEEEAQLFFEDRSDLPLQDDVLARLLTFPNVIITAHQAFLTREALDAIAATT
LDNINRWAAGNPQNLVMG

1. Method for preparing alpha-ketopimelic acid, comprising converting 2-hydroxyheptanedioic acid into alpha-ketopimelic acid, which conversion is catalysed using a biocatalyst.

2. Method according to claim 1, wherein the biocatalyst comprises an enzyme selected from the group of ‘oxidoreductases acting on the CH—OH group of donors (EC 1.1)’, ‘oxidoreductases acting on the aldehyde or oxo group of donors (EC 1.2)’, enzymes with 2-hydroxypimelate dehydrogenase activity, enzymes with 2-hydroxypimelate oxidase activity, oxidoreductases classified under EC 1.97, and oxidoreductases classified under EC 1.98.

3. Method according to claim 2, wherein said enzyme is selected from the group of
oxidoreductases with oxygen as acceptor (EC 1.1.3), such as a lactate oxidase or another hydroxy acid oxidase;
L-lactate dehydrogenases (EC 1.1.1.27);
hydroxypyruvate reductases, beta-hydroxypyruvate reductases; NADH:hydropyruvate reductases and D-glycerate dehydrogenases (EC1.1.1.81);
malate dehydrogenases [NADP+], NADP+-malic enzymes, NADP+-malic dehydrogenases (nicotinamide adenine dinucleotide phosphate); malate NADP dehydrogenases; NADP+ malate dehydrogenases; NADP+-linked malate dehydrogenase and malate dehydrogenases (NADP+) (EC 1.1.1.82);
3-isopropylmalate dehydrogenases, beta-isopropylmalic enzymes; beta-isopropylmalate dehydrogenases; threo-Ds-3-isopropylmalate dehydrogenases, 3-carboxy-2-hydroxy-4-methylpentanoate:NAD+ oxidoreductases (EC 1.1.1.85);
tartrate dehydrogenases, mesotartrate dehydrogenases (EC 1.1.1.93);
(R)-2-hydroxy-fatty-acid dehydrogenases (EC1.1.1.98);
(S)-2-hydroxy-fatty-acid dehydrogenases (EC 1.1.1.99);
2-oxoadipate reductases (EC 1.1.1.172), 2-ketoadipate reductases, alpha-ketoadipate reductases, 2-ketoadipate reductases
2-hydroxyglutarate dehydrogenase (EC 1.1.99.2); and
D-2-hydroxy-acid dehydrogenase (EC 1.1.99.6).

4. Method according to claim 2, wherein the enzyme originates from an organism selected from the group of Homimidae and Aerococcus; in particular from the group of Homininae, such as from Homo sapiens, and Aerococcus viridans.

5. Method according to claim 1, wherein 2-hydroxyheptanedioic acid is prepared from heptane dioic acid.

6. Method according to claim 5, wherein the preparation of hydroxyheptanedioic acid is catalysed by a biocatalyst comprising an enzyme selected from the group of
oxidoreductases acting on paired donors (with O₂ as oxidant) and incorporation or reduction of oxygen (EC 1.14),
oxidoreductases acting on CH or CH2 groups (EC1.17)
hydrolases (EC 3) with pimelate hydrolase activity' and
hydrolases (EC 3) with pimelate-2-monooxygenase activity.

7. Method according to claim 1, wherein the biocatalyst comprises an enzyme comprising a sequence according to sequence ID 186, sequence ID 189 or a homologue thereof.

8. Method according to claim 5, wherein the heptane dioic acid is prepared using a biocatalyst comprising one or more enzymes of the pimelate synthetic pathway, which one or more enzymes of the pimelate synthetic pathway may in particular be selected from the group of enzymes involved in biosynthesis of pimelyl-CoA, such as Biol, BioZ, BioH, BioW, BioC.

9. Method according to claim 8, wherein the enzyme system is from an organism selected from the group of bacteria, in particular from the group of Eschericia and Bacillus, more in particular from the group of Eschericia coli and Bacillus sphaericus.

10. Method for preparing 6-aminocaproic acid, comprising converting alpha-ketopimelic acid prepared in a method according to claim 1, into 6-aminocaproic acid.

11. Method for preparing adipic acid, comprising biocatalytically decarboxylating alpha-ketopimelic acid prepared in a method according to any of the claims 1-9, thereby forming 5-formylpentanoic acid and converting the 5-formylpentanoic acid into adipic acid, preferably by aldehyde reduction.

12. Method according to claim 1, wherein the method is carried out under fermentative conditions.

13. Heterologous cell, comprising a nucleic acid sequence encoding an enzyme having catalytic activity in the conversion of 2-hydroxyheptanedioic acid into alpha-ketopimelic acid.

14. Heterologous cell according to claim 13, wherein the cell comprises a nucleic acid sequence encoding an enzyme having catalystic activity in the conversion of heptane dioic acid into 2-hydroxyheptanedioic acid.

15. Heterologous cell according to claim 13, comprising at least one nucleic acid sequence encoding an enzyme of the pimelate synthetic pathway of an organism capable of synthesising pimelate.

16. Heterologous cell according to claim 13, comprising at least one nucleic acid sequence encoding an enzyme having catalytic activity with respect to catalysing a reaction step in the preparation of 6-amino caproic acid from alpha-ketopimelic acid or at least one nucleic acid sequence encoding an enzyme having catalytic activity with respect to catalysing a reaction step in the preparation of adipic acid from alpha-ketopimelic acid.

17. Heterologous cell according to claim 13, comprising at least one nucleic acid sequence encoding an enzyme represented by any of the SEQ ID NO's: 186, 189 and homologues thereof.

18. Heterologous cell according to claim 13, wherein the cell is from an organism selected from the group of Escherichia coli, Azotobacter vinelandii, Klebsiella pneumoniae, Anabaena sp., Synechocystis sp., Microcystis aeruginosa, Deinococcus radiourans, Deinococcus geothermalis, Thermus thermophilus, Bacillus sphaericus, Bacillus subtilis, Bacillus amyloliquefaciens, Bacillus methanolicus, Corynebacterium glutamicum, Aspergillus niger, Penicillium chrysogenum, Penicillium notatum, Paecilomyces carneus, Cephalosporium acremonium, Ustilago maydis, Pichia pastoris, Saccharomyces cerevisiae, Kluyveromyces lactis, Candida crucei, Candida maltosa, Yarrowia lipolytica, and Hansenula polymorpha.

19. Use of a heterologous cell according to claim 13 in the preparation of caprolactam, diaminohexane or adipic acid.

20. Nucleic acid comprising a sequence as represented by Sequence ID No: 187, Sequence ID NO: 190 or a non-wild type function analogue thereof.


SEQUENCE LISTING (210 sequences)

SEQ ID NO: 1 - DNA, 1362 bp, Vibrio fluvialis
  CDS (1)..(1362)
atgaacaaaccgcaaagctgggaagcccgggccgagacctattcgctctatggtttcaccgacatgccttcgctgcatcagcgcggcacggtcgtcgtgacccatggcgagggaccctatatcgtcgatgtgaatggccggcgttatctggacgccaactcgggcctgtggaacatggtcgcgggctttgaccacaaggggctgatcgacgccgccaaggcccaatacgagcgttttcccggttatcacgcctttttcggccgcatgtccgatcagacggtaatgctgtcggaaaagctggtcgaggtgtcgccctttgattcgggccgggtgttctatacaaactcggggtccgaggcgaatgacaccatggtcaagatgctatggttcctgcatgcagccgagggcaaaccgcaaaagcgcaagatcctgacccgctggaacgcctatcacggcgtgaccgccgtttcggccagcatgaccggcaagccctataattcggtctttggcctgccgctgccgggctttgtgcatctgacctgcccgcattactggcgctatggcgaagagggcgaaaccgaagagcagttcgtcgcccgcctcgcccgcgagctggaggaaacgatccagcgcgagggcgccgacaccatcgccggtttctttgccgaaccggtgatgggcgcgggcggcgtgattcccccggccaagggctatttccaggcgatcctgccaatcctgcgcaaatatgacatcccggtcatctcggacgaggtgatctgcggtttcggacgcaccggtaacacctggggctgcgtgacctatgactttacacccgatgcaatcatctcgtccaagaatcttacagcgggctttttccccatgggggcggtgatccttggcccggaactttccaaacggctggaaaccgcaatcgaggcgatcgaggaattcccccatggctttaccgcctcgggccatccggtcggctgtgctattgcgctgaaagcaatcgacgtggtgatgaatgaagggctggctgagaacgtccgccgccttgccccccgtttcgaggaaaggctgaaacatatcgccgagcgcccgaacatcggtgaatatcgcggcatcggcttcatgtgggcgctggaggctgtcaaggacaaggcaagcaagacgccgttcgacggcaacctgtcggtcagcgagcgtatcgccaatacctgcaccgatctggggctgatttgccggccgcttggtcagtccgtcgtcctttgtccgccctttatcctgaccgaggcgcagatggatgagatgttcgataaactcgaaaaagcccttgataaggtctttgccgaggttgcctga

SEQ ID NO: 2 - protein, 453 aa, Vibrio fluvialis
MNKPQSWEARAETYSLYGFTDMPSLHQRGTVVVTHGEGPYIVDVNGRRYLDANSGLWNMVAGFDHKGLIDAAKAQYERFPGYHAFFGRMSDQTVMLSEKLVEVSPFDSGRVFYTNSGSEANDTMVKMLWFLHAAEGKPQKRKILTRWNAYHGVTAVSASMTGKPYNSVFGLPLPGFVHLTCPHYWRYGEEGETEEQFVARLARELEETIQREGADTIAGFFAEPVMGAGGVIPPAKGYFQAILPILRKYDIPVISDEVICGFGRTGNTWGCVTYDFTPDAIISSKNLTAGFFPMGAVILGPELSKRLETAIEAIEEFPHGFTASGHPVGCAIALKAIDVVMNEGLAENVRRLAPRFEERLKHIAERPNIGEYRGIGFMWALEAVKDKASKTPFDGNLSVSERIANTCTDLGLICRPLGQSVVLCPPFILTEAQMDEMFDKLEKALDKVFAEVA

SEQ ID NO: 3 - DNA, 1362 bp, Artificial
  Vibrio fluvialis JS17 omega-aminotransferase codon optimised gene
atgaataaaccacagtcttgggaagctcgtgctgaaacctatagcctgtacggctttaccgatatgccgtctctgcaccagcgtggtactgtagtggtaacgcacggtgagggcccgtacatcgtggacgttaatggccgccgttacctggatgcaaacagcggcctgtggaacatggttgcgggcttcgaccacaaaggcctgatcgatgccgcaaaagcgcagtacgaacgcttcccgggttatcacgcgttctttggccgtatgagcgaccagactgtgatgctgagcgaaaaactggttgaagtgtccccgttcgatagcggtcgtgtcttttacactaactctggcagcgaggctaacgataccatggttaagatgctgtggttcctgcacgcagcggaaggcaaacctcagaaacgtaaaattctgacccgttggaacgcttatcacggtgtgactgctgtttccgcatctatgaccggtaaaccgtataacagcgtgttcggtctgccgctgcctggcttcgtgcatctgacctgcccgcactactggcgttatggtgaggaaggcgaaactgaggaacagttcgtggcgcgtctggctcgtgaactggaagaaaccattcaacgcgaaggtgcagatactatcgcgggcttctttgcggagcctgttatgggtgccggcggtgtgattccgccggcgaagggctatttccaggcaatcctgccgatcctgcgcaagtacgacattccggttatttctgacgaagtgatctgcggcttcggccgcaccggtaacacctggggctgcgtgacgtatgacttcactccggacgcaatcattagctctaaaaacctgactgcgggtttcttccctatgggcgccgtaatcctgggcccagaactgtctaagcgcctggaaaccgccatcgaggcaatcgaagagttcccgcacggtttcactgctagcggccatccggtaggctgcgcaatcgcgctgaaggcgatcgatgttgtcatgaacgagggcctggcggaaaacgtgcgccgcctggcgccgcgttttgaagaacgtctgaaacacattgctgagcgcccgaacattggcgaatatcgcggcatcggtttcatgtgggccctggaagcagttaaagataaagctagcaagaccccgttcgacggcaacctgtccgtgagcgaacgtatcgctaatacctgtacggacctgggtctgatctgccgtccgctgggtcagtccgtagttctgtgcccaccatttatcctgaccgaagcgcagatggatgaaatgttcgataaactggagaaagctctggataaagtgttcgctgaagtcgcgtaa

SEQ ID NO: 4 - protein, 406 aa, Methanococcus jannaschii
MTKVLVMFMDFLFENSWKAVCPYNPKLDLKDIYIYDTTLRDGEQTPGVCFTKEQKLEIARKLDELGLKQIEAGFPIVSEREADIVKTIANEGLNADILALCRALKKDIDKAIECDVDGIITFIATSPLHLKYKFNNKSLDEILEMGVEAVEYAKEHGLFVAFSAEDATRTPIEDLIKVHKAAEEAGADRVHIADTTGCATPQSMEFICKTLKENLKKAHIGVHCHNDFGFAVINSIYGLIGGAKAVSTTVNGIGERAGNAALEELIMALTVLYDVDLGLNLEVLPELCRMVEEYSGIKMPKNKPIVGELVFAHESGIHVDAVIENPLTYEPFLPEKIGLKRNILLGKHSGCRAVAYKLKLMGIDYDREMLCEIVKKVKEIREEGKFITDEVFKEIVEEVLRKRNKN

SEQ ID NO: 5 - protein, 391 aa, Methanothermobacter thermautotrophicus
MRYFVSPFNKEAELKFPDRITIYDTTLRDGEQTPGVCLGTEEKLEIARKLDELGIHQIESGFPVVSEQERVSVKSIANEGLNAEILALCRTKKDDIDAAIDCDVDGVITFMATSDLHLKHKLKLTREEALNVCMNSIEYAKDHGLFLAFSAEDATRTDLDFLKQIYRKAENYGADRVHIADTVGAISPQGMDYLVRELRRDIKVDIALHCHNDFGMALSNSIAGLLAGGTAVSTTVNGIGERAGNTSLEELIMALRIIYEVDLGFNIGVLYELSRLVEKHTRMKVPENKPIVGRNVFRHESGIHVDAVIEEPLTYEPFLPEMIGHQRKIVLGKHSGCRAVKAKLEEYGIDVTRDELCRIVEEVKKNREKGKYINDELFYRIVKSVRGPVDF

SEQ ID NO: 6 - protein, 386 aa, Methanococcus maripaludis
MDWKAVSPYNPKLDLKDCYLYDTTLRDGEQTPGVCFAGDQKLEIAKKLDELKIKQIEAGFPIVSENERKAIKSITGEGLNAQILALSRVLKEDIDKAIECDVDGIITFIATSPMHLKYKLHKNLDEVEEMGMKAVEYAKDHGLFVAFSAEDATRTPLEDIIRIHKNAEEHGADRVHIADTLGCATPQAMYHICSELSKHLKKAHIGVHCHNDFGFAVINSIYGLIGGAKAVSTTVNGIGERAGNAAIEEIAMALKVLYDHDMGLNTEILTEISKLVENYSKIKIPENKPLVGEMVFYHESGIHVDAVLENPLTYEPFLPEKIGQKRKIILGKHSGCRAVAHRLQELGLEASRDELWEIVKKTKETREDGTEISDEVFKNIAEKIIK

SEQ ID NO: 7 - protein, 386 aa, Methanococcus maripaludis
MDWKAVSPYNPKLNLKDCYLYDTTLRDGEQTPGVCFTHDQKLEIAKKLDELKIKQIEAGFPIVSENERKAIKSITGEGLNAQILALSRVLKEDIDKAIECDVDGIITFIAASPMHLKYKLHKSLDEVEEMGMKAVEYAKDHGLFVAFSAEDATRTPVEDLIRIHKNAEEHGANRVHIADTLGCATPQAMYHICSELSSNLKKAHIGVHCHNDFGFAVINSIYGLIGGAKAVSTTVNGIGERAGNAAIEEIVMALKVLYDHDMGLNTEILTEISKLVENYSKIRIPENKPLVGEMAFYHESGIHVDAVLENPLTYEPFLPEKIGQKRKIILGKHSGCRAVAHRLQELGLEASREELWEIVKKTKETREEGTEISDEVFKNIVDKIIK

SEQ ID NO: 8 - protein, 386 aa, Methanococcus maripaludis
MDWKAVSPYNPKLDLKDCYLYDTTLRDGEQTPGVCFTHDQKLEIAKKLDELKIKQIEAGFPIVSENERKCIKSITGEGLNAQILALSRVLKEDIDKAIECDVDGIITFIAASPMHLKYKLHKSLDEVEEMGMKAVEYAKDHGLFVAFSAEDATRTPIEDIIRIHKNAEEHGADRVHIADTLGCATPQSMYYICSELSKHLKKAHIGVHCHNDFGFAVINSIYGLLGGAKAVSTTVNGIGERAGNAAIEEIVMALKVLYDYDMGLNTEILTEMSKLVEKYSKIRIPENKPLVGEMAFYHESGIHVDAVLENPLTYEPFLPEKIGQKRKIILGKHSGCRAVAHRLQELGLETSRNELWEIVKKTKETREEGTEISDEVFKNIVDKIIK

SEQ ID NO: 9 - protein, 279 aa, Methanosphaera stadtmanae
MGLSDLHLEVKINKPRDVVNQICMDAIDYGKDHGLFVAFSAEDATRTELPKLLDVYKQAQDHGADRIHIADTTGSINPYATQYLVKNIKKEIDTEIALHCHNDFGFAVANSIAGLFEGATAISTTVNGIGERAGNASLEELIMSLKLLYNKDLGFKTEVIYELSQLVSKYSKIPISDSKAIVGNNVFRHESGIHVDAIVKNPLAYEPFIPEMIGTKRQIVLGKHSGKSAVIEKLDTLNIKVDDTQLSQIVSLVKQERERGEEITNNKFDEILEKVNIKR

SEQ ID NO: 10 - protein, 397 aa, Methanopyrus kandleri
MQSPYVREAVREMDLPDEVIVYDTTLRDGEQTPGVSFTPEQKLEIAHLLDELGVQQIEAGFPVVSEGERDAVRRIAHEGLNADILCLARTLRGDVDAALDCDVDGVITFIATSELHLKHKLRMSREEVLERIADTVEYAKDHGLWVAFSAEDGTRTEFEFLERVYRTAEECGADRVHATDTVGVMIPAAMRLFVAKIREVVDLPIGVHCHDDFGMAVANSLAAVEAGAQAISTTVNGIGERAGNAALEEVIMALKELYGIDPGFNTEVLAELSRKVSEYSGIDVPPNKAVVGENAFRHESGIHVAAVLEEPRTYEPIDPKEVGMNRKIVLGKHTGRKAVVAKLEELGVEPEEEIVEEVLKRIKALGDRRVRVTDSKLEEIVRNVLESRGDRDDPGSR

SEQ ID NO: 11 - protein, 390 aa, Methanobrevibacter smithii
MQYYISHYNKEPELNFPDEITVYDTTLRDGEQTPGVCFSPEEKLEIAKKLDEVKIKQIEAGFPIVSKKEQESVKAITSEGLNAQIISLSRTKKEDIDAALDCDVDGVITFMGTSDIHLEHKMHIGRQEALNTCMNAIEYAKDHGLFVAFSAEDATRTDLDFLKRIYNKAESYGADRVHIADTTGAITPQGITYLVKELKKDVNIDIALHCHNDFGLAVINSISGVLAGANGISTTVNGIGERAGNASLEEVIMSLKLLYGKDLGFKTKHIKELSELVSKASGLPVPYNKPVVGNNVFRHESGIHVDAVIEEPLCYEPYIPELVGQKRQLVLGKHSGCRAVRAKLNECDLDVSDDTLIEIVKKVKKSREEGTYINDDVFKEIVKSCNYKKE

SEQ ID NO: 12 - protein, 386 aa, Methanococcus vannielii
MDWKEVSQYNPKLDLKECYVYDTTLRDGEQTPGVCFTGNQKLEIAKKLDDLGIKQIEAGFPTVSENERKCIKSISSEGLNADILALSRVLKEDIDRAIECDVDGIITFVATSPMHLKYKLHKSFEEVEEMGMKAIEYAKDHGLFVAFSAEDATRTSIENIIKIHKNAEDYGADRVHIADTLGCATPQSMYQICSELNKSLKKAHIGVHCHNDFGFAAINSIYGLMGGAKAVSTTVNGIGERAGNAALEEVVMALKVLYNYDMGLNTELIMETSKLVETYSKIKVPENKPLVGEMVFYHESGIHVDAVLENPLTYEPFLPEKIGQKRKIVLGKHSGCRAVAYRLNELGFEATRDELWEIVKKTKETREQGTEISDEVFKNIVTHILN

SEQ ID NO: 13 - protein, 387 aa, Methanococcus aeolicus
MNWKEVCQYNPKLNLEDCYIYDTTLRDGEQTPGVCFSMEQKLDIAKKLDELGVKQIEAGFPAVSKSEIENVKKIANEGLNAEILALSRALQGDIDKALSCDVDGIITFIAASPLHLKYKLHKSIEEVEEMGMKAVEYAKDHGLFVAFSAEDATRTPIEDLVRIHKNAEEHGADRVHIADTTGCGTPQSIQYICSELSNNLKKAHIGVHCHNDFGLAVINSIYGLLGGAKAASTTVNGIGERAGNAPLEELLLTMNVLYDVKTDLNISIIKELSTMVENYSGIKIPVNKPIVGDKVFYHESGIHVDAVIENPLTYEPFLPERIGQKREIVLGKHSGCSAVESKLKELGLEVPKDRIWDLVKKVKTTREGGEDIDDEMFIKIVDIINKQ

SEQ ID NO: 14 - protein, 420 aa, Methanococcus jannaschii
MTLVEKILSKKVGYEVCAGDSIEVEVDLAMTHDGTTPLAYKALKEMSDSVWNPDKIVVAFDHNVPPNTVKAAEMQKLALEFVKRFGIKNFHKGGEGICHQILAENYVLPNMFVAGGDSHTCTHGAFGAFATGFGATDMAYIYATGETWIKVPKTIRVDIVGKNENVSAKDIVLRVCKEIGRRGATYMAIEYGGEVVKNMDMDGRLTLCNMAIEMGGKTGVIEADEITYDYLKKERGLSDEDIAKLKKERITVNRDEANYYKEIEIDITDMEEQVAVPHHPDNVKPISDVEGTEINQVFIGSCTNGRLSDLREAAKYLKGREVHKDVKLIVIPASKKVFLQALKEGIIDIFVKAGAMICTPGCGPCLGAHQGVLAEGEICLSTTNRNFKGRMGHINSYIYLASPKIAAISAVKGYITNKLD

SEQ ID NO: 15 - protein, 428 aa, Methanothermobacter thermautotrophicus
MVKMNMTEKILAEAAGLREVTPGEIIEARVDLAMTHDGTSPPTIRTFRDIASRGGPARVWDPERIVMVFDHNVPANTIGAAEFQRVTREFAREQGIVNIFQNAAGICHQVLPERGFVRPGMVIVGADSHTCTYGAFGAFATGMGATDMAMVFATGKTWFMVPEAMRIEVTGEPEGHVYAKDVILHIIGEIGVDGATYRSVEFTGDTIESMDVSGRMTICNMAVEMGAKNGIMEPNRQTLDYVRARTGREFRVYSSDEDSQYLEDHHFDVSDLEPQVACPDDVDNVYPVHRVEGTHIDEAFLGSCTNGRYEDLKIAAEVIGDRRVHEDVRFIVSPASREIYLKALEDGIIETFIRAGAIVCNPGCGPCLGAHMGVLAPGEVSIATTNRNFRGRMGDPASSVYLANPAVVAESAIEGVISAPQQEAGNGC

SEQ ID NO: 16 - protein, 418 aa, Methanococcus maripaludis
MTLAEKIISKNVGKNVYAKDSVEISVDIAMTHDGTTPLTVKAFEQISDKVWDNEKIVIIFDHNIPANTSKAANMQVITREFIKKQGIKNYYLDGEGICHQVLPEKGHVKPNMIIAGADSHTCTHGAFGAFATGFGATDMGYVYATGKTWLRVPETIRVNVTGENENISGKDIILKTCKEVGRRGATYMSLEYGGNAVHNLSMDERMVLSNMAIEMGGKAGIIEADDTTYRYLENAGVSREEILELKKNKITVDESEEDYYKTIEFDITGMEEQVACPHHPDNVKGVSEVEGTELNQVFIGSCTNGRLNDLRIAAKYLKGKKVNENTRLIVIPASKSIFKEALNEGLIDIFVDSGALICTPGCGPCLGAHQGVLGDGEVCLATTNRNFKGRMGNTNAQVYLSSPKIAAKSAVKGYITNE

SEQ ID NO: 17 - protein, 418 aa, Methanococcus maripaludis
MTLAEKIISKNVGKNVYAGDSVEIDVDVAMTHDGTTPLTVKAFEQISDKVWDNEKIVIIFDHNIPANTSKAANMQVITREFIKKQGIKNYYLDGEGICHQVLPEKGHVKPNMIIAGADSHTCTHGAFGAFATGFGATDMGYVYATGKTWLRVPETIQVNVTGENENISGKDIILKTCKEVGRRGATYLSLEYGGNAVQNLDMDERMVLSNMAIEMGGKAGIIEADDTTYKYLENAGVSREEILNLKKNKIKVNESEENYYKTFEFDITDMEEQIACPHHPDNVKGVSEVSGIELDQVFIGSCTNGRLNDLRIAAKHLKGKKVNESTRLIVIPASKSIFKEALKEGLIDTFVDSGALICTPGCGPCLGAHQGVLGDGEVCLATTNRNFKGRMGNTKSEVYLSSPAIAAKSAVKGYITNE

SEQ ID NO: 18 - protein, 418 aa, Methanococcus maripaludis
MTLAEKIISKNVGKNVYAGDSVEIDVDIAMTHDGTTPLTVKAFEQISDKVWDNEKIVIIFDHNIPANTSKAANMQVITREFIKKHGIKNYYLDGEGICHQVLPEKGHVKPNMIIAGADSHTCTHGAFGAFATGFGATDMGFVYATGKTWLRVPETIRVNVTGENENISGKDIILKTCKEVGRSGATYMSLEYGGNAVQNLEMNERMVLSNMAIEMGGKAGIIEADDTTYKYLENAGVSREEILNLKKNKITVNESEENYYKTIEFDITDMEEQIACPHNPDNVKGVSEVSGTELDQVFIGSCTNGRLNDLRIAAKYLKGKKVNENTRLIVIPASKSIFAGALKEGLIDIFVESGALICTPGCGPCLGAHQGVLGDGEVCLATTNRNFKGRMGNTKAEVYLSSPKIAAKSAVKGYITNE

SEQ ID NO: 19 - protein, 415 aa, Methanosphaera stadtmanae
MNISEKILAKASNKEEVSPGDTITANIDVAMSHDGTSPPTIKVFEKIADKVWDPEKIVLVFDHVIPANTIGSAEFQQVVREFGKKQKIPNMYIQGEGVCHEVLPDYGHVKPSTVIVGADSHTCTYGAFGAFSTGLGATDLAMVYATGQTWFNVPESLKINVNGTLNENVYSKDVILKIIKELGAYGATYKSLEFHGDTIDNMSVASRLTMTNMAIECGAKNGIMVPNKQTKEYLSQRGITDYTITTASKDAEYEKIYDFDVDDLQPQIACPHNVDNVEDIDKVAGTHIDQAVLGSCTNGRYEDLLQAAEVIEGHKIHEDVELLVFPASRHVYEKAIETGVIQTLLKSNAIICNPGCGPCLGAHMGVMTDDMTCISTTNRNFLGRMGSAKSYVYLSNPAVVAASAIKGEITNPSEI

SEQ ID NO: 20 - protein, 418 aa, Methanopyrus kandleri
MGKTMAEKILSRASGEDAEAGDIVVANIDVAMVHDITGPITVQRLEEMGVERVWDPSKIVVLFDHQVPADSVEAAENHKIMREFVEEQGIEHFYDVREGVCHQVLPEKGHVRPGDVIVGADSHTCTHGALGAFATGIGSTDMAAVFATGKLWFRVPETYRVEITGELPEGVYAKDVVLKVTGEIGADGATYMAIEYHGEVVREMSVSDRMCLCNMAIEMGAKTGMVPPDEKTLEYVKKRAGTEGRPVEPDPDARYEAELTLDVSDLEPQVAKPFSPDNVVPVGEVEGIAIDQVFIGSCTNGRYEDLKVAAEVLEGEEVHDDVRLIVIPASREVYHRTLKDGVLEVLHEAGALICPPNCGPCLGGHMGVLAEGERCVATSNRNFPGRMGHRESEVYLASPATAAASAIEGEITDPRPYL

SEQ ID NO: 21 - protein, 417 aa, Methanobrevibacter smithii
MNITEKILSAKAKKEVTPGEIIEIPVDLAMSHDGTSPPAIKTFEKVATKVWDNEKIAIVFDHNVPANTIGSAEFQKVCRDFIKKQKITKNYIHGDGICHQVLPEKGLVEPGKVIVGADSHTCTYGAYGAFSTGMGATDLAMVYATGKTWFMVPEAIKMEVSGELNSYTAPKDIILKIIGEVGIAGATYKTAEFCGETIEKMGVEGRATICNMAIEMGAKNGIMEPNKEVIQYVSQRTGKKESELNIVKSDEDAQYSEEMHFDITDMEPQIACPNDVDNVKDISKVEGTAVDQCLIGSCTNGRLSDLKDAYEILKDNEINNDTRLLILPASAEIYKQAIHEGYIDAFIDAGAIICNPGCGPCLGGHMGVLSEGETCLSTTNRNFKGRMGDPKSSVYLANSKVVAASAIEGVITNPKDL

SEQ ID NO: 22 - protein, 418 aa, Methanococcus vannielii
MTLAEAILSKKLGKNVYAKDSVEIDVDLAMTHDGTTPLTVKAFEEISDRVFDNKKIVIVFDHNIPANTSKAANMQIITRDFIKKHDIKNYYLDGEGICHQILPEKGHVKPNMVIVGADSHTCTHGAFGAFATGFGASDMGYVYATGKTWFRVPETIRVNVTGKNENISGKDIVLKTCKEVGRSGATYMALEYGGSAVKALNMDERMVLCNMAIEMGGKVGLIEADHTTYDYLKNAGVSNQEIAELQRNKISITENEETYFKTVEFDITDMEEQVACPHHPDNVKGISEVLGTPIDQIFIGSCTNGHIGDLRIAAKILKGKSINKNTRLIVIPASKSILKQALNEGLIDIFVDFGALICAPGCGPCLGAHEGVLGDGEVCLATTNRNFKGRMGNINSEVYLSSPAIAAKSAIKGHITNE

SEQ ID NO: 23 - protein, 421 aa, Methanococcus aeolicus
MTLAEEILSKKVGKKVKAGDVVEIDIDLAMTHDGTTPLSAKAFKQITDKVWDNKKIVIVFDHNVPANTLKAANMQKITREFIKEQNIINHYLDGEGVCHQVLPENGHIQPNMVIAGGDSHTCTYGAFGAFATGFGATDMGNIYATGKTWLKVPKTIRINVNGENDKITGKDIILKICKEVGRSGATYMALEYGGEAIKKLSMDERMVLSNMAIEMGGKVGLIEADETTYNYLRNVGISEEKILELKKNQITIDENNIDNDNYYKIINIDITDMEEQVACPHHPDNVKNISEVKGAPINQVFIGSCTNGRLNDLRIASKYLKGKKVHNDVRLIVIPASKSIFKQALKEGLIDIFVDAGALICTPGCGPCLGAHQGVLGDGEVCLATTNRNFKGRMGNTTAEIYLSSPAIAAKSAIKGYITNE

SEQ ID NO: 24 - protein, 170 aa, Methanococcus jannaschii
MIIKGRAHKFGDDVDTDAIIPGPYLRTTDPYELASHCMAGIDENFPKKVKEGDVIVAGENFGCGSSREQAVIAIKYCGIKAVIAKSFARIFYRNAINVGLIPIIANTDEIKDGDIVEIDLDKEEIVITNKNKTIKCETPKGLEREILAAGGLVNYLKKRKLIQSKKGVKT

SEQ ID NO: 25 - protein, 170 aa, Methanothermobacter thermautotrophicus
MEGIIRGRVWRFGDNVDTDMIIPGRYLRTFSLDELASHVMEGARPEFASQVRKGDIIVAGRNFGCGSSREQAPVALKHAGVVAIIAESFARIFYRNAINIGLPVIMAKVDADDGDEVSIDLRSGQIRNLTAGSEYRMKPFNDYMLSILEDGGLVNHYLKTIDTGISGDEG

SEQ ID NO: 26 - protein, 161 aa, Methanococcus maripaludis
MKITGKVHLFGDDIDTDAIIPGAYLKTTDEYELASHCMAGIDENFPERVEDGDFLVAGENFGCGSSREQAPIAIKYCGIKAIIVESFARIFYRNCINLGVFPIECKGISKHVKDGDVIELDLEEKKVILKDTVLDCNLPTGTAKDIMDEGGLINYAKKQKN

SEQ ID NO: 27 - protein, 161 aa, Methanococcus maripaludis
MKITGKVHVFGDDIDTDAIIPGAYLKTTDEYELASHCMAGIDEDFPEMVKEGDFLVAGENFGCGSSREQAPIAIKYCGIKAIIVESFARIFYRNCINLGVFPIECKGISKHVKDGDLIELDLENKKVILKDKVLDCHIPTGTAKDIMDEGGLINYAKKQKN

SEQ ID NO: 28 - protein, 161 aa, Methanococcus maripaludis
MKITGKVHLFGDDVDTDAIIPGAYLKTTDEYELASHCMAGIDEDFPEMVEEGDFLVAGENFGCGSSREQAPIAIKYCGIKAIIVESFARIFYRNCINLGVFPIECKGISKHVKDGDSIELDLENKKVILKDTVLNCHLPTGTAKEIMDEGGLINYAKKHKN

SEQ ID NO: 29 - protein, 163 aa, Methanosphaera stadtmanae
MDSMKGKVWTFRDCIDTDVIIAGRYLRTFNPEDLAAHVMEAEDPEFSSKVGKGDIIVGGWNFGCGSSREQAPVAIKTAGVSAVIAKSFARIFYRNAINIGLPVITADIEVDEGDILEVNIEDGIIINETTKKTFKIKPFDAEMLDILENGGLVNQYLKNKKEV

SEQ ID NO: 30 - protein, 170 aa, Methanopyrus kandleri
MRDVIRGRAWVFGDDIDTDQIIPGRYLTTQDPEELAKHVMEGADPEFPEKVREGDVIVAGKNFGCGSSREHAPIALKAAGIACVVTRSFARIFYRNAINLGLPLVVCPGVDDAFEDGQGIEVNLREGYVRNLDTGEELEAKPLPDFMMRILEAGGLVELIKREGPRAFEG

SEQ ID NO: 31 - protein, 161 aa, Methanobrevibacter smithii
MDIIKGKTWTFGENIDTDVIIPGRYLRTFNPQDLADHVLEGERPDFTKNVKKGDIIVADENFGCGSSREQAPVAIKTAGVDAIVAKSFARIFYRNAINIGLPVIVCDIQAKDGDIINIDLSKGILTNETTGESVTFEPFKEFMLDILEDNGLVNHYLKEKQ

SEQ ID NO: 32 - protein, 161 aa, Methanococcus vannielii
MKLKGKAHVFSDDVDTDAIIPGAYLRTTDVYELASHCMAGIDENFPKKVNLGDFIVAGENFGCGSSREQAPISIKYLGISAIIAESFARIFYRNSINLGVIPIECKNISKHVKTGDLIELDLENKKIILKDIVLECTVPTGKAKEIIDLGGLINYAKAQMG

SEQ ID NO: 33 - protein, 165 aa, Methanococcus aeolicus
MIIKGNIHLFGDDIDTDAIIPGAYLKTTDPKELASHCMAGIDEKFSTKVKDGDIIVAGENFGCGSSREQAPISIKHTGIKAVVAESFARIFYRNCINIGLIPITCEGINEQIQNLKDGDTIEIDLQNETIKINSMMLNCGAPKGIEKEILDAGGLVQYTKNKLKK

SEQ ID NO: 34 - protein, 347 aa, Methanococcus jannaschii
MMKVCVIEGDGIGKEVIPEAIKILNELGEFEIIKGEAGLECLKKYGNALPEDTIEKAKEADIILFGAITSPKPGEVQNYKSPIITLRKMFHLYANVRPINNFGIGQLIGKIADYEFLNAKNIDIVIIRENTEDLYVGRERLENDTAIAERVITRKGSERIIRFAFEYAIKNNRKKVSCIHKANVLRITDGLFLEVFNEIKKHYNIEADDYLVDSTAMNLIKHPEKFDVIVTTNMFGDILSDEASALIGGLGLAPSANIGDDKALFEPVHGSAPDIAGKGIANPMASILSIAMLFDYIGEKEKGDLIREAVKYCLINKKVTPDLGGDLKTKDVGDEILNYIRKKLKGY

SEQ ID NO: 35 - protein, 331 aa, Methanothermobacter thermautotrophicus
MYRITVIPGDGIGVEVMEAALHVLQALEIEFEFTHAEAGNECFRRCGDTLPEETLKLVRKADATLFGAVTTVPGQKSAIITLRRELDLFANLRPVKSLPGVPCLYPDLDFVIVRENTEDLYVGDEEYTPEGAVAKRIITRTASRRISQFAFQYAQKEGMQKVTAVHKANVLKKTDGIFRDEFYKVASEYPQMEANDYYVDATAMYLITQPQEFQTIVTTNLFGDILSDEAAGLIGGLGLAPSANIGEKNALFEPVHGSAPQIAGKNIANPTAMILTTTLMLKHLNKKQEAQKIEKALQKTLMRGIMTPDLGGTASTMEMAEAIKEEIVKGE

SEQ ID NO: 36 - protein, 339 aa, Methanococcus maripaludis
MRNTPKICVINGDGIGNEVVPETVRVLNELGDFEFIHAHAGYECFKRCGDAIPENTIEIAKESDCILFGSVTTPKPTELKNKSYRSPILTLRKELDLYANIRPTYNFDNLDFVIIRENTEGLYVKKEYYDEKNEVAIAERIISKFGSSRIVKFAFDYAVQNNRKKVSCIHKANVLRVTDGLFLEVFEEMSKHYEKLGIKSDDYLIDATAMYLIRNPQMFDVLVTTNLFGDILSDEAAGLIGGLGMSPSANIGDKNGLFEPVHGSAPDIAGKGISNPIATILSAAMMLDHLKMNKEAEYIRKAVKKTVECKYLTPDLGGNLKTFEVTEKIIESIRSQMIQ

SEQ ID NO: 37 - protein, 339 aa, Methanococcus maripaludis
MRNTPKICVINGDGIGNEVIPETVRVLNEIGDFEFIETHAGYECFKRCGDAIPEKTIEIAKESDSILFGSVTTPKPTELKNKPYRSPILTLRKELDLYANIRPTFNFKNLDFVIIRENTEGLYVKKEYYDEKNEVATAERIISKFGSSRIVKFAFDYALQNNRKKVSCIHKANVLRITDGLFLGVFEEISKKYEKLGIVSDDYLIDATAMYLIRNPQMFDVMVTTNLFGDILSDEAAGLIGGLGMSPSANIGDKNGLFEPVHGSAPDIAGKGISNPIATILSAAMMLDHLKINKEAEYIRNAVKKTVECKYLTPDLGGHLKTSEVTEKIIESIKSQMIQ

SEQ ID NO: 38 - protein, 339 aa, Methanococcus maripaludis
MRNTPKICVINGDGIGNEVIPETVRVLSEIGDFEFIETHAGYECFKRCGDAIPEKTIEIAKESDSILFGSVTTPKPTELKNKPYRSPILTLRKELDLYANIRPTFNFKDLDFVIIRENTEGLYVKKEYYDEKNEVAIAERVISKFGSSRIVKYAFDYALQNNRKKVSCIHKANVLRITDGLFLEVFEEISKKYEKLGIASDDYLIDATAMYLIRNPQMFDVMVTTNLFGDILSDEAAGLIGGLGMSPSANIGDKNGLFEPVHGSAPDIAGKGISNPIASILSAAMMLDHLNMNKEAECIRNAVKKAVECKYLTPDLGGNLKTSEVTDKIIESIKSQMVQ

SEQ ID NO: 39 - protein, 323 aa, Methanosphaera stadtmanae
MYKITVIPGDGIGQEVMQPTIDILETLNSKFEFIPKEAGKECYQKYDTNLPEETIVQCRESDSTLFGAVTSIPQQKSAIVTLRKELDLYVNQRPIHSYTNPDIDFTIIRENSEGLYSHIEESTGDEAIAIRKITYKASERIINYAFNYALKTEKSKVTASHKANVLPVTDGIFKNTFYKVASNYPTIKSNDYYIDAMAMYLITNPAQFDIIVTTNLFGDILSDEGGGLVGTLGLIPSANIGDKTGLFEPVHGSAPDIAGLNKANPIAMILSSCLMLEYLGLYDDAKRIQNAVEETISESKVKTPDMGGHNNTQDVANNILHRL

SEQ ID NO: 40 - protein, 335 aa, Methanopyrus kandleri
MAYKIAVIPGDGIGPEVIEAALHVIEPLIDAEFVEGEAGDECAEKHGDPLPEDTLELCHEADAILFGAAGETAADVIVRLRQELDLYANIRPVRGFPGLRELTGEPYVRDDVDFVIVRENTEGLYSGIEGRFRDTAYTLRIITEEGTRRIAEVACDLAEERGSNTVTCVHKANVMRETCGLFREVCKEVVESRGLEFEEYYVDAAAMFMITEPERFDVVVTPNMFGDILSDEAAALVGGLGLAPSGNVGDRHGLFEPVHGSAPDIAGKGIANPFATILSAVMMLEWLGEDEAAEAVREAVGEAIREGVVTPDLGGDKKTMEVAEFVREAALNRVQ

SEQ ID NO: 41 - protein, 336 aa, Methanobrevibacter smithii
MSTSNKKDNKYQIAVIPGDGIGKEVMEATISVLDELDVDFDYIYGIAGDECNEEHGTPLPQETIDIVRDSDACLFGAAGETAADVIVKIRQEMKMFANLRPVKSYPNTKSLFENVDFMIVRENTEGLYIADQEEETEDGAIAKRVITREAEERIIDYAFQYAKDNNRTKVTAVHKANVLKKTDGLFKKIFYEVGEKYPDIDTEDFYVDATAMYLVTQPQEFQVVVTTNLFGDILSDEGAGLVGGLGLIPSANIGADGALFEPVHGSAPDIAGQQKANPIAMMLSAIMMLRYLGENDAADKFDAAILKVLSEGKTLTGDLGGSATTMEVAQAVKNAL

SEQ ID NO: 42 - protein, 337 aa, Methanococcus vannielii
MGYMPKICVITGDGIGKEVVPETLRVLNEVHDFEYIEAHAGYECFKRCGESIPESTIQTAKNSDSILFGSVTTPKPTELKNKPYRSPILTLRQELDLYANIRPTYNFKDLDFVIIRENTECLYVKREYYDEINEVAIAERIISKKGSERIIKFAFEYARLNNRKKVSCIHKANVLRVTDGLFLEIFEKIAKLYENFGISSNDYLIDATAMYLIKNPYMFDVMVTTNLFGDILSDEAAGLIGGLGMSPSANIGDNLGLFEPVHGSAPDIAGKGISNPIATILSASMMLDHLKMNKKAEIIRNAVKKTINNGYLTPDLGGSLKTSEVVNKVIEFIRDEI

SEQ ID NO: 43 - protein, 343 aa, Methanococcus aeolicus
MKIPKICVIEGDGIGKEVIPETVRILKEIGDFEFIYEHAGYECFKRCGDAIPEKTLKTAKECDAILFGAVSTPKLDETERKPYKSPILTLRKELDLYANVRPIHKLDNSDSSNNIDFIIIRENTEGLYSGVEYYDEEKELAISERHISKKGSKRIIKFAFEYAVKHHRKKVSCIHKSNILRITDGLFLNIFNEFKEKYKNEYNIEGNDYLVDATAMYILKSPQMFDVIVTTNLFGDILSDEASGLLGGLGLAPSANIGDNYGLFEPVHGSAPDIAGKGVANPIAAVLSASMMLYYLDMKEKSRLLKDAVKQVLAHKDITPDLGGNLKTKEVSDKIIEELRKIS

SEQ ID NO: 44 - protein, 440 aa, Saccharomyces cerevisiae
MSENNEFQSVTESTTAPTTSNPYGPNPADYLSNVKNFQLIDSTLREGEQFANAFFDTEKKIEIARALDDFGVDYIELTSPVASEQSRKDCEAICKLGLKAKILTHIRCHMDDARVAVETGVDGVDVVIGTSKFLRQYSHGKDMNYIAKSAVEVIEFVKSKGIEIRFSSEDSFRSDLVDLLNIYKTVDKIGVNRVGIADTVGCANPRQVYELIRTLKSVVSCDIECHFHNDTGCAIANAYTALEGGARLIDVSVLGIGERNGITPLGGLMARMIVAAPDYVRSKYKLHKIRDIENLVADAVEVNIPFNNPITGFCAFTHKAGIHAKAILANPSTYEILDPHDFGMKRYIHFANRLTGWNAIKSRVDQLNLNLTDDQIKEVTAKIKKLGDVRPLNIDDVDSIIKDFHAELSTPLLKPVNKGTDDDNIDISNGHVSKKAKVTK

SEQ ID NO: 45 - protein, 428 aa, Saccharomyces cerevisiae
MTAAKPNPYAAKPGDYLSNVNNFQLIDSTLREGEQFANAFFDTEKKIEIARALDDFGVDYIELTSPVASEQSRKDCEAICKLGLKAKILTHIRCHMDDAKVAVETGVDGVDVVIGTSKFLRQYSHGKDMNYIAKSAVEVIEFVKSKGIEIRFSSEDSFRSDLVDLLNIYKTVDKIGVNRVGIADTVGCANPRQVYELIRTLKSVVSCDIECHFHNDTGCAIANAYTALEGGARLIDVSVLGIGERNGITPLGGLMARMIVAAPDYVKSKYKLHKIRDIENLVADAVEVNIPFNNPITGFCAFTHKAGIHAKAILANPSTYEILDPHDFGMKRYIHFANRLTGWNAIKARVDQLNLNLTDDQIKEVTAKIKKLGDVRSLNIDDVDSIIKNFHAEVSTPQVLSAKKNKKNDSDVPELATIPAAKRTKPSA

SEQ ID NO: 46 - protein, 393 aa, Kluyveromyces lactis
MSVNSNPYAPSPNDLLSNVCNFQLIESTLREGEQFASAFFSTEKKIEIAKALDDFGVDYIELTSPVASEQSRSDCEAICKLGLKAKILTHIRCHMDDARVAVETGVDGVDVVIGTSKFLREYSHGKDMNYIAKSAIEVIEFVKSKGLEIRFSSEDSFRSDIVDLLNIYKTVDKIGVNRVGIADTVGCANPRQVYELVRTLKSVVSCDIECHFHDDTGCAIGNSYSALEAGARLIDVSVLGIGERNGITSLGGLMARMIVSAPEYVKSKYKLHKLRDLENLVADAVSVNVPFNNPITGFCAFTHKAGIHAKAILANPSTYEILNPEDFGMKRYIHFANRLTGWNAIKSRVEQLNLHLSDDQIKEVTSKIKQIGDVRQLSIEDVDTIIKDYHSEL

SEQ ID NO: 47 - protein, 490 aa, Phanerochaete chrysosporium
  misc_feature (62)..(62): Xaa can be any naturally occurring amino acid
LSILVAIQKLEPCCKMCPHANGDSTPNDPSQMVPVDLSNGTSHQASVQSNSNGHAATNGAAXNPYAPRASDFLSNVSNFKIIESTLREGEQFANAFFDTKTKIAIAKALDAFGVEYIELTSPAASEQSRRDCEAICKLGLKAKILTHIRCHMDDARIAVETGVDGVDVVIGTSSFLREFSHGKDMAYITKTAIEVIEFVKSKGIEVRFSSEDSFRSDLVDLLSIYQTVDKIGVNRVGIADTVGCANPRQVYDLVRTLRGVVKCDIEIHLHNDTGMAIANAYTALEAGATHIDTSVLGIGERVGITPLGGLVACLYAANPEYVKSKYNLPMLREIENLVAEAVEVNIPFMNPITGYCAFTHKAGIHAKAILNNPSTYEILKPEDFGLTRYVSIGHRLTGWNAVKSRVEQLGLKLTDEEIKDVTAKIKELADVRTQSMDDVDTLLRVYHSGIQSGELAAGQREALDRLLRKHREGTMSREPSVSRPSTPTQA

SEQ ID NO: 48 - protein, 441 aa, Kluyveromyces lactis
MSSNQDFQPVTESASSVTKFQQNPYGPNPADYLSNVNNYQLIDSTLREGEQFANAFFDTEKKIEIAKALDDFGVDYIELTSPVASEQSRRDCEAICKLGLKAKILTHIRCHMDDARVAVETGVDGVDVVIGTSKFLRQYSHGKDMNYIAKSAIEVIEFVKSKGIEIRFSSEDSFRSDLVDLLNIYKTVDKIGVNRVGIADTVGCANPRQVYELVRTLKSVVSCDIECHFHNDTGCAIANAYTALEGGARLIDVAVLGIGERNGITPLGGLMARMIVAAPEYTKSKYKLHKIRDIENLIAEAVEVNIPFNNPITGFCAFTHKAGIHAKAILANPSTYEILDPHDFGMKRYIHFANRLTGWNAIKSRVDQLNLNLTDDQVKEVTAKIKKLGDIRPLNIDDVDSIIKDFHAEVSTPQLRAVRRDDNDVNDIDIQEPSNKKTKVE

SEQ ID NO: 49 - protein, 418 aa, Schizosaccharomyces pombe
MSVSEANGTETIKPPMNGNPYGPNPSDFLSRVNNFSIIESTLREGEQFANAFFDTEKKIQIAKALDNFGVDYIELTSPVASEQSRQDCEAICKLGLKCKILTHIRCHMDDARVAVETGVDGVDVVIGTSQYLRKYSHGKDMTYIIDSATEVINFVKSKGIEVRFSSEDSFRSDLVDLLSLYKAVDKIGVNRVGIADTVGCATPRQVYDLIRTLRGVVSCDIECHFHNDTGMAIANAYCALEAGATHIDTSILGIGERNGITPLGALLARMYVTDREYITHKYKLNQLRELENLVADAVEVQIPFNNYITGMCAFTHKAGIHAKAILANPSTYEILKPEDFGMSRYVHVGSRLTGWNAIKSRAEQLNLHLTDAQAKELTVRIKKLADVRTLAMDDVDRVLREYHADLSDADRITKEASA

SEQ ID NO: 50 - protein, 465 aa, Aspergillus niger
MCPGADHEPNGQANVANGNGNNGEHPGFTAVETRQNPHPSVSRNPYGHNVGVTDFLSNVSRFQIIESTLREGEQFANAFFDTEKKIEIAKALDEFGVDYIELTSPCASEQSRKDCEAICKLGLKAKILTHIRCHMDDARIAVETGVDGVDVVIGTSSYLREHSHGKDMTYIKNTAIEVIEFVKSKGIEIRFSSEDSFRSDLVDLLSIYSAVDKVGVNRVGIADTVGCASPRQVYELVRVLRGVVSCDIETHFHNDTGCAIANAYCALEAGATHIDTSVLGIGERNGITPLGGLMARMMVADPEYVKSKYRLEKLKDIEDLVAEAVEVNIPFNNYITGFCAFTHKAGIHAKAILNNPSTYEIINPADFGMSRYVHFASRLTGWNAIKSRAQQLKIEMTDDQYKECTAKIKALADIRPIAVDDADSIIRAYYRNLKLGENKPLLDLTADEQAQFAAKEKELAAQASA

SEQ ID NO: 51 - protein, 445 aa, Emericella nidulans
MCPGDHPGFTAVQTRQNPHPSRNPYGHNVGVTDFLSNVSRFKIIESTLREGEQFANAFFDTQKKIEIAKALDEFGVDYIELTSPCASEQSRLDCEAICKLGLKAKILTHIRCHMDDARVAVETGVDGVDVVIGTSSYLREHSHGKDMTYIKNTAIEVIEFVKSKGIEIRFSSEDSFRSDLVDLLSIYSAVDQVGVNRVGIADTVGCASPRQVYELIRVLRGVVSCDIETHFHNDTGCAIANAYCALEAGATHIDTSVLGIGERNGITPLGGLMARMMVADPQYVKSKYKLEKLKDIEDLVAEAVEVNIPFNNYITGFCAFTHKAGIHAKAILNNPSTYEIINPADFGMSRYVHFASRLTGWNAIKSRAQQLNVHMTDDQYKECTAKIKALADIRPIAIDDADSIIRAYYRNLSSGENKPLMDLTADEHAQFLAKEKELTESGTAL

SEQ ID NO: 52 - protein, 474 aa, Penicillium chrysogenum
MVLLPPSLPVCQLKVTAPEFPSNFYLDGDHSGFVGIETRQNPHPSASRNPYGHDAGVTDFLSNVSRFQIIESTLREGEQFANAFFDTAKKIEIAKALDDFGVDYIELTSPCASEQSRADCEAICKLGLKAKILTHIRCHMDDARIAVETGVDGVDVVIGTSSYLREHSHGKDMTYIKNAAIEVIEFVKSKGIEIRFSSEDSFRSDLVDLLSIYSAVDKVGVNRVGIADTVGCASPRQVYELVRVLRGVVGCDIETHFHNDTGCAIANAFCALEAGATHIDTSVLGIGERNGITPLGGLMARMMVADREYVKSKYKLEKLKEIEDLVAEAVEVNIPFNNYITGFCAFTHKAGIHAKAILNNPSTYEIINPADFGMSRYVHFASRLTGWNAIKSRAQQLKLEMTDTQYKECTAKIKAMADIRPIAVDDADSIIRAYHRNLKSGENKPLLDLTAEEQAAFAAKEKELLEAQAAGLPV

SEQ ID NO: 53 - protein, 446 aa, Yarrowia lipolytica
MCATDNAPAANAAPEKPSNVGVEVGHTGEQTNPYGANPADFLSNVSKFQLIESTLREGEQFASAFFDTETKIEIAKALDDFGVDYIELTSPAASEQSRSDCEAICKLGLKAKILTHIRCHMDDARLAVSTGVDGVDVVIGTSQFLRQYSHGKDMNYIAQSAVEVIEFVKSHGIEIRFSSEDSFRSDLVDLLNIYRTVDKIGVNRVGIADTVGCANPRQVYELVRTLKSVVSCDIECHFHNDTGCAIANAYTALEAGANLIDVSVLGIGERNGITSLGGLMARMIAADRDYVLSKYKLHKLRDLENLVADAVQVNIPFNNPITGFCAFTHKAGIHAKAILANPSTYEILNPADFGLTRYIHFANRLTGWNAIKSRVDQLNLHLTDAQCKDVTAKIKKLGDVRSLNIDDVDSIIREFHADVTSTPTVAATEGPAVEDEPAAKKAKTEE

SEQ ID NO: 54 - protein, 687 aa, Phanerochaete chrysosporium
IPQTVIEKVVQKYAVGLPGDKVVKAGDYVMIRPEHVMTHDNTGPVISKFKSIGATRIYNPKQVVFTLDHDVQNKSEKNLKKYATIEAFARTHGIDFYPAGRGIGHQVLVEEGYAFPHTLTVASDSHSNMYGGVGCVGTPIVRTDAAALWATGQTWWQVPRMVKVEFKGRLAPGVSGKDVIVALCGSFNKDEVLNAAIEFSGEGVQHLTVDERLTIANMTTEWGALVGVFPVDDVTLSWYERMLKKLELRTFSTPALGSSIPPPPEHPRINRARLDALRANNLRSDADAEYSSHLVFDLSTLVPYVSGPNSVKVANPLPKLEEAKIKINKAYLLSCTNARASDIAAAAAVIKGHKVHPDVQFYFAPASSEVQREAEQSGDWETLIGAGAKPLPAGCGPCIGLGTGLLEEGEVGISATNRNYKGRMGHPLAQAYLASPAVVAASAIKGYIAGPDSLDPSKLPPAGAPTFSIVNSPSSGAKASQKEPVLVGFPETFAGPLLFAPQDNLNTDGIYPGKYTYQDDITLERQAEVVMENYDPTFAQLDAHTKRGVVLVSGYNFGTGSSREQAATALKSAGIPIVIAGSFGDIFKRNAINNGLVCVESPELVADLTAQFAKDGKRGAGGKEGELTVNKGLSAEVKVVDGALHVTFPDGKTKTYTIQPVGASVQELWLCGGLEGYVLKAIQAENF

SEQ ID NO: 55 - protein, 721 aa, Schizosaccharomyces pombe
MDSGEMHHPYQAFSKVGKCEISQTNPSFSSGMRCLVRSADIQFKGICGLTRGFASFNKPPQTITEKIVQKFAQNIPENKYVRSGDYVTIKPKHCMSHDNSWPVALKFMGIGAKKVFDNRQIVCTLDHDVQNKSEANLRKYKNIESFAKGQGIDFYPAGRGIGHQIMVEQGYAMPGSMAVASDSHSNTYGGVGCLGTPIVRTDAAAIWATGQTWWQIPPIARVNLVGQLPKGLSGKDIIVSLCGAFNHDEVLNHAIEFYGEGLNSLSIESRLTIANMTTEWGALSGLFPTDEKLLAWYEDRLKFLGPNHPRVNRETLDAIKASPILADEGAFYAKHLILDLSTLSPAVSGPNSVKVYNSAATLEKKDILIKKAYLVSCTNGRLSDIHDAAETVKGKKVADGVEFYVGAASSEVEAAAQKNGDWQTLIDSGARTLPAGCGPCIGLGTGLLKDGEVGISATNRNFKGRMGSREALAYLASPAVVAASAIAGKIVAPEGFKNAVSLVSAVDITDKVNKQTASKSSTEAVDSETAIIDGFPSIVAGEIVFCDADNLNTDGIYPGRYTYRDDITKEEMAKVCMENYDSEFGKKTKKDDILVSGFNFGTGSSREQAATAILSRGIPLVVGGSFSDIFKRNSINNALLAIQLPDLVQKLRTAFANESKELTRRTGWHLKWDVRKSTVTVTTSDNKEMSWKIGELGNSVQSLFVRGGLEGWVKHEISKSN

SEQ ID NO: 56 - protein, 693 aa, Kluyveromyces lactis
MFRVQRLRMFSTSRALYAGQNMTEKIVQRHAVGLPEGKTVVSGDYVSIKPAHCMSHDNSWPVALKFMGLGASTIKNPRQVVNTLDHDVQNKSEKNLTKYKNIENFAKKHGIDFYPAGRGIGHQIMIEEGYAFPLTMTVASDSHSNTYGGIGALGTPIVRTDAAAIWATGQTWWQIPPVAQVELKGELPAGISGKDIIVALCGVFNQDQVLNHAIEFTGDSLDKIPIDYRLTIANMTTEWGALSGLFPVDNVLLDFYRNRLTKVGNNHPRINEARINELQAKSDSLQADPDAKYAKKLIIDLSTLTHYVSGPNSVKISSTVDDLSKQDIKVNKAYLVSCTNSRLSDLESAANVVCPSGDINQVHKVAEGVEFYIAAASSEVEAEARATGAWQKLLNAGCLPLPAGCGPCIGLGTGLLEEGQVGISATNRNFKGRMGSKDALAYLASPSVVAASAILGKIGSPAEVLGTKDPNFTGVVATVEDAPATSADGKDVADESGASGSVEILEGFPSEISGELVLCDADNINTDGIYPGKYTYQDDVPKETMAKVCMENYDPDFQTKANPGDILISGFNFGTGSSREQAATAILAKGIKLVVSGSFGNIFFRNSINNALLTLEIPALINMLRDRYKDAPKELTRRTGWFLKWDVSQAKVYVTEGSVNGPIVLEQKVGELGKNLQEIIVKGGLESWVKSQL

SEQ ID NO: 57 - protein, 693 aa, Saccharomyces cerevisiae
MLRSTTFTRSFHSSRAWLKGQNLTEKIVQSYAVNLPEGKVVHSGDYVSIKPAHCMSHDNSWPVALKFMGLGATKIKNPSQIVTTLDHDIQNKSEKNLTKYKNIENFAKKHHIDHYPAGRGIGHQIMIEEGYAFPLNMTVASDSHSNTYGGLGSLGTPIVRTDAAAIWATGQTWWQIPPVAQVELKGQLPQGVSGKDIIVALCGLFNNDQVLNHAIEFTGDSLNALPIDHRLTIANMTTEWGALSGLFPVDKTLIDWYKNRLQKLGTNNHPRINPKTIRALEEKAKIPKADKDAHYAKKLIIDLATLTHYVSGPNSVKVSNTVQDLSQQDIKINKAYLVSCTNSRLSDLQSAADVVCPTGDLNKVNKVAPGVEFYVAAASSEIEADARKSGAWEKLLKAGCIPLPSGCGPCIGLGAGLLEPGEVGISATNRNFKGRMGSKDALAYLASPAVVAASAVLGKISSPAEVLSTSEIPFSGVKTEIIENPVVEEEVNAQTEAPKQSVEILEGFPREFSGELVLCDADNINTDGIYPGKYTYQDDVPKEKMAQVCMENYDAEFRTKVHPGDIVVSGFNFGTGSSREQAATALLAKGINLVVSGSFGNIFSRNSINNALLTLEIPALIKKLREKYQGAPKELTRRTGWFLKWDVADAKVVVTEGSLDGPVILEQKVGELGKNLQEIIVKGGLEGWVKSQL

SEQ ID NO: 58 - protein, 769 aa, Aspergillus niger
MQSRLLPSGPGRRWISLRVPNTPQRRAFASTRFLFQDVFQSQLDDPSSAALFSSLQSSRAVPQTLTEKIVQKYAVGLPDGKFVKSGDYVTIAPHRIMTHDNSWPVALKFMSIGASKMHDPNQVVMTLDHDVQNKTEKNLQKYRQIEEFAKQHGVEFYPAGRGIGHQIMVEEGFAWPGTLVVASDSHSNTYGAVASVGTPIVRTDAASIWATGKTWWQIPPVAKVTFTGILPPGVTGKDVIVALCGLFDKDDVLNHAIEFTGSEETMRSLPMDSRLTIANMTTEWGALSGLFPMDGVLKGWLKGKATTAAMGLADGPFKTLAARNFTHPAIEQLFVNPLTADKGAKYAKELFLDLSTLSPYVSGPNSVKIATPLKELEAQDIKVDKAYLVSCTNSRASDIAAAAKVFKDAAEKNGGKVPKIADGVKFYIAAASIPEQLAAEGAGDWQTLLEAGATALPAGCGPCIGLGTGLLEPGEVGISASNRNFKGRMGSTEAKAYLGSPEIVAASALSGKLSGPGWYQPPEGWTEVVRGEGDGIREEDRMLNTEQALEKLLGQLDDLVADGEKRFAPEEKVEEEGGLTEVYPGFPERVSGEIVFCDADNLNTDAIYPGYWTYQDNVPVEKMAEVCMSNYDKEFASIAKEGDILVVGYNFGCGSSREQAATALLAKQIPLVVSGSFGNIFSRNSINNALMGLEVPRLVSRLREEFGDKQLTRRTGWTLTWDVRRSQIEIQEGQNGPKWTHKVGELPPNVQEIIAKGGLEKWVKNAIEA

SEQ ID NO: 59 - protein, 776 aa, Emericella nidulans
MQSRLVSQSGLGRRWAVLRCALSKTYQRRTLTSTRRQFQDVFQSQLEDPTSAALFSALNSSKAVPQTLTEKIVQKYSVGLPQGKFVKSGDYVTIQPHRCMTHDNSWPCALKFMSIGASRLHNPDQIVMTLDHDVQNKSDKNLKKYRQIEEFATQHGVEFYPAGRGIGHQIMIEEGFAWPGTLAVASDSHSNMYGGVGCLGTPIVRTDAASVWATGKTWWQIPPVAKVTFKGVLPPGVTGKDVIVALCGLFNKDDVLNHAIEFTGSEETMRSLSVDTRLTIANMTTEWGALSGLFPIDSVLKGWLRGKATTAAMGLADGPFKTRAAERFTHPLLEQLFENPLTADKGAKYAKELFLDLSSLSPYVSGPNSVKVATPLKELEAQNIKVDKAYLVSCTNSRASDIAAAAKVFKEAAEKNGGKIPKIADGVKFYIAAASIPEQLAAEGNGDWQTLLEAGATQLPAGCGPCIGMGQGLLEPGEVGISASNRNFKGRMGSTEAKAYLGSPEVVAASALSGKLSGPGWYQTPEGWTEVIRGEGDGIREEDRMLTNEEALEKIIGQLDDLVADGEKRFASETPAVEESEQGLTEIYPGFPERVSGELVFCDADNVNTDGIYPGKYTYQDDVPPETMARVCMENYDPEFSTTAKEGDILVSGFNFGCGSSREQAATAILAKKIPLVVSGSFGNIFSRNSINNALMGLEVPRLVNRLRETFGSGDKVLTRRTGWTLTWDVRKSQIEVQEGPGGPKWTHKVGELPPNVQEIIAKGGLEKWVKNAIGA

SEQ ID NO: 60 - protein, 774 aa, Penicillium chrysogenum
MPSAESGPKTLYDKVFQDHIVNEQEDGTCLIYIDRHLVHEVTSPQAFEGLKNASRQVRRPDCTLATVDHNIPTSSRKNFKNAADFIKENDSRLQCTTLEENVKDFGLTYFGMGDKRQGIVHIIGPEQGFTLPGTTVVCGDSHTSTHGAFGALAFGIGTSEVEHVLATQTLITRRSKNMRIQVDGELPAGVTSKDVVLHIIGVIGTAGGNGAVIEFCGSVIRGLSMEARMSMCNMSIEGGARAGMIAPDEITFEYLKGRPLAPKYGSAEWNKATSYWSSLKSDAGAKYDSEVFIDGKDIIPTISWGTSPQDVVPITGVVPSPDDFEDENRKASCKRALEYMGLVSGTPMKDVVVDKVFIGSCTNARIEDLRAAAKVVNGRKVASNIKRAMIVPGSGLVKEQAESEGLDKVFTDAGFEWREAGCSMCLGMNPDILSPKERCASTSNRNFEGRQGAQGRTHLMSPAMAATAAIVGKLADVREHVVASPVLGKASPKIDVQPVFESPETEDELDRVLDRPADNEPHTNSSAPASGGGKSTGLPTFTTLKGIAAPLDRANVDTDAIIPKQFLKTIKRTGLGTALFYELRYTDDKENPDFVLNQGIYRDSKILVVTGPNFGCGSSREHAPWALLDFGIKCIIAPSFADIFFNNTFKNGMLPVVVSDEVALQKIADEARAGREVEVDLVNQEIKDAQGNKITSFEVEAFRKHCLINGLDDIGLTLQMESKIRSFESKRTLDTPWLDGSAYLRRDRRGATMVEAAPVPKTNRGDVKNEPLEW

SEQ ID NO: 61 - protein, 785 aa, Penicillium chrysogenum
MSPCSMLLKRVARPPVSTTCRLVRPRWAPSFGVPSRTIHHPLRSVSKSLSTRALSTTAPARVEGFHSQHENASIPFSETPSEKRTPQTLTEKIVQRYAVGLPEGKLVRSGDYISLAPGYCMTHDNSWPVALKFMSMGATKIHRPEQIVMTLDHDVQNTSAANLKKYEQIETFAGQHGIDFYPAGRGIGHQVMVEEGYAWPGTMAVASDSHSNHYGGVGCLGTAVVRTDAASIWATSRTWWQIPPVARVTFTGTLPAGVTGKDVIVALCGLFNSDVLNHAIEFTGSEETMESLLVDSRLTIANMTTEWGALTGLFPIDRTLKRWLRYKATEAAMSEDRTTRQRITHERIDELFANPLTADPDAQYAKQLYLNLSTLSPYVSGPNSVKVATPLNELAQQNIKVNRAYIVSCTNSRASDLAAAAKVFKDAAKANPGTTPKIADGVKLYIAAASAPEQEAAESTGDWQALLDAGAQPLPAGCGPCIGLGTGLLEPGEVGISASNRNFKGRMGSRDALAYLASPEVVAASALSGVISGPGAYQVPENWSGVEHGFGTGLPPTTENELTNLLQQMESLIDRVESAGEDSKPATEILPGFPERISGEIVFLDADNLDTDNIYPGKLTYQDNVSKDDMAAACMQNYDPEFKGIAKPSDILVAGFNFGCGSSREQAATAILAKQIPLVVAGSFGNIFSRNSINNALMGLEVPRLIERLRASFAQPPPGDAGRQLTRRTGWTLTWDVKRSVVEVKEGESGESWTEQVGELPANVQEIIAEGGLEAWVKGKVAKSE

SEQ ID NO: 62 - protein, 360 aa, Phanerochaete chrysosporium
MAFRLPLRRALSTAAASRSSLKIGLVPADGIGREVIPAARQAIEALGSDIPKPEFVDLLAGFELFTRTGTALPEETVQALKECDCALFGAVSSPSRRVTGYSSPIVALRKILDLYANVRPVVAPTPEEKPNVDLIVVRENTECLYVKQEQMTPTENGREARATRVITERASRRIGQMAFELAKARPRKHVTIIHKSNVLSITDGLFRETVRSVPRLNEGKYDDVEIAEQLVDSAVYRLFREPHIYDVMVAPNLYGDIISDAAAALVGSLGLVPSVNAGDNFVMGEPVHGSAPDIAGQGIANPIASIRSAALMLRHLGYGAPADRLDKAVDEVIREGQILTPDLGGKSKTQDVVDAVLKRI

SEQ ID NO: 63 - protein, 362 aa, Schizosaccharomyces pombe
MSATRRIVLGLIPADGIGKEVVPAARRLMENLPAKHKLKFDFIDLDAGWGTFERTGKALPERTVERLKTECNAALFGAVQSPTHKVAGYSSPIVALRKKMGLYANVRPVKSLDGAKGKPVDLVIVRENTECLYVKEERMVQNTPGKRVAEAIRRISEEASTKIGKMAFEIAKSRQKIRESGTYSIHKKPLVTIIHKSNVMSVTDGLFRESCRHAQSLDPSYASINVDEQIVDSMVYRLFREPECFDVVVAPNLYGDILSDGAASLIGSLGLVPSANVGDNFVMSEPVHGSAPDIAGRGIANPVATFRSVALMLEFMGHQDAAADIYTAVDKVLTEGKVLTPDLGGKSGTNEITDAVLANIHN

SEQ ID NO: 64 - protein, 360 aa, Emericella nidulans
MAAARTLRIGLIPGDGIGREVIPAGRRILEALPASLNLKFNFVDLDAGYDCFKRTGTALPDKTVEVLKKECDGALFGAVSSPSTKVAGYSSPIVALRKKLDLFANVRPVKTTAGTSAGKPIDLVIVRENTEDLYVKEESTEETPNGKVARAIKQISERASSRIATIAGEIALRRQNIRDGAAASGLRTKPMVTITHKSNVLSQTDGLFRETARAALAAQKFSSVEVEEQIVDSMVYKLFRQPEYYDVIVAPNLYGDILSDGAAALVGSLGLVPSANVGDNFAIGEPCHGSAPDIEGKNIANPIATLRSVALMLEFLGEEQAAAKIYAAVDGNLDEGKYLSPDMGGKATTTEVLEDVLKRL

SEQ ID NO: 65 - protein, 359 aa, Penicillium chrysogenum
MAAARTLRIGLIPGDGIGREVIPAGRRILESLPSSLNLKFSFVDLDAGYETFQKTGTALPDKTVDTLKKECDGALFGAVSSPSTKVAGYSSPIVALRKKLDLYANVRPVKTTAGNSNGKPIDLVIVRENTEDLYVKEERTIEGPNGKVAEAIKRISEKASFRISNIAGEIALRRQNIRAASPTSTRDQPMVTITHKSNVLSQTDGLFRETARRALSAEKFSSVFVEEQIVDSMVYKLFRQPEFYDVIVAPNLYGDILSDGAAALVGSLGLVPSANVGDGFAIGEPCHGSAPDIEGKGISNPIATIRSVALMLEFLGEEKAAAQIYAAVDGNLDAAQFLTPDMGGKATTQQVLDDVLKRL

SEQ ID NO: 66 - protein, 371 aa, Saccharomyces cerevisiae
MFRSVATRLSACRGLASNAARKSLTIGLIPGDGIGKEVIPAGKQVLENLNSKHGLSFNFIDLYAGFQTFQETGKALPDETVKVLKEQCQGALFGAVQSPTTKVEGYSSPIVALRREMGLFANVRPVKSVEGEKGKPIDMVIVRENTEDLYIKIEKTYIDKATGTRVADATKRISEIATRRIATIALDIALKRLQTRGQATLTVTHKSNVLSQSDGLFREICKEVYESNKDKYGQIKYNEQIVDSMVYRLFREPQCFDVIVAPNLYGDILSDGAAALVGSLGVVPSANVGPEIVIGEPCHGSAPDIAGKGIANPIATIRSTALMLEFLGHNEAAQDIYKAVDANLREGSIKTPDLGGKASTQQVVDDVLSRL

SEQ ID NO: 67 - protein, 369 aa, Kluyveromyces lactis
MMRTRFIQLSRRAYASNAKNLTIGLIPGDGIGKEVIPAGKKILESLNPKYGLSFKFIDLQAGWETFQNTGKALPDETIDILKNQCEGALFGAVQSPTTKVEGYSSPIVALRKNLGLFANVRPVKSVDGTKDRKVDLVIVRENTEDLYIKLEKSYIDEATGTRVADATKRITEIATKNIATIALQIAQQRLEQNGHATLTVTHKSNVLSQSDGLFREVCRETYEANKDKYGGVQYNEQIVDSMVYRMFREPECFDVVVAPNLYGDILSDGAAALVGSLGVVPSANVGPNIVIGEPCHGSAPDIAGKGISNPIATIRSTALMLEFLGYPEPAKDIHKAVDANIREGKYLTPDLGGNSTTQQVLEDVLSKLD

SEQ ID NO: 68 - protein, 536 aa, Penicillium chrysogenum
MSPPTALDVNLVGVTDTSTVPVPEPLTVNGVSAWREKTAKVPTGVAAACNSDMFKSPICYTKPKAKQFEHRFSLEAKSRKASTLKTAARYLKTPGLISLGGGLPSPEYFPFEHLDIKVPTAPGFSPEATRESGTVLRAGKHDIQEGTSTYDLEIALNYGQATGAAPLLRFVTEHTEIIHSPPYSDWQCTLTAGSTYAWDTALRVFCERGDYILMEEYTFASAAETAFPLGIKVAGIPMDEQGLIPEAMDKILGDWDVAARGARKPHVLYTIPTGQNPTGATQSAERRHAVYKVAQKHDLIIVEDEPYYFLQMQPYTSGDASPVPPPSSHEEFINSLVPSFLSMDTDGRVVRLESFSKVISPGSRVGWIVASEQIIERFIRNFEVSSQNPSGIAQIALFKLLDEHWGHSGYLDWLINLRMSYTARRDSLVHACEKHLPREIVHWEAPAAGMFQWMSIDWRKHPGIAAGKTHADIEEEIFLSAVNGGVLLSRGSWFKPDHDTVEEKMFFRATFAAASSEKIDEAISRFAQSLRAQFGL

SEQ ID NO: 69 - protein, 376 aa, Thermus thermophilus
MREWKIIDSTLREGEQFEKANFSTQDKVEIAKALDEFGIEYIEVTTPVASPQSRKDAEVLASLGLKAKVVTHIQCRLDAAKVAVETGVQGIDLLFGTSKYLRAAHGRDIPRIIEEAKEVIAYIREAAPHVEVRFSAEDTFRSEEQDLLAVYEAVAPYVDRVGLADTVGVATPRQVYALVREVRRVVGPRVDIEFHGHNDTGCAIANAYEAIEAGATHVDTTILGIGERNGITPLGGFLARMYTLQPEYVRRKYKLEMLPELDRMVARMVGVEIPFNNYITGETAFSHKAGMHLKAIYINPEAYEPYPPEVFGVKRKLIIASRLTGRHAIKARAEELGLHYGEEELHRVTQHIKALADRGQLTLEELDRILREWITA

SEQ ID NO: 70 - protein, 393 aa, Deinococcus radiodurans
MAGIFMTDAPPPLIPARSWAIIDSTLREGEQFARGNFGTDDKVEIARALDAFGAEYIEVTTPMVSEQTRQDIRKLTGLGLRAKFLTHVRCHMEDVQRAVDTGVDGLDLLFGTSSFLREFSHGKSIAQIIDTAGEVIGWIKTHHPELEIRFSAEDTFRSEEADLMAVYSAVSELGVHRVGLADTVGVATPRQVYTLVREVRKVIHEGCGIEFHGHNDTGCAVSNAYEAIEAGATHIDTTILGIGERNGITPLGGLLARMFTFDPQGLIDKYNLELLPELDRMIARMVDLPVPWNNYLTGEFAYNHKAGMHLKAIYLNPGAYEAIPPGVFGVGRRIQAASKVTGKHAIAYKARELGLHYGEDALRRVTDHIKSLAEQDELDDAHLEQVLREWVSA

SEQ ID NO: 71 - protein, 389 aa, Deinococcus geothermalis
MTPDSSTPLIPARSWAIIDSTLREGEQFARGNFKTGDKIEIARLLDAFGAEFLEVTTPMVGAQTQADIRRLTSLGLNAKILTHVRCHLEDVQRAVDLGVDGLDLLFGTSSFLREFSHGKSIAQIIDTASEVIGWIKQNHPDLEIRFSAEDTFRSEEADLMAVYRAVSDLGVHRVGLADTVGVATPRQVYTLVREVRKVIHAECGIEFHGHNDTGCAVSNAYEAIEAGATHIDTTILGIGERNGITPLGGFLARMFTFDPQGLIDKYNLELLPELDRLIARLVDLPIPWNNYLTGEFAYNHKAGMHLKAIYLNPGAYEAIPPSVFGVGRRIQAASKVTGKHAIAHKARELGLHYGEDALRRVTDHIKALAEEGELDDAHLEQVLREWVRA

SEQ ID NO: 72 - protein, 553 aa, Sulfolobus solfataricus
MALKMKYDFLLLSLKLLNLPIIFHLCSVSKKSVEVLDTTLRDGSQGANISFTLNDKIKIALLLDELGVDYIEGGWPGSNPKDEEFFREIKKYRLSKAKIAAFGSTKRKDVSVKEDISLNSIVKADVDVAVIFGKSWSLHATEVLKVTKQDNLDIVYDSINYLKSHGLKVIFDAEHFYQGFKEDPEYALEVVKTAESAGADVIALADTNGGTPPFEVYEITKKVREVLQVKLGIHAHNDIGCAVANSLMAIKAGARHVQGTINGIGERTGNADLIQIIPTLILKMGLNALNGQESLRKLREVSRIVYEILGLPPNPYQPYVGDNAFAHKAGVHVDAVMKVPRAYEHVDPSLVGNDRKFVISELSGTANLVSYLQGLGIAVDKKDERLKKALNKIKELEARGYSFDVGPASAILITLKELNIYKNYINLEYWKVINENNGLSIGIVKVNSQLEVAEGVGPVNAIDRALRMALQRVYPEIGEVKLIDYRVILPSEIKNTESVVRVTIEFTDNKMNWRTEGVSKSVVEASVMALVDGLDYYLQLKKTLKTAVDNYIV

SEQ ID NO: 73 - protein, 361 aa, Thermococcus kodakarensis
MVLDSTLREGEQTPGVNFSPEDRLRIGIALDEVGVDFIEAGHPAVSGEILEGIRLLASHGLNANILAHSRALRSDIDLVLKAEAEWIGIFMCLSQRCLERRFRTDLSGALTRVEDAILYAKDHGLKIRFTPEDTTRTEWKNLTAALNLARELKVDRVSIADTTGAAHPLEFYDLVKRVVEFGIPVNVHCHNDLGLALANAIMGIEAGATLVDATVNGIGERAGIVDLSHLLAALYYHYGVKKYRLEKLYSLSRLVSEITGLQVQVNYPIVGQNAFTHKAGLHVSAVVRDPSFYEFLPAETFGRERTIYVDRFAGRETIRFHLSRFGIHDEEIIEELLRRVKASRRPFTPEMLAEEARRMMT

SEQ ID NO: 74 - protein, 361 aa, Pyrococcus horikoshii
MILDSTLREGEQTPGVNYSPEQRLRIALALDEIGVDFIEVGHPAVSKDVFIGIKLIASQDLNANLLAHSRALLEDIDYVIQADVEWVGIFFCLSNACLRKRFRMSLSQALERISKAIEYAKDHGLKVRFTPEDTTRTEWENLRRAIELAKELKVDRISVADTTGGTHPLRFYTLVKKVVNFGIPVNVHCHNDLGLALANAIMGIEGGATVVDATVNGLGERAGIVDLAQIVTVLYYHYGVKKYRLDKLYEISRMVSEITGIALQPNYPIVGENAFTHKAGLHVSAVLKDPRFYEFLPAEVFGRERTIYVDRFAGKDTIRYYLQKLGINDEEFVKVLLKRVKSSREPFTWDKFIEEVRRLKT

SEQ ID NO: 75 - protein, 385 aa, Azotobacter vinelandii
MASVIIDDTTLRDGEQSAGVAFNADEKIAIARALAELGVPELEIGIPSMGEEEREVMHAIAGLGLSSRLLAWCRLCDVDLAAARSTGVTMVDLSLPVSDLMLHHKLNRDRDWALREVARLVGEARMAGLEVCLGCEDASRADLEFVVQVGEVAQAAGARRLRFADTVGVMEPFGMLDRFRFLSRRLDMELEVHAHDDFGLATANTLAAVMGGATHINTTVNGLGERAGNAALEECVLALKNLHGIDTGIDTRGIPAISALVERASGRQVAWQKSVVGAGVFTHEAGIHVDGLLKHRRNYEGLNPDELGRSHSLVLGKHSGAHMVRNTYRDLGIELADWQSQALLGRIRAFSTRTKRRSPQPAELQDFYRQLCEQGNPELAAGGMA

SEQ ID NO: 76 - protein, 381 aa, Klebsiella pneumoniae
MERVLINDTTLRDGEQSPGVAFRTSEKVAIAEALYAAGITAMEVGTPAMGDEEIARIQLVRRQLPDATLMTWCRMNALEIRQSADLGIDWVDISIPASDKLRQYKLREPLAVLLERLAMFIHLAHTLGLKVCIGCEDASRASGQTLRAIAEVAQNAPAARLRYADTVGLLDPFTTAAQISALRDVWSGEIEMHAHNDLGMATANTLAAVSAGATSVNTTVLGLGERAGNAAAWKPSALGLERCLGVETGVHFSALPALCQRVAEAAQRAIDPQQPLVGELVFTHESGVHVAALLRDSESYQSIAPSLMGRSYRLVLGKHSGRQAVNGVFDQMGYHLNAAQINQLLPAIRRFAENWKRSPKDYELVAIYDELCGESALRARG

SEQ ID NO: 77 - protein, 381 aa, Pseudomonas stutzeri
MSIVIDDTTLRDGEQSAGVAFSAEEKLAIARALAQLGVPELEIGIPSMGEEECEVMRAIAGLALPVRLLAWCRLCDADLLAAGGTGVGMVDLSLPVSDLMLQHKLGRDRDWALREAARLVGAARDAGLEVCLGCEDASRADPEFIVRVAEVAQAAGARRLRFADTVGVMEPFAMHARFRFLAERLDLELEVHAHDDFGLATANTLAAVRGGATHINTTVNGLGERAGNAALEECALALKHLHGIDCGIDVRGIPSISALVEQASGRQVAWQKSVVGAGVFTHEAGIHVDGLLKHRRNYEGLNPDELGRSHSLVLGKHSGAHMVELSYRELGIELQQWQSRALLGCIRRFSTQTKRSPQSADLQGFYQQLCEQGLALAGGAA

SEQ ID NO: 78 - protein, 477 aa, Acinetobacter sp.
MNYPNIPLYINGEFLDHTNRDVKEVFNPVNHECIGLMACASQADLDYALESSQQAFLRWKKTSPITRSEILRTFAKLAREKAAEIGRNITLDQGKPLKEAIAEVTVCAEHAEWHAEECRRIYGRVIPPRNPNVQQLVVREPLGVCLAFSPWNFPFNQAIRKISAAIAAGCTIIVKGSGDTPSAVYAIAQLFHEAGLPNGVLNVIWGDSNFISDYMIKSPIIQKISFTGSTPVGKKLASQASLYMKPCTMELGGHAPVIVCDDADIDAAVEHLVGYKFRNAGQVCVSPTRFYVQEGIYKEFSEKVVLRAKQIKVGCGLDASSDMGPLAQARRMHAMQQIVEDAVHKGSKLLLGGNKISDKGNFFEPTVLGDLCNDTQFMNDEPFGPIIGLIPFDTIDHVLEEANRLPFGLASYAFTTSSKNAHQISYGLEAGMVSINHMGLALAETPFGGIKDSGFGSEGGIETFDGYLRTKFITQLN

SEQ ID NO: 79 - protein, 473 aa, Brucella melitensis
MRIGKMEMQTRYPDVKLFIDGTWRDGSRGETIEIFNPATDEVIGHIARATTADLDDALAAVDRGFEAWSKVSAFDRYKIMRRAADIFRSRGEEVARLLTMEQGKPLAEARIEAAAACDLIDWFAEEARRSYGRIVPPRQAYVMQAEVKEPVGPVAAFTPWNFPINQAVRKISAALAAGCSILLKAAEDTPAAPAELVRAFAEAGLPDGAINLVYGDPAEISAYLIPHPVIRKVSFTGSTQVGKQLAALAGLHMKRVTMELGGHAPVIIAADADVEQAIKVVSGSKFRNAGQVCISPTRFLIENSVYDQVVEGMAAYATSLKVGDGLEAGTTMGPLVNAKRVNAMERLVQDAREHKARVVTGGERIGNRGNFFEPTILADVPRDAAIMNEEPFGPVALLNRFDALDEALSEANRLNYGLAAYAFTGSSAKAARISSTVRSGMITINQLRSGPAGSALRRDQRFRLWNGRRCRRA

SEQ ID NO: 80 - protein, 530 aa, Acinetobacter baumannii
MRLIMLNITGQNFIAGQRSSAGSKFVLSYDAATDEALPYQFAQATPEEIDQAAQAAALAYPAFRQTTPEQRAVFLETIASEIDALDDQFIATVCQETALPEARIRGERGRTTGQLRLFAQVLRRGDYLGARIDLALPERQPLPRPDLRQYKIGVGPVAVFGASNFPLAFSTAGGDTASALAAGCPVIVKAHSGHMATAESIANAICSAIEKCAMPKGIFSMIYGQGVGEPLVKHPAIKAVGFTGSLKGGRALCDLAAARPEPIPVFAEMSSINPMILLPEALKVRGDKIATELSGSVVLGCGQFCTNPGLIIGIKSPEFSQFLDHFKAAMAQQPPQTMLNKGTLRSYEHGLKELLAHDKIEHLAGQPQQGPQAYPQLFKADVSLLLEHDEFLQEEVFGPTTIVIEVESAEQLALALNGLRGQLTASLIAEPQDFENFATLIPLLEEKAGRLLLNGYPTGVEVCDAMVHGGPYPATSDARGTSVGTLAIERYLRPVCYQNYPDHLLPLALQNANPLGIARLVNGEMSKAAL

SEQ ID NO: 81 - protein, 481 aa, Azospirillum brasilense
MANVTYTDTQLLIDGEWVDAASGKTIDVVNPATGKPIGRVAHAGIADLDRALAAAQSGFEAWRKVPAHERAATMRKAAALVRERADAIAQLMTQEQGKPLTEARVEVLSAADIIEWFADEGRRVYGRIVPPRNLGAQQTVVKEPVGPVAAFTPWNFPVNQVVRKLSAALATGCSFLVKAPEETPASPAALLRAFVDAGVPAGVIGLVYGDPAEISSYLIPHPVIRKVTFTGSTPVGKQLASLAGLHMKRATMELGGHAPVIVAEDADVALAVKAAGGAKFRNAGQVCISPTRFLVHNSIRDEFTRALVKHAEGLKVGNGLEEGTTLGALANPRRLTAMASVIDNARKVGASIETGGERIGSEGNFFAPTVIANVPLDADVFNNEPFGPVAAIRGFDKLEEAIAEANRLPFGLAGYAFTRSFANVHLLTQRLEVGMLWINQPATPWPEMPFGGVKDSGYGSEGGPEALEPYLVTKSVTVMAV

SEQ ID NO: 82 - DNA, 1350 bp, Bacillus weihenstephanensis
  CDS (1)..(1350)
gtgcaagcgacggagcaaacacaaagtttgaaaaaaacagatgaaaagtacctttggcatgcgatgagaggagcagcccctagtccaacgaatttaattatcacaaaagcagaaggggcatgggtgacggatattgatggaaaccgttatttagacggtatgtccggtctttggtgcgtgaatgttgggtatggtcgaaaagaacttgcaagagcggcgtttgaacagcttgaagaaatgccgtatttccctctgactcaaagtcatgttcctgctattaaattagcagaaaaattgaatgaatggcttgatgatgaatacgtcattttcttttctaacagtggatcggaagcgaatgaaacagcatttaaaattgctcgtcaatatcatcaacaaaaaggtgatcatggacgctataagtttatttcccgctaccgcgcttatcacggtaactcaatgggagctcttgcagcaacaggtcaagcacagcgaaagtataaatatgaaccactcgggcaaggattcctgcatgtagcaccgcctgatacgtatcgaaatccagaggatgttcatacactggcaagtgctgaggaaatcgatcgtgtcatgacatgggagttaagccaaacagtagccggtgtgattatggagccaatcattactgggggcggaattttaatgcctcctgatggatatatgggaaaagtaaaagaaatttgcgagaagcacggtgcgttgctcatttgtgatgaagttatatgtggatttggccggacagggaagccatttggatttatgaattatggcgtcaaaccagatatcattacaatggcaaaaggtattacaagtgcgtatcttcctttgtcagcaacagcagttagacgagaggtttatgaggcattcgtaggtagtgatgattatgatcgcttccgccatgtaaatacgttcggagggaatcctgctgcttgcgctttagctttgaagaatttagaaattatggagaatgagaaactcattgaacgttccaaagaattgggtgaacgactgttatatgagctagaggatgtaaaagagcatccaaacgtaggggatgttcgcggaaagggccttcttttaggcattgaactagtggaagataagcaaacaaaagaaccggcttccattgaaaagatgaacaaagtcatcaatgcttgtaaagaaaaaggtctaattattggtaaaaatggtgacactgtcgcaggttacaataatattttgcagcttgcacctccattaagcatcacagaggaagactttacttttatcgttaaaacaatgaaagaatgtttatcccgcattaacgggcagtaa

SEQ ID NO: 83 - protein, 449 aa, Bacillus weihenstephanensis
VQATEQTQSLKKTDEKYLWHAMRGAAPSPTNLIITKAEGAWVTDIDGNRYLDGMSGLWCVNVGYGRKELARAAFEQLEEMPYFPLTQSHVPAIKLAEKLNEWLDDEYVIFFSNSGSEANETAFKIARQYHQQKGDHGRYKFISRYRAYHGNSMGALAATGQAQRKYKYEPLGQGFLHVAPPDTYRNPEDVHTLASAEEIDRVMTWELSQTVAGVIMEPIITGGGILMPPDGYMGKVKEICEKHGALLICDEVICGFGRTGKPFGFMNYGVKPDIITMAKGITSAYLPLSATAVRREVYEAFVGSDDYDRFRHVNTFGGNPAACALALKNLEIMENEKLIERSKELGERLLYELEDVKEHPNVGDVRGKGLLLGIELVEDKQTKEPASIEKMNKVINACKEKGLIIGKNGDTVAGYNNILQLAPPLSITEEDFTFIVKTMKECLSRINGQ

SEQ ID NO: 84 - DNA, 1350 bp, Artificial
  B. weihenstephanensis KBAB4 aminotransferase codon-optimised gene
atgcaggctaccgaacaaacccaatctctgaaaaagactgacgaaaaatatctgtggcacgcgatgcgcggtgcagctccgtctccgaccaacctgattattaccaaagctgaaggcgcgtgggtgaccgacattgacggtaaccgttatctggatggcatgagcggcctgtggtgtgttaatgtcggttatggccgtaaggagctggcgcgcgcggcatttgaacaactggaagaaatgccgtacttcccgctgactcaaagccatgtgccggctatcaaactggcggaaaaactgaacgaatggctggacgacgaatacgtgattttcttctctaattctggctccgaagcaaacgaaaccgcattcaaaatcgcccgtcaatatcaccagcagaaaggtgaccacggccgctataaattcatcagccgttatcgtgcataccatggtaattctatgggtgcgctggctgctaccggtcaggctcagcgcaaatacaagtacgaaccgctgggtcagggttttctgcacgttgcaccaccggatacctaccgtaacccggaagacgtccacaccctggcttctgccgaagaaatcgatcgtgttatgacctgggagctgtcccagactgttgcgggtgttatcatggaacctattattaccggtggtggcattctgatgccgccggacggttatatgggtaaagtcaaggaaatctgcgaaaaacacggcgcgctgctgatctgcgatgaagttatctgtggcttcggtcgcaccggcaaaccatttggcttcatgaattatggcgtaaaacctgacattattaccatggctaaaggcattacttccgcttatctgccgctgagcgcgaccgcagttcgccgcgaagtttatgaagcgtttgttggttctgatgattacgaccgtttccgtcatgtaaacacgtttggcggtaacccagcggcatgtgcgctggcgctgaaaaacctggaaatcatggaaaacgaaaagctgatcgaacgtagcaaagaactgggtgaacgtctgctgtacgaactggaagatgtcaaagaacacccgaacgtgggcgatgttcgcggtaaaggcctgctgctgggtattgaactggttgaagacaaacagaccaaggaaccggcttccattgaaaagatgaacaaagtgattaacgcgtgcaaagagaaaggcctgatcattggtaagaacggtgataccgtggcaggttataacaacattctgcagctggcgccgcctctgagcatcactgaagaagatttcaccttcatcgtcaaaactatgaaggagtgcctgagccgcatcaatggtcagtaa

SEQ ID NO: 85 - DNA, 1371 bp, Pseudomonas aeruginosa
  CDS (1)..(1371)
atgaacagccaaatcaccaacgccaagacccgtgagtggcaggcgttgagccgcgaccaccatctgccgccgttcaccgactacaagcagttgaacgagaagggcgcgcggatcatcaccaaggccgaaggcgtctatatctgggacagcgagggcaacaagatcctcgatgcgatggccggcctctggtgcgtcaacgtcggctacggccgcgaggagctggtccaggccgccacccggcagatgcgcgagttgccgttctacaacctgttcttccagaccgcccacccgccggtggtcgagctggccaaggcgatcgccgacgtcgctccggaaggcatgaaccacgtgttcttcaccggctccggctccgaggccaacgacaccgtgctgcgtatggtccgccactattgggcgaccaagggccagccgcagaagaaagtggtgatcggccgctggaacggctaccacggctccaccgtcgccggcgtcagcctgggcggcatgaaggcgttgcatgagcagggtgatttccccatcccgggcatcgtccacatcgcccagccctactggtacggcgagggcggcgacatgtcgccggacgagttcggcgtctgggccgccgagcagttggagaagaagattctcgaagtgggcgaggaaaacgtcgccgccttcatcgccgagccgatccagggcgccggcggcgtgatcgtcccgccggacacctactggccgaagatccgcgagatcctcgccaagtacgacatcctgttcatcgccgacgaagtgatctgcggcttcggccgtaccggcgagtggttcggcagccagtactacggcaacgccccggacctgatgccgatcgccaagggcctcacctccggctacatccccatgggcggggtggtggtgcgcgacgagatcgtcgaagtgctcaaccagggcggcgagttctaccacggcttcacctattccggtcacccggtggcggccgccgtggccctggagaacatccgcatcctgcgcgaagagaagatcatcgagaaggtgaaggcggaaacggcaccgtatttgcagaaacgctggcaggagctggccgaccacccgttggtgggcgaagcgcgcggggtcggcatggtcgccgccctggagctggtcaagaacaagaagacccgcgagcgtttcaccgacaagggcgtcgggatgctgtgccgggaacattgtttccgcaacggtttgatcatgcgcgcggtgggcgacactatgattatctcgccgccgctggtgatcgatccgtcgcagatcgatgagttgatcaccctggcgcgcaagtgcctcgatcagaccgccgccgccgtcctggcttga

SEQ ID NO: 86 - protein, 456 aa, Pseudomonas aeruginosa
MNSQITNAKTREWQALSRDHHLPPFTDYKQLNEKGARIITKAEGVYIWDSEGNKILDAMAGLWCVNVGYGREELVQAATRQMRELPFYNLFFQTAHPPVVELAKAIADVAPEGMNHVFFTGSGSEANDTVLRMVRHYWATKGQPQKKVVIGRWNGYHGSTVAGVSLGGMKALHEQGDFPIPGIVHIAQPYWYGEGGDMSPDEFGVWAAEQLEKKILEVGEENVAAFIAEPIQGAGGVIVPPDTYWPKIREILAKYDILFIADEVICGFGRTGEWFGSQYYGNAPDLMPIAKGLTSGYIPMGGVVVRDEIVEVLNQGGEFYHGFTYSGHPVAAAVALENIRILREEKIIEKVKAETAPYLQKRWQELADHPLVGEARGVGMVAALELVKNKKTRERFTDKGVGMLCREHCFRNGLIMRAVGDTMIISPPLVIDPSQIDELITLARKCLDQTAAAVLA

SEQ ID NO: 87 - DNA, 70 bp, Artificial
  primer
ggggacaagtttgtacaaaaaagcaggctaggaggaattaaccatgaacagccaaatcaccaacgccaag

SEQ ID NO: 88 - DNA, 49 bp, Artificial
  primer
ggggaccactttgtacaagaaagctgggttcaagccaggacggcggcgg

SEQ ID NO: 89 - DNA, 849 bp, Bacillus subtilis
  CDS (1)..(843)
atgaaggttttagtcaatggccggctgattgggcgcagtgaagcatcaatcgatttggaagatcgcggttatcagtttggtgacggcatctatgaagtgatcagggtgtacaaaggagtattgttcggcttacgtgagcatgcagagcgttttttcagaagtgctgctgaaatcggaatttcactgccattcagtatagaagatctcgagtgggacctgcaaaagcttgtacaggaaaatgcggtcagtgagggagcggtatacattcagacaacaagaggtgtggccccgcgaaaacaccagtatgaagccggcctcgagccgcagactactgcctatacgtttacggtgaaaaaaccggagcaagagcaggcatacggagtggcggccattacagatgaggatcttcgctggttaagatgtgatatcaaaagtctgaatttactgtataatgtcatgacgaagcaaagggcctatgaagccggagcatttgaagccattttacttagggacggcgttgttacggagggtacatcctctaacgtttatgccgttatcaacggcacagtgcgaacacatccggctaatcggctcattctcaatggaattacacggatgaatattttaggactgattgagaagaatgggatcaaactggatgagactcctgtcagtgaagaagagttgaaacaggcggaagagatctttatttcgtcaacgacggcagaaattattccggtcgtgacgctcgatggacaatcgatcggaagcgggaaacccggaccggtgaccaaacagcttcaggctgcttttcaagaaagcattcaacaggctgctagcatttcataa

SEQ ID NO: 90 - protein, 281 aa, Bacillus subtilis
MKVLVNGRLIGRSEASIDLEDRGYQFGDGIYEVIRVYKGVLFGLREHAERFFRSAAEIGISLPFSIEDLEWDLQKLVQENAVSEGAVYIQTTRGVAPRKHQYEAGLEPQTTAYTFTVKKPEQEQAYGVAAITDEDLRWLRCDIKSLNLLYNVMTKQRAYEAGAFEAILLRDGVVTEGTSSNVYAVINGTVRTHPANRLILNGITRMNILGLIEKNGIKLDETPVSEEELKQAEEIFISSTTAEIIPVVTLDGQSIGSGKPGPVTKQLQAAFQESIQQAASI

SEQ ID NO: 91 - DNA, 1347 bp, Bacillus subtilis
  CDS (1)..(1347)
atgactcatgatttgatagaaaaaagtaaaaagcacctctggctgccatttacccaaatgaaagattatgatgaaaaccccttaatcatcgaaagcgggactggaatcaaagtcaaagacataaacggcaaggaatactatgacggtttttcatcggtttggcttaatgtccacggacaccgcaaaaaagaactagatgacgccataaaaaaacagctcggaaaaattgcgcactccacgttattgggcatgaccaatgttccagcaacccagcttgccgaaacattaatcgacatcagcccaaaaaagctcacgcgggtcttttattcagacagcggcgcagaggcgatggaaatagccctaaaaatggcgtttcagtattggaagaacatcgggaagcccgagaaacaaaaattcatcgcaatgaaaaacgggtatcacggtgatacgattggcgccgtcagtgtcggttcaattgagctttttcaccacgtatacggcccgttgatgttcgagagttacaaggccccgattccttatgtgtatcgttctgaaagcggtgatcctgatgagtgccgtgatcagtgcctccgagagcttgcacagctgcttgaggaacatcatgaggaaattgccgcgctttccattgaatcaatggtacaaggcgcgtccggtatgatcgtgatgccggaaggatatttggcaggcgtgcgcgagctatgtacaacatacgatgtcttaatgatcgttgatgaagtcgctacaggctttggccgtacaggaaaaatgtttgcgtgcgagcacgagaatgtccagcctgatctgatggctgccggtaaaggcattacaggaggctatttgccaattgccgttacgtttgccactgaagacatctataaggcattctatgatgattatgaaaacctaaaaacctttttccatggccattcctatacaggcaatcagcttggctgtgcggttgcgcttgaaaatctggcattatttgaatctgaaaacattgtggaacaagtagcggaaaaaagtaaaaagctccattttcttcttcaagatctgcacgctcttcctcatgttggggatattcggcagcttggctttatgtgcggtgcagagcttgtacgatcaaaggaaactaaagaaccttacccggctgatcggcggattggatacaaagtttccttaaaaatgagagagttaggaatgctgacaagaccgcttggggacgtgattgcatttcttcctcctcttgccagcacagctgaagagctctcggaaatggttgccattatgaaacaagcgatccacgaggttacgagccttgaagattga

SEQ ID NO: 92 - protein, 448 aa, Bacillus subtilis
MTHDLIEKSKKHLWLPFTQMKDYDENPLIIESGTGIKVKDINGKEYYDGFSSVWLNVHGHRKKELDDAIKKQLGKIAHSTLLGMTNVPATQLAETLIDISPKKLTRVFYSDSGAEAMEIALKMAFQYWKNIGKPEKQKFIAMKNGYHGDTIGAVSVGSIELFHHVYGPLMFESYKAPIPYVYRSESGDPDECRDQCLRELAQLLEEHHEEIAALSIESMVQGASGMIVMPEGYLAGVRELCTTYDVLMIVDEVATGFGRTGKMFACEHENVQPDLMAAGKGITGGYLPIAVTFATEDIYKAFYDDYENLKTFFHGHSYTGNQLGCAVALENLALFESENIVEQVAEKSKKLHFLLQDLHALPHVGDIRQLGFMCGAELVRSKETKEPYPADRRIGYKVSLKMRELGMLTRPLGDVIAFLPPLASTAEELSEMVAIMKQAIHEVTSLED

SEQ ID NO: 93 - DNA, 1467 bp, Rhodobacter sphaeroides
  CDS (1)..(1467)
atgcccggttgcgggggcttgcccgggaatgaaccgaaatgcggacgagaggggaggtcggcgatgacgcggaatgacgcgacgaatgctgccggagcggtgggcgcggcgatgcgggatcacatcctcttgcctgcacaggaaatggcgaagctcggcaagtccgcgcagccggtgctgactcatgccgagggcatctatgtccataccgaggacggccgccgcctgatcgacgggccggcgggcatgtggtgcgcgcaggtgggctacggccgccgcgagatcgtcgatgccatggcgcatcaggcgatggtgctgccctatgcctcgccctggtatatggccacgagccccgcggcgcggctggcggagaagatcgccacgctgacgccgggcgatctcaaccggatctttttcaccacgggcgggtcgaccgcggtggacagcgcgctgcgcttctcggaattctacaacaacgtgctgggccggccgcagaagaagcgcatcatcgtgcgctacgacggctatcacggctcgacggcgctcaccgccgcctgcaccggccgcaccggcaactggccgaacttcgacatcgcgcaggaccggatctcgttcctctcgagccccaatccgcgccacgccggcaaccgcagccaggaggcgttcctcgacgatctggtgcaggaattcgaggaccggatcgagagcctcggccccgacacgatcgcggccttcctggccgagccgatcctcgcctcgggcggcgtcattattccgcccgcaggctatcatgcgcgcttcaaggcgatctgcgagaagcacgacatcctctatatctcggacgaggtggtgacgggcttcggccgttgcggcgagtggttcgcctcggagaaggtgttcggggtggtgccggacatcatcaccttcgccaagggcgtgacctcgggctatgtgccgctcggcggccttgcgatctccgaggcggtgctggcgcggatctcgggcgagaatgccaagggaagctggttcaccaacggctatacctacagcaatcagccggtggcctgcgccgcggcgcttgccaacatcgagctgatggagcgcgagggcatcgtcgatcaggcgcgcgagatggcggactatttcgccgcggcgctggcttcgctgcgcgatctgccgggcgtggcggaaacccggtcggtgggcctcgtgggttgcgtgcaatgcctgctcgacccgacccgggcggacggcacggccgaggacaaggccttcaccctgaagatcgacgagcgctgcttcgagctcgggctgatcgtgcgcccgctgggcgatctctgcgtgatctcgccgccgctcatcatctcgcgcgcgcagatcgacgagatggtcgcgatcatgcggcaggccatcaccgaagtgagcgccgcccacggtctgaccgcgaaagaaccggccgccgtctga

SEQ ID NO: 94 - protein, 488 aa, Rhodobacter sphaeroides
MPGCGGLPGNEPKCGREGRSAMTRNDATNAAGAVGAAMRDHILLPAQEMAKLGKSAQPVLTHAEGIYVHTEDGRRLIDGPAGMWCAQVGYGRREIVDAMAHQAMVLPYASPWYMATSPAARLAEKIATLTPGDLNRIFFTTGGSTAVDSALRFSEFYNNVLGRPQKKRIIVRYDGYHGSTALTAACTGRTGNWPNFDIAQDRISFLSSPNPRHAGNRSQEAFLDDLVQEFEDRIESLGPDTIAAFLAEPILASGGVIIPPAGYHARFKAICEKHDILYISDEVVTGFGRCGEWFASEKVFGVVPDIITFAKGVTSGYVPLGGLAISEAVLARISGENAKGSWFTNGYTYSNQPVACAAALANIELMEREGIVDQAREMADYFAAALASLRDLPGVAETRSVGLVGCVQCLLDPTRADGTAEDKAFTLKIDERCFELGLIVRPLGDLCVISPPLIISRAQIDEMVAIMRQAITEVSAAHGLTAKEPAAV

SEQ ID NO: 95 - DNA, 837 bp, Legionella pneumophila
  CDS (1)..(837)
atgagtatcgcatttgttaacggcaagtattgttgtcaatctgaagcaaaaatttcaatatttgatcgagggtttctttttggtgactcggtttatgaagtgctgcctgtttaccatgggcagccttactttgtagaccaacatcttgaccgattattctcaaatatgaaaaaaattaagatgattataccaaattatgattggcatggtttaattcatagactaatatcagaaaataatggcggtaatttacaagtatatatccaagtcacacgagggaatcaaggggtgcgcaagcatgatatccctacttccatcacaccttctgttatcgcattcactatgcataatccatttcccaccctcgaagataaggaacagggaatgtcagcaaaactggttgaagattttcggtggatgagatgtgatataaaaactacttctttaattgccaatatattactgaatgatgaggctgtatctgcaggattccacactgcaattcttgcccggaacggtctaattacagagggaagtagtaccaacgtatttattgtcgcacaggatggtgttattaagacaccacccatgaataatttctgtttaccaggaattactcggcaagttgttattgaaataattaaaaaattagatttaaagttcagagaaatagaaattagcatttcagagcttttttctgctcaggaagtttggataacaagtacgacaaaagaagtattccctattacaaagattaatgactctttgattaatggcggaaaagttggcgaatattggcggataattaatgattcctaccaacaactagtaaactaa

SEQ ID NO: 96 - protein, 278 aa, Legionella pneumophila
MSIAFVNGKYCCQSEAKISIFDRGFLFGDSVYEVLPVYHGQPYFVDQHLDRLFSNMKKIKMIIPNYDWHGLIHRLISENNGGNLQVYIQVTRGNQGVRKHDIPTSITPSVIAFTMHNPFPTLEDKEQGMSAKLVEDFRWMRCDIKTTSLIANILLNDEAVSAGFHTAILARNGLITEGSSTNVFIVAQDGVIKTPPMNNFCLPGITRQVVIEIIKKLDLKFREIEISISELFSAQEVWITSTTKEVFPITKINDSLINGGKVGEYWRIINDSYQQLVN

SEQ ID NO: 97 - DNA, 861 bp, Nitrosomonas europaea
  CDS (1)..(861)
atgatttacctcaatggcaaatttctgccgatggaacaggctaccgttccagtgctggatagaggcttcatcttcggtgatggtgtctatgaagtcataccggtttattcacgtaaaccgttccggctgggcgaacatctttcccggctgcagcacagtctggatggcatacgtctccagaatccgcacactgaagaacaatgggctggtctgatcgaacgcatcatcgagctgaatgaaggtgatgatcagtacctttacctgcacattacacgcggggtggcaaaacgtgaccatgcctttcctcgcgaagtaacgcccactgtcttcatcatgagcaacccgcttccggctccacctgcaaaattgctcgtttccggagtttcagcgattaccgccagggataatcgctgggggcgctgtgatatcaaagccatttcactgttgccaaatatcttattgcgccagcttgccgtggacgcacaagccatggaaacgatcctgttacgcgatggtctgttgaccgaaggggccgccagcaatattttcatcgtaaaagacgacctgctgctgaccccccccaaagatcaccgtatattgcctggcattacttatgatgtagtactggaactggctgaaacacatggtgttccacatgcgacaagagaaatatcagagcttgagttacgtactgcacgggaaatcatgctgacttcttccaccaaagaaattctcccgatcacacagctggatggacaaccgatcggtaatggcaccccagggccagtatttcagcaactggatcggctctatcaggcatataagctggaagtcatgcgcgggcatgctccacgccagtaa

SEQ ID NO: 98 - protein, 286 aa, Nitrosomonas europaea
MIYLNGKFLPMEQATVPVLDRGFIFGDGVYEVIPVYSRKPFRLGEHLSRLQHSLDGIRLQNPHTEEQWAGLIERIIELNEGDDQYLYLHITRGVAKRDHAFPREVTPTVFIMSNPLPAPPAKLLVSGVSAITARDNRWGRCDIKAISLLPNILLRQLAVDAQAMETILLRDGLLTEGAASNIFIVKDDLLLTPPKDHRILPGITYDVVLELAETHGVPHATREISELELRTAREIMLTSSTKEILPITQLDGQPIGNGTPGPVFQQLDRLYQAYKLEVMRGHAPRQ

SEQ ID NO: 99 - DNA, 1293 bp, Neisseria gonorrhoeae
  CDS (1)..(1293)
atgaggataaatatgaaccgtaacgaaattttattcgaccgcgccaaggccatcatccccggcggcgtgaattcgcccgtgcgcgcattcggcagcgtcggcggcgtgccgcgcttcatcaaaaaagccgaaggcgcgtatgtttgggacgaaaacggcacgcgctacaccgattatgtcggctcttgggggcctgcgattgtcggacacgcgcatcccgaagtcgtcgaagccgtgcgcgaagctgcgttgggcggtttgtcgttcggcgcgcccaccgaaggcgaaatcgccattgccgaacaaattgccgaaattatgccgtctgtcgaacggctgcgcctcgtcagctccggcacggaagcgacgatgactgccatccgtctggcacgcggttttaccggccgcgacaaaatcatcaaatttgaaggctgctaccacggccattccgacagcctgttggtgaaagcaggcagcggtctgcttaccttcggcaatccttcttccgccggtgtgcctgccgactttaccaaacatactttggtactcgaatacaacaacatcgcccaactcgaagaagcctttgcccaaagcggcgacgaaatcgcctgcgtgattgtcgaacccttcgtcggcaatatgaacctcgtccgcccgaccgaagcctttgtcaaagccttgcgcggattgaccgaaaaacacggcgcggtgttgatttacgacgaagtgatgaccggtttccgcgtcgcgctcggcggcgcgcagtcgctgcacggcatcacgcccgacctgaccacgatgggcaaagtcatcggcggcggtatgccgcttgccgcgttcggcggacgcaaagacatcatggaatgtatttccccgttgggcggcgtgtatcaggcaggtacattatcaggcaacccgattgccgtcgccgccggcttgaaaacgctggaaatcatccagcgcgaaggcttctatgaaaacctgaccgccttgacacaacgccttgccaacggtattgccgccgccaaagcgcacggtatcgagtttgccgccgacagcgtgggcggtatgttcggtctgtatttcgccgcacacgtgccgcgaaactatgccgatatggcgcgctccaatatcgacgctttcaaacgcttcttccacggcatgctcgaccgcggcattgccttcggcccgtccgcttatgaagcgggtttcgtttccgccgcgcatacgcccgagctgattgacgaaacggttgcggttgcggttgaagtgttcaaggcgatggctgcatga

SEQ ID NO: 100 - protein, 430 aa, Neisseria gonorrhoeae
MRINMNRNEILFDRAKAIIPGGVNSPVRAFGSVGGVPRFIKKAEGAYVWDENGTRYTDYVGSWGPAIVGHAHPEVVEAVREAALGGLSFGAPTEGEIAIAEQIAEIMPSVERLRLVSSGTEATMTAIRLARGFTGRDKIIKFEGCYHGHSDSLLVKAGSGLLTFGNPSSAGVPADFTKHTLVLEYNNIAQLEEAFAQSGDEIACVIVEPFVGNMNLVRPTEAFVKALRGLTEKHGAVLIYDEVMTGFRVALGGAQSLHGITPDLTTMGKVIGGGMPLAAFGGRKDIMECISPLGGVYQAGTLSGNPIAVAAGLKTLEIIQREGFYENLTALTQRLANGIAAAKAHGIEFAADSVGGMFGLYFAAHVPRNYADMARSNIDAFKRFFHGMLDRGIAFGPSAYEAGFVSAAHTPELIDETVAVAVEVFKAMAA

SEQ ID NO: 101 - DNA, 924 bp, Pseudomonas aeruginosa
  CDS (1)..(924)
atgtcgatggccgatcgtgatggcgtgatctggtatgacggtgaactggtgcagtggcgcgacgcgaccacgcacgtgctgacccataccctgcactatggaatgggcgtgttcgagggcgtgcgcgcctacgacaccccgcagggcacggcgatcttccgcctgcaggcgcataccgaccggctgttcgactccgcgcacatcatgaacatgcagatcccgtacagccgcgacgagatcaacgaggcgacccgcgccgccgtgcgcgagaacaacctggaaagcgcctatatccgcccgatggtgttctacggaagcgaaggcatgggcctgcgcgccagcggcctgaaggtccatgtgatcatcgccgcctggagctggggcgcctacatgggcgaggaagccctgcagcaaggcatcaaggtgcgcaccagttccttcacccgccaccacgtcaacatctcgatgacccgcgccaagtccaacggcgcctacatcaactcgatgctggccctccaggaagcgatctccggcggcgccgacgaggccatgatgctcgatccggaaggctacgtggccgaaggctccggcgagaacatcttcatcatcaaggatggcgtgatctacaccccggaagtcaccgcctgcctgaacggcatcactcgtaacactatcctgaccctggccgccgaacacggttttaaactggtcgagaagcgcatcacccgcgacgaggtgtacatcgccgacgaggccttcttcactggcactgccgcggaagtcacgccgatccgcgaagtggacggtcgcaagatcggcgccggccgccgtggcccggtcaccgaaaagctgcagaaagcctatttcgacctggtcagcggcaagaccgaggcccacgccgagtggcgtaccctggtcaagtaa

SEQ ID NO: 102 - protein, 307 aa, Pseudomonas aeruginosa
MSMADRDGVIWYDGELVQWRDATTHVLTHTLHYGMGVFEGVRAYDTPQGTAIFRLQAHTDRLFDSAHIMNMQIPYSRDEINEATRAAVRENNLESAYIRPMVFYGSEGMGLRASGLKVHVIIAAWSWGAYMGEEALQQGIKVRTSSFTRHHVNISMTRAKSNGAYINSMLALQEAISGGADEAMMLDPEGYVAEGSGENIFIIKDGVIYTPEVTACLNGITRNTILTLAAEHGFKLVEKRITRDEVYIADEAFFTGTAAEVTPIREVDGRKIGAGRRGPVTEKLQKAYFDLVSGKTEAHAEWRTLVK

SEQ ID NO: 103 - DNA, 1407 bp, Rhodopseudomonas palustris
  CDS (1)..(1407)
atgaagctgataccgtgccgcgcctttcaccccccggccgcgcagtgcatgaggagcgccatgttagacaagatcaagcccacgtccgccgtcaacgcgccgaacgatctcaacgcgttctggatgccgttcaccgcgaaccgggccttcaagcgcgcgccgaagatggtcgtgggtgccgaaggcatgcactacatcaccgccgatggtcgcaagatcatcgacgccgcctcgggcatgtggtgcaccaatgcgggccatggccgcaaggaaatcgccgaggcgatcaaggcgcaggccgatgaactcgacttctcgccgccgttccagttcggccagccgaaggcgttcgaactcgccagccggatcgccgatctggcgccggaaggcctcgatcacgtgttcttctgcaattcgggctcggaagccggcgacaccgcgctgaagatcgcggtcgcctatcagcagatcaagggccagggctcacgcacccgcctgatcggccgcgagcgcggctatcacggcgtcggcttcggcggcaccgcggtcggcggcatcggcaacaaccgcaagatgttcggtccgctgctcaacggcgtcgatcatctgcctgcgacttatgatcgcgacaagcaggctttcaccatcggcgagccggaatacggcgcgcacttcgccgaagcgcttgaaggcctcgtcaatctgcacggcgccaacaccatcgcggcggtgatcgtcgagccgatggccggctccaccggcgtgctgccggcgccgaagggctatctcaagaagctgcgcgagatcaccaagaagcacggcatcctgctgatcttcgacgaggtcatcaccggctacggccgtctcggctatgccttcgcgtccgaacgttacggcgtcaccccggacatgatcaccttcgccaagggcgtcaccaatggtgcggtgccgatgggcggcgtgatcacctcggcggagatccacgatgcgttcatgaccggccccgagcacgcggtcgagctggcgcacggctacacctattcggcgcatccgctcgcctgcgcggccggcatcgccaccctcgacatctaccgcgacgagaagctgttcgagcgcgccaaggcgctggagccgaagtttgccgaggcggtgatgtcgctgaagtcggccccgaacgtggtcgacatccgcaccgtcggcctgacggcgggtatcgacctcgcttcgatcgccgatgcggtcggcaagcgtggcttcgaagcgatgaatgccggcttccacgaccacgagctgatgctgcggatcgccggcgacaccctggcgctgaccccgccgctgatcctcagcgaggaccacatcggtgagatcgtcgacaaggtcggcaaggtgatccgcgcggtcgcctga

SEQ ID NO: 104 - protein, 468 aa, Rhodopseudomonas palustris
MKLIPCRAFHPPAAQCMRSAMLDKIKPTSAVNAPNDLNAFWMPFTANRAFKRAPKMVVGAEGMHYITADGRKIIDAASGMWCTNAGHGRKEIAEAIKAQADELDFSPPFQFGQPKAFELASRIADLAPEGLDHVFFCNSGSEAGDTALKIAVAYQQIKGQGSRTRLIGRERGYHGVGFGGTAVGGIGNNRKMFGPLLNGVDHLPATYDRDKQAFTIGEPEYGAHFAEALEGLVNLHGANTIAAVIVEPMAGSTGVLPAPKGYLKKLREITKKHGILLIFDEVITGYGRLGYAFASERYGVTPDMITFAKGVTNGAVPMGGVITSAEIHDAFMTGPEHAVELAHGYTYSAHPLACAAGIATLDIYRDEKLFERAKALEPKFAEAVMSLKSAPNVVDIRTVGLTAGIDLASIADAVGKRGFEAMNAGFHDHELMLRIAGDTLALTPPLILSEDHIGEIVDKVGKVIRAVA

SEQ ID NO: 105 - DNA, 1263 bp, Escherichia coli
  CDS (1)..(1263)
atgccacattcactgttcagcaccgataccgatctcaccgccgaaaatctgctgcgtttgcccgctgaatttggctgcccggtgtgggtctacgatgcgcaaattattcgtcggcagattgcagcgctgaaacagtttgatgtggtgcgctttgcacagaaagcctgttccaatattcatattttgcgcttaatgcgtgagcagggcgtgaaagtggattccgtctcgttaggcgaaatagagcgtgcgttggcggcgggttacaatccgcaaacgcaccccgatgatattgtttttacggcagatgttatcgatcaggcgacgcttgaacgcgtcagtgaattgcaaattccggtgaatgcgggttctgttgatatgctcgaccaactgggccaggtttcgccagggcatcgggtatggctgcgcgttaatccggggtttggtcacggacatagccaaaaaaccaataccggtggcgaaaacagcaagcacggtatctggtacaccgatctgcccgccgcactggacgtgatacaacgtcatcatctgcagctggtcggcattcacatgcacattggttctggcgttgattatgcccatctggaacaggtgtgtggtgctatggtgcgtcaggtcatcgaattcggtcaggatttacaggctatttctgcgggcggtgggctttctgttccttatcaacagggtgaagaggcggttgataccgaacattattatggtctgtggaatgccgcgcgtgagcaaatcgcccgccatttgggccaccctgtgaaactggaaattgaaccgggtcgcttcctggtagcgcagtctggcgtattaattactcaggtgcggagcgtcaaacaaatggggagccgccactttgtgctggttgatgccgggttcaacgatctgatgcgcccggcaatgtacggtagttaccaccatatcagtgccctggcagctgatggtcgttctctggaacacgcgccaacggtggaaaccgtcgtcgccggaccgttatgtgaatcgggcgatgtctttacccagcaggaagggggaaatgttgaaacccgcgccttgccggaagtgaaggcaggtgattatctggtactgcatgatacaggggcatatggcgcatcaatgtcatccaactacaatagccgtccgctgttaccagaagttctgtttgataatggtcaggcgcggttgattcgccgtcgccagaccatcgaagaattactggcgctggaattgctttaa

SEQ ID NO: 106 - protein, 420 aa, Escherichia coli
MPHSLFSTDTDLTAENLLRLPAEFGCPVWVYDAQIIRRQIAALKQFDVVRFAQKACSNIHILRLMREQGVKVDSVSLGEIERALAAGYNPQTHPDDIVFTADVIDQATLERVSELQIPVNAGSVDMLDQLGQVSPGHRVWLRVNPGFGHGHSQKTNTGGENSKHGIWYTDLPAALDVIQRHHLQLVGIHMHIGSGVDYAHLEQVCGAMVRQVIEFGQDLQAISAGGGLSVPYQQGEEAVDTEHYYGLWNAAREQIARHLGHPVKLEIEPGRFLVAQSGVLITQVRSVKQMGSRHFVLVDAGFNDLMRPAMYGSYHHISALAADGRSLEHAPTVETVVAGPLCESGDVFTQQEGGNVETRALPEVKAGDYLVLHDTGAYGASMSSNYNSRPLLPEVLFDNGQARLIRRRQTIEELLALELL

SEQ ID NO: 107 - DNA, 1265 bp, Artificial
  Escherichia.coli diaminopimelate decarboxylase LysA codon optimised gene
atatgccacactctctgttttctactgatactgatctgactgcggaaaacctgctgcgtctgccggctgaattcggttgtccggtatgggtgtacgacgctcagattattcgtcgccagatcgcagcactgaagcagttcgatgtagtgcgttttgcacagaaggcgtgctccaacatccatatcctgcgcctgatgcgtgagcagggcgttaaagttgactccgtctctctgggtgagattgagcgcgccctggcagccggctataacccacagacccatcctgacgacattgtatttactgccgacgtgatcgaccaggctactctggaacgcgtttctgaactgcagatcccggttaatgctggttctgtggacatgctggaccagctgggccaggtatccccaggtcatcgtgtgtggctgcgtgtcaacccaggtttcggccacggccactctcagaaaactaacactggtggtgagaactccaagcatggcatttggtataccgatctgccggctgcactggacgtaatccagcgtcaccacctgcagctggtgggcatccacatgcacattggctccggcgtagactacgcccacctggagcaagtctgcggtgctatggtacgtcaggtaatcgagttcggccaagatctgcaggcaatcagcgctggtggcggcctgtctgtaccttatcagcagggcgaggaggcggttgacactgagcactactacggtctgtggaacgccgctcgtgagcaaattgcacgtcacctgggccacccggtgaaactggagatcgagccgggccgcttcctggtagcacagtccggcgtactgattacccaggtacgctctgttaaacagatgggctcccgtcactttgtgctggtagacgcaggcttcaacgacctgatgcgtccggctatgtatggttcctatcatcacatctctgcgctggccgccgacggccgctctctggaacacgcgccgacggttgaaacggtggtggctggtccgctgtgcgagtccggcgacgttttcactcagcaggagggcggcaatgtagagacgcgtgcgctgccggaagtgaaagccggtgattatctggtgctgcatgataccggcgcctatggtgcgagcatgagcagcaactacaactctcgcccgctgctgccggaggtcctgttcgataacggccaagcccgcctgatccgtcgtcgtcagaccatcgaggaactgctggcactggagctgctgtaa

SEQ ID NO: 108 - DNA, 1692 bp, Saccharomyces cerevisiae
  CDS (1)..(1692)
atgtctgaaattactttgggtaaatatttgttcgaaagattaaagcaagtcaacgttaacaccgttttcggtttgccaggtgacttcaacttgtccttgttggacaagatctacgaagttgaaggtatgagatgggctggtaacgccaacgaattgaacgctgcttacgccgctgatggttacgctcgtatcaagggtatgtcttgtatcatcaccaccttcggtgtcggtgaattgtctgctttgaacggtattgccggttcttacgctgaacacgtcggtgttttgcacgttgttggtgtcccatccatctctgctcaagctaagcaattgttgttgcaccacaccttgggtaacggtgacttcactgttttccacagaatgtctgccaacatttctgaaaccactgctatgatcactgacattgctaccgccccagctgaaattgacagatgtatcagaaccacttacgtcacccaaagaccagtctacttaggtttgccagctaacttggtcgacttgaacgtcccagctaagttgttgcaaactccaattgacatgtctttgaagccaaacgatgctgaatccgaaaaggaagtcattgacaccatcttggctttggtcaaggatgctaagaacccagttatcttggctgatgcttgttgttccagacacgacgtcaaggctgaaactaagaagttgattgacttgactcaattcccagctttcgtcaccccaatgggtaagggttccattgacgaacaacacccaagatacggtggtgtttacgtcggtaccttgtccaagccagaagttaaggaagccgttgaatctgctgacttgattttgtctgtcggtgctttgttgtctgatttcaacaccggttctttctcttactcttacaagaccaagaacattgtcgaattccactccgaccacatgaagatcagaaacgccactttcccaggtgtccaaatgaaattcgttttgcaaaagttgttgaccactattgctgacgccgctaagggttacaagccagttgctgtcccagctagaactccagctaacgctgctgtcccagcttctaccccattgaagcaagaatggatgtggaaccaattgggtaacttcttgcaagaaggtgatgttgtcattgctgaaaccggtacctccgctttcggtatcaaccaaaccactttcccaaacaacacctacggtatctctcaagtcttatggggttccattggtttcaccactggtgctaccttgggtgctgctttcgctgctgaagaaattgatccaaagaagagagttatcttattcattggtgacggttctttgcaattgactgttcaagaaatctccaccatgatcagatggggcttgaagccatacttgttcgtcttgaacaacgatggttacaccattgaaaagttgattcacggtccaaaggctcaatacaacgaaattcaaggttgggaccacctatccttgttgccaactttcggtgctaaggactatgaaacccacagagtcgctaccaccggtgaatgggacaagttgacccaagacaagtctttcaacgacaactctaagatcagaatgattgaaatcatgttgccagtcttcgatgctccacaaaacttggttgaacaagctaagttgactgctgctaccaacgctaagcaataa

SEQ ID NO: 109 - protein, 563 aa, Saccharomyces cerevisiae
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 110 - DNA, 1692 bp, Artificial
  Saccharomyces cerevisiae pyruvate decarboxylase Pdc codon optimised gene
atgtccgagatcactctgggcaaatacctgtttgaacgtctgaaacaggtgaacgttaataccgtattcggcctgccgggtgatttcaacctgtccctgctggacaaaatctatgaagttgaaggtatgcgttgggctggcaacgctaacgagctgaacgcagcgtacgcggcagatggttacgctcgtatcaaaggtatgtcttgtatcatcaccaccttcggtgttggtgagctgagcgccctgaacggcatcgccggctcctatgcagagcacgtgggcgtgctgcacgttgtgggtgtaccgtccatcagcgcccaggcaaaacagctgctgctgcaccacaccctgggtaacggcgactttaccgttttccatcgtatgtctgcgaacatcagcgaaactactgcaatgattactgacatcgctacggcaccggcagaaatcgaccgttgcattcgtaccacgtacgttactcagcgcccggtttatctgggcctgccagccaacctggtggatctgaacgtcccggctaaactgctgcagactccgatcgatatgtctctgaaacctaacgacgcagaatctgagaaagaagttatcgatactattctggctctggtgaaagatgcaaagaacccagttatcctggctgacgcatgttgctctcgtcatgatgtaaaggcagaaaccaaaaagctgatcgacctgacgcagttcccggcgttcgttaccccgatgggcaagggttccatcgatgagcagcacccgcgttatggtggtgtatacgttggcacgctgtccaaaccggaggtaaaagaagcggttgaaagcgcagatctgatcctgtctgttggtgcactgctgagcgacttcaacaccggttctttctcctatagctacaagaccaaaaacattgtggagtttcactccgatcacatgaaaatccgcaacgcgacctttcctggtgtgcagatgaaattcgtactgcagaaactgctgaccaccatcgccgacgctgcgaaaggttataaaccggtagctgtgccggcacgtaccccggcgaacgccgcggttcctgcatccactccactgaagcaggaatggatgtggaatcagctgggtaatttcctgcaagaaggcgacgttgtaatcgcagaaaccggcactagcgcgtttggcattaaccagacgaccttcccaaacaacacctacggtatcagccaagtcctgtggggctctatcggcttcaccaccggtgcaaccctgggtgcggctttcgctgctgaggagatcgacccgaagaaacgtgttatcctgttcatcggtgacggctccctgcagctgaccgtccaggagatttctaccatgatccgctggggcctgaaaccgtacctgtttgtgctgaacaacgacggctacactattgagaaactgatccacggtccgaaagcacagtataatgagatccagggttgggatcatctgtctctgctgccgacctttggcgctaaagactacgagacccaccgcgtggctaccaccggcgagtgggataaactgacgcaggataaatccttcaatgacaatagcaagattcgtatgatcgaaatcatgctgccggtctttgatgctccgcagaacctggtagagcaagcaaaactgaccgcggcaactaacgctaaacagtaa

SEQ ID NO: 111 - DNA, 1707 bp, Zymomonas mobilis
  CDS (1)..(1707)
atgagttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgccgctgaagcgatttacaccccggaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggcacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcattcgcttccccagcgtccatctgaaagactatctgacccgtttggctcagaaagtttccaagaaaaccggtgcattggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacattggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccgccgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctag

SEQ ID NO: 112 - protein, 568 aa, Zymomonas mobilis
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGIRFPSVHLKDYLTRLAQKVSKKTGALDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTAEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 113 - DNA, 1707 bp, Artificial
  Zymomonas mobilis pyruvate decarboxylase PdcI472A codon optimised gene
atgtcttatactgttggtacttatctggctgagcgtctggtgcaaatcggcctgaaacaccactttgcagttgctggcgactacaacctggttctgctggataacctgctgctgaacaaaaacatggagcaagtttattgctgtaacgagctgaactgcggcttctctgcggagggttatgcgcgtgcgaaaggtgccgctgcagcagtcgtaacctactctgtgggcgctctgtccgcgttcgacgcaatcggtggcgcttacgctgaaaacctgccggtgatcctgattagcggtgcgccgaataataacgaccatgctgctggccacgttctgcaccacgccctgggtaaaactgattaccattaccagctggagatggctaaaaacatcactgcagcagcagaagcgatctacaccccggaagaggctccggcaaaaatcgaccacgtgattaaaaccgctctgcgtgagaaaaagccggtatacctggaaatcgcgtgcaacatcgcgtctatgccgtgcgccgcaccgggtccggcttctgccctgttcaacgatgaggcgagcgatgaggcatctctgaacgcagcagtagaagaaaccctgaaatttatcgcaaaccgtgacaaagtagcagtcctggtaggttctaaactgcgtgcggctggtgcggaagaggctgcggtaaagttcgcggatgctctgggcggtgcagtggcgaccatggcagcggctaaatccttcttcccagaggagaacccgcattacattggtacctcctggggcgaagtttcctaccctggtgtggagaaaaccatgaaagaagccgatgctgtgattgccctggcgcctgtattcaacgattattccaccaccggttggaccgatatcccggacccgaagaaactggtcctggctgaaccgcgctccgtagtagtgaatggcattcgtttcccgtccgtacacctgaaggattacctgacgcgtctggcacagaaagtatccaagaaaactggcgcgctggacttctttaaatccctgaacgctggtgagctgaaaaaggcggctccggccgatccgtccgcaccgctggtgaacgcagagattgcacgtcaggttgaggcactgctgacgccgaacaccaccgtaatcgcggaaacgggcgactcttggttcaacgcacagcgcatgaaactgccgaacggtgcccgcgttgaatatgaaatgcagtggggtcacatcggctggtctgtcccagcagcgtttggttacgcggttggtgcaccggagcgtcgcaacatcctgatggtgggtgacggctccttccagctgactgctcaggaggtggcgcagatggtgcgcctgaagctgccggttatcattttcctgatcaacaactacggctacaccgccgaggtaatgatccacgatggtccgtacaacaacatcaaaaactgggactacgccggtctgatggaggtttttaacggtaacggcggttacgacagcggtgctggtaagggtctgaaagccaaaaccggtggcgaactggcagaggcgattaaagttgcgctggcaaacaccgatggcccgaccctgatcgagtgcttcatcggccgtgaggactgcaccgaggagctggtcaaatggggcaaacgtgtggcggctgctaactctcgcaagccggtaaacaaactgctgtaa

SEQ ID NO: 114 - DNA, 1644 bp, Lactococcus lactis
  CDS (1)..(1644)
atgtatacagtaggagattacctgttagaccgattacacgagttgggaattgaagaaatttttggagttcctggtgactataacttacaatttttagatcaaattatttcacgcgaagatatgaaatggattggaaatgctaatgaattaaatgcttcttatatggctgatggttatgctcgtactaaaaaagctgccgcatttctcaccacatttggagtcggcgaattgagtgcgatcaatggactggcaggaagttatgccgaaaatttaccagtagtagaaattgttggttcaccaacttcaaaagtacaaaatgacggaaaatttgtccatcatacactagcagatggtgattttaaacactttatgaagatgcatgaacctgttacagcagcgcggactttactgacagcagaaaatgccacatatgaaattgaccgagtactttctcaattactaaaagaaagaaaaccagtctatattaacttaccagtcgatgttgctgcagcaaaagcagagaagcctgcattatctttagaaaaagaaagctctacaacaaatacaactgaacaagtgattttgagtaagattgaagaaagtttgaaaaatgcccaaaaaccagtagtgattgcaggacacgaagtaattagttttggtttagaaaaaacggtaactcagtttgtttcagaaacaaaactaccgattacgacactaaattttggtaaaagtgctgttgatgaatctttgccctcatttttaggaatatataacgggaaactttcagaaatcagtcttaaaaattttgtggagtccgcagactttatcctaatgcttggagtgaagcttacggactcctcaacaggtgcattcacacatcatttagatgaaaataaaatgatttcactaaacatagatgaaggaataattttcaataaagtggtagaagattttgattttagagcagtggtttcttctttatcagaattaaaaggaatagaatatgaaggacaatatattgataagcaatatgaagaatttattccatcaagtgctcccttatcacaagaccgtctatggcaggcagttgaaagtttgactcaaagcaatgaaacaatcgttgctgaacaaggaacctcattttttggagcttcaacaattttcttaaaatcaaatagtcgttttattggacaacctttatggggttctattggatatacttttccagcggctttaggaagccaaattgcggataaagagagcagacaccttttatttattggtgatggttcacttcaacttaccgtacaagaattaggactatcaatcagagaaaaactcaatccaatttgttttatcataaataatgatggttatacagttgaaagagaaatccacggacctactcaaagttataacgacattccaatgtggaattactcgaaattaccagaaacatttggagcaacagaagatcgtgtagtatcaaaaattgttagaacagagaatgaatttgtgtctgtcatgaaagaagcccaagcagatgtcaatagaatgtattggatagaactagttttggaaaaagaagatgcgccaaaattactgaaaaaaatgggtaaattatttgctgagcaaaataaatag

SEQ ID NO: 115 - protein, 547 aa, Lactococcus lactis
MYTVGDYLLDRLHELGIEEIFGVPGDYNLQFLDQIISREDMKWIGNANELNASYMADGYARTKKAAAFLTTFGVGELSAINGLAGSYAENLPVVEIVGSPTSKVQNDGKFVHHTLADGDFKHFMKMHEPVTAARTLLTAENATYEIDRVLSQLLKERKPVYINLPVDVAAAKAEKPALSLEKESSTTNTTEQVILSKIEESLKNAQKPVVIAGHEVISFGLEKTVTQFVSETKLPITTLNFGKSAVDESLPSFLGIYNGKLSEISLKNFVESADFILMLGVKLTDSSTGAFTHHLDENKMISLNIDEGIIFNKVVEDFDFRAVVSSLSELKGIEYEGQYIDKQYEEFIPSSAPLSQDRLWQAVESLTQSNETIVAEQGTSFFGASTIFLKSNSRFIGQPLWGSIGYTFPAALGSQIADKESRHLLFIGDGSLQLTVQELGLSIREKLNPICFIINNDGYTVEREIHGPTQSYNDIPMWNYSKLPETFGATEDRVVSKIVRTENEFVSVMKEAQADVNRMYWIELVLEKEDAPKLLKKMGKLFAEQNK

SEQ ID NO: 116 - DNA, 1644 bp, Artificial
  Lactococcus lactis branched chain alpha- ketoacid decarboxylase KdcA codon optimised gene
atgtatactgttggtgattatctgctggaccgtctgcatgaactgggcattgaagaaatcttcggtgtcccaggcgactacaacctgcagttcctggaccagatcatctcccgcgaagatatgaaatggatcggtaacgcaaacgagctgaacgcgtcttatatggctgatggttatgctcgcaccaaaaaggctgcggcctttctgaccacctttggtgtgggcgagctgagcgcgatcaacggcctggcaggttcctacgctgagaacctgccggtagtagaaatcgttggttccccgacctctaaggttcagaacgacggcaaattcgtacatcacaccctggcggacggcgattttaagcactttatgaaaatgcacgaaccggtcaccgccgctcgcactctgctgaccgcggaaaacgcaacgtacgagatcgatcgtgtactgtcccagctgctgaaagaacgtaaaccggtgtatatcaatctgccggttgatgtcgctgcggccaaagcagagaaaccggcactgtccctggagaaggagagctccactactaacaccaccgaacaggttatcctgtccaaaattgaagaatctctgaaaaacgcacagaaaccggtggttatcgcaggtcacgaggttatctccttcggcctggagaaaactgttactcaattcgtctctgaaacgaaactgccgatcacgaccctgaactttggcaagtccgcagttgacgaatctctgccttctttcctgggcatttacaacggcaaactgtccgagatctccctgaagaacttcgtagaatccgctgactttatcctgatgctgggtgtgaaactgaccgactcctctaccggtgcgttcacgcaccatctggatgaaaacaaaatgatcagcctgaacatcgacgagggtatcatcttcaacaaggtagttgaagatttcgacttccgtgctgttgtcagcagcctgtccgagctgaaaggcattgagtacgagggtcaatacatcgataaacagtacgaagagtttattccgtcttctgcaccgctgagccaggaccgcctgtggcaggcagttgagtccctgacgcagtccaacgaaactatcgtagcggaacaaggtacctctttcttcggtgcttctaccatctttctgaagtccaactctcgctttatcggtcagccgctgtggggttctatcggttacacgttcccggctgcgctgggtagccagatcgctgataaagagtctcgtcatctgctgttcatcggtgatggttccctgcagctgactgtacaggaactgggtctgtctatccgtgaaaaactgaacccgatttgttttatcatcaataacgatggctacactgttgagcgtgaaattcatggtccgactcagtcttacaacgatattccgatgtggaactactctaaactgccggaaaccttcggtgcaactgaggatcgcgtcgtgagcaagattgtgcgtactgagaacgagttcgtatctgttatgaaagaggcgcaggcagatgtgaaccgcatgtactggatcgaactggttctggaaaaagaggatgcaccgaaactgctgaagaaaatgggtaaactgtttgcggagcagaacaagtaa

SEQ ID NO: 117 - DNA, 1647 bp, Lactococcus lactis
  CDS (1)..(1647)
atgtatacagtaggagattacctattagaccgattacacgagttaggaattgaagaaatttttggagtccctggagactataacttacaatttttagatcaaattatttcccacaaggatatgaaatgggtcggaaatgctaatgaattaaatgcttcatatatggctgatggctatgctcgtactaaaaaagctgccgcatttcttacaacctttggagtaggtgaattgagtgcagttaatggattagcaggaagttacgccgaaaatttaccagtagtagaaatagtgggatcacctacatcaaaagttcaaaatgaaggaaaatttgttcatcatacgctggctgacggtgattttaaacactttatgaaaatgcacgaacctgttacagcagctcgaactttactgacagcagaaaatgcaaccgttgaaattgaccgagtactttctgcactattaaaagaaagaaaacctgtctatatcaacttaccagttgatgttgctgctgcaaaagcagagaaaccctcactccctttgaaaaaggaaaactcaacttcaaatacaagtgaccaagaaattttgaacaaaattcaagaaagcttgaaaaatgccaaaaaaccaatcgtgattacaggacatgaaataattagttttggcttagaaaaaacagtcactcaatttatttcaaagacaaaactacctattacgacattaaactttggtaaaagttcagttgatgaagccctcccttcatttttaggaatctataatggtacactctcagagcctaatcttaaagaattcgtggaatcagccgacttcatcttgatgcttggagttaaactcacagactcttcaacaggagccttcactcatcatttaaatgaaaataaaatgatttcactgaatatagatgaaggaaaaatatttaacgaaagaatccaaaattttgattttgaatccctcatctcctctctcttagacctaagcgaaatagaatacaaaggaaaatatatcgataaaaagcaagaagactttgttccatcaaatgcgcttttatcacaagaccgcctatggcaagcagttgaaaacctaactcaaagcaatgaaacaatcgttgctgaacaagggacatcattctttggcgcttcatcaattttcttaaaatcaaagagtcattttattggtcaacccttatggggatcaattggatatacattcccagcagcattaggaagccaaattgcagataaagaaagcagacaccttttatttattggtgatggttcacttcaacttacagtgcaagaattaggattagcaatcagagaaaaaattaatccaatttgctttattatcaataatgatggttatacagtcgaaagagaaattcatggaccaaatcaaagctacaatgatattccaatgtggaattactcaaaattaccagaatcgtttggagcaacagaagatcgagtagtctcaaaaatcgttagaactgaaaatgaatttgtgtctgtcatgaaagaagctcaagcagatccaaatagaatgtactggattgagttaattttggcaaaagaaggtgcaccaaaagtactgaaaaaaatgggcaaactatttgctgaacaaaataaatcataa

SEQ ID NO: 118 - protein, 548 aa, Lactococcus lactis
MYTVGDYLLDRLHELGIEEIFGVPGDYNLQFLDQIISHKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENSTSNTSDQEILNKIQESLKNAKKPIVITGHEIISFGLEKTVTQFISKTKLPITTLNFGKSSVDEALPSFLGIYNGTLSEPNLKEFVESADFILMLGVKLTDSSTGAFTHHLNENKMISLNIDEGKIFNERIQNFDFESLISSLLDLSEIEYKGKYIDKKQEDFVPSNALLSQDRLWQAVENLTQSNETIVAEQGTSFFGASSIFLKSKSHFIGQPLWGSIGYTFPAALGSQIADKESRHLLFIGDGSLQLTVQELGLAIREKINPICFIINNDGYTVEREIHGPNQSYNDIPMWNYSKLPESFGATEDRVVSKIVRTENEFVSVMKEAQADPNRMYWIELILAKEGAPKVLKKMGKLFAEQNKS

SEQ ID NO: 119 - DNA, 1647 bp, Artificial
  Lactococcus lactis  -ketoisovalerate decarboxylase KivD codon optimised gene
atgtatactgttggtgattacctgctggatcgtctgcatgaactgggcatcgaggaaattttcggcgtacctggtgactataacctgcagttcctggatcagatcatttcccacaaagatatgaaatgggttggtaacgcgaacgagctgaatgcaagctacatggctgacggttatgcacgcaccaagaaagctgcggcgttcctgactacttttggcgtcggcgagctgtctgcggtaaacggtctggccggctcctacgcggaaaacctgccggtagtagaaatcgtcggttccccgacctctaaagttcagaacgagggtaaattcgtgcaccatactctggccgatggtgacttcaaacacttcatgaagatgcacgaaccggtcactgctgctcgtacgctgctgaccgcggaaaatgcgactgtcgagattgatcgtgtactgagcgcactgctgaaagaacgcaagcctgtatacatcaacctgccggttgatgtcgcggccgccaaagcggaaaaaccatctctgccgctgaaaaaggagaacagcacctctaacaccagcgaccaggaaatcctgaacaagatccaggagtctctgaagaacgctaaaaagccgatcgtaatcaccggccatgagattatctctttcggtctggagaaaactgtcacccagttcatcagcaaaaccaaactgccgatcaccaccctgaacttcggtaaatcctccgttgacgaagcgctgccgtcctttctgggtatttacaacggcactctgtctgagccgaacctgaaagagttcgtggagtctgcggattttatcctgatgctgggcgtgaaactgacggattcctccaccggtgcattcacccaccacctgaatgagaataaaatgatctctctgaacattgatgagggcaaaatcttcaacgagcgtattcagaacttcgatttcgaatccctgatctcctccctgctggatctgtccgagattgaatataaaggcaaatacattgataagaagcaagaggacttcgtaccgtctaacgcgctgctgagccaggaccgtctgtggcaagctgtggaaaacctgacccagtccaacgaaaccatcgtggcggaacagggtacctccttcttcggtgctagctctatcttcctgaaatctaaaagccacttcatcggtcagccactgtggggctctattggctacaccttcccggcagcgctgggttcccaaatcgcagacaaagaatcccgccacctgctgttcattggtgacggctctctgcaactgaccgtacaggagctgggtctggcgattcgtgagaaaatcaacccgatttgtttcatcatcaacaacgatggctacactgttgagcgtgagatccacggcccgaaccagtcctacaacgacattccgatgtggaactactctaaactgccggaatccttcggtgcgactgaagaccgtgtcgtaagcaagatcgtccgtaccgaaaacgaattcgtgtctgtcatgaaagaagcacaggcggacccgaaccgcatgtactggatcgagctgattctggctaaagagggcgcgccaaaagtactgaaaaagatgggtaaactgttcgcagaacagaacaaatcctaa

SEQ ID NO: 120 - DNA, 3696 bp, Mycobacterium tuberculosis
  CDS (1)..(3696)
gtggccaacataagttcaccattcgggcaaaacgaatggctggtcgaagagatgtaccgcaagttccgcgacgacccctcctcggtcgatcccagctggcacgagttcctggttgactacagccccgaacccacctcccaaccagctgccgaaccaacccgggttacctcgccactcgttgccgagcgggccgctgcggccgccccgcaggcaccccccaagccggccgacaccgcggccgcgggcaacggcgtggtcgccgcactggccgccaaaactgccgttcccccgccagccgaaggtgacgaggtagcggtgctgcgcggcgccgccgcggccgtcgtcaagaacatgtccgcgtcgttggaggtgccgacggcgaccagcgtccgggcggtcccggccaagctactgatcgacaaccggatcgtcatcaacaaccagttgaagcggacccgcggcggcaagatctcgttcacgcatttgctgggctacgccctggtgcaggcggtgaagaaattcccgaacatgaaccggcactacaccgaagtcgacggcaagcccaccgcggtcacgccggcgcacaccaatctcggcctggcgatcgacctgcaaggcaaggacgggaagcgttccctggtggtggccggcatcaagcggtgcgagaccatgcgattcgcgcagttcgtcacggcctacgaagacatcgtacgccgggcccgcgacggcaagctgaccactgaagactttgccggcgtgacgatttcgctgaccaatcccggaaccatcggcaccgtgcattcggtgccgcggctgatgcccggccagggcgccatcatcggcgtgggcgccatggaataccccgccgagtttcaaggcgccagcgaggaacgcatcgccgagctgggcatcggcaaattgatcactttgacctccacctacgaccaccgcatcatccagggcgcggaatcgggcgacttcctgcgcaccatccacgagttgctgctctcggatggcttctgggacgaggtcttccgcgaactgagcatcccatatctgccggtgcgctggagcaccgacaaccccgactcgatcgtcgacaagaacgctcgcgtcatgaacttgatcgcggcctaccgcaaccgcggccatctgatggccgataccgacccgctgcggttggacaaagctcggttccgcagtcaccccgacctcgaagtgctgacccacggcctgacgctgtgggatctcgatcgggtgttcaaggtcgacggctttgccggtgcgcagtacaagaaactgcgcgacgtgctgggcttgctgcgcgatgcctactgccgccacatcggcgtggagtacgcccatatcctcgaccccgaacaaaaggagtggctcgaacaacgggtcgagaccaagcacgtcaaacccactgtggcccaacagaaatacatcctcagcaagctcaacgccgccgaggcctttgaaacgttcctacagaccaagtacgtcggccagaagcggttctcgctggaaggcgccgaaagcgtgatcccgatgatggacgcggcgatcgaccagtgcgctgagcacggcctcgacgaggtggtcatcgggatgccgcaccggggccggctcaacgtgctggccaacatcgtcggcaagccgtactcgcagatcttcaccgagttcgagggcaacctgaatccgtcgcaggcgcacggctccggtgacgtcaagtaccacctgggcgccaccgggctgtacctgcagatgttcggcgacaacgacattcaggtgtcgctgaccgccaacccgtcgcatctggaggccgtcgacccggtgctggagggattggtgcgggccaagcaggatctgctcgaccacggaagcatcgacagcgacggccaacgggcgttctcggtggtgccgctgatgttgcatggcgatgccgcgttcgccggtcagggtgtggtcgccgagacgctgaacctggcgaatctgccgggctaccgcgtcggcggcaccatccacatcatcgtcaacaaccagatcggcttcaccaccgcgcccgagtattccaggtccagcgagtactgcaccgacgtcgcaaagatgatcggggcaccgatctttcacgtcaacggcgacgacccggaggcgtgtgtctgggtggcgcggttggcggtggacttccgacaacggttcaagaaggacgtcgtcatcgacatgctgtgctaccgccgccgcgggcacaacgagggtgacgacccgtcgatgaccaacccctacgtgtacgacgtcgtcgacaccaagcgcggggcccgcaaaagctacaccgaagccctgatcggacgtggcgacatctcgatgaaggaggccgaggacgcgctgcgcgactaccagggccagctggaacgggtgttcaacgaagtgcgcgagctggagaagcacggtgtgcagccgagcgagtcggtcgagtccgaccagatgattcccgcggggctggccactgcggtggacaagtcgctgctggcccggatcggcgatgcgttcctcgccttgccgaacggcttcaccgcgcacccgcgagtccaaccggtgctggagaagcgccgggagatggcctatgaaggcaagatcgactgggcctttggcgagctgctggcgctgggctcgctggtggccgaaggcaagctggtgcgcttgtcggggcaggacagccgccgcggcaccttctcccagcggcattcggttctcatcgaccgccacactggcgaggagttcacaccactgcagctgctggcgaccaactccgacggcagcccgaccggcggaaagttcctggtctacgactcgccactgtcggagtacgccgccgtcggcttcgagtacggctacactgtgggcaatccggacgccgtggtgctctgggaggcgcagttcggcgacttcgtcaacggcgcgcagtcgatcatcgacgagttcatcagctccggtgaggccaagtggggccaattgtccaacgtcgtgctgctgttaccgcacgggcacgaggggcagggacccgaccacacttctgcccggatcgaacgcttcttgcagttgtgggcggaaggttcgatgaccatcgcgatgccgtcgactccgtcgaactacttccacctgctacgccggcatgccctggacggcatccaacgcccgctgatcgtgttcacgcccaagtcgatgttgcgtcacaaggccgccgtcagcgaaatcaaggacttcaccgagatcaagttccgctcagtgctggaggaacccacctatgaggacggcatcggagaccgcaacaaggtcagccggatcctgctgaccagtggcaagctgtattacgagctggccgcccgcaaggccaaggacaaccgcaatgacctcgcgatcgtgcggcttgaacagctcgccccgctgcccaggcgtcgactgcgtgaaacgctggaccgctacgagaacgtcaaggagttcttctgggtccaagaggaaccggccaaccagggtgcgtggccgcgattcgggctcgaactacccgagctgctgcctgacaagttggccgggatcaagcgaatctcgcgccgggcgatgtcagccccgtcgtcaggctcgtcgaaggtgcacgccgtcgaacagcaggagatcctcgacgaggcgttcggctga

SEQ ID NO: 121 - protein, 1231 aa, Mycobacterium tuberculosis
VANISSPFGQNEWLVEEMYRKFRDDPSSVDPSWHEFLVDYSPEPTSQPAAEPTRVTSPLVAERAAAAAPQAPPKPADTAAAGNGVVAALAAKTAVPPPAEGDEVAVLRGAAAAVVKNMSASLEVPTATSVRAVPAKLLIDNRIVINNQLKRTRGGKISFTHLLGYALVQAVKKFPNMNRHYTEVDGKPTAVTPAHTNLGLAIDLQGKDGKRSLVVAGIKRCETMRFAQFVTAYEDIVRRARDGKLTTEDFAGVTISLTNPGTIGTVHSVPRLMPGQGAIIGVGAMEYPAEFQGASEERIAELGIGKLITLTSTYDHRIIQGAESGDFLRTIHELLLSDGFWDEVFRELSIPYLPVRWSTDNPDSIVDKNARVMNLIAAYRNRGHLMADTDPLRLDKARFRSHPDLEVLTHGLTLWDLDRVFKVDGFAGAQYKKLRDVLGLLRDAYCRHIGVEYAHILDPEQKEWLEQRVETKHVKPTVAQQKYILSKLNAAEAFETFLQTKYVGQKRFSLEGAESVIPMMDAAIDQCAEHGLDEVVIGMPHRGRLNVLANIVGKPYSQIFTEFEGNLNPSQAHGSGDVKYHLGATGLYLQMFGDNDIQVSLTANPSHLEAVDPVLEGLVRAKQDLLDHGSIDSDGQRAFSVVPLMLHGDAAFAGQGVVAETLNLANLPGYRVGGTIHIIVNNQIGFTTAPEYSRSSEYCTDVAKMIGAPIFHVNGDDPEACVWVARLAVDFRQRFKKDVVIDMLCYRRRGHNEGDDPSMTNPYVYDVVDTKRGARKSYTEALIGRGDISMKEAEDALRDYQGQLERVFNEVRELEKHGVQPSESVESDQMIPAGLATAVDKSLLARIGDAFLALPNGFTAHPRVQPVLEKRREMAYEGKIDWAFGELLALGSLVAEGKLVRLSGQDSRRGTFSQRHSVLIDRHTGEEFTPLQLLATNSDGSPTGGKFLVYDSPLSEYAAVGFEYGYTVGNPDAVVLWEAQFGDFVNGAQSIIDEFISSGEAKWGQLSNVVLLLPHGHEGQGPDHTSARIERFLQLWAEGSMTIAMPSTPSNYFHLLRRHALDGIQRPLIVFTPKSMLRHKAAVSEIKDFTEIKFRSVLEEPTYEDGIGDRNKVSRILLTSGKLYYELAARKAKDNRNDLAIVRLEQLAPLPRRRLRETLDRYENVKEFFWVQEEPANQGAWPRFGLELPELLPDKLAGIKRISRRAMSAPSSGSSKVHAVEQQEILDEAFG

SEQ ID NO: 122 - DNA, 3696 bp, Artificial
  Mycobacterium tuberculosis  -ketoglutarate decarboxylase Kgd codon optimised gene
atggctaatatctcctctccgtttggtcagaatgaatggctggtagaagaaatgtaccgtaaattccgcgatgacccgtcctctgtggacccgtcctggcatgaattcctggtagactacagcccggagccgaccagccaaccggcagcggaaccaacccgcgttacttctccgctggtagcggaacgtgcagctgctgccgcgcctcaggcgccgcctaaaccggcggatactgccgcagccggtaacggtgtggtggccgcactggctgctaagactgcggttccgccgccagcagaaggcgatgaagttgcagtcctgcgcggtgcggcggctgcagtggtgaaaaacatgagcgcgtccctggaggtaccgaccgccacgagcgtgcgcgcggtccctgctaaactgctgattgataaccgtattgtgatcaacaaccagctgaaacgtacccgtggtggcaagatctccttcactcatctgctgggttatgcactggtacaagcggttaagaaattccctaacatgaaccgtcattacactgaggtcgacggtaaaccgacggctgttactccggcacacacgaacctgggcctggcgatcgacctgcaaggtaaagatggtaagcgctccctggtagttgcgggtattaaacgttgcgaaaccatgcgtttcgcacaattcgtaaccgcctacgaggacattgtccgccgtgctcgtgatggcaaactgaccaccgaagattttgcgggcgttactattagcctgaccaacccaggcaccatcggcaccgtgcacagcgtacctcgtctgatgccgggccaaggtgcgattatcggtgtgggtgccatggagtacccggcagaatttcagggtgcttctgaagagcgcatcgccgagctgggtattggtaaactgatcaccctgacttctacctatgaccaccgcatcattcagggcgcagaatccggtgacttcctgcgcactattcacgaactgctgctgtccgacggtttctgggatgaagtttttcgtgaactgagcatcccatatctgccagttcgctggtccaccgacaatccggactctatcgttgacaaaaacgctcgcgtaatgaacctgatcgctgcttatcgtaatcgtggtcacctgatggctgatacggatccgctgcgcctggataaagctcgtttccgttcccacccggacctggaagtgctgacccatggtctgactctgtgggatctggaccgcgtgttcaaagtagatggtttcgcgggtgctcagtacaagaagctgcgtgacgtgctgggtctgctgcgtgatgcgtactgtcgtcacattggtgtggagtacgcccacattctggatccggaacagaaagaatggctggagcagcgtgtcgagaccaaacacgtaaaaccgaccgtagcgcagcagaaatatatcctgtccaaactgaacgccgccgaggctttcgaaactttcctgcagaccaagtacgtgggccagaaacgcttcagcctggagggtgcggaaagcgttattccgatgatggatgcagctatcgatcagtgcgcggaacatggtctggatgaagtcgttatcggtatgccgcaccgtggtcgcctgaacgtactggcaaacatcgtcggtaaaccatattctcagatcttcacggaattcgagggcaacctgaacccgtcccaagcccacggctccggcgacgtaaaatatcatctgggtgctaccggcctgtatctgcagatgttcggtgataacgacatccaggtatctctgactgctaacccgagccacctggaggcggttgatcctgttctggaaggtctggttcgcgccaaacaggatctgctggaccacggctctatcgacagcgatggccagcgtgcattcagcgttgtaccgctgatgctgcatggcgacgcggcgttcgccggtcagggtgtcgtagcagaaactctgaacctggcgaacctgcctggctatcgcgtgggtggcaccattcacatcatcgttaacaaccaaatcggtttcaccacggcaccggagtatagccgttctagcgaatattgcaccgacgtagccaaaatgatcggtgcgccgatcttccatgtaaacggtgacgatccagaggcctgcgtgtgggtggctcgtctggccgtagacttccgccagcgttttaagaaagatgtggttatcgacatgctgtgctaccgccgtcgtggtcacaacgaaggtgatgatccgtctatgactaacccgtatgtctatgacgtggtggacaccaagcgtggtgcacgcaaatcttacacggaggccctgatcggtcgtggcgacatctctatgaaagaagcggaagacgctctgcgtgattaccagggtcagctggaacgtgtgttcaatgaggtgcgtgagctggaaaagcacggcgtacaaccgtccgaatccgtagagtccgatcagatgatccctgctggtctggcaactgctgttgataaaagcctgctggcgcgtatcggcgacgcattcctggcgctgccgaatggctttaccgcgcacccgcgcgtacagccggtactggaaaaacgtcgtgaaatggcctacgaaggtaaaatcgattgggccttcggtgagctgctggccctgggctctctggtggctgagggcaagctggtacgcctgagcggccaggactcccgtcgcggcactttttctcagcgtcacagcgtcctgatcgatcgtcacaccggcgaagaattcacgccgctgcaactgctggctactaactccgatggtagcccgaccggtggtaagttcctggtgtacgattccccgctgtccgaatatgctgcagttggtttcgagtatggttacaccgttggcaacccggacgcagtggttctgtgggaagcgcagttcggcgatttcgttaacggtgcccagtccattatcgatgagtttattagcagcggcgaggccaaatggggccagctgtctaacgttgtgctgctgctgcctcacggccacgagggtcaaggcccggaccacacctccgcccgtatcgaacgcttcctgcagctgtgggctgaaggctctatgaccatcgcgatgccgtctaccccaagcaactacttccacctgctgcgtcgccacgcactggacggcattcagcgcccgctgatcgttttcaccccaaaatccatgctgcgccacaaagcagctgtttctgaaatcaaagattttacggaaattaaattccgttctgtgctggaagaaccaacctacgaagacggtattggcgaccgcaacaaggtaagccgtatcctgctgacctccggcaaactgtactacgagctggcagcacgtaaggcaaaagataaccgcaacgacctggccatcgtccgcctggaacagctggcgccactgccacgccgtcgcctgcgtgaaaccctggatcgctacgaaaacgtaaaagaattcttctgggtgcaggaagaaccggcaaaccagggtgcgtggccgcgctttggtctggaactgccggaactgctgccggataaactggcaggtatcaagcgcatcagccgtcgcgctatgagcgccccgtcttctggtagctctaaagtacacgctgtagaacagcaagagatcctggatgaggccttcggctaa

SEQ ID NO: 123 - DNA, 74 bp, Artificial
  Forward primer for amplification of Bacillus subtilis aminotransferase x
ggggacaagtttgtacaaaaaagcaggctaggaggaattaaccatgaaggttttagtcaatggccggctgattg

SEQ ID NO: 124 - DNA, 62 bp, Artificial
  Reverse primer for amplification of Bacillus subtilis aminotransferase x
ggggaccactttgtacaagaaagctgggtttatgaaatgctagcagcctgttgaatgctttc

SEQ ID NO: 125 - DNA, 82 bp, Artificial
  Forward primer for amplification of Bacillus subtilis aminotransferase y
ggggacaagtttgtacaaaaaagcaggctaggaggaattaaccatgactcatgatttgatagaaaaaagtaaaaagcacctc

SEQ ID NO: 126 - DNA, 57 bp, Artificial
  Reverse primer for amplification of Bacillus subtilis aminotransferase y
ggggaccactttgtacaagaaagctgggttcaatcttcaaggctcgtaacctcgtgg

SEQ ID NO: 127 - DNA, 64 bp, Artificial
  Forward primer for amplification of Rhodobacter sphaeroides aminotransferase
ggggacaagtttgtacaaaaaagcaggctaggaggaattaaccatgcccggttgcgggggcttg

SEQ ID NO: 128 - DNA, 51 bp, Artificial
  Reverse primer for amplification of Rhodobacter sphaeroides aminotransferase
ggggaccactttgtacaagaaagctgggttcagacggcggccggttctttc

SEQ ID NO: 129 - DNA, 78 bp, Artificial
  Forward primer for amplification of Legionella pneumophila aminotransferase
ggggacaagtttgtacaaaaaagcaggctaggaggaattaaccatgagtatcgcatttgttaacggcaagtattgttg

SEQ ID NO: 130 - DNA, 67 bp, Artificial
  Reverse primer for amplification of Legionella pneumophila aminotransferase
ggggaccactttgtacaagaaagctgggtttagtttactagttgttggtaggaatcattaattatcc

SEQ ID NO: 131 - DNA, 76 bp, Artificial
  Forward primer for amplification of Nitrosomonas europaea aminotransferase
ggggacaagtttgtacaaaaaagcaggctaggaggaattaaccatgatttacctcaatggcaaatttctgccgatg

SEQ ID NO: 132 - DNA, 50 bp, Artificial
  Reverse primer for amplification of Nitrosomonas europaea aminotransferase
ggggaccactttgtacaagaaagctgggtttactggcgtggagcatgccc

SEQ ID NO: 133 - DNA, 79 bp, Artificial
  Forward primer for amplification of Neisseria gonorrhoeae aminotransferase
ggggacaagtttgtacaaaaaagcaggctaggaggaattaaccatgaggataaatatgaaccgtaacgaaattttattc

SEQ ID NO: 134 - DNA, 56 bp, Artificial
  Reverse primer for amplification of Neisseria gonorrhoeae aminotransferase
ggggaccactttgtacaagaaagctgggttcatgcagccatcgccttgaacacttc

SEQ ID NO: 135 - DNA, 66 bp, Artificial
  Forward primer for amplification of Pseudomonas aeruginosa aminotransferase
ggggacaagtttgtacaaaaaagcaggctaggaggaattaaccatgtcgatggccgatcgtgatgg

SEQ ID NO: 136 - DNA, 53 bp, Artificial
  Reverse primer for amplification of Pseudomonas aeruginosa aminotransferase
ggggaccactttgtacaagaaagctgggtttacttgaccagggtacgccactc

SEQ ID NO: 137 - DNA, 67 bp, Artificial
  Forward primer for amplification of Rhodopseudomonas palustris aminotransferase
ggggacaagtttgtacaaaaaagcaggctaggaggaattaaccatgaagctgataccgtgccgcgcc

SEQ ID NO: 138 - DNA, 51 bp, Artificial
  Reverse primer for amplification of Rhodopseudomonas palustris aminotransferase
ggggaccactttgtacaagaaagctgggttcaggcgaccgcgcggatcacc

SEQ ID NO: 139 - DNA, 71 bp, Artificial
  Forward primer for amplification of Bacillus subtilis aminotransferase (gi16077991)
ggggacaagtttgtacaaaaaagcaggctaggaggaattaaccatggagatgatggggatggaaaacattc

SEQ ID NO: 140 - DNA, 65 bp, Artificial
  Reverse primer for amplification of Bacillus subtilis aminotransferase (gi16077991)
ggggaccactttgtacaagaaagctgggtttatatcgtttgaaagctttctttcaccgttttcac

SEQ ID NO: 141 - DNA, 66 bp, Artificial
  Forward primer for amplification of Pseudomonas aeruginosa aminotransferase (gi9951072)
ggggacaagtttgtacaaaaaagcaggctaggaggaattaaccatgaacgcaagactgcacgccac

SEQ ID NO: 142 - DNA, 48 bp, Artificial
  Reverse primer for amplification of Pseudomonas aeruginosa aminotransferase (gi9951072)
ggggaccactttgtacaagaaagctgggtttaccggtgaccggcgcgg

SEQ ID NO: 143 - DNA, 69 bp, Artificial
  Forward primer for amplification of Pseudomonas aeruginosa aminotransferase (gi9951630)
ggggacaagtttgtacaaaaaagcaggctaggaggaattaaccatgacaatgaatgacgagccgcagtc

SEQ ID NO: 144 - DNA, 49 bp, Artificial
  Reverse primer for amplification of Pseudomonas aeruginosa aminotransferase (gi9951630)
ggggaccactttgtacaagaaagctgggttcagacgctggcgcggatgg

SEQ ID NO: 145 - DNA, 1221 bp, Methanococcus jannaschii
atgacaaaagtgctggtgatgtttatggatttcttatttgagaacagctggaaagcagtttgtccctacaatccaaagttggatttaaaggacatttatatttatgacacaaccctaagagatggagagcaaaccccaggagtttgctttaccaaagaacaaaaattggagattgcaaggaagttggatgaacttggattaaagcagattgaagctggcttcccaatagtatctgaaagagaagcagatatagttaaaacaattgctaatgaagggctaaatgctgatatcttagctttatgcagggctttaaagaaagatatagataaagcaatagagtgcgatgtagatgggattattaccttcatagcaacatctcctctccacttaaaatataaattcaacaacaaaagcttagatgaaatattagagatgggagttgaggcagttgagtatgcaaaggaacatggcttatttgttgctttctctgcagaggatgcgacaagaacaccaatagaggacttgattaaagtgcataaagccgctgaagaggctggagcagatagggttcatatagcagacacaactggctgtgctaccccccaaagtatggagtttatatgtaaaacattgaaggagaacttaaaaaaggcacatattggagtgcattgtcacaacgactttggatttgcagttataaattcaatatatggtttaattggaggagctaaggcagtttcaacaacagttaatggtattggagagagggcagggaatgcagctttagaagagctaattatggctttaactgtcttgtatgatgttgatttgggattaaacttggaggttcttccagagttatgcagaatggttgaggaatactctggaataaagatgccaaagaacaaaccaatagttggagagcttgtatttgctcatgaaagtggaattcacgttgatgctgtcatagagaatccattaacctatgaacccttccttccagagaaaatagggcttaagagaaatattttgttagggaagcattctggatgcagagccgttgcctataagctaaaacttatgggaattgattacgatagagagatgttgtgcgagattgttaaaaaggttaaagagattagagaggaaggtaaatttataactgatgaagtctttaaggagattgttgaagaagttttaaggaagagaaataaaaattaa

SEQ ID NO: 146 - DNA, 513 bp, Methanococcus jannaschii
atgattattaagggaagagctcacaaatttggggatgatgtagatacagacgcaataattccaggaccttacttaaggactacagacccttacgagttagcttcacactgcatggcagggatagatgaaaacttcccgaaaaaggttaaggagggggatgtgatagttgctggagagaattttggttgtggttcaagtagggagcaggctgtaatagcaataaaatactgtggtattaaggctgtgatagcaaaaagctttgcaagaatattctatagaaatgcaataaacgttggattaataccaataatagcaaatacagatgaaattaaagacggagacatagtagagattgatttagataaagaagagattgtaataaccaataaaaacaaaacaataaagtgtgaaacaccaaaaggtttagaaagagaaatattggctgctggtggcttagtcaattatttaaaaaagagaaaactaatacaatcaaaaaaaggtgtaaaaacatga

SEQ ID NO: 147 - DNA, 1263 bp, Methanococcus jannaschii
ttgacattggtagagaagatactatcaaaaaaagttggttatgaagtttgtgcaggagatagcatagaggttgaagttgatttggcaatgacacacgatggaacaacacctttagcatacaaagctttaaaggaaatgagtgatagtgtttggaatccagataaaatagtcgttgcctttgaccacaatgttccaccaaacacagttaaagctgctgaaatgcaaaaattagctttggagtttgttaaaagatttggcattaaaaatttccataaaggtggagaaggcatctgtcatcaaatcttagctgaaaattatgttttgccaaacatgtttgtagctggtggagacagccatacatgcacacatggagcttttggagcttttgctactggctttggagctactgatatggcttacatctatgcaacaggagaaacatggattaaagtgccaaaaacaattagggtagatatagttggaaaaaatgaaaatgtttctgccaaagatattgttttaagggtttgtaaggaaattgggagaagaggagcaacatacatggctattgagtatggtggagaggttgttaaaaacatggacatggatggaaggctaactttatgcaacatggcaatagagatgggaggaaaaacaggagtgatagaggctgatgaaattacttatgattatttaaagaaagagagaggactttctgatgaggatatagctaaattaaaaaaagagagaataacagtaaatagagatgaagcaaactactataaggagatagaaattgacataacagatatggaagaacaagttgctgttccacaccacccagataacgtaaagccaattagtgatgttgaagggactgagataaatcaagtttttattgggagttgcacaaatggaaggttgagtgatttaagagaagcagctaaatatttaaaaggtagggaggttcataaagatgttaagctaattgttatcccggcatcaaaaaaggtatttttgcaagcgttaaaagagggtattatagatatctttgttaaagctggggcgatgatttgcactccgggatgcggaccttgcttaggagctcatcaaggggttttggctgagggagaaatttgtttatcaacaacaaacagaaactttaaaggaaggatggggcatataaatagctatatttacttggcatctccaaagattgccgcaataagtgcagttaagggatatataaccaacaaattggattaa

SEQ ID NO: 148 - DNA, 1044 bp, Methanococcus jannaschii
atgatgaaggtgtgtgttatagaaggggatggaataggaaaagaagtgattccagaggccataaaaatattaaatgagttgggagagtttgaaataataaaaggagaggcaggattagaatgtttaaaaaaatatggtaatgcacttccagaggatacaatagaaaaagctaaagaggcagatattattttgtttggggctataacctcaccaaagccaggggaagttcaaaattataaaagccctataataacgttgaggaagatgtttcatttatatgcaaatgtaagaccaataaacaactttggaattggacaattaattgggaaaattgcagattatgaattcttaaatgctaagaatattgatatagttattataagagagaatacggaagatttatatgttggtagagagagattagaaaatgatacagcaatagctgagagggttataacaagaaagggtagcgagagaataataagatttgcatttgaatatgctataaaaaataataggaaaaaggtatcttgcatccataaagctaatgttttaagaataactgatggtttattcttagaggtttttaatgaaataaaaaaacattataatatagaggcagatgattatttagttgattcaacagctatgaacttaataaaacatcctgaaaaatttgatgttattgttacaacaaacatgtttggggatattttatcagatgaggcatctgcattaattggaggacttggtttagctccttcagcaaatataggagatgataaagcattatttgagccagttcatggttcagctccagatatagctgggaaaggtatagcaaatccaatggcatctatattaagtattgctatgctttttgattatattggagagaaagaaaagggagatttgattagagaggcagtgaaatactgcttaataaacaaaaaagttactcctgacttgggaggggatttaaagacaaaagatgttggagacgaaattctaaattacattagaaagaagttaaagggatattga

SEQ ID NO: 149 - DNA, 1155 bp, Azotobacter vinelandii
atggctagcgtgatcatcgacgacactaccctgcgtgacggtgaacagagtgccggggtcgccttcaatgccgacgagaagatcgctatcgcccgcgcgctcgccgaactgggcgtgccggagttggagatcggcattcccagcatgggcgaggaagagcgcgaggtgatgcacgccatcgccggtctcggcctgtcgtctcgcctgctggcctggtgccggctatgcgacgtcgatctcgcggcggcgcgctccaccggggtgaccatggtcgacctttcgctgccggtctccgacctgatgctgcaccacaagctcaatcgcgatcgcgactgggccttgcgcgaagtggccaggctggtcggcgaagcgcgcatggccgggctcgaggtgtgcctgggctgcgaggacgcctcgcgggcggatctggagttcgtcgtgcaggtgggcgaagtggcgcaggccgccggcgcccgtcggctgcgcttcgccgacaccgtcggggtcatggagcccttcggcatgctcgaccgcttccgtttcctcagccggcgcctggacatggagctggaagtgcacgcccacgatgatttcgggctggccacggccaacaccctggccgcggtgatgggcggggcgactcatatcaacaccacggtcaacgggctcggcgagcgtgccggcaacgccgcgctggaagagtgcgtgctggcgctcaagaacctccacggtatcgacaccggtatcgatacccgcggcatcccggccatctccgcgctggtcgagcgggcctcggggcgccaggtggcctggcagaagagcgtggtcggcgccggggtgttcactcacgaggccggtatccacgtcgacggactgctcaagcatcggcgcaactacgaggggctgaatcccgacgaactcggtcgcagccacagtctggtgctgggcaagcattccggggcgcacatggtgcgcaacacgtaccgcgatctgggtatcgagctggcggactggcagagccaagcgctgctcggccgcatccgtgccttctccaccaggaccaagcgcagcccgcagcctgccgagctgcaggatttctatcggcagttgtgcgagcaaggcaatcccgaactggccgcaggaggaatggcatga

SEQ ID NO: 150 - DNA, 30 bp, Artificial
  Avine-WT-R-BamHI
aaattggatcctcatgccattcctcctgcg

SEQ ID NO: 151 - DNA, 75 bp, Artificial
  Avine-WT-F-SacI
aaattgagctctttctccatacccgtttttttgggctaacaggaggaattaaccatggctagcgtgatcatcgac

SEQ ID NO: 152 - DNA, 31 bp, Artificial
  Avine-WT-R-HindIII
aaattaaagctttcatgccattcctcctgcg

SEQ ID NO: 153 - DNA, 76 bp, Artificial
  Avine-WT-F-HindIII
aaattaaagctttttctccatacccgtttttttgggctaacaggaggaattaaccatggctagcgtgatcatcgac

SEQ ID NO: 154 - DNA, 21 bp, Artificial
  AksA-Avine-F
atggctagcgtgatcatcgac

SEQ ID NO: 155 - DNA, 32 bp, Artificial
  AksA-Avine-R1
aaattggcgcgcctcatgccattcctcctgcg

SEQ ID NO: 156 - DNA, 32 bp, Artificial
  Pgal2-F2
aaattgttaactccagaaggcacatctattac

SEQ ID NO: 157 - DNA, 49 bp, Artificial
  Pgal2-R
cgtcgatgatcacgctagccattatgaaagcctccttttttttattatg

SEQ ID NO: 158 - DNA, 207 bp, Artificial
  mtSP
atggcctccactcgtgtcctcgcctctcgcctggcctcccagatggctgcttccgccaaggttgcccgccctgctgtccgcgttgctcaggtcagcaagcgcaccatccagactggctcccccctccagaccctcaagcgcacccagatgacctccatcgtcaacgccaccacccgccaggctttccagaagcgcgcctactcttcc

SEQ ID NO: 159 - DNA, 29 bp, Artificial
  pF113-F-NsiI
aaattatgcatacagcatggcctgcaacg

SEQ ID NO: 160 - DNA, 31 bp, Artificial
  pF113-R-AgeI
aaattaccggtcagggttattgtctcatgag

SEQ ID NO: 161 - DNA, 32 bp, Artificial
  AT-Vfl_for_Ec
aaatttggtaccgctaggaggaattaaccatg

SEQ ID NO: 162 - DNA, 33 bp, Artificial
  Kdc_for_Ec
aaatttactagtggctaggaggaattacatatg

SEQ ID NO: 163 - DNA, 35 bp, Artificial
  Kdc_rev_Ec
aaatttaagcttattacttgttctgctccgcaaac

SEQ ID NO: 164 - DNA, 57 bp, Artificial
  AT-Vfl-F
aaatttactagtaagaatttttgaggaggcaatataaatgaataaaccacagtcttg

SEQ ID NO: 165 - DNA, 32 bp, Artificial
  AT-Vfl-R
aaatttggatcctacaagaaagctgggtttac

SEQ ID NO: 166 - DNA, 33 bp, Artificial
  AT-Vfl_rev_Ec
aaatttactagtaagctgggtttacgcgacttc

SEQ ID NO: 167 - DNA, 1221 bp, Escherichia coli
atgaccaaagttctggtaatgttcatggacttcctgttcgaaaactcctggaaagcggtttgcccgtacaacccgaaactggatctgaaagacatctacatctacgacaccactctgcgtgacggtgaacagactccgggcgtttgcttcaccaaagagcagaagctggaaatcgctcgtaagctggacgaactgggtctgaagcagatcgaagctggcttcccgatcgtttctgaacgtgaagctgacatcgttaaaactatcgctaacgaaggtctgaacgctgacatcctggcactgtgccgtgcgctgaagaaagacatcgacaaagcaatcgaatgcgacgttgacggtatcatcactttcatcgcaacttctccgctgcacctgaaatacaaattcaacaacaaatctctggatgaaatcctggaaatgggcgttgaagcggtagaatacgctaaagagcacggtctgttcgttgcattctctgcagaagatgcaactcgtactccgatcgaagatctgatcaaagttcacaaagcagctgaagaagcgggtgctgaccgcgttcacatcgctgacaccactggctgcgcaactccgcagtctatggaattcatctgcaaaactctgaaagaaaacctgaagaaagcacacatcggcgtacactgccacaacgacttcggtttcgctgttatcaactccatctacggtctgatcggtggtgcgaaagcggtatctactaccgttaacggtatcggtgaacgtgctggtaacgctgcactggaagagctgatcatggcgctgaccgtactgtacgacgttgacctgggtctgaacctggaagttctgccggaactgtgccgtatggttgaagaatactccggtatcaagatgccgaaaaacaagccaatcgttggtgaactggtattcgctcacgaatccggtatccacgttgacgctgttatcgaaaacccgctgacttacgaaccgttcctgccggaaaaaatcggtctgaaacgtaacatcctgctgggtaagcactctggttgccgtgctgttgcttacaagctgaaactgatgggtatcgactacgaccgtgaaatgctgtgcgaaatcgttaagaaagttaaagaaatccgtgaagaaggtaaattcatcactgacgaagttttcaaagagatcgttgaagaagttctgcgtaagcgtaacaaaaactaa

SEQ ID NO: 168 - DNA, 1044 bp, Escherichia coli
atgatgaaagtttgcgttatcgaaggtgacggtatcggtaaagaagttatcccggaagctatcaagatcctgaacgaactgggtgaattcgaaatcatcaaaggtgaagcgggtctggaatgcctgaagaaatacggtaacgcactgccagaagataccatcgaaaaagcgaaagaagctgacatcatcctgttcggtgcaatcacttctccgaagccgggtgaagttcagaactacaaatctccgatcatcactctgcgtaagatgttccacctgtacgctaacgtacgtccgatcaacaacttcggtatcggtcagctgatcggtaagatcgctgactacgagttcctgaacgctaaaaacatcgacatcgttatcatccgtgaaaacactgaagatctgtacgttggtcgtgaacgtctggaaaacgacactgctatcgctgagcgcgttatcactcgtaaaggttctgaacgtatcatccgcttcgcattcgaatacgcaatcaaaaacaaccgtaagaaagtttcctgcatccacaaagctaacgtactgcgtatcactgacggtctgttcctggaagtattcaacgaaatcaagaaacactacaacatcgaagctgacgactacctggttgactccactgcaatgaacctgatcaagcacccggaaaaattcgacgttatcgttaccactaacatgttcggtgacatcctgtctgacgaagcgtctgcactgatcggtggtctgggtctggcaccgtctgctaacatcggtgacgacaaagcgctgttcgaaccggttcacggttctgcaccggatatcgctggtaaaggtatcgctaacccgatggcttctatcctgtctatcgcgatgctgttcgactacatcggtgaaaaagagaaaggcgacctgatccgtgaagcggtaaaatactgcctgatcaacaagaaagttactccggatctgggtggtgacctgaaaaccaaagacgttggtgacgaaatcctgaactacatccgtaagaaactgaaaggttactaa

SEQ ID NO: 169 - DNA, 1263 bp, Escherichia coli
atgactctggttgagaagatcctctccaagaaagttggttacgaagtttgcgcaggcgactccatcgaagttgaagttgacctggcgatgactcacgacggtactactccgctggcttacaaagcgctgaaagagatgtctgactccgtatggaacccggacaagatcgttgttgcattcgaccacaacgtaccgccgaacaccgttaaagcagctgaaatgcagaagctggcgctggaattcgttaagcgcttcggtatcaaaaacttccacaaaggtggtgaaggtatctgccaccagatcctggctgaaaactacgttctgccgaacatgttcgttgctggcggcgactctcacacctgtactcacggtgcattcggtgcattcgcaactggcttcggtgcaactgacatggcttacatctacgcaactggcgaaacctggatcaaagttccgaaaactatccgcgttgatatcgttggtaaaaacgaaaacgtatctgcgaaagacatcgttctgcgcgtttgcaaagaaatcggtcgtcgcggtgcaacttacatggctatcgaatacggtggtgaagttgttaaaaacatggacatggacggtcgtctgactctgtgcaacatggctatcgaaatgggtggtaaaactggcgttatcgaagctgacgaaatcacttacgactacctgaagaaagagcgtggtctgtctgacgaagatatcgctaaactgaagaaagagcgtatcaccgttaaccgtgacgaagctaactactacaaagaaatcgaaatcgacatcactgacatggaagaacaggttgctgtaccgcaccacccggataacgttaagccaatctctgacgttgaaggtactgaaatcaaccaggtattcatcggttcctgcaccaacggtcgtctgtctgatctgcgtgaagctgcgaaatacctgaaaggtcgtgaagttcacaaagacgttaagctgatcgttatcccggcttccaagaaagtattcctgcaggcgctgaaagaaggtatcatcgacatcttcgttaaagcgggtgcgatgatctgtactccgggttgcggtccgtgcctgggtgcacaccagggcgtactggcagaaggtgaaatctgcctgtctactaccaaccgtaacttcaaaggtcgtatgggtcacatcaactcttacatctacctggcttctccgaaaatcgctgctatctctgctgttaaaggttacatcactaacaagctggattaa

SEQ ID NO: 170 - DNA, 513 bp, Escherichia coli
atgatcatcaaaggtcgtgcgcacaagttcggtgacgacgttgacactgacgctatcatcccaggtccgtacctccgtactactgacccgtacgaactggcatctcactgcatggcgggtatcgacgaaaacttcccgaagaaagttaaagaaggtgacgttatcgttgctggcgaaaacttcggttgcggttcttcccgtgagcaggctgttatcgctatcaaatactgcggtatcaaagcggttatcgctaaatctttcgcacgtatcttctaccgtaacgcaatcaacgtaggtctgatcccgatcatcgctaacaccgacgaaatcaaagacggtgacatcgttgaaatcgacctggataaagaagaaatcgttatcactaacaaaaacaaaactatcaagtgcgaaactccgaaaggtctggaacgtgaaatcctggcagctggcggtctggttaactacctgaagaaacgtaagctgattcagtccaagaaaggcgtaaaaacttaa

SEQ ID NO: 171 - DNA, 1221 bp, Saccharomyces cerevisiae
atgaccaaggttttggtcatgttcatggacttcttgtttgaaaactcctggaaggccgtttgtccatacaacccaaagttggacttgaaggacatctacatctacgacaccactttaagagatggtgaacaaaccccaggtgtttgtttcaccaaggaacaaaaattggaaattgccagaaagttggacgaattgggtttgaaacaaatcgaagctggtttcccaatcgtttctgaaagagaagctgacattgtcaagaccattgccaacgaaggtttgaacgctgatatcttagctctatgtagagctttgaagaaggacattgacaaggccatcgaatgtgatgtcgatggtatcatcactttcattgctacttctccattacatttgaaatacaagttcaacaacaaatctttggacgaaatcttggaaatgggtgttgaagctgtcgaatacgccaaggaacacggtttattcgttgctttctctgctgaagatgctaccagaactccaattgaagatttgatcaaggtccacaaggctgctgaagaagctggtgctgaccgtgtccacattgctgacaccactggttgtgccactccacaatccatggaatttatctgtaagactttgaaggaaaacttgaagaaggctcacattggtgttcactgtcacaacgatttcggtttcgctgtcatcaactccatctacggtttgattggtggtgccaaggccgtttccaccaccgtcaacggtatcggtgaaagagctggtaacgctgctttggaagaattgatcatggctttgactgtcttatacgatgtcgatttgggtttgaacttggaagttttgccagaattgtgtagaatggttgaagaatactctggtatcaagatgccaaagaacaagccaattgtcggtgaattggttttcgctcatgaatctggtattcacgttgacgctgtcattgaaaacccattgacctacgaacctttcttgccagaaaagatcggtttgaagagaaacatcctattaggtaagcactctggttgtcgtgctgttgcttacaaattgaaattgatgggtattgactacgacagagaaatgttgtgtgaaattgtcaagaaggtcaaggaaatcagagaagaaggtaagttcatcactgacgaagttttcaaggaaatcgttgaagaagttttgagaaagagaaacaaaaattaa

SEQ ID NO: 172 - DNA, 1263 bp, Saccharomyces cerevisiae
atgactttagtcgaaaagatcttatccaagaaggtcggttacgaagtttgtgccggtgactctattgaagttgaagttgacttggccatgacccacgacggtactaccccattggcttacaaggctttgaaggaaatgtctgactccgtctggaacccagacaagattgttgttgctttcgaccacaacgttccaccaaacaccgtcaaggctgctgaaatgcaaaaattggctttggaatttgtcaagagattcggtatcaagaacttccacaagggtggtgaaggtatctgtcaccaaatcttggctgaaaactacgttttgccaaacatgttcgttgctggtggtgactcccacacttgtacccacggtgctttcggtgcctttgctaccggtttcggtgctactgacatggcttacatctacgctaccggtgaaacctggatcaaggttccaaagactatcagagttgacattgtcggtaagaacgaaaacgtttctgccaaggatatcgtcttgagagtttgtaaggaaattggtagaagaggtgctacttacatggccattgaatacggtggtgaagttgtcaagaacatggacatggacggtagattgactttgtgtaacatggccattgaaatgggtggtaagactggtgtcattgaagctgatgaaatcacctacgactacttgaagaaggaaagaggtctatccgatgaagatatcgccaaattgaagaaggaaagaatcactgttaacagagatgaagctaactactacaaggaaattgaaattgatatcactgacatggaagaacaagttgctgttcctcatcacccagacaatgtcaagccaatttctgacgtcgaaggtactgaaatcaaccaagttttcatcggttcttgtaccaacggtagattatctgatttacgtgaagctgctaagtacttgaaaggtcgtgaagttcacaaggatgtcaaattgattgtcattccagcttccaagaaggttttcttgcaagctttgaaggaaggtatcatcgatatcttcgtcaaggctggtgccatgatctgtaccccaggttgtggtccatgtttgggtgctcatcaaggtgtcttggctgaaggtgaaatctgtttgtccaccaccaacagaaacttcaagggtagaatgggtcacatcaactcttacatctacttggcttctccaaagattgctgccatttctgctgtcaagggttacatcactaacaaattggattaa

SEQ ID NO: 173 - DNA, 513 bp, Saccharomyces cerevisiae
atgatcatcaagggtcgtgctcacaagttcggtgacgatgttgacactgatgctatcattccaggtccatacttgagaaccactgacccatacgaattggcttctcactgtatggctggtattgacgaaaacttcccaaagaaggtcaaggaaggtgatgtcattgttgctggtgaaaactttggttgtggttcttccagagaacaagctgttattgccatcaaatactgtggtatcaaggctgtcattgccaagtctttcgctagaatcttctacagaaacgccatcaacgttggtttgattccaatcattgctaacactgacgaaatcaaggatggtgacattgttgaaatcgatttggacaaggaagaaattgttatcaccaacaagaacaagaccatcaagtgtgaaactccaaagggtttggaaagagaaatcttggctgctggtggtttagtcaactacttgaagaagagaaagttgatccaatccaagaagggtgtcaaaacctaa

SEQ ID NO: 174 - DNA, 1044 bp, Saccharomyces cerevisiae
atgatgaaggtttgtgtcattgaaggtgacggtattggtaaggaagtcattccagaagctatcaagatcttgaatgaattgggtgaatttgaaatcatcaagggtgaagctggtttggaatgtttgaagaaatacggtaacgctttgccagaagataccattgaaaaggccaaggaagctgatatcatcttattcggtgccatcacttctccaaagccaggtgaagttcaaaactacaaatctccaatcatcactttgagaaagatgttccacttgtacgctaacgtcagaccaatcaacaacttcggtattggtcaattgattggtaagattgctgactacgaatttttgaatgccaagaacattgacattgtcatcatcagagaaaacactgaagatttgtacgttggtcgtgaaagattagaaaacgacactgccattgctgaacgtgttatcaccagaaagggttctgaaagaatcatcagattcgctttcgaatacgccatcaagaacaacagaaagaaggtttcctgtatccacaaggctaacgttttgagaatcaccgatggtttattcttggaagttttcaacgaaatcaagaagcactacaacattgaagctgatgactacttggttgactccactgctatgaacttgatcaagcatccagaaaagttcgatgtcattgtcaccaccaacatgttcggtgacatcttatctgacgaagcttctgctttgattggtggtctaggtttggctccatctgccaacattggtgatgacaaggctttattcgaacctgttcacggttctgctccagacattgctggtaagggtattgccaacccaatggcttccatcttgtccattgctatgttgttcgactacatcggtgaaaaggaaaagggtgacttgatcagagaagctgtcaaatactgtttgatcaacaagaaggttactccagatttgggtggtgacttgaaaaccaaggatgtcggtgacgaaatcttgaactacatcagaaagaaattgaaaggctactaa

SEQ ID NO: 175 - DNA, 52 bp, Artificial
  DC-KdcA-F
aaatttggatccgttgaggaggcctcaaaaatgtatactgttggtgattatc

SEQ ID NO: 176 - DNA, 37 bp, Artificial
  DC-KdcA-R
aaatttggcgcgccattacttgttctgctccgcaaac

SEQ ID NO: 177 - DNA, 1161 bp, Artificial
  AksA E. coli codon optimised
atggactggaaagcggtatctccgtacaacccgaaactgaacctgaaagactgctacctgtacgacaccactctgcgtgacggcgagcagactccgggcgtttgcttcactcacgaccagaaactggaaatcgcgaagaaactggacgaactgaaaatcaagcagatcgaagctggcttcccgatcgtttctgaaaacgaacgtaaagcaatcaagtctatcaccggtgaaggtctgaacgctcagatcctggcactctctcgcgtactgaaagaagatatcgacaaagcaatcgaatgcgacgttgacggtatcatcactttcatcgctgcttctccgatgcacctgaaatacaaactgcacaaatctctggatgaagttgaagagatgggtatgaaagcggtagaatacgctaaagaccacggtctgttcgttgcattctctgctgaagatgcaactcgtactccggttgaagatctgatccgtatccacaaaaacgctgaagagcacggtgctaaccgcgttcacatcgctgacactctgggttgcgcaactccgcaggcaatgtaccacatctgctctgaactgtcctccaacctgaagaaagcgcacatcggtgttcactgccacaacgacttcggtttcgctgttatcaactccatctacggtctgatcggtggtgcgaaagcggtatctactaccgttaacggtatcggtgaacgtgctggtaacgctgctatcgaagaaatcgttatggcgctgaaagttctgtacgaccacgacatgggtctgaacactgaaatcctgactgaaatctccaagctggttgaaaactactccaagatccgtatcccggaaaacaagccgctggttggtgaaatggcattctaccacgaatccggtatccacgttgacgctgttctggaaaacccgctgacttacgaaccgttcctgccagaaaaaatcggtcagaagcgtaagatcatcctgggtaagcactctggttgccgtgctgttgctcaccgtctgcaggaactgggtctggaagcatctcgtgaagagctgtgggaaatcgttaagaaaaccaaagaaactcgtgaagaaggtactgaaatctctgacgaagtattcaaaaacatcgttgacaaaatcattaaataa

SEQ ID NO: 178 - DNA, 1020 bp, Artificial
  AksF E. coli codon optimised
atgcgtaacactccgaaaatctgcgttatcaacggtgacggtatcggtaacgaagttatcccggaaaccgttcgcgtactgaacgaaatcggtgacttcgaattcatcgaaactcacgctggttacgaatgcttcaagcgctgcggtgacgctatcccggaaaaaactatcgaaatcgctaaagagtctgactccatcctgttcggttctgtaactactccgaagccgactgaactgaaaaacaagccgtaccgttctccgattctgactctgcgtaaagagctggatctgtacgctaacatccgtccgactttcaacttcaaaaacctggacttcgttatcatccgtgaaaacactgaaggtctgtacgttaagaaagaatactacgacgaaaaaaacgaagttgcaactgctgaacgtatcatctccaaattcggttcttcccgtatcgttaagttcgcattcgactacgcactgcagaacaaccgtaagaaagtttcctgcatccacaaagctaacgttctgcgtatcactgacggtctgttcctgggcgtattcgaagaaatctccaagaaatacgagaagctgggtatcgtttctgacgactacctgatcgacgcaactgcgatgtacctgatccgtaacccgcagatgttcgacgtaatggttaccactaacctgttcggtgacatcctgtctgacgaagctgctggtctgatcggtggtctgggtatgtccccgtctgctaacatcggtgacaaaaacggtctgttcgaaccggttcacggttctgcaccggatatcgctggtaaaggtatctccaacccaatcgcgactatcctgtctgctgcaatgatgctggatcacctgaaaatcaacaaagaagctgaatacatccgtaacgctgttaagaaaaccgttgaatgtaaatacctgactccggacctgggtggtcacctgaaaacttctgaagttactgaaaaaatcatcgaatccatcaaatctcagatgattcagtaa

SEQ ID NO: 179 - DNA, 1257 bp, Artificial
  AksD E. coli codon optimised
atgactctggctgaaaaaatcatctccaaaaacgttggtaaaaacgtttacgctggcgactccgttgaaatcgacgttgacgttgcgatgactcacgacggtactactccgctgaccgttaaagcattcgagcagatctctgacaaagtatgggataacgaaaaaatcgttatcatcttcgaccacaacatcccggctaacacctctaaagctgctaacatgcaagttatcactcgtgaattcatcaagaagcagggtatcaaaaactactacctggacggtgaaggtatctgccaccaggttctgccggaaaaaggtcacgttaagccgaacatgatcatcgctggtgctgactctcacacctgtactcacggtgcattcggtgcattcgcaactggcttcggtgcaactgacatgggttacgtttacgcaactggtaaaacctggctgcgcgtaccagaaaccattcaggttaacgtaactggcgaaaacgaaaacatctccggtaaagacatcatcctgaaaacctgtaaagaagttggtcgtcgcggtgcaacttacctctctctggaatacggtggtaacgcggtacagaacctggatatggacgaacgtatggttctgtctaacatggctatcgaaatgggtggtaaagcgggtatcatcgaagctgacgacaccacttacaaatacctggaaaacgctggcgtttcccgtgaagaaatcctgaacctgaagaaaaacaagatcaaagttaacgaatctgaagaaaactactacaaaactttcgagttcgacatcactgacatggaagagcagatcgcttgcccgcaccacccggacaacgttaaaggcgtttctgaagtttctggtatcgaactggatcaggtattcatcggttcctgcaccaacggtcgtctgaacgatctgcgtatcgctgcgaagcacctgaaaggtaagaaagttaacgaatccactcgtctgatcgttatcccggcttccaagtctatcttcaaagaagcgctgaaagaaggtctgatcgacaccttcgttgactccggtgcgctgatctgtactccgggttgcggtccgtgcctgggtgcacaccagggcgtactgggtgacggtgaagtttgcctggcaactaccaaccgtaacttcaaaggtcgtatgggtaacaccaagtctgaagtttacctctcttctccggcaatcgctgcgaagtctgctgttaaaggttacatcactaacgagtaa

SEQ ID NO: 180 - DNA, 486 bp, Artificial
  AksE E. coli codon optimised
atgaagatcaccggtaaagttcacgtattcggtgacgacatcgacactgacgctatcattccgggtgcttacctgaaaaccactgacgaatacgaactggcttctcactgcatggcgggtatcgacgaagatttcccggaaatggttaaagaaggtgacttcctggttgctggcgaaaacttcggttgcggttcttcccgtgagcaggcaccgatcgctatcaaatactgcggtatcaaagcaatcatcgttgaatccttcgcacgtatcttctaccgtaactgcatcaacctgggcgtattcccgatcgaatgtaaaggtatctccaagcacgttaaagacggtgacctgatcgaactggatctggaaaacaagaaagttatcctgaaagacaaagttctggactgccacatcccgactggtactgcgaaagacatcatggacgaaggtggtctgatcaactacgctaagaagcagaaaaactaa

SEQ ID NO: 181 - DNA, 1161 bp, Methanococcus maripaludis
atggattggaaagctgtatctccgtacaaccctaaattaaatttgaaagactgttatttgtatgatacgacattgagagatggtgaacagactcccggagtttgttttacacatgatcaaaaacttgagatcgccaaaaaactggatgaacttaaaattaaacagatcgaagcgggttttccaattgtttctgaaaacgagagaaaagccatcaaatcaattactggcgaaggattaaatgcacaaattttggcgttatcaagagttttaaaagaggatattgataaagccattgaatgtgatgttgatggaataattacattcattgcagcttcaccaatgcatttgaaatacaaattgcacaaaagcctcgatgaagtcgaagaaatgggtatgaaagccgttgaatacgcaaaagatcacggacttttcgtagcattctctgcagaagatgcgacaagaactcctgttgaagacctcatcagaatccacaaaaatgcagaagaacacggtgccaatagggtgcatattgcagataccctcgggtgtgcaacaccacaggcaatgtatcatatctgctctgaattaagcagtaacttgaaaaaagcacatatcggggtacactgtcacaacgactttgggttcgcagttataaactcgatatacggattaattggtggagcaaaagcggtatctacaacagttaacggaataggcgaaagagcaggaaatgctgcaattgaagaaattgtaatggcattgaaagtactttacgaccacgatatgggattaaatactgaaatactaactgaaatatcgaaactcgttgaaaactattcaaaaattaggattcccgaaaataaacctcttgttggggaaatggcattttaccatgaaagcggaatacatgttgatgcggttttagagaatcctttaacgtatgaaccgtttttacctgaaaaaataggtcaaaaaagaaaaattatacttggaaaacattccggatgcagagcagttgcacacagactgcaagaacttgggcttgaagcttcaagagaagaactttgggaaattgtgaaaaaaactaaagaaaccagagaagaaggtactgaaataagcgacgaagtgtttaaaaacattgtcgataagattataaaataa

SEQ ID NO: 182 - DNA, 1020 bp, Methanococcus maripaludis
atgagaaacactcccaaaatttgtgttattaatggagatggcattggaaacgaagtgattcctgaaacagtgcgcgtcttgaatgaaattggggattttgaatttatagaaacacatgcgggctacgaatgttttaaaagatgtggcgatgcgatacctgaaaagaccatagaaattgcaaaagaatctgattctattctttttggatctgttactaccccaaaaccaactgaattaaaaaataaaccctatagaagtccaatattaactttaagaaaagaactcgacctttatgcaaatataagaccgactttcaacttcaaaaaccttgattttgtgataattcgcgaaaataccgaaggtctttatgtgaaaaaagaatattacgacgaaaaaaatgaagttgcgactgctgaacgaattatttctaaatttggaagctcgagaattgtaaaatttgcttttgattatgcacttcaaaacaatagaaaaaaagtatcctgtattcacaaagcaaatgttttgaggatcacagatgggttattcctaggggtatttgaagaaatatcgaaaaaatatgaaaaattgggaatagtgtctgatgactatttgattgatgcaacagcgatgtatttaattagaaatccgcaaatgtttgatgtcatggttacaacaaatttatttggagatattttatcggatgaagctgctggacttatcggaggacttggaatgtctccttcagcaaatattggtgacaaaaacggattattcgaaccagtgcatggatccgcaccagatattgctggaaaaggaatttcaaacccgattgcaacaattttaagtgctgcaatgatgcttgatcatttaaaaataaataaagaagcggaatacataagaaatgcagttaaaaaaactgttgaatgtaaatacctaactccggatcttgggggacacttaaaaacttctgaagttacagaaaaaatcattgaatcaataaaatctcaaatgattcaatga

SEQ ID NO: 183 - DNA, 1257 bp, Methanococcus maripaludis
atgacacttgctgaaaaaatcatttctaaaaatgttggaaaaaatgtttacgcgggcgatagcgttgaaatagacgtggatgtcgcaatgacgcatgacgggactacccctcttacagtaaaagcttttgagcagatttcagacaaagtttgggataatgaaaagatagttattatttttgaccacaacatccctgcaaacacgtcaaaagctgcgaatatgcaggttataacgagagaatttatcaaaaaacagggaattaaaaattattaccttgatggcgaaggaatatgtcatcaggtacttcctgaaaaaggccacgtgaagccaaacatgataattgcaggagctgacagtcacacctgtactcatggggcattcggtgcttttgcgacaggttttggtgcaactgacatgggttacgtctatgcaaccggaaaaacatggcttagagttcctgaaaccattcaagtaaatgtaaccggagaaaatgaaaatatttctggaaaggacattatcttaaaaacttgtaaggaagttggaagacgtggagcgacatacctgtctttagaatacggcggaaatgcagtccaaaatcttgacatggacgaaagaatggttttatcgaacatggccattgaaatgggcggaaaagctggaattatcgaagctgacgatactacttacaaataccttgaaaatgcaggagtttcaagagaagaaattcttaacttgaaaaaaaataaaataaaagttaatgaatccgaagaaaattactacaaaacatttgaatttgatataaccgatatggaagaacagattgcttgcccgcaccaccctgacaatgtaaaaggagtttctgaagtatcaggaattgaattagatcaggtattcatcggatcttgtacaaacggaagattaaacgatttaagaattgctgcaaaacatttgaaaggaaaaaaagttaatgaaagcacccgactaattgtaattcctgcatcaaaatcaatctttaaagaagcgttaaaagaaggattaatcgatacttttgtagattctggagcattaatctgcactcctggatgcggaccatgccttggagcccatcagggtgttttaggtgatggggaagtatgtcttgctacaaccaataggaactttaaaggaagaatgggaaacacaaaatcggaagtttacctctcatctcctgcaatagctgcaaaatccgcagttaaaggatacattaccaatgaataa

SEQ ID NO: 184 - DNA, 486 bp, Methanococcus maripaludis
atgaaaataacaggcaaggtgcacgtatttggggatgacatcgacacagatgcgataattcctggcgcttatttaaaaacaactgatgaatatgagcttgcatcacactgtatggctggaatcgatgaagattttccagaaatggtcaaagaaggcgactttttggtagcaggtgagaatttcggatgcggaagttcgagagagcaagctccaattgcaataaaatactgcggaatcaaggcaataattgttgaaagttttgcaaggatattttatagaaattgtattaatcttggagtttttccaattgaatgcaaaggaatatcaaaacacgtgaaagatggagatttaatagaattggatctcgaaaataaaaaagtaattttaaaggacaaggttctagactgccacattccaaccggaactgcaaaagacataatggatgaaggcgggcttataaattacgcaaagaaacagaaaaactaa

SEQ ID NO: 185 - DNA, 1113 bp, Homo sapiens
atgctcccccggctaatttgtatcaatgattatgaacaacatgctaaatcagtacttccaaagtctatatatgactattacaggtctggggcaaatgatgaagaaactttggctgataatattgcagcattttccagatggaagctgtatccaaggatgctccggaatgttgctgaaacagatctgtcgacttctgttttaggacagagggtcagcatgccaatatgtgtgggggctacggccatgcagcgcatggctcatgtggacggcgagcttgccactgtgagagcctgtcagtccctgggaacgggcatgatgttgagttcctgggccacctcctcaattgaagaagtggcggaagctggtcctgaggcacttcgttggctgcaactgtatatctacaaggaccgagaagtcaccaagaagctagtgcggcaggcagagaagatgggctacaaggccatatttgtgacagtggacacaccttacctgggcaaccgtctggatgatgtgcgtaacagattcaaactgccgccacaactcaggatgaaaaattttgaaaccagtactttatcattttctcctgaggaaaattttggagacgacagtggacttgctgcatatgtggctaaagcaatagacccatctatcagctgggaagatatcaaatggctgagaagactgacatcattgccaattgttgcaaagggcattttgagaggtgatgatgccagggaggctgttaaacatggcttgaatgggatcttggtgtcgaatcatggggctcgacaactcgatggggtgccagccactattgatgttctgccagaaattgtggaggctgtggaagggaaggtggaagtcttcctggacgggggtgtgcggaaaggcactgatgttctgaaagctctggctcttggcgccaaggctgtgtttgtggggagaccaatcgtttggggcttagctttccagggggagaaaggtgttcaagatgtcctcgagatactaaaggaagaattccggttggccatggctctgagtgggtgccagaatgtgaaagtcatcgacaagacattggtgaggaaaaatcctttggccgtttccaagatctga

SEQ ID NO: 186 - protein, 370 aa, Homo sapiens
MLPRLICINDYEQHAKSVLPKSIYDYYRSGANDEETLADNIAAFSRWKLYPRMLRNVAETDLSTSVLGQRVSMPICVGATAMQRMAHVDGELATVRACQSLGTGMMLSSWATSSIEEVAEAGPEALRWLQLYIYKDREVTKKLVRQAEKMGYKAIFVTVDTPYLGNRLDDVRNRFKLPPQLRMKNFETSTLSFSPEENFGDDSGLAAYVAKAIDPSISWEDIKWLRRLTSLPIVAKGILRGDDAREAVKHGLNGILVSNHGARQLDGVPATIDVLPEIVEAVEGKVEVFLDGGVRKGTDVLKALALGAKAVFVGRPIVWGLAFQGEKGVQDVLEILKEEFRLAMALSGCQNVKVIDKTLVRKNPLAVSKI

SEQ ID NO: 187 - DNA, 1113 bp, Artificial
  human sequence codon optimised
atgctgccacgtctgatttgtattaacgattacgaacaacacgcgaagagcgtactgccgaaatccatttacgattattaccgttctggtgcaaacgatgaagaaacgctggctgataacatcgccgctttttcccgttggaaactgtacccacgtatgctgcgtaacgttgccgaaaccgacctgtccaccagcgtcctgggtcagcgtgtgtccatgccaatctgcgtgggtgcaaccgcaatgcagcgtatggcacacgttgacggcgaactggcaaccgtccgtgcgtgccagagcctgggtaccggtatgatgctgagcagctgggctacctctagcatcgaggaagtggcagaagctggtccggaagcactgcgctggctgcagctgtacatctacaaagatcgcgaagtcactaagaaactggtgcgccaggcggaaaagatgggttacaaggcaatctttgtgactgttgacaccccgtacctgggtaaccgcctggatgacgttcgtaaccgcttcaagctgccgccgcagctgcgtatgaagaactttgaaaccagcaccctgtccttttccccagaagaaaatttcggtgatgactctggtctggccgcgtacgtcgcgaaagctatcgatccgtccatctcctgggaagatatcaaatggctgcgtcgtctgacttccctgccgatcgttgctaagggtattctgcgtggtgacgacgcgcgtgaagctgttaaacatggtctgaacggcattctggtaagcaaccatggcgcacgccagctggatggtgtacctgctactattgatgtgctgccggaaatcgtggaagcggttgaaggtaaagttgaagtgttcctggacggtggtgtgcgcaaaggcaccgatgtactgaaagcactggcgctgggtgcgaaagccgtctttgttggccgtcctattgtttggggtctggcattccagggtgagaaaggtgtacaggacgttctggagatcctgaaagaggagttccgcctggctatggcgctgtctggttgtcaaaacgtgaaagtaatcgataaaaccctggtacgtaaaaaccctctggcagtaagcaagatctaa

SEQ ID NO: 188 - DNA, 1125 bp, Aerococcus viridans
atgaataacaatgacattgaatataatgcacctagtgaaatcaagtacattgatgttgtcaatacttacgacttagaagaagaagcaagtaaagtggtaccacatggtggttttaactatattgccggtgcatctggtgatgagtggactaaacgcgctaatgaccgtgcttggaaacataaattactatacccacgtctagcgcaagatgttgaagcgcccgatacaagtactgaaattttaggtcataaaattaaagccccattcatcatggcaccaattgctgcacatggtttagcccacactactaaagaagctggtactgcacgtgcagtttcagaatttggtacaattatgtccatctcagcttattctggtgcaacatttgaagaaatttctgaaggcttaaatggcggaccccgttggttccaaatctatatggctaaagatgaccaacaaaaccgtgatatcttagacgaagctaaatctgatggtgcaactgctatcatccttacagctgactcaactgtttctggaaaccgtgaccgtgatgtgaagaataaattcgtttacccatttggtatgccaattgttcaacgttacttacgtggtacagcagaaggtatgtcattaaacaatatctacggtgcttcaaaacaaaaaatctcaccaagagatattgaggaaatcgccggtcattctggattaccagtattcgttaaaggtattcaacacccagaagatgcagatatggcaatcaaacgtggtgcatcaggtatctgggtatctaaccacggtgctcgtcaactatatgaagctccaggttcatttgacacccttccagctattgctgaacgtgtaaacaaacgtgtaccaatcgtctttgattcaggtgtacgtcgtggtgaacacgttgccaaagcgctagcttcaggggcagacgttgttgctttaggacgcccagtcttatttggtttagctttaggtggctggcaaggtgcttactcagtacttgactacttccaaaaagacttaacacgcgtaatgcaattaacaggttcacaaaatgtggaagacttgaagggtctagatttattcgataacccatacggttatgaatactag

SEQ ID NO: 189 - protein, 374 aa, Aerococcus viridans
MNNNDIEYNAPSEIKYIDVVNTYDLEEEASKVVPHGGFNYIAGASGDEWTKRANDRAWKHKLLYPRLAQDVEAPDTSTEILGHKIKAPFIMAPIAAHGLAHTTKEAGTARAVSEFGTIMSISAYSGATFEEISEGLNGGPRWFQIYMAKDDQQNRDILDEAKSDGATAIILTADSTVSGNRDRDVKNKFVYPFGMPIVQRYLRGTAEGMSLNNIYGASKQKISPRDIEEIAGHSGLPVFVKGIQHPEDADMAIKRGASGIWVSNHGARQLYEAPGSFDTLPAIAERVNKRVPIVFDSGVRRGEHVAKALASGADVVALGRPVLFGLALGGWQGAYSVLDYFQKDLTRVMQLTGSQNVEDLKGLDLFDNPYGYEY

SEQ ID NO: 190 - DNA, 1125 bp, Artificial
  LAOX-8C A. viridans codon optimised
atgaacaacaacgacatcgaatataacgctccttctgaaatcaaatatatcgacgtggttaacacctatgacctggaggaagaagcgtctaaggtcgtaccgcacggtggtttcaattacattgcaggtgcctctggtgatgaatggaccaaacgcgcaaacgatcgtgcatggaaacacaaactgctgtatccgcgcctggcccaggatgtggaagcaccggatacttccactgaaatcctgggtcacaaaatcaaggcaccgtttattatggctccgatcgcagcgcacggcctggcacacaccaccaaagaagctggcaccgctcgtgcggtttctgagttcggcaccattatgtctatctctgcgtatagcggtgccactttcgaggaaatttccgagggcctgaacggtggcccgcgttggtttcagatttacatggcgaaagatgaccagcagaaccgcgatatcctggatgaagccaaatctgacggcgcgactgctatcatcctgaccgcggactctaccgtatccggtaaccgtgaccgtgatgtgaagaacaagttcgtctatcctttcggtatgccgattgttcagcgctatctgcgcggtaccgctgagggtatgagcctgaacaacatctatggtgcgtccaaacagaaaatcagcccacgtgacatcgaagaaattgctggtcatagcggtctgccggtgtttgtgaaaggtatccagcatccagaagatgcggacatggcaatcaaacgtggtgcgtctggcatctgggttagcaaccacggtgcgcgtcagctgtacgaagctccgggtagcttcgataccctgccggccatcgcggaacgtgtgaataaacgcgtgccgatcgttttcgattccggtgtgcgtcgtggtgaacatgtggcaaaagcactggcgtctggcgctgatgtcgtagcactgggccgtccagtgctgttcggtctggctctgggtggctggcagggcgcttactccgtcctggattactttcagaaagacctgacccgtgttatgcagctgaccggttcccagaacgtagaggacctgaaaggcctggacctgttcgacaacccttacggttacgaatactaa

SEQ ID NO: 191 - protein, 314 aa, Corynebacterium glutamicum
MKETVGNKIVLIGAGDVGVAYAYALINQGMADHLAIIDIDEKKLEGNVMDLNHGVVWADSRTRVTKGTYADCEDAAMVVICAGAAQKPGETRLQLVDKNVKIMKSIVGDVMDSGFDGIFLVASNPVDILTYAVWKFSGLEWNRVIGSGTVLDSARFRYMLGELYEVAPSSVHAYIIGEHGDTELPVLSSATIAGVSLSRMLDKDPELEGRLEKIFEDTRDAAYHIIDAKGSTSYGIGMGLARITRAILQNQDVAVPVSALLHGEYGEEDIYIGTPAVVNRRGIRRVVELEITDHEMERFKHSANTLREIQKQFF

SEQ ID NO: 192 - protein, 329 aa, Escherichia coli
MKLAVYSTKQYDKKYLQQVNESFGFELEFFDFLLTEKTAKTANGCEAVCIFVNDDGSRPVLEELKKHGVKYIALRCAGFNNVDLDAAKELGLKVVRVPAYDPEAVAEHAIGMMMTLNRRIHRAYQRTRDANFSLEGLTGFTMYGKTAGVIGTGKIGVAMLRILKGFGMRLLAFDPYPSAAALELGVEYVDLPTLFSESDVISLHCPLTPENYHLLNEAAFEQMKNGVMIVNTSRGALIDSQAAIEALKNQKIGSLGMDVYENERDLFFEDKSNDVIQDDVFRRLSACHNVLFTGHQAFLTAEALTSISQTTLQNLSNLEKGETCPNELV

SEQ ID NO: 193 - protein, 312 aa, Escherichia coli
MKVAVLGAAGGIGQALALLLKTQLPSGSELSLYDIAPVTPGVAVDLSHIPTAVKIKGFSGEDATPALEGADVVLISAGVARKPGMDRSDLFNVNAGIVKNLVQQVAKTCPKACIGIITNPVNTTVAIAAEVLKKAGVYDKNKLFGVTTLDIIRSNTFVAELKGKQPGEVEVPVIGGHSGVTILPLLSQVPGVSFTEQEVADLTKRIQNAGTEVVEAKAGGGSATLSMGQAAARFGLSLVRALQGEQGVVECAYVEGDGQYARFFSQPLLLGKNGVEERKSIGTLSAFEQNALEGMLDTLKKDIALGEEFVNK

SEQ ID NO: 194 - protein, 312 aa, Bacillus subtilis
MGNTRKKVSVIGAGFTGATTAFLIAQKELADVVLVDIPQLENPTKGKALDMLEASPVQGFDAKITGTSNYEDTAGSDIVVITAGIARKPGMSRDDLVSTNEKIMRSVTQEIVKYSPDSIIVVLTNPVDAMTYAVYKESGFPKERVIGQSGVLDTARFRTFVAEELNLSVKDVTGFVLGGHGDDMVPLVRYSYAGGIPLETLIPKERIDAIVERTRKGGGEIVNLLGNGSAYYAPAASLTEMVEAILKDQRRVLPTIAYLEGEYGYEGIYLGVPTIVGGNGLEQIIELELTDYERAQLNKSVESVKNVMKVLS

SEQ ID NO: 195 - protein, 365 aa, Pichia stipitis
MTLKQQVLFVGKPNTNTEAYKKFSANFEVINYKITSKSQLIEDFEGRLRYIEAIYAGWGGFDGVGGFQGEVLRHCPPNVKVVAICSIGHDGYDTEGMSKRGITLTNVPSVIASEAVADLVLYNTLSSFRNFKMFEKNLGGKLTNTGALRTALVRGEFDQFNGVPVIKPTVGGAFASSCCGRDILSPRGHNVVIVGFGSIGKLIGERLACIGMNIHYVKRSKLSEQEEASLGYKVTYHATLKDTKNIADLVVIACPGTAHTRHMVNEEMINDFAKPFRLINIGRGYVVDEKALVNGLQSGKILFAGLDVFENEPSINPDLLNRQDVVLTPHIGSSTTENFNYTAAAAMFNIETVLYDREDTITRVN

SEQ ID NO: 196 - protein, 424 aa, Pseudomonas putida
MSVDPQKLLRELFDTAIAAAHPRQVLEPYLPADRSGRVIVIGAGKAAAAMAEVVEKSWQGEVSGLVVTRYGHGANCQKIEVVEAAHPVPDAAGLAVAKRVLELVSNLNEEDRVIFLLSGGGSALLALPAEGLTLADKQQINKALLKSGATIGEMNCVRKHLSAIKGGRLAKACWPATVYTYAISDVPGDLATVIASGPTVADPSTSADALAILKRYNIEAPKAVIDWLNNPASETVKADDPALARSHFQLIAKPQQSLEAAAVKARQAGFSPLILGDLEGESREVAKVHAGIARQIVQHGQPLKAPCVILSGGETTVTVRGNGRGGRNAEFLLSLTESLKGLPGVYALAGDTDGIDGSEENAGAFMTPASYASAEALGLSASDELDNNNGYGYFAALDALIVTEPTRTNVNDFRAILILETAQS

SEQ ID NO: 197 - protein, 347 aa, Corynebacterium glutamicum
MPEVTVNAQQLTVLCTDILTKTGVPAADAHLVGDSLVQADLWGHPSHGVLRLPWYVRRLHSGAMTTHAHVEVLNDLGAVLALDGHNGIGQVLADHARKEAVTRAMMFGIGAVSVRNSNHFGTAMYYTRKAAAQGCVSILTTNASPAMAPWGGREKRIGTNPWSIAAPFGETATVVDIANTAVARGKIYHARQTNMPIPETWAITSEGAPTTDPAEAINGVVLPMAGHKGYAISFMMDVLSGVLTGSQHSTKVHGPYDPTPPGGAGHLFIALDVAAFRDPQDFDDALSDLVGEVKSTPKAQNTEEIFYPGESEDRAHRKNSAHGISLPEKTWMELQELAIENHVVTHR

SEQ ID NO: 198 - protein, 315 aa, Vibrio fischeri
MKVSYYEVKERLIRKFIASGLAWDDANWVTDVLISSEQRGDKSHGIKHAKNIFDVINSECYIAQAPIIHDERSITILDGQNSIGPIVAKQAIDIAIKKAKKYGTAAISLRSSNHLFSLSHYVRYIANNNMIGFICSSSSPAMAAPNSLNATIGTNPFAFGAPSSKDPIVIDMSSTNVARGKIKEYKDAELDIPVSWALDEYGNPTTCAIEALKGTLSPLGGYKGFALGCMIDIFSSVLSGSAFSTQITGTSLHMEEADVNKKGDFLFVLDISKFIQLSEFKIRMDEFIHIIESNGGYIPGTNYINNQFADIEILN

SEQ ID NO: 199 - protein, 354 aa, Bacillus weihenstephanensis
MEKRIVCLAGDGVGPEIMESAKEVLHMVERLYGHHFHLQDEYFGGAAIDLNGQPLPQRTLAACLASDAVLLGAVGGPRWDDAKERPEKGLLALRKGLGVFANVRPVTVESATAHLSPLKNADEIDFVVVRELTGGIYFSYPKERTEESATDTLTYHRHEIERIVSYAFQLASKREKKVTSIDKANVLESSKLWRAVTEEVALRYPNVELEHILVDAAAMELIRNPRRFDVIVTENLFGDILSDEASVLAGSLGMLPSASHAENGPSLYEPIHGSAPDIAGKNKANPIAMMRSVAMMLGQSFGLTREGYAIEEAISAVLQSGKCTADIGGNETTTSFTRAVIQEMEEQALVGRGR

SEQ ID NO: 200 - protein, 349 aa, Zymomonas mobilis
MRIALLAGDGIGPEITAEAVKILKAVVGQEIEFDEALIGGAAWKVTGSPLPEETLKLCKNSDAILFGSVGDPECDHLERALRPEQAILGLRKELDLFANLRPARLFPELQAESPLKENIVTGTDLMIVRELTGDVYFGTPRGQRKDDQNRREGFDTMRYNEDEVKRIARIGFETARSRSGNLCSIDKSNVLETSQLWRTVVLEIAQEYPDVELSHMYVDNAAMQLVRAPDQFDVIVTGNLFGDILSDLASACVGSIGLLPSASLNSEGKGLYEPIHGSAPDIAGLGKANPLATILSGAMMLRYSLKREADADRIEKAVSTALEKGARTADLGGKMTTSEMGNAVLAALN

SEQ ID NO: 201 - protein, 361 aa, Escherichia coli
MMKTMRIAAIPGDGIGKEVLPEGIRVLQAAAERWGFALSFEQMEWASCEYYSHHGKMMPDDWHEQLSRFDAIYFGAVGWPDTVPDHISLWGSLLKFRREFDQYVNLRPVRLFPGVPCPLAGKQPGDIDFYVVRENTEGEYSSLGGRVNEGTEHEVVIQESVFTRRGVDRILRYAFELAQSRPRKTLTSATKSNGLAISMPYWDERVEAMAENYPEIRWDKQHIDILCARFVMQPERFDVVVASNLFGDILSDLGPACTGTIGIAPSANLNPERTFPSLFEPVHGSAPDIYGKNIANPIATIWAGAMMLDFLGNGDERFQQAHNGILAAIEEVIAHGPKTPDMKGNATTPQVADAICKIILR

SEQ ID NO: 202 - protein, 362 aa, Aspergillus niger
MTTETTTYRIASIPGDGIGEEVVRATIEVINKLAQTLNTFNIEFTHLPWGTEYYKQHGRYVSEGYLDTLRQFDAGLFGSVGHPDVPDHVSLWGLLLALRSPLQLYANVRPVRTFPGTKSPLTTAVNGIDWVLVRENSEGEYCGQGGRSHTGQPWEAATEVAIFTRVGVERIMRFAFETARSRPRRHLTVVTKSNAMRHGMVLWDEVAEEVAKDFPDVTWDKMLVDAMTLRMISKPESLDTIVGTNLHMDILSDLAAGLAGSIGVAPSSNLDPTRKNPSLFEPVHGSAFDIMGKGVANPVATFWSAAEMLAWLGEKDAAKKLMDCVEKVCAAGILTPDLGGSANTQGVVDAVCKEIEQQLASS

SEQ ID NO: 203 - protein, 591 aa, Saccharomyces cerevisiae
MLKYKPLLKISKNCEAAILRASKTRLNTIRAYGSTVPKSKSFEQDSRKRTQSWTALRVGAILAATSSVAYLNWHNGQIDNEPKLDMNKQKISPAEVAKHNKPDDCWVVINGYVYDLTRFLPNHPGGQDVIKFNAGKDVTAIFEPLHAPNVIDKYIAPEKKLGPLQGSMPPELVCPPYAPGETKEDIARKEQLKSLLPPLDNIINLYDFEYLASQTLTKQAWAYYSSGANDEVTHRENHNAYHRIFFKPKILVDVRKVDISTDMLGSHVDVPFYVSATALCKLGNPLEGEKDVARGCGQGVTKVPQMISTLASCSPEEIIEAAPSDKQIQWYQLYVNSDRKITDDLVKNVEKLGVKALFVTVDAPSLGQREKDMKLKFSNTKAGPKAMKKTNVEESQGASRALSKFIDPSLTWKDIEELKKKTKLPIVIKGVQRTEDVIKAAEIGVSGVVLSNHGGRQLDFSRAPIEVLAETMPILEQRNLKDKLEVFVDGGVRRGTDVLKALCLGAKGVGLGRPFLYANSCYGRNGVEKAIEILRDEIEMSMRLLGVTSIAELKPDLLDLSTLKARTVGVPNDVLYNEVYEGPTLTEFEDA

SEQ ID NO: 204 - protein, 396 aa, Escherichia coli [sequence omitted]

SEQ ID NO: 205 - protein, 587 aa, Saccharomyces cerevisiae
MLWKRTCTRLIKPIAQPRGRLVRRSCYRYASTGTGSTDSSSQWLKYSVIASSATLFGYLFAKNLYSRETKEDLIEKLEMVKKIDPVNSTLKLSSLDSPDYLHDPVKIDKVVEDLKQVLGNKPENYSDAKSDLDAHSDTYFNTHHPSPEQRPRIILFPHTTEEVSKILKICHDNNMPVVPFSGGTSLEGHFLPTRIGDTITVDLSKFMNNVVKFDKLDLDITVQAGLPWEDLNDYLSDHGLMFGCDPGPGAQIGGCIANSCSGTNAYRYGTMKENIINMTIVLPDGTIVKTKKRPRKSSAGYNLNGLFVGSEGTLGIVTEATVKCHVKPKAETVAVVSFDTIKDAAACASNLTQSGIHLNAMELLDENMMKLINASESTDRCDWVEKPTMFFKIGGRSPNIVNALVDEVKAVAQLNHCNSFQFAKDDDEKLELWEARKVALWSVLDADKSKDKSAKIWTTDVAVPVSQFDKVIHETKKDMQASKLINAIVGHAGDGNFHAFIVYRTPEEHETCSQLVDRMVKRALNAEGTCTGEHGVGIGKREYLLEELGEAPVDLMRKIKLAIDPKRIMNPDKIFKTDPNEPANDYR

SEQ ID NO: 206 - protein, 477 aa, Gluconobacter oxydans
MPEPVMTASSASAPDRLQAVLKALQPVMGERISTAPSVREEHSHGEAMNASNLPEAVVFAESTQDVATVLRHCHEWRVPVVAFGAGTSVEGHVVPPEQAISLDLSRMTGIVDLNAEDLDCRVQAGITRQTLNVEIRDTGLFFPVDPGGEATIGGMCATRASGTAAVRYGTMKENVLGLTVVLATGEIIRTGGRVRKSSTGYDLTSLFVGSEGTLGIITEVQLRLHGRPDSVSAAICQFESLHDAIQTAMEIIQCGIPITRVELMDSVQMAASIQYSGLNEYQPLTTLFFEFTGSPAAVREQVETTEAIASGNNGLGFAWAESPEDRTRLWKARHDAYWAAKAIVPDARVISTDCIVPISRLGELIEGVHRDIEASGLRAPLLGHVGDGNFHTLIITDDTPEGHQQALDLDRKIVARALSLNGSCSGEHGVGMGKLEFLETEHGPGSLSVMRALKNTMDPHHILNPGKLLPPGAVYTG

SEQ ID NO: 207 - protein, 433 aa, Caenorhabditis elegans
MLNRGTFQVFRGISGPPKKSVDLPKYDLVIVGGGIVGCATARQLLIEKPQLKVALIEKEKELAVHQSGHNSGVIHAGIYYTPGSLKAKLCVEGLDLSYEFFDKEKVPYKKTGKLIVAVEPEEVPRLDALFSRAQTNGCRDIEMIDSSKITELEPHCRGLKALWSPHTGIVDWGYVTKRFGEDFEKRGGKIYTSYPLEKISDNHDPGYPIRVSSGPALAEFETKNLITCAGLQSDRVAALSGCSTDPKIVPFRGEYLLLKPEKRHLVKTNIYPVPDPRFPFLGVHFTPRMNGDIWLGPNAVLAYKREGYSYFSISPSDLLESLSYSGMQKLVKKHFTFGIKELYRGVWIAAQVKQLQRFIPELKLSDVTRGPAGVRAQAMDSAGNLVDDFVFDSGTGKLSPLLMHVRNAPSPAATSSLAIAKMITSEAINRFKL

SEQ ID NO: 208 - protein, 455 aa, Drosophila melanogaster
MAQVRLLVQGLRRSLLNVGVAAPNESTATHKRSQHSSSSCGDYDLVVVGGGIVGAASAREIVLRHPSLKVAVLEKECKLAKHQSGHNSGVIHAGIYYKPGTLKARLCVEGMHLAYAYLDEKKIPYKKTGKLIVATDEKEVKLLKDLEKRGIANNVPDLRMIEGSEIQEIEPYCQGVMALHSPHTGIVDWGLVTEHYGQDFKQCGGDIYLDFNVSKFTETKEGTDYPVTIHGAKPGQTVRTKNVLTCGGLQSDLLAEKTGCPRDPRIVPFRGEYLLLTKEKQHMVKGNIYPVPDPRFPFLGVHFTPRMDGSIWLGPNAVLALKREGYTWGDINLFELFDALRYPGFVKMASKYIGFGLSEMSKSWFINLQIKALQKYIPDITEYDIQRGPAGVRAQAMDLDGNLVDDFVFDRGQGSGALAKRVLHCRNAPSPGATSSLAIAKMIADKIENEFSIGK

SEQ ID NO: 209 - protein, 321 aa, Bacillus subtilis
MMNKHVNKVALIGAGFVGSSYAFALINQGITDELVVIDVNKEKAMGDVMDLPHGKAFGLQPVKTSYGTYEDCKDADIVCICAGANQKPGETRLELVEKNLKIFKGIVSEVMASGFDGIFLVATNPVDILTYATWKFSGLPKERVIGSGTTLDSARFRFMLSEYFGAAPQNVHAHIIGEHGDTELPVWSHANVGGVPVSELVEKNDAYKQEELDQIVDDVKNAAYHIIEKKGATYYGVAMSLARITKAILHNENSILTVSTYLDGQYGADDVYIGVPAVVNRGGIAGITELNLNEKEKEQFLHSAGVLKNILKPHFAEQKVN

SEQ ID NO: 210 - protein, 342 aa, Pseudomonas putida
MTHPRHALQRSSTMRALLFSSQHYDQESFTKAAGGTALELHFQPARLTLDTAALADGFEVVCAFINDELDAPVLQRLAAAGTRLIALRSAGYNHVDLAAAQRLGLAVVRVPAYSPHAVAEHAVALILALNRRLHRAYNRTREGDFTLHGLTGFDLHGKTVGVVGTGQIGVAFARIMAGFGCQLLAYDPYPNPELLALGARYLPLPELLREARIISLHCPLTEHTRHLINAQSLAQLQPGAMLINTGRGALVDTPALIDALKSGQLGYLGLDVYEEEAQLFFEDRSDLPLQDDVLARLLTFPNVIITAHQAFLTREALDAIAATTLDNINRWAAGNPQNLVMG